(19) United States
(12) Patent Application Publication
(10) Pub. No.: US 2024/0067662 A1
(43) Pub. Date: Feb. 29, 2024

(54) KRAS INHIBITORS

(71) Applicant: Bristol-Myers Squibb Company, Princeton, NJ (US)

(72) Inventors: Matthew Leo CONDAKES, Brookline, MA (US); Rita Lee CIVIELLO, Killingworth, CT (US); Joanne Jewett BRONSON, Wenham, MA (US)

(21) Appl. No.: 18/331,831

(22) Filed: Jun. 8, 2023

Related U.S. Application Data
(60) Provisional application No. 63/482,726, filed on Feb. 1, 2023.
(60) Provisional application No. 63/351,132, filed on Jun. 10, 2022.

(51) Int. Cl. C07D 519/00; C07D 471/04

(52) U.S. Cl. CPC C07D 519/00; C07D 471/04

(57) ABSTRACT

The present disclosure provides KRAS inhibitors. Methods of treating cancers using the compounds are also provided.

### CROSS-REFERENCE TO RELATED APPLICATIONS

This application claims the priority benefit of U.S. Provisional Application No. 63/482,726, filed Feb. 1, 2023, and U.S. Provisional Application No. 63/351,132, filed Jun. 10, 2022, which are each incorporated by reference herein in their entireties.

### FIELD

The present disclosure provides KRAS inhibitors. Methods of treating cancers using the inhibitors are also provided.

### BACKGROUND

The KRAS oncogene is a member of the RAS family of GTPases that are involved in numerous cellular signaling processes. KRAS mutations are gain-of-function mutations that are present in up to 30% of all tumors, including as many as 90% of pancreatic cancers. KRAS serves as a molecular switch cycling between inactive (GDP-bound) and active (GTP-bound) states to transduce upstream cellular signals received from multiple tyrosine kinases to downstream effectors to regulate a wide variety of processes, including cellular proliferation. Single nucleotide substitutions that result in missense mutations at codons 12 and 13 of the KRAS primary amino acid sequence comprise approximately 40% of KRAS driver mutations in lung adenocarcinoma, with a G12C transversion being the most common activating mutation. KRAS G12C mutations occur in about 13% of lung adenocarcinomas and about 3% of colorectal adenocarcinomas and are also present in cancers of the breast, bladder, cervix, ovaries, pancreas and uterus.

Despite several unsuccessful efforts to target KRAS, compounds that inhibit KRAS activity, including those that disrupt effectors such as guanine nucleotide exchange factors and target KRAS G12C, are highly desirable. Clearly there remains a continued interest and effort to develop inhibitors of KRAS, particularly inhibitors of activating KRAS mutants, such as KRAS G12C.

### SUMMARY

The present disclosure is based, in part, on the discovery that unlike other KRAS G12C inhibitors, compounds of the disclosure target the active, KRAS G12C^{(ON)} form of KRAS G12C protein. By inhibiting the G12C^{ON} form of KRAS, G12C, it is expected that the claimed compounds will decrease a cancer's resistance to KRAS G12C inhibition and/or demonstrate increased potency in the clinic. Without being bound by a theory, the inhibition of G12C^{ON} form of KRAS G12C may be a result of the substituent at position 4 of the pyridopyrimidine ring in formula (I).

In a first aspect, the present disclosure provides a compound of formula (I):

or a pharmaceutically acceptable salt thereof, wherein:

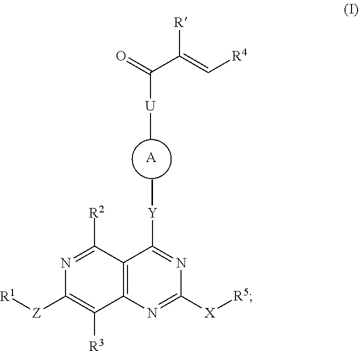

U is a bond or NH;
Z is a bond, O, NR^{e} or CR^{e}R^{f}, wherein R^{e} and R^{f} are independently hydrogen or C₁-C₃alkyl;
R¹ is aryl or heteroaryl, wherein the aryl and the heteroaryl are optionally substituted with one, two, three, four, or five substituents independently selected from C₁-C₃alkoxy, C₁-C₃alkyl, C₂-C₄alkenyl, C₂-C₄alkynyl, amino, amino C₁-C₃alkyl, cyano, C₃-C₄cycloalkyl, halo, halo C₁-C₃alkyl, hydroxy, and hydroxy C₁-C₃alkyl;
R² and R³ are independently selected from hydrogen, C₁-C₃alkoxy, C₁-C₃alkyl, cyano, halo, halo C₁-C₃alkyl, —C(O)NH₂, —C(O)NH(C₁-C₃alkyl), —C(O)N(C₁-C₃alkyl)₂, and hydroxy;
Y is a bond, O, NR^{g}(CR^{e}R^{f})m, NR^{f}, or CR^{e}R^{f}, wherein m is 1, 2, or 3, and wherein R^{e}, R, and R^{g} are independently hydrogen or C₁-C₃alkyl;
A is a four- to ten-membered nitrogen-containing monocyclic or bicyclic bridged, fused, or spirocyclic saturated, unsaturated, or partially unsaturated ring system optionally containing one or two heteroatoms independently selected from nitrogen, oxygen, and sulfur, wherein the ring system is optionally substituted with one, two, or three groups independently selected from C₁-C₃alkoxy, C₁-C₃alkoxyalkyl, C₁-C₃alkyl, cyano, cyano C₁-C₃alkyl, halo, halo C₁-C₃alkyl, amino, amino C₁-C₃alkyl, hydroxy, hydroxy C₁-C₃alkyl, and oxo;
R′ is halo;
R⁴ is an aryl or heteroaryl ring; wherein the ring is optionally substituted with one, two, or three substituents independently selected from C₂-C₄alkenyl, C₁-C₃alkoxy, C₁-C₃alkoxy C₁-C₆alkyl, C₁-C₃alkyl, cyano, cyano C₁-C₃alkyl, halo, halo C₁-C₃alkoxy, halo C₁-C₃alkyl, nitro, and oxo;
X is O or NR¹⁶, wherein R¹⁶ is hydrogen or C₁-C₃alkyl;
R⁵ is selected from hydrogen, C₁-C₆alkoxy C₁-C₆alkyl, C₁-C₆alkyl, aryl, aryl C₁-C₆alkyl, carboxy C₁-C₆alkyl, C₃-C₆cycloalkyl, C₃-C₆cycloalkyl C₁-C₆alkyl, di(C₁-C₃alkyl)amino C₂-C₆alkyl, halo C₁-C₆alkyl, heteroaryl, heteroaryl C₁-C₆alkyl, heterocyclyl, heterocyclyl C₁-C₆alkyl, hydroxy C₁-C₆alkyl, NR^{a}R^{b}—C(O)—C₁-C₆alkyl), NR^{a}R^{b} C₁-C₆alkyl, wherein the aryl, the aryl part of the aryl C₁-C₆alkyl, the C₃-C₆cycloalkyl, the cycloalkyl part of the C₃-C₆cycloalkyl C₁-C₆alkyl, the heteroaryl, the heteroaryl part of the heteroaryl C₁-C₆alkyl, the heterocyclyl, the heterocyclyl part of the heterocyclyl C₁-C₆alkyl, are optionally substituted with one, two, three, or four groups independently selected from C₁-C₃alkoxy, C₁-C₃alkyl, (C₁-C₆alkyl)amino, (C₁-C₆alkyl)amino C₁-C₃alkyl, amino, amino C₁-C₃alkyl, azido C₁-C₆alkyl, carboxy, cyano, di(C₁-C₆alkyl)amino, di(C₁-C₆alkyl)amino C₁-C₃alkyl, halo, halo C₁-C₃alkoxy, halo C₁-C₃alkyl, heteroaryl C₁-C₃alkyl, heterocyclyl, heterocyclyl C₁-C₃alkyl, hydroxy, hydroxy C₁-C₃alkyl, nitro, and oxo; wherein the heteroaryl part of the heteroaryl C₁-C₃alkyl, the heterocyclyl, and the heterocyclyl part of the heterocyclyl C₁-C₃alkyl is further optionally substituted with one, two, or three groups independently selected from C₁-C₃alkoxy, C₁-C₃alkyl, halo, halo C₁-C₃alkyl, and (NR^{x}R^{Y})C₁-C₃alkyl; or
R⁵ and R¹⁶, together with the nitrogen atom to which they are attached, form a heterocyclic group optionally substituted with one, two, three, four, or five groups independently selected from one, two, three, or four groups independently selected from C₁-C₃alkoxy, C₁-C₃alkoxyalkyl, C₁-C₃alkyl, amino, amino C₁-C₃alkyl, hydroxy, and hydroxy C₁-C₃alkyl;
one of R^{a} and R^{b} is selected from hydrogen and C₁-C₃alkyl and the other is selected from hydrogen, C₁-C₃alkyl, C₁-C₃alkoxycarbonyl, C₁-C₃alkylcarbonyl, aryl C₁-C₆alkyl, C₃-C₆cycloalkyl, and C₃-C₆cycloalkyl C₁-C₆alkyl; and
one of R^{x} and R^{y} is hydrogen and the other is selected from —C(O)C₁-C₆alkylheterocyclyl, wherein the heterocycyl is optionally substituted with an oxo group.

In some aspects, the present disclosure provides a compound of formula (I), or a pharmaceutically acceptable salt thereof, wherein:
U is a bond or NH;
Z is a bond, O, NR^{e} or CR^{e}R^{f}, wherein R^{e} and R^{f} are independently hydrogen or C₁-C₃alkyl;
R¹ is aryl or heteroaryl, wherein the aryl and the heteroaryl are optionally substituted with one, two, three, four, or five substituents independently selected from C₁-C₃alkoxy, C₁-C₃alkyl, C₂-C₄alkenyl, C₂-C₄alkynyl, amino, amino C₁-C₃alkyl, cyano, C₃-C₄cycloalkyl, halo, halo C₁-C₃alkyl, hydroxy, and hydroxy C₁-C₃alkyl;
R² and R³ are independently selected from hydrogen, C₁-C₃alkoxy, C₁-C₃alkyl, cyano, halo, halo C₁-C₃alkyl, —C(O)NH₂, —C(O)NH(C₁-C₃alkyl), —C(O)N(C₁-C₃alkyl)₂, and hydroxy;
Y is a bond, O, NR^{g}(CR^{e}R^{f})m, NR^{f}, or CR^{e}R^{f}, wherein m is 1, 2, or 3, and wherein R^{e}, R^{f}, and R^{g} are independently hydrogen or C₁-C₃alkyl;
A is a four- to ten-membered nitrogen-containing monocyclic or bicyclic bridged, fused, or spirocyclic saturated, unsaturated, or partially unsaturated ring system optionally containing one or two heteroatoms independently selected from nitrogen, oxygen, and sulfur, wherein the ring system is optionally substituted with one, two, or three groups independently selected from C₁-C₃alkoxy, C₁-C₃alkoxyalkyl, C₁-C₃alkyl, cyano, cyano C₁-C₃alkyl, halo, halo C₁-C₃alkyl, amino, amino C₁-C₃alkyl, hydroxy, hydroxy C₁-C₃alkyl, and oxo;
R′ is halo;
R⁴ is an aryl or heteroaryl ring; wherein the ring is optionally substituted with one, two, or three substituents independently selected from C₂-C₄alkenyl, C₁-C₃alkoxy, C₁-C₃alkoxy C₁-C₆alkyl, C₁-C₃alkyl, cyano, cyano C₁-C₃alkyl, halo, halo C₁-C₃alkoxy, halo C₁-C₃alkyl, nitro, and oxo;
X is O or NR¹⁶, wherein R¹⁶ is hydrogen or C₁-C₃alkyl;
R⁵ is selected from hydrogen, C₁-C₆alkoxy C₁-C₆alkyl, C₁-C₆alkyl, aryl, aryl C₁-C₆alkyl, carboxy C₁-C₆alkyl, C₃-C₆cycloalkyl, C₃-C₆cycloalkyl C₁-C₆alkyl, di(C₁-C₃alkyl)amino C₂-C₆alkyl, halo C₁-C₆alkyl, heteroaryl, heteroaryl C₁-C₆alkyl, heterocyclyl, heterocyclyl C₁-C₆alkyl, hydroxy C₁-C₆alkyl, NR^{a}R^{b}—C(O)—C₁-C₆alkyl), NR^{a}R^{b} C₁-C₆alkyl, wherein the aryl, the aryl part of the aryl C₁-C₆alkyl, the C₃-C₆cycloalkyl, the cycloalkyl part of the C₃-C₆cycloalkyl C₁-C₆alkyl, the heteroaryl, the heteroaryl part of the heteroaryl C₁-C₆alkyl, the heterocyclyl, the heterocyclyl part of the heterocyclyl C₁-C₆alkyl, are optionally substituted with one, two, three, or four groups independently selected from C₁-C₃alkoxy, C₁-C₃alkyl, (C₁-C₆alkyl)amino, (C₁-C₆alkyl)amino C₁-C₃alkyl, amino, amino C₁-C₃alkyl, carboxy, cyano, di(C₁-C₆alkyl)amino, di(C₁-C₆alkyl)amino C₁-C₃alkyl, halo, halo C₁-C₃alkoxy, halo C₁-C₃alkyl, heterocyclyl, heterocyclyl C₁-C₃alkyl, hydroxy, hydroxy C₁-C₃alkyl, nitro, and oxo; wherein the heterocyclyl, and the heterocyclyl part of the heterocyclyl C₁-C₃alkyl is further optionally substituted with one, two, or three groups independently selected from C₁-C₃alkoxy, C₁-C₃alkyl, halo, and halo C₁-C₃alkyl; or
R⁵ and R¹⁶, together with the nitrogen atom to which they are attached, form a heterocyclic group optionally substituted with one, two, three, four, or five groups independently selected from one, two, three, or four groups independently selected from C₁-C₃alkoxy, C₁-C₃alkoxyalkyl, C₁-C₃alkyl, amino, amino C₁-C₃alkyl, hydroxy, and hydroxy C₁-C₃alkyl; and
one of R^{a} and R^{b} is selected from hydrogen and C₁-C₃alkyl and the other is selected from hydrogen, C₁-C₃alkyl, C₁-C₃alkoxycarbonyl, C₁-C₃alkylcarbonyl, aryl C₁-C₆alkyl, C₃-C₆cycloalkyl, and C₃-C₆cycloalkyl C₁-C₆alkyl.

In some aspects, the present disclosure provides a compound of formula (I), or a pharmaceutically acceptable salt thereof, wherein:
U is a bond or NH;
Z is a bond, O, NR^{e} or CR^{e}R^{f}, wherein R^{e} and R^{f} are independently hydrogen or C₁-C₃alkyl;
R¹ is aryl or heteroaryl, wherein the aryl and the heteroaryl are optionally substituted with one, two, three, four, or five substituents independently selected from C₁-C₃alkoxy, C₁-C₃alkyl, C₂-C₄alkenyl, C₂-C₄alkynyl, amino, amino C₁-C₃alkyl, cyano, C₃-C₄cycloalkyl, halo, halo C₁-C₃alkyl, hydroxy, and hydroxy C₁-C₃alkyl;
R² and R³ are independently selected from hydrogen, C₁-C₃alkoxy, C₁-C₃alkyl, cyano, halo, halo C₁-C₃alkyl, —C(O)NH₂, —C(O)NH(C₁-C₃alkyl), —C(O)N(C₁-C₃alkyl)₂, and hydroxy;
Y is a bond, O, NR^{g}(CR^{e}R^{f})m, NR^{f}, or CR^{e}R^{f}, wherein m is 1, 2, or 3, and wherein R^{e}, R^{f}, and R^{g} are independently hydrogen or C₁-C₃alkyl;
A is a four- to ten-membered nitrogen-containing monocyclic or bicyclic bridged, fused, or spirocyclic saturated, unsaturated, or partially unsaturated ring system optionally containing one or two heteroatoms independently selected from nitrogen, oxygen, and sulfur, wherein the ring system is optionally substituted with one, two, or three groups independently selected from C₁-C₃alkoxy, C₁-C₃alkoxyalkyl, C₁-C₃alkyl, cyano, halo, halo C₁-C₃alkyl, amino, amino C₁-C₃alkyl, hydroxy, hydroxy C₁-C₃alkyl, and oxo;
R′ is halo;
R⁴ is a five- or six-membered aromatic ring optionally containing one, two, or three heteroatoms independently selected from nitrogen, oxygen, and sulfur; wherein the ring is optionally substituted with one, two, or three substituents independently selected from C₂-C₄alkenyl, C₁-C₃alkyl, cyano, cyano C₁-C₃alkyl, halo, halo C₁-C₃alkoxy, halo C₁-C₃alkyl, nitro, and oxo;
X is O or NR¹⁶, wherein R¹⁶ is hydrogen or C₁-C₃alkyl;
R⁵ is selected from hydrogen, C₁-C₆alkoxy C₁-C₆alkyl, C₁-C₆alkyl, aryl, aryl C₁-C₆alkyl, carboxy C₁-C₆alkyl, C₃-C₆cycloalkyl, C₃-C₆cycloalkyl C₁-C₆alkyl, di(C₁-C₃alkyl)amino C₂-C₆alkyl, halo C₁-C₆alkyl, heteroaryl, heteroaryl C₁-C₆alkyl, heterocyclyl, heterocyclyl C₁-C₆alkyl, hydroxy C₁-C₆alkyl, NR^{a}R^{b}—C(O)—C₁-C₆alkyl), NR^{a}R^{b} C₁-C₆alkyl, wherein the aryl, the aryl part of the aryl C₁-C₆alkyl, the C₃-C₆cycloalkyl, the cycloalkyl part of the C₃-C₆cycloalkyl C₁-C₆alkyl, the heteroaryl, the heteroaryl part of the heteroaryl C₁-C₆alkyl, the heterocyclyl, the heterocyclyl part of the heterocyclyl C₁-C₆alkyl, are optionally substituted with one, two, three, or four groups independently selected from C₁-C₃alkoxy, C₁-C₃alkyl, (C₁-C₆alkyl)amino, (C₁-C₆alkyl)amino C₁-C₃alkyl, amino, amino C₁-C₃alkyl, carboxy, cyano, di(C₁-C₆alkyl)amino, di(C₁-C₆alkyl)amino C₁-C₃alkyl, halo, halo C₁-C₃alkoxy, halo C₁-C₃alkyl, heterocyclyl, heterocyclyl C₁-C₃alkyl, hydroxy, hydroxy C₁-C₃alkyl, nitro, and oxo; wherein the heterocyclyl and the heterocyclyl part of the heterocyclyl C₁-C₃alkyl is further optionally substituted with one, two, or three groups independently selected from C₁-C₃alkoxy, C₁-C₃alkyl, halo, and halo C₁-C₃alkyl; or
R⁵ and R¹⁶, together with the nitrogen atom to which they are attached, form a heterocyclic group optionally substituted with one, two, three, four, or five groups independently selected from one, two, three, or four groups independently selected from C₁-C₃alkoxy, C₁-C₃alkoxyalkyl, C₁-C₃alkyl, amino, amino C₁-C₃alkyl, hydroxy, and hydroxy C₁-C₃alkyl; and
one of R^{a} and R^{b} is selected from hydrogen and C₁-C₃alkyl and the other is selected from hydrogen, C₁-C₃alkyl, C₁-C₃alkoxycarbonyl, C₁-C₃alkylcarbonyl, aryl C₁-C₆alkyl, C₃-C₆cycloalkyl, and C₃-C₆cycloalkyl C₁-C₆alkyl.

In some aspects, R⁴ is a five- or six-membered aromatic ring optionally containing one, two, or three heteroatoms independently selected from nitrogen, oxygen, and sulfur.

In some aspects, Y is a bond.

In some aspects, Y is NR. In certain aspects, Y is NCH₃.

In some aspects, A is a four- to nine-membered monocyclic or bicyclic bridged, spirocyclic, or fused saturated ring system optionally containing one or two nitrogen atoms.

In some aspects, A-U is

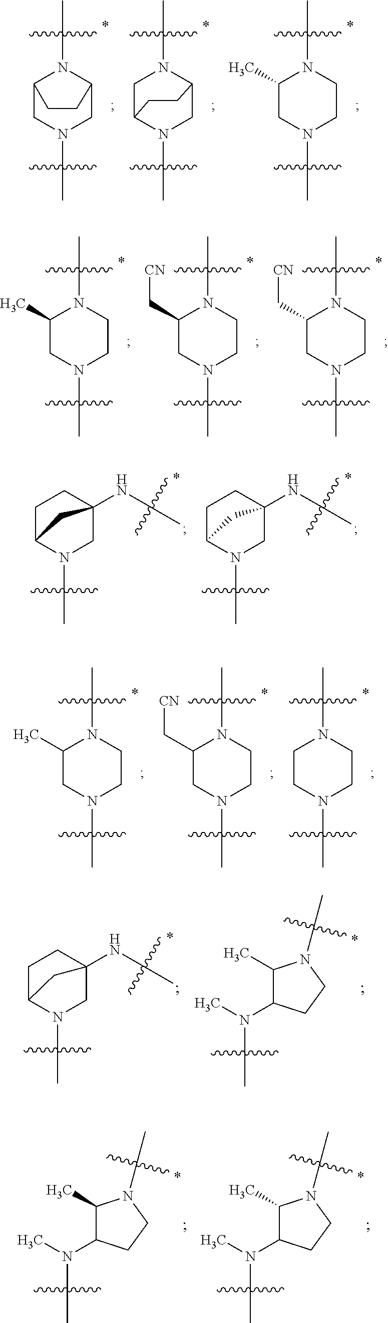

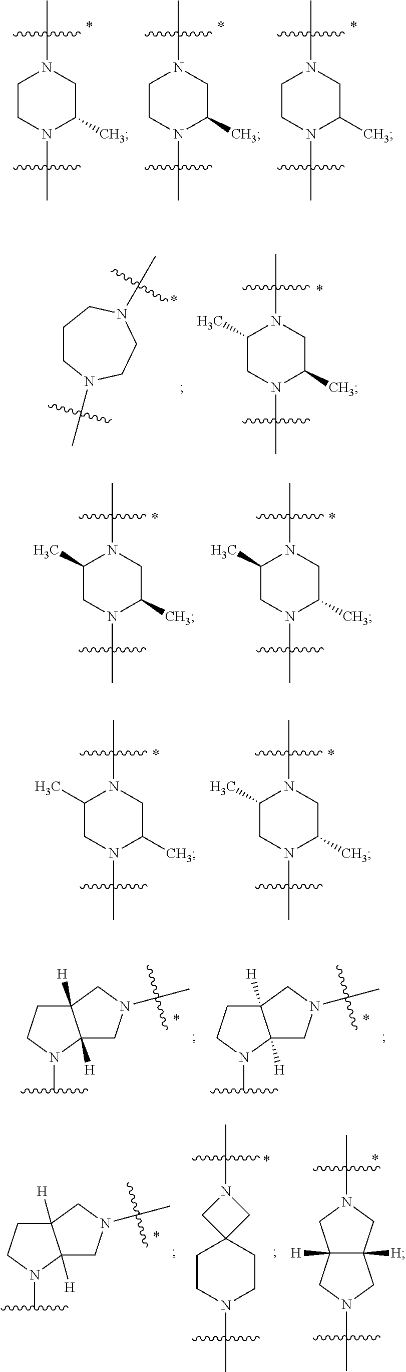

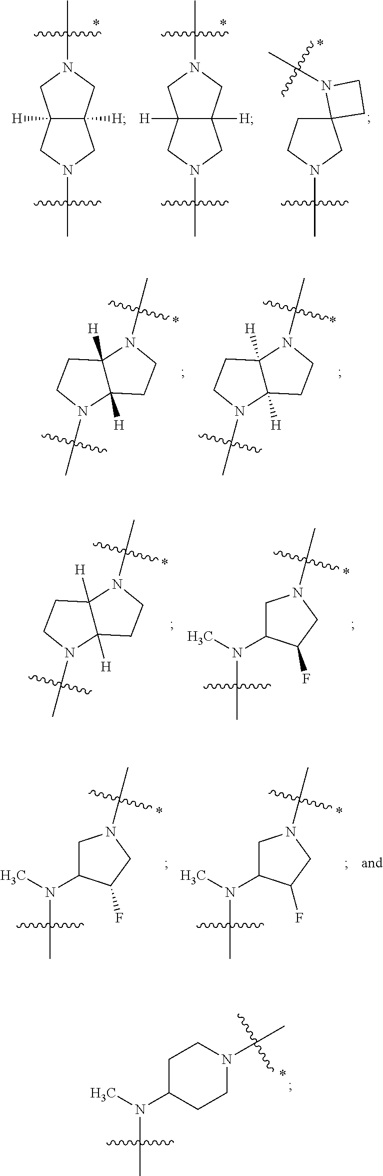

wherein
[symbol] represents the point of attachment to the carbonyl group; and
[symbol] represents the point of attachment to Y.

In some aspects, A-U is

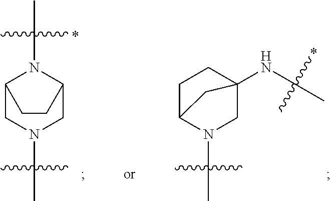 [SMILES: *N1CC2CCC(C1)N2* ; *NC12CCC(C1)N(*)C2]

wherein [symbol] * represents the point of attachment to the carbonyl group; and [symbol] represents the point of attachment to Y.

In some aspects, A-U is

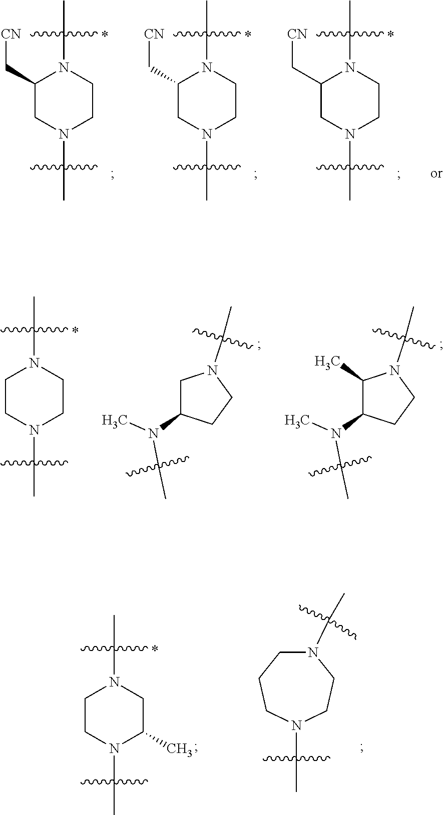

wherein:
[symbol] * represents the point of attachment to the carbonyl group; and
[symbol] represents the point of attachment to Y.

In some aspects, R² is hydrogen or methoxy.

In some aspects, R³ is halo.

In some aspects, R⁴ is selected from imidazolyl, isothiazolyl, isoxazolyl, oxazolyl, phenyl, pyridazinyl, pyridinyl, pyrimidinyl, pyrazolyl, thiazolyl, and triazolyl, wherein each ring is optionally substituted with one, two, or three groups independently selected from C₂-C₄alkenyl, C₁-C₃alkyl, halo, halo C₁-C₃alkoxy, halo C₁-C₃alkyl, nitro, and oxo. In some aspects, R⁴ is selected from imidazolyl, oxazolyl, pyrazinyl, pyridazinyl, pyridinyl, pyrimidinyl, thiadiazolyl, and thiazolyl, wherein each ring is optionally substituted with a methyl or halo.

In some aspects, X is O.

In some aspects, R⁵ is selected from:

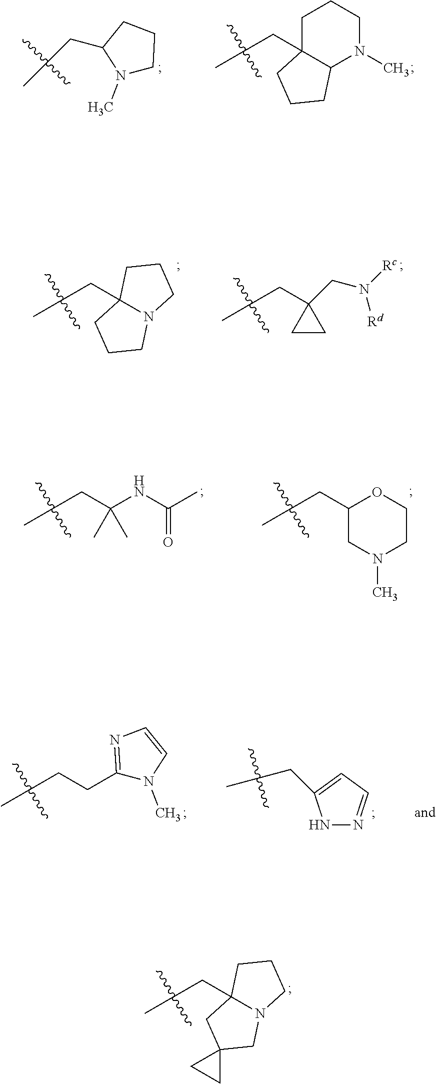

wherein each ring is optionally substituted with 1, 2, or 3 groups independently selected from C₁-C₃alkoxy, C₁-C₃alkoxy C₁-C₃alkyl, C₁-C₃alkyl, benzyl, halo, halo C₁-C₃alkyl, hydroxy, hydroxy C₁-C₃alkyl, and oxo.

In some aspects, R⁵ is —(C₁-C₃alkyl)-R⁶, wherein R⁶ is a three- to five-membered monocyclic ring system, an eight- or nine-membered bicyclic fused saturated ring system, or a ten-membered tricyclic saturated ring system, wherein each ring system optionally contains one nitrogen atom, and wherein each ring system is optionally substituted with one or two groups independently selected from C₁-C₃alkyl, halo, and (4- to 6-membered heterocyclyl)C₁-C₃alkyl; wherein the heterocyclyl part of the (4- to 6-membered heterocyclyl)C₁-C₃alkyl is further optionally substituted with a halo group.

In some aspects, R⁵ is

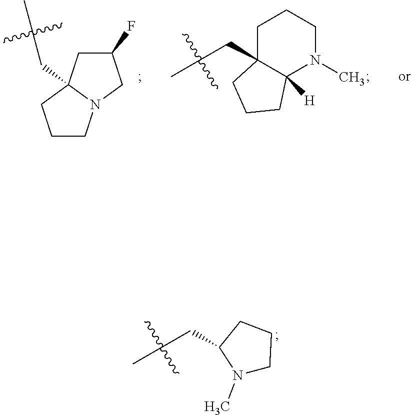

wherein [symbol] represents the point of attachment to X.

In some aspects, R⁵ is

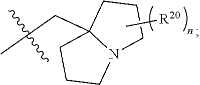

wherein:
n is 0, 1, or 2;
each R²⁰ is halo; and
[symbol] represents the point of attachment to X.

In some aspects, Z is a bond.

In some aspects, R¹ is a monocyclic heteroaryl ring containing one, two, or three nitrogen atoms, wherein the ring is optionally substituted with one, two, three, four, or five substituents independently selected from C₁-C₃alkoxy, C₁-C₃alkyl, C₂-C₄alkenyl, C₂-C₄alkynyl, amino, amino C₁-C₃alkyl, cyano, C₃-C₄cycloalkyl, halo, halo C₁-C₃alkyl, hydroxy, and hydroxy C₁-C₃alkyl.

In some aspects, R¹ is

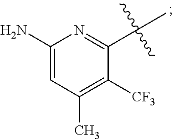

wherein [symbol] denotes the point of attachment to the parent molecular moiety.

In some aspects, R¹ is a bicyclic heteroaryl ring containing one, two, or three heteroatoms selected from nitrogen and sulfur, wherein the ring is optionally substituted with one, two, three, four, or five substituents independently selected from C₁-C₃alkoxy, C₁-C₃alkyl, C₂-C₄alkenyl, C₂-C₄alkynyl, amino, amino C₁-C₃alkyl, cyano, C₃-C₄cycloalkyl, halo, halo C₁-C₃alkyl, hydroxy, and hydroxy C₁-C₃alkyl.

In some aspects, R¹ is a phenyl ring optionally substituted with one, two, three, four, or five substituents independently selected from C₁-C₃alkoxy, C₁-C₃alkyl, C₂-C₄alkenyl, C₂-C₄alkynyl, amino, amino C₁-C₃alkyl, cyano, C₃-C₄cycloalkyl, halo, halo C₁-C₃alkyl, hydroxy, and hydroxy C₁-C₃alkyl.

In some aspects, R¹ is C₆-C₁₀aryl optionally substituted with one, two, three, four, or five substituents independently selected from C₁-C₃alkoxy, C₁-C₃alkyl, C₂-C₄alkenyl, C₂-C₄alkynyl, amino, amino C₁-C₃alkyl, cyano, C₃-C₄cycloalkyl, halo, halo C₁-C₃alkyl, hydroxy, and hydroxy C₁-C₃alkyl. In some aspects, R¹ is naphtyl substituted with one, two, three, four, or five substituents independently selected from C₁-C₃alkyl, C₂-C₄alkynyl, halo, and hydroxy. In some aspects, R¹ is naphthyl, wherein the naphthyl is substituted with one, two, or three groups independently selected from C₂-C₄alkynyl, halo, and hydroxy.

In some aspects, R¹ is

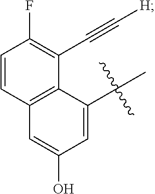

wherein [symbol] denotes the point of attachment to the parent molecular moiety.

In some aspects, R¹ is

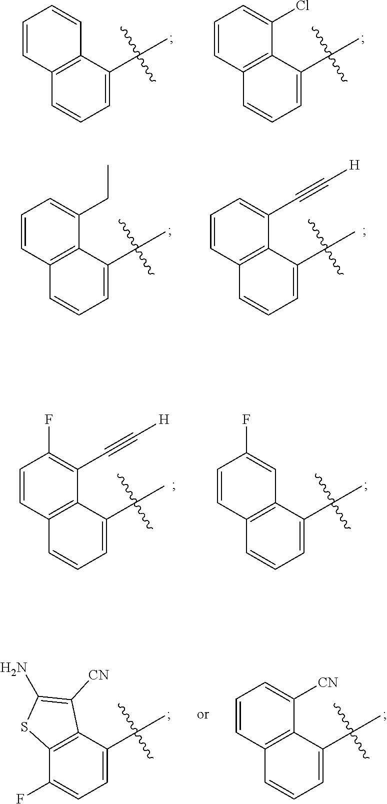

wherein [symbol] denotes the point of attachment to the parent molecular moiety.

In some aspects, R′ is fluoro. In some aspects, R′ is chloro.

In some aspects, the present disclosure provides a compound of formula (IC):

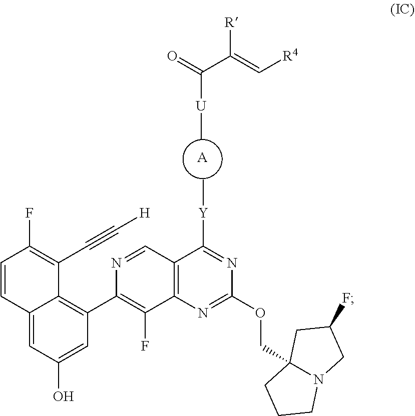

or a pharmaceutically acceptable salt thereof, wherein:
R′ is chloro or fluoro;
A-U is

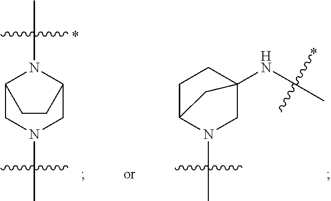 [SMILES: *N1CC2CCC(C1)N2* ; *NC12CCC(C1)N(*)C2]

and
R⁴ is selected from imidazolyl, oxazolyl, pyridazinyl, pyridinyl, pyrimidinyl, and thiazolyl; wherein each ring is optionally substituted with a halo or methyl group.

In some aspects, the present disclosure provides a compound selected from

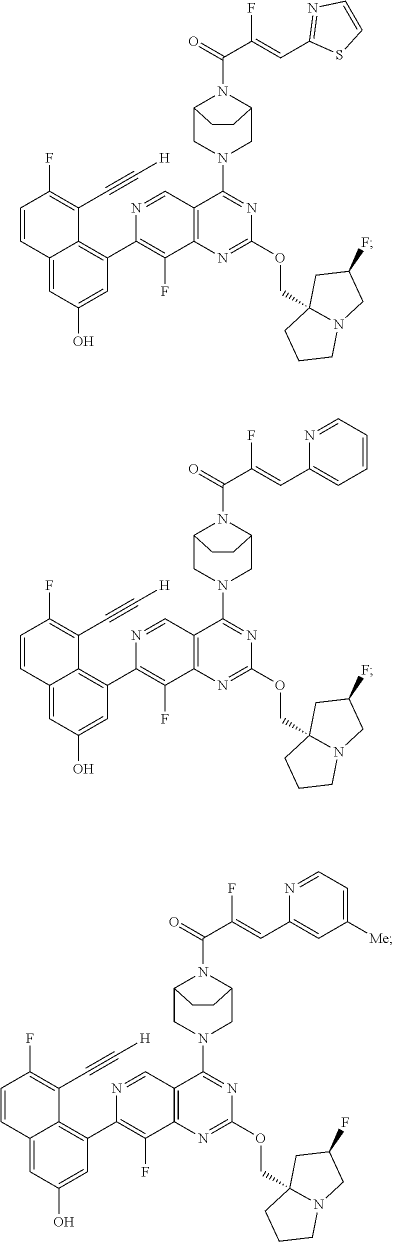 [SMILES: C#Cc1c(F)ccc2cc(O)cc(-c3ncc4c(N5CC6CCC(C5)N6C(=O)/C(F)=C/c5nccs5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5ccccn5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5cc(C)ccn5)nc(OC[C@@]56CCCN5C[C@H](F)C6)nc4c3F)c12]

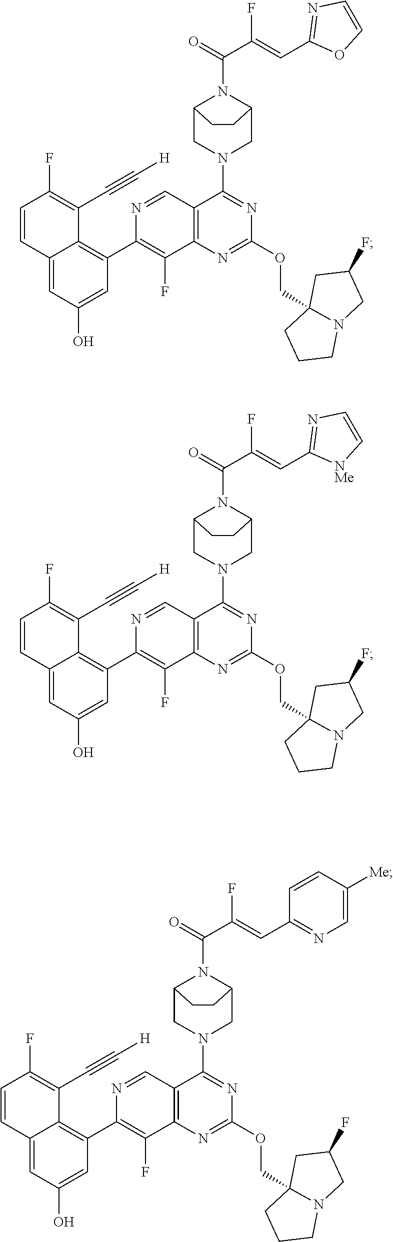 [SMILES: C#Cc1c(F)ccc2cc(O)cc(-c3ncc4c(N5CC6CCC(C5)N6C(=O)/C(F)=C/c5ncco5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5nccn5C)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5ccc(C)cn5)nc(OC[C@@]56CCCN5C[C@H](F)C6)nc4c3F)c12]

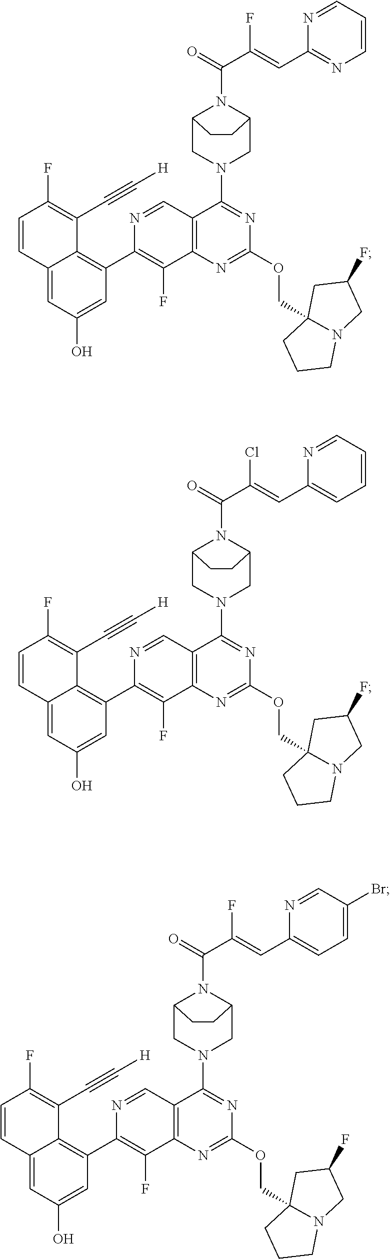 [SMILES: C#Cc1c(F)ccc2cc(O)cc(-c3ncc4c(N5CC6CCC(C5)N6C(=O)/C(F)=C/c5ncccn5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(Cl)=C/c5ccccn5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5ccc(Br)cn5)nc(OC[C@@]56CCCN5C[C@H](F)C6)nc4c3F)c12]

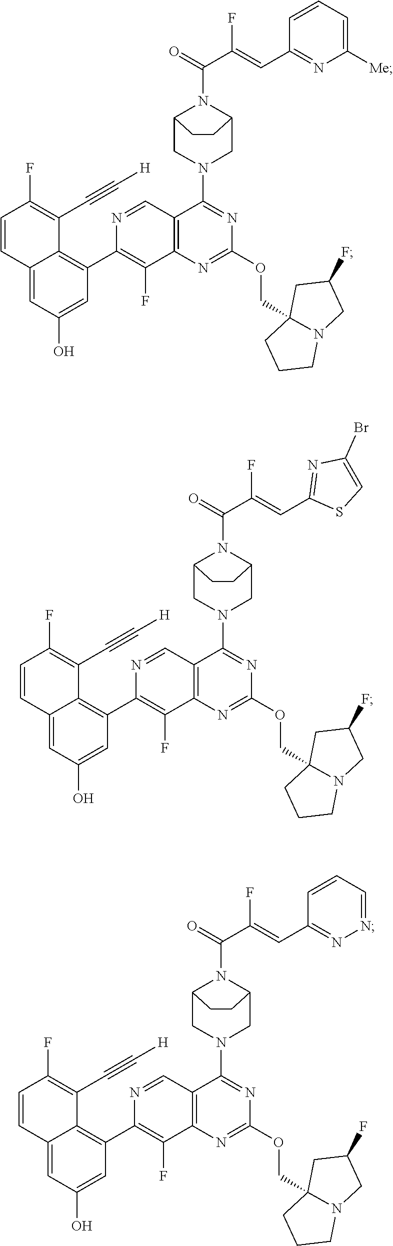 [SMILES: C#Cc1c(F)ccc2cc(O)cc(-c3ncc4c(N5CC6CCC(C5)N6C(=O)/C(F)=C/c5cccc(C)n5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5nc(Br)cs5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5cccnn5)nc(OC[C@@]56CCCN5C[C@H](F)C6)nc4c3F)c12]

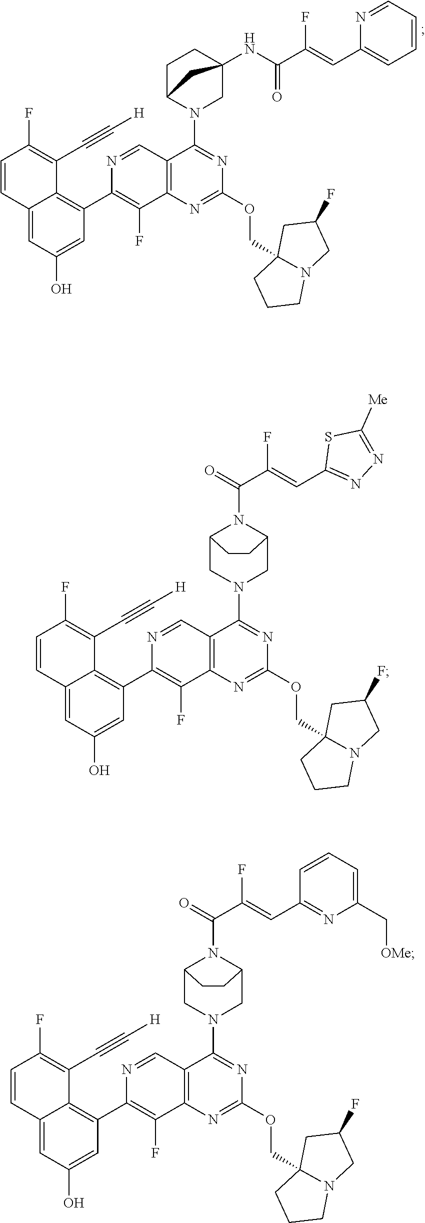 [SMILES: C#Cc1c(F)ccc2cc(O)cc(-c3ncc4c(N5C[C@@]6(NC(=O)/C(F)=C/c7ccccn7)CC[C@@H]5C6)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5nnc(C)s5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5cccc(COC)n5)nc(OC[C@@]56CCCN5C[C@H](F)C6)nc4c3F)c12]

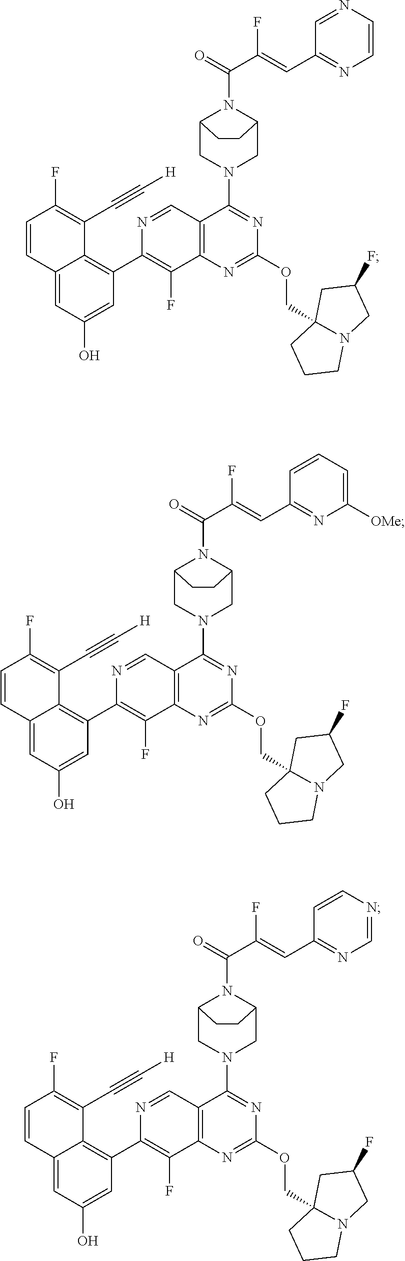 [SMILES: C#Cc1c(F)ccc2cc(O)cc(-c3ncc4c(N5CC6CCC(C5)N6C(=O)/C(F)=C/c5cnccn5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5cccc(OC)n5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5ccncn5)nc(OC[C@@]56CCCN5C[C@H](F)C6)nc4c3F)c12]

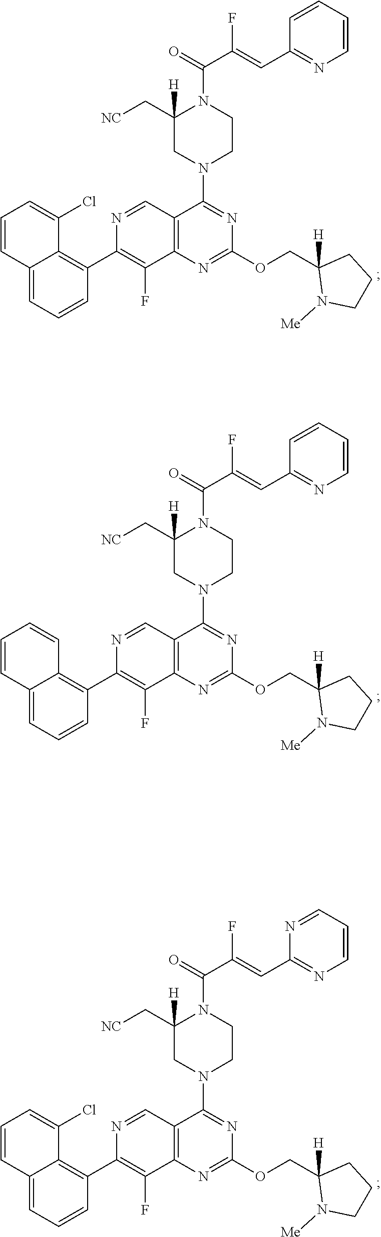 [SMILES: CN1CCC[C@H]1COc1nc(N2CCN(C(=O)/C(F)=C/c3ccccn3)[C@@H](CC#N)C2)c2cnc(-c3cccc4cccc(Cl)c34)c(F)c2n1 ; CN1CCC[C@H]1COc1nc(N2CCN(C(=O)/C(F)=C/c3ccccn3)[C@@H](CC#N)C2)c2cnc(-c3cccc4ccccc34)c(F)c2n1 ; CN1CCC[C@H]1COc1nc(N2CCN(C(=O)/C(F)=C/c3ncccn3)[C@@H](CC#N)C2)c2cnc(-c3cccc4cccc(Cl)c34)c(F)c2n1]

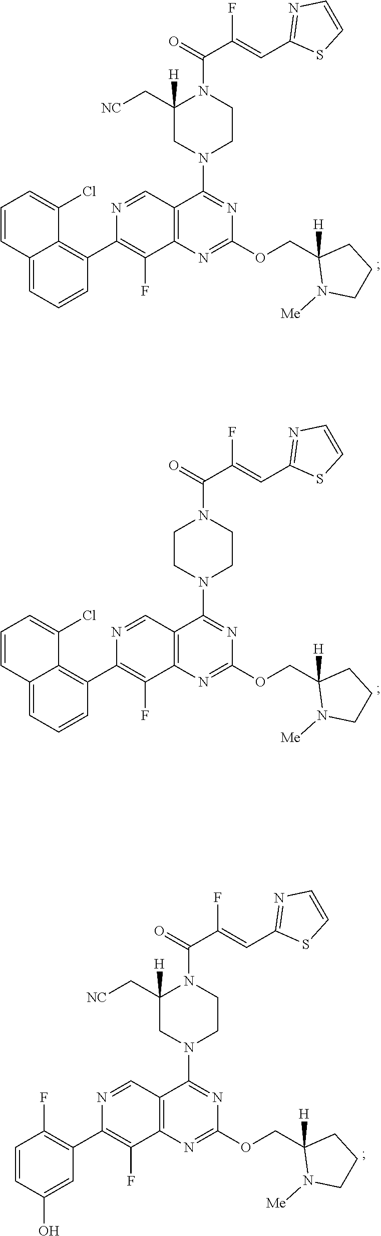 [SMILES: CN1CCC[C@H]1COc1nc(N2CCN(C(=O)/C(F)=C/c3nccs3)[C@@H](CC#N)C2)c2cnc(-c3cccc4cccc(Cl)c34)c(F)c2n1 ; CN1CCC[C@H]1COc1nc(N2CCN(C(=O)/C(F)=C/c3nccs3)CC2)c2cnc(-c3cccc4cccc(Cl)c34)c(F)c2n1 ; CN1CCC[C@H]1COc1nc(N2CCN(C(=O)/C(F)=C/c3nccs3)[C@@H](CC#N)C2)c2cnc(-c3cc(O)ccc3F)c(F)c2n1]

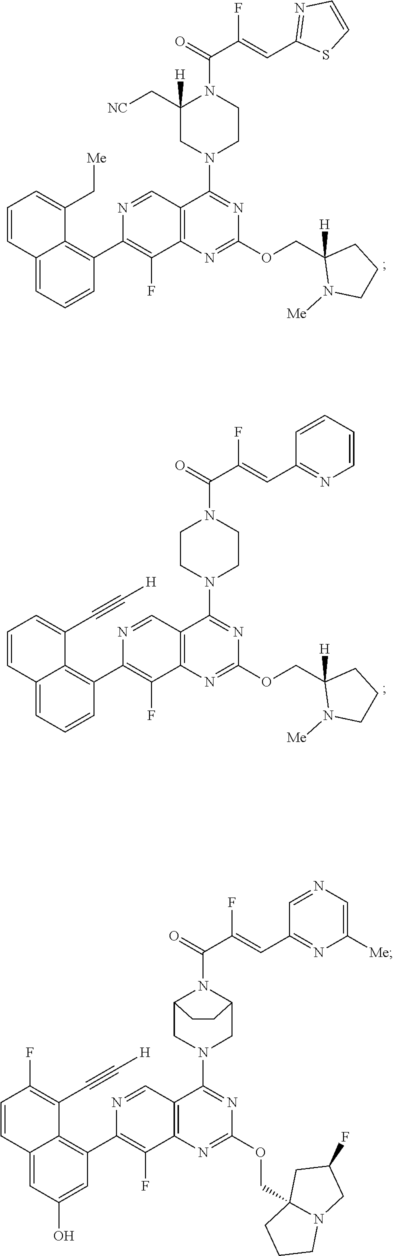 [SMILES: CCc1cccc2cccc(-c3ncc4c(N5CCN(C(=O)/C(F)=C/c6nccs6)[C@@H](CC#N)C5)nc(OC[C@@H]5CCCN5C)nc4c3F)c12 ; C#Cc1cccc2cccc(-c3ncc4c(N5CCN(C(=O)/C(F)=C/c6ccccn6)CC5)nc(OC[C@@H]5CCCN5C)nc4c3F)c12 ; C#Cc1c(F)ccc2cc(O)cc(-c3ncc4c(N5CC6CCC(C5)N6C(=O)/C(F)=C/c5cncc(C)n5)nc(OC[C@@]56CCCN5C[C@H](F)C6)nc4c3F)c12]

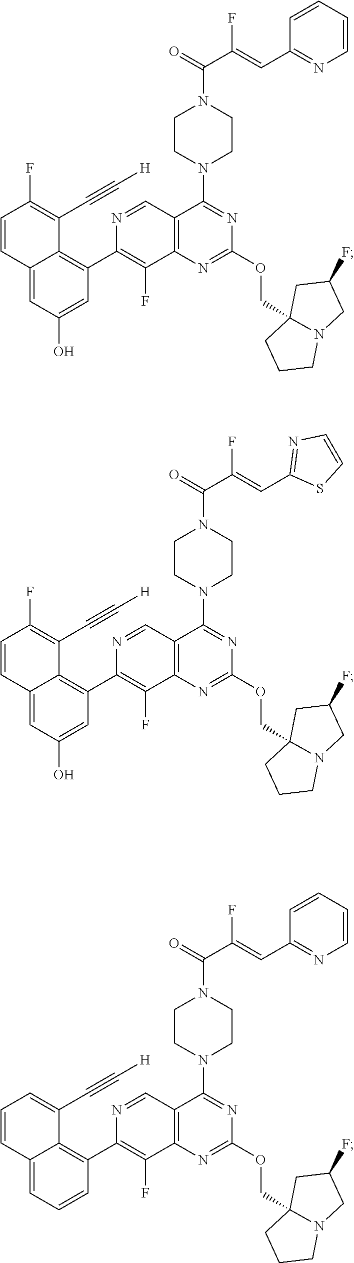 [SMILES: C#Cc1c(F)ccc2cc(O)cc(-c3ncc4c(N5CCN(C(=O)/C(F)=C/c6ccccn6)CC5)nc(OC[C@@]56CCCN5C[C@H](F)C6)nc4c3F)c12 ; C#Cc1c(F)ccc2cc(O)cc(-c3ncc4c(N5CCN(C(=O)/C(F)=C/c6nccs6)CC5)nc(OC[C@@]56CCCN5C[C@H](F)C6)nc4c3F)c12 ; C#Cc1cccc2cccc(-c3ncc4c(N5CCN(C(=O)/C(F)=C/c6ccccn6)CC5)nc(OC[C@@]56CCCN5C[C@H](F)C6)nc4c3F)c12]

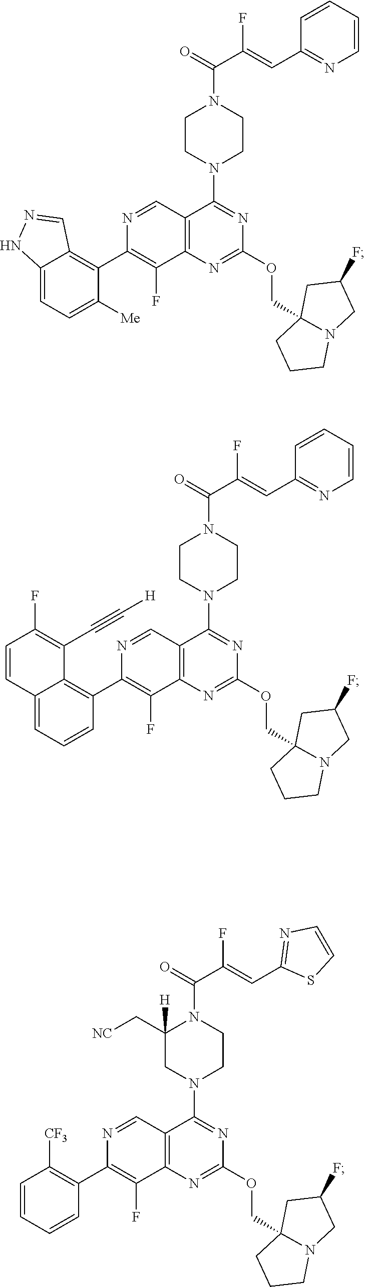 [SMILES: Cc1ccc2[nH]ncc2c1-c1ncc2c(N3CCN(C(=O)/C(F)=C/c4ccccn4)CC3)nc(OC[C@@]34CCCN3C[C@H](F)C4)nc2c1F ; C#Cc1c(F)ccc2cccc(-c3ncc4c(N5CCN(C(=O)/C(F)=C/c6ccccn6)CC5)nc(OC[C@@]56CCCN5C[C@H](F)C6)nc4c3F)c12 ; N#CC[C@H]1CN(c2nc(OC[C@@]34CCCN3C[C@H](F)C4)nc3c(F)c(-c4ccccc4C(F)(F)F)ncc23)CCN1C(=O)/C(F)=C/c1nccs1]

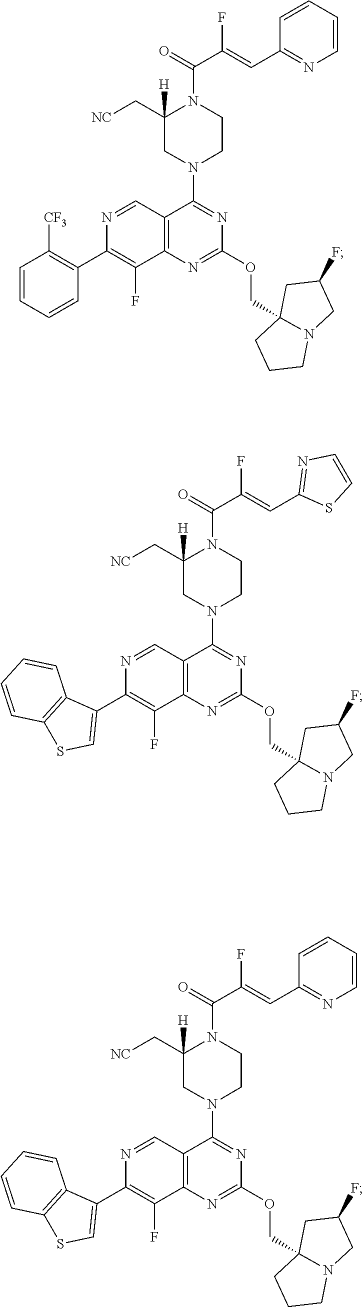 [SMILES: N#CC[C@H]1CN(c2nc(OC[C@@]34CCCN3C[C@H](F)C4)nc3c(F)c(-c4ccccc4C(F)(F)F)ncc23)CCN1C(=O)/C(F)=C/c1ccccn1 ; N#CC[C@H]1CN(c2nc(OC[C@@]34CCCN3C[C@H](F)C4)nc3c(F)c(-c4csc5ccccc45)ncc23)CCN1C(=O)/C(F)=C/c1nccs1 ; N#CC[C@H]1CN(c2nc(OC[C@@]34CCCN3C[C@H](F)C4)nc3c(F)c(-c4csc5ccccc45)ncc23)CCN1C(=O)/C(F)=C/c1ccccn1]

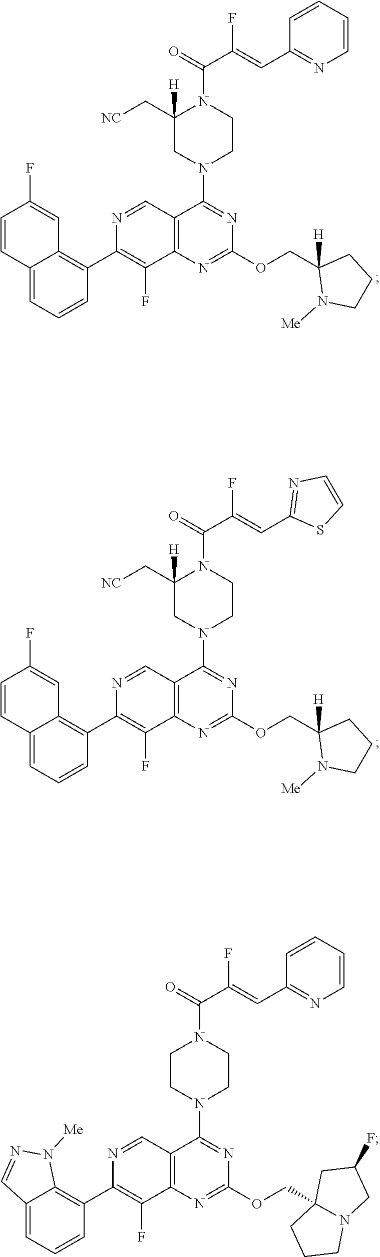 [SMILES: CN1CCC[C@H]1COc1nc(N2CCN(C(=O)/C(F)=C/c3ccccn3)[C@@H](CC#N)C2)c2cnc(-c3cccc4ccc(F)cc34)c(F)c2n1 ; CN1CCC[C@H]1COc1nc(N2CCN(C(=O)/C(F)=C/c3nccs3)[C@@H](CC#N)C2)c2cnc(-c3cccc4ccc(F)cc34)c(F)c2n1 ; Cn1ncc2cccc(-c3ncc4c(N5CCN(C(=O)/C(F)=C/c6ccccn6)CC5)nc(OC[C@@]56CCCN5C[C@H](F)C6)nc4c3F)c21]

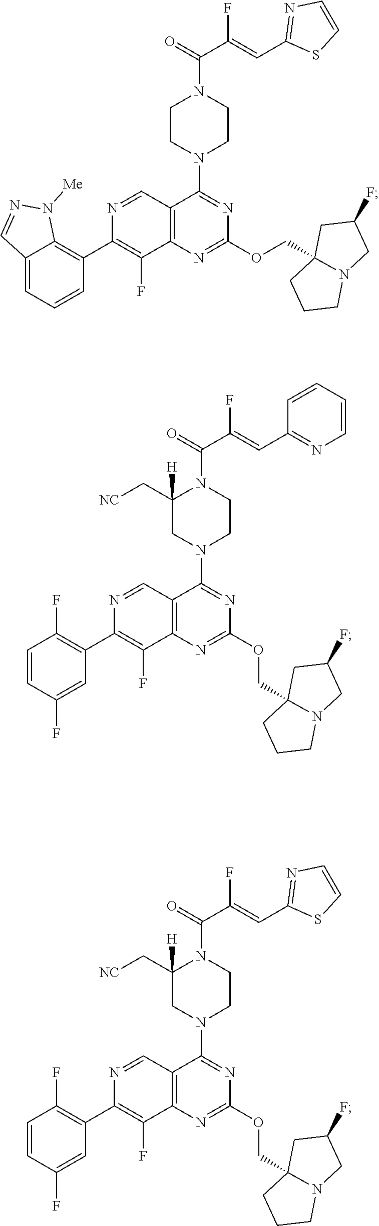 [SMILES: Cn1ncc2cccc(-c3ncc4c(N5CCN(C(=O)/C(F)=C/c6nccs6)CC5)nc(OC[C@@]56CCCN5C[C@H](F)C6)nc4c3F)c21 ; N#CC[C@H]1CN(c2nc(OC[C@@]34CCCN3C[C@H](F)C4)nc3c(F)c(-c4cc(F)ccc4F)ncc23)CCN1C(=O)/C(F)=C/c1ccccn1 ; N#CC[C@H]1CN(c2nc(OC[C@@]34CCCN3C[C@H](F)C4)nc3c(F)c(-c4cc(F)ccc4F)ncc23)CCN1C(=O)/C(F)=C/c1nccs1]

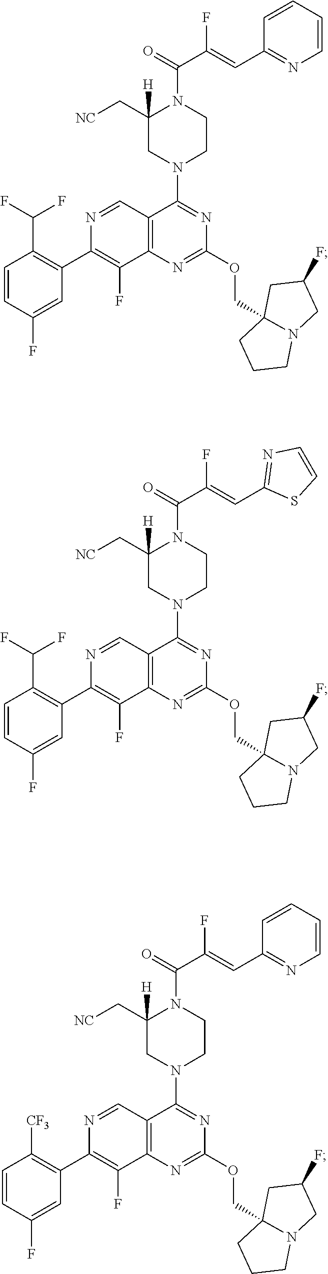 [SMILES: N#CC[C@H]1CN(c2nc(OC[C@@]34CCCN3C[C@H](F)C4)nc3c(F)c(-c4cc(F)ccc4C(F)F)ncc23)CCN1C(=O)/C(F)=C/c1ccccn1 ; N#CC[C@H]1CN(c2nc(OC[C@@]34CCCN3C[C@H](F)C4)nc3c(F)c(-c4cc(F)ccc4C(F)F)ncc23)CCN1C(=O)/C(F)=C/c1nccs1 ; N#CC[C@H]1CN(c2nc(OC[C@@]34CCCN3C[C@H](F)C4)nc3c(F)c(-c4cc(F)ccc4C(F)(F)F)ncc23)CCN1C(=O)/C(F)=C/c1ccccn1]

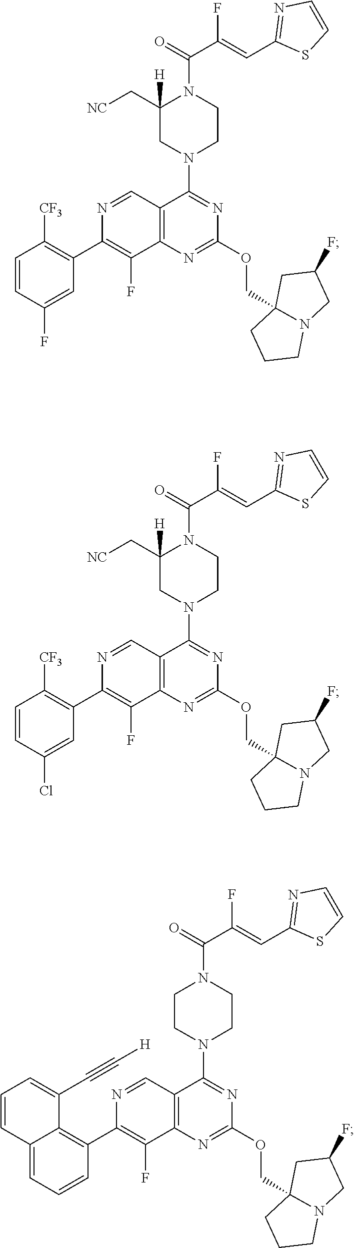 [SMILES: N#CC[C@H]1CN(c2nc(OC[C@@]34CCCN3C[C@H](F)C4)nc3c(F)c(-c4cc(F)ccc4C(F)(F)F)ncc23)CCN1C(=O)/C(F)=C/c1nccs1 ; N#CC[C@H]1CN(c2nc(OC[C@@]34CCCN3C[C@H](F)C4)nc3c(F)c(-c4cc(Cl)ccc4C(F)(F)F)ncc23)CCN1C(=O)/C(F)=C/c1nccs1 ; C#Cc1cccc2cccc(-c3ncc4c(N5CCN(C(=O)/C(F)=C/c6nccs6)CC5)nc(OC[C@@]56CCCN5C[C@H](F)C6)nc4c3F)c12]

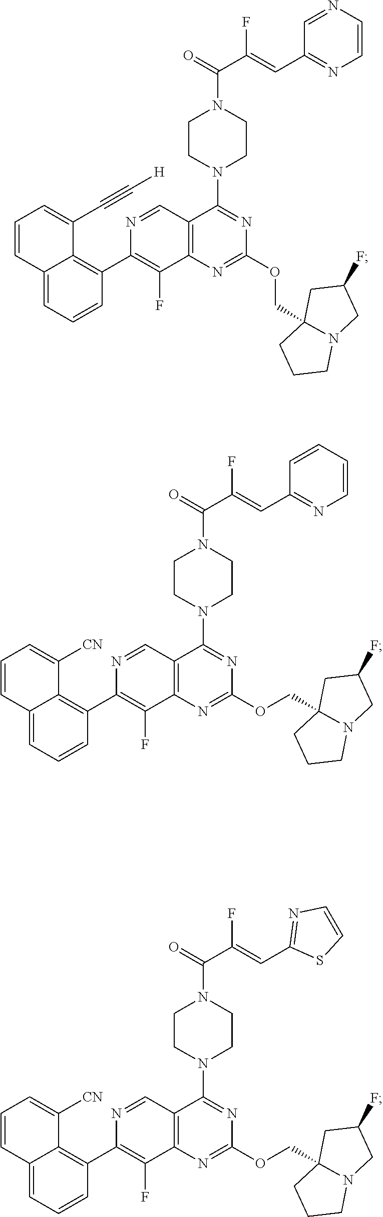 [SMILES: C#Cc1cccc2cccc(-c3ncc4c(N5CCN(C(=O)/C(F)=C/c6cnccn6)CC5)nc(OC[C@@]56CCCN5C[C@H](F)C6)nc4c3F)c12 ; N#Cc1cccc2cccc(-c3ncc4c(N5CCN(C(=O)/C(F)=C/c6ccccn6)CC5)nc(OC[C@@]56CCCN5C[C@H](F)C6)nc4c3F)c12 ; N#Cc1cccc2cccc(-c3ncc4c(N5CCN(C(=O)/C(F)=C/c6nccs6)CC5)nc(OC[C@@]56CCCN5C[C@H](F)C6)nc4c3F)c12]

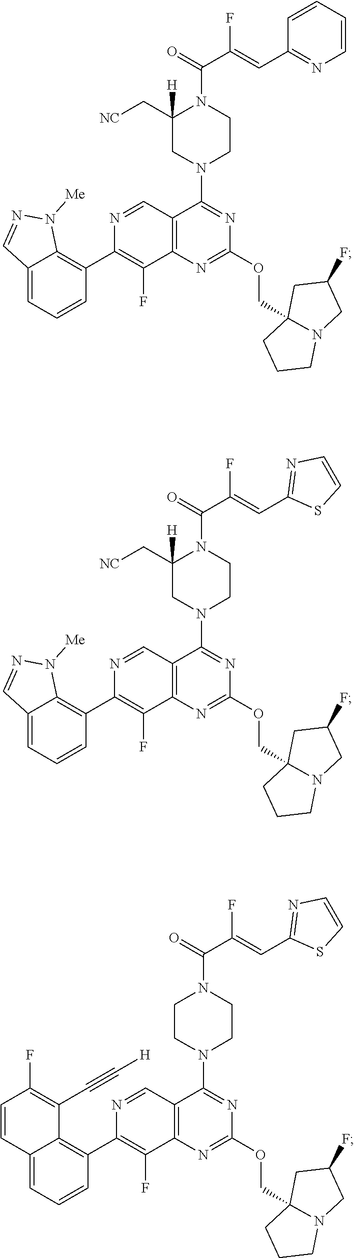 [SMILES: Cn1ncc2cccc(-c3ncc4c(N5CCN(C(=O)/C(F)=C/c6ccccn6)[C@@H](CC#N)C5)nc(OC[C@@]56CCCN5C[C@H](F)C6)nc4c3F)c21 ; Cn1ncc2cccc(-c3ncc4c(N5CCN(C(=O)/C(F)=C/c6nccs6)[C@@H](CC#N)C5)nc(OC[C@@]56CCCN5C[C@H](F)C6)nc4c3F)c21 ; C#Cc1c(F)ccc2cccc(-c3ncc4c(N5CCN(C(=O)/C(F)=C/c6nccs6)CC5)nc(OC[C@@]56CCCN5C[C@H](F)C6)nc4c3F)c12]

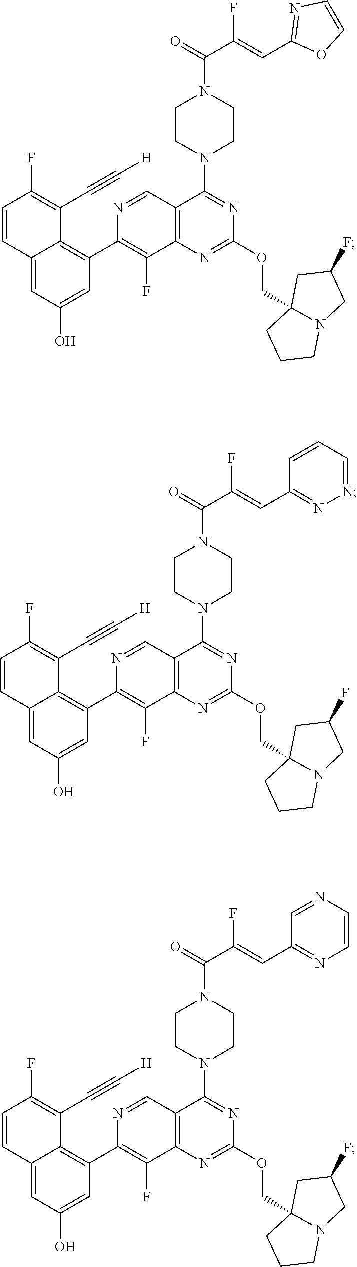 [SMILES: C#Cc1c(F)ccc2cc(O)cc(-c3ncc4c(N5CCN(C(=O)/C(F)=C/c6ncco6)CC5)nc(OC[C@@]56CCCN5C[C@H](F)C6)nc4c3F)c12 ; C#Cc1c(F)ccc2cc(O)cc(-c3ncc4c(N5CCN(C(=O)/C(F)=C/c6cccnn6)CC5)nc(OC[C@@]56CCCN5C[C@H](F)C6)nc4c3F)c12 ; C#Cc1c(F)ccc2cc(O)cc(-c3ncc4c(N5CCN(C(=O)/C(F)=C/c6cnccn6)CC5)nc(OC[C@@]56CCCN5C[C@H](F)C6)nc4c3F)c12]

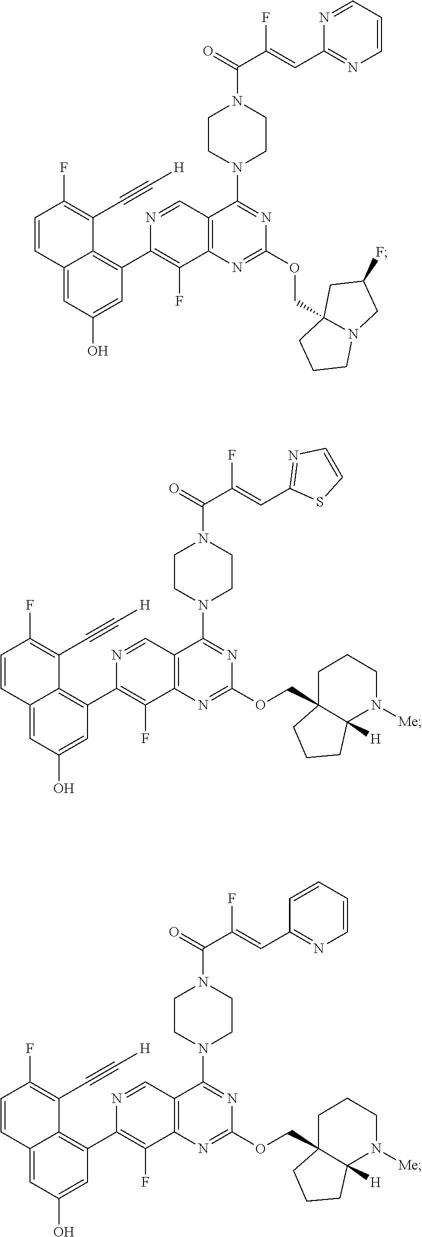 [SMILES: C#Cc1c(F)ccc2cc(O)cc(-c3ncc4c(N5CCN(C(=O)/C(F)=C/c6ncccn6)CC5)nc(OC[C@@]56CCCN5C[C@H](F)C6)nc4c3F)c12 ; C#Cc1c(F)ccc2cc(O)cc(-c3ncc4c(N5CCN(C(=O)/C(F)=C/c6nccs6)CC5)nc(OC[C@]56CCC[C@H]5N(C)CCC6)nc4c3F)c12 ; C#Cc1c(F)ccc2cc(O)cc(-c3ncc4c(N5CCN(C(=O)/C(F)=C/c6ccccn6)CC5)nc(OC[C@]56CCC[C@H]5N(C)CCC6)nc4c3F)c12]

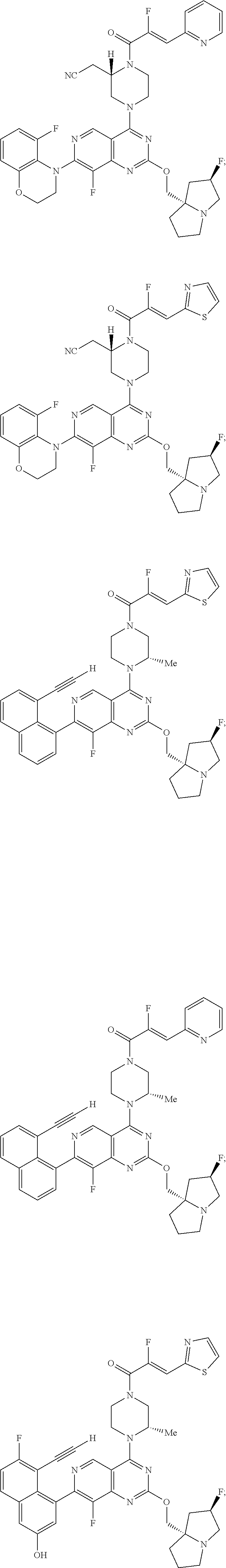

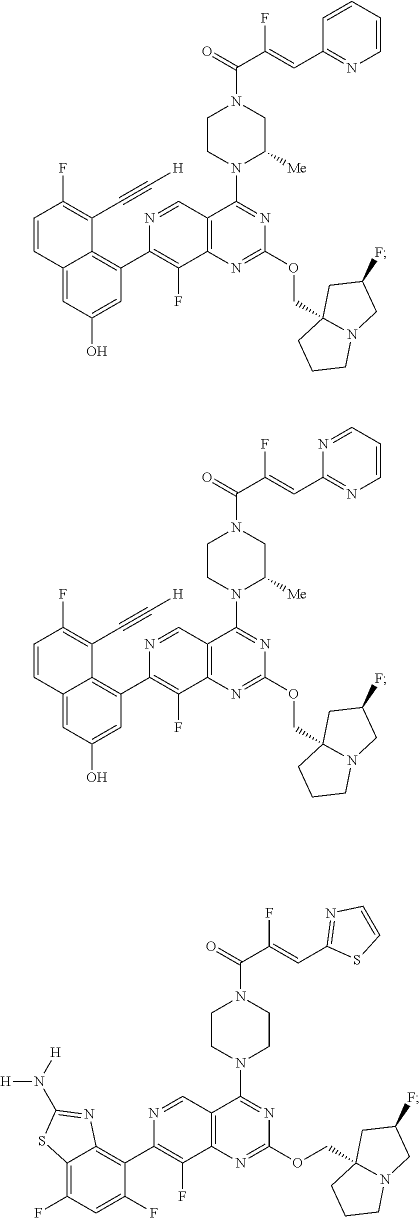 [SMILES: C#Cc1c(F)ccc2cc(O)cc(-c3ncc4c(N5CCN(C(=O)/C(F)=C/c6ccccn6)C[C@@H]5C)nc(OC[C@@]56CCCN5C[C@H](F)C6)nc4c3F)c12 ; C#Cc1c(F)ccc2cc(O)cc(-c3ncc4c(N5CCN(C(=O)/C(F)=C/c6ncccn6)C[C@@H]5C)nc(OC[C@@]56CCCN5C[C@H](F)C6)nc4c3F)c12 ; Nc1nc2c(-c3ncc4c(N5CCN(C(=O)/C(F)=C/c6nccs6)CC5)nc(OC[C@@]56CCCN5C[C@H](F)C6)nc4c3F)c(F)cc(F)c2s1]

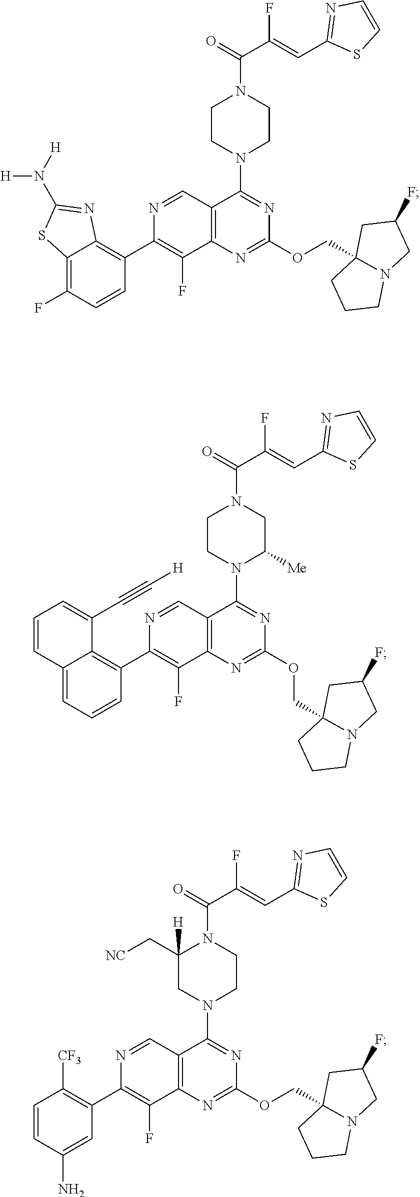 [SMILES: Nc1nc2c(-c3ncc4c(N5CCN(C(=O)/C(F)=C/c6nccs6)CC5)nc(OC[C@@]56CCCN5C[C@H](F)C6)nc4c3F)ccc(F)c2s1 ; C#Cc1cccc2cccc(-c3ncc4c(N5CCN(C(=O)/C(F)=C/c6nccs6)C[C@@H]5C)nc(OC[C@@]56CCCN5C[C@H](F)C6)nc4c3F)c12 ; N#CC[C@H]1CN(c2nc(OC[C@@]34CCCN3C[C@H](F)C4)nc3c(F)c(-c4cc(N)ccc4C(F)(F)F)ncc23)CCN1C(=O)/C(F)=C/c1nccs1]

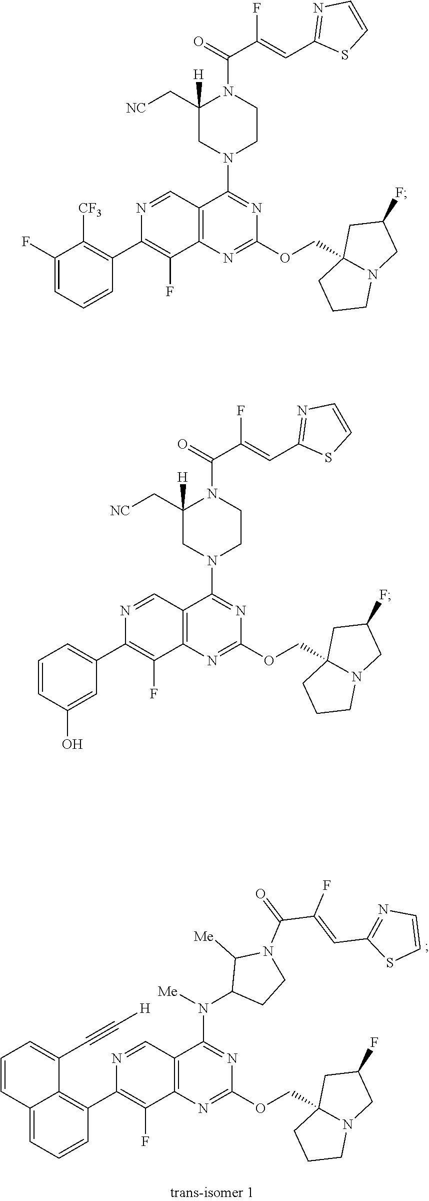 [SMILES: N#CC[C@H]1CN(c2nc(OC[C@@]34CCCN3C[C@H](F)C4)nc3c(F)c(-c4cccc(F)c4C(F)(F)F)ncc23)CCN1C(=O)/C(F)=C/c1nccs1 ; N#CC[C@H]1CN(c2nc(OC[C@@]34CCCN3C[C@H](F)C4)nc3c(F)c(-c4cccc(O)c4)ncc23)CCN1C(=O)/C(F)=C/c1nccs1 ; C#Cc1cccc2cccc(-c3ncc4c(N(C)C5CCN(C(=O)/C(F)=C/c6nccs6)C5C)nc(OC[C@@]56CCCN5C[C@H](F)C6)nc4c3F)c12]

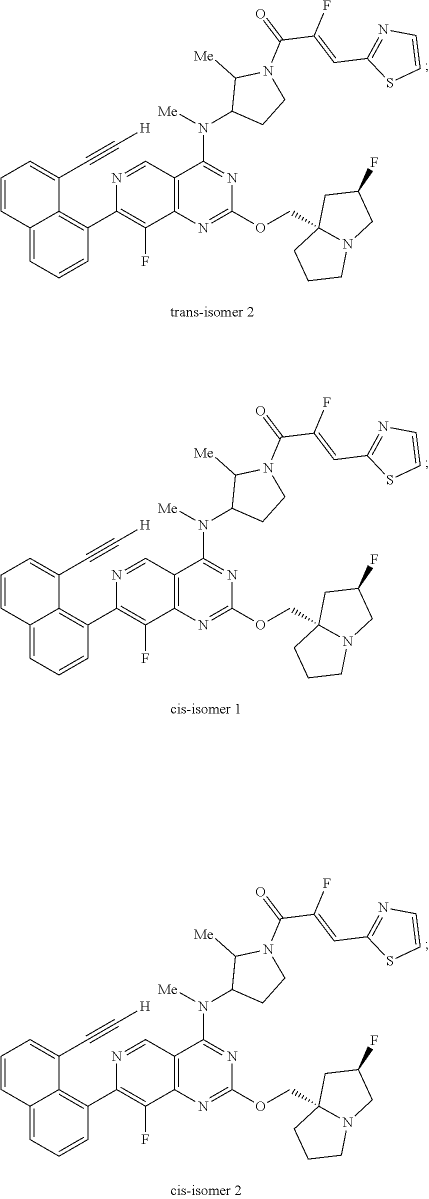 [SMILES: C#Cc1cccc2cccc(-c3ncc4c(N(C)C5CCN(C(=O)/C(F)=C/c6nccs6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6nccs6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6nccs6)C5C)nc(OC[C@@]56CCCN5C[C@H](F)C6)nc4c3F)c12]

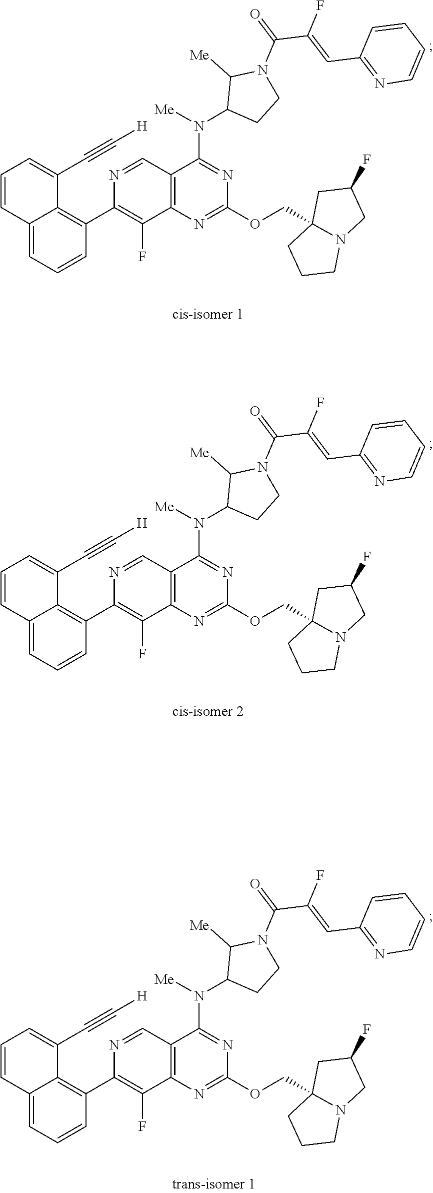 [SMILES: C#Cc1cccc2cccc(-c3ncc4c(N(C)C5CCN(C(=O)/C(F)=C/c6ccccn6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6ccccn6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6ccccn6)C5C)nc(OC[C@@]56CCCN5C[C@H](F)C6)nc4c3F)c12]

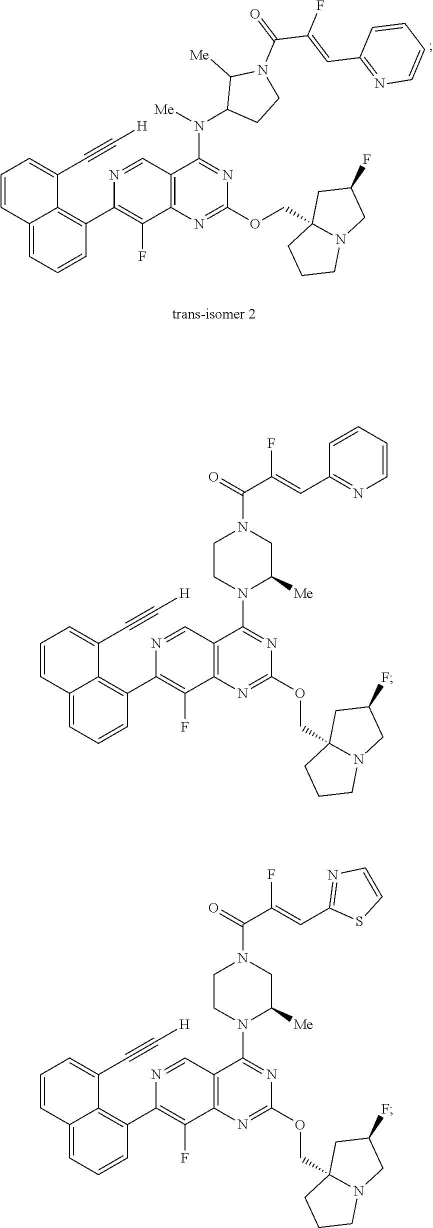 [SMILES: C#Cc1cccc2cccc(-c3ncc4c(N(C)C5CCN(C(=O)/C(F)=C/c6ccccn6)C5C)nc(OC[C@@]56CCCN5C[C@H](F)C6)nc4c3F)c12 ; C#Cc1cccc2cccc(-c3ncc4c(N5CCN(C(=O)/C(F)=C/c6ccccn6)C[C@H]5C)nc(OC[C@@]56CCCN5C[C@H](F)C6)nc4c3F)c12 ; C#Cc1cccc2cccc(-c3ncc4c(N5CCN(C(=O)/C(F)=C/c6nccs6)C[C@H]5C)nc(OC[C@@]56CCCN5C[C@H](F)C6)nc4c3F)c12]

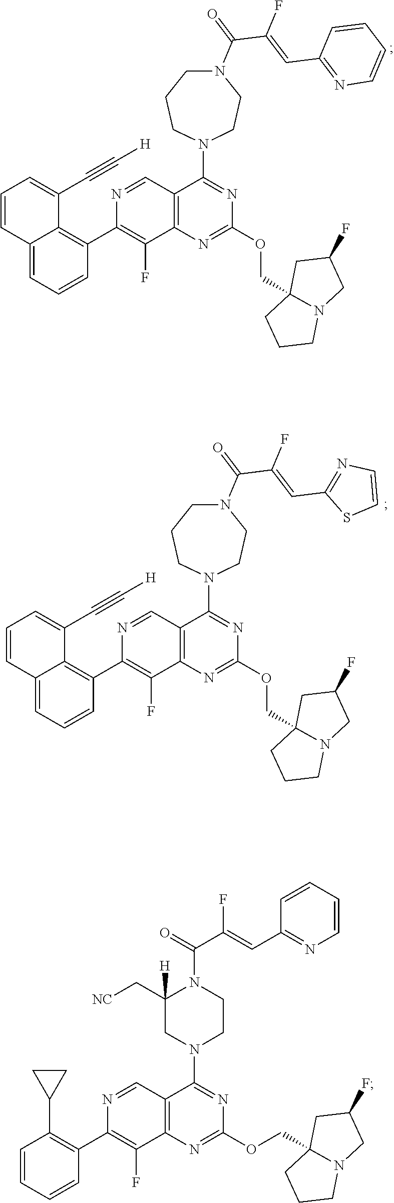 [SMILES: C#Cc1cccc2cccc(-c3ncc4c(N5CCCN(C(=O)/C(F)=C/c6ccccn6)CC5)nc(OC[C@@]56CCCN5C[C@H](F)C6)nc4c3F)c12 ; C#Cc1cccc2cccc(-c3ncc4c(N5CCCN(C(=O)/C(F)=C/c6nccs6)CC5)nc(OC[C@@]56CCCN5C[C@H](F)C6)nc4c3F)c12 ; N#CC[C@H]1CN(c2nc(OC[C@@]34CCCN3C[C@H](F)C4)nc3c(F)c(-c4ccccc4C4CC4)ncc23)CCN1C(=O)/C(F)=C/c1ccccn1]

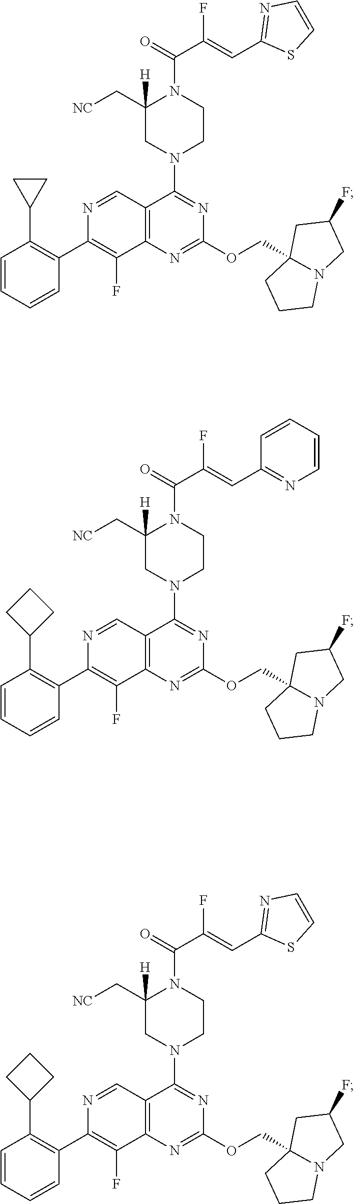 [SMILES: N#CC[C@H]1CN(c2nc(OC[C@@]34CCCN3C[C@H](F)C4)nc3c(F)c(-c4ccccc4C4CC4)ncc23)CCN1C(=O)/C(F)=C/c1nccs1 ; N#CC[C@H]1CN(c2nc(OC[C@@]34CCCN3C[C@H](F)C4)nc3c(F)c(-c4ccccc4C4CCC4)ncc23)CCN1C(=O)/C(F)=C/c1ccccn1 ; N#CC[C@H]1CN(c2nc(OC[C@@]34CCCN3C[C@H](F)C4)nc3c(F)c(-c4ccccc4C4CCC4)ncc23)CCN1C(=O)/C(F)=C/c1nccs1]

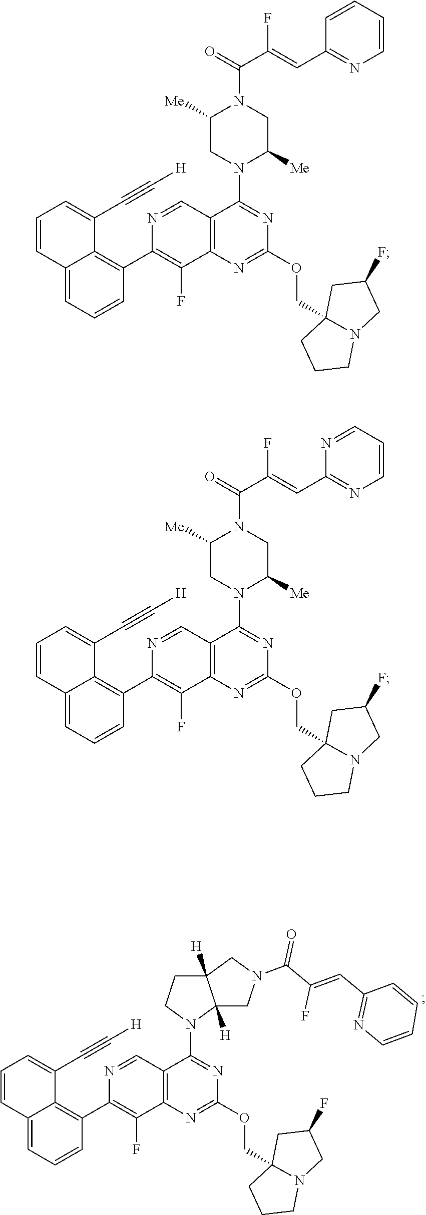 [SMILES: C#Cc1cccc2cccc(-c3ncc4c(N5C[C@H](C)N(C(=O)/C(F)=C/c6ccccn6)C[C@H]5C)nc(OC[C@@]56CCCN5C[C@H](F)C6)nc4c3F)c12 ; C#Cc1cccc2cccc(-c3ncc4c(N5C[C@H](C)N(C(=O)/C(F)=C/c6ncccn6)C[C@H]5C)nc(OC[C@@]56CCCN5C[C@H](F)C6)nc4c3F)c12 ; C#Cc1cccc2cccc(-c3ncc4c(N5CC[C@@H]6CN(C(=O)/C(F)=C/c7ccccn7)C[C@@H]65)nc(OC[C@@]56CCCN5C[C@H](F)C6)nc4c3F)c12]

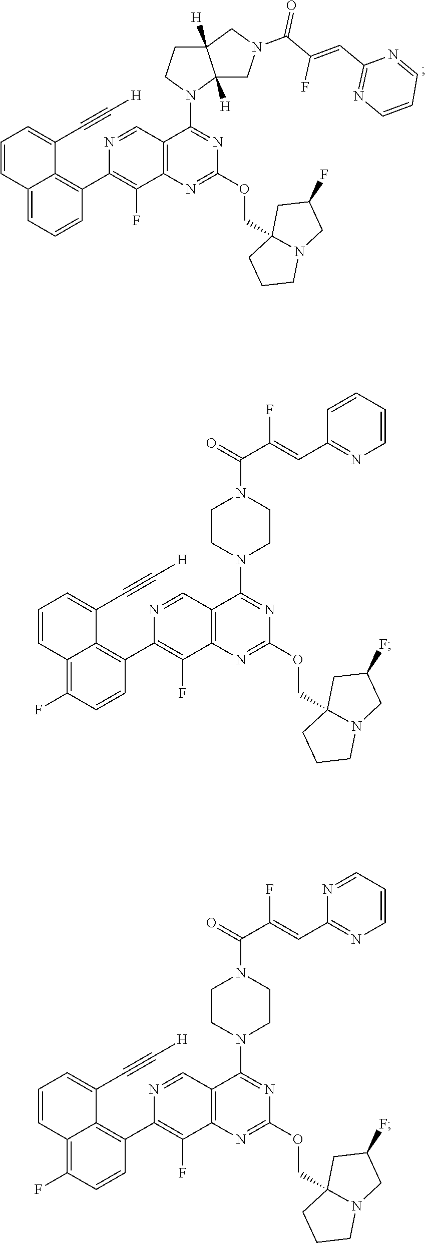 [SMILES: C#Cc1cccc2cccc(-c3ncc4c(N5CC[C@@H]6CN(C(=O)/C(F)=C/c7ncccn7)C[C@@H]65)nc(OC[C@@]56CCCN5C[C@H](F)C6)nc4c3F)c12 ; C#Cc1cccc2c(F)ccc(-c3ncc4c(N5CCN(C(=O)/C(F)=C/c6ccccn6)CC5)nc(OC[C@@]56CCCN5C[C@H](F)C6)nc4c3F)c12 ; C#Cc1cccc2c(F)ccc(-c3ncc4c(N5CCN(C(=O)/C(F)=C/c6ncccn6)CC5)nc(OC[C@@]56CCCN5C[C@H](F)C6)nc4c3F)c12]

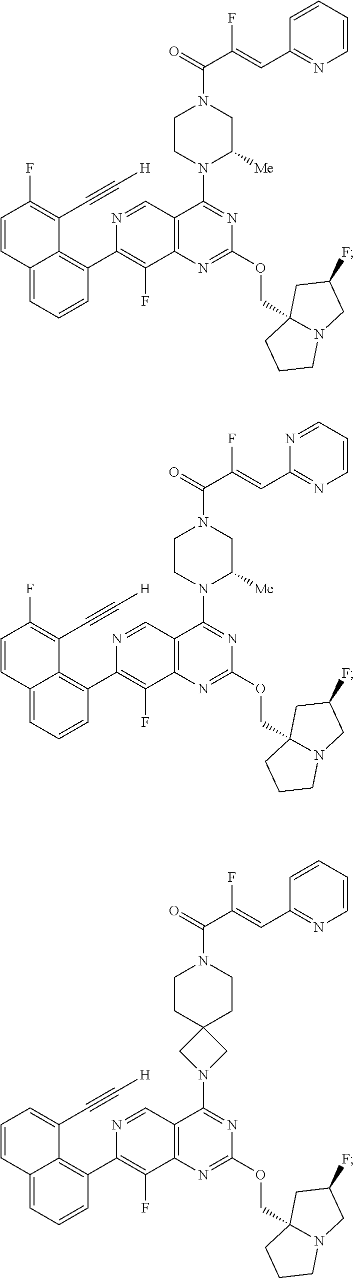 [SMILES: C#Cc1c(F)ccc2cccc(-c3ncc4c(N5CCN(C(=O)/C(F)=C/c6ccccn6)C[C@@H]5C)nc(OC[C@@]56CCCN5C[C@H](F)C6)nc4c3F)c12 ; C#Cc1c(F)ccc2cccc(-c3ncc4c(N5CCN(C(=O)/C(F)=C/c6ncccn6)C[C@@H]5C)nc(OC[C@@]56CCCN5C[C@H](F)C6)nc4c3F)c12 ; C#Cc1cccc2cccc(-c3ncc4c(N5CC6(CCN(C(=O)/C(F)=C/c7ccccn7)CC6)C5)nc(OC[C@@]56CCCN5C[C@H](F)C6)nc4c3F)c12]

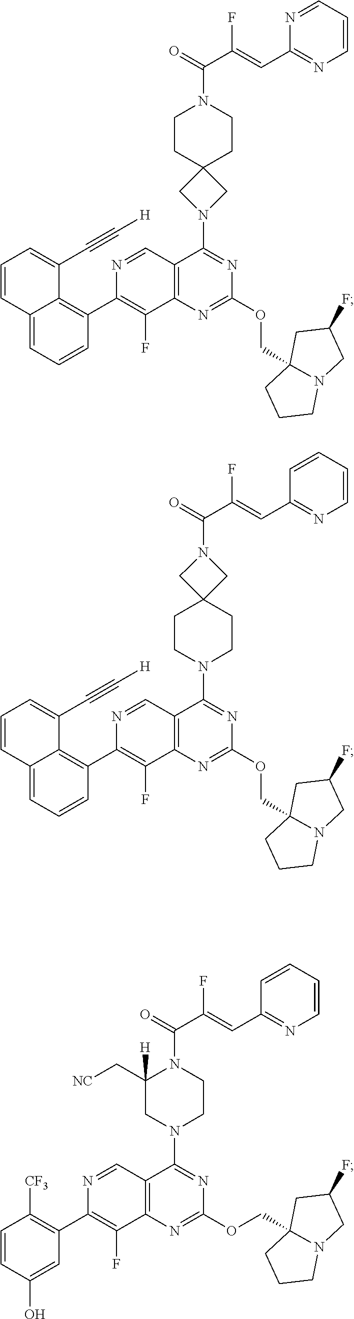

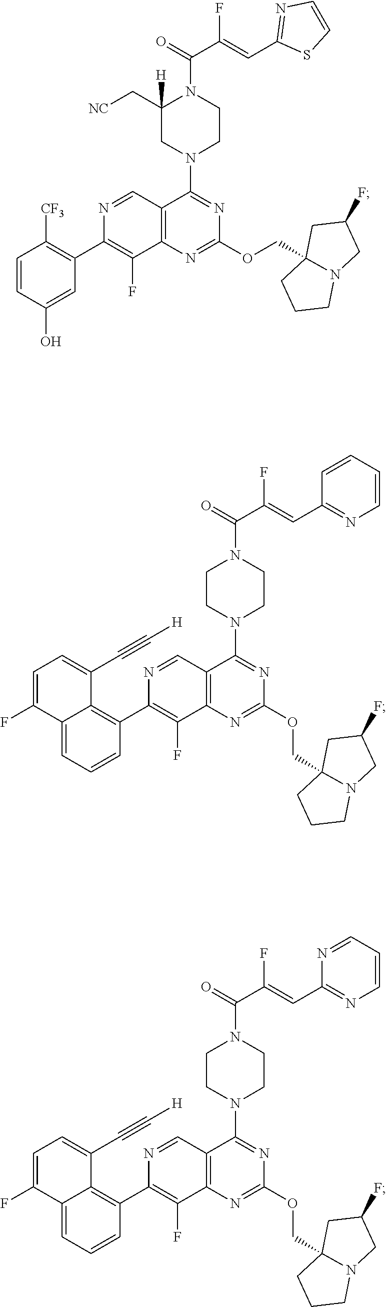 [SMILES: N#CC[C@H]1CN(c2nc(OC[C@@]34CCCN3C[C@H](F)C4)nc3c(F)c(-c4cc(O)ccc4C(F)(F)F)ncc23)CCN1C(=O)/C(F)=C/c1nccs1 ; C#Cc1ccc(F)c2cccc(-c3ncc4c(N5CCN(C(=O)/C(F)=C/c6ccccn6)CC5)nc(OC[C@@]56CCCN5C[C@H](F)C6)nc4c3F)c12 ; C#Cc1ccc(F)c2cccc(-c3ncc4c(N5CCN(C(=O)/C(F)=C/c6ncccn6)CC5)nc(OC[C@@]56CCCN5C[C@H](F)C6)nc4c3F)c12]

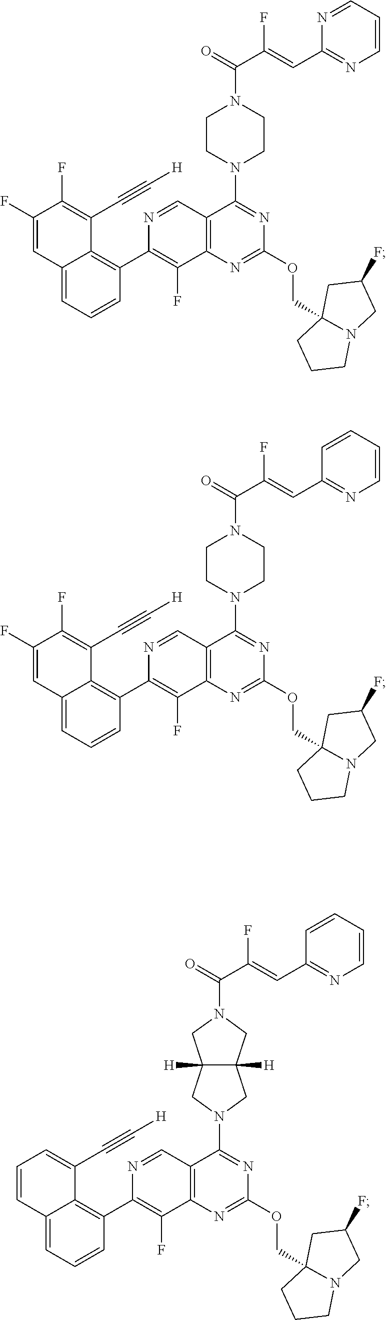 [SMILES: C#Cc1c(F)c(F)cc2cccc(-c3ncc4c(N5CCN(C(=O)/C(F)=C/c6ncccn6)CC5)nc(OC[C@@]56CCCN5C[C@H](F)C6)nc4c3F)c12 ; C#Cc1c(F)c(F)cc2cccc(-c3ncc4c(N5CCN(C(=O)/C(F)=C/c6ccccn6)CC5)nc(OC[C@@]56CCCN5C[C@H](F)C6)nc4c3F)c12 ; C#Cc1cccc2cccc(-c3ncc4c(N5C[C@H]6CN(C(=O)/C(F)=C/c7ccccn7)C[C@H]6C5)nc(OC[C@@]56CCCN5C[C@H](F)C6)nc4c3F)c12]

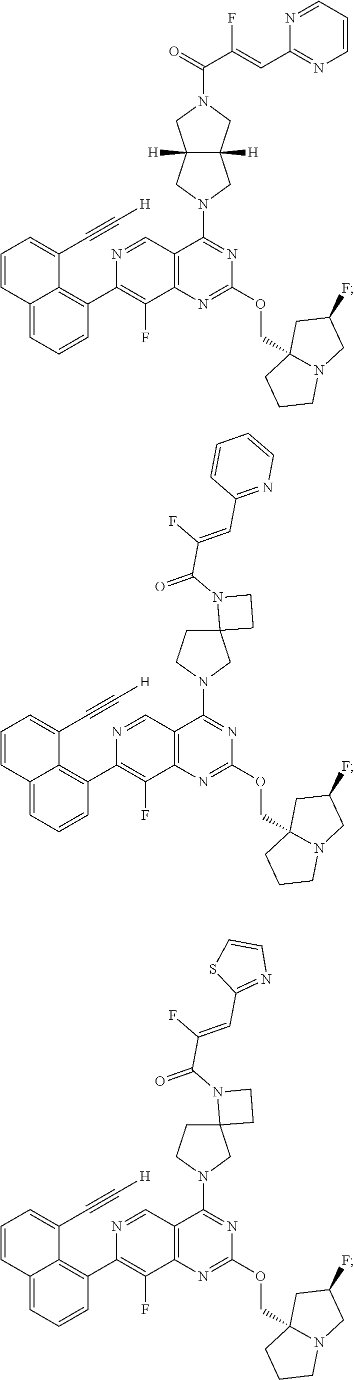

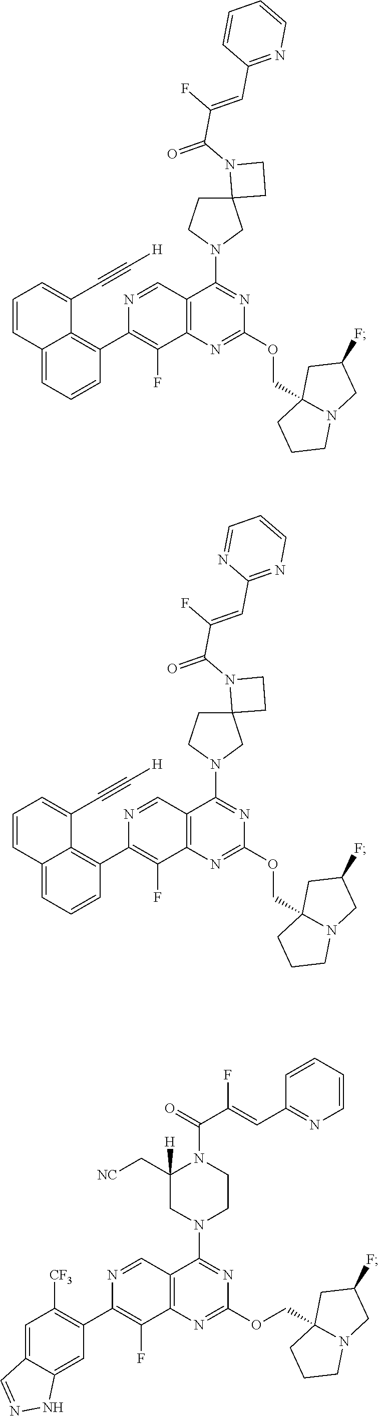 [SMILES: C#Cc1cccc2cccc(-c3ncc4c(N5CCC6(CCN6C(=O)/C(F)=C/c6ccccn6)C5)nc(OC[C@@]56CCCN5C[C@H](F)C6)nc4c3F)c12 ; C#Cc1cccc2cccc(-c3ncc4c(N5CCC6(CCN6C(=O)/C(F)=C/c6ncccn6)C5)nc(OC[C@@]56CCCN5C[C@H](F)C6)nc4c3F)c12 ; N#CC[C@H]1CN(c2nc(OC[C@@]34CCCN3C[C@H](F)C4)nc3c(F)c(-c4cc5[nH]ncc5cc4C(F)(F)F)ncc23)CCN1C(=O)/C(F)=C/c1ccccn1]

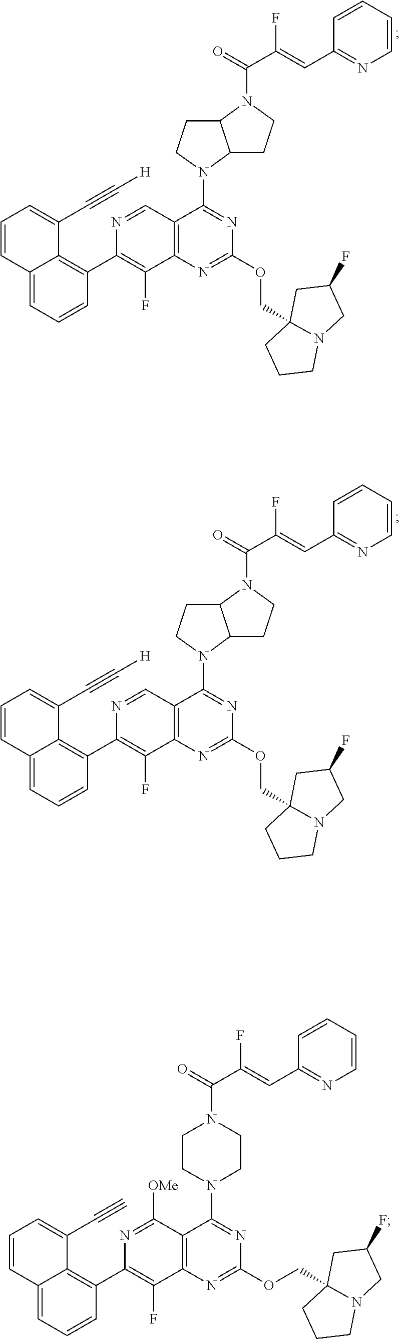 [SMILES: C#Cc1cccc2cccc(-c3ncc4c(N5CCC6C5CCN6C(=O)/C(F)=C/c5ccccn5)nc(OC[C@@]56CCCN5C[C@H](F)C6)nc4c3F)c12 ; C#Cc1cccc2cccc(-c3ncc4c(N5CCC6C5CCN6C(=O)/C(F)=C/c5ccccn5)nc(OC[C@@]56CCCN5C[C@H](F)C6)nc4c3F)c12 ; C#Cc1cccc2cccc(-c3nc(OC)c4c(N5CCN(C(=O)/C(F)=C/c6ccccn6)CC5)nc(OC[C@@]56CCCN5C[C@H](F)C6)nc4c3F)c12]

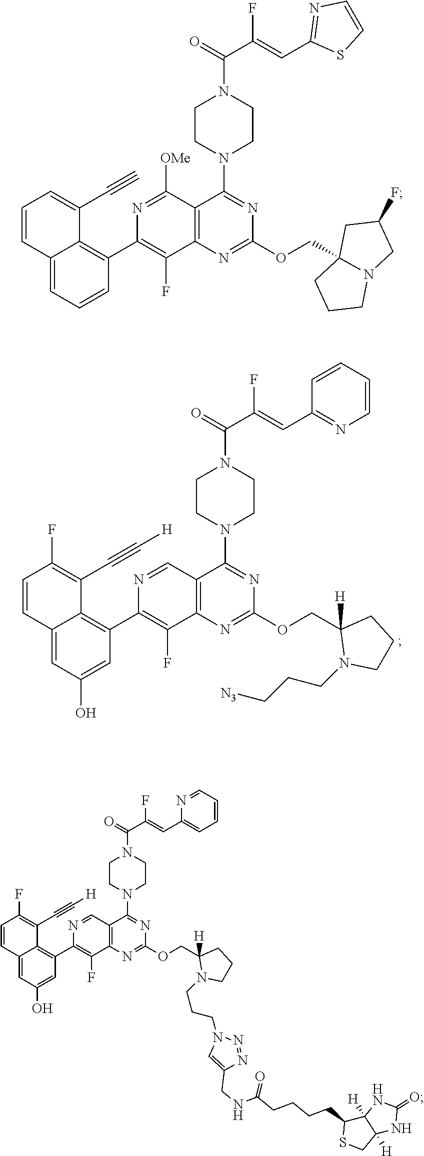 [SMILES: C#Cc1cccc2cccc(-c3nc(OC)c4c(N5CCN(C(=O)/C(F)=C/c6nccs6)CC5)nc(OC[C@@]56CCCN5C[C@H](F)C6)nc4c3F)c12 ; C#Cc1c(F)ccc2cc(O)cc(-c3ncc4c(N5CCN(C(=O)/C(F)=C/c6ccccn6)CC5)nc(OC[C@@H]5CCCN5CCCN=[N+]=[N-])nc4c3F)c12 ; C#Cc1c(F)ccc2cc(O)cc(-c3ncc4c(N5CCN(C(=O)/C(F)=C/c6ccccn6)CC5)nc(OC[C@@H]5CCCN5CCCn5cc(CNC(=O)CCCC[C@@H]6SC[C@@H]7NC(=O)N[C@@H]76)nn5)nc4c3F)c12]

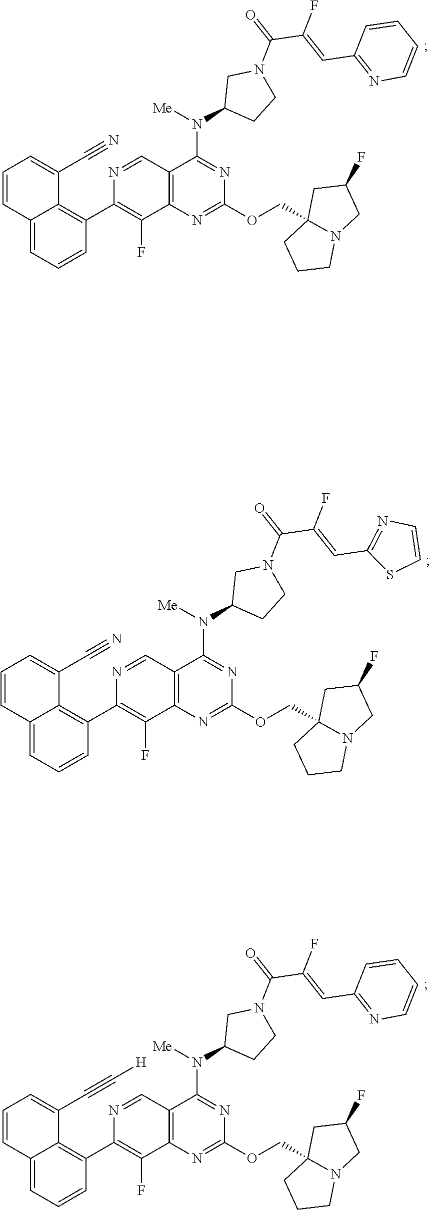 [SMILES: CN(c1nc(OC[C@@]23CCCN2C[C@H](F)C3)nc2c(F)c(-c3cccc4cccc(C#N)c34)ncc12)[C@@H]1CCN(C(=O)/C(F)=C/c2ccccn2)C1 ; CN(c1nc(OC[C@@]23CCCN2C[C@H](F)C3)nc2c(F)c(-c3cccc4cccc(C#N)c34)ncc12)[C@@H]1CCN(C(=O)/C(F)=C/c2nccs2)C1 ; C#Cc1cccc2cccc(-c3ncc4c(N(C)[C@@H]5CCN(C(=O)/C(F)=C/c6ccccn6)C5)nc(OC[C@@]56CCCN5C[C@H](F)C6)nc4c3F)c12]

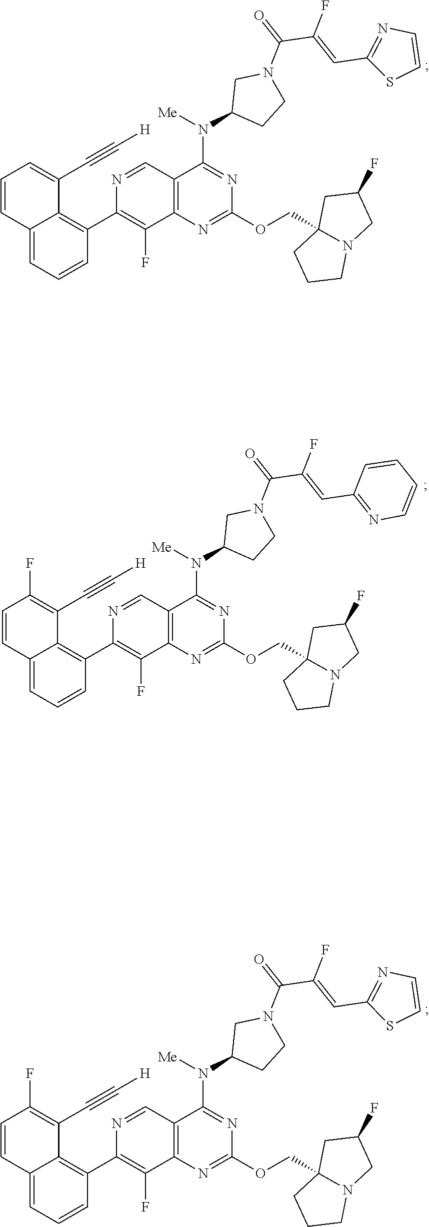 [SMILES: C#Cc1cccc2cccc(-c3ncc4c(N(C)[C@@H]5CCN(C(=O)/C(F)=C/c6nccs6)C5)nc(OC[C@@]56CCCN5C[C@H](F)C6)nc4c3F)c12 ; C#Cc1c(F)ccc2cccc(-c3ncc4c(N(C)[C@@H]5CCN(C(=O)/C(F)=C/c6ccccn6)C5)nc(OC[C@@]56CCCN5C[C@H](F)C6)nc4c3F)c12 ; C#Cc1c(F)ccc2cccc(-c3ncc4c(N(C)[C@@H]5CCN(C(=O)/C(F)=C/c6nccs6)C5)nc(OC[C@@]56CCCN5C[C@H](F)C6)nc4c3F)c12]

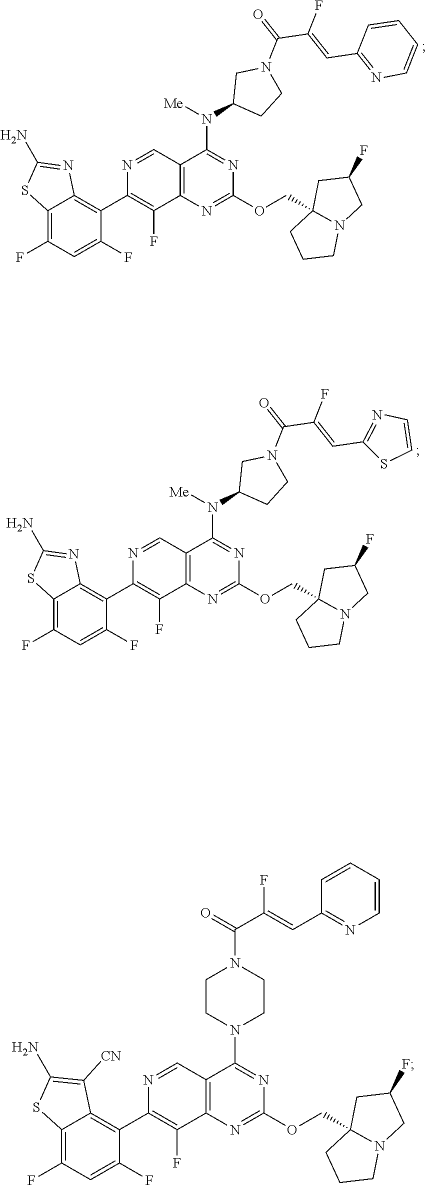 [SMILES: CN(c1nc(OC[C@@]23CCCN2C[C@H](F)C3)nc2c(F)c(-c3c(F)cc(F)c4sc(N)nc34)ncc12)[C@@H]1CCN(C(=O)/C(F)=C/c2ccccn2)C1 ; CN(c1nc(OC[C@@]23CCCN2C[C@H](F)C3)nc2c(F)c(-c3c(F)cc(F)c4sc(N)nc34)ncc12)[C@@H]1CCN(C(=O)/C(F)=C/c2nccs2)C1 ; N#Cc1c(N)sc2c(F)cc(F)c(-c3ncc4c(N5CCN(C(=O)/C(F)=C/c6ccccn6)CC5)nc(OC[C@@]56CCCN5C[C@H](F)C6)nc4c3F)c12]

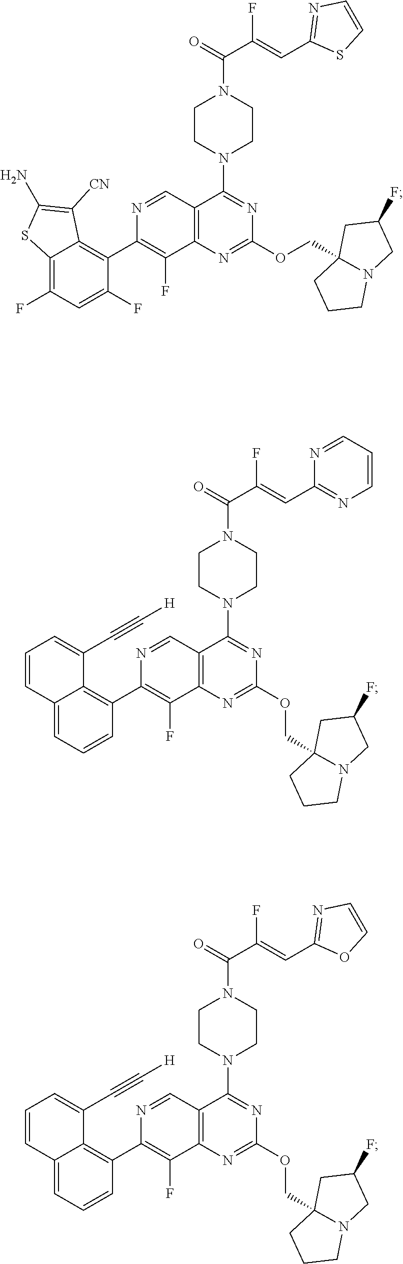 [SMILES: N#Cc1c(N)sc2c(F)cc(F)c(-c3ncc4c(N5CCN(C(=O)/C(F)=C/c6nccs6)CC5)nc(OC[C@@]56CCCN5C[C@H](F)C6)nc4c3F)c12 ; C#Cc1cccc2cccc(-c3ncc4c(N5CCN(C(=O)/C(F)=C/c6ncccn6)CC5)nc(OC[C@@]56CCCN5C[C@H](F)C6)nc4c3F)c12 ; C#Cc1cccc2cccc(-c3ncc4c(N5CCN(C(=O)/C(F)=C/c6ncco6)CC5)nc(OC[C@@]56CCCN5C[C@H](F)C6)nc4c3F)c12]

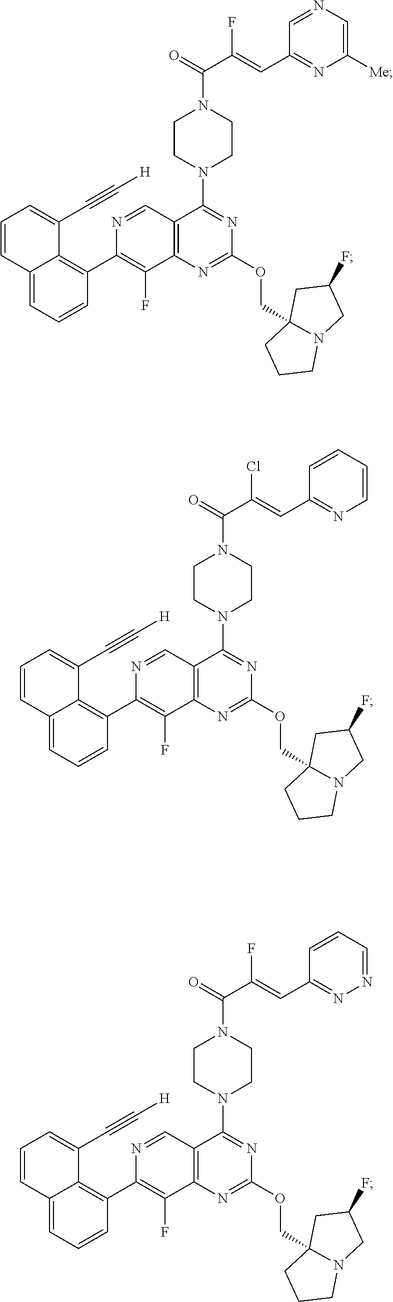 [SMILES: C#Cc1cccc2cccc(-c3ncc4c(N5CCN(C(=O)/C(F)=C/c6cncc(C)n6)CC5)nc(OC[C@@]56CCCN5C[C@H](F)C6)nc4c3F)c12 ; C#Cc1cccc2cccc(-c3ncc4c(N5CCN(C(=O)/C(Cl)=C/c6ccccn6)CC5)nc(OC[C@@]56CCCN5C[C@H](F)C6)nc4c3F)c12 ; C#Cc1cccc2cccc(-c3ncc4c(N5CCN(C(=O)/C(F)=C/c6cccnn6)CC5)nc(OC[C@@]56CCCN5C[C@H](F)C6)nc4c3F)c12]

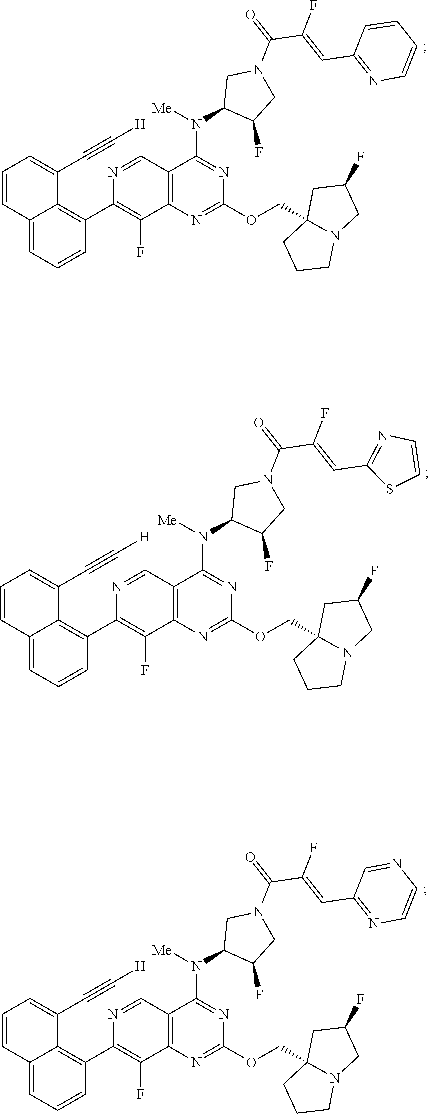 [SMILES: C#Cc1cccc2cccc(-c3ncc4c(N(C)[C@H]5CN(C(=O)/C(F)=C/c6ccccn6)C[C@H]5F)nc(OC[C@@]56CCCN5C[C@H](F)C6)nc4c3F)c12 ; C#Cc1cccc2cccc(-c3ncc4c(N(C)[C@H]5CN(C(=O)/C(F)=C/c6nccs6)C[C@H]5F)nc(OC[C@@]56CCCN5C[C@H](F)C6)nc4c3F)c12 ; C#Cc1cccc2cccc(-c3ncc4c(N(C)[C@H]5CN(C(=O)/C(F)=C/c6cnccn6)C[C@H]5F)nc(OC[C@@]56CCCN5C[C@H](F)C6)nc4c3F)c12]

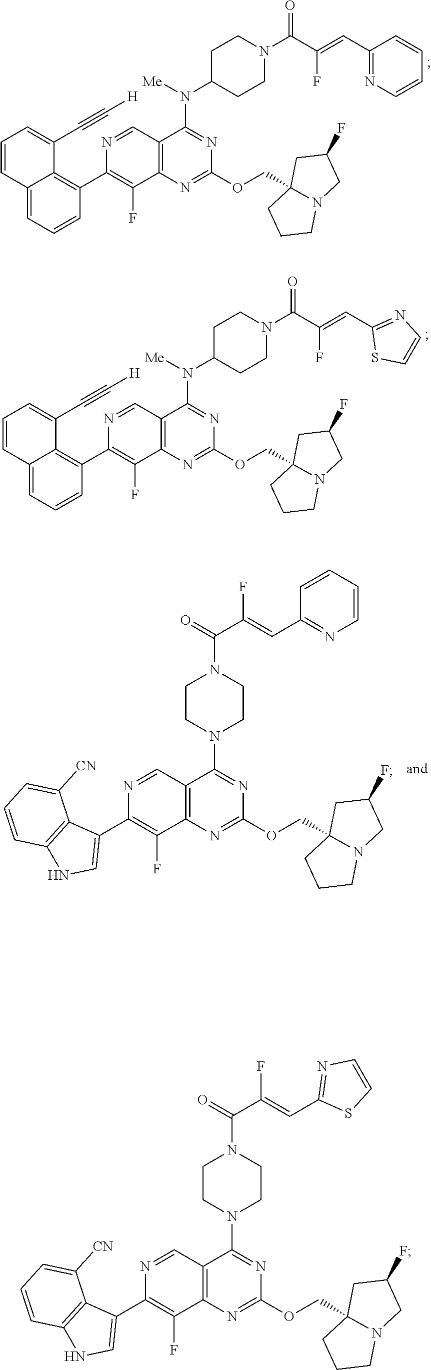 [SMILES: C#Cc1cccc2cccc(-c3ncc4c(N(C)C5CCN(C(=O)/C(F)=C/c6ccccn6)CC5)nc(OC[C@@]56CCCN5C[C@H](F)C6)nc4c3F)c12 ; C#Cc1cccc2cccc(-c3ncc4c(N(C)C5CCN(C(=O)/C(F)=C/c6nccs6)CC5)nc(OC[C@@]56CCCN5C[C@H](F)C6)nc4c3F)c12 ; N#Cc1cccc2[nH]cc(-c3ncc4c(N5CCN(C(=O)/C(F)=C/c6ccccn6)CC5)nc(OC[C@@]56CCCN5C[C@H](F)C6)nc4c3F)c12 ; N#Cc1cccc2[nH]cc(-c3ncc4c(N5CCN(C(=O)/C(F)=C/c6nccs6)CC5)nc(OC[C@@]56CCCN5C[C@H](F)C6)nc4c3F)c12]

or a pharmaceutically acceptable salt thereof.

In some aspects, the present disclosure provides a compound selected from:
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(thiazol-2-yl)prop-2-en-1-one;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(pyridin-2-yl)prop-2-en-1-one;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(4-methylpyridin-2-yl)prop-2-en-1-one;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(oxazol-2-yl)prop-2-en-1-one;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(1-methyl-1H-imidazol-2-yl)prop-2-en-1-one;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(5-methylpyridin-2-yl)prop-2-en-1-one;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-chloro-3-(pyrimidin-yl)prop-2-en-1-one;
(Z)-3-(5-bromopyridin-2-yl)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoroprop-2-en-1-one;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(6-methylpyridin-2-yl)prop-2-en-1-one;
(Z)-3-(4-bromothiazol-2-yl)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoroprop-2-en-1-one;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(pyridazin-3-yl)prop-2-en-1-one;
(Z)—N-((1R, 4R)-2-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-2-azabicyclo[2.2.1]heptan-4-yl)-2-fluoro-3-(pyridin-2-yl)acrylamide;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(5-methyl-1,3,4-thiadiazol-2-yl)prop-2-en-1-one;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(6-(methoxymethyl)pyridin-2-yl)prop-2-en-1-one;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(pyrazin-2-yl)prop-2-en-1-one;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(6-methoxypyridin-2-yl)prop-2-en-1-one;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(pyrimidin-4-yl)prop-2-en-1-one;
2-((S)-4-(7-(8-chloronaphthalen-1-yl)-8-fluoro-2-(((S)-1-methylpyrrolidin-2-yl)methoxy)pyrido[4,3-d]pyrimidin-4-yl)-1-((Z)-2-fluoro-3-(pyridin-2-yl)acryloyl)piperazin-2-yl)acetonitrile;
2-((S)-4-(8-fluoro-2-(((S)-1-methylpyrrolidin-2-yl)methoxy)-7-(naphthalen-1-yl)pyrido[4,3-d]pyrimidin-4-yl)-1-((Z)-2-fluoro-3-(pyridin-2-yl)acryloyl)piperazin-2-yl)acetonitrile;
2-((S)-4-(7-(8-chloronaphthalen-1-yl)-8-fluoro-2-(((S)-1-methylpyrrolidin-2-yl)methoxy)pyrido[4,3-d]pyrimidin-4-yl)-1-((Z)-2-fluoro-3-(pyrimidin-2-yl)acryloyl)piperazin-2-yl)acetonitrile;
2-((S)-4-(7-(8-chloronaphthalen-1-yl)-8-fluoro-2-(((S)-1-methylpyrrolidin-2-yl)methoxy)pyrido[4,3-d]pyrimidin-4-yl)-1-((Z)-2-fluoro-3-(thiazol-2-yl)acryloyl)piperazin-2-yl)acetonitrile;
2-((S)-4-(8-fluoro-2-(((S)-1-methylpyrrolidin-2-yl)methoxy)-7-(naphthalen-1-yl)pyrido[4,3-d]pyrimidin-4-yl)-1-((Z)-2-fluoro-3-(pyridin-2-yl)acryloyl)piperazin-2-yl)acetonitrile;
(S,Z)-1-(4-(7-(8-chloronaphthalen-1-yl)-8-fluoro-2-((1-methylpyrrolidin-2-yl)methoxy)pyrido[4,3-d]pyrimidin-4-yl)piperazin-1-yl)-2-fluoro-3-(thiazol-2-yl)prop-2-en-1-one;
2-((S)-1-((Z)-2-fluoro-3-(thiazol-2-yl)acryloyl)-4-(8-fluoro-7-(2-fluoro-5-hydroxyphenyl)-2-(((S)-1-methylpyrrolidin-2-yl)methoxy)pyrido[4,3-d]pyrimidin-4-yl)piperazin-2-yl)acetonitrile;
2-((S)-4-(7-(8-ethylnaphthalen-1-yl)-8-fluoro-2-(((S)-1-methylpyrrolidin-2-yl)methoxy)pyrido[4,3-d]pyrimidin-4-yl)-1-((Z)-2-fluoro-3-(thiazol-2-yl)acryloyl)piperazin-2-yl)acetonitrile;
(S,Z)-1-(4-(7-(8-ethynylnaphthalen-1-yl)-8-fluoro-2-((1-methylpyrrolidin-2-yl)methoxy)pyrido[4,3-d]pyrimidin-4-yl)piperazin-1-yl)-2-fluoro-3-(pyridin-2-yl)prop-2-en-1-one;
(Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(6-methylpyrazin-2-yl)prop-2-en-1-one;
(Z)-1-(4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridin-2-yl)prop-2-en-1-one;
(Z)-1-(4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(thiazol-2-yl)prop-2-en-1-one;
(Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridin-2-yl)prop-2-en-1-one;
(Z)-2-fluoro-1-(4-(8-fluoro-2-(((2R, 7aS)-2-fluorotetrahydro-1H-pyrrolizin-7a(5H)-yl)methoxy)-7-(5-methyl-1H-indazol-4-yl)pyrido[4,3-d]pyrimidin-4-yl)piperazin-1-yl)-3-(pyridin-2-yl)prop-2-en-1-one;
(Z)-1-(4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridin-2-yl)prop-2-en-1-one;
2-((S)-4-(8-fluoro-2-(((2R, 7aS)-2-fluorotetrahydro-1H-pyrrolizin-7a(5H)-yl)methoxy)-7-(2-(trifluoromethyl)phenyl)pyrido[4,3-d]pyrimidin-4-yl)-1-((Z)-2-fluoro-3-(thiazol-2-yl)acryloyl)piperazin-2-yl)acetonitrile;
2-((S)-4-(8-fluoro-2-(((2R, 7aS)-2-fluorotetrahydro-1H-pyrrolizin-7a(5H)-yl)methoxy)-7-(2-(trifluoromethyl)phenyl)pyrido[4,3-d]pyrimidin-4-yl)-1-((Z)-2-fluoro-3-(pyridin-2-yl)acryloyl)piperazin-2-yl)acetonitrile;
2-((S)-4-(7-(benzo[b]thiophen-3-yl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(thiazol-2-yl)acryloyl)piperazin-2-yl)acetonitrile;
2-((S)-4-(7-(benzo[b]thiophen-3-yl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(pyridin-2-yl)acryloyl)piperazin-2-yl)acetonitrile;
2-((S)-1-((Z)-2-fluoro-3-(pyridin-2-yl)acryloyl)-4-(8-fluoro-7-(7-fluoronaphthalen-1-yl)-2-(((S)-1-methylpyrrolidin-2-yl)methoxy)pyrido[4,3-d]pyrimidin-4-yl)piperazin-2-yl)acetonitrile;
2-((S)-1-((Z)-2-fluoro-3-(thiazol-2-yl)acryloyl)-4-(8-fluoro-7-(7-fluoronaphthalen-1-yl)-2-(((S)-1-methylpyrrolidin-2-yl)methoxy)pyrido[4,3-d]pyrimidin-4-yl)piperazin-2-yl)acetonitrile;
(Z)-2-fluoro-1-(4-(8-fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)piperazin-1-yl)-3-(pyridin-2-yl)prop-2-en-1-one;
(Z)-2-fluoro-1-(4-(8-fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)piperazin-1-yl)-3-(thiazol-2-yl)prop-2-en-1-one;
2-((S)-4-(7-(2,5-difluorophenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(pyridin-2-yl)acryloyl)piperazin-2-yl)acetonitrile;
2-((S)-4-(7-(2,5-difluorophenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(thiazol-2-yl)acryloyl)piperazin-2-yl)acetonitrile;
2-((S)-4-(7-(2-(difluoromethyl)-5-fluorophenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(pyridin-2-yl)acryloyl)piperazin-2-yl)acetonitrile;
2-((S)-4-(7-(2-(difluoromethyl)-5-fluorophenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(thiazol-2-yl)acryloyl)piperazin-2-yl)acetonitrile;
2-((S)-1-((Z)-2-fluoro-3-(pyridin-2-yl)acryloyl)-4-(8-fluoro-7-(5-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile;
2-((S)-1-((Z)-2-fluoro-3-(thiazol-2-yl)acryloyl)-4-(8-fluoro-7-(5-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile;
2-((S)-1-((Z)-2-fluoro-3-(thiazol-2-yl)acryloyl)-4-(8-fluoro-7-(5-chloro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile;
(Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(thiazol-2-yl)prop-2-en-1-one;
(Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridazin-2-yl)prop-2-en-1-one;
8-(8-fluoro-4-(4-((Z)-2-fluoro-3-(pyridin-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7-yl)-1-naphthonitrile;
8-(8-fluoro-4-(4-((Z)-2-fluoro-3-(thiazol-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7-yl)-1-naphthonitrile;
2-((S)-4-(8-fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)-1-((Z)-2-fluoro-3-(pyridin-2-yl)acryloyl)piperazin-2-yl)acetonitrile;
2-((S)-4-(8-fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)-1-((Z)-2-fluoro-3-(thiazol-2-yl)acryloyl)piperazin-2-yl)acetonitrile;
(Z)-1-(4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(thiazol-2-yl)prop-2-en-1-one;
(Z)-1-(4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(oxazol-2-yl)prop-2-en-1-one;
(Z)-1-(4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridazin-3-yl)prop-2-en-1-one;
(Z)-1-(4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyrazin-2-yl)prop-2-en-1-one;
(Z)-1-(4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyrimidin-2-yl)prop-2-en-1-one;
(Z)-1-(4-(7-(8-ethynyl-7-fluoro-3-hydroxynaphthalen-1-yl)-8-fluoro-2-(((4aS, 7aR)-1-methyloctahydro-4aH-cyclopenta[b]pyridin-4a-yl)methoxy)pyrido[4,3-d]pyrimidin-4-yl)piperazin-1-yl)-2-fluoro-3-(thiazol-2-yl)prop-2-en-1-one;
(Z)-1-(4-(7-(8-ethynyl-7-fluoro-3-hydroxynaphthalen-1-yl)-8-fluoro-2-(((4aS, 7aR)-1-methyloctahydro-4aH-cyclopenta[b]pyridin-4a-yl)methoxy)pyrido[4,3-d]pyrimidin-4-yl)piperazin-1-yl)-2-fluoro-3-(pyridin-2-yl)prop-2-en-1-one;
2-((S)-1-((Z)-2-fluoro-3-(pyridin-2-yl)acryloyl)-4-(8-fluoro-7-(5-fluoro-2,3-dihydro-4H-benzo[b][1,4]oxazin-4-yl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile;
2-((S)-1-((Z)-2-fluoro-3-(thiazol-2-yl)acryloyl)-4-(8-fluoro-7-(5-fluoro-2,3-dihydro-4H-benzo[b][1,4]oxazin-4-yl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile;
(Z)-1-((S)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(thiazol-2-yl)prop-2-en-1-one;
(Z)-1-((S)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyridin-2-yl)prop-2-en-1-one;
(Z)-1-((S)-4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(thiazol-2-yl)prop-2-en-1-one;
(Z)-1-((S)-4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyridin-2-yl)prop-2-en-1-one;
(Z)-1-((S)-4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyrimidin-2-yl)prop-2-en-1-one;
(Z)-1-(4-(7-(2-amino-5,7-difluorobenzo[d]thiazol-4-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(thiazol-2-yl)prop-2-en-1-one;
(Z)-1-(4-(7-(2-amino-7-fluorobenzo[d]thiazol-4-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(thiazol-2-yl)prop-2-en-1-one;
(Z)-1-((S)-4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(thiazol-2-yl)prop-2-en-1-one;
(Z)-1-((S)-4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(thiazol-2-yl)prop-2-en-1-one; 2-((S)-1-((Z)-2-fluoro-3-(thiazol-2-yl)acryloyl)-4-(8-fluoro-7-(3-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile; 2-((S)-4-(8-fluoro-2-(((2R, 7aS)-2-fluorotetrahydro-1H-pyrrolizin-7a(5H)-yl)methoxy)-7-(3-hydroxyphenyl)pyrido[4,3-d]pyrimidin-4-yl)-1-((Z)-2-fluoro-3-(thiazol-2-yl)acryloyl)piperazin-2-yl)acetonitrile;
(Z)-1-(3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidin-1-yl)-2-fluoro-3-(thiazol-2-yl)prop-2-en-1-one trans-isomer 1;
(Z)-1-(3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidin-1-yl)-2-fluoro-3-(thiazol-2-yl)prop-2-en-1-one trans-isomer 2;
(Z)-1-(3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidin-1-yl)-2-fluoro-3-(thiazol-2-yl)prop-2-en-1-one cis-isomer;
(Z)-1-(3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidin-1-yl)-2-fluoro-3-(thiazol-2-yl)prop-2-en-1-one cis-isomer 2;
(Z)-1-(3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidin-1-yl)-2-fluoro-3-(pyridin-2-yl)prop-2-en-1-one cis-isomer 1;
(Z)-1-(3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidin-1-yl)-2-fluoro-3-(pyridin-2-yl)prop-2-en-1-one cis-isomer 2;
(Z)-1-(3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidin-1-yl)-2-fluoro-3-(pyridin-2-yl)prop-2-en-1-one trans-isomer 1;
(Z)-1-(3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidin-1-yl)-2-fluoro-3-(pyridin-2-yl)prop-2-en-1-one trans-isomer 2;
(Z)-1-(3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidin-1-yl)-2-fluoro-3-(pyridin-2-yl)prop-2-en-1-one;
(Z)-1-((3R)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyridin-2-yl)prop-2-en-1-one;
(Z)-1-((3R)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(thiazol-2-yl)prop-2-en-1-one;
(Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-1,4-diazepan-1-yl)-2-fluoro-3-(pyridin-2-yl)prop-2-en-1-one;
(Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-1,4-diazepan-1-yl)-2-fluoro-3-(thiazol-2-yl)prop-2-en-1-one;
2-((2S)-4-(7-(2-cyclopropylphenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(pyridin-2-yl)acryloyl)piperazin-2-yl)acetonitrile;
2-((2S)-4-(7-(2-cyclopropylphenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(thiazol-2-yl)acryloyl)piperazin-2-yl)acetonitrile;
2-((2S)-4-(7-(2-cyclobutylphenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(pyridin-2-yl)acryloyl)piperazin-2-yl)acetonitrile;
2-((2S)-4-(7-(2-cyclobutylphenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(thiazol-2-yl)acryloyl)piperazin-2-yl)acetonitrile;
(Z)-1-((2S, 5R)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-2,5-dimethylpiperazin-1-yl)-2-fluoro-3-(pyridin-2-yl)prop-2-en-1-one;
(Z)-1-((3R)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyrimidin-2-yl)prop-2-en-1-one;
(Z)-1-((3aR, 6aR)-1-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)hexahydropyrrolo[3,4-b]pyrrol-5(1H)-yl)-2-fluoro-3-(pyridin-2-yl)prop-2-en-1-one;
(Z)-1-((3aR, 6aR)-1-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)hexahydropyrrolo[3,4-b]pyrrol-5(1H)-yl)-2-fluoro-3-(pyrimidin-2-yl)prop-2-en-1-one;
(Z)-1-(4-(7-(8-ethynyl-4-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridin-2-yl)prop-2-en-1-one;
(Z)-1-(4-(7-(8-ethynyl-4-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyrimidin-2-yl)prop-2-en-1-one;
(Z)-1-((S)-4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyridin-2-yl)prop-2-en-1-one;
(Z)-1-((S)-4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyrimidin-2-yl)prop-2-en-1-one;
(Z)-1-(2-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-2,7-diazaspiro[3.5]nonan-7-yl)-2-fluoro-3-(pyridin-2-yl)prop-2-en-1-one;
(Z)-1-(2-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-2,7-diazaspiro[3.5]nonan-7-yl)-2-fluoro-3-(pyrimidin-2-yl)prop-2-en-1-one;
(Z)-1-(7-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-2,7-diazaspiro[3.5]nonan-2-yl)-2-fluoro-3-(pyridin-2-yl)prop-2-en-1-one;
2-((S)-4-(8-fluoro-2-(((2R, 7aS)-2-fluorotetrahydro-1H-pyrrolizin-7a(5H)-yl)methoxy)-7-(5-hydroxy-2-(trifluoromethyl)phenyl)pyrido[4,3-d]pyrimidin-4-yl)-1-((Z)-2-fluoro-3-(pyridin-2-yl)acryloyl)piperazin-2-yl)acetonitrile;
2-((S)-4-(8-fluoro-2-(((2R, 7aS)-2-fluorotetrahydro-1H-pyrrolizin-7a(5H)-yl)methoxy)-7-(5-hydroxy-2-(trifluoromethyl)phenyl)pyrido[4,3-d]pyrimidin-4-yl)-1-((Z)-2-fluoro-3-(thiazol-2-yl)acryloyl)piperazin-2-yl)acetonitrile;
(Z)-1-(4-(7-(8-ethynyl-5-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridin-2-yl)prop-2-en-1-one;
(Z)-1-(4-(7-(8-ethynyl-5-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyrimidin-2-yl)prop-2-en-1-one;
(Z)-1-(4-(7-(8-ethynyl-6,7-difluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyrimidin-2-yl)prop-2-en-1-one;
(Z)-1-(4-(7-(8-ethynyl-6,7-difluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridin-2-yl)prop-2-en-1-one;
(Z)-1-((3aR, 6aS)-5-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)hexahydropyrrolo[3,4-c]pyrrol-2(1H)-yl)-2-fluoro-3-(pyridin-2-yl)prop-2-en-1-one;
(Z)-1-((3aR, 6aS)-5-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)hexahydropyrrolo[3,4-c]pyrrol-2(1H)-yl)-2-fluoro-3-(pyrimidin-2-yl)prop-2-en-1-one;
(Z)-1-(6-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-1,6-diazaspiro[3.4]octan-1-yl)-2-fluoro-3-(pyridin-2-yl)prop-2-en-1-one
(Z)-1-(6-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-1,6-diazaspiro[3.4]octan-1-yl)-2-fluoro-3-(thiazol-2-yl)prop-2-en-1-one;
(Z)-1-(6-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-1,6-diazaspiro[3.4]octan-1-yl)-2-fluoro-3-(pyridin-2-yl)prop-2-en-1-one;
(Z)-1-(6-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-1,6-diazaspiro[3.4]octan-1-yl)-2-fluoro-3-(pyrimidin-2-yl)prop-2-en-1-one; 2-((S)-4-(8-fluoro-2-(((2R, 7aS)-2-fluorotetrahydro-1H-pyrrolizin-7a(5H)-yl)methoxy)-7-(5-methyl-1H-indazol-6-yl)pyrido[4,3-d]pyrimidin-4-yl)-1-((Z)-2-fluoro-3-(pyridin-2-yl)acryloyl)piperazin-2-yl)acetonitrile;
(Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)hexahydropyrrolo[3,2-b]pyrrol-1(2H)-yl)-2-fluoro-3-(pyridin-2-yl)prop-2-en-1-one;
(Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)hexahydropyrrolo[3,2-b]pyrrol-1(2H)-yl)-2-fluoro-3-(pyridin-2-yl)prop-2-en-1-one;
(Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)-5-methoxypyrido[4,3-d]pyrimidin-4-yl)piperazin-1-yl)-2-fluoro-3-(pyridin-2-yl)prop-2-en-1-one;
(Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)-5-methoxypyrido[4,3-d]pyrimidin-4-yl)piperazin-1-yl)-2-fluoro-3-(thiazol-2-yl)prop-2-en-1-one;
(S,Z)-1-(4-(2-((1-(3-azidopropyl)pyrrolidin-2-yl)methoxy)-7-(8-ethynyl-7-fluoro-3-hydroxynaphthalen-1-yl)-8-fluoropyrido[4,3-d]pyrimidin-4-yl)piperazin-1-yl)-2-fluoro-3-(pyridin-2-yl)prop-2-en-1-one;
N-((1-(3-((S)-2-(((7-(8-ethynyl-7-fluoro-3-hydroxynaphthalen-1-yl)-8-fluoro-4-(4-((Z)-2-fluoro-3-(pyridin-2-yl)acryloyl)piperazin-1-yl)pyrido[4,3-d]pyrimidin-2-yl)oxy)methyl)pyrrolidin-1-yl)propyl)-1H-1,2,3-triazol-4-yl)methyl)-5-((3aS,4S,6aR)-2-oxohexahydro-1H-thieno[3,4-d]imidazol-4-yl)pentanamide;
8-(8-fluoro-4-(((R)-1-((Z)-2-fluoro-3-(pyridin-2-yl)acryloyl)pyrrolidin-3-yl)(methyl)amino)-2-(((2R, 7aS)-2-fluorotetrahydro-1H-pyrrolizin-7a(5H)-yl)methoxy)pyrido[4,3-d]pyrimidin-7-yl)-1-naphthonitrile;
8-(8-fluoro-4-(((R)-1-((Z)-2-fluoro-3-(thiazol-2-yl)acryloyl)pyrrolidin-3-yl)(methyl)amino)-2-(((2R, 7aS)-2-fluorotetrahydro-1H-pyrrolizin-7a(5H)-yl)methoxy)pyrido[4,3-d]pyrimidin-7-yl)-1-naphthonitrile;
(Z)-1-((R)-3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoro-3-(pyridin-2-yl)prop-2-en-1-one;
(Z)-1-((R)-3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoro-3-(thiazol-2-yl)prop-2-en-1-one;
(Z)-1-((R)-3-((7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoro-3-(pyridin-2-yl)prop-2-en-1-one;
(Z)-1-((R)-3-((7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoro-3-(thiazol-2-yl)prop-2-en-1-one;
(Z)-1-((3R)-3-((7-(2-amino-5,7-difluorobenzo[d]thiazol-4-yl)-8-fluoro-2-(((7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoro-3-(pyridin-2-yl)prop-2-en-1-one; tert-butyl(5,7-difluoro-4-(8-fluoro-4-(((R)-1-((Z)-2-fluoro-3-(thiazol-2-yl)acryloyl)pyrrolidin-3-yl)(methyl)amino)-2-(((7aS)-2-fluorotetrahydro-1H-pyrrolizin-7a(5H)-yl)methoxy)pyrido[4,3-d]pyrimidin-7-yl)benzo[d]thiazol-2-yl)carbamate;
2-amino-7-fluoro-4-(8-fluoro-4-(4-((Z)-2-fluoro-3-(pyridin-2-yl)acryloyl)piperazin-1-yl)-2-(((7aS)-2-fluorotetrahydro-1H-pyrrolizin-7a(5H)-yl)methoxy)pyrido[4,3-d]pyrimidin-7-yl)benzo[b]thiophene-3-carbonitrile;
2-amino-7-fluoro-4-(8-fluoro-4-(4-((Z)-2-fluoro-3-(thiazol-2-yl)acryloyl)piperazin-1-yl)-2-(((7aS)-2-fluorotetrahydro-1H-pyrrolizin-7a(5H)-yl)methoxy)pyrido[4,3-d]pyrimidin-7-yl)benzo[b]thiophene-3-carbonitrile;
(Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyrimidin-2-yl)prop-2-en-1-one;
(Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(oxazol-2-yl)prop-2-en-1-one;
(Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(6-methylpyrazin-2-yl)prop-2-en-1-one;
(Z)-2-chloro-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-3-(pyridin-2-yl)prop-2-en-1-one;
(Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridazin-3-yl)prop-2-en-1-one;
(Z)-1-((3S,4R)-3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-4-fluoropyrrolidin-1-yl)-2-fluoro-3-(pyridin-2-yl)prop-2-en-1-one;
(Z)-1-((3S,4R)-3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-4-fluoropyrrolidin-1-yl)-2-fluoro-3-(thiazol-2-yl)prop-2-en-1-one;
(Z)-1-((3S,4R)-3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-4-fluoropyrrolidin-1-yl)-2-fluoro-3-(pyrazin-2-yl)prop-2-en-1-one;
(Z)-1-(4-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)piperidin-1-yl)-2-fluoro-3-(pyridin-2-yl)prop-2-en-1-one;
(Z)-1-(4-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)piperidin-1-yl)-2-fluoro-3-(thiazol-2-yl)prop-2-en-1-one;
3-(8-fluoro-4-(4-((Z)-2-fluoro-3-(pyridin-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7-yl)-1H-indole-4-carbonitrile; and
3-(8-fluoro-4-(4-((Z)-2-fluoro-3-(thiazol-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7-yl)-1H-indole-4-carbonitrile;
   or a pharmaceutically acceptable salt thereof.

In some aspects, the present disclosure provides an atropisomer of a compound of any of the prior aspects. In certain aspects, the compound is a stable atropisomer as described herein.

In some aspects, the present disclosure provides a pharmaceutical composition comprising a compound of any of the prior aspects, or a pharmaceutically acceptable salt thereof, and one or more pharmaceutically acceptable excipients.

In some aspects, the present disclosure provides an oral dosage form comprising a compound of any of the prior aspects, or a pharmaceutically acceptable salt thereof, and one or more pharmaceutically acceptable excipients.

In some aspects, the present disclosure provides a method of treating cancer expressing KRAS G12C, G12D and/or G12V mutation in a subject in need thereof, the method comprising administering to the subject a compound of any of the prior aspects, or a pharmaceutically acceptable salt thereof.

In some aspects, the present disclosure provides a method of treating cancer expressing KRAS G12C mutation in a subject in need thereof, the method comprising administering to the subject a compound of any of the prior aspects, or a pharmaceutically acceptable salt thereof.

In some aspects, the present disclosure provides a method for treating a cancer susceptible to KRAS G12C inhibition in a subject in need thereof, the method comprising administering to the subject a compound of any of the prior aspects, or a pharmaceutically acceptable salt thereof.

In some aspects, the present disclosure provides a method for treating a cancer in a subject in need thereof, the method comprising administering to the subject a compound of any of the prior aspects, or a pharmaceutically acceptable salt thereof, wherein the cancer is lung cancer, colorectal cancer, pancreatic cancer, breast cancer, bladder cancer, cervical cancer, ovarian cancer, gastric cancer or cancer of the uterus.

In some aspects, the present disclosure provides a method for treating a cancer in a subject in need thereof, the method comprising administering to the subject a compound of any of the prior aspects, or a pharmaceutically acceptable salt thereof, wherein the cancer is non-small cell lung cancer.

In some aspects of the method, the compound is an atropisomer of a compound of any of the prior aspects. In certain aspects, the compound is a stable atropisomer as described herein.

In another aspect, the present disclosure provides a method for inhibiting KRAS G12C activity in a in a cell, comprising contacting the cell with a therapeutically effective amount of a compound of formula (I), or a pharmaceutically acceptable salt thereof, or a pharmaceutical composition thereof as defined herein. In one aspect, the contacting is in vitro. In one aspect, the contacting is in vivo.

In some aspects, the present disclosure provides a method of inhibiting cell proliferation, in vitro or in vivo, the method comprising contacting a cell with a therapeutically effective amount of a compound of formula (I), or a pharmaceutically acceptable salt thereof, or a pharmaceutical composition thereof as defined herein.

In another aspect, the present disclosure provides a compound of formula (I), or a pharmaceutically acceptable salt thereof, for use in the inhibition of KRAS G12C.

In another aspect, the present disclosure provides a compound of formula (I), or a pharmaceutically acceptable salt thereof, or a pharmaceutical composition thereof as defined herein, for use in the treatment of a KRAS G12C-associated disease or disorder.

In another aspect, the present disclosure provides a use of a compound of formula (I), or a pharmaceutically acceptable salt thereof, as defined herein in the manufacture of a medicament for the treatment of cancer. In some aspects, the cancer is lung cancer. In some aspects, the cancer is non-small cell lung cancer.

In another aspect, the present disclosure provides the use of a compound of formula (I), or a pharmaceutically acceptable salt thereof, as defined herein in the manufacture of a medicament for the inhibition of activity of KRAS G12C.

In another aspect, the present disclosure provides the use of a compound of formula (I), or a pharmaceutically acceptable salt thereof, as defined herein, in the manufacture of a medicament for the treatment of a KRAS G12C-associated disease or disorder.

### DETAILED DESCRIPTION

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the phrase “or a pharmaceutically acceptable salt thereof” refers to at least one compound, or at least one salt of the compound, or a combination thereof. For example, “a compound of Formula (I) or a pharmaceutically acceptable salt thereof” includes, but is not limited to, a compound of Formula (I), two compounds of Formula (I), a pharmaceutically acceptable salt of a compound of Formula (I), a compound of Formula (I) and one or more pharmaceutically acceptable salts of the compound of Formula (I), and two or more pharmaceutically acceptable salts of a compound of Formula (I).

The term “C₂-C₄alkenyl”, as used herein, refers to a group derived from a straight or branched chain hydrocarbon containing from two to four carbon atoms and one double bond.

The term “C₁-C₃alkoxy”, as used herein, refers to a C₁-C₃alkyl group attached to the parent molecular moiety through an oxygen atom.

The term “C₁-C₃alkoxy C₁-C₆alkyl,” as used herein, refers to a C₁-C₃alkoxy group attached to the parent molecular moiety through a C₁-C₆alkyl group.

The term “C₁-C₃alkoxycarbonyl”, as used herein, refers to a C₁-C₃alkoxy group attached to the parent molecular moiety through a carbonyl group.

The term “C₁-C₃alkyl”, as used herein, refers to a group derived from a straight or branched chain saturated hydrocarbon containing from one to three carbon atoms.

The term “C₁-C₃alkylcarbonyl”, as used herein, refers to a C₁-C₃alkyl group attached to the parent molecular moiety through a carbonyl group.

The term “C₂-C₄alkynyl”, as used herein, refers to a group derived from a straight or branched chain hydrocarbon containing from two to four carbon atoms and one triple bond.

The term “amino,” as used herein, refers to —NH₂.

The term “amino C₁-C₃alkyl,” as used herein, refers to an amino group attached to the parent molecular moiety through a C₁-C₃alkyl group.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl.

The term “aryl C₁-C₆alkyl,” as used herein refers to an aryl group attached to the parent molecular moiety through a C₁-C₆alkyl group.

The term “azido,” as used herein, refers to —N₃.

The term “azido C₁-C₆alkyl,” as used herein refers to an azido group attached to the parent molecular moiety through a C₁-C₆alkyl group.

The term “carboxy,” as used herein, refers to —CO₂ H.

The term “carboxy C₁-C₆alkyl,” as used herein, refers to a C₁-C₆alkyl group substituted with one, two, or three carboxy groups.

The term “C(O)—C₁-C₆alkylheteroaryl,” as used herein, refers to an heteroaryl C₁-C₆alkyl group attached to the parent molecular moiety through a carbonyl group.

The term “cyano”, as used herein, refers to —CN.

The term “C₃-C₄cycloalkyl”, as used herein, refers to a saturated monocyclic hydrocarbon ring system having three or four carbon atoms and zero heteroatoms.

The term “C₃-C₆cycloalkyl”, as used herein, refers to a saturated monocyclic hydrocarbon ring system having three to six carbon atoms and zero heteroatoms.

The term “C₃-C₆cycloalkyl C₁-C₆alkyl”, as used herein, refers to a C₃-C₆cycloalkyl attached to the parent molecular moiety through a C₁-C₆alkyl group.

The term “di(C₁-C₆alkyl)amino”, as used herein, refers to —NR^{z}R^{z′}, wherein R^{z} and R^{z′} are the same or different C₁-C₆alkyl groups.

The term “di(C₁-C₃alkyl)amino C₂-C₆alkyl,” as used herein, refers to —(C₂-C₆alkyl)NR^{z}R^{z′}, wherein R^{z} and R^{z′} are the same or different C₁-C₆alkyl groups.

The terms “halo” and “halogen”, as used herein, refer to F, Cl, Br, or I.

The term “halo C₁-C₃alkoxy”, as used herein, refers to a C₁-C₃alkoxy group substituted with one, two, or three halogen atoms.

The term “halo C₁-C₃alkyl”, as used herein, refers to a C₁-C₃alkyl group substituted with one, two, or three halogen atoms.

The term “halo C₁-C₆alkyl”, as used herein, refers to a C₁-C₆alkyl group substituted with one to six halogen atoms.

The term “heteroaryl,” as used herein, refers to an aromatic five- or six-membered ring where at least one atom is selected from N, O, and S, and the remaining atoms are carbon. The term “heteroaryl” also includes bicyclic systems where a heteroaryl ring is fused to a four- to six-membered aromatic or non-aromatic ring containing zero, one, or two additional heteroatoms selected from N, O, and S; and tricyclic systems where a bicyclic system is fused to a four- to six-membered aromatic or non-aromatic ring containing zero, one, or two additional heteroatoms selected from N, O, and S. The heteroaryl groups are attached to the parent molecular moiety through any substitutable carbon or nitrogen atom in the group. Representative examples of heteroaryl groups include, but are not limited to, alloxazine, benzo[1,2-d:4,5-d′]bisthiazole, benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, purine, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl.

In those aspects where the term “heteroaryl,” includes bicyclic systems, an aromatic five- or six-membered ring having at least one atom selected from N, O, and S is fused to a four- to six-membered aromatic or non-aromatic ring containing zero, one, or two additional heteroatoms selected from N, O, and S. In those aspects where the term “heteroaryl,” includes tricyclic systems, a bicyclic system defined in the prior sentence is fused to a four- to six-membered aromatic or non-aromatic ring containing zero, one, or two additional heteroatoms selected from N, O, and S.

The term “heteroaryl C₁-C₃alkyl, as used herein, refers to a heteroaryl group attached to the parent molecular moiety through a C₁-C₃alkyl group.

The term “heteroaryl C₁-C₆alkyl, as used herein, refers to a heteroaryl group attached to the parent molecular moiety through a C₁-C₆alkyl group.

The term “heterocyclyl”, as used herein, refers to a five-, six-, seven-, eight-, nine-, ten-, eleven-, or twelve-membered saturated or partially unsaturated ring containing one, two, or three heteroatoms independently selected from nitrogen, oxygen, and sulfur. The term “heterocyclyl” also includes groups in which the heterocyclyl ring is fused to one, two, or three four- to six-membered aromatic or non-aromatic carbocyclic rings or monocyclic heterocyclyl groups. The term “heterocyclyl” also includes monocyclic or polycyclic heterocyclyl group as described above which are further substituted with one or more spirocyclic groups that are attached to the heterocyclyl group through a spiro carbon. Examples of heterocyclyl groups include, but are not limited to, dihydro-1′H,3′H,5′H-dispiro[cyclopropane-1,2′-pyrrolizine-6′,1″-cyclopropane], hexahydro-2H-1,4-dioxa-2al-azacyclopenta[cd]pentalenyl, hexahydropyrrolizinyl, indolinyl, morpholinyl, octahydroindolizinyl, octahydroquinolizinyl, piperazinyl, piperidinyl, pyrrolidinyl, tetrahydrofuranyl, tetrahydropyranyl, and thiomorpholinyl.

The term “heterocyclyl C₁-C₆alkyl, as used herein, refers to a heterocyclyl group attached to the parent molecular moiety through a C₁-C₆alkyl group.

The term “hydroxy,” as used herein, refers to —OH.

The term “hydroxyC₁-C₃alkyl,” as used herein, refers to a hydroxy group attached to the parent molecular moiety through a C₁-C₃alkyl group.

The term “hydroxyC₁-C₆alkyl,” as used herein, refers to a hydroxy group attached to the parent molecular moiety through a C₁-C₆alkyl group.

The term “NR^{a}R^{b}—C(O),” as used herein, refers to an NR^{a}R^{b} group attached to the parent molecular moiety through a carbonyl group.

The term “NR^{a}R^{b}—C(O)—C₁-C₆alkyl,” as used herein, refers to an NR^{a}R^{b}—C(O)— group attached to the parent molecular moiety through a C₁-C₆alkyl group.

The term “NR^{a}R^{b}C₁-C₆alkyl, as used herein, refers to an NR^{a}R^{b} group attached to the parent molecular moiety through a C₁-C₆alkyl group.

The term “NR^{x}R^{y}C₁-C₃alkyl, as used herein, refers to an NR^{X}R^{Y} group attached to the parent molecular moiety through a C₁-C₃alkyl group.

The term “nitro,” as used herein, refers to —NO₂.

The term “oxo,” as used herein, refers to ═O.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a compound described herein can be prepared using a radioactive isotope and the resulting radiolabeled compound can be used to develop a binding assay or for metabolism studies. Alternatively, and for the same purpose, a compound described herein can be converted to a radiolabeled form by catalytic tritiation using methods known to those skilled in the art.

Certain compounds of the present disclosure exist as stereoisomers. It should be understood that when stereochemistry is not specified, the present disclosure encompasses all stereochemical isomeric forms, or mixtures thereof, which possess the ability inhibit KRAS G12C. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure exist as atropisomers. The term “atropisomers” refers to conformational stereoisomers which occur when rotation about a single bond in the molecule is prevented, or greatly slowed, as a result of steric interactions with other parts of the molecule and the substituents at both ends of the single bond are asymmetrical (i.e., optical activity arises without requiring an asymmetric carbon center or stereocenter). Where the rotational barrier about the single bond is high enough, and interconversion between conformations is slow enough, separation and isolation of the isomeric species may be permitted. Atropisomers are enantiomers (or epimers) without a single asymmetric atom.

The atropisomers can be considered stable if the barrier to interconversion is high enough to permit the atropisomers to undergo little or no interconversion at room temperature for at least a week. In some aspects the atropisomers undergo little or no interconversion at room temperature for at least a year. In some aspects, an atropisomeric compound of the disclosure does not undergo more than about 5% interconversion to its opposite atropisomer at room temperature during one week when the atropisomeric compound is in substantially pure form, which is generally a solid state. In some aspects, an atropisomeric compound of the disclosure does not undergo more than about 5% interconversion to its opposite atropisomer at room temperature (approximately 25° C.) during one year. In some aspects, the atropisomeric compounds of the disclosure are stable enough to undergo no more than about 5% interconversion in an aqueous pharmaceutical formulation held at 0° C. for at least one week. The present chemical entities, pharmaceutical compositions and methods are meant to include all such possible atropisomers, including racemic mixtures, diastereomeric mixtures, epimeric mixtures, optically pure forms of single atropisomers, and intermediate mixtures.

The energy barrier to thermal racemization of atropisomers may be determined by the steric hindrance to free rotation of one or more bonds forming a chiral axis. Certain biaryl compounds exhibit atropisomerism where rotation around an interannular bond lacking C₂ symmetry is restricted. The free energy barrier for isomerization (enantiomerization) is a measure of the stability of the interannular bond with respect to rotation. Optical and thermal excitation can promote racemization of such isomers, dependent on electronic and steric factors.

Ortho-substituted biaryl compounds may exhibit this type of conformational, rotational isomerism. Such biaryls are enantiomeric, chiral atropisomers where the sp²-sp² carbon-carbon, interannular bond between the aryl rings has a sufficiently high energy barrier to prevent free rotation, and where substituents W¹≠W² and W³≠W⁴ render the molecule asymmetric.

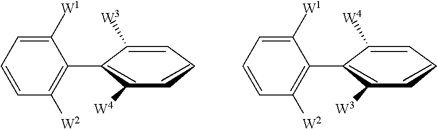

The steric interaction between W¹:W³, W¹:W⁴, and/or W²:W⁴, W²:W³ is large enough to make the planar conformation an energy maximum. Two non-planar, axially chiral enantiomers then exist as atropisomers when their interconversion is slow enough such that they can be isolated free of each other. Bold lines and dashed lines in the figures shown above indicate those moieties, or portions of the molecule, which are sterically restricted due to a rotational energy barrier. Balded moieties exist orthogonally above the plane of the page, and dashed moieties exist orthogonally below the plane of the page. The ‘flat’ part of the molecule (the left ring in each of the two depicted biaryls) is in the plane of the page.

The pharmaceutical compositions of the disclosure can include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci., 66:1-19 (1977)). The salts can be obtained during the final isolation and purification of the compounds described herein, or separately be reacting a free base function of the compound with a suitable acid or by reacting an acidic group of the compound with a suitable base. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

### Pharmaceutical Compositions

In another aspect, the present disclosure provides a composition, e.g., a pharmaceutical composition, containing one or a combination of the compounds described within the present disclosure, formulated together with a pharmaceutically acceptable carrier. Pharmaceutical compositions of the disclosure also can be administered in combination therapy, i.e., combined with other agents, as described herein.

As used herein, “pharmaceutically acceptable carrier” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound can be coated in a material to protect the compound from the action of acids and other natural conditions that can inactivate the compound.

The pharmaceutical compositions of the present disclosure can be administered via one or more routes of administration using one or more of a variety of methods known in the art. As will be appreciated by the skilled artisan, the route and/or mode of administration will vary depending upon the desired results. In some aspects, the routes of administration for compounds of the disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are reduced pressure drying and freeze-drying (lyophilization) that yield a powder of the active ingredient plus any additional desired ingredient from a previously sterile-filtered solution thereof.

Examples of suitable aqueous and non-aqueous carriers that can be employed in the pharmaceutical compositions of the disclosure include water, ethanol, polyols (such as glycerol, propylene glycol, polyethylene glycol, and the like), and suitable mixtures thereof, vegetable oils, and injectable organic esters. Proper fluidity can be maintained, for example, by the use of coating materials, such as lecithin, by the maintenance of the required particle size in the case of dispersions, and by the use of surfactants.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution or as a liquid with ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, the compounds of the disclosure can be administered via a non-parenteral route, such as a topical, epidermal or mucosal route of administration, for example, intranasally, orally, vaginally, rectally, sublingually or topically.

Any pharmaceutical composition contemplated herein can, for example, be delivered orally via any acceptable and suitable oral preparation. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one non-toxic pharmaceutically acceptable excipient suitable for the manufacture of tablets.

An aqueous suspension can be prepared, for example, by admixing at least one compound of Formula (I) and/or at least one pharmaceutically acceptable salt thereof with at least one excipient suitable for the manufacture of an aqueous suspension, including, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example, heptadecathylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) and/or at least one pharmaceutically acceptable salt thereof in either a vegetable oil, such as, for example, arachis oil, sesame oil, and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described herein above, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent, at least one suspending agent, and/or at least one preservative. Suitable dispersing agents, wetting agents, and suspending agents are already described above. Exemplary preservatives include, but are not limited to, for example, anti-oxidants, e.g., ascorbic acid. In addition, dispersible powders and granules can also contain at least one excipient, including, but not limited to, for example, sweetening agents, flavoring agents, and coloring agents.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Robinson, J. R., ed., Sustained and Controlled Release Drug Delivery Systems, Marcel Dekker, Inc., New York (1978).

Therapeutic compositions can be administered with medical devices known in the art. For example, in one aspect, a therapeutic composition of the disclosure can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medication through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain aspects, the compounds of the present disclosure can be administered parenterally, i.e., by injection, including, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and/or infusion.

In some aspects, the compounds of the present disclosure can be administered orally, i.e., via a gelatin capsule, tablet, hard or soft capsule, or a liquid capsule.

### Use of KRAS Inhibitors/Methods of Treating

Administration of a therapeutic agent described herein may include administration of a therapeutically effective amount of therapeutic agent. The term “therapeutically effective amount” as used herein refers, without limitation, to an amount of a therapeutic agent to treat a condition treatable by administration of a composition comprising the KRAS inhibitors described herein. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration.

For administration of the compounds described herein, the dosage ranges from about 0.0001 to 100 mg/kg, and more usually 0.01 to 40 mg/kg, of the host body weight. An exemplary treatment regime entails administration once per day, bi-weekly, tri-weekly, weekly, once every two weeks, once every three weeks, once every four weeks, once a month, once every 3 months or once every three to 6 months.

The disclosed compounds strongly inhibit anchorage-independent cell growth and therefore have the potential to inhibit tumor metastasis. Accordingly, in another aspect the disclosure provides a method for inhibiting tumor metastasis, the method comprising administering an effective amount a pharmaceutical composition of comprising any of the compounds disclosed herein and a pharmaceutically acceptable carrier to a subject in need thereof.

Ras mutations including but not limited to KRAS mutations have also been identified in hematological malignancies (e.g., cancers that affect blood, bone marrow and/or lymph nodes). Accordingly, certain aspects are directed to administration of a disclosed compounds (e.g., in the form of a pharmaceutical composition) to a patient in need of treatment of a hematological malignancy. Such malignancies include, but are not limited to, leukemias and lymphomas. For example, the presently disclosed compounds can be used for treatment of diseases such as Acute lymphoblastic leukemia (ALL), Acute myelogenous leukemia (AML), Chronic lymphocytic leukemia (CLL), small lymphocytic lymphoma (SLL), Chronic myelogenous leukemia (CML), Acute monocytic leukemia (AMoL) and/or other leukemias. In other aspects, the compounds are useful for treatment of lymphomas such as all subtypes of Hodgkins lymphoma or non-Hodgkins lymphoma.

Determining whether a tumor or cancer comprises a KRAS mutation can be undertaken by assessing the nucleotide sequence encoding the KRAS protein, by assessing the amino acid sequence of KRAS protein, or by assessing the characteristics of a putative KRAS mutant protein. The sequence of wild-type human KRAS proteins is known in the art.

Methods for detecting a KRAS mutation are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some aspects, samples are evaluated for KRAS mutations including by real-time PCR. In real-time PCR, fluorescent probes specific for the KRAS mutation are used. When a mutation is present, the probe binds and fluorescence is detected. In some aspects, the KRAS mutation is identified using a direct sequencing method of specific regions (e.g., exon 2 and/or exon 3) in the KRAS gene, for example. This technique will identify all possible mutations in the region sequenced.

Methods for detecting a mutation in a KRAS protein are known by those of skill in the art. These methods include, but are not limited to, detection of a KRAS mutant using a binding agent (e.g., an antibody) specific for the mutant protein, protein electrophoresis and Western blotting, and direct peptide sequencing.

Methods for determining whether a tumor or cancer comprises a KRAS mutation can use a variety of samples. In some aspects, the sample is taken from a subject having a tumor or cancer. In some aspects, the sample is taken from a subject having a cancer or tumor. In some aspects, the sample is a fresh tumor/cancer sample. In some aspects, the sample is a frozen tumor/cancer sample. In some aspects, the sample is a formalin-fixed paraffin-embedded sample. In some aspects, the sample is processed to a cell lysate. In some aspects, the sample is processed to DNA or RNA. The disclosure also relates to a method of treating a hyperproliferative disorder in a mammal that comprises administering to said mammal a therapeutically effective amount of a compound of the present disclosure, or a pharmaceutically acceptable salt, ester, prodrug, solvate, hydrate or derivative thereof. In some aspects, said method relates to the treatment of cancer such as acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, isletcell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some aspects, said method relates to the treatment of a non-cancerous hyperproliferative disorder such as benign hyperplasia of the skin (e.g., psoriasis), restenosis, or prostate (e.g., benign prostatic hypertrophy (BPH)).

In certain aspects, the disclosure relates to methods for treatment of lung cancers, the methods comprise administering an effective amount of any of the above-described compound (or a pharmaceutical composition comprising the same) to a subject in need thereof. In certain aspects the lung cancer is a non-small cell lung carcinoma (NSCLC), for example adenocarcinoma, squamous-cell lung carcinoma or large-cell lung carcinoma. In other aspects, the lung cancer is a small cell lung carcinoma. Other lung cancers treatable with the disclosed compounds include, but are not limited to, glandular tumors, carcinoid tumors and undifferentiated carcinomas. Subjects that can be treated with compounds of the disclosure, or pharmaceutically acceptable salt, ester, prodrug, solvate, tautomer, hydrate or derivative of said compounds, according to the methods of this disclosure include, for example, subjects that have been diagnosed as having acute myeloid leukemia, acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germcell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer,lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasalcavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some aspects subjects that are treated with the compounds of the disclosure include subjects that have been diagnosed as having a non-cancerous hyperproliferative disorder such as benign hyperplasia of the skin (e.g., psoriasis), restenosis, or prostate (e.g., benign pro static hypertrophy (BPH)). The disclosure further provides methods of modulating a mutant KRAS protein activity by contacting the protein with an effective amount of a compound of the disclosure. Modulation can be inhibiting or activating protein activity. In some aspects, the disclosure provides methods of inhibiting protein activity by contacting the mutant KRAS protein with an effective amount of a compound of the disclosure in solution. In some aspects, the disclosure provides methods of inhibiting the mutant KRAS protein activity by contacting a cell, tissue, organ that express the protein of interest. In some aspects, the disclosure provides methods of inhibiting protein activity in a subject including but not limited to rodents and mammal (e.g., human) by administering into the subject an effective amount of a compound of the disclosure. In some aspects, the percentage modulation exceeds 25%, 30%, 40%, 50%, 60%, 70%, 80%, or 90%. In some aspects, the percentage of inhibiting exceeds 25%, 30%, 40%, 50%, 60%, 70%, 80%, or 90%. In some aspects, the disclosure provides methods of inhibiting KRAS activity in a cell by contacting said cell with an amount of a compound of the disclosure sufficient to inhibit the activity of a KRAS mutant in said cell. In some aspects, the disclosure provides methods of inhibiting mutant KRAS in a tissue by contacting said tissue with an amount of a compound of the disclosure sufficient to inhibit the activity of mutant KRAS in said tissue. In some aspects, the disclosure provides methods of inhibiting KRAS in an organism by contacting said organism with an amount of a compound of the disclosure sufficient to inhibit the activity of KRAS in said organism. In some aspects, the disclosure provides methods of inhibiting KRAS activity in an animal by contacting said animal with an amount of a compound of the disclosure sufficient to inhibit the activity of KRAS in said animal. In some aspects, the disclosure provides methods of inhibiting KRAS including in a mammal by contacting said mammal with an amount of a compound of the disclosure sufficient to inhibit the activity of KRAS in said mammal. In some aspects, the disclosure provides methods of inhibiting KRAS activity in a human by contacting said human with an amount of a compond of the disclosure sufficient to inhibit the activity of KRAS in said human. The present disclosure provides methods of treating a disease mediated by KRAS activity in a subject in need of such treatment. The present disclosure also provides methods for combination therapies in which an agent known to modulate other pathways, or other components of the same pathway, or even overlapping sets of target enzymes are used in combination with a compound of the present disclosure, or a pharmaceutically acceptable salt, ester, prodrug, solvate, tautomer, hydrate or derivative thereof. In one aspect, such therapy includes but is not limited to the combination of one or more compounds of the disclosure with chemotherapeutic agents, therapeutic antibodies, and radiation treatment.

Many chemotherapeutics are presently known in the art and can be used in combination with the compounds of the disclosure. In some aspects, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

The compounds described herein can be used in combination with the agents disclosed herein or other suitable agents, depending on the condition being treated. Hence, in some aspects, the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some aspects of the separate administration protocol, a compound of the disclosure and any of the agents described above are administered a few minutes apart, or a few hours apart, or a few days apart.

The compounds can be made by methods known in the art including those described below and including variations within the skill of the art. Some reagents and intermediates are known in the art. Other reagents and intermediates can be made by methods known in the art using readily available materials. Any variables (e.g., numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

### Synthesis

The aspects described herein are further defined in the following Examples. It should be understood that the Examples are given by way of illustration only. From the above discussion and the Examples, one skilled in the art can ascertain the essential characteristics of the aspects described herein, and without departing from the spirit and scope thereof, can make various changes and modifications to them to adapt to various uses and conditions. As a result, the aspects described herein are not limited by the illustrative examples set forth herein below, but rather are defined by the claims appended hereto.

### Abbreviations

The following abbreviations are used in the example section below and elsewhere herein: AA for ammonium acetate; DMF for N,N-dimethylformamide; DMSO for dimethylsulfoxide; Et for ethyl; EtOAc for ethyl acetate; h for hours; MeCN or CAN for acetonitrile MeOH for methanol; TFA for trifluoroacetic acid; and THF for tetrahyrofuran.

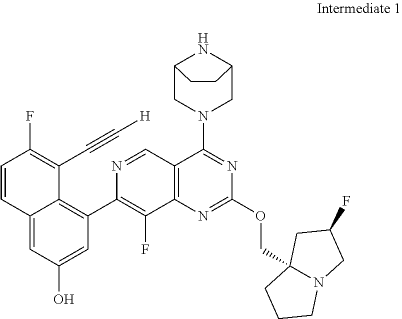 [SMILES: C#Cc1c(F)ccc2cc(O)cc(-c3ncc4c(N5CC6CCC(C5)N6)nc(OC[C@@]56CCCN5C[C@H](F)C6)nc4c3F)c12]

### (4-(4-((1R, 5S)-3,8-diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol)

This compound was prepared according to the synthetic procedure described in: WO 2021/041671.

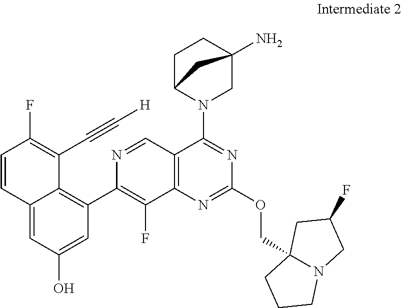 [SMILES: C#Cc1c(F)ccc2cc(O)cc(-c3ncc4c(N5C[C@@]6(N)CC[C@@H]5C6)nc(OC[C@@]56CCCN5C[C@H](F)C6)nc4c3F)c12]

### 4-(4-((1R, 4R)-4-amino-2-azabicyclo[2.2.1]heptan-2-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol

This compound was prepared by a procedure analogous to the one described for Intermediate 1, substituting in the appropriate starting material.

### Preparation of Heteroaryl Intermediates

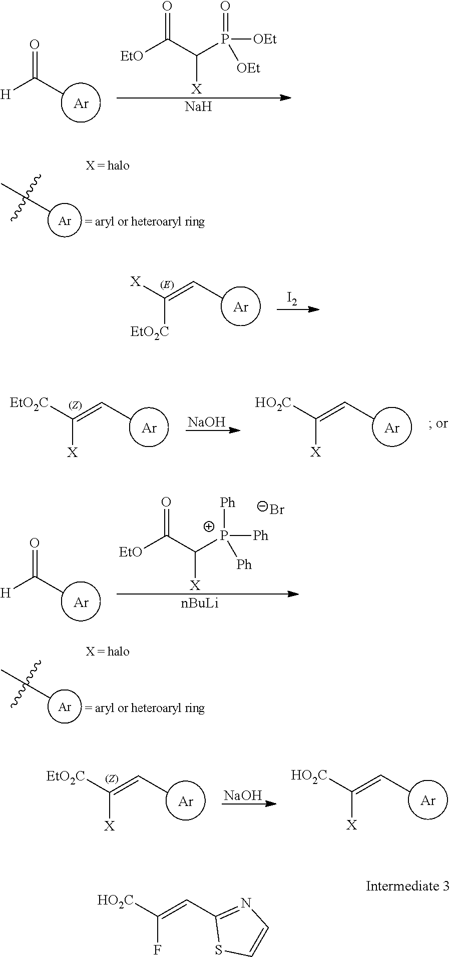

### (Z)-2-fluoro-3-(thiazol-2-yl)acrylic acid

### Step 1: Preparation of ethyl (E)-2-fluoro-3-(thiazol-2-yl)acrylate

Ethyl 2-(diethoxyphosphoryl)-2-fluoroacetate (1.1 g, 4.5 mmol) was dissolved in THE (25 mL). The solution was cooled to 0° C. Sodium hydride (60% dispersion in mineral oil, 0.18 g, 4.5 mmol) was added portionwise as a solid. The reaction mixture was stirred for 10 min and thiazole-2-carbaldehyde (0.51 g, 4.5 mmol) was added. The reaction mixture was allowed to warm to room temperature and was stirred for 1 h. The mixture was quenched by addition of saturated aqueous ammonium chloride solution (20 mL). The solution was diluted with water (20 mL) and EtOAc (150 mL). The layers were separated and the aqueous phase was further extracted with EtOAc (2×50 mL). The combined organic layers were dried over magnesium sulfate, filtered, and concentrated. The crude residue was purified by column chromatogrpahy (0→60% acetone/hexanes) to provide ethyl (E)-2-fluoro-3-(thiazol-2-yl)acrylate (10:1 E/Z as judged by ¹H NMR, 620 mg, 3.1 mmol, 68% yield). LC/MS (ESI) m/z: [M+H]⁺ calcd for CsH9FNO₂ S 202.0; found 202.2; ¹H NMR (500 MHz, CDCl₃) δ 7.94 (d, J=3.2 Hz, 1H), 7.52 (d, J=3.2 Hz, 1H), 7.34 (d, J=22.2 Hz, 1H), 4.43 (q, J=7.2 Hz, 2H), 1.41 (t, J=7.2 Hz, 3H).

### Step 2: Preparation of ethyl (Z)-2-fluoro-3-(thiazol-2-yl)acrylate

Ethyl (E)-2-fluoro-3-(thiazol-2-yl)acrylate (620 mg, 3.1 mmol) was dissolved in toluene (15 mL) and iodine (39 mg, 0.15 mmol) was added. The reaction mixture was heated at 100° C. for 7 days. The solution was concentrated and purified by column chromatography (0→100% EtOAc/hexanes) to provide ethyl (Z)-2-fluoro-3-(thiazol-2-yl)acrylate (509 mg, 2.5 mmol, 82% yield). LC/MS (ESI) m/z: [M+H]⁺ calcd for CsH9FNO₂ S 202.0; found 202.0; ¹H NMR (500 MHz, CDCl₃) δ 7.96 (dd, J=3.2, 2.6 Hz, 1H), 7.57 (d, J=3.2 Hz, 1H), 7.43 (dd, J=33.3, 0.8 Hz, 1H), 4.38 (q, J=7.2 Hz, 2H), 1.39 (t, J=7.1 Hz, 3H).

### Step 3: Preparation of (Z)-2-fluoro-3-(thiazol-2-yl)acrylic acid

Ethyl (Z)-2-fluoro-3-(thiazol-2-yl)acrylate (510 mg, 2.5 mmol) was dissolved in MeOH (15 mL). The solution was cooled to 0° C. and sodium hydroxide solution (1.0 M, 2.5 mL, 2.5 mmol) was added. The reaction mixture was stirred for 5 h. The solution was concentrated to remove the methanol. Additional water (1.5 mL) was added and the aqueous solution was cooled to 0° C. HCl solution (1.0 M, 2.5 mL, 2.5 mmol) was added dropwise. After 10 min, a white solid precipitated. The solid was collected by filtration and was washed with MeCN. The solid was dried under vacuum to provide (Z)-2-fluoro-3-(thiazol-2-yl)acrylic acid (337 mg, 1.9 mmol, 77% yield) as a white solid. LC/MS (ESI) m/z: [M+H]⁺ calcd for C₆H₅FNO₂ S 174.0; found 173.8; ¹H NMR (500 MHz, DMSO-d₆) δ 8.04 (s, 2H), 7.29 (d, J=34.5 Hz, 1H).

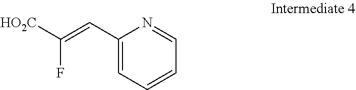 [SMILES: O=C(O)/C(F)=C/c1ccccn1]

### (Z)-2-fluoro-3-(pyridin-2-yl)acrylic acid

### Step 1: Preparation of ethyl (E)-2-fluoro-3-(pyridin-2-yl)acrylate

Ethyl 2-(diethoxyphosphoryl)-2-fluoroacetate (1.5 g, 6.2 mmol) was dissolved in THE (31 mL). The solution was cooled to 0° C. Sodium hydride (60% dispersion in mineral oil, 0.25 g, 6.2 mmol) was added portionwise as a solid. The reaction mixture was stirred for 10 min and picolinaldehyde (0.66 g, 6.2 mmol) was added. The reaction mixture was allowed to warm to room temperature and was stirred for 1 h. The mixture was quenched by addition of saturated aqueous ammonium chloride solution (15 mL). The solution was diluted with water (20 mL) and EtOAc (100 mL). The layers were separated and the aqueous phase was further extracted with EtOAc (2×50 mL). The combined organic layers were dried over magnesium sulfate, filtered, and concentrated. The crude residue was purified by column chromatogrpahy (0-100% EtOAc/hexanes) to provide ethyl (E)-2-fluoro-3-(pyridin-2-yl)acrylate (3:1 E/Z mixture as judged by ¹H NMR, 910 mg, 4.6 mmol, 75% yield). LC/MS (ESI) m/z: [M+H]⁺ calcd for C₁₀H₁₁FNO₂ 196.1; found 196.1; E isomer reported: ¹H NMR (500 MHz, CDCl₃) δ 8.59 (dd, J=4.9, 1.8 Hz, 1H), 7.67 (ddd, J=7.9, 7.6, 1.8 Hz, 1H), 7.56 (d, J=7.9 Hz, 1H), 7.21 (dd, J=7.6, 4.9 Hz, 1H), 6.90 (d, J=20.4 Hz, 1H), 4.25 (q, J=7.2 Hz, 2H), 1.23 (t, J=7.1 Hz, 3H).

### Step 2: Preparation of ethyl (Z)-2-fluoro-3-(pyridin-2-yl)acrylate

Ethyl (E)-2-fluoro-3-(pyridin-2-yl)acrylate (420 mg, 2.1 mmol) was dissolved in toluene (10 mL) and iodine (27 mg, 0.15 mmol) was added. The reaction mixture was heated at 100° C. for 7 days. The solution was concentrated and purified by column chromatography (0-100% EtOAc/hexanes) to provide ethyl (Z)-2-fluoro-3-(pyridin-2-yl)acrylate (240 mg, 1.2 mmol, 58% yield). LC/MS (ESI) m/z: [M+H]⁺ calcd for C₁₀H₁₁FNO₂ 196.1; found 196.1; ¹H NMR (500 MHz, CDCl₃) δ 8.66 (ddd, J=5.0, 1.8, 0.8 Hz, 1H), 7.88 (ddd, J=8.0, 1.2, 0.8 Hz, 1H), 7.75 (ddd, J=8.0, 7.8, 1.8 Hz, 1H), 7.25 (ddd, J=7.8, 5.0, 1.2 Hz, 1H), 7.14 (d, J=34.9 Hz, 1H), 4.36 (q, J=7.2 Hz, 2H), 1.38 (t, J=7.2 Hz, 3H).

### Step 3: Preparation of (Z)-2-fluoro-3-(pyridin-2-yl)acrylic acid

Ethyl (Z)-2-fluoro-3-(pyridin-2-yl)acrylate (415 mg, 2.1 mmol) was dissolved in MeOH (15 mL). The solution was cooled to 0° C. and sodium hydroxide solution (1.0 M, 2.1 mL, 2.1 mmol) was added. The reaction mixture was stirred for 5 h. The solution was concentrated to remove the methanol. Additional water (2.0 mL) was added and the aqueous solution was cooled to 0° C. HCl solution (1.0 M, 2.1 mL, 2.1 mmol) was added dropwise. After 10 min, a white solid precipitated. The solid was collected by filtration and was washed with Et₂O. The solid was dried under vacuum to provide (Z)-2-fluoro-3-(pyridin-2-yl)acrylic acid (235 mg, 1.4 mmol, 66% yield) as a white solid. LC/MS (ESI) m/z: [M+H]⁺ calcd for CsH7FNO₂ 168.0; found 167.8. ¹H NMR (500 MHz, DMSO-d₆) δ 8.66 (ddd, J=4.8, 1.8, 0.8 Hz, 1H), 7.89 (ddd, J=8.0, 7.5, 1.8 Hz, 1H), 7.81 (ddd, J=8.0, 1.1, 0.8 Hz, 1H), 7.40 (dd, J=7.5, 4.8, 1.1 Hz, 1H), 6.97 (d, J=35.3 Hz, 1H).

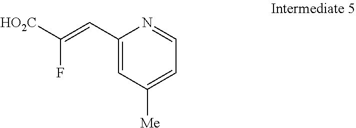 [SMILES: Cc1ccnc(/C=C(\F)C(=O)O)c1]

### (Z)-2-fluoro-3-(4-methylpyridin-2-yl)acrylic acid

### Step 1: Preparation of ethyl (Z)-2-fluoro-3-(4-methylpyridin-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and 2-formyl-4-picoline (125 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated and the aqueous phase was further extracted with EtOAc (2×20 mL). The combined organic layers were dried over magnesium sulfate, filtered, and concentrated. The crude residue was purified by reverse phase HPLC (column: Xbridge C18, 30 mm×100 mm, 5 m particles; flow rate: 42.5 mL/min; column temperature: 25° C.; gradient: 95:5 H₂O:MeCN:0.05% TFA→5:95 H₂O:MeCN:0.05% TFA; λ=220 nm) to provide ethyl (Z)-2-fluoro-3-(4-methylpyridin-2-yl)acrylate (32 mg, 0.15 mmol, 15% yield). LC/MS (ESI) m/z: [M+H]⁺ calcd for C₁₁H₁₃FNO₂ 210.2; found 209.8; ¹H NMR (500 MHz, CDCl₃) δ 8.51 (d, J=5.0 Hz, 1H), 7.70 (s, 1H), 7.11 (d, J=35.0 Hz, 1H), 7.07 (d, J=5.0 Hz, 2H), 4.35 (q, J=7.2 Hz, 2H), 2.40 (s, 3H), 1.37 (t, J=7.2 Hz, 3H).

### Step 2: Preparation of (Z)-2-fluoro-3-(4-methylpyridin-2-yl)acrylic acid

Ethyl (Z)-2-fluoro-3-(4-methylpyridin-2-yl)acrylate (32 mg, 0.15 mmol) was dissolved in MeOH (1.5 mL). The solution was cooled to 0° C. and sodium hydroxide solution (1.0 M, 0.15 mL, 0.15 mmol) was added. The reaction mixture was stirred for 2 h. The solution was concentrated to remove the methanol. Additional water (1.0 mL) was added and the aqueous solution was cooled to 0° C. HCl solution (1.0 M, 0.15 mL, 0.15 mmol) was added dropwise. The solution was frozen and directly lyophilized to provide (Z)-2-fluoro-3-(4-methylpyridin-2-yl)acrylic acid (27 mg, 0.15 mmol, quantitative yield assumed; contains 1 equiv NaCl) as a white solid. LC/MS (ESI) m/z: [M+H]⁺ calcd for C₉H₈FNO₂ 182.1; found 182.1. ¹H NMR (500 MHz, DMSO-d₆) δ 8.51 (d, J=4.9 Hz, 1H), 7.65 (s, 1H), 7.24 (d, J=4.9 Hz, 1H), 6.94 (d, J=35.3 Hz, 1H), 2.36 (s, 3H).

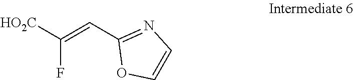 [SMILES: O=C(O)/C(F)=C/c1ncco1]

### (Z)-2-fluoro-3-(oxazol-2-yl)acrylic acid

### Step 1: Preparation of ethyl (E)-2-fluoro-3-(oxazol-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and oxazole-2-carbaldehyde (100 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated and the aqueous phase was further extracted with EtOAc (2×20 mL). The combined organic layers were dried over magnesium sulfate, filtered, and concentrated. The crude residue was purified by column chromatogrpahy (0→100% EtOAc/hexanes) to provide ethyl (E)-2-fluoro-3-(oxazol-2-yl)acrylate (6:1 E/Z as judged by 1H NMR, 165 mg, 0.89 mmol, 86% yield). LC/MS (ESI) m/z: [M+H]⁺ calcd for CsH9FNO₃ 186.0; found 186.0; E isomer reported: ¹H NMR (500 MHz, CDCl₃) δ 7.69 (d, J=0.7 Hz, 1H), 7.25 (d, J=0.7 Hz, 1H), 6.62 (d, J=17.6 Hz, 1H), 4.35 (q, J=7.2 Hz, 2H), 1.32 (t, J=7.2 Hz, 3H).

### Step 2: Preparation of ethyl (Z)-2-fluoro-3-(oxazol-2-yl)acrylate

Ethyl (E)-2-fluoro-3-(oxazol-2-yl)acrylate (165 mg, 0.89 mmol) was dissolved in toluene (2 mL) and iodine (11 mg, 0.045 mmol) was added. The reaction mixture was heated at 100° C. for 7 days. The solution was concentrated and purified by column chromatography (0→100% EtOAc/hexanes) to provide ethyl (Z)-2-fluoro-3-(oxazol-2-yl)acrylate (22 mg, 0.12 mmol, 13% yield). LC/MS (ESI) m/z: [M+H]⁺ calcd for CsH9FNO₃ 186.0; found 185.9 ¹H NMR (500 MHz, CDCl₃) δ 7.77 (s, 1H), 7.32 (s, 1H), 6.96 (d, J=31.0 Hz, 1H), 4.37 (q, J=7.2 Hz, 2H), 1.38 (t, J=7.2 Hz, 3H).

### Step 3: Preparation of (Z)-2-fluoro-3-(oxazol-2-yl)acrylic acid

Ethyl (Z)-2-fluoro-3-(oxazol-2-yl)acrylate (22 mg, 0.12 mmol) was dissolved in MeOH (1 mL). The solution was cooled to 0° C. and sodium hydroxide solution (1.0 M, 0.12 mL, 0.12 mmol) was added. The reaction mixture was stirred for 4 h. The solution was concentrated to remove the methanol. Additional water (0.3 mL) was added and the aqueous solution was cooled to 0° C. HCl solution (1.0 M, 0.12 mL, 0.12 mmol) was added dropwise. The solution was frozen and directly lyophilized to provide (Z)-2-fluoro-3-(oxazol-2-yl)acrylic acid (18 mg, 0.12 mmol, quantitative yield assumed; contains 1 equiv NaCl) as a white solid. LC/MS (ESI) m/z: [M+H]⁺ calcd for C₆H₅FNO₃ 158.0; found 157.8; ¹H NMR (600 MHz, DMSO-d₆) δ 8.30 (s, 1H), 7.47 (s, 1H), 6.90 (d, J=32.2 Hz, 1H).

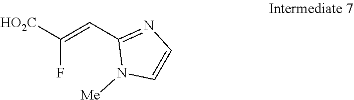 [SMILES: Cn1ccnc1/C=C(\F)C(=O)O]

### (Z)-2-fluoro-3-(I-methyl-1H-imidazol-2-yl)acrylic acid

### Step 1: Preparation of ethyl (Z)-2-fluoro-3-(I-methyl-1H-imidazol-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and 1-methylimidazole-2-carbaldehdye (114 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated and the aqueous phase was further extracted with EtOAc (2×20 mL). The combined organic layers were dried over magnesium sulfate, filtered, and concentrated. The crude residue was purified by reverse phase HPLC (column: Xbridge C18, 30 mm×100 mm, 5 μm particles; flow rate: 42.5 mL/min; column temperature: 25° C.; gradient: 95:5 H₂O:MeCN:10 mM AA→100% 5:95 H₂O:MeCN:10 nM AA; λ=220 nm) to provide ethyl (Z)-2-fluoro-3-(1-methyl-1H-imidazol-2-yl)acrylate (16 mg, 0.08 mmol, 8% yield). LC/MS (ESI) m/z: [M+H]⁺ calcd for C₉H₁₂FN₂O₂ 199.1; found 198.9; ¹H NMR (500 MHz, CDCl₃) δ 7.22 (d, J=0.7 Hz, 1H), 6.94 (d, J=0.7 Hz, 1H), 6.84 (d, J=30.5 Hz, 1H), 4.33 (q, J=7.1 Hz, 2H), 3.71 (s, 3H), 1.35 (t, J=7.1 Hz, 3H).

### Step 2: Preparation of (Z)-2-fluoro-3-(I-methyl-1H-imidazol-2-yl)acrylic acid

Ethyl (Z)-2-fluoro-3-(1-methyl-1H-imidazol-2-yl)acrylate (16 mg, 0.08 mmol) was dissolved in MeOH (1 mL). The solution was cooled to 0° C. and sodium hydroxide solution (1.0 M, 0.08 mL, 0.08 mmol) was added. The reaction mixture was stirred for 2 h. The solution was concentrated to remove the methanol. Additional water (0.3 mL) was added and the aqueous solution was cooled to 0° C. HCl solution (1.0 M, 0.08 mL, 0.08 mmol) was added dropwise. The solution was frozen and directly lyophilized to provide (Z)-2-fluoro-3-(1-methyl-1H-imidazol-2-yl)acrylic acid (13 mg, 0.08 mmol, quantitative yield assumed; contains 1 equiv NaCl) as a white solid. LC/MS (ESI) m/z: [M+H]⁺ calcd for C₇H₈FN₂O₂ 171.0; found 170.9; ¹H NMR (500 MHz, DMSO-d₆) δ 7.31 (s, 1H), 7.11 (s, 1H), 6.87 (d, J=31.6 Hz, 1H), 3.72 (s, 3H).

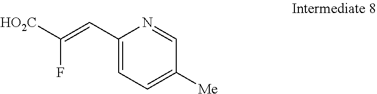 [SMILES: Cc1ccc(/C=C(\F)C(=O)O)nc1]

### (Z)-2-fluoro-3-(5-methylpyridin-2-yl)acrylic acid

### Step 1: Preparation of ethyl (Z)-2-fluoro-3-(5-methylpyridin-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and 5-methylpicolinaldehyde (125 mg, 1.0 mmol) was added. The reaction mixture was allowed to warm to room temperature and was stirred for 1 h. The mixture was quenched by addition of saturated aqueous ammonium chloride solution (15 mL). The solution was diluted with water (20 mL) and EtOAc (100 mL). The layers were separated and the aqueous phase was further extracted with EtOAc (2×50 mL). The combined organic layers were dried over magnesium sulfate, filtered, and concentrated. The crude residue was purified by reverse phase HPLC (column: Xbridge C_{18, 30} mm×100 mm, 5 m particles; flow rate: 42.5 mL/min; column temperature: 25° C.; gradient: 95:5 H₂O:MeCN:0.05% TFA→100% 5:95 H₂O:MeCN:0.05% TFA; λ=220 nm) to provide ethyl (Z)-2-fluoro-3-(4-methylpyridin-2-yl)acrylate (12 mg, 0.06 mmol, 6% yield). LC/MS (ESI) m/z: [M+H]⁺ calcd for C₁₁H₁₃FNO₂ 210.2; found 210.1¹H NMR (500 MHz, CDCL₃) δ 8.49 (d, J=1.8 Hz, 1H), 7.79 (d, J=8.2 Hz, 1H), 7.55 (dd, J=8.2, 1.8 Hz, 1H), 7.12 (d, J=35.3 Hz, 1H), 4.35 (q, J=7.2 Hz, 2H), 2.37 (s, 3H), 1.37 (t, J=7.2 Hz, 3H).

### Step 2: Preparation of (Z)-2-fluoro-3-(5-methylpyridin-2-yl)acrylic acid

Ethyl (Z)-2-fluoro-3-(5-methylpyridin-2-yl)acrylate (12 mg, 0.06 mmol) was dissolved in MeOH (0.5 mL). The solution was cooled to 0° C. and sodium hydroxide solution (1.0 M, 0.06 mL, 0.06 mmol) was added. The reaction mixture was stirred for 2 h. The solution was concentrated to remove the methanol. Additional water (1.0 mL) was added and the aqueous solution was cooled to 0° C. HCl solution (1.0 M, 0.06 mL, 0.06 mmol) was added dropwise. The solution was frozen and directly lyophilized to provide (Z)-2-fluoro-3-(5-methylpyridin-2-yl)acrylic acid (10 mg, 0.06 mmol, quantitative yield assumed; contains 1 equiv NaCl) as a white solid. LC/MS (ESI) m/z: [M+H]⁺ calcd for C₉H₈FNO₂ 182.1; found 181.6; ¹H NMR (500 MHz, DMSO-d₆) δ 8.51 (s, 1H), 7.74-7.69 (m, 2H), 6.95 (d, J=35.4 Hz, 1H), 2.33 (s, 3H).

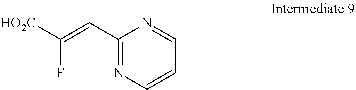 [SMILES: O=C(O)/C(F)=C/c1ncccn1]

### (Z)-2-fluoro-3-(pyrimidin-2-yl)acrylic acid

### Step 1: Preparation of ethyl (E)-2-fluoro-3-(pyrimidin-2-yl)acrylate

Ethyl 2-(diethoxyphosphoryl)-2-fluoroacetate (1.6 g, 6.6 mmol) was dissolved in THE (50 mL). The solution was cooled to 0° C. Sodium hydride (60% dispersion in mineral oil, 0.26 g, 6.6 mmol) was added portionwise as a solid. The reaction mixture was stirred for 10 min and pyrimidine-2-carbaldehyde (0.71 g, 6.6 mmol) was added. The reaction mixture was allowed to warm to room temperature and was stirred for 1 h. The mixture was quenched by addition of saturated aqueous ammonium chloride solution (20 mL). The solution was diluted with water (20 mL) and EtOAc (150 mL). The layers were separated and the aqueous phase was further extracted with EtOAc (2×50 mL). The combined organic layers were dried over magnesium sulfate, filtered, and concentrated. The crude residue was purified by column chromatogrpahy (0→90% EtOAc/hexanes) to provide ethyl (E)-2-fluoro-3-(pyrimidin-2-yl)acrylate (5:1 E/Z as judged by 1H NMR, 592 mg, 3.0 mmol, 46% yield). LC/MS (ESI) m/z: [M+H]⁺ calcd for C₉H₁₀FN₂O₂ 197.2; found 197.1; ¹H NMR (500 MHz, CDCl₃) δ 8.70 (d, J=4.9 Hz, 2H), 7.18 (t, J=4.9 Hz, 1H), 6.77 (d, J=17.5 Hz, 1H), 4.28 (q, J=7.2 Hz, 3H), 1.39 (t, J=7.2 Hz, 3H).

### Step 2: Preparation of ethyl (Z)-2-fluoro-3-(pyrimidin-2-yl)acrylate

Ethyl (E)-2-fluoro-3-(pyrimidin-2-yl)acrylate (592 mg, 3.0 mmol) was dissolved in toluene (15 mL) and iodine (38 mg, 0.15 mmol) was added. The reaction mixture was heated at 100° C. for 7 days. The solution was concentrated and purified by column chromatography (0→100% EtOAc/hexanes) to provide ethyl (Z)-2-fluoro-3-(pyrimidin-2-yl)acrylate (253 mg, 1.3 mmol, 43% yield). LC/MS (ESI) m/z: [M+H]⁺ calcd for C₉H₁₀FN₂O₂ 197.2; found 196.6; ¹H NMR (500 MHz, CDCl₃) δ 8.83 (d, J=4.9 Hz, 2H), 7.21 (t, J=4.9 Hz, 1H), 7.13 (d, J=30.8 Hz, 1H), 4.38 (q, J=7.2 Hz, 2H), 1.39 (t, J=7.2 Hz, 3H).

### Step 3: Preparation of (Z)-2-fluoro-3-(pyrimidin-2-yl)acrylic acid

Ethyl (Z)-2-fluoro-3-(pyrimidin-2-yl)acrylate (253 mg, 1.3 mmol) was dissolved in MeOH (10 mL). The solution was cooled to 0° C. and sodium hydroxide solution (1.0 M, 1.3 mL, 1.3 mmol) was added. The reaction mixture was stirred for 2 h. The solution was concentrated to remove the methanol. Additional water (1.0 mL) was added and the aqueous solution was cooled to 0° C. HCl solution (1.0 M, 2.5 mL, 2.5 mmol) was added dropwise. After 10 min, a white solid precipitated. The solid was collected by filtration and dried under vacuum to provide (Z)-2-fluoro-3-(pyrimidin-2-yl)acrylic acid (190 mg, 1.1 mmol, 88% yield) as a white solid. LC/MS (ESI) m/z: [M+H]⁺ calcd for C₇H₆FN₂O₂ 169.1; found 168.8; ¹H NMR (500 MHz, DMSO-d₆) δ 8.90 (d, J=4.9 Hz, 2H), 7.46 (t, J=4.9 Hz, 1H), 6.94 (d, J=31.5 Hz, 1H).

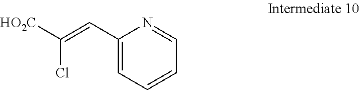 [SMILES: O=C(O)/C(Cl)=C/c1ccccn1]

### (Z)-2-chloro-3-(pyridin-2-yl)acrylic acid

### Step 1: Preparation of ethyl (Z)-2-chloro-3-(pyridine-2-yl)acrylate

Ethyl 2-chloro-2-(diethoxyphosphoryl)acetate (300 mg, 1.2 mmol) was dissolved in THE (5.8 mL). The solution was cooled to 0° C. Sodium hydride (60% dispersion in mineral oil, 46 mg, 1.2 mmol) was added portionwise as a solid. The reaction mixture was stirred for 10 min and picolinaldehyde (124 mg, 1.2 mmol) was added. The reaction mixture was allowed to warm to room temperature and was stirred for 1 h. The mixture was quenched by addition of saturated aqueous ammonium chloride solution (15 mL). The solution was diluted with water (5 mL) and EtOAc (50 mL). The layers were separated and the aqueous phase was further extracted with EtOAc (2×50 mL). The combined organic layers were dried over magnesium sulfate, filtered, and concentrated. The crude residue was purified by column chromatogrpahy (0→100% EtOAc/hexanes) to provide ethyl (Z)-2-chloro-3-(pyridin-2-yl)acrylate (79 mg, 0.37 mmol, 32% yield). LC/MS (ESI) m/z: [M+H]⁺ calcd for C₁₀H₁₁ClNO₂ 212.0; found 211.6; ¹H NMR (500 MHz, CDCl₃) δ 8.62 (ddd, J=4.8, 1.8, 1.0 Hz, 1H), 8.06 (dt, J=8.0, 1.0 Hz, 1H), 7.97 (s, 1H), 7.69 (ddd, J=8.0, 7.6, 1.8 Hz, 1H), 7.20 (ddd, J=7.6, 4.8, 1.0 Hz, 1H), 4.28 (q, J=7.1 Hz, 2H), 1.30 (t, J=7.2 Hz, 3H).

### Step 2: Preparation of (Z)-2-chloro-3-(pyridin-2-yl)acrylic acid

Ethyl (Z)-2-chloro-3-(pyridin-2-yl)acrylate (79 mg, 0.37 mmol) was dissolved in MeOH (2 mL). The solution was cooled to 0° C. and sodium hydroxide solution (1.0 M, 0.37 mL, 0.37 mmol) was added. The reaction mixture was stirred for 2 h. The solution was concentrated to remove the methanol. Additional water (1.0 mL) was added and the aqueous solution was cooled to 0° C. HCl solution (1.0 M, 0.37 mL, 0.37 mmol) was added dropwise. After 10 min, the solution was directly concentrated to provide (Z)-2-chloro-3-(pyridin-2-yl)acrylic acid (60 mg, 0.33 mmol, 88% yield) as a white solid. LC/MS (ESI) m/z: [M+H]⁺ calcd for CsH7ClNO₂ 184.0; found 183.8; ¹H NMR (500 MHz, DMSO-d₆) δ 8.72 (ddd, J=4.8, 1.8, 1.0 Hz, 1H), 8.08 (dt, J=8.0, 1.0 Hz, 1H), 7.96 (ddd, J=8.0, 7.6, 1.8 Hz, 1H), 7.93 (s, 1H), 7.46 (ddd, J=7.6, 4.8, 1.0 Hz, 1H).

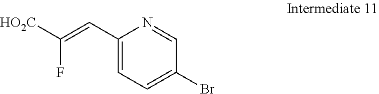 [SMILES: O=C(O)/C(F)=C/c1ccc(Br)cn1]

### (Z)-3-(5-bromopyridin-2-yl)-2-fluoroacrylic acid

### Step 1: Preparation of ethyl (Z)-2-fluoro-3-(5-bromopyridin-2-yl)acrylate

(2-ethoxy-1-fluoro-2-oxoethyl)triphenylphosphonium bromide (2.4 g, 5.4 mmol, prepared according to the synthetic procedure described in U.S. Pat. No. 9,345,791) was stirred in THE (30 mL) at 0° C. To the mixture was added dropwise nBuLi solution (1.6 M in hexanes, 3.4 mL, 5.4 mmol). The mixture was stirred for 10 minutes and then 5-bromopicolinaldehyde (1.0 g, 5.4 mmol) was added as a solid. The reaction mixture was allowed to warm to room temperature over two hours. The reaction mixture was concentrated, and the residue was directly purified by column chromatography (5→100% EtOAc/hexanes) to provide the title product as a mixture of E/Z isomers. These isomers were subsequently separated by HPLC (column: Xbridge C18, 19 mm×200 mm, 5 m particles; flow rate: 42.5 mL/min; column temperature: 25° C.; gradient: 5:95 MeCN:H₂O with 10 mM AA→95:5 MeCN:H₂O with 10 mM AA; λ=220 nm) to provide ethyl (Z)-2-fluoro-3-(5-bromopyridin-2-yl)acrylate (550 mg, 2.0 mmol, 34%). LC/MS (ESI) m/z: [M+H]⁺ calcd for C₁₀H₁₀BrFNO₂ 274.0; found 273.6; NMR: ¹H NMR (500 MHz, CDCl₃) δ 8.73 (d, J=2.3 Hz, 1H), 7.91 (dd, J=8.6, 2.3 Hz, 1H), 7.79 (d, J=8.5 Hz, 1H), 7.10 (d, J=34.3 Hz, 1H), 4.37 (q, J=7.1 Hz, 2H), 1.38 (t, J=7.2 Hz, 3H).

### Step 2: Preparation of (Z)-2-fluoro-3-(5-bromopyridin-3-yl)acrylic acid

Ethyl (Z)-2-fluoro-3-(5-bromopyridin-3-yl)acrylate (250 mg, 0.91 mmol) was dissolved in MeOH (10 mL) and sodium hydroxide solution (1.0 M, 910 μL, 0.91 mmol) was added dropwise. The mixture was stirred for 2 h. The reaction mixture was concentrated and was diluted with water (5 mL). Upon addition of an aqueous solution of hydrochloric acid (1.0 M, 910 μL, 0.91 mmol), a precipitate formed. The solid was collected by filtration and was dried under vacuum to provide (Z)-2-fluoro-3-(5-bromopyridin-3-yl)acrylic acid (217 mg, 0.88 mmol, 97%). LC/MS (ESI) m/z: [M+H]* calcd for CsH6BrFNO₂ 246.0; found 245.7; NMR: ¹H NMR (500 MHz, DMSO-d₆) δ 8.79 (d, J=2.5 Hz, 1H), 8.15 (dd, J=8.4, 2.5 Hz, 1H), 7.76 (d, J=8.4 Hz, 1H), 6.96 (d, J=34.6 Hz, 1H).

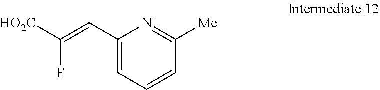 [SMILES: Cc1cccc(/C=C(\F)C(=O)O)n1]

### (Z)-2-fluoro-3-(6-methylpyridin-2-yl)acrylic acid

### Step 1: Preparation of ethyl (Z)-2-fluoro-3-(6-methylpyridin-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and 6-methylpyridine-2-carbaldehyde (125 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated and the aqueous phase was further extracted with EtOAc (2×20 mL). The combined organic layers were dried over magnesium sulfate, filtered, and concentrated. The crude residue was purified by HPLC (column: Xbridge C18, 5 m particles; gradient: 5:95 MeCN:H₂O with 0.05% TFA→95:5 MeCN:H₂O with 0.05% TFA; λ=220 nm) to provide ethyl (Z)-2-fluoro-3-(6-methylpyridin-2-yl)acrylate (31 mg, 0.15 mmol, 14% yield); LC/MS (ESI) m/z: [M+H]⁺ calcd for C₁₁H₁₃FNO₂ 210.1; found 209.7; NMR: ¹H NMR (500 MHz, CDCl₃) δ 7.71 (d, J=7.8 Hz, 1H), 7.63 (t, J=7.8 Hz, 1H), 7.10 (d, J=7.8 Hz, 1H), 7.11 (d, J=35.2 Hz, 1H), 4.34 (q, J=7.2 Hz, 2H), 2.56 (s, 3H), 1.36 (t, J=7.2 Hz, 3H).

### Step 2: Preparation of (Z)-2-fluoro-3-(6-methylpyridin-2-yl)acrylic acid

Ethyl (Z)-2-fluoro-3-(6-methylpyridin-4-yl)acrylate (31 mg, 0.15 mmol) was dissolved in MeOH (1.5 mL) and sodium hydroxide solution (1.0 M, 150 μL, 0.15 mmol) was added dropwise. The mixture was stirred for 2 h. The reaction mixture was concentrated, diluted with water (1 mL), and hydrochloric acid solution (1.0 M, 150 μL, 0.14 mmol) was added. This solution was lyophilized and the crude material was used without additional purification (quantitative yield assumed). LC/MS (ESI) m/z: [M+H]* calcd for C₉H₉FNO₂ 182.1; found 182.1; NMR: ¹H NMR (500 MHz, DMSO-d₆) δ 7.80 (t, J=7.7 Hz, 1H), 7.65 (d, J=7.7 Hz, 1H), 7.29 (d, J=7.7 Hz, 1H), 6.94 (d, J=35.4 Hz, 1H), 2.50 (s, 3H).

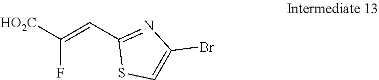 [SMILES: O=C(O)/C(F)=C/c1nc(Br)cs1]

### (Z)-3-(4-bromothiazol-2-yl)-2-fluoroacrylic acid

### Step 1: Preparation of ethyl (E)-3-(4-bromothiazol-2-yl)-2-fluoroacrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and 4-bromothiazole-2-carbaldehyde (198 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated and the aqueous phase was further extracted with EtOAc (2×20 mL). The combined organic layers were dried over magnesium sulfate, filtered, and concentrated. The crude residue was purified by column chromatogrpahy (0→100% EtOAc/hexanes) to provide ethyl (E)-3-(4-bromothiazol-2-yl)-2-fluoroacrylate (5:1 E/Z as judged by 1H NMR, 168 mg, 0.60 mmol, 60% yield). LC/MS (ESI) m/z: [M+H]⁺ calcd for C₈H₈BrFNO₂ S 279.9; found 279.9; ¹H NMR (500 MHz, CDCl₃) δ 7.41 (s, 1H), 7.28 (d, J=21.7 Hz, 2H), 4.43 (q, J=7.2 Hz, 2H), 1.41 (t, J=7.2 Hz, 3H).

### Step 2: Preparation of ethyl (Z)-3-(4-bromothiazol-2-yl)-2-fluoroacrylate

(E)-3-(4-bromothiazol-2-yl)-2-fluoroacrylate (168 mg, 0.6 mmol) was dissolved in toluene (2 mL) and iodine (15 mg, 0.06 mmol) was added. The reaction mixture was heated at 100° C. for 7 days. The solution was concentrated and purified by column chromatography (0→100% EtOAc/hexanes) to provide ethyl (Z)-3-(4-bromothiazol-2-yl)-2-fluoroacrylate (148 mg, 0.53 mmol, 88% yield). LC/MS (ESI) m/z: [M+H]⁺ calcd for C₈H₈BrFNO₂ S 279.9; found 279.9; ¹H NMR (500 MHz, CDCl₃) δ 7.44 (s, 1H), 7.38 (d, J=33.0 Hz, 1H), 4.38 (q, J=7.1 Hz, 2H), 1.38 (t, J=7.1 Hz, 3H).

### Step 3: Preparation of (Z)-3-(4-bromothiazol-2-yl)-2-fluoroacrylic acid

(Z)-3-(4-bromothiazol-2-yl)-2-fluoroacrylate (142 mg, 0.51 mmol) was dissolved in MeOH (5 mL). The solution was cooled to 0° C. and sodium hydroxide solution (1.0 M, 0.51 mL, 0.51 mmol) was added. The reaction mixture was stirred for 2 h. The solution was concentrated to remove the methanol. Additional water (1 mL) was added and the aqueous solution was cooled to 0° C. HCl solution (1.0 M, 0.51 mL, 0.51 mmol) was added dropwise. The solution was frozen and directly lyophilized to provide (Z)-3-(4-bromothiazol-2-yl)-2-fluoroacrylate as a white solid (quantitative yield assumed). LC/MS (ESI) m/z: [M+H]⁺ calcd for C₆H₄BrFNO₂ S 251.9; found 251.7; ¹H NMR (500 MHz, DMSO-d₆) δ 8.13 (s, 1H), 7.28 (d, J=33.7 Hz, 1H).

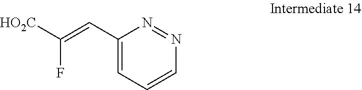 [SMILES: O=C(O)/C(F)=C/c1cccnn1]

### (Z)-2-fluoro-3-(pyridazin-3-yl)acrylic acid

### Step 1: Preparation of ethyl (E)-2-fluoro-3-(pyridazin-3-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portionwise as a solid. The reaction mixture was stirred for 10 min and pyridazine-3-carbaldehyde (110 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated and the aqueous phase was further extracted with EtOAc (2×20 mL). The combined organic layers were dried over magnesium sulfate, filtered, and concentrated. The crude residue was purified by column chromatogrpahy (0→100% EtOAc/hexanes) to provide ethyl (E)-2-fluoro-3-(pyridazin-3-yl)acrylate (3:1 E/Z as judged by 1H NMR, 168 mg, 0.60 mmol, 60% yield). LC/MS (ESI) m/z: [M+H]⁺ calcd for C₉H₁₀N₂O₂ 197.1; found 196.8; ¹H NMR (500 MHz, CDCl₃) δ 9.11 (dd, J=5.0, 1.7 Hz, 1H), 7.91 (dd, J=8.6, 1.7 Hz, 1H), 7.47 (dd, J=8.6, 5.0 Hz, 1H), 7.15 (d, J=19.9 Hz, 1H), 4.27 (q, J=7.2 Hz, 2H), 1.26 (t, J=7.2 Hz, 3H).

### Step 2: Preparation of ethyl (Z)-3-(4-bromothiazol-2-yl)-2-fluoroacrylate

Ethyl (E)-2-fluoro-3-(pyridazin-3-yl)acrylate (98 mg, 0.5 mmol) was dissolved in toluene (2 mL) and iodine (12 mg, 0.05 mmol) was added. The reaction mixture was heated at 100° C. for 7 days. The solution was concentrated and purified by column chromatography (0→100% EtOAc/hexanes) to provide ethyl (Z)-3-(4-bromothiazol-2-yl)-2-fluoroacrylate (58 mg, 0.30 mmol, 59% yield). LC/MS (ESI) m/z: [M+H]⁺ calcd for C₉H₁₀N₂O₂ 197.1; found 196.8; ¹H NMR (500 MHz, CDCl₃) δ 9.12 (dd, J=4.9, 1.6 Hz, 1H), 8.06 (dd, J=8.7, 1.6 Hz, 1H), 7.54 (dd, J=8.7, 4.9 Hz, 1H), 7.45 (d, J=34.7 Hz, 1H), 4.39 (q, J=7.2 Hz, 2H), 1.39 (t, J=7.2 Hz, 3H).

### Step 3: Preparation of (Z)-2-fluoro-3-(pyridazin-3-yl)acrylic acid

Ethyl (Z)-3-(4-bromothiazol-2-yl)-2-fluoroacrylate (55 mg, 0.28 mmol) was dissolved in MeOH (2 mL). The solution was cooled to 0° C. and sodium hydroxide solution (1.0 M, 0.28 mL, 0.28 mmol) was added. The reaction mixture was stirred for 2 h. The solution was concentrated to remove the methanol. Additional water (1 mL) was added and the aqueous solution was cooled to 0° C. HCl solution (1.0 M, 0.28 mL, 0.28 mmol) was added dropwise. The solution was frozen and directly lyophilized to provide (Z)-2-fluoro-3-(pyridazin-3-yl)acrylic acid as a white solid (quantitative yield assumed). LC/MS (ESI) m/z: [M+H]⁺ calcd for C₇H₆FN₂O₂ 169.0; found 168.8; ¹H NMR (500 MHz, DMSO-d₆) δ 9.21 (dd, J=4.9, 1.6 Hz, 1H), 8.06 (dd, J=8.6, 1.6 Hz, 1H), 7.80 (dd, J=8.6, 4.9 Hz, 1H), 7.22 (d, J=34.7 Hz, 1H).

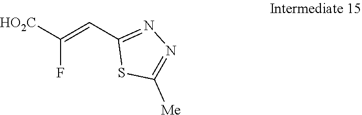 [SMILES: Cc1nnc(/C=C(\F)C(=O)O)s1]

### (Z)-2-fluoro-3-(5-methyl-1,3,4-thiadiazol-2-yl)acrylic acid

### Step 1: Preparation of ethyl (E)-2-fluoro-3-(5-methyl-1,3,4-thiadiazol-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portion wise as a solid. The reaction mixture was stirred for 10 min and 5-methyl-1,3,4-thiadiazole-2-carbaldehyde (132 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated, and the aqueous phase was further extracted with EtOAc (2×20 mL). The combined organic layers were dried over magnesium sulfate, filtered, and concentrated. The crude residue was taken on to the next step without additional purification (6:1 E/Z as judged by ¹H quantitative yield assumed). LC/MS (ESI) m/z: [M+H]⁺ calcd for C₈H₁₀FN₂O₂ S 217.0; found 216.8; E isomer reported: ¹H NMR (500 MHz, CDCL₃) δ 7.36 (d, J=21.0 Hz, 1H), 4.40 (t, J=7.2 Hz, 2H), 2.79 (s, 3H), 1.40 (t, J=7.2 Hz, 3H).

### Step 2: Preparation of ethyl (Z)-2-fluoro-3-(5-methyl-1,3,4-thiadiazol-2-yl)acrylate

Ethyl (E)-2-fluoro-3-(5-methyl-1,3,4-thiadiazol-2-yl)acrylate (223 mg assumed, 1.0 mmol) was dissolved in toluene (2 mL) and iodine (17 mg, 0.07 mmol) was added. The reaction mixture was heated at 100° C. for 7 days. The solution was concentrated and purified by column chromatography (0→100% EtOAc/hexanes) to provide ethyl (Z)-2-fluoro-3-(5-methyl-1,3,4-thiadiazol-2-yl)acrylate (63 mg, 0.29 mmol, 29% yield over two steps). LC/MS (ESI) m/z: [M+H]⁺ calcd for C₈H₁₀FN₂O₂ S 217.0; found 216.8; ¹H NMR (500 MHz, CDCl₃) δ 7.52 (d, J=32.1 Hz, 1H), 4.39 (q, J=7.2 Hz, 2H), 2.85 (s, 3H), 1.39 (t, J=7.1 Hz, 3H).

### Step 3: Preparation of (Z)-2-fluoro-3-(5-methyl-1,3,4-thiadiazol-2-yl)acrylic acid

Ethyl (Z)-2-fluoro-3-(5-methyl-1,3,4-thiadiazol-2-yl)acrylate (63 mg, 0.29 mmol) was dissolved in MeOH (2 mL). The solution was cooled to 0° C. and sodium hydroxide solution (1.0 M, 0.29 mL, 0.29 mmol) was added. The reaction mixture was stirred for 2 h. The solution was concentrated to remove the methanol. Additional water (1 mL) was added, and the aqueous solution was cooled to 0° C. HCl solution (1.0 M, 0.29 mL, 0.29 mmol) was added dropwise. A yellow precipitate formed and was collected by filtration. The solid was triturated with ether (2×2 mL) and dried under vacuum to provide (Z)-2-fluoro-3-(5-methyl-1,3,4-thiadiazol-2-yl)acrylic acid (35 mg, 0.18 mmol, 63% yield). LC/MS (ESI) m/z: [M+H]⁺ calcd for C₆H₆FN₂O₂ S 189.0; found 188.8; ¹H NMR (500 MHz, DMSO-d₆) δ 7.38 (d, J=33.3 Hz, 1H), 2.80 (s, 3H).

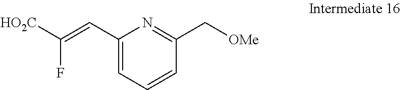 [SMILES: COCc1cccc(/C=C(\F)C(=O)O)n1]

### (Z)-2-fluoro-3-(6-(methoxymethyl)pyridin-2-yl)acrylic acid

### Step 1: Preparation of ethyl (E)-2-fluoro-3-(6-(methoxymethyl)pyridine-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portion wise as a solid. The reaction mixture was stirred for 10 min and 6-(methoxymethyl)picolinaldehyde (156 mg, 1.0 mmol) was added. The reaction mixture was allowed to warm to room temperature and was stirred for 1 h. The mixture was quenched by addition of saturated aqueous ammonium chloride solution (10 mL). The solution was diluted with water (10 mL) and EtOAc (50 mL). The layers were separated, and the aqueous phase was further extracted with EtOAc (2×20 mL). The combined organic layers were dried over magnesium sulfate, filtered, and concentrated. The crude residue was purified by column chromatography (0-100% EtOAc/hexanes) to provide ethyl (E)-2-fluoro-3-(6-(methoxymethyl)pyridin-2-yl)acrylate (3:1 E/Z as judged by 1H NMR, 120 mg, 0.50 mmol, 49% yield). LC/MS (ESI) m/z: [M+H]⁺ calcd for C₁₂H₁₅FNO₃ 240.1; found 240.0; E isomer reported: ¹H NMR (500 MHz, CDCl₃) δ 7.68 (t, J=7.8 Hz, 1H), 7.45 (d, J=7.8 Hz, 1H), 7.35 (d, J=7.8 Hz, 1H), 6.88 (d, J=20.4 Hz, 1H), 4.55 (s, 2H), 4.24 (q, J=7.2 Hz, 2H), 3.47 (s, 3H), 1.23 (t, J=7.2 Hz, 3H).

### Step 2: Preparation of ethyl (Z)-2-fluoro-3-(6-(methoxymethyl)pyridine-2-yl)acrylate

Ethyl (E)-2-fluoro-3-(6-(methoxymethyl)pyridine-2-yl)acrylate (120 mg, 0.5 mmol) was dissolved in toluene (10 mL) and iodine (6 mg, 0.025 mmol) was added. The reaction mixture was heated at 100° C. for 6 days. The solution was concentrated and purified by column chromatography (0-100% EtOAc/hexanes) to provide the desired product (59 mg, 0.25 mmol, 49% yield). LC/MS (ESI) m/z: [M+H]⁺ calcd for C₁₂H₁₅FNO₃ 240.1; found 223.8; ¹H NMR (500 MHz, CDCl₃) δ 7.79 (d, J=7.7 Hz, 1H), 7.74 (t, J=7.7 Hz, 1H), 7.37 (d, J=7.7 Hz, 1H), 7.12 (d, J=35.0 Hz, 1H), 4.56 (s, 2H), 4.33 (q, J=7.2 Hz, 2H), 3.47 (s, 3H), 1.35 (t, J=7.2 Hz, 3H).

### Step 3: Preparation of (Z)-2-fluoro-3-(6-(methoxymethyl)pyridin-2-yl)acrylic acid

Ethyl (Z)-2-fluoro-3-(6-(methoxymethyl)pyridin-2-yl)acrylate (59 mg, 0.25 mmol) was dissolved in MeOH (3 mL). The solution was cooled to 0° C. and sodium hydroxide solution (1.0 M, 0.25 mL, 0.25 mmol) was added. The reaction mixture was stirred for 2 h. The solution was concentrated to remove the methanol. Additional water (1 mL) was added, and the aqueous solution was cooled to 0° C. HCl solution (1.0 M, 0.28 mL, 0.28 mmol) was added dropwise. The solution was frozen and directly lyophilized to provide the desired product, sodium chloride adduct, as a white solid (quantitative yield assumed). LC/MS (ESI) m/z: [M+H]⁺ calcd for C₁₀H₁₁FNO₃ 212.1; found 211.8; ¹H NMR (500 MHz, DMSO-d₆) δ 7.89 (t, J=7.7 Hz, 1H), 7.71 (d, J=7.7 Hz, 1H), 7.38 (d, J=7.7 Hz, 1H), 6.87 (d, J=35.6 Hz, 1H), 4.50 (s, 2H), 3.37 (s, 3H).

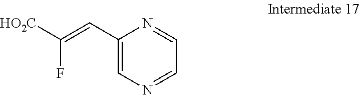 [SMILES: O=C(O)/C(F)=C/c1cnccn1]

### (Z)-2-fluoro-3-(pyrazin-2-yl)acrylic acid

### Step 1: Preparation of ethyl (E)-2-fluoro-3-(pyrazin-2-yl)acrylate

Ethyl 2-(diethoxyphosphoryl)-2-fluoroacetate (1.7 g, 7.0 mmol) was dissolved in THE (35 mL). The solution was cooled to 0° C. Sodium hydride (60% dispersion in mineral oil, 280 mg, 7.0 mmol) was added portion wise as a solid. The reaction mixture was stirred for 10 min and pyrazine-2-carbaldehyde (750 mg, 7.0 mmol) was added. The reaction mixture was allowed to warm to room temperature and was stirred for 1 h. The mixture was quenched by addition of saturated aqueous ammonium chloride solution (50 mL). The solution was diluted with water (50 mL) and EtOAc (100 mL). The layers were separated and the aqueous phase was further extracted with EtOAc (2×50 mL). The combined organic layers were dried over magnesium sulfate, filtered, and concentrated. The crude residue was purified by column chromatography (0→100% EtOAc/hexanes) to provide the desired product (6:1 E/Z as judged by 1H NMR, 930 mg, 4.8 mmol, 68% yield). LC/MS (ESI) m/z: [M+H]⁺ calcd for C₉H₁₀FN₂O₂ 197.1; found 197.0; E isomer reported: ¹H NMR (500 MHz, CDCl₃) δ 8.78 (d, J=1.4 Hz, 1H), 8.56 (dd, J=2.5, 1.4 Hz, 1H), 8.49 (d, J=2.5 Hz, 1H), 6.86 (d, J=19.1 Hz, 1H), 4.27 (q, J=7.2 Hz, 2H), 1.24 (t, J=7.2 Hz, 3H).

### Step 2: Preparation of ethyl (Z)-2-fluoro-3-(pyrazin-2-yl)acrylate

Ethyl (E)-2-fluoro-3-(pyrazin-2-yl)acrylate (930 mg, 4.8 mmol) was dissolved in toluene (50 mL) and iodine (240 mg, 0.95 mmol) was added. The reaction mixture was heated at 100° C. for 6 days. The solution was concentrated and purified by column chromatography (0-100% EtOAc/hexanes) to provide the desired product (710 mg, 3.6 mmol, 76% yield). LC/MS (ESI) m/z: [M+H]⁺ calcd for C₉H₁₀FN₂O₂ 197.1; found 197.0; ¹H NMR (500 MHz, CDCl₃) δ 9.10 (d, J=1.5 Hz, 1H), 8.63 (dd, J=2.5, 1.5 Hz, 1H), 8.51 (d, J=2.5 Hz, 1H), 7.10 (d, J=35.2 Hz, 1H), 4.39 (q, J=7.1 Hz, 2H), 1.40 (t, J=7.2 Hz, 3H).

### Step 3: Preparation of (Z)-2-fluoro-3-(pyrazin-2-yl)acrylic acid

Ethyl (Z)-2-fluoro-3-(pyrazin-2-yl)acrylate (710 mg, 3.6 mmol) was dissolved in MeOH (25 mL). The solution was cooled to 0° C. and sodium hydroxide solution (1.0 M, 3.6 mL, 3.6 mmol) was added. The reaction mixture was stirred for 2 h. The solution was concentrated to remove the methanol. The resulting aqueous solution was cooled to 0° C. HCl solution (1.0 M, 3.6 mL, 3.6 mmol) was added dropwise, and a precipitate formed. After stirring 10 min, the precipitate was collected by filtration and dried under vacuum to provide the desired product (570 mg, 3.4 mmol, 94% yield). LC/MS (ESI) m/z: [M+H]* calcd for C₇H₆FN₂O₂ 169.0; found 169.0; ¹H NMR (500 MHz, DMSO-d₆) δ 8.98 (d, J=1.1 Hz, 1H), 8.75 (dd, J=2.4, 1.1 Hz, 1H), 8.62 (d, J=2.4 Hz, 1H), 7.05 (d, J=34.9 Hz, 1H).

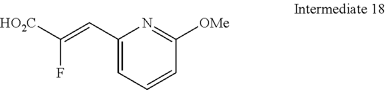 [SMILES: COc1cccc(/C=C(\F)C(=O)O)n1]

### (Z)-2-fluoro-3-(6-methoxypyridin-2-yl)acrylic acid

### Step 1: Preparation of ethyl (E)-2-fluoro-3-(6-methoxypyridin-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portion wise as a solid. The reaction mixture was stirred for 10 min and 6-methoxypicolinaldehyde (140 mg, 1.0 mmol) was added. The reaction mixture was allowed to warm to room temperature and was stirred for 1 h. The mixture was quenched by addition of saturated aqueous ammonium chloride solution (20 mL). The solution was diluted with water (20 mL) and EtOAc (10 mL). The layers were separated and the aqueous phase was further extracted with EtOAc (2×5 mL). The combined organic layers were dried over magnesium sulfate, filtered, and concentrated. The crude residue was purified by column chromatography (0-100% EtOAc/hexanes) to provide the desired product (6:1 E/Z as judged by 1H NMR, 170 mg, 0.75 mmol, 72% yield). LC/MS (ESI) m/z: [M+H]⁺ calcd for C₁₁H₁₃FNO₃ 226.1; found 225.9.

### Step 2: Preparation of ethyl (Z)-2-fluoro-3-(6-methoxypyridin-2-yl)acrylate

Ethyl (E)-2-fluoro-3-(6-methoxypyridin-2-yl)acrylate (170 mg, 0.75 mmol) was dissolved in toluene (5 mL) and iodine (190 mg, 0.75 mmol) was added. The reaction mixture was heated at 100° C. for 7 days. The solution was concentrated and purified by column chromatography (0→100% EtOAc/hexanes) to provide the desired product (75 mg, 0.33 mmol, 45% yield).; LC/MS (ESI) m/z: [M+H]⁺ calcd for C₁₁H₁₃FNO₃ 226.1; found 226.1; ¹H NMR (500 MHz, CDCL₃) δ 7.60 (dd, J=8.2, 7.4 Hz, 1H), 7.41 (d, J=7.4 Hz, 1H), 6.98 (d, J=34.7 Hz, 1H), 6.70 (d, J=8.2 Hz, 1H), 4.35 (q, J=7.2 Hz, 2H), 3.93 (s, 3H), 1.38 (t, J=7.2 Hz, 3H).

### Step 3: Preparation of (Z)-2-fluoro-3-(6-methoxypyridin-2-yl)acrylic acid

Ethyl (Z)-2-fluoro-3-(6-methoxypyridin-2-yl)acrylate (71 mg, 0.32 mmol) was dissolved in MeOH (3 mL). The solution was cooled to 0° C. and sodium hydroxide solution (1.0 M, 0.32 mL, 0.32 mmol) was added. The reaction mixture was stirred for 2 h. The solution was concentrated to remove the methanol. The resulting aqueous solution was cooled to 0° C. HCl solution (1.0 M, 0.32 mL, 0.32 mmol) was added dropwise, and a precipitate formed. After stirring 10 min, the precipitate was collected by filtration and dried under vacuum to provide the desired product (42 mg, 0.2 mmol, 67% yield). LC/MS (ESI) m/z: [M+H]⁺ calcd for C₉H₉FNO₃ 198.0; found 198.0; ¹H NMR (500 MHz, DMSO-d₆) δ 7.79 (dd, J=8.3, 7.0 Hz, 1H), 7.41 (d, J=7.4 Hz, 1H), 6.89 (d, J=34.5 Hz, 1H), 6.84 (d, J=8.3 Hz, 1H).

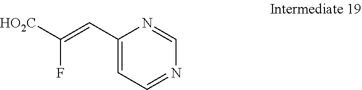 [SMILES: O=C(O)/C(F)=C/c1ccncn1]

### (Z)-2-fluoro-3-(pyrimidin-4-yl)acrylic acid

### Step 1: Preparation of ethyl (Z)-2-fluoro-3-(pyrimidin-4-yl)acrylate

Ethyl 2-(diethoxyphosphoryl)-2-fluoroacetate (1.1 g, 4.6 mmol) was dissolved in THE (11 mL). The solution was cooled to 0° C. Sodium hydride (60% dispersion in mineral oil, 185 mg, 4.6 mmol) was added portion wise as a solid. The reaction mixture was stirred for 10 min and pyrimidin-4-carbaldehyde (250 mg, 1.0 mmol) was added. The reaction mixture was allowed to warm to room temperature and was stirred for 1 h. The mixture was quenched by addition of saturated aqueous ammonium chloride solution (20 mL). The solution was diluted with water (20 mL) and EtOAc (10 mL). The layers were separated and the aqueous phase was further extracted with EtOAc (2×5 mL). The combined organic layers were dried over magnesium sulfate, filtered, and concentrated. The crude residue was purified by column chromatography (0→15% acetone/hexanes) to provide the desired product (40 mg, 0.2 mmol, 9% yield). LC/MS (ESI) m/z: [M+H]⁺ calcd for C₉H₁₀FN₂O₂ 197.1; found 197.0; ¹H NMR (500 MHz, CDCl₃) δ 9.27 (d, J=1.3 Hz, 1H), 8.82 (d, J=5.4 Hz, 1H), 7.79 (dd, J=5.3, 1.5 Hz, 1H), 7.03 (d, J=33.9 Hz, 1H), 4.39 (q, J=7.2 Hz, 2H), 1.40 (t, J=7.2 Hz, 3H).

### Step 2: Preparation of (Z)-2-fluoro-3-(pyrimidin-4-yl)acrylic acid

Ethyl (Z)-2-fluoro-3-(pyrimidin-4-yl)acrylate (20 mg, 0.1 mmol) was dissolved in MeOH (1 mL). The solution was cooled to 0° C. and sodium hydroxide solution (1.0 M, 0.1 mL, 0.1 mmol) was added. The reaction mixture was stirred for 2 h. The solution was concentrated to remove the methanol. The resulting aqueous solution was cooled to 0° C. HCl solution (1.0 M, 0.1 mL, 0.1 mmol) was added dropwise. The solution was frozen and directly lyophilized to provide the desired product, sodium chloride adduct (quantitative yield assumed). LC/MS (ESI) m/z: [M+H]⁺ calcd for C₇H₆FN₂O₂ 169.0; found 169.0.

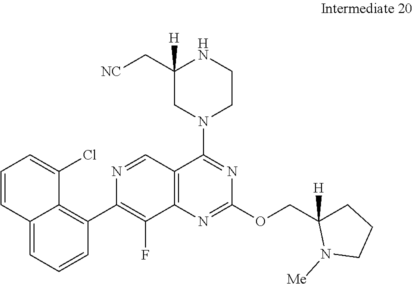 [SMILES: CN1CCC[C@H]1COc1nc(N2CCN[C@@H](CC#N)C2)c2cnc(-c3cccc4cccc(Cl)c34)c(F)c2n1]

### 2-((S)-4-(7-(8-chloronaphthalen-1-yl)-8-fluoro-2-(((S)-1-methylpyrrolidin-2-yl)methoxy)pyrido[4,3-d]pyrimidin-4-yl)piperazin-2-y)acetonitrile

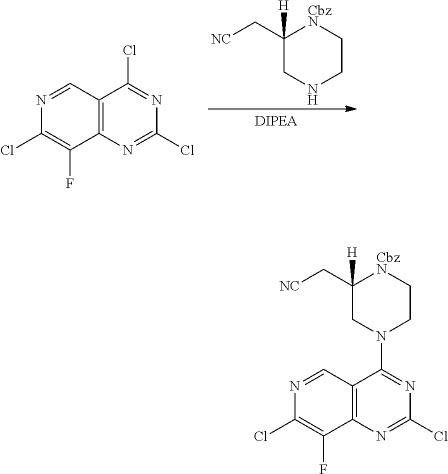 [SMILES: Fc1c(Cl)ncc2c(Cl)nc(Cl)nc12 ; N#CC[C@H]1CNCCN1C(=O)OCc1ccccc1 ; N#CC[C@H]1CN(c2nc(Cl)nc3c(F)c(Cl)ncc23)CCN1C(=O)OCc1ccccc1]

### Step 1: Preparation of benzyl (S)-2-(cyanomethyl)-4-(2,7-dichloro-8-fluoropyrido[4,3-d]pyrimidin-4-yl)piperazine-1-carboxylate

2,4,7-Trichloro-8-fluoropyrido[4,3-d]pyrimidine (1.2 g, 4.75 mmol) was suspended in DCM (24 mL), and the solution was cooled to −40° C. DIPEA (1.2 mL, 7.1 mmol) was added dropwise followed by benzyl (S)-2-(cyanomethyl)piperazine-1-carboxylate (1.2 g, 4.75 mmol) as a solution in DCM (10 mL). The reaction mixture was stirred for 30 min, was warmed to room temperature, and was concentrated. The crude residue was directly purified by column chromatography (0→50% EtOAc/hexanes) to provide the desired product (2.2 g, 4.64 mmol, 97%). LC/MS (ESI) m/z: [M+H]⁺ calcd for C₂₁H₁₈Cl₂FN₆O₂ 475.1; found 474.8; ¹H NMR (500 MHz, CDCl₃) δ 8.93 (s, 1H), 7.40-7.33 (m, 5H), 5.20 (d, J=12.3 Hz, 1H), 5.17 (d, J=12.3 Hz, 1H), 4.68-4.61 (m, 1H), 4.49-4.40 (m, 1H), 4.40-4.35 (m, 1H), 4.29-4.12 (m, 2H), 3.92-3.80 (m, 1H), 3.79-3.48 (m, 1H), 3.05-2.76 (m, 1H) 2.68 (dd, J=17.0, 5.0 Hz, 1H).

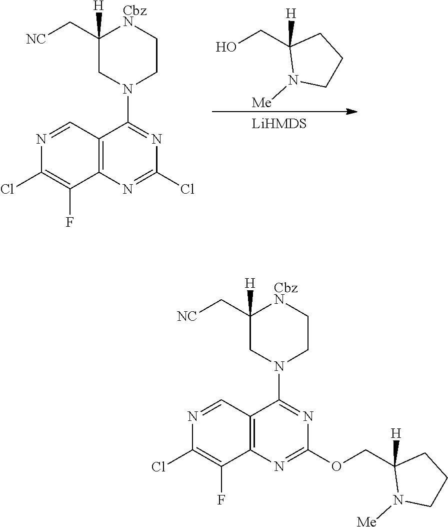 [SMILES: N#CC[C@H]1CN(c2nc(Cl)nc3c(F)c(Cl)ncc23)CCN1C(=O)OCc1ccccc1 ; CN1CCC[C@H]1CO ; CN1CCC[C@H]1COc1nc(N2CCN(C(=O)OCc3ccccc3)[C@@H](CC#N)C2)c2cnc(Cl)c(F)c2n1]

### Step 2: Preparation of benzyl (S)-4-(7-chloro-8-fluoro-2-(((S)-1-methylpyrrolidin-2-yl)methoxy)pyrido[4,3-d]pyrimidin-4-yl)-2-(cyanomethyl)piperazine-1-carboxylate

(S)-(1-Methylpyrrolidin-2-yl)methanol (100 mg, 0.88 mmol) was dissolved in THE (7.4 mL) and the solution was cooled to 0° C. LiHMDS solution (1.0 M in THF, 0.88 mL, 0.88 mmol) was added dropwise, and the resulting mixture was stirred for 10 min. Benzyl (S)-2-(cyanomethyl)-4-(2,7-dichloro-8-fluoropyrido[4,3-d]pyrimidin-4-yl)piperazine-1-carboxylate (350 mg, 0.74 mmol) was dissolved in THE (2 mL) and was added dropwise to the solution. The reaction mixture was allowed to warm to room temperature and was stirred for 16 h. The reaction mixture was partially concentrated, and then was directly purified by column chromatography (50-100% EtOAc w 5% Et₃N/hexanes) to provide the desired product (350 mg, 0.63 mmol, 85% yield) as a white foam. LC/MS (ESI) m/z: [M+H]⁺ calcd for C₂₇H₃₀C₁FN₇O₃ 554.2; found 554.1; ¹H NMR (500 MHz, CDCl₃) δ 8.79 (s, 1H), 7.41-7.33 (m, 5H), 5.22-5.16 (m, 2H), 4.66 (br s, 1H), 4.55 (dd, J=10.9, 4.8 Hz, 1H), 4.38 (dd, J=10.8, 6.1 Hz, 2H), 4.33-4.25 (m, 1H), 4.23-4.11 (m, 1H), 4.02-3.89 (m, 1H), 3.73-3.64 (m, 1H), 3.64-3.48 (m, 1H), 3.11 (br t, J=7.5 Hz, 1H), 3.00-2.77 (m, 1H), 2.74-2.65 (m, 2H), 2.49 (s, 3H), 2.33-2.25 (m, 1H), 2.08-2.00 (m, 1H), 1.90-1.72 (m, 3H).

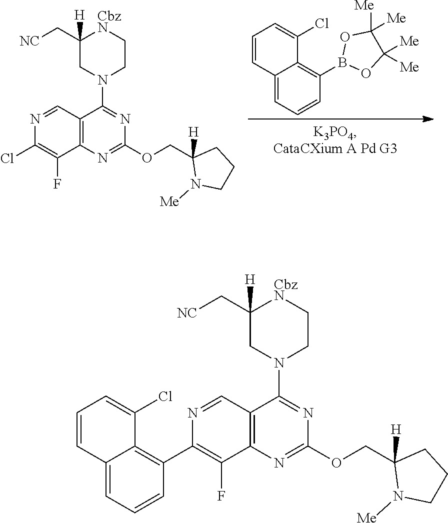 [SMILES: CN1CCC[C@H]1COc1nc(N2CCN(C(=O)OCc3ccccc3)[C@@H](CC#N)C2)c2cnc(Cl)c(F)c2n1 ; CC1(C)OB(c2cccc3cccc(Cl)c23)OC1(C)C ; CN1CCC[C@H]1COc1nc(N2CCN(C(=O)OCc3ccccc3)[C@@H](CC#N)C2)c2cnc(-c3cccc4cccc(Cl)c34)c(F)c2n1]

### Step 3: Preparation of benzyl (S)-4-(7-(8-chloronaphthalen-1-yl)-8-fluoro-2-(((S)-1-methylpyrrolidin-2-yl)methoxy)pyrido[4,3-d]pyrimidin-4-yl)-2-(cyanomethyl)piperazine-1-carboxylate

Benzyl (S)-4-(7-chloro-8-fluoro-2-(((S)-1-methylpyrrolidin-2-yl)methoxy)pyrido[4,3-d]pyrimidin-4-yl)-2-(cyanomethyl)piperazine-1-carboxylate (100 mg, 0.19 mmol), 2-(8-chloronaphthalen-1-yl)-4,4,5,5-tetramethyl-1,3,2-dioxaborolane (84 mg, 0.29 mmol), and methanesulfonato(diadamantyl-n-butylphosphino)-2′-amino-1,1′-biphenyl-2-yl)palladium(II) dichloromethane adduct [cataCXiumRTM A Palladacycle Gen. 3] (7 mg, 9.6 μmol) were combined as solids in a microwave vial. The vial was sealed. The atmosphere was evacuated and replaced with nitrogen. This process was performed three times. Degassed dioxane (2.9 mL) and potassium phosphate solution (2.0 M in water, 0.3 mL, 0.58 mmol) were added and the reaction mixture was heated at 100° C. in the microwave for 2 h. The reaction mixture was directly concentrated, and the crude residue was purified by column chromatography (0→100% EtOAc w/5% Et₃N/hexanes) to provide the desired product (80 mg, 0.12 mmol, 61% yield). LC/MS (ESI) m/z: [M+H]* calcd for C₃₇H₃₆C₁FN₇O₃ 680.3; found 680.4.

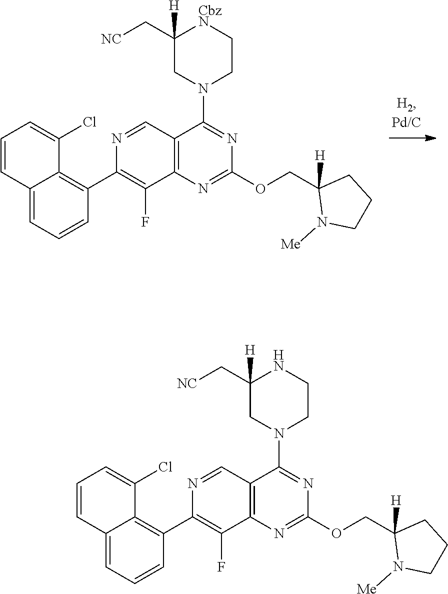 [SMILES: CN1CCC[C@H]1COc1nc(N2CCN(C(=O)OCc3ccccc3)[C@@H](CC#N)C2)c2cnc(-c3cccc4cccc(Cl)c34)c(F)c2n1 ; CN1CCC[C@H]1COc1nc(N2CCN[C@@H](CC#N)C2)c2cnc(-c3cccc4cccc(Cl)c34)c(F)c2n1]

### Step 4: Preparation of 2-((S)-4-(7-(8-chloronaphthalen-1-yl)-8-fluoro-2-(((S)-1-methylpyrrolidin-2-yl)methoxy)pyrido[4,3-d]pyrimidin-4-yl)piperazin-2-yl)acetonitrile

Benzyl (S)-4-(7-(8-chloronaphthalen-1-yl)-8-fluoro-2-(((S)-1-methylpyrrolidin-2-yl)methoxy)pyrido[4,3-d]pyrimidin-4-yl)-2-(cyanomethyl)piperazine-1-carboxylate (80 mg, 0.12 mmol) was dissolved in MeOH (2.4 mL) and palladium on carbon (10 wt. %, 63 mg, 0.06 mmol) was added. Hydrogen gas (balloon) was bubbled through the reaction mixture for 5 min, and the reaction mixture was kept under an atmosphere of hydrogen. After 30 min, the reaction mixture was filtered through a pad of diatomaceous earth (Celite©) and was concentrated. The crude residue, containing an unquantified mixture of desired product and dehalogenated product (2-((S)-4-(8-fluoro-2-(((S)-1-methylpyrrolidin-2-yl)methoxy)-7-(naphthalen-1-yl)pyrido[4,3-d]pyrimidin-4-yl)piperazin-2-yl)acetonitrile), was used directly in the next step, without additional purification. LC/MS (ESI) m/z: [M+H]⁺ calcd for C₂₈H₃₁ClFN₇O 546.2; found 546.1.

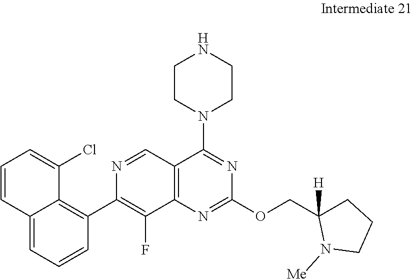 [SMILES: CN1CCC[C@H]1COc1nc(N2CCNCC2)c2cnc(-c3cccc4cccc(Cl)c34)c(F)c2n1]

### (S)-7-(8-chloronaphthalen-1-yl)-8-fluoro-2-((I-methylpyrrolidin-2-yl)methoxy)-4-(piperazin-1-yl)pyrido[4,3-d]pyrimidine

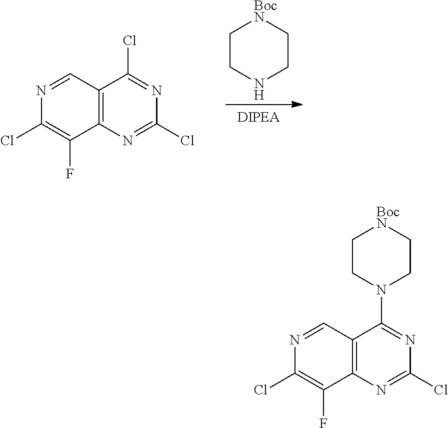 [SMILES: Fc1c(Cl)ncc2c(Cl)nc(Cl)nc12 ; CC(C)(C)OC(=O)N1CCNCC1 ; CC(C)(C)OC(=O)N1CCN(c2nc(Cl)nc3c(F)c(Cl)ncc23)CC1]

### Step 1: Preparation of tert-butyl 4-(2,7-dichloro-8-fluoropyrido[4,3-d]pyrimidin-4-yl)piperazine-1-carboxylate

2,4,7-Trichloro-8-fluoropyrido[4,3-d]pyrimidine (1 g, 4.0 mmol) was suspended in DCM (20 mL), and the solution was cooled to −40° C. DIPEA (1.0 mL, 5.9 mmol) was added followed by tert-butyl piperazine-1-carboxylate (0.74 g, 4.0 mmol) as a solution in DCM (5 mL). After 30 min, the reaction mixture was warmed to room temperature and was partially concentrated. The crude residue was purified by column chromatography (0→50% EtOAc/hexanes) to provide the desired product (1.4 g, 3.6 mmol, 90% yield) as an off-white solid. LC/MS (ESI) m/z: [M+H]⁺ calcd for C₁₆H₁₉C₁₂FN₅O₂ 402.1; found 401.9; ¹H NMR (500 MHz, CDCl₃) δ 8.87 (s, 1H), 4.09-4.02 (m, 4H), 3.72-3.64 (m, 4H), 1.50 (s, 9H).

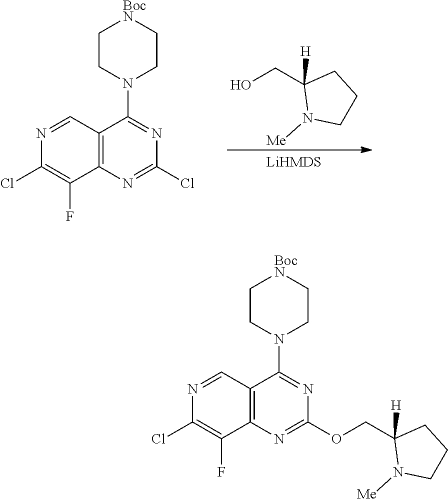 [SMILES: CC(C)(C)OC(=O)N1CCN(c2nc(Cl)nc3c(F)c(Cl)ncc23)CC1 ; CN1CCC[C@H]1CO ; CN1CCC[C@H]1COc1nc(N2CCN(C(=O)OC(C)(C)C)CC2)c2cnc(Cl)c(F)c2n1]

### Step 2: Preparation of tert-butyl (S)-4-(7-chloro-8-fluoro-2-((I-methylpyrrolidin-2-yl)methoxy)pyrido[4,3-d]pyrimidin-4-yl)piperazine-1-carboxylate

(S)-(1-Methylpyrrolidin-2-yl)methanol (0.6 g, 5.2 mmol) was dissolved in THE (17.4 mL), and the solution was cooled to 0° C. LiHMDS solution (1.0 M in THF, 5.2 mL, 5.2 mmol) was added dropwise, and the resulting mixture was stirred for 10 min. tert-Butyl 4-(2,7-dichloro-8-fluoropyrido[4,3-d]pyrimidin-4-yl)piperazine-1-carboxylate (1.4 g, 3.5 mmol) was suspended in THE (10 mL) and was added dropwise to the solution. The reaction mixture was allowed to warm to room temperature and was stirred for 16 h. The reaction mixture was partially concentrated, and the crude residue was purified by column chromatography (50→100% EtOAc with 5% Et₃N/hexanes) to provide the desired product (1.5 g, 3.1 mmol, 90% yield). LC/MS (ESI) m/z: [M+H]⁺ calcd for C₂₂H₃₁C₁FN₆O₃ 481.2; found 481.1; ¹H NMR (500 MHz, CDCl₃) δ 8.75 (s, 1H), 4.53 (dd, J=10.7, 4.6 Hz, 1H), 4.34 (dd, J=10.7, 6.7 Hz, 1H), 3.99-3.90 (m, 4H), 3.69-3.60 (m, 4H), 3.12-3.07 (m, 1H), 2.74-2.67 (m, 1H), 2.49 (s, 3H), 2.28 (td, J=9.5, 6.9 Hz, 1H), 2.09-2.00 (m, 1H), 1.89-1.73 (m, 3H), 1.49 (s, 9H).

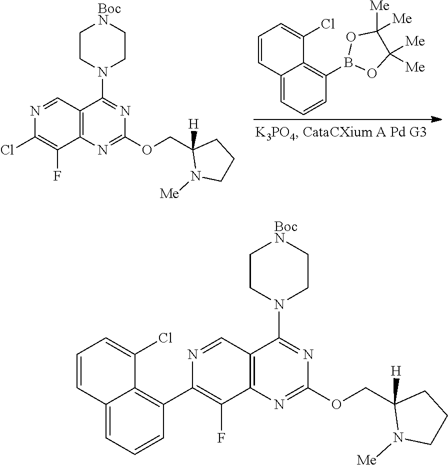 [SMILES: CN1CCC[C@H]1COc1nc(N2CCN(C(=O)OC(C)(C)C)CC2)c2cnc(Cl)c(F)c2n1 ; CC1(C)OB(c2cccc3cccc(Cl)c23)OC1(C)C ; CN1CCC[C@H]1COc1nc(N2CCN(C(=O)OC(C)(C)C)CC2)c2cnc(-c3cccc4cccc(Cl)c34)c(F)c2n1]

### Step 3: Preparation of tert-butyl (S)-4-(7-(8-chloronaphthalen-1-yl)-8-fluoro-2-((1-methylpyrrolidin-2-yl)methoxy)pyrido[4,3-d]pyrimidin-4-yl)piperazine-1-carboxylate

tert-Butyl (S)-4-(7-chloro-8-fluoro-2-((1-methylpyrrolidin-2-yl)methoxy)pyrido[4,3-d]pyrimidin-4-yl)piperazine-1-carboxylate (145 mg, 0.30 mmol), 2-(8-chloronaphthalen-1-yl)-4,4,5,5-tetramethyl-1,3,2-dioxaborolane (105 mg, 0.36 mmol) and methanesulfonato(diadamantyl-n-butylphosphino)-2′-amino-1,1′-biphenyl-2-yl)palladium(II) dichloromethane adduct [cataCXiumRTM A Palladacycle Gen. 3] (22 mg, 0.03 mmol) were combined as solids in a microwave vial. The vial was sealed. The atmosphere was evacuated and replaced with nitrogen. This process was performed three times. Degassed dioxane (5 mL) and potassium phosphate solution (2.0 M in water, 0.45 mL, 0.90 mmol) were added and the reaction mixture was heated at 100° C. in the microwave for 2 h. The reaction mixture was directly concentrated, and the crude residue was purified by column chromatography (0→100% EtOAc w/5% Et₃N/hexanes) to provide the desired product (84 mg, 0.14 mmol, 45% yield). LC/MS (ESI) m/z: [M+H]⁺ calcd for C₃₂H₃₇C₁FN₆O₃ 607.3; found 607.2.

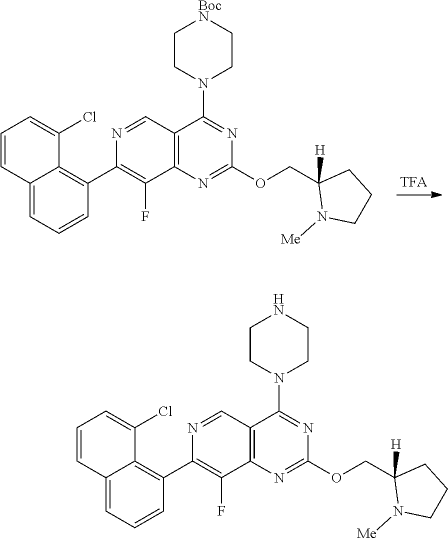 [SMILES: CN1CCC[C@H]1COc1nc(N2CCN(C(=O)OC(C)(C)C)CC2)c2cnc(-c3cccc4cccc(Cl)c34)c(F)c2n1 ; CN1CCC[C@H]1COc1nc(N2CCNCC2)c2cnc(-c3cccc4cccc(Cl)c34)c(F)c2n1]

### Step 4: Preparation of (S)-7-(8-chloronaphthalen-1-yl)-8-fluoro-2-((I-methylpyrrolidin-2-yl)methoxy)-4-(piperazin-1-yl)pyrido[4,3-d]pyrimidine

tert-Butyl (S)-4-(7-(8-chloronaphthalen-1-yl)-8-fluoro-2-((1-methylpyrrolidin-2-yl)methoxy)pyrido[4,3-d]pyrimidin-4-yl)piperazine-1-carboxylate (15 mg, 0.025 mmol) was dissolved in DCM (1 mL), and TFA (0.25 mL, 3.2 mmol) was added. The reaction mixture was stirred at room temperature for 1.5 h. The reaction mixture was directly concentrated, and the crude residue was used directly in the next step, without additional purification. LC/MS (ESI) m/z: [M+H]⁺ calcd for C₂₇H₂₉C₁FN60 507.2 found 507.1.

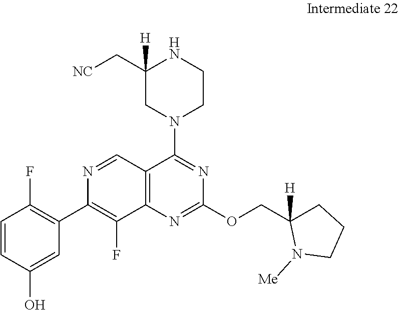 [SMILES: CN1CCC[C@H]1COc1nc(N2CCN[C@@H](CC#N)C2)c2cnc(-c3cc(O)ccc3F)c(F)c2n1]

### 2-((S)-4-(8-fluoro-7-(2-fluoro-5-hydroxyphenyl)-2-(((S)-1-methylpyrrolidin-2-yl)methoxy)pyrido(4,3-d₁pyrimidin-4-yl)-1-methylpiperazin-2-yl)acetonitrile

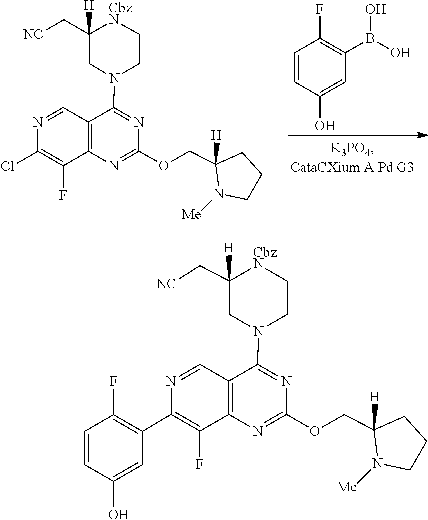 [SMILES: CN1CCC[C@H]1COc1nc(N2CCN(C(=O)OCc3ccccc3)[C@@H](CC#N)C2)c2cnc(Cl)c(F)c2n1 ; OB(O)c1cc(O)ccc1F ; CN1CCC[C@H]1COc1nc(N2CCN(C(=O)OCc3ccccc3)[C@@H](CC#N)C2)c2cnc(-c3cc(O)ccc3F)c(F)c2n1]

### Step 1: Preparation of benzyl (S)-2-(cyanomethyl)-4-(8-fluoro-7-(2-fluoro-5-hydroxyphenyl)-2-(((S)-1-methylpyrrolidin-2-yl)methoxy)pyrido[4,3-d]pyrimidin-4-yl)piperazine-1-carboxylate

Benzyl (S)-4-(7-chloro-8-fluoro-2-(((S)-1-methylpyrrolidin-2-yl)methoxy)pyrido[4,3-d]pyrimidin-4-yl)-2-(cyanomethyl)piperazine-1-carboxylate (32 mg, 0.058 mmol), and Methanesulfonato(diadamantyl-n-butylphosphino)-2′-amino-1,1′-biphenyl-2-yl)palladium(II) dichloromethane adduct [cataCXiumRTM A Palladacycle Gen. 3] (4.2 mg, 5.8 μmol) were combined as solids in a microwave vial. The vial was sealed. The atmosphere was evacuated and replaced with nitrogen. This process was performed three times. Degassed dioxane (0.9 mL)and potassium phosphate solution (2.0 M in water, 0.1 mL, 0.17 mmol) were added and the reaction mixture was heated at 100° C. in the microwave for 4 h. The reaction mixture was directly concentrated, and the crude residue was purified by column chromatography (80→100% EtOAc with 5% Et₃N/hexanes→20% MeOH/EtOAc with 5% Et₃N) to provide the desired product (30 mg, 0.048 mmol, 82% yield). LC/MS (ESI) m/z: [M+H]⁺ calcd for C₃₃H₃₄F₂N₇O₄ 630.3; found 630.2; ¹H NMR (500 MHz, CDCL₃) δ 8.93 (s, 1H), 7.43-7.30 (m, 6H), 7.05-6.86 (m, 3H), 5.19 (s, 2H), 4.71-4.56 (m, 2H), 4.52-4.44 (m, 1H), 4.40-4.32 (m, 1H), 4.28-4.18 (m, 1H), 4.15-3.96 (m, 1H), 3.70-3.54 (m, 2H), 3.50-3.39 (m, 1H), 3.36-3.23 (m, 1H), 3.04-2.93 (m, 1H), 2.87-2.72 (m, 1H), 2.70-2.61 (m, 4H), 2.54-2.44 (m, 1H), 2.15-2.08 (m, 1H), 2.05-1.83 (m, 3H).

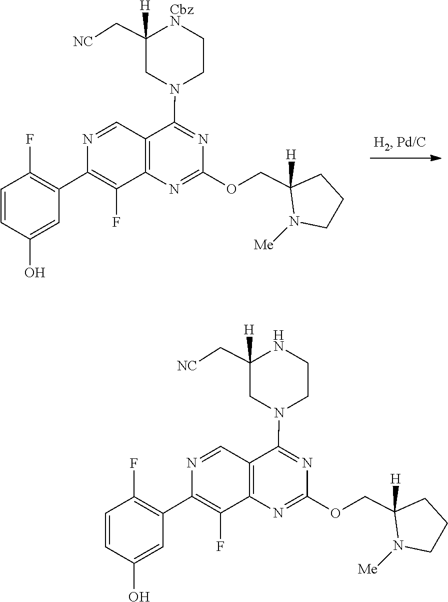 [SMILES: CN1CCC[C@H]1COc1nc(N2CCN(C(=O)OCc3ccccc3)[C@@H](CC#N)C2)c2cnc(-c3cc(O)ccc3F)c(F)c2n1 ; CN1CCC[C@H]1COc1nc(N2CCN[C@@H](CC#N)C2)c2cnc(-c3cc(O)ccc3F)c(F)c2n1]

### Step 2: Preparation of 2-((S)-4-(8-fluoro-7-(2-fluoro-5-hydroxyphenyl)-2-(((S)-1-methylpyrrolidin-2-yl)methoxy)pyrido[4,3-d]pyrimidin-4-yl)piperazin-2-yl)acetonitrile

Benzyl (S)-2-(cyanomethyl)-4-(8-fluoro-7-(2-fluoro-5-hydroxyphenyl)-2-(((S)-1-methylpyrrolidin-2-yl)methoxy)pyrido[4,3-d]pyrimidin-4-yl)piperazine-1-carboxylate (30 mg, 0.048 mmol) was dissolved in MeOH (1.0 mL) and palladium on carbon (10 wt. %, 25 mg, 0.024 mmol) was added. Hydrogen gas was bubbled through the solution for 5 min then the reaction mixture was kept under an atmosphere of hydrogen (balloon). After 30 min, the reaction mixture was filtered through a pad of diatomaceous earth (Celite©) and was directly concentrated to provide the desired product (quantitative yield assumed). LC/MS (ESI) m/z: [M+H]⁺ calcd for C₂₅H₂₈F₂N₇O₂ 496.2; found 496.3.

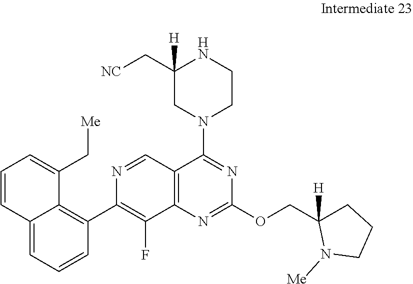 [SMILES: CCc1cccc2cccc(-c3ncc4c(N5CCN[C@@H](CC#N)C5)nc(OC[C@@H]5CCCN5C)nc4c3F)c12]

### 2-((S)-4-(7-(8-ethylnaphthalen-1-yl)-8-fluoro-2-(((S)-1-methylpyrrolidin-2-yl)methoxy)pyrido[4,3-d]pyrimidin-4-yl)piperazin-2-yl)acetonitrile

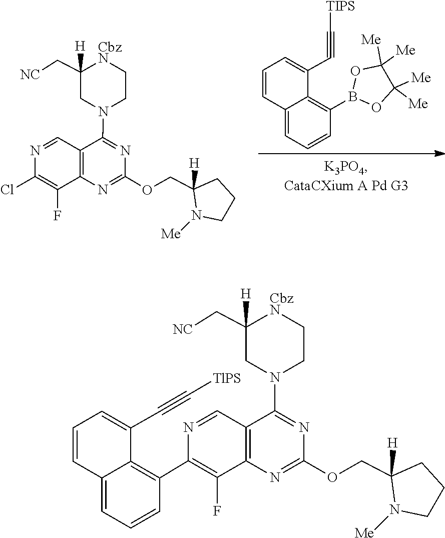

### Step 1: Preparation of benzyl (S)-2-(cyanomethyl)-4-(8-fluoro-2-(((S)-1-methylpyrrolidin-2-yl)methoxy)-7-(8-((triisopropylsilyl)ethynyl)naphthalen-1-yl)pyrido[4,3-d]pyrimidin-4-yl)piperazine-1-carboxylate

Benzyl (S)-4-(7-chloro-8-fluoro-2-(((S)-1-methylpyrrolidin-2-yl)methoxy)pyrido[4,3-d]pyrimidin-4-yl)-2-(cyanomethyl)piperazine-1-carboxylate (32 mg, 0.058 mmol), triisopropyl((8-(4,4,5,5-tetramethyl-1,3,2-dioxaborolan-2-yl)naphthalen-1-yl)ethynyl)silane ([this material prepared according to procedures detailed in WO 2022031678A1] 38 mg, 0.087 mmol), and methanesulfonato(diadamantyl-n-butylphosphino)-2′-amino-1,1′-biphenyl-2-yl)palladium(II) dichloromethane adduct, [cataCXium© A Palladacycle Gen. 3] (4.2 mg, 5.8 μmol) were combined as solids in a microwave vial. The vial was sealed. The atmosphere was evacuated and replaced with nitrogen. This process was performed three times. Degassed dioxane (0.9 mL) and potassium phosphate solution (2.0 M in water, 90 μl, 0.17 mmol) were added and the reaction mixture was heated at 100° C. in the microwave for 4 h. The reaction mixture was directly concentrated, and the crude residue was purified by column chromatography (20→100% EtOAc with 5% Et₃N/hexanes) to provide the desired product (35 mg, 0.04 mmol, 73% yield). LC/MS (ESI) m/z: [M+H]⁺ calcd for C₄₈H₅₆FN₇O₃Si 826.4; found 826.4; ¹H NMR (500 MHz, CDCl₃) δ 9.13-9.02 (m, 1H), 7.99-7.90 (m, 2H), 7.82 (d, J=6.7 Hz, 1H), 7.62-7.44 (m, 3H), 7.42-7.33 (m, 5H), 5.27-5.13 (m, 2H), 4.76-4.69 (m, 1H), 4.64-4.52 (m, 1H), 4.43-4.18 (m, 3H), 4.04-3.56 (m, 2H), 3.10 (br t, J=7.7 Hz, 1H), 2.89-2.55 (m, 3H), 2.51-2.46 (m, 3H), 2.33-2.23 (m, 1H), 2.10-2.01 (m, 1H), 1.91-1.71 (m, 4H), 0.95-0.82 (m, 18H), 0.61-0.49 (m, 3H).

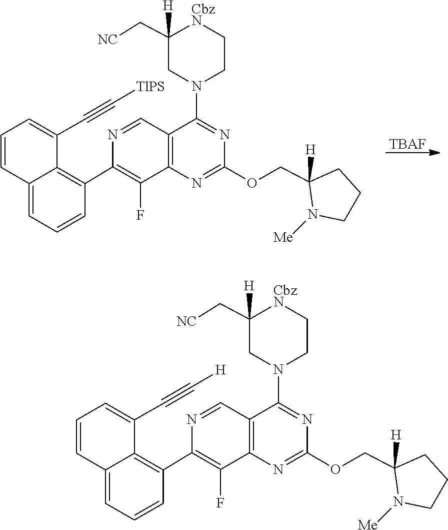

### Step 2: Preparation of benzyl (S)-2-(cyanomethyl)-4-(7-(8-ethynylnaphthalen-1-yl)-8-fluoro-2-(((S)-1-methylpyrrolidin-2-yl)methoxy)pyrido[4,3-d]pyrimidin-4-yl)piperazine-1-carboxylate

Benzyl (S)-2-(cyanomethyl)-4-(8-fluoro-2-(((S)-1-methylpyrrolidin-2-yl)methoxy)-7-(8-((triisopropylsilyl)ethynyl)naphthalen-1-yl)pyrido[4,3-d]pyrimidin-4-yl)piperazine-1-carboxylate (35 mg, 0.042 mmol) was dissolved in THE (0.8 mL) and tetra-n-butylammonium fluoride solution (1.0 M in THF, 40 μL, 0.04 mmol) was added dropwise. The reaction mixture was stirred at room temperature for 10 min. The solution was directly concentrated, and the crude residue was used directly in the next step, without additional purification (quantitative yield assumed) LC/MS (ESI) m/z: [M+H]⁺ calcd for C₃₉H₃₇FN₇O₃ 670.3; found 670.5.

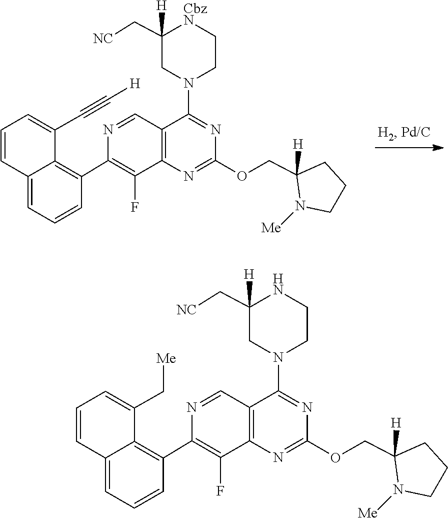 [SMILES: C#Cc1cccc2cccc(-c3ncc4c(N5CCN(C(=O)OCc6ccccc6)[C@@H](CC#N)C5)nc(OC[C@@H]5CCCN5C)nc4c3F)c12 ; CCc1cccc2cccc(-c3ncc4c(N5CCN[C@@H](CC#N)C5)nc(OC[C@@H]5CCCN5C)nc4c3F)c12]

### Step 3: Preparation of 2-((S)-4-(7-(8-ethylnaphthalen-1-yl)-8-fluoro-2-(((S)-1-methylpyrrolidin-2-yl)methoxy)pyrido[4,3-d]pyrimidin-4-yl)piperazin-2-yl)acetonitrile

Benzyl (S)-2-(cyanomethyl)-4-(7-(8-ethynylnaphthalen-1-yl)-8-fluoro-2-(((S)-1-methylpyrrolidin-2-yl)methoxy)pyrido[4,3-d]pyrimidin-4-yl)piperazine-1-carboxylate (28 mg assumed, 0.042 mmol) was dissolved in MeOH (0.8 mL) and palladium on carbon (10 wt. %, 22 mg, 0.021 mmol) was added. Hydrogen gas was bubbled through the solution for 5 min then the reaction mixture was kept under an atmosphere of hydrogen (balloon) for 1.5 h. The reaction mixture was filtered through a plug of diatomaceous earth (Celite©) and was concentrated. The crude residue was used directly in the next step, without additional purification (quantitative yield assumed). LC/MS (ESI) m/z: [M+H]⁺ calcd for C₃₁H₃₅FN₇O 540.3; found 540.4.

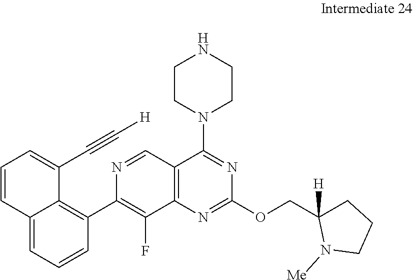 [SMILES: C#Cc1cccc2cccc(-c3ncc4c(N5CCNCC5)nc(OC[C@@H]5CCCN5C)nc4c3F)c12]

### 2-((S)-4-(7-(8-ethylnaphthalen-1-yl)-8-fluoro-2-(((S)-1-methylpyrrolidin-2-yl)methoxy)pyrido[4,3-d]pyrimidin-4-yl)piperazin-2-yl)acetonitrile

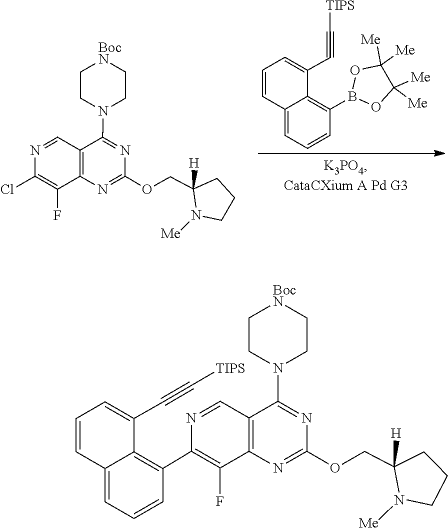

### Step 1: Preparation of(S)-7-(8-ethynylnaphthalen-1-yl)-8-fluoro-2-((I-methylpyrrolidin-2-yl)methoxy)-4-(piperazin-1-yl)pyrido[4,3-d]pyrimidine

tert-Butyl (S)-4-(7-chloro-8-fluoro-2-((1-methylpyrrolidin-2-yl)methoxy)pyrido[4,3-d]pyrimidin-4-yl)piperazine-1-carboxylate (70 mg, 0.15 mmol), triisopropyl((8-(4,4,5,5-tetramethyl-1,3,2-dioxaborolan-2-yl)naphthalen-1-yl)ethynyl)silane (95 mg, 0.22 mmol), and methanesulfonato(diadamantyl-n-butylphosphino)-2′-amino-1,1′-biphenyl-2-yl)palladium(II) dichloromethane adduct, [cataCXium© A Palladacycle Gen. 3] (11 mg, 15 μmol) were combined as solids in a microwave vial. The vial was sealed. The atmosphere was evacuated and replaced with nitrogen. This process was performed three times. Degassed dioxane (1.5 mL) and potassium phosphate solution (2.0 M in water, 220 μl, 0.44 mmol) were added and the reaction mixture was heated at 100° C. in the microwave for 4 h. The reaction mixture was directly concentrated, and the crude residue was purified by column chromatography (20→100% EtOAc with 5% Et₃N/hexanes) to provide the desired product (100 mg, 0.13 mmol, 91% yield). LC/MS (ESI) m/z: [M+H]⁺ calcd for C₄₃H₅SFN₆O₃Si 753.4; found 753.4.

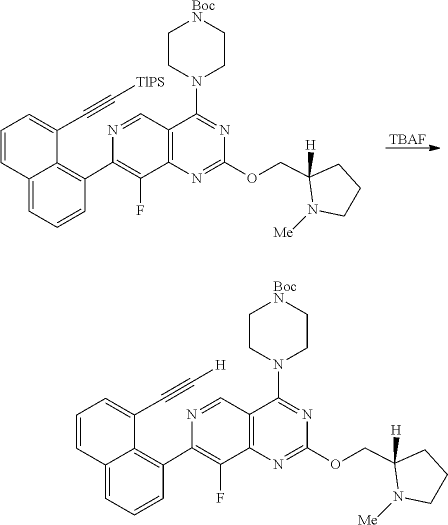

### Step 2: Preparation of tert-butyl (S)-4-(7-(8-ethynylnaphthalen-1-yl)-8-fluoro-2-((1-methylpyrrolidin-2-yl)methoxy)pyrido[4,3-d]pyrimidin-4-yl)piperazine-1-carboxylate

tert-Butyl (S)-4-(8-fluoro-2-((1-methylpyrrolidin-2-yl)methoxy)-7-(8-((triisopropylsilyl)ethynyl)naphthalen-1-yl)pyrido[4,3-d]pyrimidin-4-yl)piperazine-1-carboxylate (100 mg, 0.133 mmol) was dissolved in THE (1.3 mL) and tetra-n-butylammonium fluoride solution (1.0 M in THF, 160 μL, 0.16 mmol) was added dropwise. The reaction mixture was stirred at room temperature for 10 min. The solution was directly concentrated, and the crude residue was used directly in the next step, without additional purification (quantitative yield assumed). LC/MS (ESI) m/z: [M+H]⁺ calcd for C₃₄H₃₈FN₆O₃ 597.3; found 597.4.

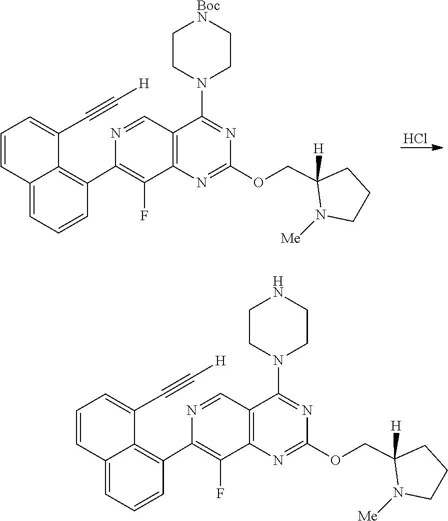 [SMILES: C#Cc1cccc2cccc(-c3ncc4c(N5CCN(C(=O)OC(C)(C)C)CC5)nc(OC[C@@H]5CCCN5C)nc4c3F)c12 ; Cl ; C#Cc1cccc2cccc(-c3ncc4c(N5CCNCC5)nc(OC[C@@H]5CCCN5C)nc4c3F)c12]

### Step 3: Preparation of (S)-7-(8-ethynylnaphthalen-1-yl)-8-fluoro-2-((I-methylpyrrolidin-2-yl)methoxy)-4-(piperazin-1-yl)pyrido[4,3-d]pyrimidine

tert-Butyl (S)-4-(7-(8-ethynylnaphthalen-1-yl)-8-fluoro-2-((1-methylpyrrolidin-2-yl)methoxy)pyrido[4,3-d]pyrimidin-4-yl)piperazine-1-carboxylate (10 mg, 0.017 mmol) was dissolved in MeCN (300 μL) and the solution was cooled to 0° C. HCl solution (4.0 M in dioxane, 42 μL, 0.168 mmol) was added dropwise and the reaction mixture was stirred for 1 h. The reaction mixture was quenched with NaOH solution (1.0 M, 170 μL, 0.17 mmol) and the solution was concentrated. The material was used directly in the next step, without additional purification. LC/MS (ESI) m/z: [M+H]⁺ calcd for C₂₉H₃₀FN₆O497.2; found 497.3.

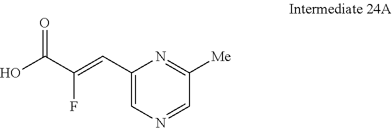 [SMILES: Cc1cncc(/C=C(\F)C(=O)O)n1]

### (Z)-2-fluoro-3-(6-methylpyrazin-2-yl)acrylic acid

### Step 1: Preparation of ethyl (E)-2-fluoro-3-(6-methylpyrazin-2-yl)acrylate

Ethyl 2-(diethoxyphosphoryl)-2-fluoroacetate (250 mg, 1.0 mmol) was dissolved in THE (5 mL). The solution was cooled to 0° C. Sodium hydride (60% dispersion in mineral oil, 41 mg, 1.0 mmol) was added portion wise as a solid. The reaction mixture was stirred for 10 min and 6-methylpyrazine-2-carbaldehyde (125 mg, 1.0 mmol) was added. The reaction mixture was allowed to warm to room temperature and was stirred for 1 h. The mixture was quenched by addition of saturated aqueous ammonium chloride solution (5 mL). The solution was diluted with water (5 mL) and EtOAc (10 mL). The layers were separated and the aqueous phase was further extracted with EtOAc (2×5 mL). The combined organic layers were dried over magnesium sulfate, filtered, and concentrated. The crude residue was purified by column chromatography (0→100% EtOAc/hexanes) to provide the desired product (>15:1 E/Z as judged by 1H NMR, 120 mg, 0.56 mmol, 55% yield). LC/MS (ESI) m/z: [M+H]⁺ calcd for C₁₀H₁₂FN₂O₂ 211.1; found 210.9; ¹H NMR (500 MHz, CDCl₃) δ 8.56 (s, 1H), 8.36 (s, 1H), 6.81 (d, J=19.1 Hz, 1H), 4.26 (q, J=7.2 Hz, 2H), 2.55 (s, 3H), 1.24 (t, J=7.1 Hz, 3H).

### Step 2: Preparation of ethyl (Z)-2-fluoro-3-(6-methylpyrazin-2-yl)acrylate

Ethyl (E)-2-fluoro-3-(6-methylpyrazin-2-yl)acrylate (120 mg, 0.56 mmol) was dissolved in toluene (7 mL) and iodine (14 mg, 0.06 mmol) was added. The reaction mixture was heated at 100° C. for 5 days. The solution was concentrated and purified by column chromatography (5→100% EtOAc/hexanes) to provide the desired product (73 mg, 0.35 mmol, 62% yield). LC/MS (ESI) m/z: [M+H]⁺ calcd for C₁₀H₁₂FN₂O₂ 211.1; found 210.9; ¹H NMR (500 MHz, CDCl₃) δ 8.92 (s, 1H), 8.39 (s, 1H), 7.07 (d, J=35.6 Hz, 1H), 4.37 (q, J=7.2 Hz, 2H), 2.58 (s, 3H), 1.38 (t, J=7.2 Hz, 3H).

### Step 3: Preparation of (Z)-2-fluoro-3-(6-methylpyrazin-2-yl)acrylic acid

Ethyl (Z)-2-fluoro-3-(6-methylpyrazin-2-yl)acrylate (73 mg, 0.35 mmol) was dissolved in MeOH (3 mL). The solution was cooled to 0° C. and sodium hydroxide solution (1.0 M, 0.35 mL, 3.5 mmol) was added. The reaction mixture was stirred for 2 h. The solution was concentrated to remove the methanol. The resulting aqueous solution was cooled to 0° C. HCl solution (1.0 M, 0.35 mL, 0.35 mmol) was added dropwise, and a precipitate formed. After stirring 10 min, the precipitate was collected by filtration and dried under vacuum to provide the desired product (34 mg, 0.19 mmol, 54% yield). LC/MS (ESI) m/z: [M+H]⁺ calcd for C₈H₈FN₂O₂ 183.0; found 182.8; ¹H NMR (500 MHz, DMSO-d₆) δ 8.79 (s, 1H), 8.52 (s, 1H), 6.98 (d, J=35.0 Hz, 1H), 2.52 (s, 3H).

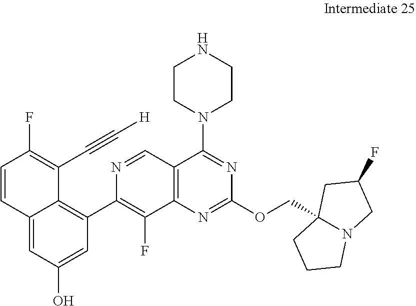 [SMILES: C#Cc1c(F)ccc2cc(O)cc(-c3ncc4c(N5CCNCC5)nc(OC[C@@]56CCCN5C[C@H](F)C6)nc4c3F)c12]

### 5-ethynyl-6-fluoro-4-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol

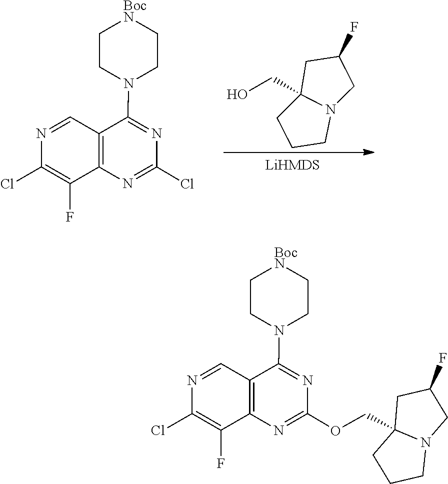 [SMILES: CC(C)(C)OC(=O)N1CCN(c2nc(Cl)nc3c(F)c(Cl)ncc23)CC1 ; OC[C@@]12CCCN1C[C@H](F)C2 ; CC(C)(C)OC(=O)N1CCN(c2nc(OC[C@@]34CCCN3C[C@H](F)C4)nc3c(F)c(Cl)ncc23)CC1]

### Step 1: Preparation of tert-butyl 4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate

((2R, 7aS)-2-Fluorotetrahydro-1H-pyrrolizin-7a(5H)-yl)methanol, HCl salt (1.9 g, 9.5 mmol) was suspended in THE (70 mL). The mixture was cooled to 0° C. and LiHMDS solution (1.0 M in THF, 22 mL, 22 mmol) was added dropwise. Once all solid had gone into solution (5 min), tert-Butyl 4-(2,7-dichloro-8-fluoropyrido[4,3-d]pyrimidin-4-yl)piperazine-1-carboxylate (3.5 g, 8.7 mmol) was added as a solution in THE (10 mL). The reaction mixture was allowed to warm to room temperature and was stirred for 16 h. The reaction mixture was partially concentrated, and the crude residue was purified by column chromatography (50→100% EtOAc with 5% Et₃N/hexanes) to provide the desired product (3.0 g, 5.7 mmol, 66% yield) as a white foam. LC/MS (ESI) m/z: [M+H]⁺ calcd for C₂₄H₃₂C₁F₂N₆O₃ 525.2; found 525.5; ¹H NMR (500 MHz, CDCl₃) δ 8.74 (s, 1H), 0.00 (d, J=54.0 Hz, 1H), 4.26 (d, J=10.5 Hz, 1H), 4.18 (d, J=10.5 Hz, 1H), 3.99-3.89 (m, 4H), 3.69-3.59 (m, 4H), 3.31-3.12 (m, 3H), 3.01-2.93 (m, 1H), 2.25-2.18 (m, 1H), 2.14 (br dd, J=14.7, 4.1 Hz, 1H), 1.99-1.80 (m, 4H), 1.49 (s, 9H).

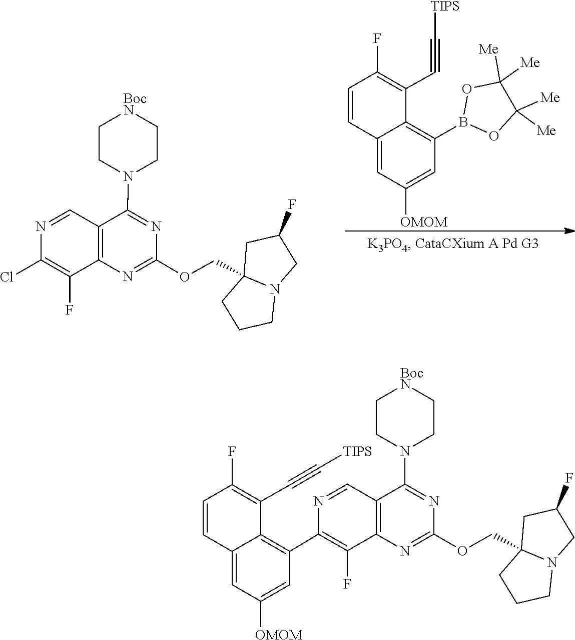

### Step 2: Preparation of tert-butyl 4-(8-fluoro-7-(7-fluoro-3-(methoxymethoxy)-8-((triisopropylsilyl)ethynyl)naphthalen-1-yl)-2-(((2R, 7aS)-2-fluorotetrahydro-1H-pyrrolizin-7a(5H)-yl)methoxy)pyrido[4,3-d]pyrimidin-4-yl)piperazine-1-carboxylate

tert-Butyl 4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (300 mg, 0.57 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (440 mg, 0.86 mmol), and methanesulfonato(diadamantyl-n-butylphosphino)-2′-amino-1,1′-biphenyl-2-yl)palladium(II) dichloromethane adduct [CataCXiumRTM A Pd G3] (21 mg, 0.029 mmol) were combined as solids in a microwave vial. The vial was sealed. The atmosphere was evacuated and replaced with nitrogen; this process was performed three times. Degassed dioxane (2.9 mL) and potassium phosphate solution (2.0 M in water, 860 μL, 1.7 mmol) were added, and the reaction mixture was heated at 100° C. for 1.5 h in the microwave. The reaction mixture was directly concentrated, and the crude residue was purified by column chromatography (20→100% EtOAc with 5% Et₃N/hexanes) to provide the desired product (450 mg, 0.51 mmol, 90% yield). LC/MS (ESI) m/z: [M+H]⁺ calcd for C₄₇H₆₂F₃N₆O₅Si 875.4; found 875.8.

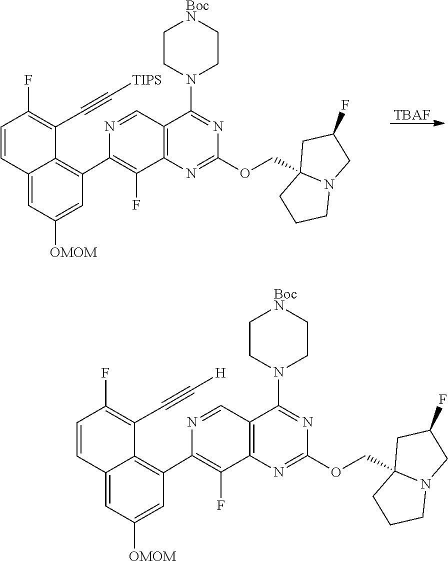

### Step 3: Preparation of tert-butyl 4-(7-(8-ethynyl-7-fluoro-3-(methoxymethoxy)naphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate

tert-Butyl 4-(8-fluoro-7-(7-fluoro-3-(methoxymethoxy)-8-((triisopropylsilyl)ethynyl)naphthalen-1-yl)-2-(((2R, 7aS)-2-fluorotetrahydro-1H-pyrrolizin-7a(5H)-yl)methoxy)pyrido[4,3-d]pyrimidin-4-yl)piperazine-1-carboxylate (500 mg, 0.57 mmol) was dissolved in THE (5.7 mL) and the solution was cooled to 0° C. TBAF solution (1.0 M in THF, 0.7 mL, 0.7 mmol) was added dropwise and the reaction mixture was stirred for 5 min. The reaction mixture was directly concentrated, and the crude residue was purified by column chromatography (50→100% EtOAc with 5% Et₃N/hexanes) to provide the desired product (quantitative yield assumed). LC/MS (ESI) m/z: [M+H]⁺ calcd for C₃₈H₄₁F₃N₆O5 719.3; found 719.6.

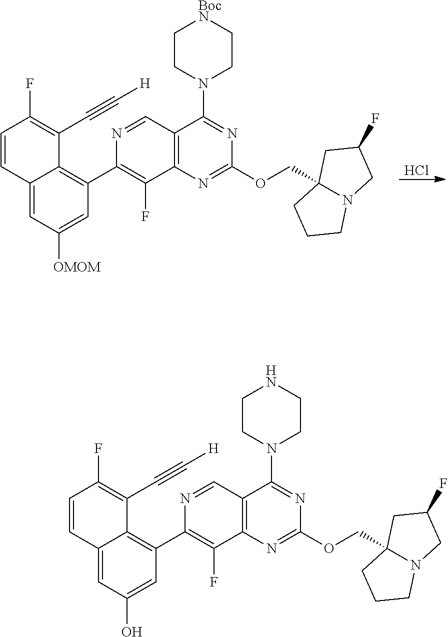

### Step 4: Preparation of 5-ethynyl-6-fluoro-4-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol

tert-Butyl 4-(7-(8-ethynyl-7-fluoro-3-(methoxymethoxy)naphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (410 mg, 0.57 mmol) was dissolved in MeCN (5.7 mL) and the solution was cooled to 0° C. HCl solution (4.0 M in dioxane, 1.4 mL, 5.7 mmol) was added dropwise, and the reaction mixture was allowed to warm to room temperature. After 2 h, sodium hydroxide solution (1.0 M, 5.7 mL, 5.7 mmol) was added, and the solution was concentrated to remove volatile organics. The resulting aqueous solution was frozen and directly lyophilized to provide the desired product along with sodium chloride. The material was of sufficient purity to be used directly in the next step, without additional purification (quantitative yield assumed). LC/MS (ESI) m/z: [M+H]* calcd for C₃₁H₃₀F₃N₆O₂ 575.2; found 575.4.

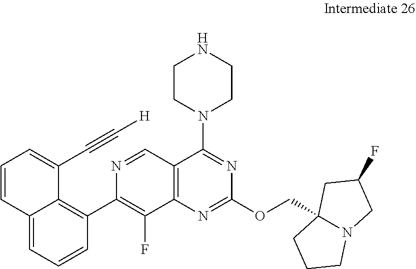 [SMILES: C#Cc1cccc2cccc(-c3ncc4c(N5CCNCC5)nc(OC[C@@]56CCCN5C[C@H](F)C6)nc4c3F)c12]

### 7-(8-ethynylnaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine

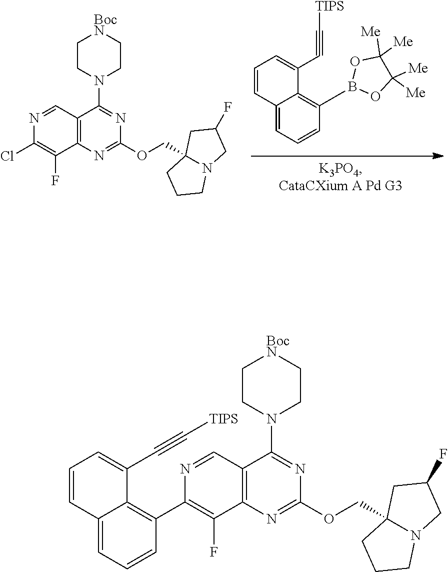

### Step 1: Preparation of tert-butyl 4-(8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)piperazine-1-carboxylate

tert-Butyl 4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (45 mg, 0.086 mmol), triisopropyl((8-(4,4,5,5-tetramethyl-1,3,2-dioxaborolan-2-yl)naphthalen-1-yl)ethynyl)silane (41 mg, 0.094 mmol) and Methanesulfonato(diadamantyl-n-butylphosphino)-2′-amino-1,1′-biphenyl-2-yl)palladium(II) dichloromethane adduct, [cataCXium© A Palladacycle Gen. 3] (3.1 mg, 4.3 μmol) were combined as solids in a microwave vial. The vial was sealed. The atmosphere was evacuated and replaced with nitrogen. This process was performed three times. Degassed dioxane (900 μL) and potassium phosphate solution (2.0 M in water, 130 μL, 0.26 mmol) were added and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (57 mg, 0.072 mmol, 83% yield). LC/MS (ESI) m/z: [M+H]⁺ calcd for C₄₅H₅₉F₂N₆O₃ Si 797.4; found 797.6; ¹H NMR (500 MHz, CDCl₃) δ 9.05 (s, 1H), 7.96 (dd, J=7.7, 1.5 Hz, 1H), 7.92 (d, J=7.9 Hz, 1H), 7.81 (d, J=6.9 Hz, 1H), 7.60-7.52 (m, 2H), 7.46 (t, J=7.7 Hz, 1H), 5.27 (d, J=55.2 Hz, 1H), 4.33-4.22 (m, 1H), 4.20-4.10 (m, 1H), 3.92 (br d, J=3.6 Hz, 4H), 3.76-3.58 (m, 4H), 3.28-3.14 (m, 2H), 2.97 (td, J=9.1, 5.5 Hz, 1H), 2.34-2.21 (m, 2H), 2.20-2.09 (m, 2H), 2.01-1.82 (m, 3H), 1.24 (s, 9H), 0.87 (dd, J=10.2, 7.5 Hz, 18H), 0.61-0.49 (m, 3H).

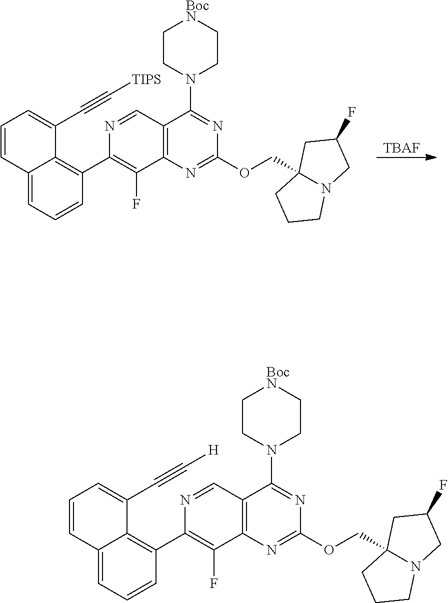

### Step 2: Preparation of tert-butyl 4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate

tert-Butyl 4-(8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)piperazine-1-carboxylate (55 mg, 0.069 mmol) and the solution was cooled to 0° C. TBAF solution (1.0 M in THF, 90 μL, 0.09 mmol) was added dropwise and the reaction mixture was warmed to room temperature. After 5 min, the reaction mixture was concentrated, and the crude residue was purified by column chromatography (50→100% EtOAc with 5% Et₃N/hexanes) to provide the desired product (42 mg, 0.066 mmol, 95% yield). LC/MS (ESI) m/z: [M+H]⁺ calcd for C₃₆H₃₉F₂N₆O₃ 641.3; found 641.3.

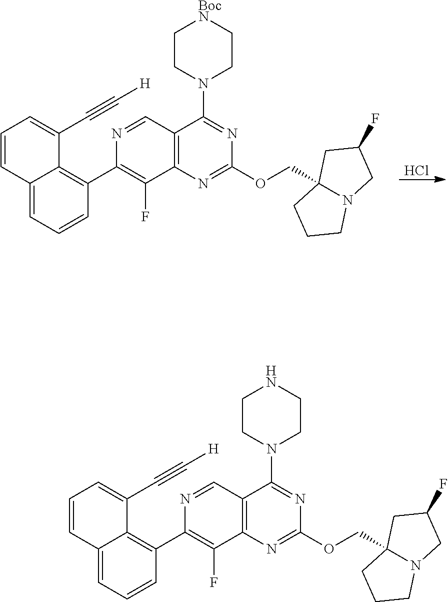 [SMILES: C#Cc1cccc2cccc(-c3ncc4c(N5CCN(C(=O)OC(C)(C)C)CC5)nc(OC[C@@]56CCCN5C[C@H](F)C6)nc4c3F)c12 ; Cl ; C#Cc1cccc2cccc(-c3ncc4c(N5CCNCC5)nc(OC[C@@]56CCCN5C[C@H](F)C6)nc4c3F)c12]

### Step 3: Preparation of 7-(8-ethynylnaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine

tert-Butyl 4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (40 mg, 0.062 mmol) was dissolved in MeCN (600 μL) and the solution was cooled to 0° C. HCl solution (4.0 M in dioxane, 160 μl, 0.62 mmol) was added dropwise. After 2 h, the reaction mixture was quenched by addition of NaOH solution (1.0 M, 0.62 mL, 0.62 mmol) and the reaction mixture was concentrated. The residue was azeotroped from toluene and used directly in the next step, without additional purification (quantitative yield assumed). LC/MS (ESI) m/z: [M+H]⁺ calcd for C₃₁H₃₁F₂N₆O 541.2; found 541.3.

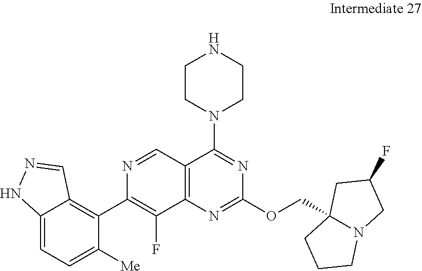 [SMILES: Cc1ccc2[nH]ncc2c1-c1ncc2c(N3CCNCC3)nc(OC[C@@]34CCCN3C[C@H](F)C4)nc2c1F]

### 8-fluoro-2-(((2R, 7aS)-2-fluorotetrahydro-1H-pyrrolizin-7a(5H)-yl)methoxy)-7-(5-methyl-1H-indazol-4-yl)-4-(piperazin-1-yl)pyrido[4,3-d]pyrimidine

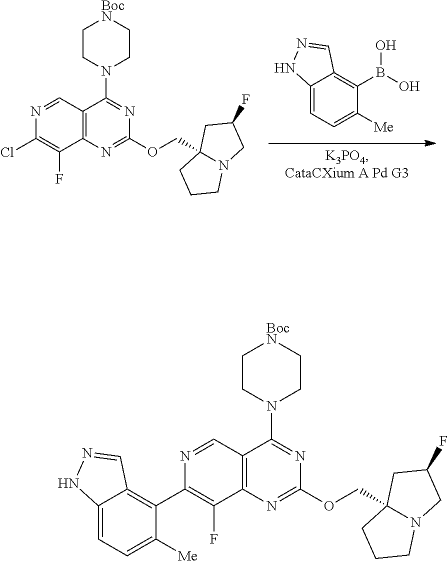 [SMILES: CC(C)(C)OC(=O)N1CCN(c2nc(OC[C@@]34CCCN3C[C@H](F)C4)nc3c(F)c(Cl)ncc23)CC1 ; Cc1ccc2[nH]ncc2c1B(O)O ; Cc1ccc2[nH]ncc2c1-c1ncc2c(N3CCN(C(=O)OC(C)(C)C)CC3)nc(OC[C@@]34CCCN3C[C@H](F)C4)nc2c1F]

### Step 1: Preparation of tert-butyl 4-(8-fluoro-2-(((2R, 7aS)-2-fluorotetrahydro-1H-pyrrolizin-7a(5H)-yl)methoxy)-7-(5-methyl-1H-indazol-4-yl)pyrido[4,3-d]pyrimidin-4-yl)piperazine-1-carboxylate

tert-Butyl 4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (50 mg, 0.095 mmol), (5-methyl-1H-indazol-4-yl)boronic acid (16.76 mg, 0.095 mmol), and methanesulfonato(diadamantyl-n-butylphosphino)-2′-amino-1,1′-biphenyl-2-yl)palladium(II) dichloromethane adduct, [cataCXium© A Palladacycle Gen. 3] (6.9 mg, 9.5 μmol) were combined as solids in a microwave vial. The vial was sealed. The atmosphere was evacuated and replaced with nitrogen. This process was performed three times. Degassed dioxane (950 μL) and potassium phosphate solution (2.0 M in water, 190 μL, 0.26 mmol) were added and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (9 mg, 0.014 mmol, 15% yield). LC/MS (ESI) m/z: [M+H]* calcd for C₃₂H₃₉F₂N₈O₃ 621.3; found 621.3.

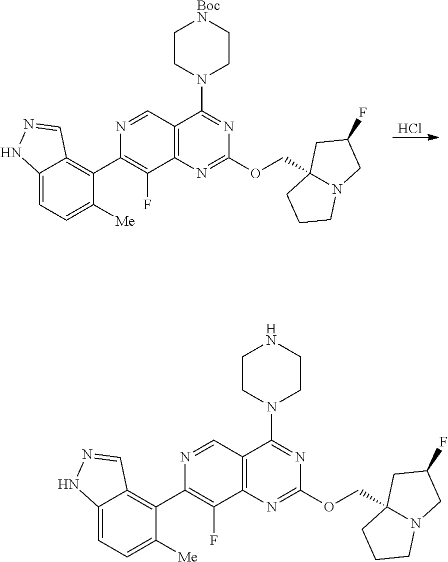 [SMILES: Cc1ccc2[nH]ncc2c1-c1ncc2c(N3CCN(C(=O)OC(C)(C)C)CC3)nc(OC[C@@]34CCCN3C[C@H](F)C4)nc2c1F ; Cl ; Cc1ccc2[nH]ncc2c1-c1ncc2c(N3CCNCC3)nc(OC[C@@]34CCCN3C[C@H](F)C4)nc2c1F]

### Step 2: Preparation of 8-fluoro-2-(((2R, 7aS)-2-fluorotetrahydro-1H-pyrrolizin-7a(5H)-yl)methoxy)-7-(5-methyl-1H-indazol-4-yl)-4-(piperazin-1-yl)pyrido[4,3-d]pyrimidine

tert-Butyl 4-(8-fluoro-2-(((2R, 7aS)-2-fluorotetrahydro-1H-pyrrolizin-7a(5H)-yl)methoxy)-7-(5-methyl-1H-indazol-4-yl)pyrido[4,3-d]pyrimidin-4-yl)piperazine-1-carboxylate (9 mg, 0.014 mmol) was dissolved in MeCN (750 μL) and the solution was cooled to 0° C. HCl solution (4.0 M in dioxane, 36 μl, 0.15 mmol) was added dropwise. After 2 h, the reaction mixture was concentrated and, the crude material (as the HCl salt) was used directly in the next step, without additional purification (quantitative yield assumed). LC/MS (ESI) m/z: [M+H]⁺ calcd for C₂₇H₃₁F₂N₈O 521.3; found 521.2.

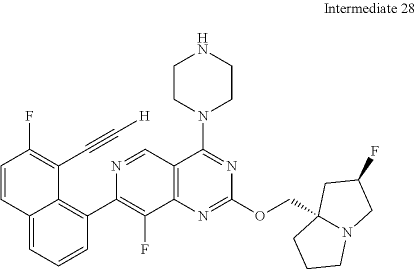 [SMILES: C#Cc1c(F)ccc2cccc(-c3ncc4c(N5CCNCC5)nc(OC[C@@]56CCCN5C[C@H](F)C6)nc4c3F)c12]

### 7-(8-ethynyl-7-fluoronaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine

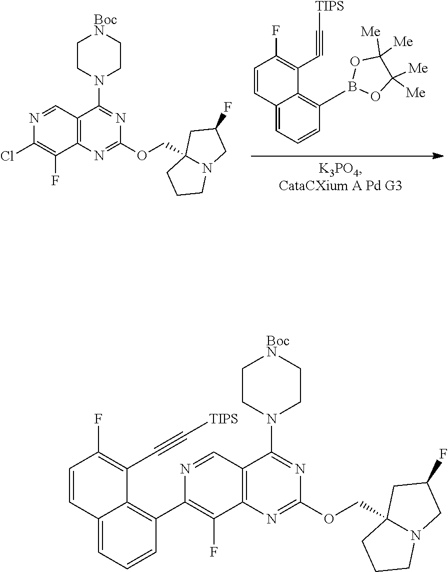

### Step 1: Preparation of tert-butyl 4-(8-fluoro-7-(7-fluoro-8-((triisopropylsilyl)ethynyl)naphthalen-1-yl)-2-(((2R, 7aS)-2-fluorotetrahydro-1H-pyrrolizin-7a(5H)-yl)methoxy)pyrido[4,3-d]pyrimidin-4-yl)piperazine-1-carboxylate

tert-Butyl 4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (171 mg, 0.33 mmol), ((2-fluoro-8-(4,4,5,5-tetramethyl-1,3,2-dioxaborolan-2-yl)naphthalen-1-yl)ethynyl)triisopropylsilane (98 mg, 0.22 mmol), and methanesulfonato(diadamantyl-n-butylphosphino)-2′-amino-1,1′-biphenyl-2-yl)palladium(II) dichloromethane adduct, [cataCXium© A Palladacycle Gen. 3] (15.8 mg, 22 μmol) were combined as solids in a microwave vial. The vial was sealed. The atmosphere was evacuated and replaced with nitrogen. This process was performed three times. Degassed dioxane (2.2 mL) and potassium phosphate solution (2.0 M in water, 430 μL, 0.65 mmol) were added and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (43 mg, 0.053 mmol, 24% yield). LC/MS (ESI) m/z: [M+H]⁺ calcd for C₄₅H₅₈F₃N₆O₃Si 815.4; found 815.9.

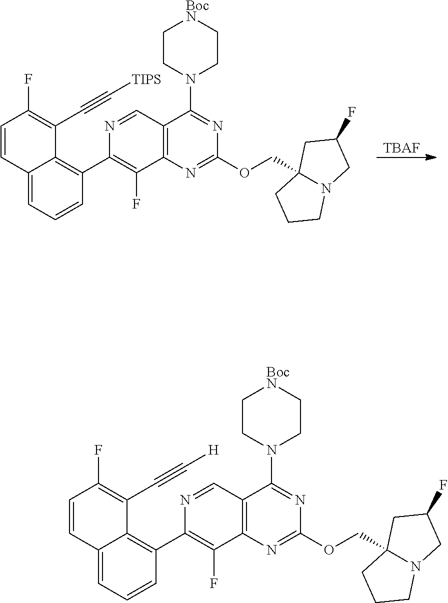

### Step 2: Preparation of tert-butyl 4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate

tert-Butyl 4-(8-fluoro-7-(7-fluoro-8-((triisopropylsilyl)ethynyl)naphthalen-1-yl)-2-(((2R, 7aS)-2-fluorotetrahydro-1H-pyrrolizin-7a(5H)-yl)methoxy)pyrido[4,3-d]pyrimidin-4-yl)piperazine-1-carboxylate (39 mg, 0.048 mmol) was dissolved in THE (1.0 mL) and the solution was cooled to 0° C. TBAF solution (1.0 M in THF, 57 μL, 0.057 mmol) was added dropwise and the reaction mixture was warmed to room temperature. After 5 min, the reaction mixture was concentrated, and the crude residue was purified by column chromatography (50→100% EtOAc with 5% Et₃N/hexanes) to provide the desired product (26 mg, 0.039 mmol, 82% yield). LC/MS (ESI) m/z: [M+H]⁺ calcd for C₃₆H₃₈F₃N₆O₃ 659.3; found 659.8.

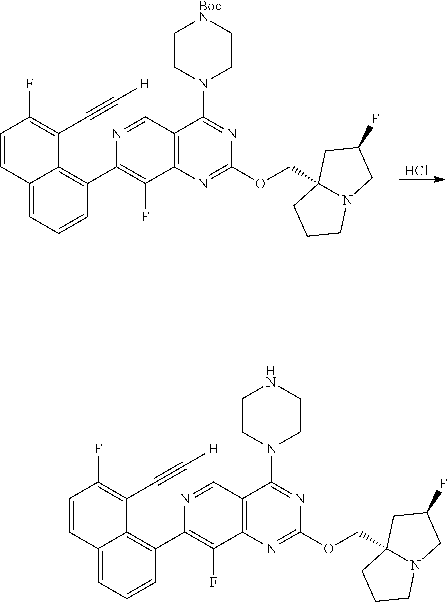 [SMILES: C#Cc1c(F)ccc2cccc(-c3ncc4c(N5CCN(C(=O)OC(C)(C)C)CC5)nc(OC[C@@]56CCCN5C[C@H](F)C6)nc4c3F)c12 ; Cl ; C#Cc1c(F)ccc2cccc(-c3ncc4c(N5CCNCC5)nc(OC[C@@]56CCCN5C[C@H](F)C6)nc4c3F)c12]

### Step 3: Preparation of 7-(8-ethynyl-7-fluoronaphthalen-1-yl)-8-fluoro-2-(((2R, 7aS)-2-fluorotetrahydro-H-pyrrolizin-7a(5H)-yl)methoxy)-4-(piperazin-1-yl)pyrido[4,3-d]pyrimidine

tert-Butyl 4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (27 mg, 0.04 mmol) was dissolved in MeCN (500 μL) and the solution was cooled to 0° C. HCl solution (4.0 M in dioxane, 100 μl, 0.41 mmol) was added dropwise. After 2 h, 1-methylimidazole (0.039 mL, 0.49 mmol) was added, and the solution was concentrated. The crude residue was used directly in the next step, without additional purification. LC/MS (ESI) m/z: [M+H]⁺ calcd for C₃₁H₃₀F₃N₆O 559.2; found 559.4.

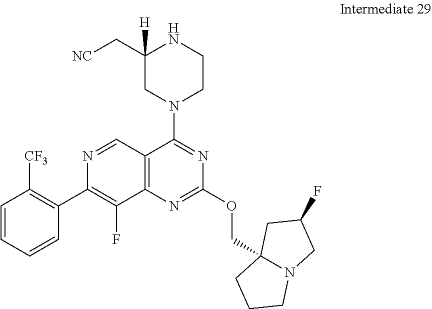 [SMILES: N#CC[C@H]1CN(c2nc(OC[C@@]34CCCN3C[C@H](F)C4)nc3c(F)c(-c4ccccc4C(F)(F)F)ncc23)CCN1]

### 2-((S)-4-(8-fluoro-2-(((2R, 7aS)-2-fluorotetrahydro-1H-pyrrolizin-7a(5H)-yl)methoxy)-7-(2-(trifluoromethyl)phenyl)pyrido[4,3-d]pyrimidin-4-yl)piperazin-2-yl)acetonitrile

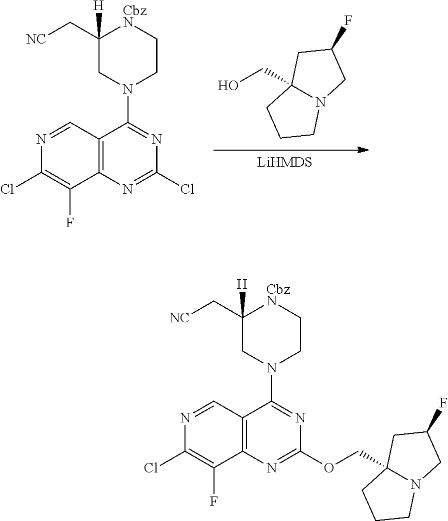

### Step 1: Preparation of benzyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate

((2R, 7aS)-2-Fluorotetrahydro-1H-pyrrolizin-7a(5H)-yl)methanol, HCl salt (680 mg, 3.5 mmol) was suspended in THE (16 mL). The mixture was cooled to 0° C., and LiHMDS solution (1.0 M in THF, 7.3 mL, 7.3 mmol) was added dropwise. Once all solid had gone into solution (5 min), benzyl (S)-2-(cyanomethyl)-4-(2,7-dichloro-8-fluoropyrido[4,3-d]pyrimidin-4-yl)piperazine-1-carboxylate (1.5 g, 3.2 mmol) was added as a solution in THE (10 mL). The reaction mixture was allowed to warm to room temperature before being gently warmed to 35° C. for 4 h. The reaction mixture was partially concentrated, and the crude residue was purified by column chromatography (50→100% EtOAc with 5% Et₃N/hexanes) to provide the desired product (1.6 g, 2.6 mmol, 83% yield) as a white foam. LC/MS (ESI) m/z: [M+H]⁺ calcd for C₂₉H₃₁C₁F₂N₇O₃ 598.2; found 598.2; ¹H NMR (500 MHz, CDCl₃) δ 8.78 (s, 1H), 7.37 (d, J=1.4 Hz, 5H), 5.26 (d, J=55.8 Hz, 1H), 5.20-5.17 (m, 2H), 4.69-4.61 (m, 1H), 4.47-4.38 (m, 1H), 4.32-4.24 (m, 2H), 4.22-4.13 (m, 2H), 3.97-3.82 (m, 1H), 3.74-3.63 (m, 1H), 3.61-3.44 (m, 1H), 3.30-3.20 (m, 2H), 3.17-3.13 (m, 1H), 3.01-2.93 (m, 1H), 2.91-2.77 (m, 1H), 2.72-2.64 (m, 1H), 2.29-2.06 (m, 3H), 1.98-1.80 (m, 3H).

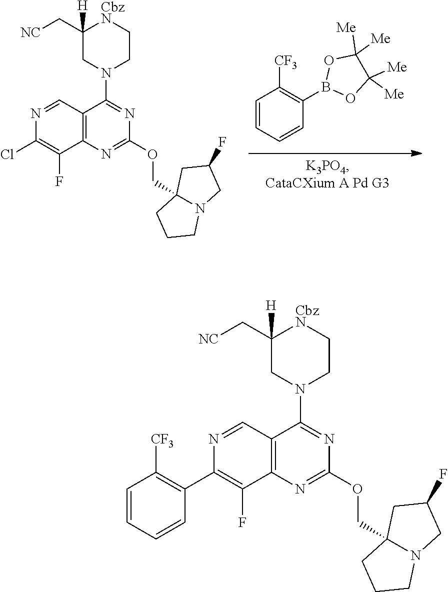

### Step 2: Preparation of benzyl (S)-2-(cyanomethyl)-4-(8-fluoro-2-(((2R, 7aS)-2-fluorotetrahydro-1H-pyrrolizin-7a(5H)-yl)methoxy)-7-(2-(trifluoromethyl)phenyl)pyrido[4,3-d]pyrimidin-4-yl)piperazine-1-carboxylate

Benzyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (50 mg, 0.084 mmol), 4,4,5,5-tetramethyl-2-(2-(trifluoromethyl)phenyl)-1,3,2-dioxaborolane (45.5 mg, 0.167 mmol), and methanesulfonato(diadamantyl-n-butylphosphino)-2′-amino-1,1′-biphenyl-2-yl)palladium(II) dichloromethane adduct, [cataCXium© A Palladacycle Gen. 3] (3.0 mg, 4 μmol) were combined as solids in a microwave vial. The vial was sealed. The atmosphere was evacuated and replaced with nitrogen. This process was performed three times. Degassed dioxane (0.8 mL) and potassium phosphate solution (2.0 M in water, 170 μL, 0.33 mmol) were added and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (58 mg, 0.08 mmol, 98% yield). LC/MS (ESI) m/z: [M+H]⁺ calcd for C₃₆H₃₄F₅N₇O₃ 708.3; found 708.3; ¹H NMR (500 MHz, CDCl₃) δ 9.03 (s, 1H), 7.83 (d, J=7.6 Hz, 1H), 7.69-7.64 (m, 1H), 7.63-7.58 (m, 1H), 7.50-7.45 (m, 1H), 7.42-7.33 (m, 5H), 5.27 (d, J=53.9 Hz, 1H), 5.21 (s, 2H), 4.74-4.66 (m, 1H), 4.53-4.43 (m, 1H), 4.41-4.34 (m, 1H), 4.30 (d, J=10.4 Hz, 1H), 4.21 (d, J=10.4 Hz, 1H), 4.25-4.16 (m, 1H), 3.94-3.79 (m, 1H), 3.75-3.65 (m, 1H), 3.63-3.46 (m, 1H), 3.28-3.18 (m, 2H), 3.01-2.93 (m, 1H), 2.93-2.79 (m, 1H), 2.78-2.69 (m, 1H), 2.31-2.09 (m, 3H), 2.00-1.81 (m, 3H).

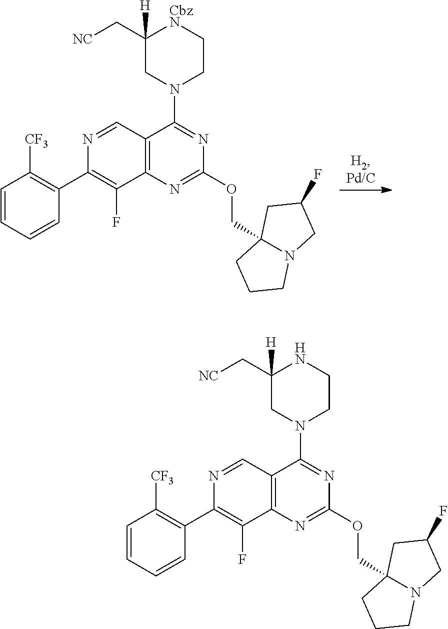 [SMILES: N#CC[C@H]1CN(c2nc(OC[C@@]34CCCN3C[C@H](F)C4)nc3c(F)c(-c4ccccc4C(F)(F)F)ncc23)CCN1C(=O)OCc1ccccc1 ; N#CC[C@H]1CN(c2nc(OC[C@@]34CCCN3C[C@H](F)C4)nc3c(F)c(-c4ccccc4C(F)(F)F)ncc23)CCN1]

### Step 3: Preparation of 2-((S)-4-(8-fluoro-2-(((2R, 7aS)-2-fluorotetrahydro-1H-pyrrolizin-7a(5H)-yl)methoxy)-7-(2-(trifluoromethyl)phenyl)pyrido[4,3-d]pyrimidin-4-yl)piperazin-2-yl)acetonitrile

Benzyl (S)-2-(cyanomethyl)-4-(8-fluoro-2-(((2R, 7aS)-2-fluorotetrahydro-1H-pyrrolizin-7a(5H)-yl)methoxy)-7-(2-(trifluoromethyl)phenyl)pyrido[4,3-d]pyrimidin-4-yl)piperazine-1-carboxylate (58 mg, 0.08 mmol) was dissolved in MeOH (0.8 mL) and palladium on carbon (10 wt. %, 18 mg, 0.017 mmol) was added. Hydrogen gas was bubbled through the solution for 5 min then the reaction mixture was kept under an atmosphere of hydrogen (balloon) for 1.5 h. The reaction mixture was filtered through a plug of diatomaceous earth (Celite®) and was concentrated. The crude residue was used directly in the next step, without additional purification (quantitative yield assumed). LC/MS (ESI) m/z: [M+H]⁺ calcd for C₂₈H₂₉F₅N₇O 574.3; found 574.2.

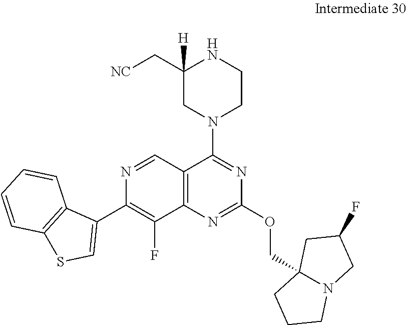 [SMILES: N#CC[C@H]1CN(c2nc(OC[C@@]34CCCN3C[C@H](F)C4)nc3c(F)c(-c4csc5ccccc45)ncc23)CCN1]

### 2-((S)-4-(7-(benzo[b]thiophen-3-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

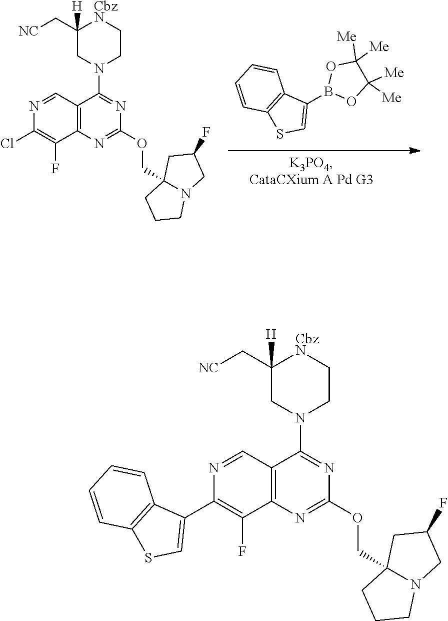 [SMILES: N#CC[C@H]1CN(c2nc(OC[C@@]34CCCN3C[C@H](F)C4)nc3c(F)c(Cl)ncc23)CCN1C(=O)OCc1ccccc1 ; CC1(C)OB(c2csc3ccccc23)OC1(C)C ; N#CC[C@H]1CN(c2nc(OC[C@@]34CCCN3C[C@H](F)C4)nc3c(F)c(-c4csc5ccccc45)ncc23)CCN1C(=O)OCc1ccccc1]

### Step 1: Preparation of benzyl (S)-4-(7-(benzo[b]thiophen-3-yl)-8-fluoro-2-(((2R, 7aS)-2-fluorotetrahydro-H-pyrrolizin-7a(5H)-yl)methoxy)pyrido[4,3-d]pyrimidin-4-yl)-2-(cyanomethyl)piperazine-1-carboxylate

Benzyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (50 mg, 0.084 mmol), 2-(benzo[b]thiophen-3-yl)-4,4,5,5-tetramethyl-1,3,2-dioxaborolane (43.5 mg, 0.167 mmol), and methanesulfonato(diadamantyl-n-butylphosphino)-2′-amino-1,1′-biphenyl-2-yl)palladium(II) dichloromethane adduct, [cataCXium© A Palladacycle Gen. 3] (3.0 mg, 4 μmol) were combined as solids in a microwave vial. The vial was sealed. The atmosphere was evacuated and replaced with nitrogen. This process was performed three times. Degassed dioxane (0.8 mL) and potassium phosphate solution (2.0 M in water, 170 μL, 0.33 mmol) were added and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (57 mg, 0.08 mmol, 98% yield). LC/MS (ESI) m/z: [M+H]⁺ calcd for C₃₇H₃₆F₂N₇O₃S 696.2; found 696.2; ¹H NMR (500 MHz, CDCl₃) δ 9.13 (s, 1H), 8.48 (d, J=7.6 Hz, 1H), 8.12 (d, J=1.3 Hz, 1H), 7.95-7.92 (m, 1H), 7.47-7.35 (m, 7H), 5.28 (d, J=53.6 Hz, 1H), 5.22-5.19 (m, 2H), 4.74-4.67 (m, 1H), 4.49 (br d, J=12.6 Hz, 1H), 4.40-4.39 (m, 1H), 4.39 (br d, J=11.9 Hz, 1H), 4.32 (d, J=10.4 Hz, 1H), 4.27-4.18 (m, 1H), 4.23 (d, J=10.4 Hz, 1H), 3.94-3.82 (m, 1H), 3.74-3.66 (m, 1H), 3.64-3.47 (m, 1H), 3.31-3.20 (m, 2H), 3.04-2.94 (m, 1H), 2.94-2.80 (m, 1H), 2.73 (dd, J=16.7, 5.6 Hz, 1H), 2.29-2.11 (m, 2H), 2.01-1.83 (m, 3H).

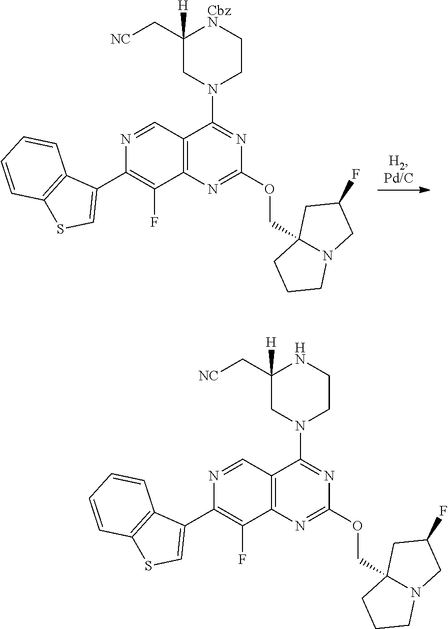 [SMILES: N#CC[C@H]1CN(c2nc(OC[C@@]34CCCN3C[C@H](F)C4)nc3c(F)c(-c4csc5ccccc45)ncc23)CCN1C(=O)OCc1ccccc1 ; N#CC[C@H]1CN(c2nc(OC[C@@]34CCCN3C[C@H](F)C4)nc3c(F)c(-c4csc5ccccc45)ncc23)CCN1]

### Step 2: Preparation of 2-((S)-4-(7-(benzo[b]thiophen-3-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

Benzyl (S)-4-(7-(benzo[b]thiophen-3-yl)-8-fluoro-2-(((2R, 7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (57 mg, 0.082 mmol) was dissolved in MeOH (0.8 mL) and palladium on carbon (10 wt. %, 17 mg, 0.016 mmol) was added. Hydrogen gas was bubbled through the solution for 5 min then the reaction mixture was kept under an atmosphere of hydrogen (balloon) for 1.5 h. The reaction mixture was filtered through a plug of diatomaceous earth (Celite©) and was concentrated. The crude residue was used directly in the next step, without additional purification (quantitative yield assumed). LC/MS (ESI) m/z: [M+H]⁺ calcd for C₂₉H₃₀F₂N₇OS 562.2; found 562.3.

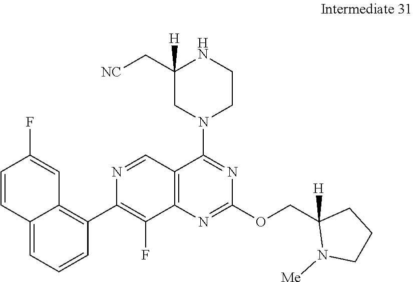 [SMILES: CN1CCC[C@H]1COc1nc(N2CCN[C@@H](CC#N)C2)c2cnc(-c3cccc4ccc(F)cc34)c(F)c2n1]

### 2-((S)-4-(8-fluoro-7-(7-fluoronaphthalen-1-yl)-2-(((S)-1-methylpyrrolidin-2-yl)methoxy)pyrido[4,3-d]pyrimidin-4-yl)piperazin-2-yl)acetonitrile

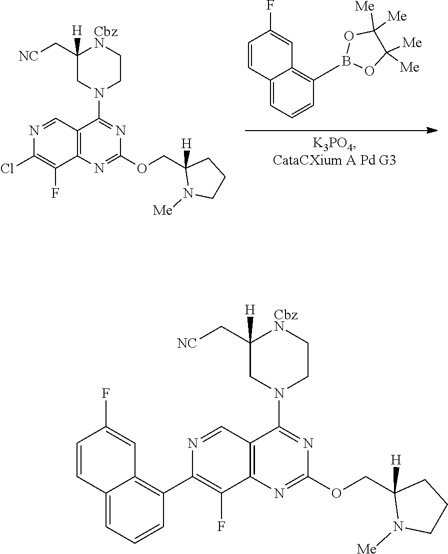 [SMILES: CN1CCC[C@H]1COc1nc(N2CCN(C(=O)OCc3ccccc3)[C@@H](CC#N)C2)c2cnc(Cl)c(F)c2n1 ; CC1(C)OB(c2cccc3ccc(F)cc23)OC1(C)C ; CN1CCC[C@H]1COc1nc(N2CCN(C(=O)OCc3ccccc3)[C@@H](CC#N)C2)c2cnc(-c3cccc4ccc(F)cc34)c(F)c2n1]

### Step 1: Preparation of benzyl (S)-2-(cyanomethyl)-4-(8-fluoro-7-(7-fluoronaphthalen-1-yl)-2-(((S)-1-methylpyrrolidin-2-yl)methoxy)pyrido[4,3-d]pyrimidin-4-yl)piperazine-1-carboxylate

Benzyl (S)-4-(7-chloro-8-fluoro-2-(((S)-1-methylpyrrolidin-2-yl)methoxy)pyrido[4,3-d]pyrimidin-4-yl)-2-(cyanomethyl)piperazine-1-carboxylate (52 mg, 0.094 mmol), 2-(7-fluoronaphthalen-1-yl)-4,4,5,5-tetramethyl-1,3,2-dioxaborolane (33 mg, 0.12 mmol), and methanesulfonato(diadamantyl-n-butylphosphino)-2′-amino-1,1′-biphenyl-2-yl)palladium(II) dichloromethane adduct, [cataCXium A Palladacycle Gen. 3](3.4 mg, 5 μmol) were combined as solids in a microwave vial. The vial was sealed. The atmosphere was evacuated and replaced with nitrogen. This process was performed three times. Degassed dioxane (0.9 mL) and potassium phosphate solution (2.0 M in water, 140 μL, 0.28 mmol) were added and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (61 mg, 0.09 mmol, 98% yield). LC/MS (ESI) m/z: [M+H]* calcd for C₃₇H₃₆F₂N₇O₃ 664.3; found 664.5; ¹H NMR (500 MHz, CDCl₃) δ 9.19 (s, 1H), 8.02-7.96 (m, 1H), 7.96-7.89 (m, 1H), 7.74-7.71 (m, 1H), 7.62-7.54 (m, 1H), 7.49-7.42 (m, 1H), 7.40-7.35 (m, 5H), 7.34-7.28 (m, 1H), 5.29-5.16 (m, 2H), 5.30 (d, J=66.2 Hz, 2H), 4.86-4.59 (m, 3H), 4.49-4.43 (m, 1H), 4.33-4.16 (m, 1H), 4.11-4.01 (m, 1H), 3.99-3.91 (m, 1H), 3.89-3.82 (m, 1H), 3.74-3.50 (m, 2H), 3.13-3.04 (m, 3H), 3.00-2.83 (m, 2H), 2.45-2.33 (m, 2H), 2.28-2.17 (m, 1H), 2.13-2.09 (m, 1H).

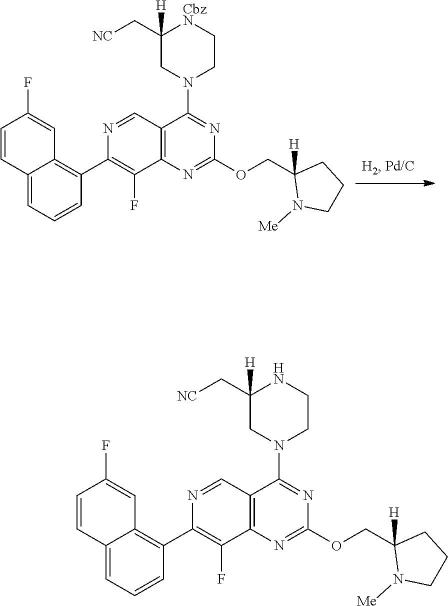 [SMILES: CN1CCC[C@H]1COc1nc(N2CCN(C(=O)OCc3ccccc3)[C@@H](CC#N)C2)c2cnc(-c3cccc4ccc(F)cc34)c(F)c2n1 ; CN1CCC[C@H]1COc1nc(N2CCN[C@@H](CC#N)C2)c2cnc(-c3cccc4ccc(F)cc34)c(F)c2n1]

### Step 2: Preparation of 2-((S)-4-(8-fluoro-7-(7-fluoronaphthalen-1-yl)-2-(((S)-1-methylpyrrolidin-2-yl)methoxy)pyrido[4,3-d]pyrimidin-4-yl)piperazin-2-yl)acetonitrile

Benzyl (S)-2-(cyanomethyl)-4-(8-fluoro-7-(7-fluoronaphthalen-1-yl)-2-(((S)-1-methylpyrrolidin-2-yl)methoxy)pyrido[4,3-d]pyrimidin-4-yl)piperazine-1-carboxylate (61 mg, 0.092 mmol) was dissolved in MeOH (2.0 mL) and palladium on carbon (10 wt. %, 20 mg, 0.018 mmol) was added. Hydrogen gas was bubbled through the solution for 5 min then the reaction mixture was kept under an atmosphere of hydrogen (balloon) for 1.5 h. The reaction mixture was filtered through a plug of diatomaceous earth (Celite©) and was concentrated. The crude residue was used directly in the next step, without additional purification (quantitative yield assumed). LC/MS (ESI) m/z: [M+H]⁺ calcd for C₂₉H₃₀F₂N₇O 530.2; found 530.2.

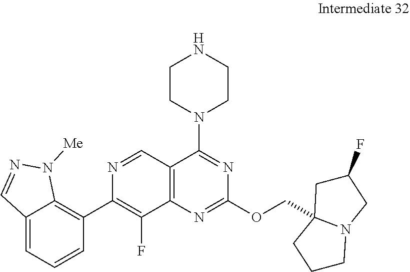

### 8-fluoro-2-(((2R, 7aS)-2-fluorotetrahydro-1H-pyrrolizin-7a(5H)-yl)methoxy)-7-(1-methyl-1H-indazol-7-yl)-4-(piperazin-1-yl)pyrido[4,3-d]pyrimidine

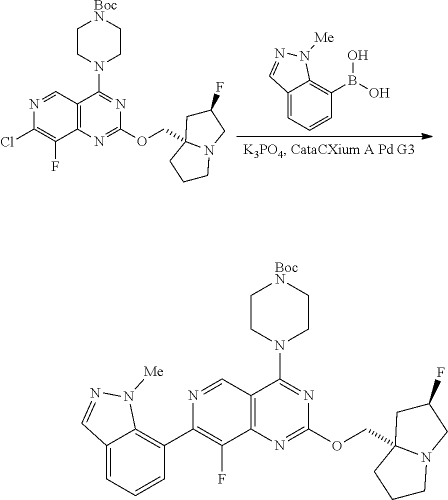

### Step 1: Preparation of tert-butyl 4-(8-fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)piperazine-1-carboxylate

tert-Butyl 4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (75 mg, 0.13 mmol), (1-methyl-1H-indazol-7-yl)boronic acid (38 mg, 0.21 mmol), and methanesulfonato(diadamantyl-n-butylphosphino)-2′-amino-1,1′-biphenyl-2-yl)palladium(II) dichloromethane adduct, [cataCXium© A Palladacycle Gen. 3] (10.4 mg, 14 μmol) were combined as solids in a microwave vial. The vial was sealed. The atmosphere was evacuated and replaced with nitrogen. This process was performed three times. Degassed dioxane (1.4 mL) and potassium phosphate solution (2.0 M in water, 290 μL, 0.33 mmol) were added and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (29 mg, 0.05 mmol, 33% yield). LC/MS (ESI) m/z: [M+H]* calcd for C₃₂H₃₉F₂N₈O₃ 621.3; found 621.4; ¹H NMR (500 MHz, CDCl₃) δ 9.10 (s, 1H), 8.07 (s, 1H), 7.85 (dd, J=8.0, 1.0 Hz, 1H), 7.47 (d, J=7.0 Hz, 1H), 7.25 (d, J=1.2 Hz, 1H), 5.43 (d, J=51.9 Hz, 1H), 4.04 (br s, 4H), 3.99-3.91 (m, 1H), 3.77 (s, 3H), 3.73-3.69 (m, 4H), 3.68-3.65 (m, 1H), 3.52-3.04 (m, 4H), 2.66-2.02 (m, 6H), 1.50 (s, 9H).

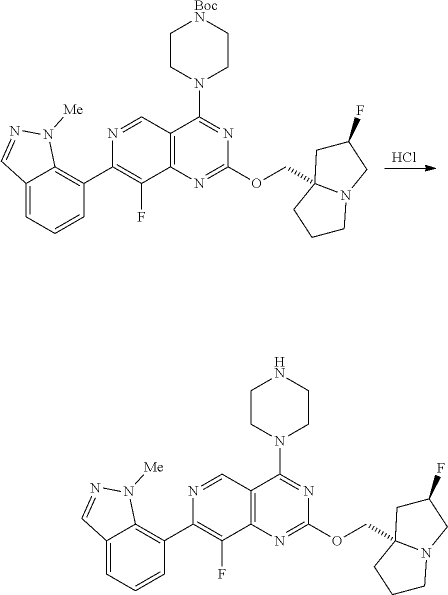

### Step 2: Preparation of 8-fluoro-2-(((2R, 7aS)-2-fluorotetrahydro-1H-pyrrolizin-7a(5H)-yl)methoxy)-7-(1-methyl-1H-indazol-7-yl)-4-(piperazin-1-yl)pyrido[4,3-d]pyrimidine

tert-Butyl 4-(8-fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)piperazine-1-carboxylate (29 mg, 0.047 mmol) was dissolved in MeCN (1.0 mL) and the solution was cooled to 0° C. HCl solution (4.0 M in dioxane, 120 μl, 0.47 mmol) was added dropwise. After 2 h, the solution was concentrated, and the crude residue was used directly in the next step, without additional purification (quantitative yield assumed). LC/MS (ESI) m/z: [M+H]⁺ calcd for C₂₇H₃₁F₂N₈O 521.3; found 521.3.

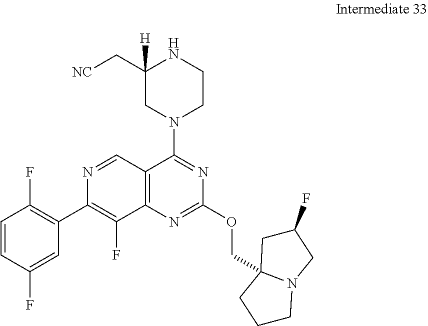

### 2-((S)-4-(7-(2,5-difluorophenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

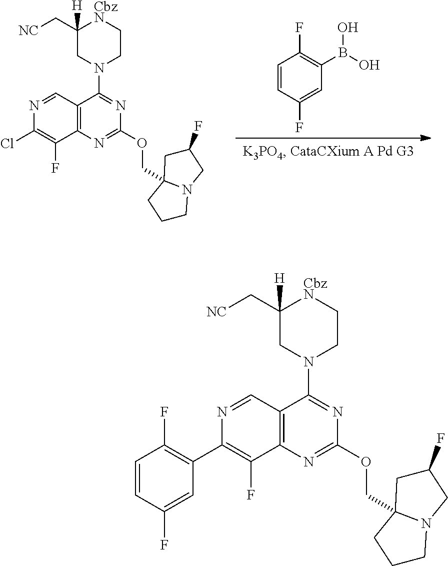

### Step 1: benzyl (S)-2-(cyanomethyl)-4-(7-(2,5-difluorophenyl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate

Benzyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (40 mg, 0.067 mmol), (2,5-difluorophenyl)boronic acid (21 mg, 0.134 mmol), and methanesulfonato(diadamantyl-n-butylphosphino)-2′-amino-1,1′-biphenyl-2-yl)palladium(II) dichloromethane adduct, [cataCXium© A Palladacycle Gen. 3] (2.4 mg, 3 μmol)were combined as solids in a microwave vial. The vial was sealed. The atmosphere was evacuated and replaced with nitrogen. This process was performed three times. Degassed dioxane (0.7 mL)and potassium phosphate solution (2.0 M in water, 100 μL, 0.20 mmol)were added and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (41 mg, 0.061 mmol, 91% yield). LC/MS (ESI) m/z: [M+H]⁺ calcd for C₃₅H₃₄F₄N₇O₃ 676.3; found 676.5; ¹H NMR (500 MHz, CDCl₃) δ 9.06 (s, 1H), 7.38 (s, 6H), 7.20-7.13 (m, 2H), 5.27 (d, J=53.8 Hz, 1H), 5.20 (s, 2H), 4.74-4.65 (m, 1H), 4.51-4.44 (m, 1H), 4.40-4.34 (m, 1H), 4.31 (d, J=10.5 Hz, 1H), 4.22 (d, J=10.5 Hz, 1H), 4.20-4.11 (m, 1H), 3.94-3.81 (m, 1H), 3.74-3.65 (m, 1H), 3.62-3.46 (m, 1H), 3.29-3.15 (m, 3H), 2.97 (td, J=9.2, 5.8 Hz, 1H), 2.93-2.77 (m, 1H), 2.71 (dd, J=16.7, 5.2 Hz, 1H), 2.27-2.08 (m, 3H), 2.00-1.82 (m, 3H).

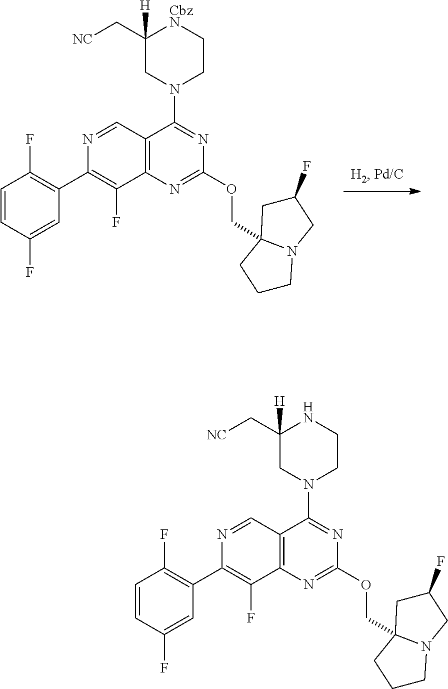

### Step 2: Preparation of 2-((S)-4-(7-(2,5-difluorophenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

Benzyl (S)-2-(cyanomethyl)-4-(7-(2,5-difluorophenyl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (40 mg, 0.059 mmol) was dissolved in MeOH (1.1 mL) and palladium on carbon (10 wt. %, 13 mg, 0.012 mmol) was added. Hydrogen gas was bubbled through the solution for 5 min then the reaction mixture was kept under an atmosphere of hydrogen (balloon) for 1.5 h. The reaction mixture was filtered through a plug of diatomaceous earth (Celite©) and was concentrated. The crude residue was used directly in the next step, without additional purification (quantitative yield assumed). LC/MS (ESI) m/z: [M+H]⁺ calcd for C₂₇H₂₈F₄N₇O 542.2; found 542.3.

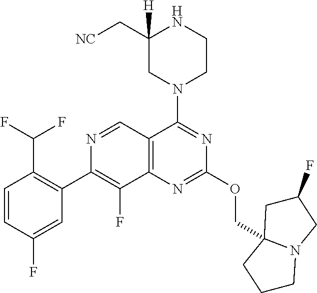

### 2-((S)-4-(7-(2-(difluoromethyl)-5-fluorophenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

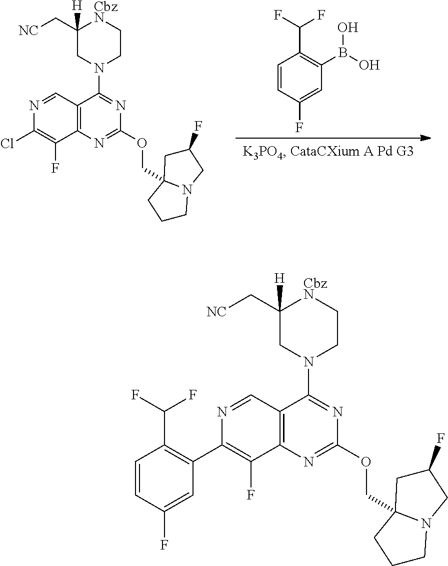

### Step 1: Preparation of benzyl (S)-2-(cyanomethyl)-4-(7-(2-(difluoromethyl)-5-fluorophenyl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate

Benzyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (40 mg, 0.067 mmol), (2-(difluoromethyl)-5-fluorophenyl)boronic acid (25 mg, 0.13 mmol and methanesulfonato(diadamantyl-n-butylphosphino)-2′-amino-1,1′-biphenyl-2-yl)palladium(II) dichloromethane adduct, [cataCXium©A Palladacycle Gen. 3](2.4 mg, 3 μmol) were combined as solids in a microwave vial. The vial was sealed. The atmosphere was evacuated and replaced with nitrogen. This process was performed three times. Degassed dioxane (0.7 mL) and potassium phosphate solution (2.0 M in water, 100 μL, 0.20 mmol) were added and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (45 mg, 0.064 mmol, 95% yield). LC/MS (ESI) m/z: [M+H]* calcd for C₃₆H₃₅F₅N₇O₃ 708.3; found 708.6; ¹H NMR (500 MHz, CDCl₃) δ 9.05 (s, 1H), 7.84 (dd, J=8.5, 5.3 Hz, 1H), 7.41-7.36 (m, 5H), 7.33-7.27 (m, 2H), 6.95 (t, J=55.3 Hz, 1H), 5.27 (d, J=54.6 Hz, 1H), 5.21-5.19 (m, 2H), 4.72-4.66 (m, 1H), 4.53-4.45 (m, 1H), 4.42-4.35 (m, 1H), 4.31 (d, J=10.5 Hz, 1H), 4.22 (d, J=10.5 Hz, 1H), 4.23-4.15 (m, 1H), 3.98-3.85 (m, 1H), 3.71 (td, J=11.6, 3.9 Hz, 1H), 3.64-3.46 (m, 1H), 3.31-3.12 (m, 3H), 3.02-2.93 (m, 1H), 2.93-2.78 (m, 1H), 2.77-2.66 (m, 1H), 2.28-2.07 (m, 3H), 1.98-1.85 (m, 3H).

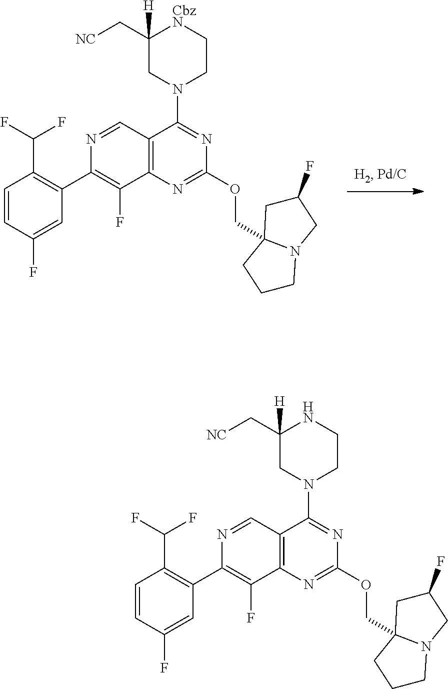

### Step 2: Preparation of 2-((S)-4-(7-(2-(difluoromethyl)-5-fluorophenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

Benzyl (S)-2-(cyanomethyl)-4-(7-(2-(difluoromethyl)-5-fluorophenyl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (40 mg, 0.057 mmol) was dissolved in MeOH (1.1 mL) and palladium on carbon (10 wt. %, 12 mg, 0.011 mmol) was added. Hydrogen gas was bubbled through the solution for 5 min then the reaction mixture was kept under an atmosphere of hydrogen (balloon) for 1.5 h. The reaction mixture was filtered through a plug of diatomaceous earth (Celite©) and was concentrated. The crude residue was used directly in the next step, without additional purification (quantitative yield assumed). LC/MS (ESI) m/z: [M+H]⁺ calcd for C₂₈H₂₉F₅N₇O 574.2; found 574.3.

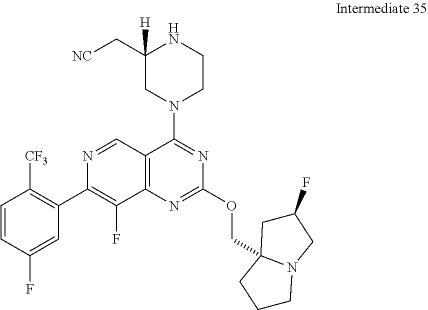

### 2-((S)-4-(8-fluoro-7-(5-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

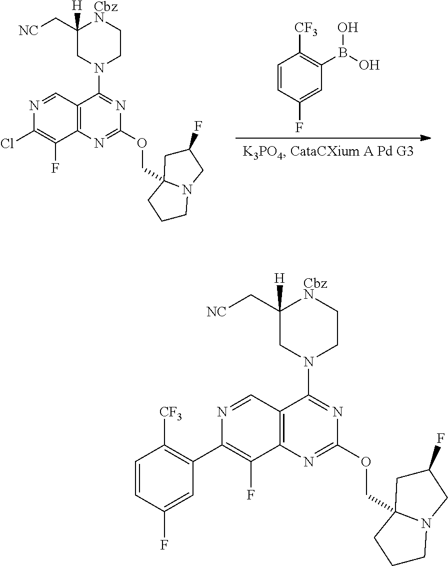

### Step 1: Preparation of benzyl (S)-2-(cyanomethyl)-4-(8-fluoro-7-(5-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazine-1-carboxylate)

Benzyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (40 mg, 0.067 mmol), (5-fluoro-2-(trifluoromethyl)phenyl)boronic acid (27.8 mg, 0.134 mmol), and methanesulfonato(diadamantyl-n-butylphosphino)-2′-amino-1,1′-biphenyl-2-yl)palladium(II) dichloromethane adduct, [cataCXium A Palladacycle Gen. 3](2.4 mg, 3 μmol) were combined as solids in a microwave vial. The vial was sealed. The atmosphere was evacuated and replaced with nitrogen. This process was performed three times. Degassed dioxane (0.7 mL) and potassium phosphate solution (2.0 M in water, 100 μL, 0.20 mmol) were added and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (45 mg, 0.062 mmol, 93% yield). LC/MS (ESI) m/z: [M+H]* calcd for C₃₆H₃₄F₆N₇O₃ 726.3; found 726.6; ¹H NMR (600 MHz, CDCl₃) δ 9.02 (s, 1H), 7.83 (dd, J=8.8, 5.2 Hz, 1H), 7.44-7.33 (m, 5H), 7.32-7.27 (m, 1H), 7.20 (dd, J=8.5, 2.4 Hz, 1H), 5.27 (d, J=54.3 Hz, 1H), 5.21 (s, 2H), 4.76-4.64 (m, 1H), 4.53-4.34 (m, 2H), 4.30 (d, J=10.4 Hz, 1H), 4.21 (d, J=10.4 Hz, 1H), 4.24-4.10 (m, 1H), 4.00-3.82 (m, 1H), 3.77-3.67 (m, 1H), 3.65-3.42 (m, 1H), 3.31-3.15 (m, 3H), 3.02-2.93 (m, 1H), 2.93-2.77 (m, 1H), 2.72 (br dd, J=16.7, 5.3 Hz, 1H), 2.29-2.08 (m, 3H), 1.97-1.83 (m, 3H).

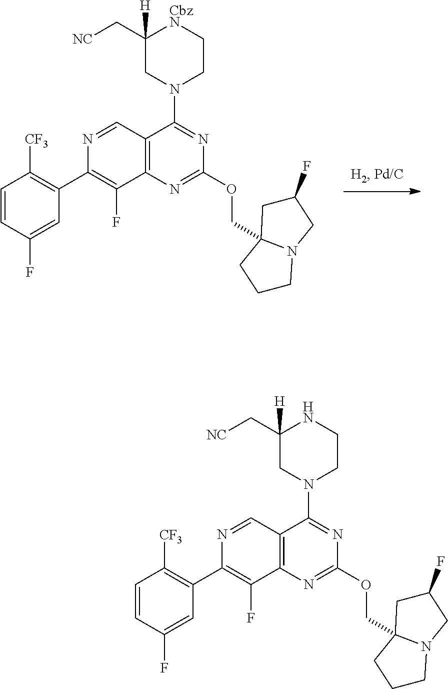

### Step 2: Preparation of 2-((S)-4-(8-fluoro-7-(5-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

Benzyl (S)-2-(cyanomethyl)-4-(8-fluoro-7-(5-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazine-1-carboxylate (40 mg, 0.055 mmol) was dissolved in MeOH (1.1 mL) and palladium on carbon (10 wt. %, 12 mg, 0.011 mmol) was added. Hydrogen gas was bubbled through the solution for 5 min then the reaction mixture was kept under an atmosphere of hydrogen (balloon) for 1.5 h. The reaction mixture was filtered through a plug of diatomaceous earth (Celite©) and was concentrated. The crude residue was used directly in the next step, without additional purification (quantitative yield assumed). LC/MS (ESI) m/z: [M+H]⁺ calcd for C₂₈H₂₈F₆N₇O 592.2; found 592.3.

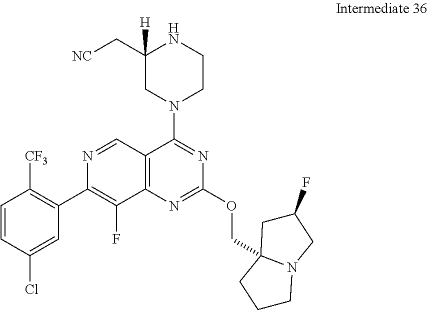

### 2-((S)-4-(8-fluoro-7-(5-chloro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

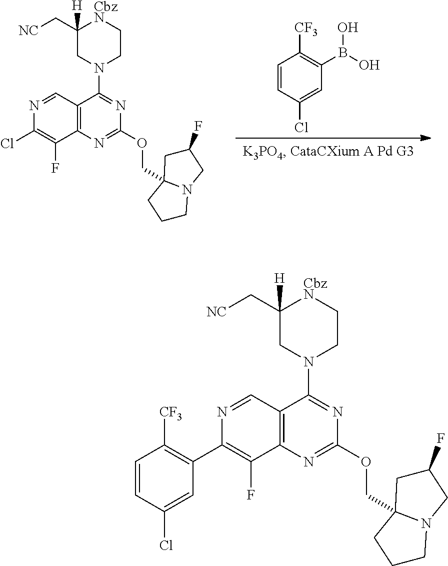

### Step 1: Preparation of benzyl (S)-2-(cyanomethyl)-4-(8-fluoro-7-(5-chloro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazine-1-carboxylate)

Benzyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (40 mg, 0.067 mmol), (5-chloro-2-(trifluoromethyl)phenyl)boronic acid (30 mg, 0.134 mmol), and methanesulfonato(diadamantyl-n-butylphosphino)-2′-amino-1,1′-biphenyl-2-yl)palladium(II) dichloromethane adduct, [cataCXium A Palladacycle Gen. 3](2.4 mg, 3 μmol) were combined as solids in a microwave vial. The vial was sealed. The atmosphere was evacuated and replaced with nitrogen. This process was performed three times. Degassed dioxane (0.7 mL) and potassium phosphate solution (2.0 M in water, 100 μL, 0.20 mmol) were added and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (24 mg, 0.03 mmol, 48% yield). LC/MS (ESI) m/z: [M+H]* calcd for C₃₆H₃₄C₁F₅N₇O₃ 742.2; found 742.1; ¹H NMR (600 MHz, CDCl₃) δ 9.02 (s, 1H), 7.76 (d, J=8.5 Hz, 1H), 7.58 (d, J=8.4 Hz, 1H), 7.49-7.47 (m, 1H), 7.42-7.34 (m, 5H), 5.27 (d, J=54.0 Hz, 1H), 5.21 (s, 2H), 4.74-4.62 (m, 1H), 4.53-4.44 (m, 1H), 4.41-4.34 (m, 1H), 4.32-4.14 (m, 3H), 3.99-3.79 (m, 1H), 3.76-3.66 (m, 1H), 3.64-3.41 (m, 1H), 3.32-3.14 (m, 3H), 3.05-2.93 (m, 1H), 2.93-2.76 (m, 1H), 2.76-2.67 (m, 1H), 2.29-2.08 (m, 3H), 1.99-1.77 (m, 3H).

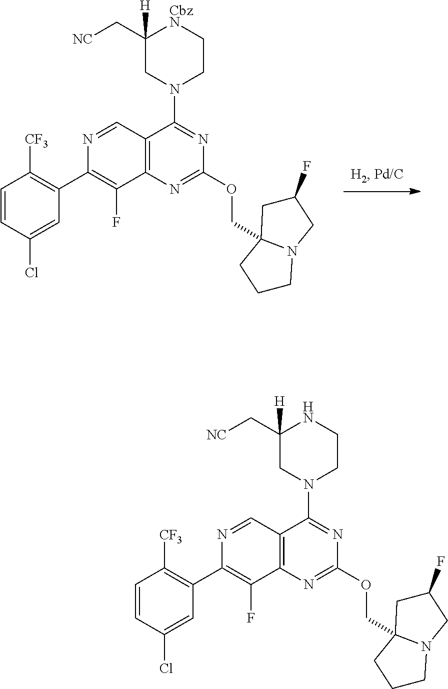

### Step 2: Preparation of 2-((S)-4-(8-fluoro-7-(5-chloro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

Benzyl (S)-2-(cyanomethyl)-4-(8-fluoro-7-(5-chloro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazine-1-carboxylate (20 mg, 0.027 mmol) was dissolved in MeOH (0.55 mL) and palladium on carbon (10 wt. %, 6 mg, 0.027 mmol) was added. Hydrogen gas was bubbled through the solution for 5 min then the reaction mixture was kept under an atmosphere of hydrogen (balloon) for 1.5 h. The reaction mixture was filtered through a plug of diatomaceous earth (Celite©) and was concentrated. The crude residue was used directly in the next step, without additional purification (quantitative yield assumed). LC/MS (ESI) m/z: [M+H]⁺ calcd for C₂₈H₂₈ClF₅N₇O 608.2; found 608.1.

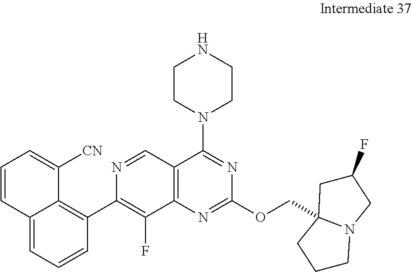

### 8-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)-1-naphthonitrile

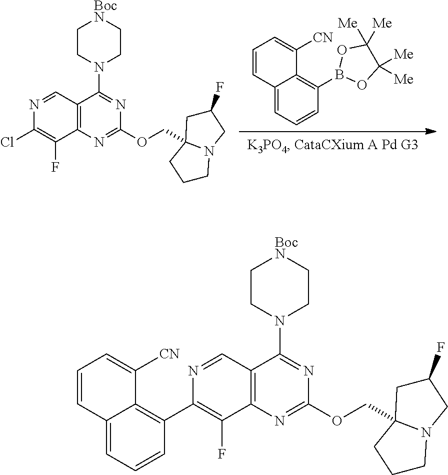

### Step 1: Preparation of tert-butyl 4-(7-(8-cyanonaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate

tert-Butyl 4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (50 mg, 0.095 mmol), 8-(4,4,5,5-tetramethyl-1,3,2-dioxaborolan-2-yl)-1-naphthonitrile (35 mg, 0.125 mmol), and methanesulfonato(diadamantyl-n-butylphosphino)-2′-amino-1,1′-biphenyl-2-yl)palladium(II) dichloromethane adduct, [cataCXium© A Palladacycle Gen. 3] (6.9 mg, 9.5 μmol) were combined as solids in a microwave vial. The vial was sealed. The atmosphere was evacuated and replaced with nitrogen. This process was performed three times. Degassed THF (0.95 mL) and potassium phosphate solution (2.0 M in water, 190 μL, 0.29 mmol) were added and the reaction mixture was heated at 65° C. in the microwave for 1.5 h. The reaction mixture was directly concentrated, and the crude residue was purified by column chromatography (20→100% EtOAc with 5% Et₃N/hexanes) to provide the desired product (14 mg, 0.022 mmol, 23% yield). LC/MS (ESI) m/z: [M+H]* calcd for C₃₅H₃₈F₂N₇O₃ 642.3; found 642.2.

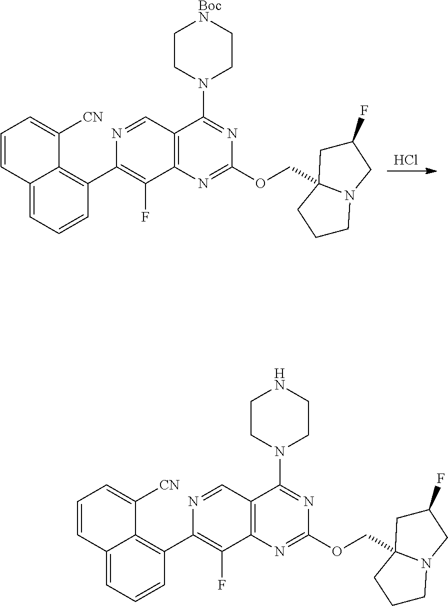

### Step 2: Preparation of 8-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)-1-naphthonitrile

tert-Butyl 4-(7-(8-cyanonaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (14.1 mg, 0.022 mmol) was dissolved in MeCN (1.0 mL) and the solution was cooled to 0° C. HCl solution (4.0 M in dioxane, 55 μl, 0.22 mmol) was added dropwise. After 2 h, the solution was concentrated, and the crude residue was used directly in the next step, without additional purification (quantitative yield assumed). LC/MS (ESI) m/z: [M+H]⁺ calcd for C₃₀H₃₀F₂N₇O 542.2; found 542.5.

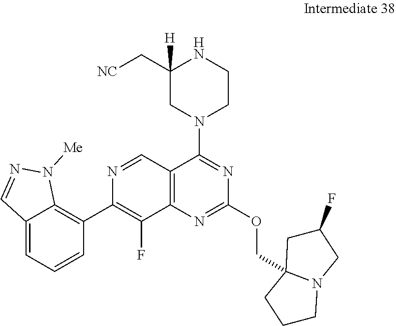

### 2-((S)-4-(8-fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)piperazin-2-yl)acetonitrile

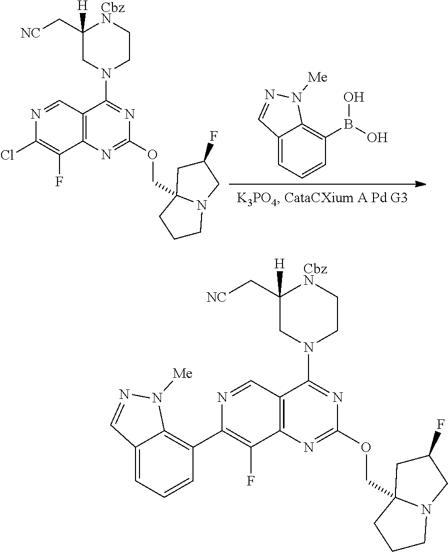

### Step 1: Preparation of benzyl (S)-2-(cyanomethyl)-4-(8-fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)piperazine-1-carboxylate

Benzyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (75 mg, 0.125 mmol), (1-methyl-1H-indazol-7-yl)boronic acid (33 mg, 0.19 mmol), and methanesulfonato(diadamantyl-n-butylphosphino)-2′-amino-1,1′-biphenyl-2-yl)palladium(II) dichloromethane adduct, [cataCXium© A Palladacycle Gen. 3] (9.1 mg, 13 μmol) were combined as solids in a microwave vial. The vial was sealed. The atmosphere was evacuated and replaced with nitrogen. This process was performed three times. Degassed dioxane (1.3 mL) and potassium phosphate solution (2.0 M in water, 250 μL, 0.20 mmol) were added and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (59 mg, 0.085 mmol, 68% yield). LC/MS (ESI) m/z: [M+H]* calcd for C₃₇H₃₈F₂N₉O₃ 694.3; found 649.6.

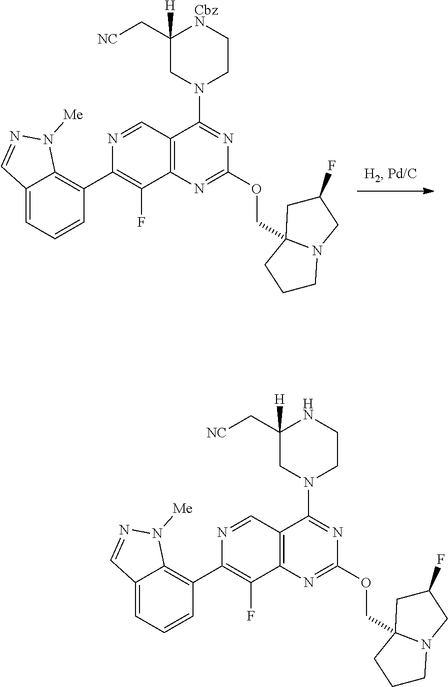

### Step 2: Preparation of 2-((S)-4-(8-fluoro-2-(((2R, 7aS)-2-fluorotetrahydro-1H-pyrrolizin-7a(5H)-yl)methoxy)-7-(I-methyl-1H-indazol-7-yl)pyrido[4,3-d]pyrimidin-4-yl)piperazin-2-yl)acetonitrile

Benzyl (S)-2-(cyanomethyl)-4-(8-fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)piperazine-1-carboxylate (59 mg, 0.085 mmol) was dissolved in MeOH (3.0 mL) and palladium on carbon (10 wt. %, 9 mg, 0.008 mmol) was added. Hydrogen gas was bubbled through the solution for 5 min then the reaction mixture was kept under an atmosphere of hydrogen (balloon) for 1.5 h. The reaction mixture was filtered through a plug of diatomaceous earth (Celite©) and was concentrated. The crude residue was used directly in the next step, without additional purification (quantitative yield assumed). LC/MS (ESI) m/z: [M+H]⁺ calcd for C₂₉H₃₂F₂N₉O 560.3; found 560.6.

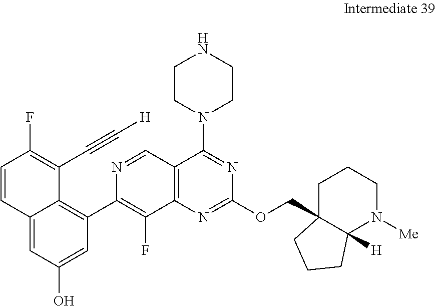 [SMILES: C#Cc1c(F)ccc2cc(O)cc(-c3ncc4c(N5CCNCC5)nc(OC[C@]56CCC[C@H]5N(C)CCC6)nc4c3F)c12]

### 5-ethynyl-6-fluoro-4-(8-fluoro-2-(((4aS,7aR)-1-methyloctahydro-4aH-cyclopenta[b]pyridin-4a-yl)methoxy)-4-(piperazin-1-yl)pyrido[4,3-d]pyrimidin-7-yl)naphthalen-2-ol

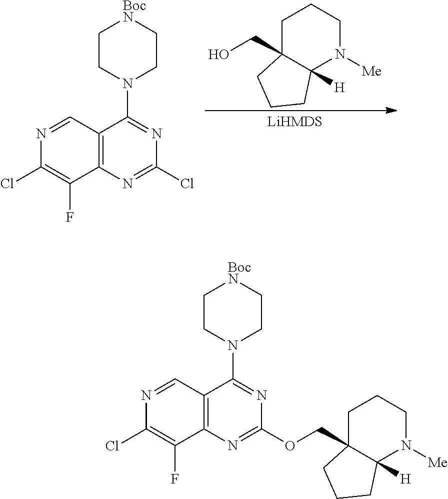 [SMILES: CC(C)(C)OC(=O)N1CCN(c2nc(Cl)nc3c(F)c(Cl)ncc23)CC1 ; CN1CCC[C@@]2(CO)CCC[C@@H]12 ; CN1CCC[C@@]2(COc3nc(N4CCN(C(=O)OC(C)(C)C)CC4)c4cnc(Cl)c(F)c4n3)CCC[C@@H]12]

### Step 1: Preparation of tert-butyl 4-(7-chloro-8-fluoro-2-(((4aS, 7aR)-1-methyloctahydro-4aH-cyclopenta[b]pyridin-4a-yl)methoxy)pyrido[4,3-d]pyrimidin-4-yl)piperazine-1-carboxylate

((4aS,7aR)-1-Methyloctahydro-4aH-cyclopenta[b]pyridin-4a-yl)methanol (prepared according to the procedure described in WO2022/192794, 126 mg, 0.75 mmol) was dissolved in THE (2.5 mL) and the solution was cooled to 0° C. LiHMDS solution (1.0 M in THF, 720 μL, 0.721 mmol) was added dropwise, and the reaction mixture was stirred for 10 min. tert-Butyl 4-(2,7-dichloro-8-fluoropyrido[4,3-d]pyrimidin-4-yl)piperazine-1-carboxylate (200 mg, 0.5 mmol) was added as a solid. The mixture was allowed to warm to room temperature, and was stirred for 16 h. The reaction mixture was partially concentrated, and the crude residue was purified by column chromatography (50→100% EtOAc with 5% Et₃N/hexanes) to provide the desired product (240 mg, 0.45 mmol, 91% yield). LC/MS (ESI) m/z: [M+H]⁺ calcd for C₂₆H₃₇C₁FN₆O₃ 535.3; found 535.6; ¹H NMR (500 MHz, CDCl₃) δ 8.74 (s, 1H), 4.48 (d, J=10.7 Hz, 1H), 4.28 (d, J=10.6 Hz, 1H), 3.98-3.90 (m, 4H), 3.69-3.63 (m, 4H), 2.71 (t, J=6.2 Hz, 1H), 2.58 (ddd, J=11.2, 7.2, 4.2 Hz, 1H), 2.32-2.29 (m, 1H), 2.27 (s, 3H), 1.89-1.60 (m, 10H), 1.50 (s, 9H).

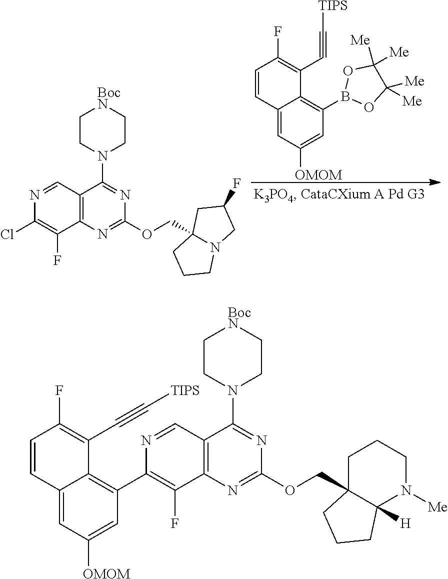

### Step 2: Preparation of tert-butyl 4-(8-fluoro-7-(7-fluoro-3-(methoxymethoxy)-8-((triisopropylsilyl)ethynyl)naphthalen-1-yl)-2-(((4aS,7aR)-1-methyloctahydro-4aH-cyclopenta[b]pyridin-4a-yl)methoxy)pyrido[4,3-d]pyrimidin-4-yl)piperazine-1-carboxylate

tert-Butyl 4-(7-chloro-8-fluoro-2-(((4aS,7aR)-1-methyloctahydro-4aH-cyclopenta[b]pyridin-4a-yl)methoxy)pyrido[4,3-d]pyrimidin-4-yl)piperazine-1-carboxylate (240 mg, 0.449 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (440 mg, 0.86 mmol), and methanesulfonato(diadamantyl-n-butylphosphino)-2′-amino-1,1′-biphenyl-2-yl)palladium(II) dichloromethane adduct [cataCXium A Pd G3] (16 mg, 0.022 mmol) were combined as solids in a microwave vial. The vial was sealed. The atmosphere was evacuated and replaced with nitrogen; this process was performed three times. Degassed dioxane (2.2 mL) and potassium phosphate solution (2.0 M in water, 670 μL, 1.3 mmol) were added, and the reaction mixture was heated at 100° C. for 1.5 h in the microwave. The reaction mixture was directly concentrated, and the crude residue was purified by column chromatography (20→100% EtOAc with 5% Et₃N/hexanes) to provide the desired product (quantitative yield assumed). LC/MS (ESI) m/z: [M+H]⁺ calcd for C₄₉H₆₇F₂N₆O₅Si 885.5; found 885.9; ¹H NMR (500 MHz, CDCl₃) δ 9.04 (d, J=1.0 Hz, 1H), 7.78 (dd, J=9.1, 5.6 Hz, 1H), 7.51 (d, J=2.6 Hz, 1H), 7.33 (d, J=2.5 Hz, 1H), 7.29 (t, J=8.8 Hz, 1H), 5.31 (d, J=7.0 Hz, 1H), 5.28 (d, J=7.0 Hz, 1H), 4.50 (d, J=10.6 Hz, 1H), 4.43 (d, J=10.8 Hz, 1H), 4.31 (d, J=10.8 Hz, 1H), 4.23 (d, J=10.6 Hz, 1H), 4.03-3.96 (m, 2H), 3.94-3.87 (m, 2H), 3.78-3.70 (m, 2H), 3.69-3.61 (m, 2H), 3.51 (s, 3H), 2.66 (t, J=5.5 Hz, 1H), 2.64-2.57 (m, 1H), 2.30-2.23 (m, 4H), 1.91-1.60 (m, 8H), 1.51 (s, 9H), 0.89-0.85 (m, 18H), 0.60-0.51 (m, 3H).

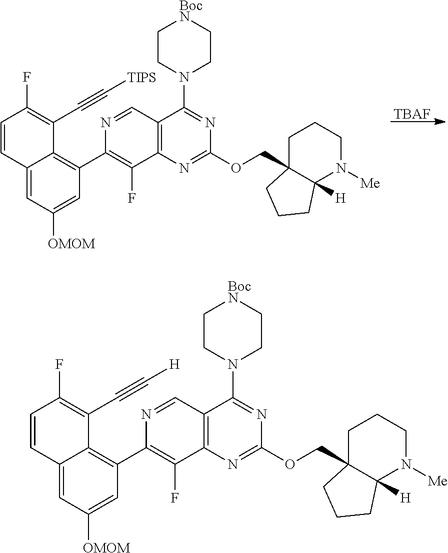

### Step 3: Preparation of tert-butyl 4-(7-(8-ethynyl-7-fluoro-3-(methoxymethoxy)naphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)piperazine-1-carboxylate

tert-Butyl 4-(8-fluoro-7-(7-fluoro-3-(methoxymethoxy)-8-((triisopropylsilyl)ethynyl)naphthalen-1-yl)-2-(((4aS,7aR)-1-methyloctahydro-4aH-cyclopenta[b]pyridin-4a-yl)methoxy)pyrido[4,3-d]pyrimidin-4-yl)piperazine-1-carboxylate (360 mg, 0.407 mmol) was dissolved in THE (4.1 mL), and the solution was cooled to 0° C. TBAF solution (1.0 M in THF, 0.6 mL, 0.6 mmol) was added dropwise and the reaction mixture was stirred for 5 min. The reaction mixture was directly concentrated, and the crude residue was purified by column chromatography (50→100% EtOAc with 5% Et₃N/hexanes) to provide the desired product (250 mg, 0.34 mmol, 84% yield over two steps). LC/MS (ESI) m/z: [M+H]⁺ calcd for C₄₀H₄₇F₂N₆O₅ 729.4; found 729.7; ¹H NMR (600 MHz, CDCl₃) δ 8.99 (s, 1H), 7.83 (dd, J=9.1, 5.6 Hz, 1H), 7.54 (d, J=2.5 Hz, 1H), 7.37 (t, J=2.2 Hz, 1H), 7.29 (t, J=8.7 Hz, 1H), 5.32 (d, J=6.9 Hz, 1H), 5.30 (d, J=6.9 Hz, 1H), 4.49 (dd, J=10.7, 6.7 Hz, 1H), 4.30 (dd, J=10.7, 2.5 Hz, 1H), 4.01-3.96 (m, 4H), 3.70 (br d, J=4.5 Hz, 4H), 3.52 (s, 3H), 2.81 (d, J=6.5 Hz, 1H), 2.70 (t, J=6.0 Hz, 1H), 2.31-2.28 (m, 1H), 2.27 (s, 3H), 1.90-1.64 (m, 11H), 1.51 (s, 9H).

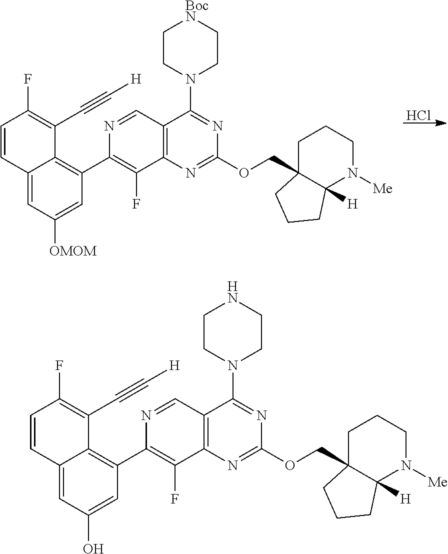

### Step 4: Preparation of 5-ethynyl-6-fluoro-4-(8-fluoro-2-(((4aS, 7aR)-1-methyloctahydro-4aH-cyclopenta[b]pyridin-4a-yl)methoxy)-4-(piperazin-1-yl)pyrido[4,3-d]pyrimidin-7-yl)naphthalen-2-ol

tert-Butyl 4-(7-(8-ethynyl-7-fluoro-3-(methoxymethoxy)naphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)piperazine-1-carboxylate (100 mg, 0.137 mmol) was dissolved in MeCN (1.4 mL) and the solution was cooled to 0° C. HCl solution (4.0 M in dioxane, 0.34 mL, 1.37 mmol) was added dropwise, and the reaction mixture was allowed to warm to room temperature. After 2 h, sodium hydroxide solution (1.0 M, 1.37 mL, 1.37 mmol) was added, and the solution was concentrated to remove volatile organics. The resulting aqueous solution was frozen and directly lyophilized to provide the desired product along with sodium chloride. The material was of sufficient purity to be used directly in the next step, without additional purification (quantitative yield assumed). LC/MS (ESI) m/z: [M+H]⁺ calcd for C₃₃H₃₅F₂N₆O₂ 585.3; found 585.5.

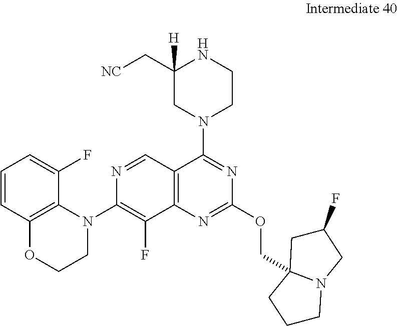

### 2-((S)-4-(8-fluoro-7-(5-fluoro-2,3-dihydro-4H-benzo[b][1,4]oxazin-4-yl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

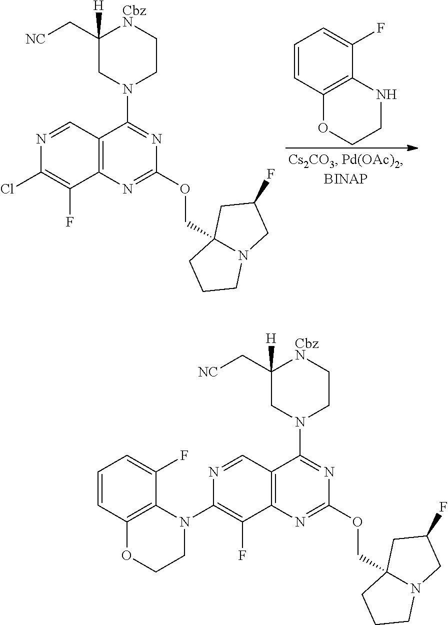 [SMILES: N#CC[C@H]1CN(c2nc(OC[C@@]34CCCN3C[C@H](F)C4)nc3c(F)c(Cl)ncc23)CCN1C(=O)OCc1ccccc1 ; Fc1cccc2c1NCCO2 ; N#CC[C@H]1CN(c2nc(OC[C@@]34CCCN3C[C@H](F)C4)nc3c(F)c(N4CCOc5cccc(F)c54)ncc23)CCN1C(=O)OCc1ccccc1]

### Step 1: Preparation of benzyl (S)-2-(cyanomethyl)-4-(8-fluoro-7-(5-fluoro-2,3-dihydro-4H-benzo[b][1,4]oxazin-4-yl)-2-(((2R, 7aS)-2-fluorotetrahydro-1H-pyrrolizin-7a(5H)-yl)methoxy)pyrido[4,3-d]pyrimidin-4-yl)piperazine-1-carboxylate

Benzyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (50 mg, 0.084 mmol), 5-fluoro-3,4-dihydro-2H-benzo[b][1,4]oxazine (26 mg, 0.17 mmol), cesium carbonate (82 mg, 0.25 mmol), BINAP (10.4 mg, 0.017 mmol), and palladium(II) acetate (1.9 mg, 8 μmol)were combined as solids in a microwave vial. The vial was sealed. The atmosphere was evacuated and replaced with nitrogen. This process was performed three times. Degassed dioxane (2.0 mL) was added and the reaction mixture was heated at 100° C. in the microwave for 16 h. The reaction mixture was filtered through a pad of diatomaceous earth (Celite©) and was concentrated. The crude residue was purified by column chromatography (20→100% EtOAc with 5% Et₃N/hexanes) to provide the desired product (15.8 mg, 0.022 mmol, 26% yield). LC/MS (ESI) m/z: [M+H]⁺ calcd for C₃₇H₃₈F₃N₈O₄ 715.3; found 715.2.

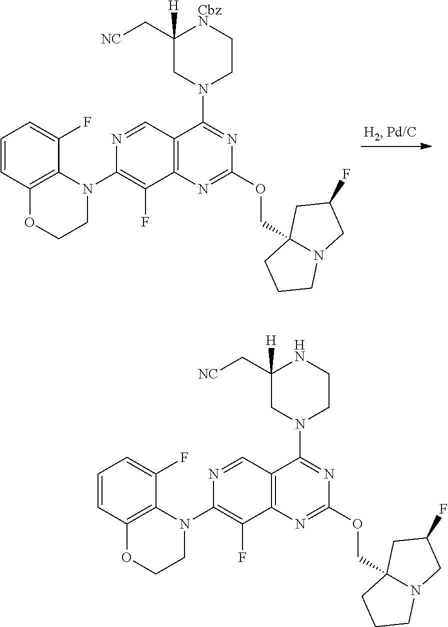 [SMILES: N#CC[C@H]1CN(c2nc(OC[C@@]34CCCN3C[C@H](F)C4)nc3c(F)c(N4CCOc5cccc(F)c54)ncc23)CCN1C(=O)OCc1ccccc1 ; N#CC[C@H]1CN(c2nc(OC[C@@]34CCCN3C[C@H](F)C4)nc3c(F)c(N4CCOc5cccc(F)c54)ncc23)CCN1]

### Step 2: Preparation of 2-((S)-4-(8-fluoro-7-(5-fluoro-2,3-dihydro-4H-benzo[b][1,4]oxazin-4-yl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

Benzyl (S)-2-(cyanomethyl)-4-(8-fluoro-7-(5-fluoro-2,3-dihydro-4H-benzo[b][1,4]oxazin-4-yl)-2-(((2R, 7aS)-2-fluorotetrahydro-1H-pyrrolizin-7a(5H)-yl)methoxy)pyrido[4,3-d]pyrimidin-4-yl)piperazine-1-carboxylate (16 mg, 0.022 mmol) was dissolved in MeOH (2.0 mL) and palladium on carbon (10 wt. %, 2 mg, 0.002 mmol) was added. Hydrogen gas was bubbled through the solution for 5 min then the reaction mixture was kept under an atmosphere of hydrogen (balloon) for 1.5 h. The reaction mixture was filtered through a plug of diatomaceous earth (Celite©) and was concentrated. The crude residue was used directly in the next step, without additional purification (quantitative yield assumed). LC/MS (ESI) m/z: [M+H]⁺ calcd for C₂₉H₃₂F₃N₈O₂ 581.3; found 581.5.

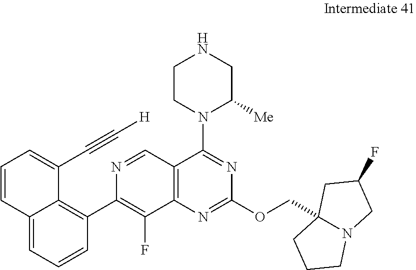

### 7-(8-ethynylnaphthalen-1-yl)-8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidine

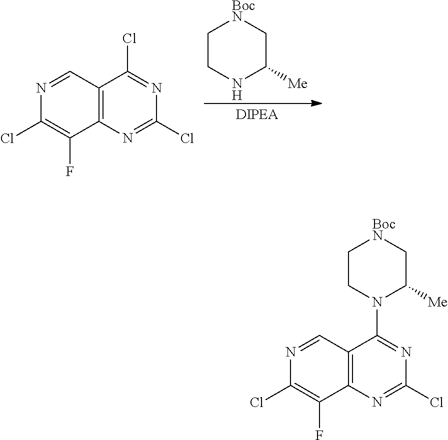 [SMILES: Fc1c(Cl)ncc2c(Cl)nc(Cl)nc12 ; C[C@H]1CN(C(=O)OC(C)(C)C)CCN1 ; C[C@H]1CN(C(=O)OC(C)(C)C)CCN1c1nc(Cl)nc2c(F)c(Cl)ncc12]

### Step 1: Preparation of tert-butyl (S)-4-(2,7-dichloro-8-fluoropyrido[4,3-d]pyrimidin-4-yl)-3-methylpiperazine-1-carboxylate

2,4,7-Trichloro-8-fluoropyrido[4,3-d]pyrimidine (2.5 g, 9.9 mmol) was dissolved in DCM (100 mL), and the solution was cooled to −40° C. DIPEA (2.6 mL, 15 mmol) was added dropise followed by tert-butyl (S)-3-methylpiperazine-1-carboxylate (2.0 g, 9.9 mmol). The reaction mixture was allowed to warm to room temperature and was stirred for 1 h. The solution was concentrated, and the crude residue was purified by column chromatography (0→60% EtOAc/hexanes) to provide the desired product (3.7 g, 8.8 mmol, 89% yield). LC/MS (ESI) m/z: [M+H]⁺ calcd for C₁₇H₂₁C₁₂FN₅O₂ 416.1; found 416.0; ¹H NMR (500 MHz, CDCl₃) δ 8.85 (s, 1H), 4.94-4.86 (m, 1H), 4.52-4.42 (m, 1H), 4.31-4.09 (m, 1H), 4.07-3.95 (m, 1H), 3.78-3.66 (m, 1H), 3.31-3.07 (m, 2H), 1.54 (d, J=6.8 Hz, 3H), 1.52 (s, 9H).

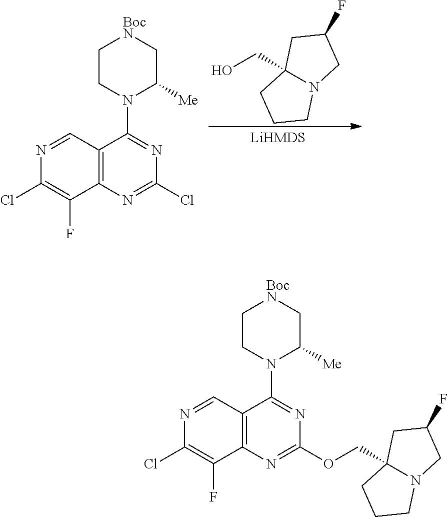 [SMILES: C[C@H]1CN(C(=O)OC(C)(C)C)CCN1c1nc(Cl)nc2c(F)c(Cl)ncc12 ; OC[C@@]12CCCN1C[C@H](F)C2 ; C[C@H]1CN(C(=O)OC(C)(C)C)CCN1c1nc(OC[C@@]23CCCN2C[C@H](F)C3)nc2c(F)c(Cl)ncc12]

### Step 2: Preparation of tert-butyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazine-1-carboxylate

((2R, 7aS)-2-Fluorotetrahydro-1H-pyrrolizin-7a(5H)-yl)methanol (1.61 g, 10.1 mmol) was dissolved in THE (45 mL), and the solution was cooled to 0° C. LiHMDS solution (1.0 M in THF, 10.6 mL, 10.6 mmol) was added dropwise, and the resulting mixture was stirred for 10 min. tert-Butyl (S)-4-(2,7-dichloro-8-fluoropyrido[4,3-d]pyrimidin-4-yl)-3-methylpiperazine-1-carboxylate (3.7 g, 8.8 mmol) as a solution in THF (50 mL) was added dropwise. The reaction mixture was allowed to warm to room temperature and was stirred for 16 h. The solution was partially concentrated, and the crude residue was purified by column chromatography (50→100% EtOAc with 5% Et₃N/hexanes) to provide the desired product (4.55 g, 8.4 mmol, 96% yield) as a yellow foam. LC/MS (ESI) m/z: [M+H]⁺ calcd for C₂₅H₃₄C₁F₂N₆O₃ 539.2; found 539.5; ¹H NMR (600 MHz, CDCl₃) δ 8.70 (s, 1H), 0.00 (d, J=52.7 Hz, 1H), 4.78 (br s, 1H), 4.33 (br d, J=13.1 Hz, 1H), 4.25 (d, J=10.4 Hz, 1H), 4.15 (d, J=10.4 Hz, 1H), 4.26-3.82 (m, 2H), 3.67-3.56 (m, 1H), 3.28-2.94 (m, 6H), 2.29-2.09 (m, 3H), 1.99-1.82 (m, 3H), 1.49 (s, 9H), 1.46 (d, J=6.7 Hz, 3H).

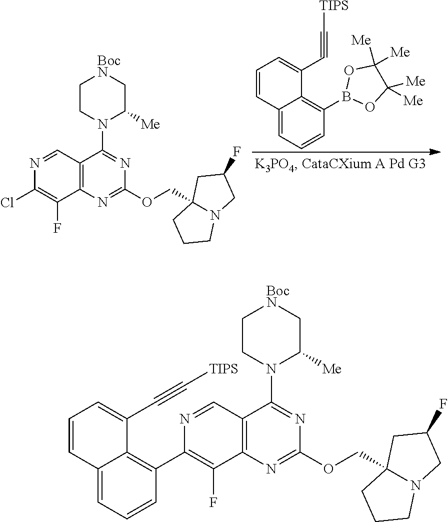

### Step 3: Preparation of tert-butyl (S)-4-(8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)-3-methylpiperazine-1-carboxylate

tert-Butyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazine-1-carboxylate (45 mg, 0.083 mmol), triisopropyl((8-(4,4,5,5-tetramethyl-1,3,2-dioxaborolan-2-yl)naphthalen-1-yl)ethynyl)silane (54 mg, 0.13 mmol), and methanesulfonato(diadamantyl-n-butylphosphino)-2′-amino-1,1′-biphenyl-2-yl)palladium(II) dichloromethane adduct, [cataCXium A Palladacycle Gen. 3] (3.0 mg, 4.2 μmol) were combined as solids in a microwave vial. The vial was sealed. The atmosphere was evacuated and replaced with nitrogen. This process was performed three times. Degassed dioxane (400 μL) and potassium phosphate solution (2.0 M in water, 125 μL, 0.25 mmol) were added, and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (66 mg, 0.081 mmol, 97% yield). LC/MS (ESI) m/z: [M+H]⁺ calcd for C₄₆H₆₁F₂N₆O₃Si 811.4; found 811.4; ¹H NMR (600 MHz, CDCl₃) δ 9.07-8.96 (m, 1H), 7.99-7.89 (m, 2H), 7.84-7.77 (m, 1H), 7.60-7.51 (m, 2H), 7.48-7.43 (m, 1H), 5.35-5.20 (m, 1H), 5.05-4.67 (m, 1H), 4.55-3.70 (m, 6H), 3.58-2.95 (m, 6H), 2.34-2.09 (m, 3H), 1.97-1.84 (m, 3H), 1.55-1.49 (m, 12H), 0.91-0.83 (m, 18H), 0.60-0.48 (m, 3H).

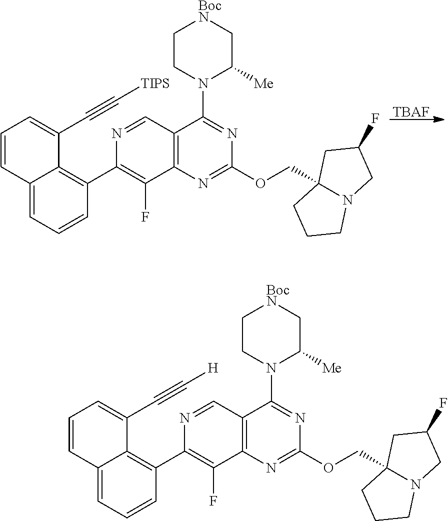

### Step 4: Preparation of tert-butyl (S)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazine-1-carboxylate

tert-Butyl (S)-4-(8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)-3-methylpiperazine-1-carboxylate (66 mg, 0.081 mmol)was dissolved in THE (800 μL) and the solution was cooled to 0° C. TBAF solution (1.0 M in THF, 120 μL, 0.12 mmol) was added dropwise, and the reaction mixture was warmed to room temperature. After 5 min, the reaction mixture was concentrated, and the crude residue was purified by column chromatography (50→100% EtOAc with 5% Et₃N/hexanes) to provide the desired product (53 mg, 0.08 mmol, 99% yield) LC/MS (ESI) m/z: [M+H]⁺ calcd for C₃₇H₄₁F₂N₆O₃ 655.3; found 655.4; ¹H NMR (600 MHz, CDCl₃) δ 9.01-8.93 (m, 1H), 8.01-7.93 (m, 2H), 7.77-7.73 (m, 1H), 7.63-7.55 (m, 2H), 7.49-7.43 (m, 1H), 5.28 (d, J=53.6 Hz, 1H), 4.93-4.78 (m, 1H), 4.49-4.36 (m, 1H), 4.31-4.25 (m, 1H), 4.21-4.16 (m, 1H), 4.30-3.87 (m, 2H), 3.70-3.59 (m, 1H), 3.35-2.94 (m, 6H), 2.30-2.13 (m, 3H), 1.97-1.87 (m, 4H), 1.51-1.46 (m, 12H).

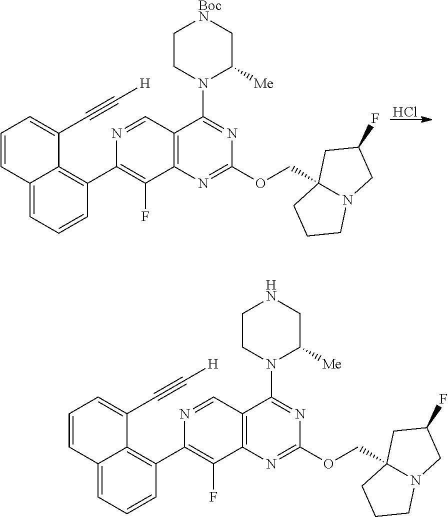 [SMILES: C#Cc1cccc2cccc(-c3ncc4c(N5CCN(C(=O)OC(C)(C)C)C[C@@H]5C)nc(OC[C@@]56CCCN5C[C@H](F)C6)nc4c3F)c12 ; Cl ; C#Cc1cccc2cccc(-c3ncc4c(N5CCNC[C@@H]5C)nc(OC[C@@]56CCCN5C[C@H](F)C6)nc4c3F)c12]

### Step 5: Preparation of 7-(8-ethynylnaphthalen-1-yl)-8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidine

tert-Butyl (S)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazine-1-carboxylate (59 mg, 0.090 mmol) was dissolved in MeCN (900 μL), and the solution was cooled to 0° C. HCl solution (4.0 M in dioxane, 230 μL, 0.90 mmol) was added dropwise. After 2 h, 1-methylimidazole (100 μL) was added, and the reaction mixture was concentrated. The crude residue was used directly in the next step, without additional purification (quantitative yield assumed). LC/MS (ESI) m/z: [M+H]⁺ calcd for C₃₂H₃₃F₂N₆O 555.3; found 555.2.

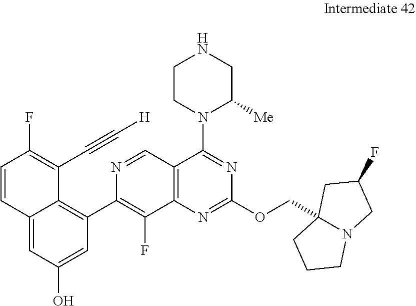

### 5-ethynyl-6-fluoro-4-(8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidin-7-yl)naphthalen-2-ol

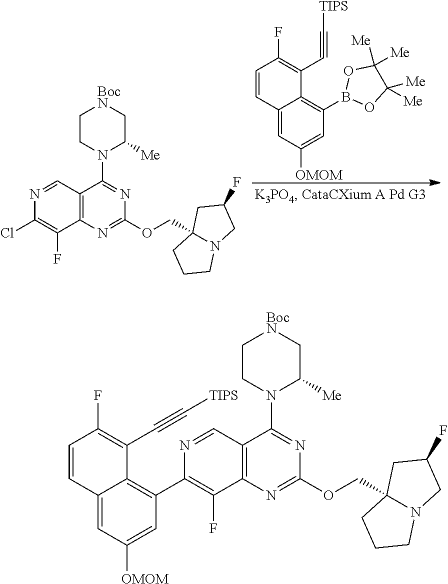

### Step 1: Preparation of tert-butyl (S)-4-(8-fluoro-7-(7-fluoro-3-(methoxymethoxy)-8-((triisopropylsilyl)ethynyl)naphthalen-1-yl)-2-(((2R, 7aS)-2-fluorotetrahydro-1H-pyrrolizin-7a(5H)-yl)methoxy)pyrido[4,3-d]pyrimidin-4-yl)-3-methylpiperazine-1-carboxylate

tert-Butyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazine-1-carboxylate (100 mg, 0.19 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (140 mg, 0.28 mmol), and methanesulfonato(diadamantyl-n-butylphosphino)-2′-amino-1,1′-biphenyl-2-yl)palladium(II) dichloromethane adduct, [cataCXium A Palladacycle Gen. 3] (6.8 mg, 9.3 μmol) were combined as solids in a microwave vial. The vial was sealed. The atmosphere was evacuated and replaced with nitrogen. This process was performed three times. Degassed dioxane (900 μL) and potassium phosphate solution (2.0 M in water, 270 μL, 0.54 mmol) were added, and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (160 mg, 0.18 mmol, 97% yield). LCMS (ESI) m/z: [M+H]* calcd for C₄₈H₆₄F₃N₆O₅Si 889.5; found 889.5.

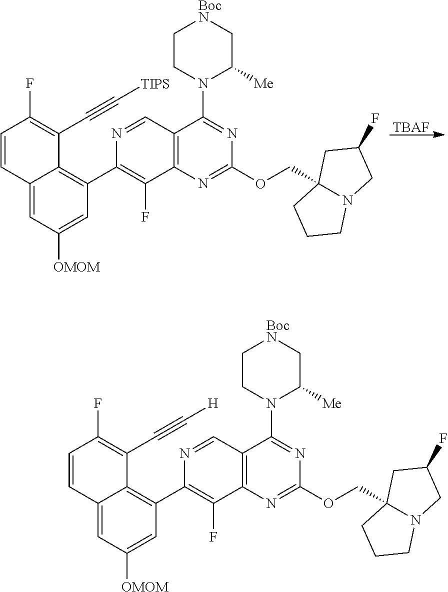

### Step 2: Preparation of tert-butyl (S)-4-(7-(8-ethynyl-7-fluoro-3-(methoxymethoxy)naphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazine-1-carboxylate

tert-Butyl (S)-4-(8-fluoro-7-(7-fluoro-3-(methoxymethoxy)-8-((triisopropylsilyl)ethynyl)naphthalen-1-yl)-2-(((2R, 7aS)-2-fluorotetrahydro-1H-pyrrolizin-7a(5H)-yl)methoxy)pyrido[4,3-d]pyrimidin-4-yl)-3-methylpiperazine-1-carboxylate (160 mg, 0.180 mmol) was dissolved in THE (1.8 mL), and the solution was cooled to 0° C. TBAF solution (1.0 M in THF, 180 μL, 0.18 mmol) was added dropwise, and the reaction mixture was warmed to room temperature. After 5 min, the reaction mixture was concentrated, and the crude residue was purified by column chromatography (50→100% EtOAc with 5% Et₃N/hexanes) to provide the desired product (128 mg, 0.175 mmol, 97% yield). LCMS (ESI) m/z: [M+H]⁺ calcd for C₃₉H₄₄F₃N₆O₅ 733.3; found 733.3; ¹H NMR (500 MHz, CDCl₃) δ 8.99-8.91 (m, 1H), 7.86-7.80 (m, 1H), 7.55-7.52 (m, 1H), 7.39-7.35 (m, 1H), 7.31-7.26 (m, 1H), 5.32 (d, J=6.8 Hz, 1H), 5.29 (d, J=6.8 Hz, 1H), 5.28 (d, J=54.6 Hz, 1H), 4.93-4.80 (m, 1H), 4.45-4.35 (m, 1H), 4.32-4.23 (m, 1H), 4.21-4.15 (m, 1H), 4.00-3.89 (m, 1H), 3.71-3.58 (m, 1H), 3.52 (s, 3H), 3.34-3.04 (m, 5H), 3.02-2.93 (m, 1H), 2.31-2.11 (m, 3H), 1.99-1.84 (m, 5H), 1.52-1.45 (m, 12H).

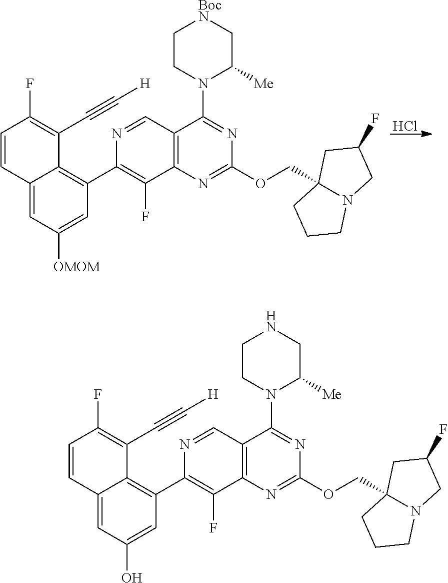

### Step 3: Preparation of 5-ethynyl-6-fluoro-4-(8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidin-7-yl)naphthalen-2-ol

tert-Butyl (S)-4-(7-(8-ethynyl-7-fluoro-3-(methoxymethoxy)naphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazine-1-carboxylate (120 mg, 0.164 mmol) was dissolved in MeCN (1.6 mL), and the solution was cooled to 0° C. HCl solution (4.0 M in dioxane, 410 μL, 1.6 mmol) was added dropwise. After 2 h, 1-methylimidazole (200 μL) was added, and the reaction mixture was concentrated. The crude residue was used directly in the next step, without additional purification (quantitative yield assumed). LCMS (ESI) m/z: [M+H]⁺ calcd for C₃₂H₃₂F₃N₆O₂ 589.2; found 589.2.

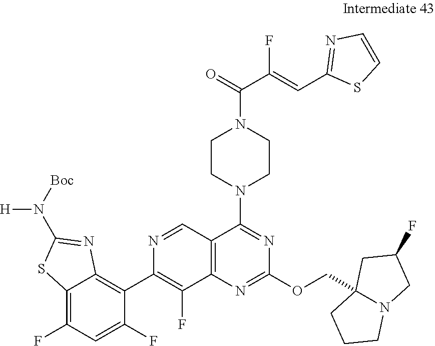

### tert-butyl (5,7-difluoro-4-(8-fluoro-4-(4-((Z)-2-fluoro-3-(thiazol-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7-yl)benzo[d]thiazol-2-yl)carbamate

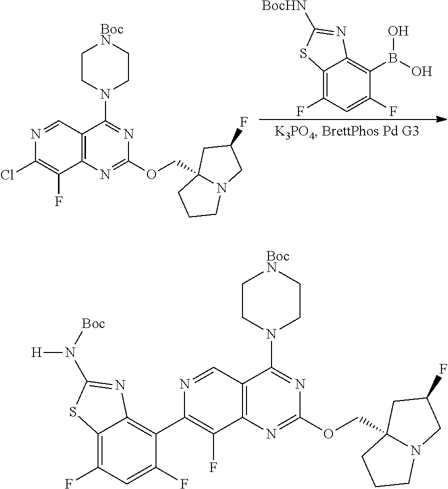 [SMILES: CC(C)(C)OC(=O)N1CCN(c2nc(OC[C@@]34CCCN3C[C@H](F)C4)nc3c(F)c(Cl)ncc23)CC1 ; CC(C)(C)OC(=O)Nc1nc2c(B(O)O)c(F)cc(F)c2s1 ; CC(C)(C)OC(=O)Nc1nc2c(-c3ncc4c(N5CCN(C(=O)OC(C)(C)C)CC5)nc(OC[C@@]56CCCN5C[C@H](F)C6)nc4c3F)c(F)cc(F)c2s1]

### Step 1: Preparation of tert-butyl 4-(7-(2-((tert-butoxycarbonyl)amino)-5,7-difluorobenzo[d]thiazol-4-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate

tert-Butyl 4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (20 mg, 0.038 mmol), (2-((tert-butoxycarbonyl)amino)-5,7-difluorobenzo[d]thiazol-4-yl)boronic acid (19 mg, 0.057 mmol) [commercial], and [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate [BrettPhos Pd G3] (3.5 mg, 0.0038 mmol) were combined as solids in a microwave vial. The vial was sealed. The atmosphere was evacuated and replaced with nitrogen. This process was performed three times. Degassed dioxane (400 μL) and potassium phosphate solution (2.0 M in water, 60 μL, 0.11 mmol) were added, and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (16 mg, 0.020 mmol, 54% yield). LCMS (ESI) m/z: [M+H]⁺ calcd for C₃₆H₄₃F₄N₈O₅S 775.3; found 775.3; ¹H NMR (600 MHz, CDCl₃) δ 9.08 (s, 1H), 8.17 (br s, 1H), 6.95 (t, J=9.4 Hz, 1H), 5.27 (d, J=54.3 Hz, 1H), 4.29 (br d, J=10.5 Hz, 1H), 4.19 (d, J=10.4 Hz, 1H), 4.00-3.93 (m, 4H), 3.69-3.63 (m, 4H), 3.28-3.24 (m, 1H), 3.23-3.19 (m, 1H), 3.17 (s, 1H), 3.03-2.92 (m, 1H), 2.30-2.11 (m, 3H), 1.99-1.85 (m, 3H), 1.54 (s, 9H), 1.50 (s, 9H).

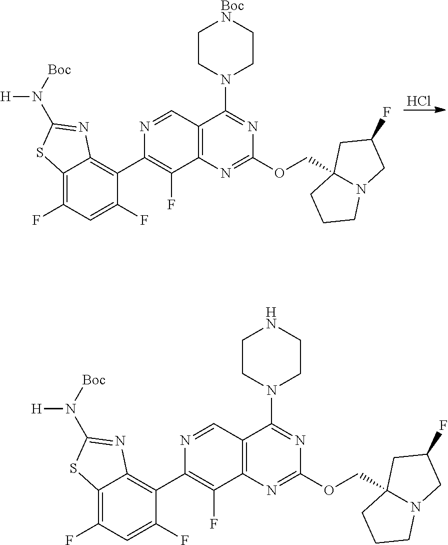 [SMILES: CC(C)(C)OC(=O)Nc1nc2c(-c3ncc4c(N5CCN(C(=O)OC(C)(C)C)CC5)nc(OC[C@@]56CCCN5C[C@H](F)C6)nc4c3F)c(F)cc(F)c2s1 ; Cl ; CC(C)(C)OC(=O)Nc1nc2c(-c3ncc4c(N5CCNCC5)nc(OC[C@@]56CCCN5C[C@H](F)C6)nc4c3F)c(F)cc(F)c2s1]

### Step 2: Preparation of tert-butyl (5,7-difluoro-4-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)benzo[d]thiazol-2-yl)carbamate

tert-Butyl 4-(7-(2-((tert-butoxycarbonyl)amino)-5,7-difluorobenzo[d]thiazol-4-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (16 mg, 0.021 mmol) was dissolved in MeCN (1.0 mL), and HCl solution (4.0 M in dioxane, 50 μL, 0.2 mmol) was added dropwise. The reaction mixture was stirred at room temperature for 20 h. 1-methylimidazole (50 μL) was added, and the solution was concentrated. The crude residue was used directly in the next step, without additional purification (quantitative yield assumed). LCMS (ESI) m/z: [M+H]⁺ calcd for C₃₁H₃₅F₄N₈O₃S 675.2; found 675.2.

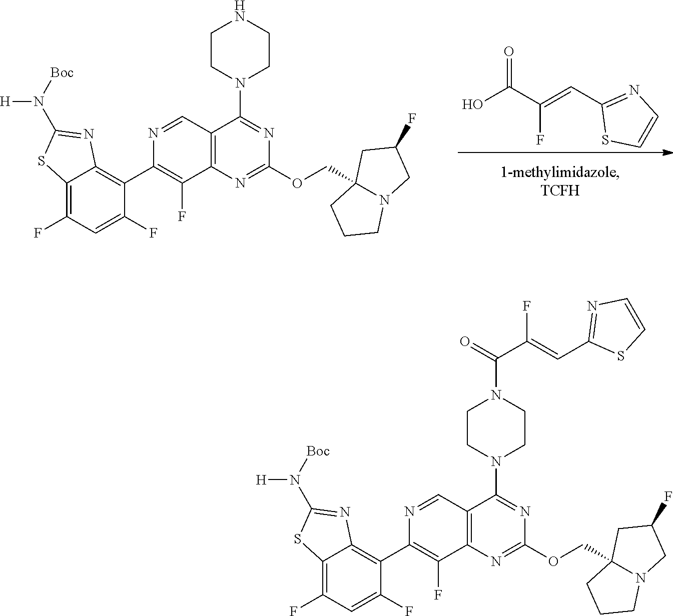 [SMILES: CC(C)(C)OC(=O)Nc1nc2c(-c3ncc4c(N5CCNCC5)nc(OC[C@@]56CCCN5C[C@H](F)C6)nc4c3F)c(F)cc(F)c2s1 ; O=C(O)/C(F)=C/c1nccs1 ; CC(C)(C)OC(=O)Nc1nc2c(-c3ncc4c(N5CCN(C(=O)/C(F)=C/c6nccs6)CC5)nc(OC[C@@]56CCCN5C[C@H](F)C6)nc4c3F)c(F)cc(F)c2s1]

### Step 3: Preparation of tert-butyl (5,7-difluoro-4-(8-fluoro-4-(4-((Z)-2-fluoro-3-(thiazol-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7-yl)benzo[d]thiazol-2-yl)carbamate

tert-Butyl (5,7-difluoro-4-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)benzo[d]thiazol-2-yl)carbamate (13 mg, 0.019 mmol) and (Z)-2-fluoro-3-(thiazol-2-yl)acrylic acid (10 mg, 0.06 mmol) were combined as solids and dissolved in MeCN (400 L). 1-methylimidazole (9 μL, 0.19 mmol) was added followed by chloro-N,N,N′,N′-tetramethylformamidinium hexafluorophosphate (36 mg, 0.127 mmol). The reaction mixture was stirred for 5 min and was concentrated. The crude residue was used directly in the next step, without additional purification (quantitative yield assumed). LCMS (ESI) m/z: [M+H]⁺ calcd for C₃₇H₃₇F₅N₉O₄S₂ 830.2; found 830.3.

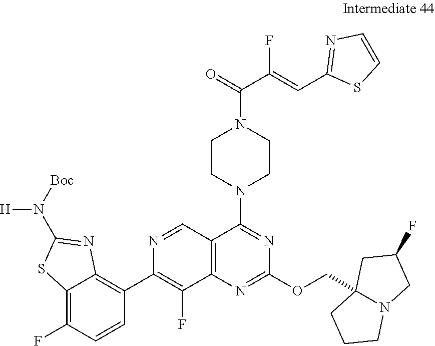

### tert-butyl (5,7-difluoro-4-(8-fluoro-4-(4-((Z)-2-fluoro-3-(thiazol-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7-yl)benzo[d]thiazol-2-yl)carbamate

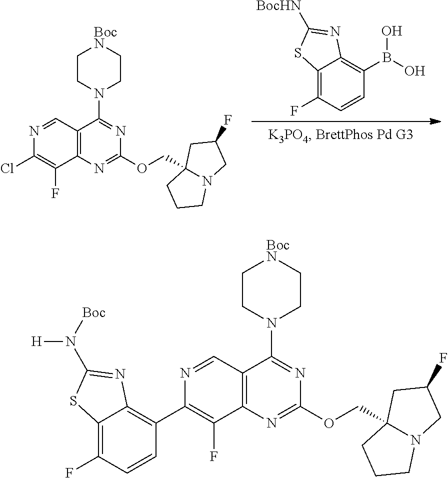 [SMILES: CC(C)(C)OC(=O)N1CCN(c2nc(OC[C@@]34CCCN3C[C@H](F)C4)nc3c(F)c(Cl)ncc23)CC1 ; CC(C)(C)OC(=O)Nc1nc2c(B(O)O)ccc(F)c2s1 ; CC(C)(C)OC(=O)Nc1nc2c(-c3ncc4c(N5CCN(C(=O)OC(C)(C)C)CC5)nc(OC[C@@]56CCCN5C[C@H](F)C6)nc4c3F)ccc(F)c2s1]

### Step 1: Preparation of tert-butyl 4-(7-(2-((tert-butoxycarbonyl)amino)-7-fluorobenzo[d]thiazol-4-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate

tert-Butyl 4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (30 mg, 0.057 mmol), (2-((tert-butoxycarbonyl)amino)-7-fluorobenzo[d]thiazol-4-yl)boronic acid (27 mg, 0.086 mmol) [commercial], and [(2-dicyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate [BrettPhos Pd G3] (5.2 mg, 0.0057 mmol) were combined as solids in a microwave vial. The vial was sealed. The atmosphere was evacuated and replaced with nitrogen. This process was performed three times. Degassed dioxane (600 μL) and potassium phosphate solution (2.0 M in water, 86 μL, 0.17 mmol) were added, and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (25 mg, 0.033 mmol, 58% yield). LCMS (ESI) m/z: [M+H]⁺ calcd for C₃₆H₄₄F₃N₈O₅S 757.3; found 757.6; ¹H NMR (600 MHz, CDCl₃) δ 9.08 (s, 1H), 8.17 (br s, 1H), 6.95 (t, J=9.4 Hz, 1H), 5.27 (d, J=54.3 Hz, 1H), 4.29 (br d, J=10.5 Hz, 1H), 4.19 (d, J=10.4 Hz, 1H), 4.00-3.93 (m, 4H), 3.69-3.63 (m, 4H), 3.28-3.24 (m, 1H), 3.23-3.19 (m, 1H), 3.17 (s, 1H), 3.03-2.92 (m, 1H), 2.30-2.11 (m, 3H), 1.99-1.85 (m, 3H), 1.54 (s, 9H), 1.50 (s, 9H).

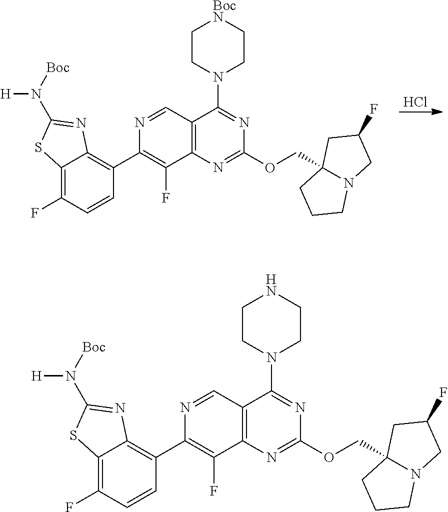 [SMILES: CC(C)(C)OC(=O)Nc1nc2c(-c3ncc4c(N5CCN(C(=O)OC(C)(C)C)CC5)nc(OC[C@@]56CCCN5C[C@H](F)C6)nc4c3F)ccc(F)c2s1 ; Cl ; CC(C)(C)OC(=O)Nc1nc2c(-c3ncc4c(N5CCNCC5)nc(OC[C@@]56CCCN5C[C@H](F)C6)nc4c3F)ccc(F)c2s1]

### Step 2: Preparation of tert-butyl (7-fluoro-4-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)benzo[d]thiazol-2-yl)carbamate

tert-Butyl 4-(7-(2-((tert-butoxycarbonyl)amino)-7-fluorobenzo[d]thiazol-4-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (25 mg, 0.033 mmol) was dissolved in MeCN (1.6 mL), and HCl solution (4.0 M in dioxane, 83 μL, 0.33 mmol) was added dropwise. The reaction mixture was stirred at room temperature for 2 h. 1-methylimidazole (50 μL) was added, and the solution was concentrated. The crude residue was used directly in the next step, without additional purification (quantitative yield assumed). LCMS (ESI) m/z: [M+H]⁺ calcd for C₃₁H₃₆F₃N₈O₃S 657.3; found 657.2.

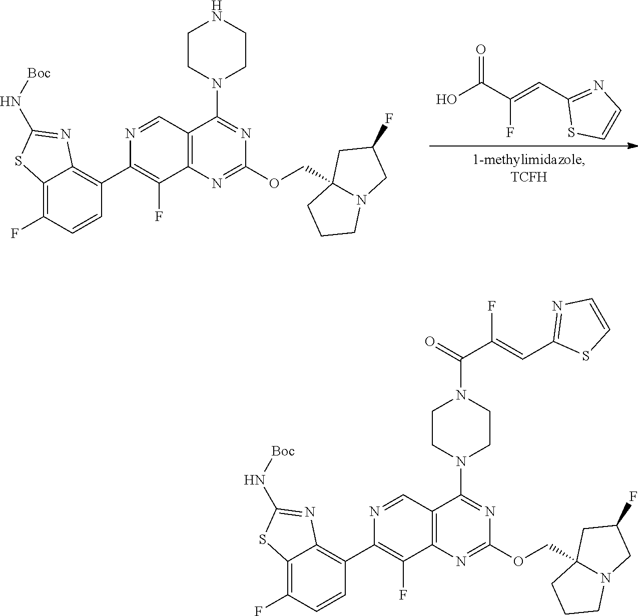 [SMILES: CC(C)(C)OC(=O)Nc1nc2c(-c3ncc4c(N5CCNCC5)nc(OC[C@@]56CCCN5C[C@H](F)C6)nc4c3F)ccc(F)c2s1 ; O=C(O)/C(F)=C/c1nccs1 ; CC(C)(C)OC(=O)Nc1nc2c(-c3ncc4c(N5CCN(C(=O)/C(F)=C/c6nccs6)CC5)nc(OC[C@@]56CCCN5C[C@H](F)C6)nc4c3F)ccc(F)c2s1]

### Step 3: Preparation of tert-butyl (7-fluoro-4-(8-fluoro-4-(4-((Z)-2-fluoro-3-(thiazol-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7-yl)benzo[d]thiazol-2-yl)carbamate

tert-Butyl (7-fluoro-4-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)benzo[d]thiazol-2-yl)carbamate (21 mg, 0.032 mmol)and (Z)-2-fluoro-3-(thiazol-2-yl)acrylic acid (17 mg, 0.096 mmol) were combined as solids and dissolved in MeCN (650 μL). 1-methylimidazole (15 μL, 0.32 mmol) was added followed by chloro-N,N,N′,N′-tetramethylformamidinium hexafluorophosphate (27 mg, 0.096 mmol). The reaction mixture was stirred for 5 min and was concentrated. The crude residue was used directly in the next step, without additional purification (quantitative yield assumed). LCMS (ESI) m/z: [M+H] calcd for C₃₇H₃₈F₄N₉O₄S₂ 812.2k found 812.2.

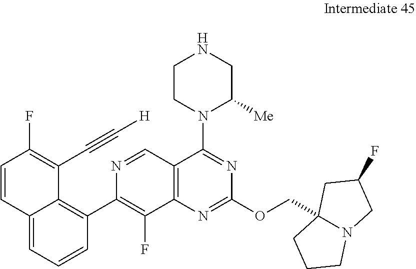

### 7-(8-ethynyl-7-fluoronaphthalen-1-yl)-8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidine

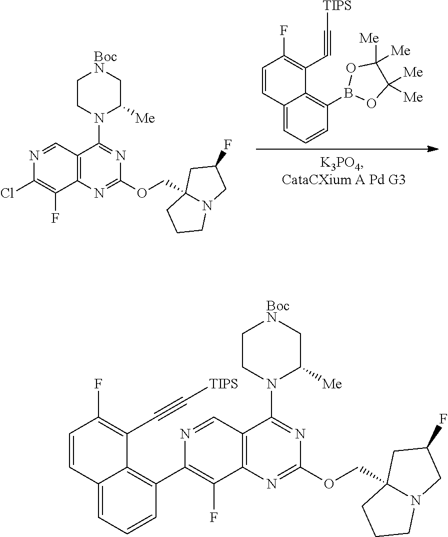

### Step 1: Preparation of tert-butyl (S)-4-(8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)-3-methylpiperazine-1-carboxylate

tert-Butyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazine-1-carboxylate (25 mg, 0.046 mmol), ((2-fluoro-8-(4,4,5,5-tetramethyl-1,3,2-dioxaborolan-2-yl)naphthalen-1-yl)ethynyl)triisopropylsilane (31.5 mg, 0.070 mmol), and methanesulfonato(diadamantyl-n-butylphosphino)-2′-amino-1,1′-biphenyl-2-yl)palladium(II) dichloromethane adduct, [cataCXium A Palladacycle Gen. 3] (1.7 mg, 2.1 μmol) were combined as solids in a microwave vial. The vial was sealed. The atmosphere was evacuated and replaced with nitrogen. This process was performed three times. Degassed dioxane (400 μL) and potassium phosphate solution (2.0 M in water, 70 μL, 0.14 mmol) were added, and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (30 mg, 0.036 mmol, 78% yield). LCMS (ESI) m/z: [M+H]⁺ calcd for C₄₆H₆₀F₃N₆O₃Si 829.4; found 829.5; ¹H NMR (600 MHz, CDCl₃) δ 9.05-8.97 (m, 1H), 7.96-7.93 (m, 1H), 7.92-7.88 (m, 1H), 7.59-7.51 (m, 2H), 7.36-7.31 (m, 1H), 5.39-5.19 (m, 1H), 5.06-4.65 (m, 1H), 4.43 (s, 1H), 4.32-3.69 (m, 4H), 3.65-3.48 (m, 1H), 3.44-2.91 (m, 6H), 2.34-2.10 (m, 3H), 1.97-1.85 (m, 3H), 1.52-1.49 (m, 12H), 0.89-0.85 (m, 18H), 0.61-0.50 (m, 3H).

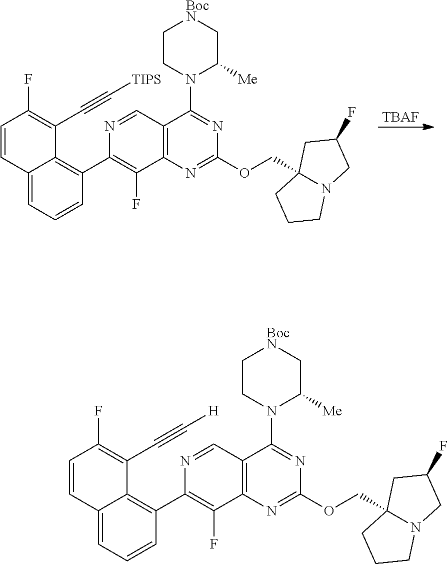

### Step 2: Preparation of tert-butyl (S)-4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazine-1-carboxylate

tert-Butyl (S)-4-(8-fluoro-7-(7-fluoro-8-((triisopropylsilyl)ethynyl)naphthalen-1-yl)-2-(((2R, 7aS)-2-fluorotetrahydro-1H-pyrrolizin-7a(5H)-yl)methoxy)pyrido[4,3-d]pyrimidin-4-yl)-3-methylpiperazine-1-carboxylate (30 mg, 0.036 mmol) was dissolved in THE (400 μL) and the solution was cooled to 0° C. TBAF solution (1.0 M in THF, 54 μL, 0.54 mmol) was added dropwise, and the reaction mixture was warmed to room temperature. After 5 min, the reaction mixture was concentrated, and the crude residue was purified by column chromatography (50→100% EtOAc with 5% Et₃N/hexanes) to provide the desired product (24 mg, 0.0 mmol, 99% yield). LCMS (ESI) m/z: [M+H]⁺ calcd for C₃₇H₄₀F₃N₆O₃ 673.3; found 673.6; ¹H NMR (600 MHz, CDCl₃) δ 9.00-8.94 (m, 1H), 7.99-7.92 (m, 2H), 7.66-7.55 (m, 2H), 7.37-7.30 (m, 1H), 5.28 (d, J=53.6 Hz, 1H), 4.93-4.76 (m, 1H), 4.52-3.86 (m, 5H), 3.73-3.55 (m, 1H), 3.34-2.83 (m, 7H), 2.35-2.10 (m, 3H), 2.00-1.81 (m, 3H), 1.52-1.49 (m, 12H).

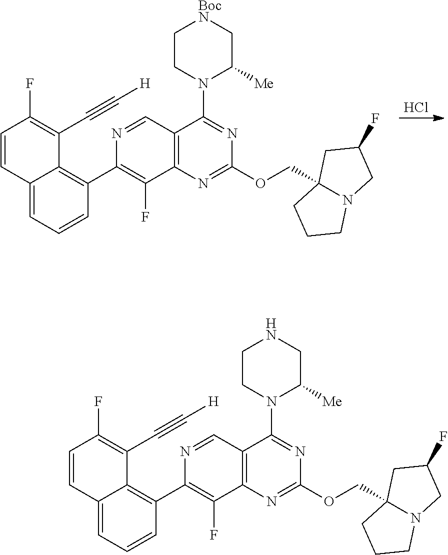 [SMILES: C#Cc1c(F)ccc2cccc(-c3ncc4c(N5CCN(C(=O)OC(C)(C)C)C[C@@H]5C)nc(OC[C@@]56CCCN5C[C@H](F)C6)nc4c3F)c12 ; Cl ; C#Cc1c(F)ccc2cccc(-c3ncc4c(N5CCNC[C@@H]5C)nc(OC[C@@]56CCCN5C[C@H](F)C6)nc4c3F)c12]

### Step 3: Preparation of 7-(8-ethynylnaphthalen-1-yl)-8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidine

tert-Butyl (S)-4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazine-1-carboxylate (24 mg, 0.036 mmol) was dissolved in MeCN (350 μL), and the solution was cooled to 0° C. HCl solution (4.0 M in dioxane, 89 μL, 0.36 mmol) was added dropwise. After 2 h, 1-methylimidazole (50 μL) was added, and the reaction mixture was concentrated. The crude residue was used directly in the next step, without additional purification (quantitative yield assumed). LCMS (ESI) m/z: [M+H]⁺ calcd for C₃₂H₃₂F₃N₆O 573.3; found 573.2.

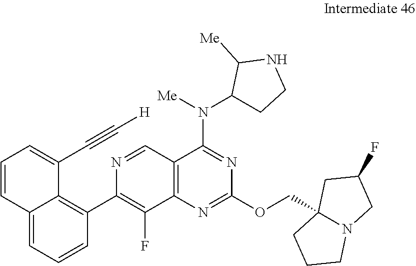 [SMILES: C#Cc1cccc2cccc(-c3ncc4c(N(C)C5CCNC5C)nc(OC[C@@]56CCCN5C[C@H](F)C6)nc4c3F)c12]

### 7-(8-ethynylnaphthalen-1-yl)-8-fluoro-2-(((2R, 7aS)-2-fluorotetrahydro-1H-pyrrolizin-7a(5H)-yl)methoxy)-N-methyl-N-(2-methylpyrrolidin-3-yl)pyrido[4,3-d]pyrimidin-4-amine

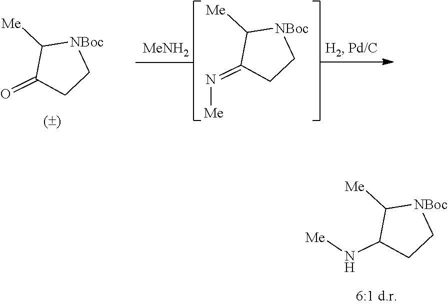

### Step 1: Preparation of tert-butyl 2-methyl-3-(methylamino)pyrrolidine-1-carboxylate

tert-Butyl 2-methyl-3-oxopyrrolidine-1-carboxylate (1 g, 5.02 mmol) was dissolved in methylamine solution (33 wt % in EtOH, 19 ml, 151 mmol), and the reaction mixture was stirred at room temperature for 20 h. The solution was concentrated, and the crude residue was resuspended in MeOH (50 mL). Palladium on carbon (10 wt. %, 0.5 g, 0.47 mmol) was added, and hydrogen gas (1 atm, balloon) was sparged through the solution for 5 min. The reaction mixture was stirred under an atmosphere of hydrogen for 20 h. The black suspension was filtered through a pad of diatomaceous earth (Celite®), eluting with additional MeOH, and the filtrate was concentrate. The crude residue was used directly in the next step, without further purification (quantitative yield assumed).

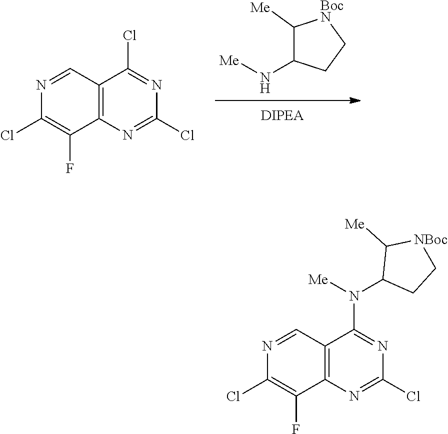 [SMILES: Fc1c(Cl)ncc2c(Cl)nc(Cl)nc12 ; CNC1CCN(C(=O)OC(C)(C)C)C1C ; CC1C(N(C)c2nc(Cl)nc3c(F)c(Cl)ncc23)CCN1C(=O)OC(C)(C)C]

### Step 2: Preparation of 2,7-dichloro-8-fluoro-N-methyl-N-(2-methylpyrrolidin-3-yl)pyrido[4,3-d]pyrimidin-4-amine

2,4,7-Trichloro-8-fluoropyrido[4,3-d]pyrimidine (1.2 g, 4.75 mmol) was suspended in DCM (24 mL) and the mixture was cooled to −5° C. (wet ice/acetone). DIPEA (0.83 mL, 4.75 mmol) was added followed by tert-butyl 2-methyl-3-(methylamino)pyrrolidine-1-carboxylate (1.0 g, 4.75 mmol). The reaction mixture was stirred for 5 min and was concentrated. The crude residue was used directly in the next step, without additional purification (quantitative yield assumed).

LC/MS (ESI) m/z: [M+H]⁺ calcd for C₁₈H₂₃C₁₂FN₅O₂ 430.1; found 430.1.

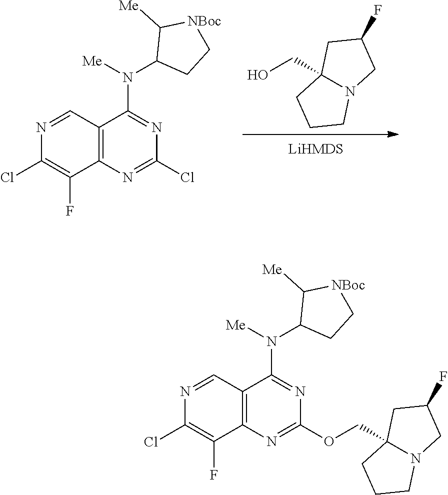 [SMILES: CC1C(N(C)c2nc(Cl)nc3c(F)c(Cl)ncc23)CCN1C(=O)OC(C)(C)C ; OC[C@@]12CCCN1C[C@H](F)C2 ; CC1C(N(C)c2nc(OC[C@@]34CCCN3C[C@H](F)C4)nc3c(F)c(Cl)ncc23)CCN1C(=O)OC(C)(C)C]

### Step 3: Preparation of tert-butyl 3-((7-chloro-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidine-1-carboxylate

tert-Butyl 3-((2,7-dichloro-8-fluoropyrido[4,3-d]pyrimidin-4-yl)(methyl)amino)-2-methylpyrrolidine-1-carboxylate (2 g, 4.65 mmol) and ((2R, 7aS)-2-fluorotetrahydro-1H-pyrrolizin-7a(5H)-yl)methanol (0.96 g, 6.04 mmol) were combined and dissolved in THE (23 mL). The soliton was cooled to 0° C. and LiHMDS solution (1.0 M in THF, 11.6 mL, 11.6 mmol) was added dropwise. The reaction mixture was allowed to warm to room temperature and was stirred for 24 h. The solution was concentrated, and the crude residue was directly purified by column chromatography (0→100% EtOAc with 5% Et₃N/hexanes) to provide the desired product (1.1 g, 2.0 mmol, 43% yield) as a 6:6:1:1 cis:cis:trans:trans mixture of diastereomers. (Cis diastereomers reported). LC/MS (ESI) m/z: [M+H]⁺ calcd for C₂₆H₃₆C₁F₂N₆O₃ 553.2; found 553.4; ¹H NMR (500 MHz, CDCl₃) δ 8.91 (s, 1H), 5.26 (d, J=55.0 Hz, 1H), 5.00-4.91 (m, 1H), 4.59-4.46 (m, 1H), 4.36-4.13 (m, 2H), 3.70-3.57 (m, 1H), 3.50 (s, 3H), 3.45-3.32 (m, 2H), 3.27-3.11 (m, 3H), 3.02-2.92 (m, 1H), 2.51-2.26 (m, 1H), 2.27-2.06 (m, 3H), 1.97-1.79 (m, 3H), 1.49-1.47 (m, 9H), 1.03 (br d, J=6.4 Hz, 3H).

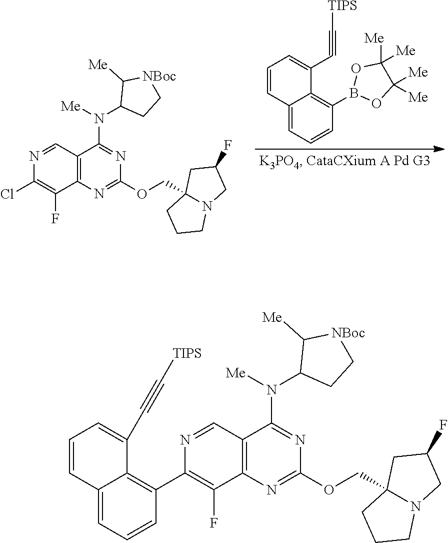

### Step 4: Preparation of tert-butyl 3-((8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)(methyl)amino)-2-methylpyrrolidine-1-carboxylate

tert-Butyl 3-((7-chloro-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidine-1-carboxylate (250 mg, 0.45 mmol), triisopropyl((8-(4,4,5,5-tetramethyl-1,3,2-dioxaborolan-2-yl)naphthalen-1-yl)ethynyl)silane (295 mg, 0.45 mmol), and methanesulfonato(diadamantyl-n-butylphosphino)-2′-amino-1,1′-biphenyl-2-yl)palladium(II) dichloromethane adduct, [cataCXium A Palladacycle Gen. 3] (3.3 mg, 4.5 μmol) were combined as solids in a microwave vial. The vial was sealed. The atmosphere was evacuated and replaced with nitrogen. This process was performed three times. Degassed dioxane (2.2 mL) and potassium phosphate solution (2.0 M in water, 680 μL, 1.36 mmol) were added, and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product. LCMS (ESI) m/z: [M+H]⁺ calcd for C₄₇H₆₃F₂N₆O₃Si 825.5; found 825.4.

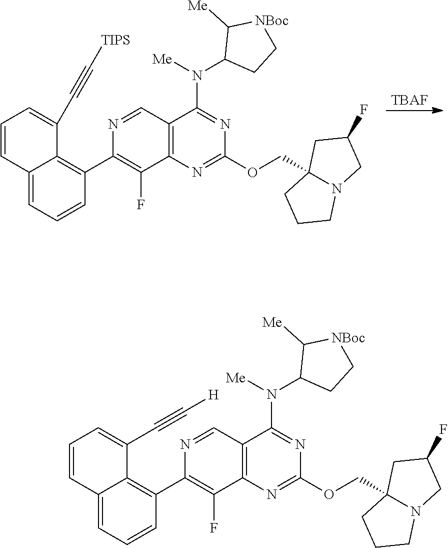

### Step 5: Preparation of tert-butyl 3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidine-1-carboxylate

tert-Butyl 3-((8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)(methyl)amino)-2-methylpyrrolidine-1-carboxylate (370 mg, 0.448 mmol) was dissolved in THE (4.5 mL) and the solution was cooled to 0° C. TBAF solution (1.0 M in THF, 540 μL, 0.540 mmol) was added dropwise, and the reaction mixture was warmed to room temperature. After 5 min, the reaction mixture was concentrated, and the crude residue was purified by column chromatography (50→100% EtOAc with 5% Et₃N/hexanes) to provide the desired product (270 mg, 0.404 mmol, 90% yield over two steps) as a 6:6:1:1 cis:cis:trans:trans mixture of diastereomers (cis diastereomers reported). LCMS (ESI) m/z: [M+H]⁺ calcd for C₃₈H₄₃F₂N₆O₃ 669.3; found 669.4; ¹H NMR (500 MHz, CDCl₃) δ 9.19-9.15 (m, 1H), 8.01-7.94 (m, 2H), 7.75 (br d, J=6.9 Hz, 1H), 7.65-7.56 (m, 2H), 7.46 (t, J=7.7 Hz, 1H), 0.00 (d, J=54.5 Hz, 1H), 5.10-4.89 (m, 1H), 4.66-4.52 (m, 1H), 4.38-4.15 (m, 2H), 3.66-3.60 (m, 1H), 3.59-3.54 (m, 3H), 3.43-3.38 (m, 1H), 3.29-3.20 (m, 2H), 3.18-3.15 (m, 1H), 3.00-2.94 (m, 1H), 2.57-2.53 (m, 1H), 2.43-2.33 (m, 1H), 2.29-2.09 (m, 4H), 1.98-1.85 (m, 3H), 1.50-1.48 (m, 9H), 1.10-1.05 (m, 3H).

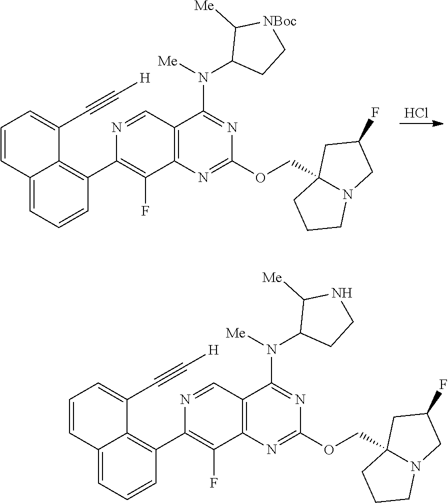 [SMILES: C#Cc1cccc2cccc(-c3ncc4c(N(C)C5CCN(C(=O)OC(C)(C)C)C5C)nc(OC[C@@]56CCCN5C[C@H](F)C6)nc4c3F)c12 ; Cl ; C#Cc1cccc2cccc(-c3ncc4c(N(C)C5CCNC5C)nc(OC[C@@]56CCCN5C[C@H](F)C6)nc4c3F)c12]

### Step 5: Preparation of 7-(8-ethynylnaphthalen-1-yl)-8-fluoro-2-(((2R, 7aS)-2-fluorotetrahydro-H-pyrrolizin-7a(5H)-yl)methoxy)-N-methyl-N-(2-methylpyrrolidin-3-yl)pyrido[4,3-d]pyrimidin-4-amine

Tert-Butyl 3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidine-1-carboxylate (270 mg, 0.404 mmol) was dissolved in MeCN (4.0 mL), and the solution was cooled to 0° C. HCl solution (4.0 M in dioxane, 1.0 mL, 4.0 mmol) was added dropwise. After 2 h, 1-methylimidazole (500 μL) was added, and the reaction mixture was concentrated. The crude residue was used directly in the next step, without additional purification (quantitative yield assumed). LCMS (ESI) m/z: [M+H]⁺ calcd for C₃₃H₃₅F₂N₆O 569.3; found 569.3.

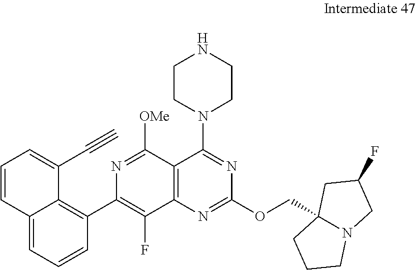 [SMILES: C#Cc1cccc2cccc(-c3nc(OC)c4c(N5CCNCC5)nc(OC[C@@]56CCCN5C[C@H](F)C6)nc4c3F)c12]

### 7-(8-ethynylnaphthalen-1-yl)-8-fluoro-2-(((2R, 7aS)-2-fluorotetrahydro-1H-pyrrolizin-7a(5H)-yl)methoxy)-5-methoxy-4-(piperazin-1-yl)pyrido[4,3-d]pyrimidine

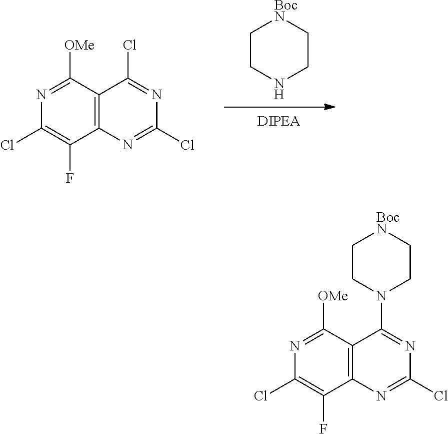 [SMILES: COc1nc(Cl)c(F)c2nc(Cl)nc(Cl)c12 ; CC(C)(C)OC(=O)N1CCNCC1 ; COc1nc(Cl)c(F)c2nc(Cl)nc(N3CCN(C(=O)OC(C)(C)C)CC3)c12]

### Step 1: Preparation of tert-butyl 4-(2,7-dichloro-8-fluoro-5-methoxypyrido[4,3-d]pyrimidin-4-yl)piperazine-1-carboxylate

2,4,7-Trichloro-8-fluoro-5-methoxypyrido[4,3-d]pyrimidine (500 mg, 1.770 mmol) [see CN115304623] was dissolved in DCM (9.0 mL), and the solution was cooled to −10° C. (wet ice/acetone). DIPEA (0.46 mL, 2.7 mmol) was added dropwise, followed by tert-butyl piperazine-1-carboxylate (330 mg, 1.77 mmol) as a solid. The reaction mixture was allowed to warm to room temperature and was stirred for 5 min. The reaction mixture was concentrated, and the crude residue was used directly in the next step, without additional purification (quantitative yield assumed). LCMS (ESI) m/z: [M+H]⁺ calcd for C₁₇H₂₁C₁₂FN₅O₃ 432.1; found 432.1; ¹H NMR (500 MHz, CDCl₃) δ 4.09 (s, 3H), 3.75-3.66 (m, 4H), 3.61-3.56 (m, 4H), 1.48 (s, 9H).

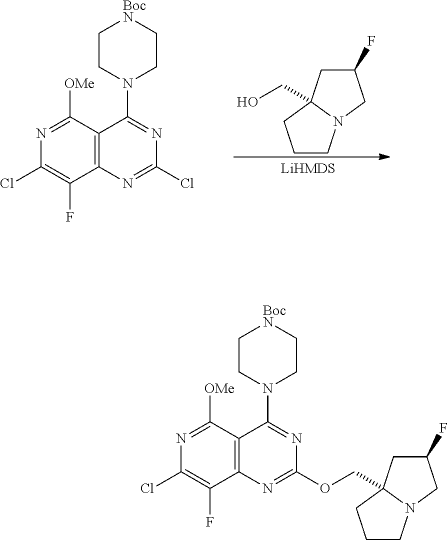 [SMILES: COc1nc(Cl)c(F)c2nc(Cl)nc(N3CCN(C(=O)OC(C)(C)C)CC3)c12 ; OC[C@@]12CCCN1C[C@H](F)C2 ; COc1nc(Cl)c(F)c2nc(OC[C@@]34CCCN3C[C@H](F)C4)nc(N3CCN(C(=O)OC(C)(C)C)CC3)c12]

### Step 2: Preparation of tert-butyl 4-(7-chloro-8-fluoro-2-(((2R, 7aS)-2-fluorotetrahydro-1H-pyrrolizin-7a(5H)-yl)methoxy)-5-methoxypyrido[4,3-d]pyrimidin-4-yl)piperazine-1-carboxylate

tert-Butyl 4-(2,7-dichloro-8-fluoro-5-methoxypyrido[4,3-d]pyrimidin-4-yl)piperazine-1-carboxylate (735 mg, 1.70 mmol) was suspended in THE (8.5 mL), and ((2R, 7aS)-2-fluorotetrahydro-1H-pyrrolizin-7a(5H)-yl)methanol (406 mg, 2.55 mmol) was added. The mixture was cooled to 0° C., and LiHMDS solution (1.0 M in THF, 3.7 mL, 3.7 mmol) was added dropwise. Following addition, the reaction mixture was allowed to warm to room temperature and was stirred for 24 h. The solution was concentrated, and the crude residue was purified by column chromatography (0→100% EtOAc with 5% Et₃N/hexanes) to provide the desired product (850 mg, 1.53 mmol, 90% yield) as a pale yellow solid. LC/MS (ESI) m/z: [M+H]⁺ calcd for C₂₅H₃₄C₁F₂N₆O₃ 555.2; found 555.1; ¹H NMR (500 MHz, CDCl₃) δ 5.23 (d, J=53.0 Hz, 1H), 4.20 (d, J=10.4 Hz, 1H), 4.08 (d, J=10.4 Hz, 1H), 4.04 (s, 3H), 3.63-3.58 (m, 4H), 3.57-3.52 (m, 4H), 3.25-3.07 (m, 3H), 2.94 (s, 1H), 2.27-2.07 (m, 3H), 1.97-1.80 (m, 3H), 1.47 (s, 9H).

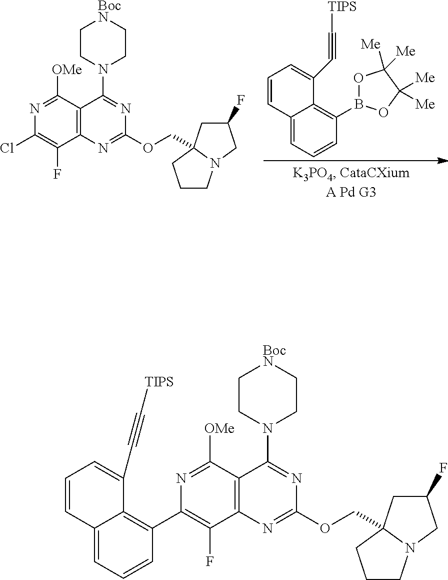

### Step 3: Preparation of tert-butyl 4-(8-fluoro-2-(((2R, 7aS)-2-fluorotetrahydro-1H-pyrrolizin-7a(5H)-yl)methoxy)-5-methoxy-7-(8-((triisopropylsilyl)ethynyl)naphthalen-1-yl)pyrido[4,3-d]pyrimidin-4-yl)piperazine-1-carboxylate

tert-Butyl 4-(7-chloro-8-fluoro-2-(((2R, 7aS)-2-fluorotetrahydro-1H-pyrrolizin-7a(5H)-yl)methoxy)-5-methoxypyrido[4,3-d]pyrimidin-4-yl)piperazine-1-carboxylate (50 mg, 0.090 mmol), triisopropyl((8-(4,4,5,5-tetramethyl-1,3,2-dioxaborolan-2-yl)naphthalen-1-yl)ethynyl)silane (39 mg, 0.09 mmol), and methanesulfonato(diadamantyl-n-butylphosphino)-2′-amino-1,1′-biphenyl-2-yl)palladium(II) dichloromethane adduct, [cataCXium A Palladacycle Gen. 3] (3.3 mg, 4.5 μmol) were combined as solids in a microwave vial. The vial was sealed. The atmosphere was evacuated and replaced with nitrogen. This process was performed three times. Degassed dioxane (0.45 mL) and potassium phosphate solution (2.0 M in water, 130 μL, 0.27 mmol) were added, and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product (74 mg, 0.089 mmol, 99% yield). LC/MS (ESI) m/z: [M+H]⁺ calcd for C₄₆H₆₁F₂N₆O₄Si 827.4; found 827.9; ¹H NMR (500 MHz, CDCl₃) δ 7.95-7.89 (m, 2H), 7.80 (d, J=7.2 Hz, 1H), 7.61-7.54 (m, 2H), 7.47-7.43 (m, 1H), 5.24 (d, J=54.1 Hz, 1H), 4.24 (dd, J=10.5, 0.8 Hz, 1H), 4.10 (t, J=9.8 Hz, 1H), 4.00 (s, 3H), 3.73-3.62 (m, 4H), 3.62-3.51 (m, 4H), 3.21 (s, 3H), 3.00-2.92 (m, 1H), 2.33-2.10 (m, 3H), 1.98-1.81 (m, 3H), 1.50 (s, 9H), 0.88-0.85 (m, 18H), 0.58 (spt, J=7.4 Hz, 3H).

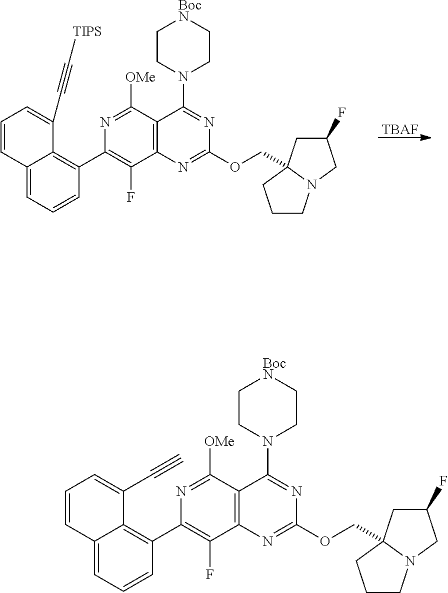

### Step 4: Preparation of tert-butyl 4-(7-(8-ethynylnaphthalen-1-yl)-8-fluoro-2-(((2R, 7aS)-2-fluorotetrahydro-1H-pyrrolizin-7a(5H)-yl)methoxy)-5-methoxypyrido[4,3-d]pyrimidin-4-yl)piperazine-1-carboxylate

tert-Butyl 4-(8-fluoro-2-(((2R, 7aS)-2-fluorotetrahydro-1H-pyrrolizin-7a(5H)-yl)methoxy)-5-methoxy-7-(8-((triisopropylsilyl)ethynyl)naphthalen-1-yl)pyrido[4,3-d]pyrimidin-4-yl)piperazine-1-carboxylate (74 mg, 0.089 mmol) was dissolved in THE (0.9 mL), and the solution was cooled to 0° C. TBAF solution (1.0 M in THF, 89 μL, 0.089 mmol) was added dropwise, and the reaction mixture was warmed to room temperature. After 5 min, the reaction mixture was concentrated, and the crude residue was purified by column chromatography (50→100% EtOAc with 5% Et₃N/hexanes) to provide the desired product (54 mg, 0.081 mmol, 90% yield). LC/MS (ESI) m/z: [M+H]⁺ calcd for C₃₇H₄₁F₂N₆O₄ 671.3; found 671.6; ¹H NMR (500 MHz, CDCl₃) δ 7.99-7.92 (m, 2H), 7.75 (dd, J=7.2, 1.3 Hz, 1H), 7.63-7.58 (m, 2H), 7.45 (dd, J=8.1, 7.3 Hz, 1H), 5.25 (d, J=54.0 Hz, 1H), 4.28-4.23 (m, 1H), 4.16-4.11 (m, 1H), 3.99 (s, 3H), 3.70-3.64 (m, 4H), 3.64-3.59 (m, 4H), 3.28-3.12 (m, 3H), 3.00-2.92 (m, 1H), 2.61-2.58 (m, 1H), 2.30-2.11 (m, 3H), 1.90 (s, 3H), 1.50 (s, 9H).

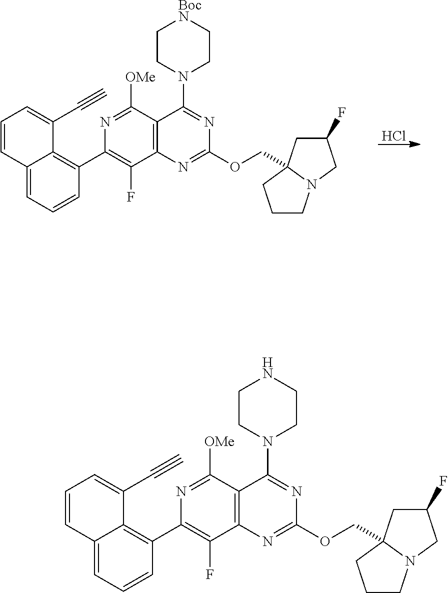 [SMILES: C#Cc1cccc2cccc(-c3nc(OC)c4c(N5CCN(C(=O)OC(C)(C)C)CC5)nc(OC[C@@]56CCCN5C[C@H](F)C6)nc4c3F)c12 ; Cl ; C#Cc1cccc2cccc(-c3nc(OC)c4c(N5CCNCC5)nc(OC[C@@]56CCCN5C[C@H](F)C6)nc4c3F)c12]

### Step 5: Preparation of 7-(8-ethynylnaphthalen-1-yl)-8-fluoro-2-(((2R, 7aS)-2-fluorotetrahydro-1H-pyrrolizin-7a(5H)-yl)methoxy)-5-methoxy-4-(piperazin-1-yl)pyrido[4,3-d]pyrimidine

tert-Butyl 4-(7-(8-ethynylnaphthalen-1-yl)-8-fluoro-2-(((2R, 7aS)-2-fluorotetrahydro-1H-pyrrolizin-7a(5H)-yl)methoxy)-5-methoxypyrido[4,3-d]pyrimidin-4-yl)piperazine-1-carboxylate (54 mg, 0.081 mmol) was dissolved in MeCN (0.8 mL), and the solution was cooled to 0° C. HCl solution (4.0 M in dioxane, 200 μL, 0.8 mmol) was added dropwise, and the reaction mixture was allowed to warm to room temperature. After 1.5 h, 1-methylimidazole (100 μL) was added, and the solution was concentrated. The crude residue was used directly in the next step, without additional purification. LC/MS (ESI) m/z: [M+H]⁺ calcd for C₃₂H₃₃F₂N₆O₂ 571.3; found 571.4.

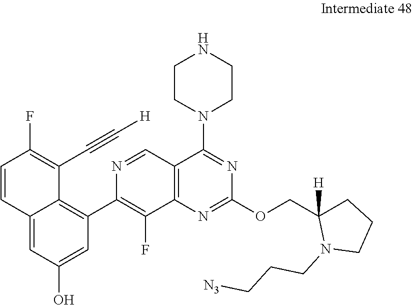 [SMILES: C#Cc1c(F)ccc2cc(O)cc(-c3ncc4c(N5CCNCC5)nc(OC[C@@H]5CCCN5CCCN=[N+]=[N-])nc4c3F)c12]

### (S)-4-(2-((1-(3-azidopropyl)pyrrolidin-2-yl)methoxy)-8-fluoro-4-(piperazin-1-yl)pyrido[4,3-d]pyrimidin-7-yl)-5-ethynyl-6-fluoronaphthalen-2-ol

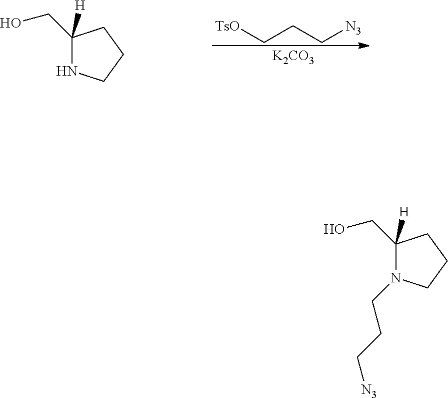

### Step 1: Preparation of (S)-(1-(3-azidopropyl)pyrrolidin-2-yl)methanol

(S)-Pyrrolidin-2-ylmethanol (350 mg, 3.46 mmol) was dissolved in MeCN (3.5 mkL), and potassium carbonate (1.4 g, 10.4 mmol) was added followed by 3-azidopropyl 4-methylbenzenesulfonate (972 mg, 3.81 mmol). The reaction mixture was stirred at room temperature for 20 h. The mixture was filtered through a pad of diatomaceous earth (Celite®) and was concentrated. The crude residue was shaken up in EtOAc (100 mL) and was extracted with HCl (3×50 mL). The combined aqueous layers were neutralized with NaOH solution (3 M, 50 mL), and the solution was lyophilized. EtOAc (50 mL) was added to the crude residue, and the solution was filtered and concentrated to provide the desired product (450 mg, 2.4 mmol, 71% yield). ¹H NMR (600 MHz, CDCl₃) δ 3.65-3.57 (m, 1H), 3.41-3.30 (m, 3H), 3.20-3.11 (m, 1H), 2.87-2.77 (m, 1H), 2.61-2.54 (m, 1H), 2.58 (br s, 1H), 2.39-2.29 (m, 1H), 2.27-2.18 (m, 1H), 1.92-1.82 (m, 1H), 1.81-1.68 (m,

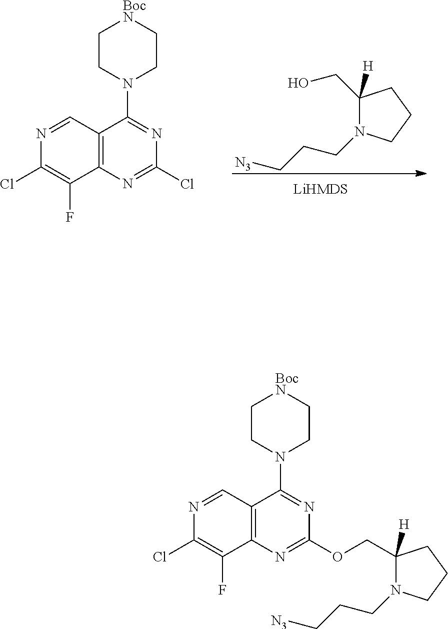 [SMILES: CC(C)(C)OC(=O)N1CCN(c2nc(Cl)nc3c(F)c(Cl)ncc23)CC1 ; [N-]=[N+]=NCCCN1CCC[C@H]1CO ; CC(C)(C)OC(=O)N1CCN(c2nc(OC[C@@H]3CCCN3CCCN=[N+]=[N-])nc3c(F)c(Cl)ncc23)CC1]

### Step 2: Preparation of tert-butyl (S)-4-(2-((1-(3-azidopropyl)pyrrolidin-2-yl)methoxy)-7-chloro-8-fluoropyrido[4,3-d]pyrimidin-4-yl)piperazine-1-carboxylate

tert-butyl 4-(2,7-dichloro-8-fluoropyrido[4,3-d]pyrimidin-4-yl)piperazine-1-carboxylate (400 mg, 0.994 mmol) was suspended in THF (5.0 mL), and (S)-(1-(3-azidopropyl)pyrrolidin-2-yl)methanol (183 mg, 0.994 mmol) was added. The mixture was cooled to 0° C., and LiHMDS solution (1.0 M in THF, 2.2 mL, 2.2 mmol) was added dropwise. Following addition, the reaction mixture was allowed to warm to room temperature and was stirred for 24 h. The solution was concentrated, and the crude residue was purified by column chromatography (0→100% EtOAc with 5% Et₃N/hexanes) to provide the desired product (340 mg, 0.62 mmol, 62% yield). LC/MS (ESI) m/z: [M+H]* calcd for C₂₄H₃₄C₁FN₉O₃ 550.2; found 550.3.

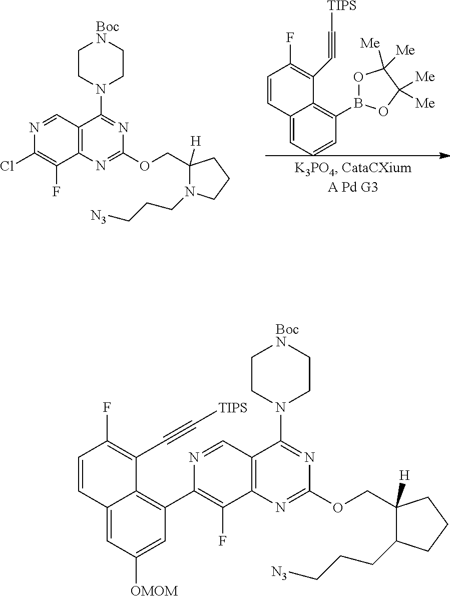

### Step 3: Preparation of tert-butyl (S)-4-(2-((1-(3-azidopropyl)pyrrolidin-2-yl)methoxy)-8-fluoro-7-(7-fluoro-3-(methoxymethoxy)-8-((triisopropylsilyl)ethynyl)naphthalen-1-yl)pyrido[4,3-d]pyrimidin-4-yl)piperazine-1-carboxylate

tert-Butyl (S)-4-(2-((1-(3-azidopropyl)pyrrolidin-2-yl)methoxy)-7-chloro-8-fluoropyrido[4,3-d]pyrimidin-4-yl)piperazine-1-carboxylate (340 mg, 0.618 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (475 mg, 0.927 mmol), and methanesulfonato(diadamantyl-n-butylphosphino)-2′-amino-1,1′-biphenyl-2-yl)palladium(II) dichloromethane adduct, [cataCXium A Palladacycle Gen. 3] (4.5 mg, 6.2 μmol)were combined as solids in a microwave vial. The vial was sealed. The atmosphere was evacuated and replaced with nitrogen. This process was performed three times. Degassed dioxane (3.1 mL) and potassium phosphate solution (2.0 M in water, 930 μL, 1.85 mmol) were added, and the reaction mixture was heated at 100° C. in the microwave for 1.5 h. The reaction mixture was directly concentrated, and the crude residue was purified by column chromatography (20→100% EtOAc with 5% Et₃N/hexanes) to provide the desired product. LCMS (ESI) m/z: [M+H]⁺ calcd for C₄₇H₆₄F₂N₉O₅Si 900.5; found 900.5.

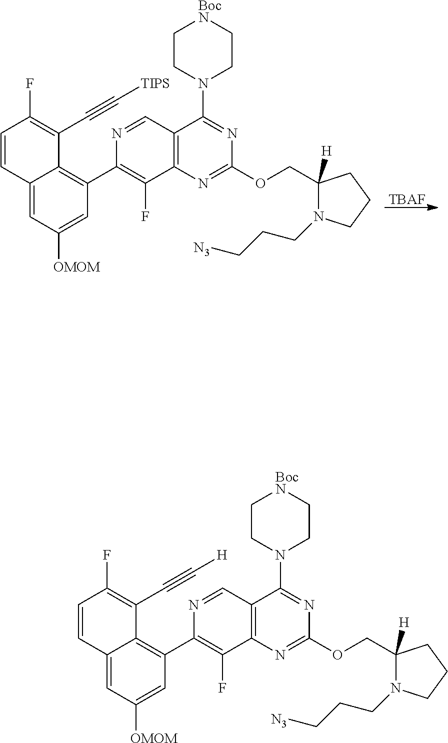

### Step 4: Preparation of tert-butyl (S)-4-(2-((1-(3-azidopropyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)piperazine-1-carboxylate

tert-Butyl (S)-4-(2-((1-(3-azidopropyl)pyrrolidin-2-yl)methoxy)-8-fluoro-7-(7-fluoro-3-(methoxymethoxy)-8-((triisopropylsilyl)ethynyl)naphthalen-1-yl)pyrido[4,3-d]pyrimidin-4-yl)piperazine-1-carboxylate (550 mg, 0.61 mmol) was dissolved in THF (6.1 mL), and the solution was cooled to 0° C. TBAF solution (1.0 M in THF, 610 μL, 0.61 mmol) was added dropwise, and the reaction mixture was warmed to room temperature. After 5 min, the reaction mixture was concentrated, and the crude residue was purified by column chromatography (50→100% EtOAc with 5% Et₃N/hexanes) to provide the desired product (400 mg, 0.54 mmol, 88% yield). LC/MS (ESI) m/z: [M+H]⁺ calcd for C₃₈H₄₄F₂N₉O₅ 744.3; found 744.3.

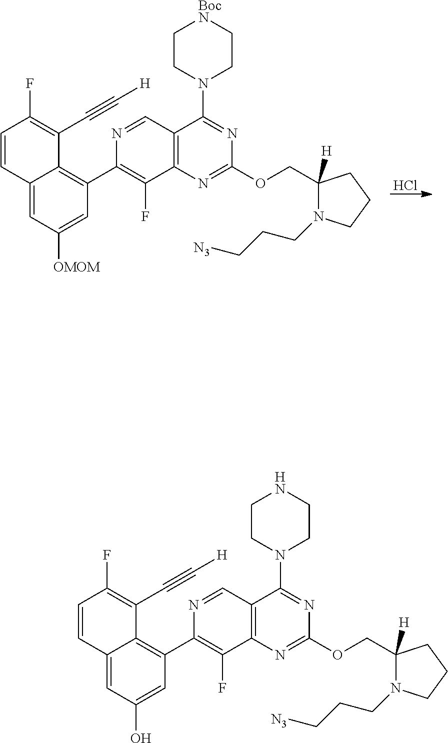

### Step 5: Preparation of(S)-4-(2-((1-(3-azidopropyl)pyrrolidin-2-yl)methoxy)-8-fluoro-4-(piperazin-1-yl)pyrido[4,3-d]pyrimidin-7-yl)-5-ethynyl-6-fluoronaphthalen-2-ol

tert-Butyl (S)-4-(2-((1-(3-azidopropyl)pyrrolidin-2-yl)methoxy)-7-(8-ethynyl-7-fluoro-3-(methoxymethoxy)naphthalen-1-yl)-8-fluoropyrido[4,3-d]pyrimidin-4-yl)piperazine-1-carboxylate (400 mg, 0.538 mmol) was dissolved in MecN (5.4 mL), and the solution was cooled to 0° C. HCl solution (4.0 M in dioxane, 1.3 mL, 5.4 mmol) was added dropwise, and the reaction mixture was allowed to warm to room temperature. After 1 h, 1-methylimidazole (1 mL) was added, and the solution was concentrated. The crude residue was used directly in the next step, without additional purification. LCMS (ESI) m/z: [M+H]⁺ calcd for C₃₁H₃₂F₂N₉O₂ 600.3; found 600.2.

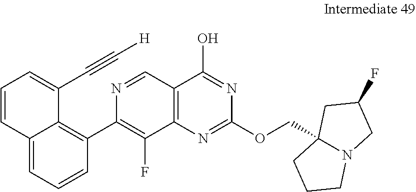 [SMILES: C#Cc1cccc2cccc(-c3ncc4c(O)nc(OC[C@@]56CCCN5C[C@H](F)C6)nc4c3F)c12]

### 7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-ol

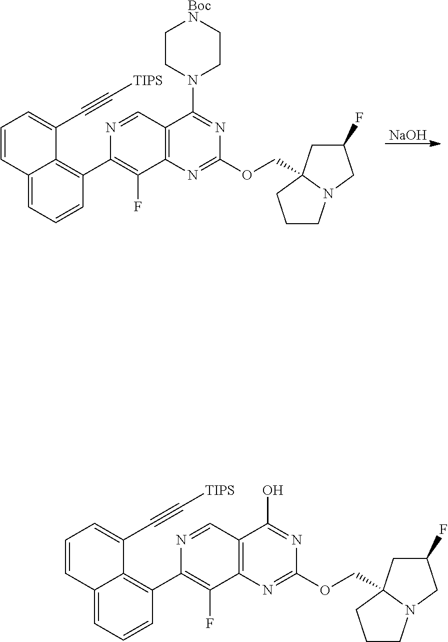

### Step 1: Preparation of 8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-ol

tert-Butyl 4-(8-fluoro-2-(((2R, 7aS)-2-fluorotetrahydro-1H-pyrrolizin-7a(5H)-yl)methoxy)-7-(8-((triisopropylsilyl)ethynyl)naphthalen-1-1)pyrido[4,3-d]pyrimidin-4-yl)piperazine-1-carboxylate (1.2 g, 1.505 mmol) was dissolved in ethanol (10 mL), and the solution was cooled to 0° C. Aqueous sodium hydroxide solution (1.0 M, 4.5 mL, 4.5 mmol) was added followed by MeOH (one drop, ˜20 μL). The resulting reaction mixture was heated at 70° C. for 3 h. The reaction mixture was quenched with ice cold water (20 mL) and ethyl acetate (50 mL) was added. The layers were separated and the aqueous phase was further extracted with EtOAc (2×50 mL). The combined organic layers were dried over sodium sulfate, filtered, and concentrated. The crude residue (900 mg) was used directly in the next step, without additional purification. LC/MS (ESI) m/z: [M+H]⁺ calcd for C₃₆H₄₃F₂N₄O₂ Si 629.3; found 629.0.

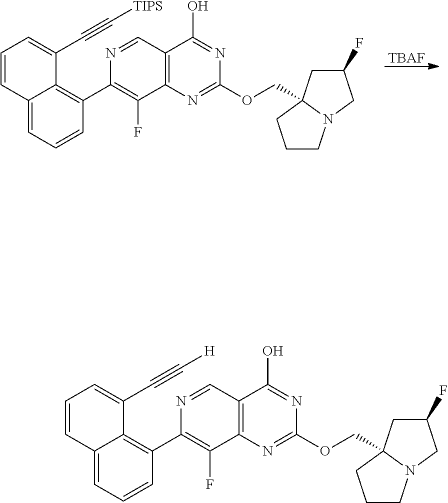

### Step 2: Preparation of 7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-ol

8-Fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-ol (900 mg, 1.43 mmol) was dissolved in THE (20 mL), and the solution was cooled to 0° C. TBAF solution (1.0 M in THF, 2.9 mL, 2.9 mmol) was added dropwise, and the resulting reaction mixture was stirred at 0° C. for 2 h. After the completion, the reaction mixture was partitioned between EtOAc (50 mL) and water (50 mL). The layers were separated, and the aqueous phase was further extracted with EtOAc (2×30 mL). The combined organic layers were dried over sodium sulfate, filtered, and concentrated. The crude residue was purified by column chromatography to provide the desired product (400 mg, 0.84 mmol, 56% yield over two steps). LC/MS (ESI) m/z: [M+H]⁺ calcd for C₂₇H₂₃F₂N₄O₂ 473.2; found 473.0.

General Scheme for the preparation of the following two intermediates:

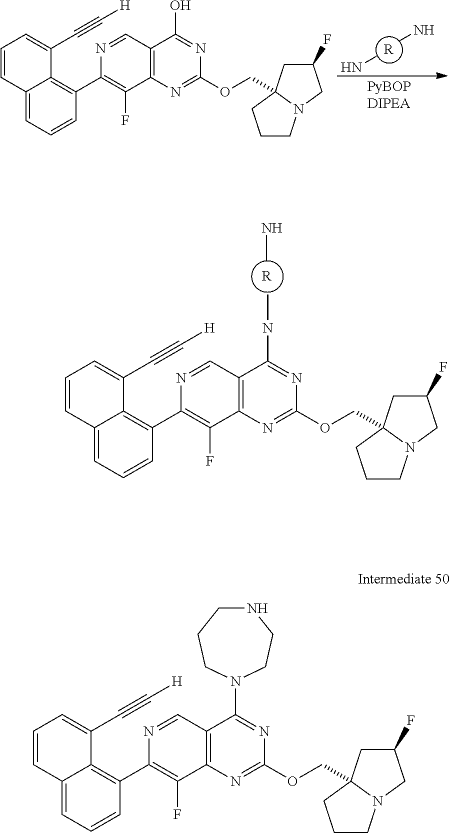

### 4-(1,4-diazepan-1-yl)-7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidine

7-(8-Ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-ol (100 mg, 0.212 mmol) was dissolved in MeCN (5 mL), and 1,4-diazepane (21 mg, 0.21 mmol) was added followed by DIPEA (0.11 mL, 0.64 mmol) and PyBOP (220 mg, 0.42 mmol). The resulting reaction mixture was heated at 60° C. for 1 h. The solution was concentrated, and the crude residue was directly purified by reverse phase column chromatography [Redisep 40 gm C_{18, 20}-40 micron, THF:Water:ACN (50:20:30) as a diluent and 72% Methanol in 10 mM Ammonium bicarbonate as an eluent] to provide the desired product (14 mg, 12% yield) as an off white solid. LC/MS (ESI) m/z: [M+H]⁺ calcd for C₃₂H₃₃F₂N₆O 555.3; found 555.2; ¹H NMR (400 MHz, DMSO-d₆): δ 9.10 (s, 1H), 8.17-8.12 (m, 2H), 7.74-7.68 (m, 2H), 7.60-7.55 (m, 2H), 5.28 (d, J=53.60 Hz, 1H), 4.07-4.02 (m, 6H), 3.71 (d, J=1.20 Hz, 1H), 3.35-3.02 (m, 6H), 2.87-2.83 (m, 3H), 2.05-1.77 (m, 8H).

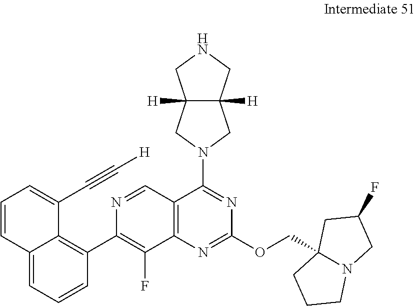 [SMILES: C#Cc1cccc2cccc(-c3ncc4c(N5C[C@H]6CNC[C@H]6C5)nc(OC[C@@]56CCCN5C[C@H](F)C6)nc4c3F)c12]

### 7-(8-ethynylnaphthalen-1-yl)-8-fluoro-2-(((2R, 7aS)-2-fluorotetrahydro-1H-pyrrolizin-7a(5H)-yl)methoxy)-4-((3aR, 6aS)-hexahydropyrrolo[3,4-c]pyrrol-2(1H)-yl)pyrido[4,3-d]pyrimidine

This intermediate was prepared following the general procedure above using (3as,6as)-octahydropyrrolo[3,4-c]pyrrole in place of 1,4-diazepane. LCMS (ESI) m/z: [M+H]⁺ calcd for C₃₃H₃₃F₂N₆O 567.3; found 567.2; ¹H NMR (400 MHz, DMSO-d₆): 1H-NMR (400 MHz, DMSO-d₆): δ 9.22 (s, 1H), 8.15 (t, J=8.00 Hz, 2H), 7.75-7.69 (m, 2H), 7.61-7.57 (m, 2H), 5.29 (d, J=53.60 Hz, 1H), 4.15-3.78 (m, 4H), 3.61 (s, 1H), 3.11-3.02 (m, 5H), 2.71-2.70 (m, 3H), 2.14-2.06 (m, 3H), 1.78-1.76 (m, 3H), 1.35-1.31 (m, 3H), 0.93-0.88 (m, 2H).

General Scheme for the preparation of the following intermediates:

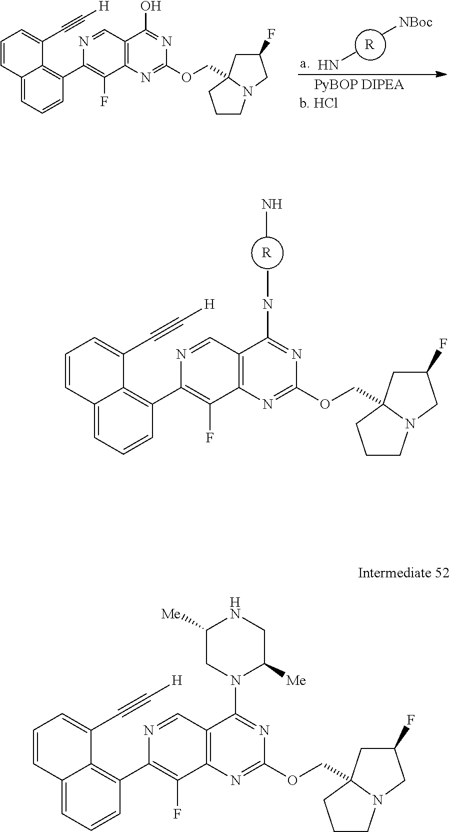

### 4-((2R, 5S)-2,5-dimethylpiperazin-1-yl)-7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidine

### Step 1: Preparation of tert-butyl (2S,5R)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-2,5-dimethylpiperazine-1-carboxylate

7-(8-Ethynylnaphthalen-1-yl)-8-fluoro-2-(((2R, 7aS)-2-fluorotetrahydro-1H-pyrrolizin-7a(5H)-yl)mehoxy)pyrido[4,3-d]pyrimidin-4-ol (160 mg, 0.34 mmol) was dissolved in MeCN (8 mL), and DIPEA (0.18 mL, 1.02 mmol) and tert-butyl (2S,5R)-2,5-dimethylpiperazine-1-carboxylate (72 mg, 0.34 mmol) were added, followed by PyBOP (350 mg, 0.68 mmol). The reaction mixture was heated at 60° C. for 1 h. The solution was concentrated, and the crude residue was triturated with n-pentane (2×20 mL) and dried to provide the desired product (200 mg) as brown solid, which was used directly in the next step, without additional purification. LCMS (ESI) m/z: [M+H]⁺ calcd for C₃₈H₄₃F₂N₆O₃ 669.3; found 669.2.

### Step 2: Preparation of 4-((2R, 5S)-2,5-dimethylpiperazin-1-yl)-7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidine

tert-Butyl (2S,5R)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-2,5-dimethylpiperazine-1-carboxylate (200 mg, 0.299 mmol) was dissolved in MeCN (4 mL), and the solution was cooled to 0° C. HCl solution (4.0 M in dioxane, 0.75 mL, 3.0 mmol) was add was added dropwise, and the reaction mixture was allowed to warm to room temperature. After 2 h, the reaction mixture was concentrated, and the crude residue was purified by reverse phase column chromatography [Redisep 50 gm C18, 20-40 micron, THF:Water:ACN (50:20:30) as a diluent and 20% ACN in 5 mM Ammonium formate as an eluent] to provide the desired product as its formic acid salt. The material was partitioned between EtOAc (20 mL) and a saturated aqueous solution of NaHCO₃(10 mL). The layers were separated, and the organic phase was dried over sodium sulfate, filtered, and concentrated. The residue was lyophilized to provide the desired product (18 mg, 0.032 mmol, 9% yield over two steps) as a brown solid. LC/MS (ESI) m/z: [M+H]⁺ calcd for C₃₃H₃₅F₂N₆O 569.3; found 569.3; ¹H-NMR (400 MHz, DMSO-d₆): δ 9.04-8.98 (m, 1H), 8.20-8.16 (m, 2H), 7.86-7.70 (m, 2H), 7.64-7.56 (m, 2H), 5.28 (d, J=56.00 Hz, 1H), 4.12-3.59 (m, 4H), 3.10-3.01 (m, 4H), 2.35-1.72 (m, 9H), 1.44-0.88 (m, 9H).

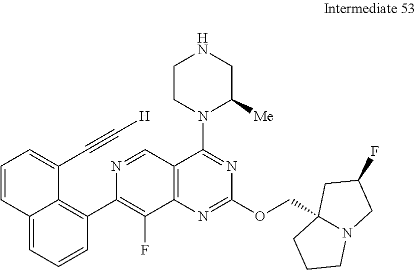 [SMILES: C#Cc1cccc2cccc(-c3ncc4c(N5CCNC[C@H]5C)nc(OC[C@@]56CCCN5C[C@H](F)C6)nc4c3F)c12]

### 7-(8-ethynylnaphthalen-1-yl)-8-fluoro-2-(((2R, 7aS)-2-fluorotetrahydro-1H-pyrrolizin-7a(5H)-yl)methoxy)-4-((R)-2-methylpiperazin-1-yl)pyrido[4,3-d]pyrimidine

This intermediate was prepared following the general procedure above using tert-butyl (R)-3-methylpiperazine-1-carboxylate in place of tert-butyl (2S,5R)-2,5-dimethylpiperazine-1-carboxylate. LCMS (ESI) m/z: [M+H]⁺ calcd for C₃₂H₃₃F₂N₆O 555.3; found 555.2; ¹H NMR (400 MHz, DMSO-d₆): δ 9.04-8.97 (m, 1H), 8.15 (t, J=8.40 Hz, 1H), 7.74-7.68 (m, 1H), 7.62-7.58 (m, 1H), 5.28 (d, J=56.00 Hz, 1H), 4.11-3.87 (m, 1H), 3.71-3.69 (m, 1H), 3.49-3.42 (m, 1H), 3.31-3.00 (m, 7H), 2.99-2.84 (m, 2H), 2.13-1.77 (m, 2H), 1.60-1.47 (m, 3H), 1.35-1.29 (m, 4H), 0.96-0.92 (m, 6H).

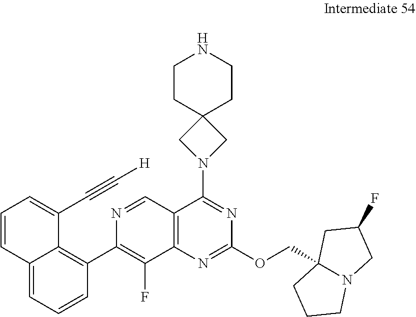 [SMILES: C#Cc1cccc2cccc(-c3ncc4c(N5CC6(CCNCC6)C5)nc(OC[C@@]56CCCN5C[C@H](F)C6)nc4c3F)c12]

### 7-(8-ethynylnaphthalen-1-yl)-8-fluoro-2-(((2R, 7aS)-2-fluorotetrahydro-1H-pyrrolizin-7a(5H)-yl)methoxy)-4-(2,7-diazaspiro[3.5]nonan-2-yl)pyrido[4,3-d]pyrimidine

This intermediate was prepared following the general procedure above using tert-butyl 2,7-diazaspiro[3.5]nonane-7-carboxylate in place of tert-butyl (2S,5R)-2,5-dimethylpiperazine-1-carboxylate. LCMS (ESI) m/z: [M+H]⁺ calcd for C₃₄H₃₅F₂N₆O 581.3; found 581.2; ¹H NMR (400 MHz, DMSO-d₆): δ 8.95 (s, 1H), 8.14 (dt, J=0.80, 11.07 Hz, 1H), 7.72-7.67 (m, 1H), 7.56 (dd, J=7.60, Hz, 1H), 5.27 (d, J=54.40 Hz, 1H), 4.50 (s, 2H), 4.11-3.98 (m, 5H), 3.71 (s, 1H), 3.31-3.01 (m, 3H), 2.83-2.82 (m, 1H), 2.69-2.55 (m, 5H), 2.12-2.11 (m, 1H), 2.04-2.03 (m, 1H), 1.85-1.73 (m, 10H).

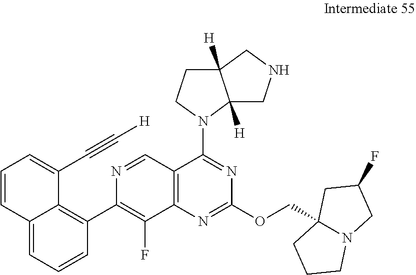 [SMILES: C#Cc1cccc2cccc(-c3ncc4c(N5CC[C@@H]6CNC[C@@H]65)nc(OC[C@@]56CCCN5C[C@H](F)C6)nc4c3F)c12]

### 7-(8-Ethynylnaphthalen-1-yl)-8-fluoro-2-(((2R, 7aS)-2-fluorotetrahydro-1H-pyrrolizin-7a(5H)-yl)methoxy)-4-((3aR, 6aR)-hexahydropyrrolo[3,4-b]pyrrol-1(2H)-yl)pyrido[4,3-d]pyrimidine

This intermediate was prepared following the general procedure above using tert-butyl (3aR, 6aR)-hexahydropyrrolo[3,4-b]pyrrole-5(1H)-carboxylate in place of tert-butyl (2S,5R)-2,5-dimethylpiperazine-1-carboxylate. LCMS (ESI) m/z: [M+H]⁺ calcd for C₃₃H₃₃F₂N₆O 567.3; found 567.2; ¹H NMR (400 MHz, DMSO-d₆): δ 9.21-9.18 (m, 1H), 8.15 (t, J=8.00 Hz, 1H), 7.75-7.68 (m, 2H), 7.62-7.58 (m, 2H), 5.28 (d, J=56.00 Hz, 1H), 4.9-5.0 (m, 1H), 4.17-4.04 (m, 4H), 3.75-3.66 (m, 1H), 3.13-2.83 (m, 8H), 2.07-1.78 (m, 8H), 1.35-1.24 (m, 2H).

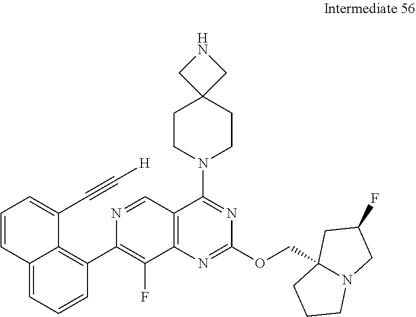 [SMILES: C#Cc1cccc2cccc(-c3ncc4c(N5CCC6(CC5)CNC6)nc(OC[C@@]56CCCN5C[C@H](F)C6)nc4c3F)c12]

### 7-(8-ethynylnaphthalen-1-yl)-8-fluoro-2-(((2R, 7aS)-2-fluorotetrahydro-1H-pyrrolizin-7a(5H)-yl)methoxy)-4-(2,7-diazaspiro[3.5]nonan-7-yl)pyrido[4,3-d]pyrimidine

This intermediate was prepared following the general procedure above using tert-butyl 2,7-diazaspiro[3.5]nonane-2-carboxylate in place of tert-butyl (2S,5R)-2,5-dimethylpiperazine-1-carboxylate. LCMS (ESI) m/z: [M+H]⁺ calcd for C₃₄H₃₅F₂N₆O 581.3; found 581.2; ¹H NMR (400 MHz, DMSO-d₆): δ 9.00 (s, 1H), 8.17-8.13 (m, 2H), 7.73-7.67 (m, 2H), 7.62-7.55 (m, 2H), 5.28 (d, J=56.00 Hz, 1H), 4.05-4.03 (m, 1H), 3.84 (s, 2H), 3.70 (s, 1H), 3.36-3.33 (m, 2H), 3.08-3.00 (m, 4H), 2.13-1.94 (m, 10H), 1.34-1.24 (m, 3H), 0.88-0.87 (m, 3H).

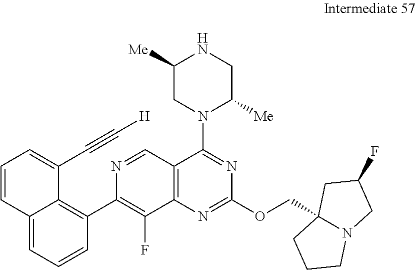 [SMILES: C#Cc1cccc2cccc(-c3ncc4c(N5C[C@@H](C)NC[C@@H]5C)nc(OC[C@@]56CCCN5C[C@H](F)C6)nc4c3F)c12]

### 4-((2S,5R)-2,5-dimethylpiperazin-1-yl)-7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidine

This intermediate was prepared following the general procedure above using tert-butyl (2R, 5S)-2,5-dimethylpiperazine-1-carboxylate in place of tert-butyl (2S,5R)-2,5-dimethylpiperazine-1-carboxylate, and BOP in place of PyBOP. LCMS (ESI) m/z: [M+H]* calcd for C₃₃H₃₅F₂N₆O 569.3; found 569.3; ¹H NMR (400 MHz, DMSO-d₆): δ 9.08 (d, J=21.20 Hz, 1H), 8.16 (t, J=8.40 Hz, 2H), 7.71-7.74 (m, 2H), 7.56-7.62 (m, 2H), 5.30 (d, J=52.00 Hz, 1H), 4.63 (m, 1H), 4.07-4.18 (m, 3H), 3.73-3.78 (m, 1H), 3.43-3.58 (m, 3H), 2.52-3.05 (m, 5H), 1.80-2.15 (m, 7H), 1.50 (dd, J=6.80, 25.20 Hz, 3H), 1.26 (dd, J=6.80, 25.60 Hz, 3H).

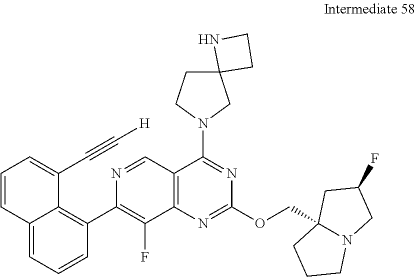 [SMILES: C#Cc1cccc2cccc(-c3ncc4c(N5CCC6(CCN6)C5)nc(OC[C@@]56CCCN5C[C@H](F)C6)nc4c3F)c12]

### 7-(8-ethynylnaphthalen-1-yl)-8-fluoro-2-(((2R, 7aS)-2-fluorotetrahydro-1H-pyrrolizin-7a(5H)-yl)methoxy)-4-(1,6-diazaspiro[3.4]octan-6-yl)pyrido[4,3-d]pyrimidine

### Isomer 1 and Isomer 2

These intermediates were prepared following the general procedure above using tert-butyl 1,6-diazaspiro[3.4]octane-1-carboxylate in place of tert-butyl (2S,5R)-2,5-dimethylpiperazine-1-carboxylate. Additionally, the initial material provided by these steps was further purified by preparative SFC (column: Chiralcel AD-H, 4.6 mm×250 mm, 5 m particles; flow rate: 100 mL/min; column temperature: 40° C.; isocratic: 55% C₂:45% 1:1 MeCN/MeOH with 0.2% ammonia.

Isomer 1: LC/MS (ESI) m/z: [M+H]⁺ calcd for C₃₃H₃₃F₂N₆O 567.3; found 567.0; ¹H NMR (400 MHz, DMSO-d₆): δ 9.25-9.20 (m, 1H), 8.16-8.12 (m, 2H), 7.73-7.68 (m, 2H), 7.60-7.54 (m, 2H), 5.28 (d, J=52.40 Hz, 1H), 4.93 (s, 1H), 4.14-4.01 (m, 4H), 3.88-3.76 (m, 1H), 3.02-2.80 (m, 5H), 2.25-2.01 (m, 13H).

Isomer 2: LC/MS (ESI) m/z: [M+H]⁺ calcd for C₃₃H₃₃F₂N₆O 567.3; found 567.2; ¹H NMR (400 MHz, DMSO-d₆): δ 9.25-9.20 (m, 1H), 8.14 (t, J=8.00 Hz, 2H), 7.71-7.67 (m, 2H), 7.60-7.56 (m, 2H), 5.28 (d, J=53.60 Hz, 1H), 4.80 (s, 1H), 4.13-4.01 (m, 4H), 3.81-3.64 (m, 1H), 3.11-2.80 (m, 5H), 2.05-1.77 (m, 13H).

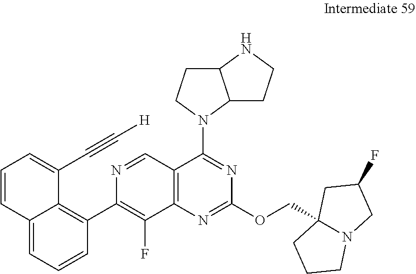 [SMILES: C#Cc1cccc2cccc(-c3ncc4c(N5CCC6NCCC65)nc(OC[C@@]56CCCN5C[C@H](F)C6)nc4c3F)c12]

### 7-(8-ethynylnaphthalen-1-yl)-8-fluoro-2-(((2R, 7aS)-2-fluorotetrahydro-1H-pyrrolizin-7a(5H)-yl)methoxy)-4-(hexahydropyrrolo[3,2-b]pyrrol-1(2H)-yl)pyrido[4,3-d]pyrimidine Isomer 1 and Isomer 2

These intermediates were prepared following the general procedure above using tert-butyl hexahydropyrrolo[3,2-b]pyrrole-1(2H)-carboxylate in place of tert-butyl (2S,5R)-2,5-dimethylpiperazine-1-carboxylate. Additionally, the initial material provided by these steps was further purified by preparative SFC (column: Chiralcel AD-H, 4.6 mm×250 mm, 5 m particles; flow rate: 100 mL/min; column temperature: 40° C.; isocratic: 55% CO₂:45% 1:1 MeCN/MeOH with 0.2% ammonia) to provide two isomers.

Isomer 1: LC/MS (ESI) m/z: [M+H]⁺ calcd for C₃₃H₃₃F₂N₆O 567.3; found 567.0.

Isomer 2: LC/MS (ESI) m/z: [M+H]⁺ calcd for C₃₃H₃₃F₂N₆O 567.3; found 567.2.

General Scheme for the preparation of the following intermediates:

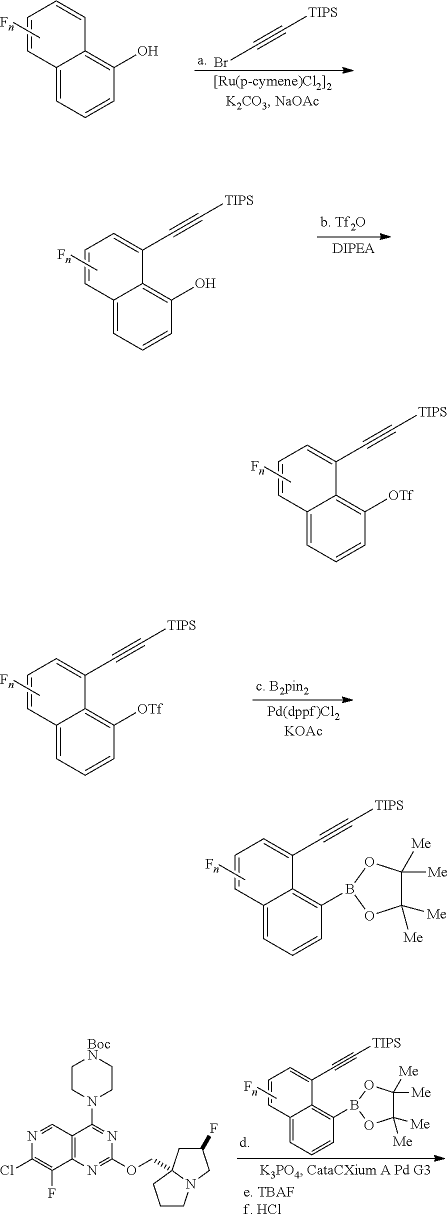

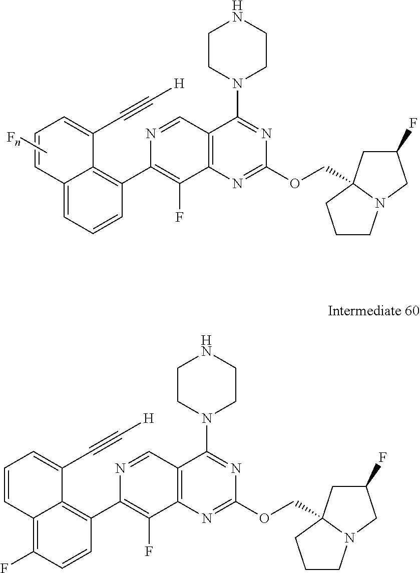

### 7-(8-ethynyl-4-fluoronaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine

### Step 1: Preparation of 4-fluoro-8-((triisopropylsilyl)ethynyl)naphthalen-1-ol

4-Fluoronaphthalen-1-ol (1 g, 6.17 mmol) and (bromomethyl)triisopropylsilane (1.93 g, 7.40 mmol) were dissolved in DCE (15 mL). Potassium carbonate (0.85 g, 6.17 mmol), sodium acetate (0.10 g, 1.23 mmol), and dichloro(p-cymene)ruthenium(II) dimer (0.94 g, 1.542 mmol) were added, and the reaction mixture was heated at 40° C. for 16 h. The reaction mixture was filtered through a pad of diatomaceous earth (Celite®), eluting with additional DCM (2×50 mL). The combined filtrate was concentrated under reduced pressure. The crude residue was purified by column chromatography (0→10% EtOAc/hexanes) to provide the desired product (1.5 g, 4.38 mmol, 71% yield).; ¹H NMR (400 MHz, DMSO-d₆): δ 10.03 (s, 1H), 7.96 (t, J=7.60 Hz, 1H), 7.69 (dd, J=1.20, 7.20 Hz, 1H), 7.52 (dd, J=7.20, 8.40 Hz, 1H), 7.18 (dd, J=8.40, 10.40 Hz, 1H), 6.83 (dd, J=4.80, 8.40 Hz, 1H), 1.14 (s, 21H).

### Step 2: Preparation of 4-fluoro-8-((triisopropylsilyl)ethynyl) naphthalen-1-yl trifluoromethanesulfonate

4-Fluoro-8-((triisopropylsilyl)ethynyl)naphthalen-1-ol (500 mg, 1.460 mmol) was dissolved in DCM (10 mL), and DIPEA (0.765 mL, 4.38 mmol) was added. The solution was cooled to −40° C., and triflic anhydride (0.370 mL, 2.190 mmol) was added dropwise at this temperature. The reaction mixture was allowed to warm to −20° C. and was stirred for 1 h. The reaction mixture was quenched with cold water (20 mL) DCM (30 mL) was added. The layers were separated, and the aqueous phase was further extracted with DCM (2×30 mL). The combined organic layers were dried over sodium sulfate, filtered, and concentrated. The crude residue was purified by column chromatography (5% EtOAc/hexanes) to provide the desired product (500 mg, 1.01 mmol, 69% yield) as a colorless gel.; ¹H NMR (400 MHz, DMSO-d₆): δ 8.22 (d, J=8.80 Hz, 1H), 8.02 (dd, J=1.20, 7.40 Hz, 1H), 7.78-7.74 (m, 2H), 7.58 (t, J=8.80 Hz, 1H), 1.20-1.12 (m, 21H).

### Step 3: Preparation of ((5-fluoro-8-(4,4,5,5-tetramethyl-1,3,2-dioxaborolan-2-yl)naphthalen-1-yl)ethynyl)triisopropylsilane

4-Fluoro-8-((triisopropylsilyl)ethynyl)naphthalen-1-yl trifluoromethanesulfonate (500 mg, 1.054 mmol) was dissolved in dioxane (10 mL) and B₂pin₂ (400 mg, 1.56 mmol) and potassium acetate (310 mg, 3.16 mmol) were added. Nitrogen was sparged through the solution for 5 min, and Pd(dppf)Cl₂ (77 mg, 0.10 mmol) was added. The reaction mixture was heated at 100° C. for 24 h. Cold water (50 mL) and EtOAc (50 mL) were added, and the layers were separated. The aqueous phase was further extracted with EtOAc (2×50 mL). The combined organic layers were dried over sodium sulfate, filtered, and concentrated. The crude residue was purified by column chromatography (extracted with ethyl acetate (3×50 mL). The organic layer was dried over anhydrous sodium sulphate, filtered, and the filtrate was concentrated under reduced pressure to get the crude product. The crude residue was purified by column chromatography (5→10% EtOAc/hexanes) to provide the desired product (400 mg, 0.813 mmol, 77% yield).

### Step 4: Preparation of tert-butyl 4-(8-fluoro-7-(4-fluoro-8-((triisopropylsilyl)ethynyl)naphthalen-1-yl)-2-(((2R, 7aS)-2-fluorotetrahydro-1H-pyrrolizin-7a(5H)-yl)methoxy)pyrido[4,3-d]pyrimidin-4-yl)piperazine-1-carboxylate

tert-Butyl 4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (250 mg, 0.476 mmol) and ((5-fluoro-8-(4,4,5,5-tetramethyl-1,3,2-dioxaborolan-2-yl)naphthalen-1-yl)ethynyl)triisopropylsilane (539 mg, 0.714 mmol) were combined and dissolved in in dioxane (5 mL). Aqueous potassium phosphate solution (2.0 M, 0.95 mL, 1.905 mmol) was added, and nitrogen was sparged through the solution for 5 min. CataCXium A Pd G3 (35 mg, 0.048 mmol) was added, and the reaction mixture was heated at 100° C. for 1 h. Cold water (10 mL) and EtOAc (25 mL) were added, and the layers were separated. The aqueous phase was further extracted with EtOAc (25 mL). The combined organic layers were dried over sodium sulfate, filtered, and concentrated. The crude residue was purified by column chromatography to provide the desired product (400 mg, 0.314 mmol, 66% yield) as off white solid. LCMS (ESI) m/z: [M+H]⁺ calcd for C₄₅H₅₈F₃N₆O₃Si 815.4; found 815.2.

### Step 5: Preparation of tert-butyl 4-(7-(8-ethynyl-4-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate

tert-Butyl 4-(8-fluoro-7-(4-fluoro-8-((triisopropylsilyl)ethynyl)naphthalen-1-yl)-2-(((2R, 7aS)-2-fluorotetrahydro-1H-pyrrolizin-7a(5H)-yl)methoxy)pyrido[4,3-d]pyrimidin-4-yl)piperazine-1-carboxylate (350 mg, 0.275 mmol) was dissolved in THE (10 mL), and the solution was cooled to 0° C. TBAF solution (1.0 M in THF, 0.28 mL, 0.28 mmol) was added, and the reaction mixture was stirred for 1 h. Cold water (20 mL) and EtOAc 50 mL) were added, and the layers were separated. The organic layer was dried over sodium sulfate, filtered, and concentrated. The crude residue was purified by column chromatography on neutral alumina (35% EtOAc/hexanes) to provide the desired product (200 mg, 0.18 mmol, 66% yield) as an off white solid. LCMS (ESI) m/z: [M+H]⁺ calcd for C₃₆H₃₈F₃N₆O₃ 659.3; found 659.2.

### Step 6: Preparation of 7-(8-ethynyl-4-fluoronaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine

tert-Butyl 4-(7-(8-ethynyl-4-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (200 mg, 0.304 mmol) was dissolved in MeCN (5 mL), and the solution was cooled to 0° C. HCl solution (4.0 M in dioxane, 0.076 mL, 0.30 mmol) was added dropwise, and the reaction mixture was stirred for 2 h. The reaction mixture was concentrated, and the crude residue was purified by prep HPLC was added 4N HCl in 1,4-Dioxane (0.076 mL, 0.304 mmol) at 0° C. The reaction mixture was stirred for 2 h at this temperature. After completion, the reaction mixture was concentrated under reduced pressure. The residue was purified by prep-HPLC [Diluent :THF:ACN:WATER (10:60:30); Column: X-Select C₁₈ (150 x19)mm, 5 micron; Temperature: Ambient; Mobile phase A: 5 mM Ammonium formate; Mobile phase B: acetonitrile; Flow :15 mL/min] to provide the desired product as its formic acid salt. This material was partitioned between EtOAc (20 mL) and saturated aqueous NaHCO₃solution (10 mL). The layers were separated, and the organic phase was dried over sodium sulfate, filtered, and concentrated. The residue was lyophilized to provide the desired product (30 mg, 0.054 mmol, 16% yield) as an off white solid. LCMS (ESI) m/z: [M+H]⁺ calcd for C₃₁H₃₀F₃N₆O 559.2; found 559.1; ¹H NMR (400 MHz, DMSO-d₆): 1H-NMR (400 MHz, DMSO-d₆): δ 9.04 (s, 1H), 8.26 (d, J=8.40 Hz, 1H), 7.83 (s, 1H), 7.69 (t, J=7.60 Hz, 1H), 7.63 (t, J=6.00 Hz, 1H), 7.57 (t, J=8.00 Hz, 1H), 5.28 (d, J=54.40 Hz, 1H), 4.01-4.15 (m, 2H), 3.88-3.93 (m, 4H), 3.82 (s, 1H), 2.84-3.08 (m, 9H), 2.01-2.14 (m, 3H), 1.77-1.83 (m, 3H).

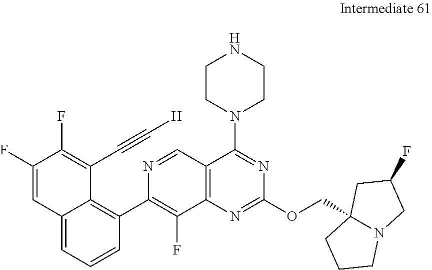 [SMILES: C#Cc1c(F)c(F)cc2cccc(-c3ncc4c(N5CCNCC5)nc(OC[C@@]56CCCN5C[C@H](F)C6)nc4c3F)c12]

### 7-(8-ethynyl-6,7-difluoronaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine

This intermediate was prepared following the general procedure above using 6,7-difluoronaphthalen-1-ol in place of 4-fluoronaphthalen-1-ol. LCMS (ESI) m/z: [M+H]* calcd for C₃₁H₂₉F₄N₆O 577.2; found 577.2; ¹H NMR (400 MHz, DMSO-d₆): δ 9.05 (s, 1H), 8.28-8.16 (m, 4H), 7.75 (t, J=8.00 Hz, 1H), 7.66 (d, J=7.20 Hz, 1H), 5.28 (d, J=54.00 Hz, 1H), 4.27 (s, 1H), 4.16-4.04 (m, 2H), 3.93-3.88 (m, 4H), 3.10-2.84 (m, 8H), 2.15-1.78 (m, 5H).

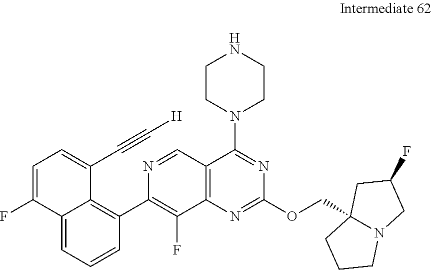 [SMILES: C#Cc1ccc(F)c2cccc(-c3ncc4c(N5CCNCC5)nc(OC[C@@]56CCCN5C[C@H](F)C6)nc4c3F)c12]

### 7-(8-ethynyl-5-fluoronaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine

This intermediate was prepared following the general procedure above using 5-fluoronaphthalen-1-ol in place of 4-fluoronaphthalen-1-ol. LCMS (ESI) m/z: [M+H]⁺ calcd for C₃₁H₃₀F₃N₆O 559.2; found 559.2; ¹H NMR (400 MHz, DMSO-d₆): δ 9.05 (s, 1H), 8.30 (d, J=8.40 Hz, 1H), 8.17 (d, J=6.80 Hz, 1H), 7.84 (t, J=0.80 Hz, 1H), 7.74-7.72 (m, 2H), 7.45 (q, J=8.00 Hz, 1H), 5.22 (d, J=52 Hz, 1H), 4.15-4.06 (m, 2H), 3.93-3.88 (m, 5H), 3.11-2.99 (m, 8H), 2.84 (d, J=6.40 Hz, 1H), 2.14-2.01 (m, 2H), 1.83-0.78 (m, 3H).

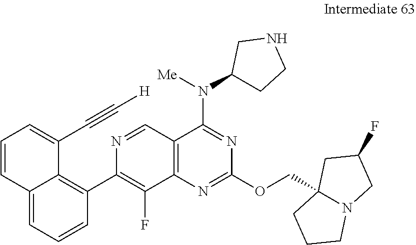 [SMILES: C#Cc1cccc2cccc(-c3ncc4c(N(C)[C@@H]5CCNC5)nc(OC[C@@]56CCCN5C[C@H](F)C6)nc4c3F)c12]

### 8-(8-fluoro-2-(((2R, 7aS)-2-fluorotetrahydro-1H-pyrrolizin-7a(5H)-yl)methoxy)-4-(methyl((R)-pyrrolidin-3-yl)amino)pyrido[4,3-d]pyrimidin-7-yl)-1-naphthonitrile

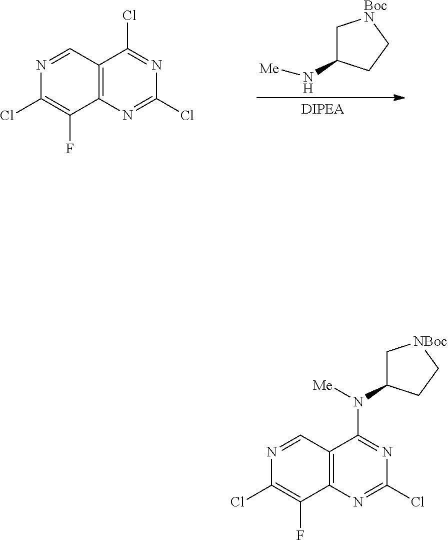 [SMILES: Fc1c(Cl)ncc2c(Cl)nc(Cl)nc12 ; CN[C@@H]1CCN(C(=O)OC(C)(C)C)C1 ; CN(c1nc(Cl)nc2c(F)c(Cl)ncc12)[C@@H]1CCN(C(=O)OC(C)(C)C)C1]

### Step 1: Preparation of tert-butyl (R)-3-((2,7-dichloro-8-fluoropyrido[4,3-d]pyrimidin-4-yl)(methyl)amino)pyrrolidine-1-carboxylate

A stirred solution of 2,4,7-trichloro-8-fluoropyrido[4,3-d]pyrimidine (320 mg, 1.268 mmol) in DCM (10 mL) was cooled at 0° C. and to it was added DIPEA (0.487 mL, 2.79 mmol) followed by a solution of tert-butyl (R)-3-(methylamino)pyrrolidine-1-carboxylate (254 mg, 1.268 mmol) in DCM (2 mL). Stirring was continued at 0° C. for 1 h. The mixture was warmed to room temperature and concentrated. The resulting residue was purified by flash column chromatography (gradient 5→100% EtOAc/hexanes) to provide the desired product (453 mg, 1.088 mmol, 86% yield). LC/MS (ESI) m/z: [M+H]* calcd for C₁₇H₂₀Cl₂FN₅O₂ 416.1; found 416.1; ¹H NMR (500 MHz, CDCl₃) δ 9.02 (s, 1H), 5.45 (br t, J=7.8 Hz, 1H), 3.86 (br t, J=9.6 Hz, 1H), 3.76-3.64 (m, 1H), 3.48-3.40 (m, 2H), 3.46 (s, 3H), 2.42-2.32 (m, 1H), 2.24-2.15 (m, 1H), 1.51 (s, 9H).

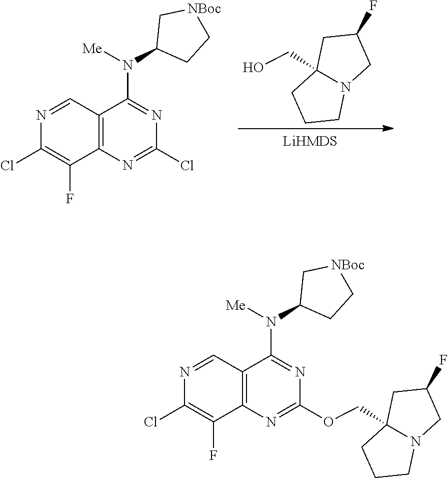 [SMILES: CN(c1nc(Cl)nc2c(F)c(Cl)ncc12)[C@@H]1CCN(C(=O)OC(C)(C)C)C1 ; OC[C@@]12CCCN1C[C@H](F)C2 ; CN(c1nc(OC[C@@]23CCCN2C[C@H](F)C3)nc2c(F)c(Cl)ncc12)[C@@H]1CCN(C(=O)OC(C)(C)C)C1]

### Step 2: tert-butyl (R)-3-((2,7-dichloro-8-fluoropyrido[4,3-d]pyrimidin-4-yl)(methyl)amino)pyrrolidine-1-carboxylate

To a stirred solution of tert-butyl (R)-3-((2,7-dichloro-8-fluoropyrido[4,3-d]pyrimidin-4-yl)(methyl)amino)pyrrolidine-1-carboxylate (300 mg, 0.72 mmol) and ((2R, 7aS)-2-fluorotetrahydro-1H-pyrrolizin-7a(5H)-yl)methanol, HCl (155 mg, 0.79 mmol) in anhydrous THE (5 mL) was added LiHMDS solution (1.0 M in THF, 1.80 mL, 1.80 mmol) dropwise via syringe. The mixture was stirred for 18 h, was partially concentrated and was purified by flash column chromatography (gradient 50→100% EtOAc with 5% Et₃N/hexanes) to provide the desired product (256 mg, 0.475 mmol, 66% yield). LC/MS (ESI) m/z: [M+H]⁺ calcd for C₂₅H₃₃C₁F₂N₆O₃ 539.2; found 539.3; ¹H NMR (500 MHz, CDCl₃) δ 8.88 (s, 1H), 5.41-5.20 (m, 2H), 4.35-4.27 (m, 1H), 4.25 (s, 1H), 3.84 (dd, J=11.5, 8.1 Hz, 1H), 3.74-3.56 (m, 1H), 3.51-3.40 (m, 2H), 3.38 (s, 3H), 3.34-3.16 (m, 3H), 3.00 (br d, J=9.2 Hz, 1H), 2.38-2.08 (m, 5H), 2.02-1.84 (m, 3H), 1.51 (s, 9H).

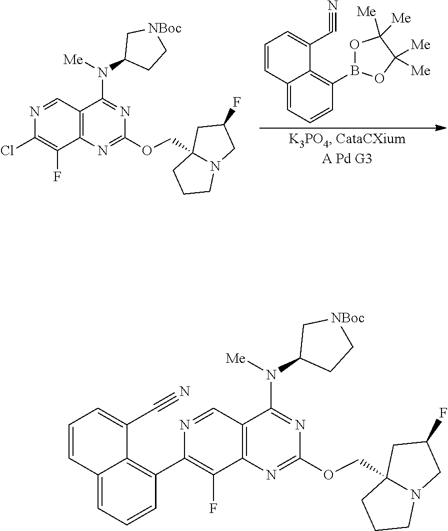 [SMILES: CN(c1nc(OC[C@@]23CCCN2C[C@H](F)C3)nc2c(F)c(Cl)ncc12)[C@@H]1CCN(C(=O)OC(C)(C)C)C1 ; CC1(C)OB(c2cccc3cccc(C#N)c23)OC1(C)C ; "K3PO4, CataCXiumA Pd G3" ; CN(c1nc(OC[C@@]23CCCN2C[C@H](F)C3)nc2c(F)c(-c3cccc4cccc(C#N)c34)ncc12)[C@@H]1CCN(C(=O)OC(C)(C)C)C1]

### Step 3: Preparation of tert-butyl (R)-3-((7-(8-cyanonaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

A mixture of tert-butyl (R)-3-((7-chloro-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (30 mg, 0.056 mmol), 8-(4,4,5,5-tetramethyl-1,3,2-dioxaborolan-2-yl)-1-naphthonitrile (23.30 mg, 0.083 mmol) and a solution of potassium phosphate tribasic (1.5 M in water, 0.11 mL, 0.167 mmol) were stirred in degassed Dioxane (1.5 mL) in a pressure vial. Methanesulfonato(diadamantyl-n-butylphosphino)-2′-amino-1,1′-biphenyl-2-yl)palladium(II) dichloromethane adduct (cataCXium A Palladacycle Gen3) (4.05 mg, 5.57 μmol) was added and the vial was sealed. The atmosphere was evacuated and replaced with nitrogen (3 times). The mixture was heated at 80° C. for 3 hours, concentrated and directly purified by flash column chromatography (50->100% EtOAc w/5% Et₃N/hexanes) to provide the desired product (21 mg, 0.032 mmol, 58% yield). LC/MS (ESI) m/z: [M+H]⁺ calcd for C₃₆H₃₉F₂N₇O₃ 656.3; found 656.3.

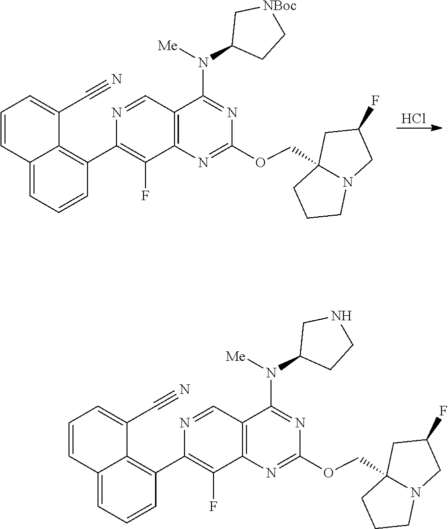 [SMILES: CN(c1nc(OC[C@@]23CCCN2C[C@H](F)C3)nc2c(F)c(-c3cccc4cccc(C#N)c34)ncc12)[C@@H]1CCN(C(=O)OC(C)(C)C)C1 ; Cl ; CN(c1nc(OC[C@@]23CCCN2C[C@H](F)C3)nc2c(F)c(-c3cccc4cccc(C#N)c34)ncc12)[C@@H]1CCNC1]

### Step 4: Preparation of 8-(8-fluoro-2-(((2R, 7aS)-2-fluorotetrahydro-1H-pyrrolizin-7a(5H)-yl)methoxy)-4-(methyl((R)-pyrrolidin-3-yl)amino)pyrido[4,3-d]pyrimidin-7yl)-1-naphthonitrile

To a solution of tert-butyl (R)-3-((7-(8-cyanonaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (21 mg, 0.032 mmol) in acetonitrile (1 mL) was added a solution of HCl (4M in dioxane 0.080 mL, 0.320 mmol). The mixture was stirred for 1 h and then made basic by addition of 1-methylimidazole (0.038 mL, 0.480 mmol). The solution was concentrated and used directly in the next step, without further purification (quantitative yield assumed). LC/MS (ESI) m/z: [M+H]⁺ calcd for C₃₁H₃₁F₂N₇O 556.2; found 556.2.

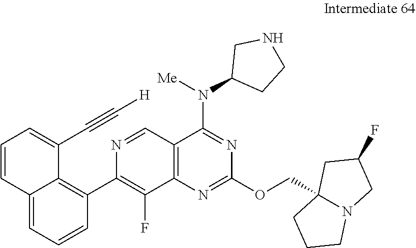 [SMILES: C#Cc1cccc2cccc(-c3ncc4c(N(C)[C@@H]5CCNC5)nc(OC[C@@]56CCCN5C[C@H](F)C6)nc4c3F)c12]

### 7-(8-ethynylnaphthalen-1-yl)-8-fluoro-2-(((2R, 7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine

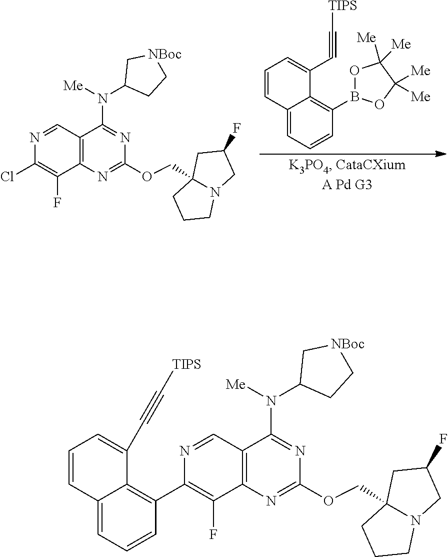

### Step 1: Preparation of tert-butyl (R)-3-((8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)(methyl)amino)pyrrolidine-1-carboxylate

A mixture of tert-butyl (R)-3-((7-chloro-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (50 mg, 0.093 mmol), triisopropyl((8-(4,4,5,5-tetramethyl-1,3,2-dioxaborolan-2-yl)naphthalen-1-yl)ethynyl)silane (60.5 mg, 0.139 mmol) and a solution of potassium phosphate tribasic (1.5 M in water, 0.19 mL, 0.278 mmol) were stirred in degassed Dioxane (1.5 mL) in a pressure vial. Methanesulfonato(diadamantyl-n-butylphosphino)-2′-amino-1,1′-biphenyl-2-yl)palladium(II) dichloromethane adduct (cataCXium A Palladacycle Gen3) (4.8 mg, 9.28 μmol) was added and the vial was sealed. The atmosphere was evacuated and replaced with nitrogen (3 times). The mixture was heated at 80° C. for 3 hours, concentrated and directly purified by column chromatography (50->100% EtOAc w/5% Et₃N/hexanes) to provide the desired product (62 mg, 0.076 mmol, 82.0% yield). LC/MS (ESI) m/z: [M+H]⁺ calcd for C₄₆H₆₀F₂N₆O₃Si 811.4; found 811.4; ¹H NMR (500 MHz, CDCl₃) δ 9.22 (s, 1H), 8.01-7.97 (m, 1H), 7.95 (d, J=8.2 Hz, 1H), 7.83 (d, J=7.3 Hz, 1H), 7.61-7.57 (m, 2H), 7.48 (t, J=7.7 Hz, 1H), 5.32 (s, 3H), 4.65-4.40 (m, 1H), 3.97-3.86 (m, 1H), 3.84-3.51 (m, 3H), 3.38-3.02 (m, 2H), 2.69-2.36 (m, 3H), 2.30-2.05 (m, 3H), 1.80-1.55 (m, 5H), 1.51 (s, 9H), 1.19-1.11 (m, 2H), 0.93-0.83 (m, 18H), 0.58 (quin, J=7.4 Hz, 3H).

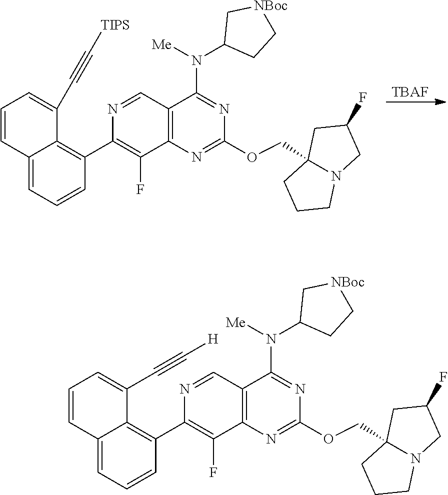

### Step 2: Preparation of tert-butyl (R)-3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

To a stirred solution of tert-butyl (R)-3-((8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)(methyl)amino)pyrrolidine-1-carboxylate (285 mg, 0.351 mmol) in THE (3 mL) was added TBAF (1M in THF, 0.422 mL, 0.422 mmol). The mixture was stirred for 30 min. The mixture was concentrated and was directly purified by column chromatography (50->100% EtOAc w/5% Et₃N/hexanes) to provide the desired product (199 mg, 0.304 mmol, 86% yield). LC/MS (ESI) m/z: [M+H]⁺ calcd for C₃₇H₄₀F₂N₆O_{3 655.3}; found 655.4; ¹H NMR (500 MHz, CDCl₃) δ 9.14 (s, 1H), 8.05-7.92 (m, 2H), 7.77 (d, J=7.1 Hz, 1H), 7.65-7.56 (m, 2H), 7.47 (t, J=7.7 Hz, 1H), 5.44-5.15 (m, 2H), 4.45-4.23 (m, 2H), 3.93-3.83 (m, 1H), 3.76-3.53 (m, 1H), 3.43 (d, J=3.7 Hz, 4H), 3.36-3.15 (m, 3H), 3.07-2.93 (m, 1H), 2.60 (br d, J=7.7 Hz, 1H), 2.42-2.10 (m, 6H), 2.02-1.88 (m, 3H), 1.51 (s, 9H).

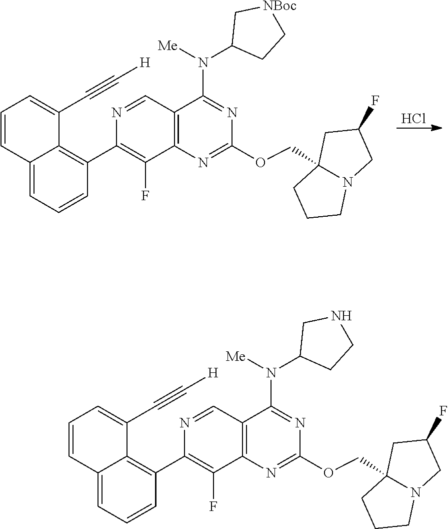 [SMILES: C#Cc1cccc2cccc(-c3ncc4c(N(C)C5CCN(C(=O)OC(C)(C)C)C5)nc(OC[C@@]56CCCN5C[C@H](F)C6)nc4c3F)c12 ; Cl ; C#Cc1cccc2cccc(-c3ncc4c(N(C)C5CCNC5)nc(OC[C@@]56CCCN5C[C@H](F)C6)nc4c3F)c12]

### Step 3: Preparation of 7-(8-ethynylnaphthalen-1-yl)-8-fluoro-2-(((2R, 7aS)-2-fluorotetrahydro-H-pyrrolizin-7a(5H)-yl)methoxy)-N-methyl-N—((R)-pyrrolidin-3-yl)pyrido[4,3-d]pyrimidin-4-amine

To a solution of tert-butyl (R)-3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (140 mg, 0.214 mmol) in acetonitrile (2 mL) was added a solution of HCl (4M in dioxane 0.54 mL, 2.140 mmol). The mixture was stirred for 1 h and then made basic by addition of 1-methylimidazole (0.26 mL, 3.210 mmol). The solution was concentrated and used directly in the next step, without further purification (quantitative yield assumed). LC/MS (ESI) m/z: [M+H]⁺ calcd for C₃₂H₃₂F₂N₆O 555.3; found 555.4.

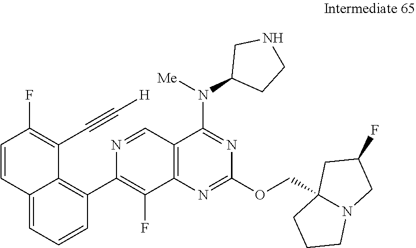 [SMILES: C#Cc1c(F)ccc2cccc(-c3ncc4c(N(C)[C@@H]5CCNC5)nc(OC[C@@]56CCCN5C[C@H](F)C6)nc4c3F)c12]

### 7-(8-ethynyl-7-fluoronaphthalen-1-yl)-8-fluoro-2-(((2R, 7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine

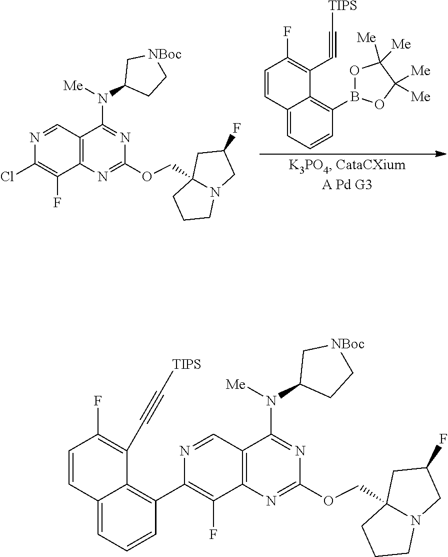

### Step 1: Preparation of tert-butyl (R)-3-((8-fluoro-7-(7-fluoro-8-((triisopropylsilyl)ethynyl)naphthalen-1-yl)-2-(((2R, 7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

A mixture of tert-butyl (R)-3-((7-chloro-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (31.8 mg, 0.059 mmol), ((2-fluoro-8-(4,4,5,5-tetramethyl-1,3,2-dioxaborolan-2-yl)naphthalen-1-yl)ethynyl)triisopropylsilane (40 mg, 0.088 mmol) and a solution of potassium phosphate tribasic (1.5 M in water, 0.12 mL, 0.177 mmol) were stirred in degassed dioxane (1.5 mL) in a pressure vial. Methanesulfonato(diadamantyl-n-butylphosphino)-2′-amino-1,1′-biphenyl-2-yl)palladium(II) dichloromethane adduct (cataCXium A Palladacycle Gen3) (4.3 mg, 5.89 μmol) was added and the vial was sealed. The atmosphere was evacuated and replaced with nitrogen (3 times). The mixture was heated at 80° C. for 3 hours, concentrated and directly purified by column chromatography (50->100% EtOAc w/5% Et₃N/hexanes) to provide the desired product (62 mg, 0.076 mmol, 82.0% yield). LC/MS (ESI) m/z: [M+H]⁺ calcd for C₄₆H₅₉F₃N₆O₃Si 829.7; found 829.4; ¹HNMR (500 MHz, CDCl₃) δ 9.30-9.15 (m, 1H), 7.97 (br d, J=8.0 Hz, 2H), 7.66-7.51 (m, 2H), 7.36 (s, 1H), 5.63-5.36 (m, 3H), 3.95-3.66 (m, 3H), 3.60-3.46 (m, 2H), 3.45 (s, 3H), 3.44-3.04 (m, 4H), 2.72-2.23 (m, 8H), 1.51 (s, 9H), 0.99-0.83 (m, 18H), 0.65-0.49 (m, 3H).

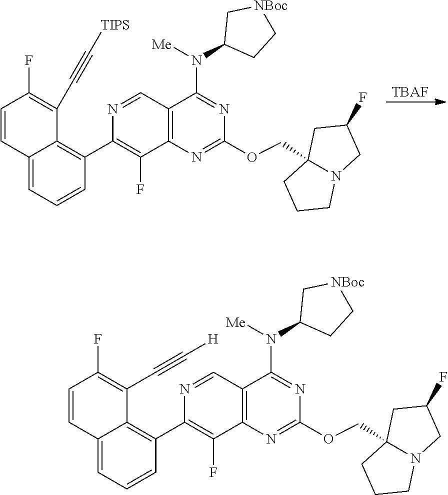

### Step 2: Preparation of tert-butyl (R)-3-((7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

To a stirred solution of tert-butyl (R)-3-((8-fluoro-7-(7-fluoro-8-((triisopropylsilyl)ethynyl)naphthalen-1-yl)-2-(((2R, 7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (50 mg, 0.060 mmol) in THE (1 mL) was added TBAF (1M in THF, 0.072 mL, 0.072 mmol). The mixture was stirred for 30 min. The mixture was concentrated and was directly purified by column chromatography (50->100% EtOAc w/5% Et₃N/hexanes) to provide the desired product (22 mg, 0.033 mmol, 54% yield). LC/MS (ESI) m/z: [M+H]* calcd for C₃₇H₃₉F₃N₆O₃ 673.3; found 673.3

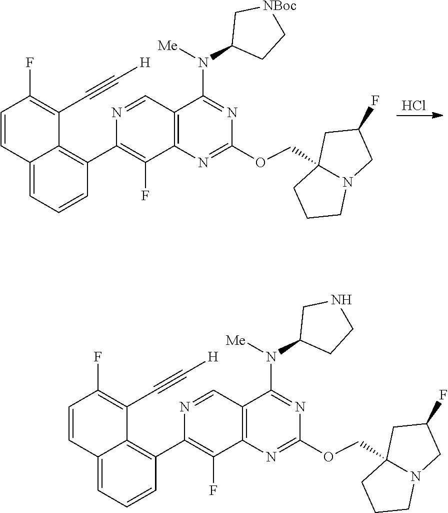 [SMILES: C#Cc1c(F)ccc2cccc(-c3ncc4c(N(C)[C@@H]5CCN(C(=O)OC(C)(C)C)C5)nc(OC[C@@]56CCCN5C[C@H](F)C6)nc4c3F)c12 ; Cl ; C#Cc1c(F)ccc2cccc(-c3ncc4c(N(C)[C@@H]5CCNC5)nc(OC[C@@]56CCCN5C[C@H](F)C6)nc4c3F)c12]

### Step 3: Preparation of 7-(8-ethynyl-7-fluoronaphthalen-1-yl)-8-fluoro-2-(((2R, 7aS)-2-fluorotetrahydro-H-pyrrolizin-7a(5H)-yl)methoxy)-N-methyl-N—((R)-pyrrolidin-3-yl)pyrido[4,3-d]pyrimidin-4-amine

To a solution of tert-butyl (R)-3-((7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (22 mg, 0.033 mmol) in acetonitrile (1 mL) was added a solution of HCl (4M in dioxane, 0.082 mL, 0.327 mmol). The mixture was stirred for 1 h and then made basic by addition of 1-methylimidazole (0.039 mL, 0.491 mmol). The solution was concentrated and used directly in the next step, without further purification (quantitative yield assumed). LC/MS (ESI) m/z: [M+H]⁺ calcd for C₃₂H₃₁F₃N₆O 573.3; found 573.4.

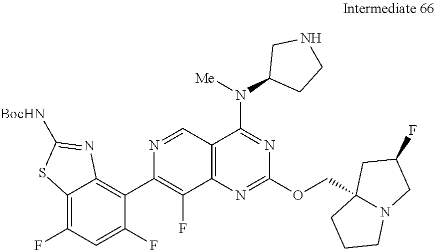 [SMILES: CN(c1nc(OC[C@@]23CCCN2C[C@H](F)C3)nc2c(F)c(-c3c(F)cc(F)c4sc(NC(=O)OC(C)(C)C)nc34)ncc12)[C@@H]1CCNC1]

### tert-butyl (5,7-difluoro-4-(8-fluoro-2-(((7aS)-2-fluorotetrahydro-1H-pyrrolizin-7a(5H)-yl)methoxy)-4-(methyl((R)-pyrrolidin-3-yl)amino)pyrido[4,3-d]pyrimidin-7-yl)benzo[d]thiazol-2-yl)carbamate

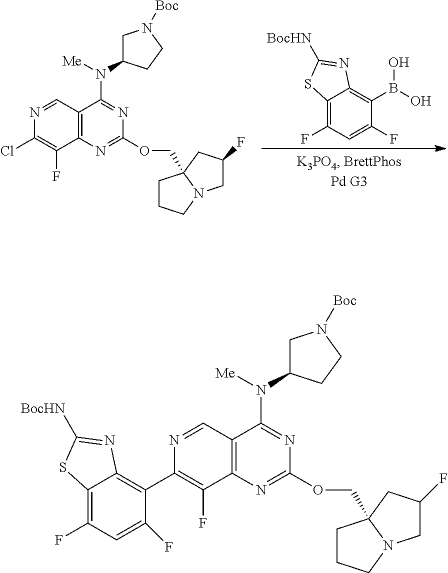 [SMILES: CN(c1nc(OC[C@@]23CCCN2C[C@H](F)C3)nc2c(F)c(Cl)ncc12)[C@@H]1CCN(C(=O)OC(C)(C)C)C1 ; CC(C)(C)OC(=O)Nc1nc2c(B(O)O)c(F)cc(F)c2s1 ; CN(c1nc(OC[C@@]23CCCN2CC(F)C3)nc2c(F)c(-c3c(F)cc(F)c4sc(NC(=O)OC(C)(C)C)nc34)ncc12)[C@@H]1CCN(C(=O)OC(C)(C)C)C1]

### Step 1: Preparation of tert-butyl (3R)-3-((7-(2-((tert-butoxycarbonyl)amino)-5,7-difluorobenzo[d]thiazol-4-yl)-8-fluoro-2-(((7aS)-2-fluorotetrahydro-1H-pyrrolizin-7a(5H)-yl)methoxy)pyrido[4,3-d]pyrimidin-4-yl)(methyl)amino)pyrrolidine-1-carboxylate

A mixture of tert-butyl (R)-3-((7-chloro-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (33 mg, 0.061 mmol), (2-((tert-butoxycarbonyl)amino)-5,7-difluorobenzo[d]thiazol-4-yl)boronic acid (20 mg, 0.061 mmol) and a solution of potassium phosphate tribasic (1.5 M in water, 0.12 mL, 0.182 mmol) were stirred in degassed dioxane (1.0 mL) in a pressure vial. [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate (BRETTPHOS PD G3) (5.5 mg, 6.06 μmol) was added and the vial was sealed. The atmosphere was evacuated and replaced with nitrogen (3 times). The mixture was heated at 85° C. for 3 hours, concentrated and directly purified by column chromatography (50->100% EtOAc w/5% Et₃N/hexanes) to provide the desired product (34 mg, 0.043 mmol, 71% yield). LC/MS (ESI) m/z: [M+H]* calcd for C₃₇H₄₄F₄N₈O₅S 789.3; found 789.6.

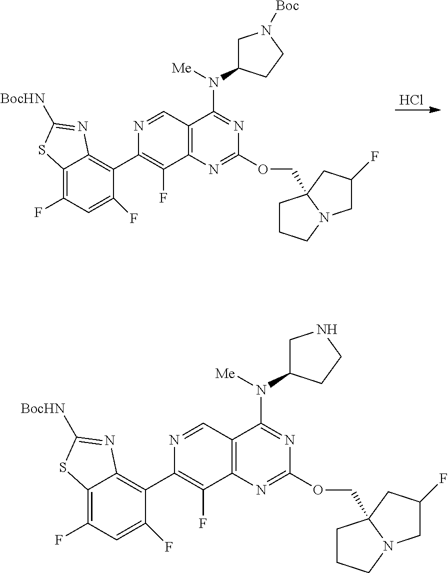 [SMILES: CN(c1nc(OC[C@@]23CCCN2CC(F)C3)nc2c(F)c(-c3c(F)cc(F)c4sc(NC(=O)OC(C)(C)C)nc34)ncc12)[C@@H]1CCN(C(=O)OC(C)(C)C)C1 ; Cl ; CN(c1nc(OC[C@@]23CCCN2CC(F)C3)nc2c(F)c(-c3c(F)cc(F)c4sc(NC(=O)OC(C)(C)C)nc34)ncc12)[C@@H]1CCNC1]

### Step 2: tert-butyl (5,7-difluoro-4-(8-fluoro-2-(((7aS)-2-fluorotetrahydro-1H-pyrrolizin-7a(5H)-yl)methoxy)-4-(methyl((R)-pyrrolidin-3-yl)amino)pyrido[4,3-d]pyrimidin-7-yl)benzo[d]thiazol-2-yl)carbamate

To a solution tert-butyl (3R)-3-((7-(2-((tert-butoxycarbonyl)amino)-5,7-difluorobenzo[d]thiazol-4-yl)-8-fluoro-2-(((7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (34 mg, 0.043 mmol) in acetonitrile (1 mL) was added a solution of HCl (4M in dioxane, 0.108 mL, 0.431 mmol). The mixture was stirred for 1 h and was made basic by addition of 1-methylimidazole (0.052 mL, 0.646 mmol). The solution was concentrated and used directly in the next step, without further purification (quantitative yield assumed). LC/MS (ESI) m/z: [M+H]⁺ calcd for C₃₇H₄₄F₄N₈O₅S 689.3; found 689.4.

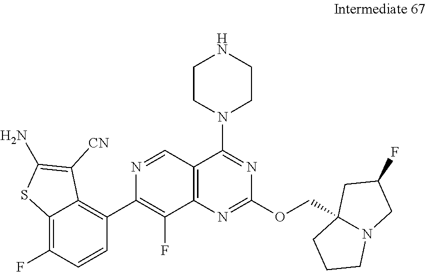 [SMILES: N#Cc1c(N)sc2c(F)ccc(-c3ncc4c(N5CCNCC5)nc(OC[C@@]56CCCN5C[C@H](F)C6)nc4c3F)c12]

### 2-amino-7-fluoro-4-(8-fluoro-2-(((7aS)-2-fluorotetrahydro-1H-pyrrolizin-7a(5H)-yl)methoxy)-4-(piperazin-1-yl)pyrido[4,3-d]pyrimidin-7-yl)benzo[b]thiophene-3-carbonitrile

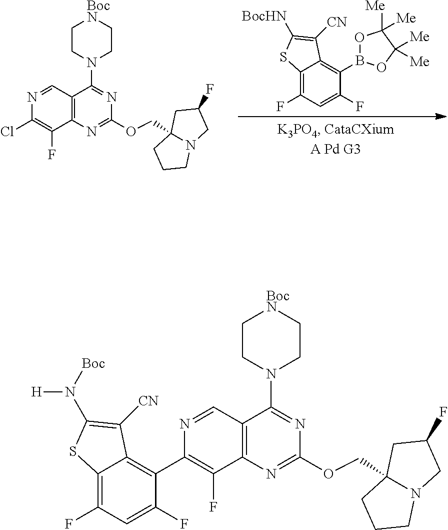 [SMILES: CC(C)(C)OC(=O)N1CCN(c2nc(OC[C@@]34CCCN3C[C@H](F)C4)nc3c(F)c(Cl)ncc23)CC1 ; CC(C)(C)OC(=O)Nc1sc2c(F)cc(F)c(B3OC(C)(C)C(C)(C)O3)c2c1C#N ; CC(C)(C)OC(=O)Nc1sc2c(F)cc(F)c(-c3ncc4c(N5CCN(C(=O)OC(C)(C)C)CC5)nc(OC[C@@]56CCCN5C[C@H](F)C6)nc4c3F)c2c1C#N]

### Step 1: Preparation of tert-butyl 4-(7-(2-((tert-butoxycarbonyl)amino)-3-cyano-7-fluorobenzo[b]thiophen-4-yl)-8-fluoro-2-(((7aS)-2-fluorotetrahydro-1H-pyrrolizin-7a(5H)-yl)methoxy)pyrido[4,3-d]pyrimidin-4-yl)piperazine-1-carboxylate

A mixture of tert-butyl (R)-3-((7-chloro-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidine-1-carboxylate (50 mg, 0.095 mmol), tert-butyl (3-cyano-7-fluoro-4-(4,4,5,5-tetramethyl-1,3,2-dioxaborolan-2-yl)benzo[b]thiophen-2-yl)carbamate (67 mg, 0.162 mmol) and a solution of potassium phosphate tribasic (1.5 M in water, 0.19 mL, 0.286 mmol) were stirred in degassed dioxane (2 mL) in a pressure vial. Methanesulfonato(diadamantyl-n-butylphosphino)-2′-amino-1,1′-biphenyl-2-yl)palladium(II) dichloromethane adduct (cataCXium A Palladacycle Gen3) (6.9 mg, 9.52 μmol) was added and the vial was sealed. The atmosphere was evacuated and replaced with nitrogen (3 times). The mixture was heated at 80° C. for 2 hours, concentrated and purified by reverse phase HPLC (column: Xbridge C18, 19 mm×200 mm, 5 m particles; flow rate: 20 mL/min; column temperature: 25° C.; gradient: 95% 5:95 MeCN:H₂O with 10 mM AA/5% 95:5 MeCN:H₂O with 10 mM AA→100% 95:5 MeCN:H₂O with 10 mM AA; λ=220 nm) to provide the desired product (36.8 mg, 0.470 mmol, 49.5% yield) as a yellow solid. LC/MS (ESI) m/z: [M+H]⁺ calcd for C₃₈H₄₃F₃N₈O₅S 781.3; found 781.6; ¹H NMR (500 MHz, CDCl₃) δ 9.12 (m, 1H), 7.85-7.57 (m, 1H), 7.42 (t, J=8.3 Hz, 0.5H), 7.19 (t, J=8.6 Hz, 0.5H), 5.68-5.39 (m, 1H), 5.12-4.60 (m, 2H), 4.28-3.44 (m, 11H), 3.35-3.10 (m, 1H), 2.93-2.21 (m, 7H), 1.58 (s, 9H), 1.53 (s, 9H).

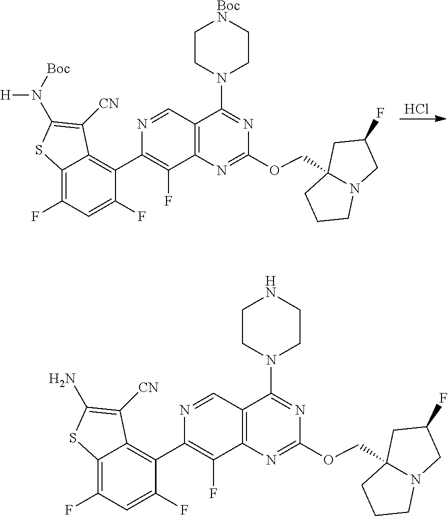 [SMILES: CC(C)(C)OC(=O)Nc1sc2c(F)cc(F)c(-c3ncc4c(N5CCN(C(=O)OC(C)(C)C)CC5)nc(OC[C@@]56CCCN5C[C@H](F)C6)nc4c3F)c2c1C#N ; Cl ; N#Cc1c(N)sc2c(F)cc(F)c(-c3ncc4c(N5CCNCC5)nc(OC[C@@]56CCCN5C[C@H](F)C6)nc4c3F)c12]

### Step 2: Preparation of 2-amino-7-fluoro-4-(8-fluoro-2-(((7aS)-2-fluorotetrahydro-1H-pyrrolizin-7a(5H)-yl)methoxy)-4-(piperazin-1-yl)pyrido[4,3-d]pyrimidin-7-yl)benzo[b]thiophene-3-carbonitrile

To a solution of tert-butyl 4-(7-(2-((tert-butoxycarbonyl)amino)-3-cyano-7-fluorobenzo[b]thiophen-4-yl)-8-fluoro-2-(((7aS)-2-fluorotetrahydro-1H-pyrrolizin-7a(5H)-yl)methoxy)pyrido[4,3-d]pyrimidin-4-yl)piperazine-1-carboxylate (13 mg, 0.017 mmol) in acetonitrile (1 mL) was added a solution of HCl (4M in dioxane, 0.042 mL, 0.166 mmol). The mixture was stirred for 1 h and then made basic by addition of 1-methylimidazole (0.020 mL, 0.250 mmol). The solution was concentrated and used directly in the next step, without further purification (quantitative yield assumed). LC/MS (ESI) m/z: [M+H]⁺ calcd for C₂₈H₂₇F₃N₈OS 581.2; found 581.3.

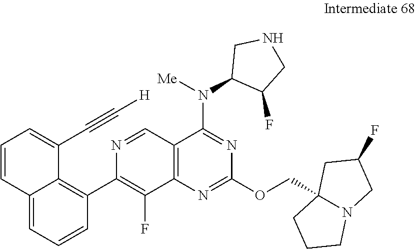 [SMILES: C#Cc1cccc2cccc(-c3ncc4c(N(C)[C@H]5CNC[C@H]5F)nc(OC[C@@]56CCCN5C[C@H](F)C6)nc4c3F)c12]

### 7-(8-ethynylnaphthalen-1-yl)-8-fluoro-N-((3S,4R)-4-fluoropyrrolidin-3-yl)-2-(((2R, 7aS)-2-fluorotetrahydro-1H-pyrrolizin-7a(5H)-yl)methoxy)-N-methylpyrido[4,3-d]pyrimidin-4-amine

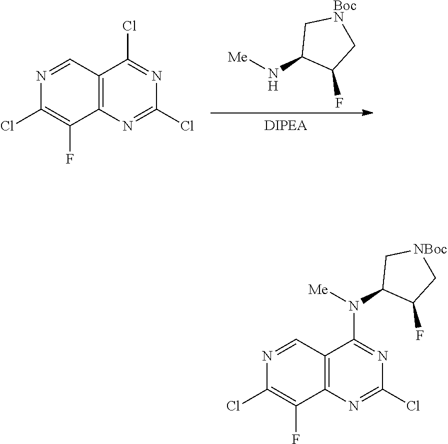

### Step 1: Preparation of tert-butyl (3S,4R)-3-((2,7-dichloro-8-fluoropyrido[4,3-d]pyrimidin-4-yl)(methyl)amino)-4-fluoropyrrolidine-1-carboxylate

A solution of 2,4,7-trichloro-8-fluoropyrido[4,3-d]pyrimidine (375 mg, 1.484 mmol) in DCM (15 mL) was cooled to 0° C. To this solution was added DIPEA (0.570 mL, 3.27 mmol) followed by a solution of tert-butyl (3R, 4S)-3-fluoro-4-(methylamino)pyrrolidine-1-carboxylate (prepared as described in WO2022/173722, 324 mg, 1.484 mmol) in 2 mL CH₂Cl₂. The reaction mixture was stirred at 0° C. for 1 h. The mixture was warmed to room temperature, concentrated, and directly purified by column chromatography (5→100% EtOAc/hexanes) to provide the desired product (415 mg, 0.956 mmol, 64% yield). LC/MS (ESI) m/z: [M+H]⁺ calcd for C₁₇H₁₉C₁₂F₂N₅O₂ 434.1 found 434.1; ¹H NMR (500 MHz, CDCl₃) δ 9.12 (s, 1H), 5.71-5.41 (m, 1H), 5.40-5.15 (m, 1H), 4.01-3.69 (m, 4H), 3.67 (s, 3H), 1.53 (s, 9H).

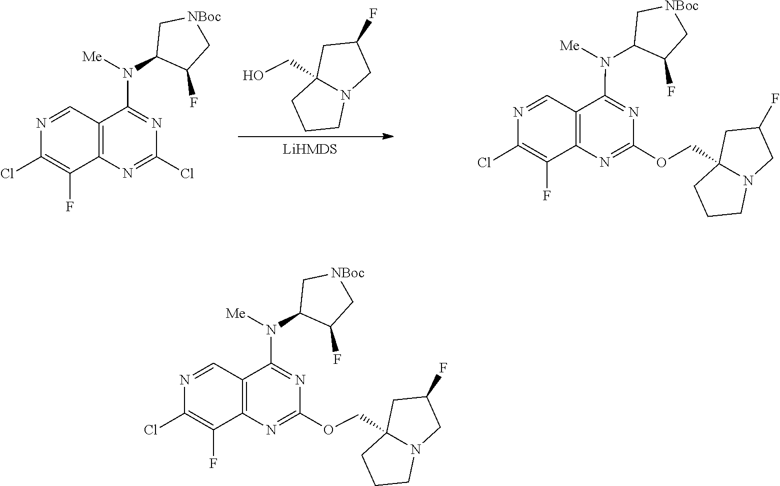 [SMILES: CN(c1nc(Cl)nc2c(F)c(Cl)ncc12)[C@H]1CN(C(=O)OC(C)(C)C)C[C@H]1F ; OC[C@@]12CCCN1C[C@H](F)C2 ; CN(c1nc(OC[C@@]23CCCN2CC(F)C3)nc2c(F)c(Cl)ncc12)C1CN(C(=O)OC(C)(C)C)C[C@H]1F ; CN(c1nc(OC[C@@]23CCCN2C[C@H](F)C3)nc2c(F)c(Cl)ncc12)[C@H]1CN(C(=O)OC(C)(C)C)C[C@H]1F]

### Step 2: Preparation of tert-butyl (3S,4R)-3-((7-chloro-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-4-fluoropyrrolidine-1-carboxylate

To a stirred solution of tert-butyl (3S,4R)-3-((2,7-dichloro-8-fluoropyrido[4,3-d]pyrimidin-4-yl)(methyl)amino)-4-fluoropyrrolidine-1-carboxylate (415 mg, 0.956 mmol) and ((2R, 7aS)-2-fluorotetrahydro-1H-pyrrolizin-7a(5H)-yl)methanol, HCl (206 mg, 1.051 mmol) in anhydrous THE (15 mL) was added LiHMDS (1M in THF, 2.389 mL, 2.389 mmol) dropwise via syringe at room temperature. The mixture was stirred for 18 h, concentrated and directly purified by column chromatography (gradient 50→100% EtOAc with 5% Et₃N/hexanes) to provide the desired product (271 mg, 0.487 mmol, 51% yield). LC/MS (ESI) m/z: [M+H]⁺ calcd for C₂₅H₃₂C₁F₃N₆O₃ 557.2 found 557.4; ¹H NMR (500 MHz, CDCl₃) δ 9.00 (s, 1H), 5.70-5.03 (m, 3H), 4.58-4.30 (m, 1H), 4.14 (q, J=7.1 Hz, 1H), 3.98-3.69 (m, 4H), 3.61 (s, 3H), 3.55-2.83 (m, 4H), 2.56-2.17 (m, 3H), 2.11-1.87 (m, 3H), 1.52 (s, 9H).

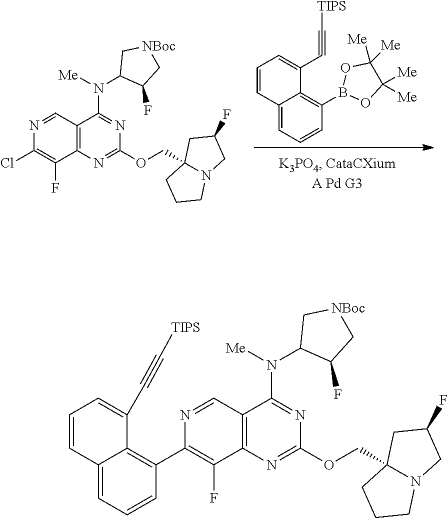

### Step 3: Preparation of tert-butyl (3R, 4S)-3-fluoro-4-((8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)(methyl)amino)pyrrolidine-1-carboxylate

A mixture of tert-butyl (3S,4R)-3-((7-chloro-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-4-fluoropyrrolidine-1-carboxylate (120 mg, 0.215 mmol), triisopropyl((8-(4,4,5,5-tetramethyl-1,3,2-dioxaborolan-2-yl)naphthalen-1-yl)ethynyl)silane (140 mg, 0.323 mmol) and a solution of potassium phosphate tribasic (1.5 M in water, 0.43 mL, 0.646 mmol) were stirred in degassed dioxane (5 mL) in a pressure vial. Methanesulfonato(diadamantyl-n-butylphosphino)-2′-amino-1,1′-biphenyl-2-yl)palladium(II) dichloromethane adduct (cataCXium A Palladacycle Gen3) (15.7 mg, 0.022 mmol) was added and the vial was sealed. The atmosphere was evacuated and replaced with nitrogen (3 times). The mixture was heated at 80° C. for 3 hours, concentrated and directly purified by column chromatography (50->100% EtOAc w/5% Et₃N/hexanes) to provide the desired product (149 mg, 0.180 mmol, 83% yield). LCMS (ESI) m/z: [M+H]⁺ calcd for C₄₆H₅₉F₃N₆O₃Si 829.4 found 829.7; ¹H NMR (500 MHz, CDCl₃) δ 9.42-9.22 (m, 1H), 8.04-7.90 (m, 2H), 7.88-7.78 (m, 1H), 7.66-7.57 (m, 2H), 7.48 (br d, J=3.2 Hz, 1H), 5.85-5.12 (m, 4H), 4.60-4.35 (m, 1H), 4.05-3.75 (m, 4H), 3.67 (br d, J=6.1 Hz, 3H), 3.55-2.96 (m, 4H), 2.15-2.00 (m, 3H), 1.92-1.63 (s, 3H), 1.54 (s, 9H), 0.96-0.81 (m, 18H), 0.70-0.54 (m, 3H).

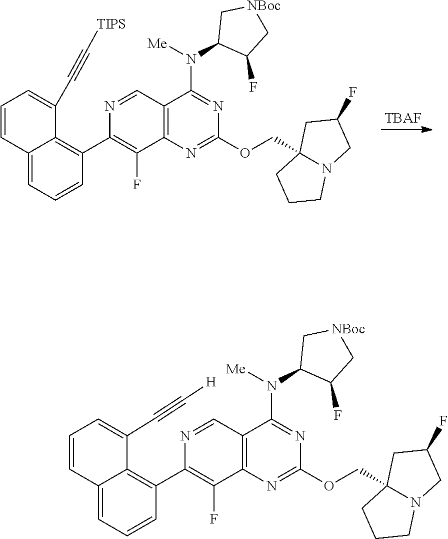

### Step 4: Preparation of tert-butyl (3S,4R)-3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-4-fluoropyrrolidine-1-carboxylate

tert-Butyl (3R, 4S)-3-fluoro-4-((8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)(methyl)amino)pyrrolidine-1-carboxylate (149 mg, 0.180 mmol) was dissolved in THE (2 mL). TBAF solution (1.0 M in THF, 0.216 mL, 0.216 mmol) was added dropwise. After 30 min, the reaction mixture was concentrated, and the crude residue was purified by column chromatography (50→100% EtOAc with 5% Et₃N/hexanes) to provide the desired product (97 mg, 0.144 mmol, 80% yield). LCMS (ESI) m/z: [M+H]⁺ calcd for C₃₇H₃₉F₃N₆O₃ 673.3 found 673.7; ¹H NMR (500 MHz, CDCl₃) δ 9.34-9.22 (m, 1H), 8.06-7.91 (m, 2H), 7.81-7.73 (m, 1H), 7.66-7.55 (m, 2H), 7.51-7.42 (m, 1H), 6.05-5.05 (m, 4H), 4.73-4.47 (m, 1H), 4.18-4.09 (m, 1H), 4.04-3.74 (m, 4H), 3.69 (d, J=6.6 Hz, 3H), 3.55-2.93 (m, 4H), 2.40-2.10 (m, 3H), 1.81-1.59 (m, 3H), 1.54 (s, 9H).

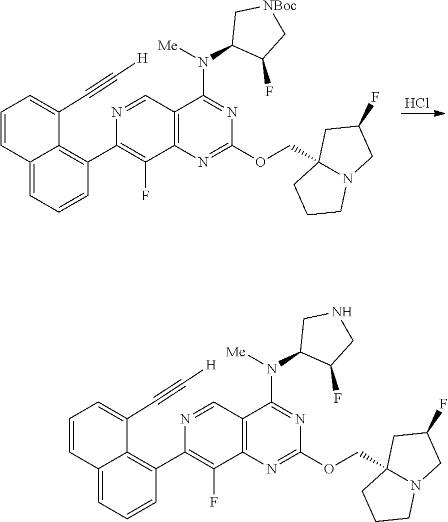 [SMILES: C#Cc1cccc2cccc(-c3ncc4c(N(C)[C@H]5CN(C(=O)OC(C)(C)C)C[C@H]5F)nc(OC[C@@]56CCCN5C[C@H](F)C6)nc4c3F)c12 ; Cl ; C#Cc1cccc2cccc(-c3ncc4c(N(C)[C@H]5CNC[C@H]5F)nc(OC[C@@]56CCCN5C[C@H](F)C6)nc4c3F)c12]

### Step 5: Preparation of 7-(8-ethynylnaphthalen-1-yl)-8-fluoro-N-((3S,4R)-4-fluoropyrrolidin-3-yl)-2-(((2R, 7aS)-2-fluorotetrahydro-1H-pyrrolizin-7a(5H)-yl)methoxy)-N-methylpyrido[4,3-d]pyrimidin-4-amine

To a solution of tert-butyl 3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)azetidine-1-carboxylate (96 mg, 0.143 mmol) in acetonitrile (2 mL) was added a solution of HCl (4M in dioxane, 0.357 mL, 1.427 mmol). The mixture was stirred for 1 h and then made basic by addition of 1-methylimidazole (0.171 mL, 2.140 mmol). The solution was concentrated and used directly in the next step, without further purification (quantitative yield assumed). LC/MS (ESI) m/z: [M+H]⁺ calcd for C₃₂H₃₁F₃N₆O 573.3; found 573.4.

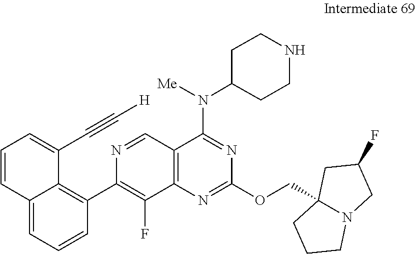 [SMILES: C#Cc1cccc2cccc(-c3ncc4c(N(C)C5CCNCC5)nc(OC[C@@]56CCCN5C[C@H](F)C6)nc4c3F)c12]

### 7-(8-ethynylnaphthalen-1-yl)-8-fluoro-2-(((2R, 7aS)-2-fluorotetrahydro-1H-pyrrolizin-7a(5H)-yl)methoxy)-N-methyl-N-(piperidin-4-yl)pyrido[4,3-d]pyrimidin-4-amine

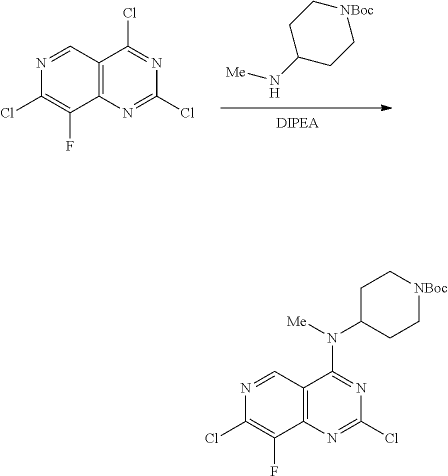

### Step 1: Preparation of tert-butyl 4-((2,7-dichloro-8-fluoropyrido[4,3-d]pyrimidin-4-yl)(methyl)amino)piperidine-1-carboxylate

A solution of 2,4,7-trichloro-8-fluoropyrido[4,3-d]pyrimidine (500 mg, 1.981 mmol) in DCM (15 mL) was cooled to 0° C. To this solution was added DIPEA (0.761 mL, 4.36 mmol) followed by a solution of tert-butyl 4-(methylamino)piperidine-1-carboxylate (424 mg, 1.981 mmol) in 2 mL CH₂C₁₂. The reaction mixture was stirred at 0° C. for 1 h. The mixture was warmed to room temperature, concentrated, and directly purified by column chromatography (5→100% EtOAc/hexanes) to provide the desired product (758 mg, 1.762 mmol, 89% yield). LC/MS (ESI) m/z: [M+H]⁺ calcd for C₁₈H₂₂C₁₂FN₅O₂ 430.1 found 434.1; ¹H NMR (500 MHz, CDCl₃) δ 8.99 (s, 1H), 4.92 (br s, 1H), 4.34 (br d, J=12.5 Hz, 2H), 4.14 (q, J=7.2 Hz, 1H), 3.39 (s, 3H), 2.92 (br t, J=12.3 Hz, 2H), 1.92 (br d, J=11.7 Hz, 2H), 1.82 (qd, J=12.3, 4.4 Hz, 2H), 1.51 (s, 9H).

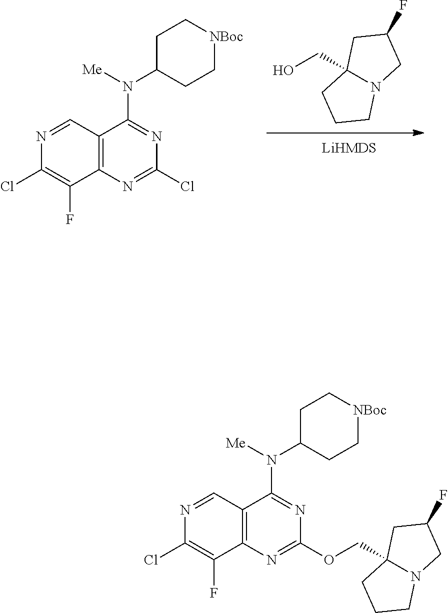

### Step 2: Preparation of tert-butyl 4-((7-chloro-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)piperidine-1-carboxylate

To a stirred solution of tert-butyl 4-((2,7-dichloro-8-fluoropyrido[4,3-d]pyrimidin-4-yl)(methyl)amino)piperidine-1-carboxylate (758 mg, 1.762 mmol) and ((2R, 7aS)-2-fluorotetrahydro-1H-pyrrolizin-7a(5H)-yl)methanol, HCl (379 mg, 1.938 mmol) in anhydrous THE (15 mL) was added LiHMDS (1M in THF, 4.4 mL, 4.40 mmol) dropwise via syringe at room temperature. The mixture was stirred for 18 h, concentrated and directly purified by column chromatography (gradient 50→100% EtOAc with 5% Et₃N/hexanes) to afford the desired product (606 mg, 1.096 mmol, 62% yield). LC/MS (ESI) m/z: [M+H]⁺ calcd for C₂₆H₃₅C₁F₂N₆O₃ 553.2 found 553.5; ¹H NMR (500 MHz, CDCl₃) δ 8.86 (s, 1H), 5.32 (s, 1H), 4.97-4.75 (m, 1H), 4.60-4.21 (m, 4H), 4.14 (q, J=7.1 Hz, 1H), 4.02-3.48 (m, 2H), 3.32 (s, 3H), 2.95 (s, 4H), 2.51-2.15 (m, 3H), 2.16-2.11 (m, 1H), 2.03-1.63 (m, 5H), 1.51 (s, 9H).

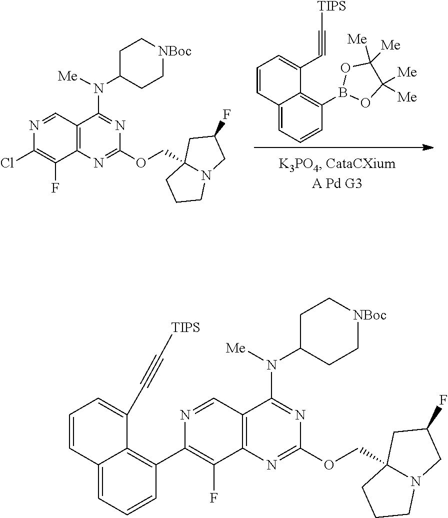

### Step 3: Preparation of tert-butyl 4-((8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)(methyl)amino)piperidine-1-carboxylate

A mixture of tert-butyl 4-((7-chloro-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)piperidine-1-carboxylate (150 mg, 0.271 mmol), triisopropyl((8-(4,4,5,5-tetramethyl-1,3,2-dioxaborolan-2-yl)naphthalen-1-yl)ethynyl)silane (177 mg, 0.407 mmol) and a solution of potassium phosphate tribasic (1.5 M in water, 0.54 mL, 0.814 mmol) were stirred in degassed dioxane (5 mL) in a pressure vial. Methanesulfonato(diadamantyl-n-butylphosphino)-2′-amino-1,1′-biphenyl-2-yl)palladium(II) dichloromethane adduct (cataCXium A Palladacycle Gen3) (19.8 mg, 0.027 mmol) was added and the vial was sealed. The atmosphere was evacuated and replaced with nitrogen (3 times). The mixture was heated at 80° C. for 3 hours, concentrated and directly purified by column chromatography (50->100% EtOAc w/5% Et₃N/hexanes) to provide the desired product (173 mg, 0.210 mmol, 77% yield). LCMS (ESI) m/z: [M+H]⁺ calcd for C₄₇H₆₂F₂N₆O₃Si 825.5 found 825.9; ¹H NMR (500 MHz, CDCl₃) δ 9.17 (d, J=1.4 Hz, 1H), 8.01-7.96 (m, 1H), 7.94 (dd, J=8.3, 1.3 Hz, 1H), 7.82 (d, J=7.1 Hz, 1H), 7.60-7.55 (m, 2H), 7.47 (t, J=7.7 Hz, 1H), 5.48-5.13 (m, 2H), 4.94-4.80 (m, 1H), 4.33 (m, 5H), 3.34 (d, J=3.8 Hz, 3H), 3.22-2.96 (m, 4H), 2.39-2.13 (m, 2H), 2.04-1.78 (m, 8H), 1.51 (s, 9H), 0.92-0.80 (m, 18H), 0.58 (quin, J=7.4 Hz, 3H).

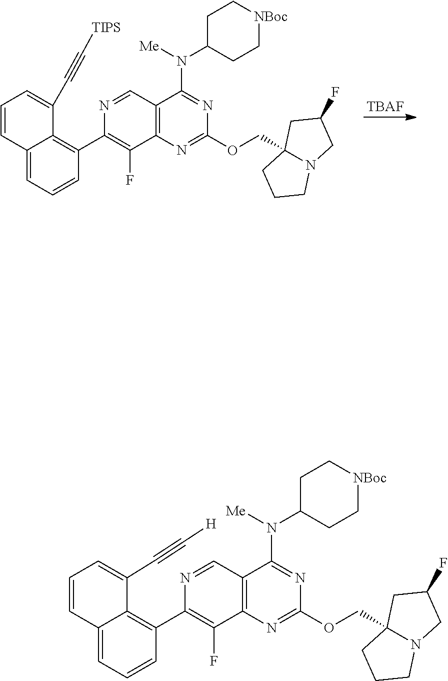

### Step 4: Preparation of tert-butyl 4-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)piperidine-1-carboxylate

tert-Butyl 4-((8-fluoro-2-(((2R, 7aS)-2-fluorotetrahydro-1H-pyrrolizin-7a(5H)-yl)methoxy)-7-(8-((triisopropylsilyl)ethynyl)naphthalen-1-yl)pyrido[4,3-d]pyrimidin-4-yl)(methyl)amino)piperidine-1-carboxylate (173 mg, 0.210 mmol) was dissolved in THE (3 mL). TBAF solution (1.0 M in THF, 0.252 mL, 0.252 mmol) was added dropwise. After 30 min, the reaction mixture was concentrated, and the crude residue was purified by column chromatography (50→100% EtOAc with 5% Et₃N/hexanes) to provide the desired product (93 mg, 0.139 mmol, 66% yield). LCMS (ESI) m/z: [M+H]⁺ calcd for C₃₈H₄₂F₂N₆O₃ 669.3 found 669.6; ¹H NMR (500 MHz, CDCl₃) δ 9.13 (s, 1H), 8.03-7.93 (m, 2H), 7.77 (dd, J=7.2, 1.3 Hz, 1H), 7.67-7.56 (m, 2H), 7.51-7.42 (m, 1H), 5.59-5.24 (m, 1H), 5.06-4.73 (m, 2H), 4.65-4.21 (m, 5H), 3.39 (s, 3H), 3.33 (s, 1H), 3.10-2.89 (m, 3H), 2.70 (s, 1H), 2.22-2.10 (m, 2H), 2.03-1.60 (m, 8H), 1.55-1.48 (m, 9H).

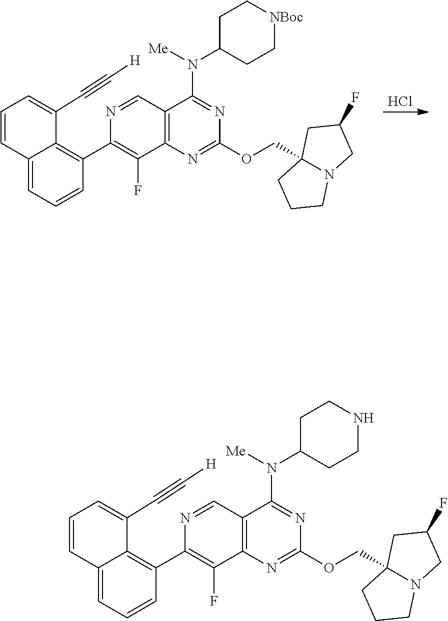 [SMILES: C#Cc1cccc2cccc(-c3ncc4c(N(C)C5CCN(C(=O)OC(C)(C)C)CC5)nc(OC[C@@]56CCCN5C[C@H](F)C6)nc4c3F)c12 ; Cl ; C#Cc1cccc2cccc(-c3ncc4c(N(C)C5CCNCC5)nc(OC[C@@]56CCCN5C[C@H](F)C6)nc4c3F)c12]

### Step 5: Preparation of 7-(8-ethynylnaphthalen-1-yl)-8-fluoro-2-(((2R, 7aS)-2-fluorotetrahydro-H-pyrrolizin-7a(5H)-yl)methoxy)-N-methyl-N-(piperidin-4-yl)pyrido[4,3-d]pyrimidin-4-amine

To a solution of tert-butyl 4-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)piperidine-1-carboxylate (93 mg, 0.139 mmol) in acetonitrile (2 mL) was added a solution of HCl (4M in dioxane, 0.348 mL, 1.391 mmol). The mixture was stirred for 1 h and then made basic by addition of 1-methylimidazole (0.166 mL, 2.086 mmol). The solution was concentrated and used directly in the next step, without further purification (quantitative yield assumed). LC/MS (ESI) m/z: [M+H]⁺ calcd for C₃₃H₃₄F₂N₆O 569.7; found 569.5.

General Scheme for the preparation of the following intermediates:

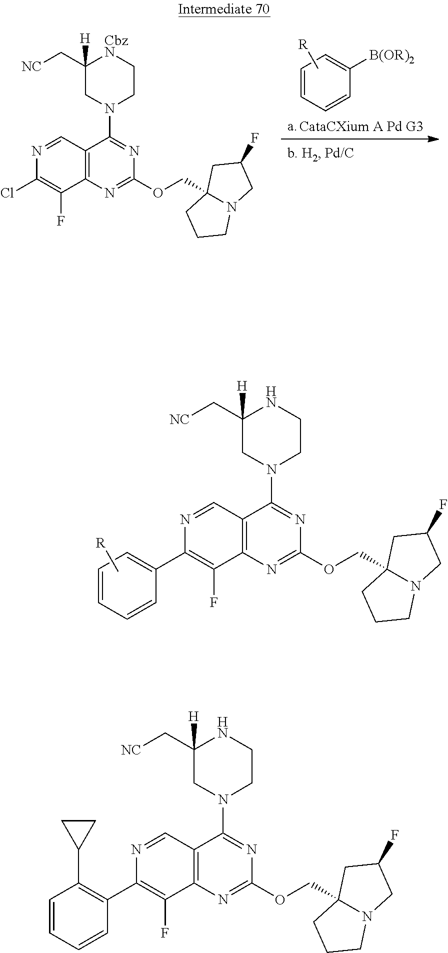

### 2-((S)-4-(7-(2-cyclopropylphenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

### Step 1: Preparation of (S)-2-(cyanomethyl)-4-(7-(2-cyclopropylphenyl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate

To a stirred solution of benzyl (S)-4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-2-(cyanomethyl)piperazine-1-carboxylate (300 mg, 0.502 mmol) in dioxane (3 mL) was added (2-cyclopropylphenyl)boronic acid (163 mg, 1.003 mmol) and potassium phosphate solution (2.0 M, 1.0 mL, 2.007 mmol). Nitrogen was sparged through the solution for 5 min, and CataCXium A Pd G3 (36.5 mg, 0.050 mmol) was added. The reaction mixture was heated at 100° C. for 2h in a microwave reactor. After completion, the reaction mixture was quenched with cold water (50 mL) and extracted with ethyl acetate (3×50 mL). The combined organic layers were dried over sodium sulfate, filtered, and concentrated. The crude residue was purified by column chromatography (35% EtOAc/hexanes) to provide the desired product (200 mg, 0.229 mmol, 45.7% yield) as an off white solid. LC/MS (ESI) m/z: [M+H]⁺ calcd for C₃₈H₄₀F₂N₇O₃ 680.3; found 680.2.

### Step 2: Preparation of 2-((S)-4-(7-(2-cyclopropylphenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

To a stirred solution of benzyl (S)-2-(cyanomethyl)-4-(7-(2-cyclopropylphenyl)-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (200 mg, 0.294 mmol) in ethanol (5 mL), was added Pd/C (157 mg, 0.147 mmol) at room temperature. The resulting suspension was stirred under a hydrogen atmosphere (balloon) 2 h. The reaction mixture was filtered through a pad of diatomaceous earth, eluting with DCM/MeOH (1:1, 20 mL) pad. The filtrate was concentrated, and the crude residue was purified by reverse-phase column chromatography [THF:Water: ACN (50:20:30) as a diluent and 75% ACN in 10 mM ammonium bicarbonate as an eluent] to provide the desired product (80 mg, 0.142 mmol, 48% yield) as an off white solid. LC/MS (ESI) m/z: [M+H]⁺ calcd for C₃₀H₃₄F₂N₇O 546.3; found 546.2; ¹H NMR (400 MHz, DMSO-d₆): δ 9.14 (s, 1H), 7.40-7.31 (m, 3H), 7.04 (d, J=7.60 Hz, 1H), 5.28 (d, J=53.20 Hz, 1H), 4.45 (dd, J=11.60, Hz, 2H), 3.95 (m, 2H), 3.31 (m, 1H), 3.02-3.10 (m, 6H), 2.74-2.77 (m, 5H), 2.05-2.06 (m, 3H), 1.79-1.82 (m, 4H), 0.78 (dd, J=2.00, 4.40 Hz, 2H), 0.63 (dd, J=2.00, 5.00 Hz, 2H).

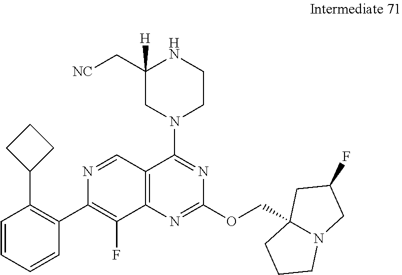 [SMILES: N#CC[C@H]1CN(c2nc(OC[C@@]34CCCN3C[C@H](F)C4)nc3c(F)c(-c4ccccc4C4CCC4)ncc23)CCN1]

### 2-((S)-4-(7-(2-cyclobutylphenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

This intermediate was prepared following the general procedure above using (2-cyclobutylphenyl)boronic acid in place of (2-cyclopropylphenyl)boronic acid. LCMS (ESI) m/z: [M+H]⁺ cacld for C₃₁H₃₆F₂N₇O 560.3; found 560.2; ¹H NMR (400 MHz, DMSO-d₆): 9.11 (s, 1H), 7.50-7.48 (m, 2H), 7.35-7.29 (m, 2H), 5.30-5.20 (m, 1H), 4.45-4.43 (m, 2H), 4.17-4.04 (m, 2H), 3.60 (m, 1H), 3.39 (m, 1H), 3.16-3.02 (m, 6H), 2.88-2.74 (m, 5H), 2.05-1.93 (m, 7H), 1.81-1.68 (m, 5H).

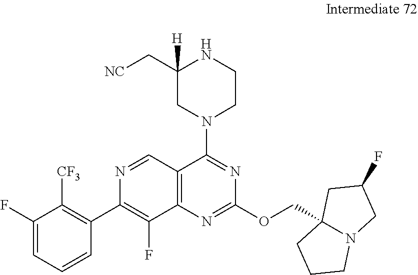 [SMILES: N#CC[C@H]1CN(c2nc(OC[C@@]34CCCN3C[C@H](F)C4)nc3c(F)c(-c4cccc(F)c4C(F)(F)F)ncc23)CCN1]

### 2-((S)-4-(8-fluoro-7-(3-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

This intermediate was prepared following the general procedure above using 3-fluoro-2-(trifluoromethyl)phenylboronic acid in place of (2-cyclopropylphenyl)boronic acid. LCMS (ESI) m/z: [M+H]⁺ cacld for C₂₈H₂₈F₆N₇O 592.2; found 592.2; ¹H NMR (400 MHz, DMSO-d₆): δ 9.11 (s, 1H), 7.87-7.92 (m, 1H), 7.70 (t, J=8.40 Hz, 1H), 7.43 (d, J=7.60 Hz, 1H), 5.27 (d, J=53.60 Hz, 1H), 4.44 (d, J=12.00 Hz, 1H), 4.33 (d, J=13.20 Hz, 1H), 4.15 (d, J=10.40 Hz, 1H), 4.05 (d, J=10.40 Hz, 1H), 3.40 (m, 1H), 3.07-3.17 (m, 6H), 2.74-2.82 (m, 5H), 2.05-2.14 (m, 3H), 1.77-1.85 (m, 3H).

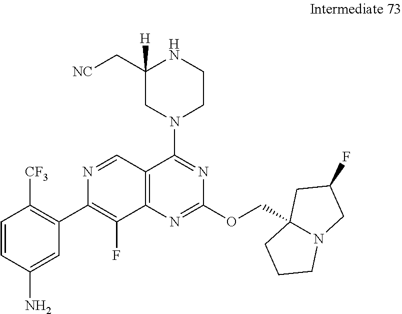 [SMILES: N#CC[C@H]1CN(c2nc(OC[C@@]34CCCN3C[C@H](F)C4)nc3c(F)c(-c4cc(N)ccc4C(F)(F)F)ncc23)CCN1]

### 2-((S)-4-(7-(5-amino-2-(trifluoromethyl)phenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

This intermediate was prepared following the general procedure above using 5-amino-2-(trifluoromethyl)phenylboronic acid in place of (2-cyclopropylphenyl)boronic acid. LCMS (ESI) m/z: [M+H]⁺ cacld for C₂₈H₂₉F₅N₈O 589.2; found 589.2; ¹H NMR (400 MHz, DMSO-d₆): δ 9.07 (s, 1H), 7.47 (d, J=8.40 Hz, 1H), 6.75 (dd, J=2.00, 8.60 Hz, 1H), 6.57 (d, J=2.00 Hz, 1H), 5.99 (s, 2H), 5.27 (d, J=53.60 Hz, 1H), 4.43 (d, J=11.60 Hz, 1H), 4.31 (d, J=11.60 Hz, 1H), 4.10 (m, 2H), 3.39-3.30 (m, 1H), 3.16-3.01 (m, 6H), 2.87-2.74 (m, 5H), 2.15-2.00 (m, 3H), 1.85-1.77 (m, 3H).

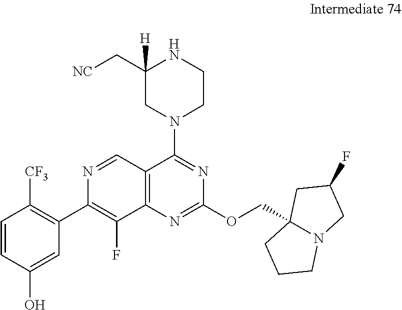 [SMILES: N#CC[C@H]1CN(c2nc(OC[C@@]34CCCN3C[C@H](F)C4)nc3c(F)c(-c4cc(O)ccc4C(F)(F)F)ncc23)CCN1]

### 2-((S)-4-(7-(5-amino-2-(trifluoromethyl)phenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

This intermediate was prepared following the general procedure above using 3-(4,4,5,5-Tetramethyl-1,3,2-dioxaborolan-2-yl)-4-(trifluoromethyl)phenol in place of (2-cyclopropylphenyl)boronic acid. LCMS (ESI) m/z: [M+H]⁺ cacld for C₂₈H₂₉F₅N₇O₂ 590.2; found 590.2.

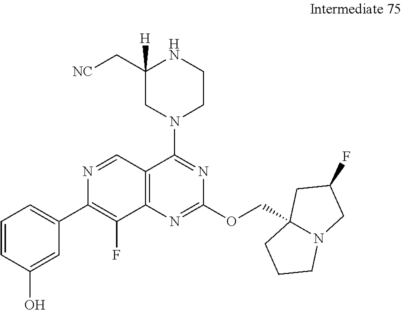 [SMILES: N#CC[C@H]1CN(c2nc(OC[C@@]34CCCN3C[C@H](F)C4)nc3c(F)c(-c4cccc(O)c4)ncc23)CCN1]

### 2-((S)-4-(8-fluoro-2-(((2R, 7aS)-2-fluorotetrahydro-1H-pyrrolizin-7a(5H)-yl)methoxy)-7-(3-hydroxyphenyl)pyrido[4,3-d]pyrimidin-4-yl)piperazin-2-yl)acetonitrile

This intermediate was prepared following the general procedure above using (3-hydroxyphenyl)boronic acid in place of (2-cyclopropylphenyl)boronic acid. LC/MS (ESI) m/z: [M+H]⁺ cacld for C₂₇H₃₀F₂N₇O₂ 522.2; found 522.2.

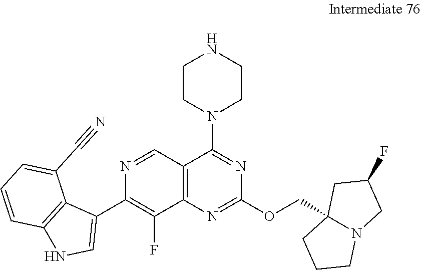 [SMILES: N#Cc1cccc2[nH]cc(-c3ncc4c(N5CCNCC5)nc(OC[C@@]56CCCN5C[C@H](F)C6)nc4c3F)c12]

### 3-(8-fluoro-2-(((2R, 7aS)-2-fluorotetrahydro-1H-pyrrolizin-7a(5H)-methoxy)-4-(piperazin-1-yl)pyrido[4,3-d]pyrimidin-7-yl)-1H-indole-4-carbonitrile

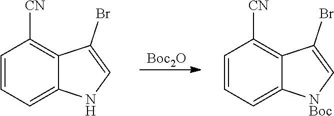 [SMILES: "Boc2O" ; N#Cc1cccc2[nH]cc(Br)c12 ; CC(C)(C)OC(=O)n1cc(Br)c2c(C#N)cccc21]

### Step 1: Preparation of tert-butyl 3-bromo-4-cyano-1H-indole-1-carboxylate

A solution of 3-bromo-1H-indole-4-carbonitrile (200 mg, 0.905 mmol), Et₃N (0.265 mL, 1.90 mmol) and di-tert-butyl dicarbonate (0.231 mL, 0.995 mmol) in DCM (10 mL) was heated at 40° C. for 2 h. The reaction mixture was concentrated, and the crude residue was purified by column chromatography (0→35% EtOAc/hexanes) to provide the desired product (270 mg, 0.84 mmol, 93% yield) as a white solid. LC/MS (ESI) m/z: [M+H]⁺ calcd for C₁₄H₁₄BrN₂O₂ 321.0; found 321.0; ¹H NMR (500 MHz, CDCl₃) δ 8.50 (br d, J=8.5 Hz, 1H), 7.81 (s, 1H), 7.67 (d, J=7.5 Hz, 1H), 7.44 (t, J=7.9 Hz, 1H), 1.70 (s, 9H).

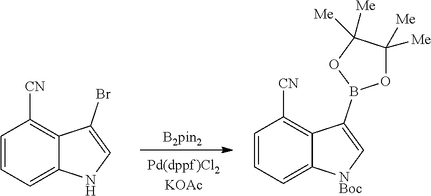 [SMILES: N#Cc1cccc2[nH]cc(Br)c12 ; "B2pin2" ; "Pd(dppf)Cl2KOAc" ; CC(C)(C)OC(=O)n1cc(B2OC(C)(C)C(C)(C)O2)c2c(C#N)cccc21]

### Step 2: Preparation of tert-butyl 4-cyano-3-(4,4,5,5-tetramethyl-1,3,2-dioxaborolan-2-yl)-1H-indole-1-carboxylate

To a suspension of tert-butyl 3-bromo-4-cyano-1H-indole-1-carboxylate (250 mg, 0.778 mmol), bis(pinacolato)diboron (296 mg, 1.168 mmol) and potassium acetate (229 mg, 2.335 mmol) in dioxane (10 mL) was added 1,1′-Bis(diphenylphosphino)ferrocene dichloropalladium (II) (28.5 mg, 0.039 mmol). The reaction vial was sealed then heated at 90° C. for 12 hrs. The reaction mixture was diluted with EtOAc (20 mL) and filtered. The filtrate was concentrated, and the cured residue was purified by column chromatography (5→40% EtOAc/hexanes) to provide the desired product (105 mg, 0.29 mmol, 37% yield) as a clear oil. LC/MS (ESI) m/z: [M+H]⁺ calcd for C₂₀H₂₆BN₂O₄ 369.2; found 369.2; ¹H NMR (500 MHz, CDCl₃) δ 8.48 (d, J=8.6 Hz, 1H), 8.10 (s, 1H), 7.65 (d, J=7.5 Hz, 1H), 7.38 (t, J=8.0 Hz, 1H), 1.70 (s, 9H), 1.45 (s, 12H).

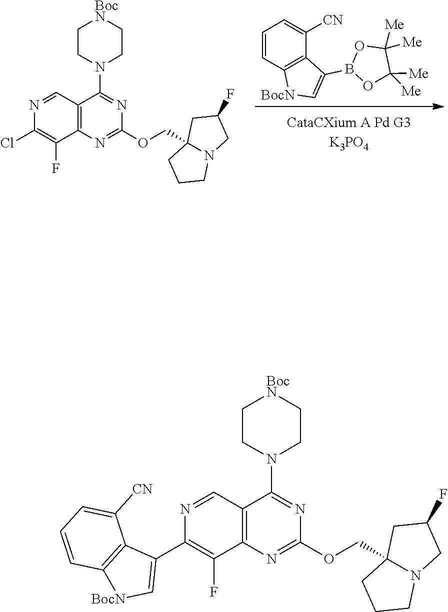 [SMILES: CC(C)(C)OC(=O)N1CCN(c2nc(OC[C@@]34CCCN3C[C@H](F)C4)nc3c(F)c(Cl)ncc23)CC1 ; CC(C)(C)OC(=O)n1cc(B2OC(C)(C)C(C)(C)O2)c2c(C#N)cccc21 ; CC(C)(C)OC(=O)N1CCN(c2nc(OC[C@@]34CCCN3C[C@H](F)C4)nc3c(F)c(-c4cn(C(=O)OC(C)(C)C)c5cccc(C#N)c45)ncc23)CC1]

### Step 3: Preparation of tert-butyl 3-(4-(4-(tert-butoxycarbonyl)piperazin-1-yl)-8-fluoro-2-(((2R, 7aS)-2-fluorotetrahydro-1H-pyrrolizin-7a(5H)-yl)methoxy)pyrido[4,3-d]pyrimidin-7-yl)-4-cyano-1H-indole-1-carboxylate

tert-Butyl 4-(7-chloro-8-fluoro-2-(((2R, 7aS)-2-fluorotetrahydro-1H-pyrrolizin-7a(5H)-yl)methoxy)pyrido[4,3-d]pyrimidin-4-yl)piperazine-1-carboxylate (150 mg, 0.286 mmol), tert-butyl 4-cyano-3-(4,4,5,5-tetramethyl-1,3,2-dioxaborolan-2-yl)-1H-indole-1-carboxylate (105 mg, 0.286 mmol), and methanesulfonato(diadamantyl-n-butylphosphino)-2′-amino-1,1′-biphenyl-2-yl)palladium(II) dichloromethane adduct (10.40 mg, 0.014 mmol) were combined as solids in a sealable reaction vial. The vial was sealed. The atmosphere was evacuated and replaced with nitrogen. Degassed dioxane (2 ml) and potassium phosphate solution (2.0 M in water, 0.43 ml, 0.857 mmol) were added, and the reaction mixture was heated at 100° C. for 2 h. The reaction mixture was diluted with EtOAc (10 mL), filtered, and concentrated. The crude residue was purified by column chromatography (0→100% EtOAc/hexanes) to provide the desired product (90 mg, 0.13 mmol, 44% yield) as a white wax. LC/MS (ESI) m/z: [M+H]⁺ calcd for C₃₈H₄₅F₂N₈O_{5 731.8}; found 731.5; ¹H NMR (500 MHz, CD₃OD) δ 9.17 (s, 1H), 8.63 (d, J=8.5 Hz, 1H), 8.22 (s, 1H), 7.74 (d, J=7.5 Hz, 1H), 7.57 (t, J=7.9 Hz, 1H), 5.32 (d, J=52.2 Hz, 1H), 4.84-4.80 (m, 1H), 4.36-4.23 (m, 2H), 4.16-4.07 (m, 3H), 3.77-3.67 (m, 3H), 3.31-3.17 (m, 4H), 3.06-2.99 (m, 2H), 2.34 (d, J=4.4 Hz, 1H), 2.30-2.19 (m, 2H), 2.15 (br d, J=8.8 Hz, 1H), 1.95-1.87 (m, 1H), 1.75 (s, 9H), 1.54-1.51 (m, 9H).

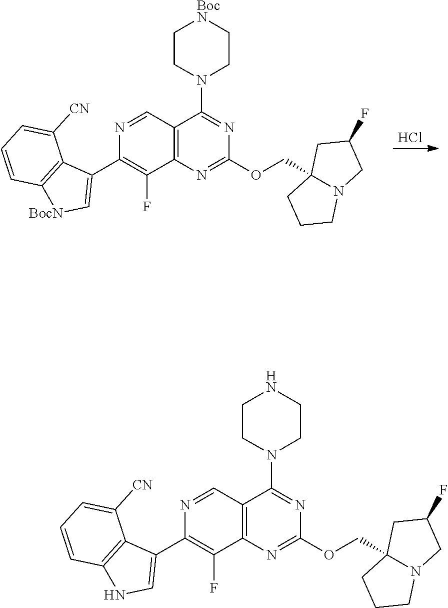 [SMILES: CC(C)(C)OC(=O)N1CCN(c2nc(OC[C@@]34CCCN3C[C@H](F)C4)nc3c(F)c(-c4cn(C(=O)OC(C)(C)C)c5cccc(C#N)c45)ncc23)CC1 ; Cl ; N#Cc1cccc2[nH]cc(-c3ncc4c(N5CCNCC5)nc(OC[C@@]56CCCN5C[C@H](F)C6)nc4c3F)c12]

### Step 4: Preparation of 3-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)-1H-indole-4-carbonitrile

To a room temperature solution of tert-butyl 3-(4-(4-(tert-butoxycarbonyl)piperazin-1-yl)-8-fluoro-2-(((2R, 7aS)-2-fluorotetrahydro-1H-pyrrolizin-7a(5H)-yl)methoxy)pyrido[4,3-d]pyrimidin-7-yl)-4-cyano-1H-indole-1-carboxylate (85 mg, 0.116 mmol) in dioxane (1 mL) was added a solution of HCl (4.0 M in dioxane, 0.3 mL, 1.200 mmol) drop wise. After 7 h, the reaction mixture was concentrated. The crude residue was used directly in the next steps, without additional purification (quantitative yield assumed). LC/MS (ESI) m/z: [M+H]⁺ calcd for C₂₈H₂₉F₂N₈O 531.6; found 531.3.

### Preparation of KRAS Inhibitors

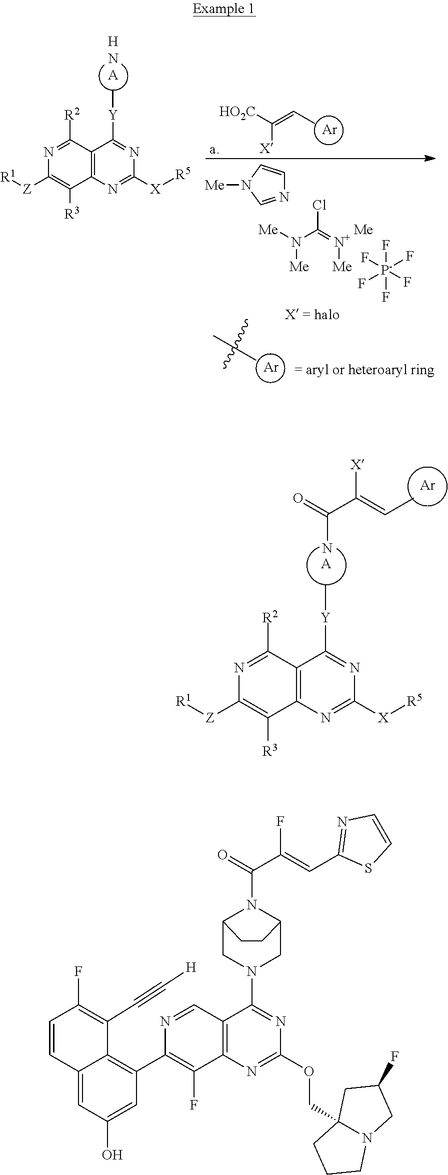

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(thiazol-2-yl)prop-2-en-1-one

4-(4-((1R, 5S)-3,8-Diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (Intermediate 1, 8.6 mg, 0.014 mmol) and (Z)-2-fluoro-3-(thiazol-2-yl)acrylic acid (12.4 mg, 0.072 mmol) were combined as solids and dissolved in DMF (700 μL). 1-Methylimidazole (11 μL, 0.14 mmol) was added followed by chloro-N,N,N′,N′-tetramethylformamidinium hexafluorophosphate (20 mg, 0.072 mmol). The reaction mixture was stirred for 5 min. A saturated aqueous solution of ammonium hydroxide (100 μL) was added and the reaction mixture was stirred for an additional 5 min. The reaction mixture was directly purified by reverse phase HPLC (column: Xbridge C_{18, 19} mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (8.9 mg, 0.012 mmol, 82% yield) as a tan solid. LC/MS (ESI) m/z: [M+H]⁺ calcd for C₃₉H₃₄F₄N₇O₃S 756.2; found 756.0; ¹H NMR (500 MHz, DMSO-d₆) δ 10.33 (br s, 1H), 9.08 (s, 1H), 8.05-8.01 (m, 2H), 7.98 (dd, J=9.2, 5.9 Hz, 1H), 7.46 (t, J=9.0 Hz, 1H), 7.40 (d, J=2.6 Hz, 1H), 7.22 (d, J=33.9 Hz, 1H), 7.18 (s, 1H), 5.37-5.20 (m, 1H), 4.84 (br s, 2H), 4.70 (br d, J=12.3 Hz, 1H), 4.54 (br d, J=12.3 Hz, 1H), 4.14 (dd, J=10.4, 2.3 Hz, 1H), 4.05 (br d, J=10.4 Hz, 1H), 3.94 (s, 1H), 3.90-3.70 (m, 2H), 3.15-3.01 (m, 3H), 2.87-2.80 (m, 1H), 2.18-1.90 (m, 5H), 1.89-1.74 (m, 5H).

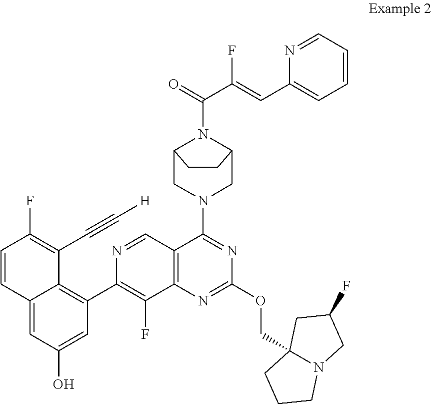 [SMILES: C#Cc1c(F)ccc2cc(O)cc(-c3ncc4c(N5CC6CCC(C5)N6C(=O)/C(F)=C/c5ccccn5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(pyridin-2-yl)prop-2-en-1-one

4-(4-((1R, 5S)-3,8-Diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (Intermediate 1A, 5.0 mg, 0.008 mmol) and (Z)-2-fluoro-3-(pyridin-2-yl)acrylic acid (5.6 mg, 0.033 mmol) were combined as solids and dissolved in DMF (400 μL). 1-methylimidazole (5 μL, 0.067 mmol) was added followed by chloro-N,N,N′,N′-tetramethylformamidinium hexafluorophosphate (9.3 mg, 0.033 mmol). The reaction mixture was stirred for 5 min. A saturated aqueous solution of ammonium hydroxide (100 μL) was added and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (5.9 mg, 7.87 μmol, 95% yield) as an off-white solid. LC/MS (ESI) m/z: [M+H]⁺ calcd for C₄₁H₃₆F₄N₇O₃ 750.2; found 750.0; ¹H NMR: (500 MHz, DMSO-d₆) δ 10.20 (br s, 1H), 9.08 (s, 1H), 8.66 (br d, J=4.7 Hz, 1H), 7.97 (dd, J=9.1, 5.8 Hz, 1H), 7.90 (t, J=7.7 Hz, 1H), 7.82 (d, J=7.9 Hz, 1H), 7.46 (t, J=9.0 Hz, 1H), 7.41-7.36 (m, 2H), 7.22-7.13 (m, 1H), 6.82 (d, J=37.9 Hz, 1H), 5.38-5.19 (m, 1H), 4.70 (br d, J=12.0 Hz, 1H), 4.54 (br d, J=12.0 Hz, 1H), 4.59-4.46 (m, 1H), 4.15 (br d, J=10.3 Hz, 1H), 4.07 (br d, J=10.3 Hz, 1H), 3.94 (s, 1H), 3.86-3.73 (m, 2H), 3.17-3.07 (m, 2H), 3.14-3.01 (m, 3H), 2.89-2.79 (m, 1H), 2.19-1.93 (m, 5H), 1.88-1.70 (m, 5H).

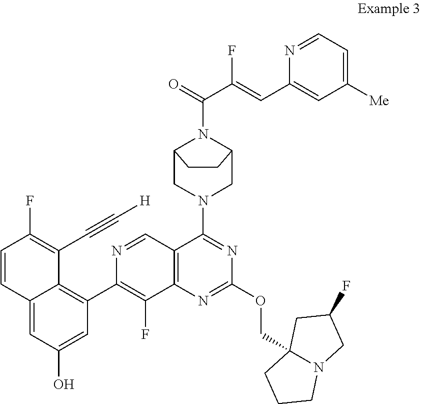 [SMILES: C#Cc1c(F)ccc2cc(O)cc(-c3ncc4c(N5CC6CCC(C5)N6C(=O)/C(F)=C/c5cc(C)ccn5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(4-methylpyridin-2-yl)prop-2-en-1-one

4-(4-((1R, 5S)-3,8-Diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (Intermediate 1A, 5.0 mg, 0.008 mmol) and (Z)-2-fluoro-3-(4-methylpyridin-2-yl)acrylic acid (6.0 mg, 0.033 mmol) were combined as solids and dissolved in DMF (400 μL). 1-methylimidazole (5 μL, 0.067 mmol) was added followed by chloro-N,N,N′,N′-tetramethylformamidinium hexafluorophosphate (9.3 mg, 0.033 mmol). The reaction mixture was stirred for 5 min. A saturated aqueous solution of ammonium hydroxide (100 μL) was added and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 72% 5:95 MeCN:H₂O with 10 mM AA/28% 95:5 MeCN:H₂O with 10 mM AA→100% 95:5 MeCN:H₂O with 10 mM AA; λ=220 nm) to provide the desired product (3.6 mg, 4.71 μmol, 57% yield) as a brown solid. LC/MS (ESI) m/z: [M+H]⁺ calcd for C₄₂H₃₈F₄N₇O₃ 764.3; found 764.0; ¹H NMR: (500 MHz, DMSO-d₆) δ 10.22 (br s, 1H) 9.08 (s, 1H), 8.51 (d, J=4.9 Hz, 1H), 7.98 (dd, J=9.2, 6.0 Hz, 1H), 7.66 (s, 1H), 7.46 (t, J=9.0 Hz, 1H), 7.40 (d, J=2.5 Hz, 1H), 7.22 (d, J=4.5 Hz, 1H), 7.18 (s, 1H), 6.78 (d, J=38.0 Hz, 1H), 5.43-5.17 (m, 1H), 4.83 (br s, 2H), 4.70 (br d, J=11.0 Hz, 1H), 4.53 (br d, J=11.0 Hz, 1H), 4.25-4.13 (m, 1H), 4.12-4.04 (m, 1H), 3.93 (s, 1H), 3.86-3.72 (m, 2H), 3.23-3.05 (m, 3H), 2.94-2.81 (m, 1H), 2.37 (s, 3H), 2.21-1.93 (m, 5H), 1.89-1.68 (m, 5H).

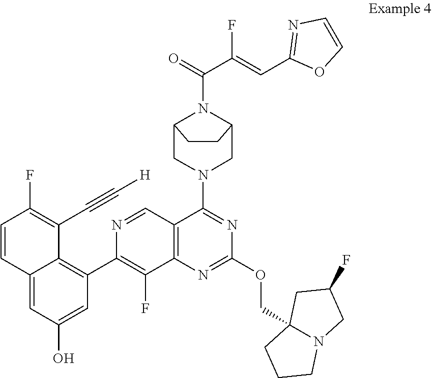 [SMILES: C#Cc1c(F)ccc2cc(O)cc(-c3ncc4c(N5CC6CCC(C5)N6C(=O)/C(F)=C/c5ncco5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(oxazol-2-yl)prop-2-en-1-one

4-(4-((1R, 5S)-3,8-Diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (5.0 mg, 0.008 mmol) and (Z)-2-fluoro-3-(oxazol-2-yl)acrylic acid (5.2 mg, 0.033 mmol) were combined as solids and dissolved in DMF (400 L). 1-methylimidazole (5 μL, 0.067 mmol) was added followed by chloro-N,N,N′,N′-tetramethylformamidinium hexafluorophosphate (9.3 mg, 0.033 mmol). The reaction mixture was stirred for 5 min. A saturated aqueous solution of ammonium hydroxide (100 L) was added and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 78% 5:95 MeCN:H₂O with 10 mM AA/22% 95:5 MeCN:H₂O with 10 mM AA→100% 95:5 MeCN:H₂O with 10 mM AA; λ=220 nm) to provide (4.1 mg, 5.54 μmol, 67% yield) as a brown solid.LC/MS (ESI) m/z: [M+H]⁺ calcd for C₃₉H₃₄F₄N₇O₄ 740.3; found 740.0; ¹H NMR: (500 MHz, DMSO-d₆) δ 10.21 (br s, 1H), 9.07 (s, 1H), 8.31-8.28 (m, 1H), 7.97 (dd, J=9.2, 6.1 Hz, 1H), 7.49-7.43 (m, 2H), 7.40 (d, J=2.3 Hz, 1H), 7.18 (s, 1H), 6.81 (d, J=34.7 Hz, 1H), 5.37-5.19 (m, 1H), 4.88-4.77 (m, 2H), 4.69 (br d, J=12.0 Hz, 1H), 4.53 (br d, J=12.0 Hz, 1H), 4.15 (br d, J=10.1 Hz, 1H), 4.06 (br d, J=10.1 Hz, 1H), 3.93 (s, 1H), 3.89-3.69 (m, 2H), 3.18-3.02 (m, 3H) 2.87-2.81 (m, 1H), 2.18-1.91 (m, 5H), 1.89-1.73 (m, 5H).

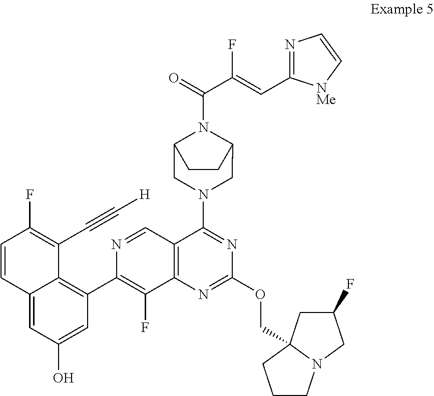 [SMILES: C#Cc1c(F)ccc2cc(O)cc(-c3ncc4c(N5CC6CCC(C5)N6C(=O)/C(F)=C/c5nccn5C)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(1-methyl-1H-imidazol-2-yl)prop-2-en-1-one

4-(4-((1R, 5S)-3,8-Diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (5.0 mg, 0.008 mmol) and (Z)-2-fluoro-3-(1-methyl-1H-imidazol-2-yl)acrylic acid (5.7 mg, 0.033 mmol) were combined as solids and dissolved in DMF (400 μL). 1-methylimidazole (5 μL, 0.067 mmol) was added followed by chloro-N,N,N′,N′-tetramethylformamidinium hexafluorophosphate (9.3 mg, 0.033 mmol). The reaction mixture was stirred for 5 min. A saturated aqueous solution of ammonium hydroxide (100 μL) was added and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 82% 5:95 MeCN:H₂O with 10 mM AA/18% 95:5 MeCN:H₂O with 10 mM AA→100% 95:5 MeCN:H₂O with 10 mM AA; λ=220 nm) to provide the desired product (4.1 mg, 5.45 μmol, 65% yield) as a brown solid. LC/MS (ESI) m/z: [M+H]⁺ calcd for C₄₀H₃₇F₄N₈O₃ 753.3; found 753.0; ¹H NMR: (500 MHz, DMSO-d₆) δ 10.22 (br s, 1H), 9.07 (s, 1H), 7.97 (dd, J=9.2, 5.9 Hz, 1H), 7.46 (t, J=8.9 Hz, 1H), 7.39 (d, J=2.5 Hz, 1H), 7.29 (s, 1H), 7.18 (s, 1H), 7.09 (s, 1H), 6.80 (d, J=34.4 Hz, 1H), 5.37-5.20 (m, 1H), 4.86-4.78 (m, 2H), 4.69 (br d, J=12.3 Hz, 1H), 4.54 (br d, J=12.3 Hz, 1H), 4.14 (dd, J=10.4, 2.2 Hz, 1H), 4.05 (br d, J=10.4 Hz, 1H), 3.93 (s, 1H), 3.83-3.75 (m, 2H), 3.74 (s, 3H), 3.15-3.01 (m, 3H), 2.87-2.79 (m, 1H), 2.18-1.92 (m, 4H), 1.90-1.72 (m, 6H).

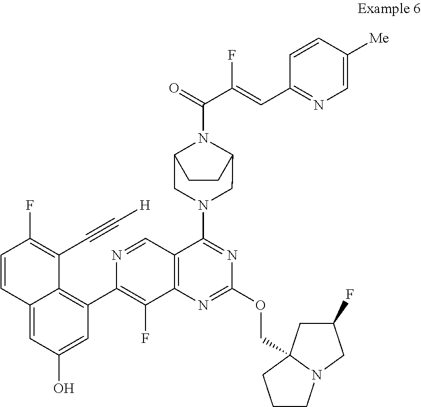 [SMILES: C#Cc1c(F)ccc2cc(O)cc(-c3ncc4c(N5CC6CCC(C5)N6C(=O)/C(F)=C/c5ccc(C)cn5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(5-methylpyridin-2-yl)prop-2-en-1-one

4-(4-((1R, 5S)-3,8-Diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (5.0 mg, 0.008 mmol) and (Z)-2-fluoro-3-(5-methylpyridin-2-yl)acrylic acid (6.0 mg, 0.033 mmol) were combined as solids and dissolved in DMF (400 μL). 1-methylimidazole (5 μL, 0.067 mmol) was added followed by chloro-N,N,N′,N′-tetramethylformamidinium hexafluorophosphate (9.3 mg, 0.033 mmol). The reaction mixture was stirred for 5 min. A saturated aqueous solution of ammonium hydroxide (100 μL) was added and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 78% 5:95 MeCN:H₂O with 10 mM AA/22% 95:5 MeCN:H₂O with 10 mM AA→100% 95:5 MeCN:H₂O with 10 mM AA; λ=220 nm) to provide the desired product (4.1 mg, 5.37 μmol, 64% yield) as a brown solid. LC/MS (ESI) m/z: [M+H]⁺ calcd for C₄₂H₃₈F₄N₇O₃ 764.3; found 764.0; ¹H NMR (500 MHz, DMSO-d₆) δ 10.19 (br s, 1H), 9.08 (s, 1H), 8.50 (s, 1H), 7.97 (dd, J=9.2, 6.0 Hz, 1H), 7.72 (s, 2H), 7.46 (t, J=9.0 Hz, 1H), 7.39 (d, J=2.4 Hz, 1H), 7.18 (s, 1H), 6.80 (d, J=38.2 Hz, 1H), 5.39-5.21 (m, 1H), 4.82 (br s, 2H), 4.69 (br d, J=12.5 Hz, 1H), 4.53 (br d, J=12.5 Hz, 1H), 4.23-4.13 (m, 1H), 4.13-4.02 (m, 1H), 3.93 (s, 1H), 3.86-3.69 (m, 2H), 3.15-3.01 (m, 3H), 2.92-2.78 (m, 1H), 2.33 (s, 3H), 2.22-1.92 (m, 5H), 1.90-1.74 (m, 5H).

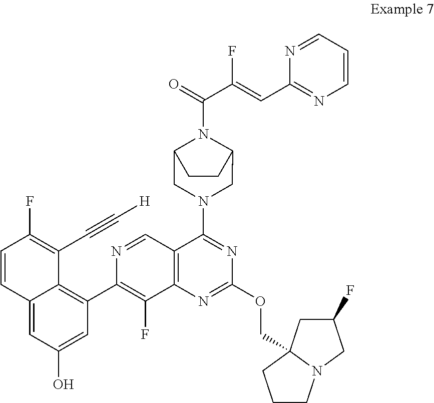 [SMILES: C#Cc1c(F)ccc2cc(O)cc(-c3ncc4c(N5CC6CCC(C5)N6C(=O)/C(F)=C/c5ncccn5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(pyrimidin-yl)prop-2-en-1-one

4-(4-((1R, 5S)-3,8-Diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (5.0 mg, 0.008 mmol) and (Z)-2-fluoro-3-(pyrimidin-2-yl)acrylic acid (5.7 mg, 0.033 mmol) were combined as solids and dissolved in DMF (400 L). 1-methylimidazole (5 μL, 0.067 mmol) was added followed by chloro-N,N,N′,N′-tetramethylformamidinium hexafluorophosphate (9.3 mg, 0.033 mmol). The reaction mixture was stirred for 5 min. A saturated aqueous solution of ammonium hydroxide (100 L) was added and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 85% 5:95 MeCN:H₂O with 10 mM AA/15% 95:5 MeCN:H₂O with 10 mM AA→100% 95:5 MeCN:H₂O with 10 mM AA; λ=220 nm) to provide the desired product (3.8 mg, 5.06 μmol, 61% yield) as a brown solid. LC/MS (ESI) m/z: [M+H]⁺ calcd for C₄₀H₃₅F₄N₈O_{3 751.3}; found 751.0; ¹H NMR (500 MHz, DMSO-d₆) δ 10.17 (br s, 1H), 9.07 (s, 1H), 8.90 (d, J=4.9 Hz, 2H), 7.97 (dd, J=9.1, 6.0 Hz, 1H), 7.49-7.43 (m, 2H), 7.40 (d, J=2.4 Hz, 1H), 7.18 (s, 1H), 6.79 (d, J=34.2 Hz, 1H), 5.43-5.18 (m, 1H), 4.82 (br s, 2H), 4.69 (br d, J=12.3 Hz, 1H), 4.54 (br d, J=13.3 Hz, 1H), 4.16 (br d, J=9.6 Hz, 1H), 4.07 (br d, J=9.6 Hz, 1H), 3.93 (s, 1H), 3.87-3.72 (m, 2H), 3.14-3.01 (m, 3H), 2.90-2.80 (m, 1H), 2.18-1.92 (m, 5H), 1.90-1.74 (m, 5H).

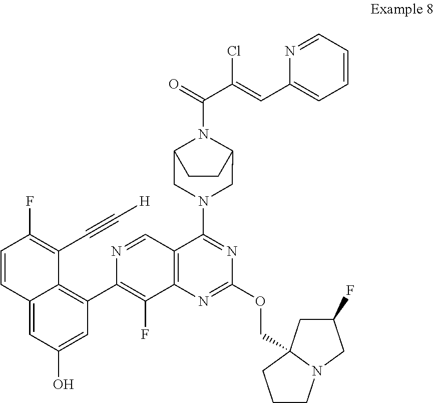 [SMILES: C#Cc1c(F)ccc2cc(O)cc(-c3ncc4c(N5CC6CCC(C5)N6C(=O)/C(Cl)=C/c5ccccn5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.I]octan-8-yl)-2-chloro-3-(pyridin-yl)prop-2-en-1-one

4-(4-((1R, 5S)-3,8-diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (5.0 mg, 0.008 mmol) and (Z)-2-chloro-3-(pyridin-2-yl)acrylic acid (4.6 mg, 0.025 mmol) were combined as solids and dissolved in DMF (400 L). 1-methylimidazole (4 μL, 0.050 mmol) was added followed by chloro-N,N,N′,N′-tetramethylformamidinium hexafluorophosphate (7.0 mg, 0.025 mmol). The reaction mixture was stirred for 5 min. A saturated aqueous solution of ammonium hydroxide (50 L) was added and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN: H₂O with 10 mM AA→100% 95:5 MeCN: H₂O with 10 mM AA; λ=220 nm) to provide the desired product (5.5 mg, 7.18 μmol, 86% yield) as a brown solid. LC/MS (ESI) m/z: [M+H]⁺ calcd for C₄₁H₃₆CIF₃N₇O₃ 766.3; found 766.2; ¹H NMR (500 MHz, DMSO-d₆) δ 10.18 (s, 1H), 9.10 (s, 1H), 8.70-8.67 (m, J=4.7 Hz, 1H), 8.00-7.89 (m, 3H), 7.46 (t, J=9.2 Hz, 1H), 7.43-7.38 (m, 2H), 7.31 (s, 1H), 7.18 (s, 1H), 5.46-5.26 (m, 1H), 4.82-4.45 (m, 4H), 4.36-4.04 (m, 2H), 3.91 (br s, 1H), 3.88-3.65 (m, 2H), 3.23-3.08 (m, 3H), 3.01-2.85 (m, 1H), 2.32-1.92 (m, 6H), 1.90-1.77 (m, 4H).

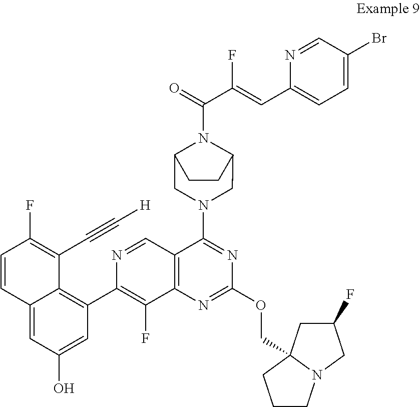 [SMILES: C#Cc1c(F)ccc2cc(O)cc(-c3ncc4c(N5CC6CCC(C5)N6C(=O)/C(F)=C/c5ccc(Br)cn5)nc(OC[C@@]56CCCN5C[C@H](F)C6)nc4c3F)c12]

### (Z)-3-(5-bromopyridin-2-yl)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoroprop-2-en-1-one

4-(4-((1R, 5S)-3,8-diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (7.0 mg, 0.012 mmol) and (Z)-3-(5-bromopyridin-2-yl)-2-fluoroacrylic acid (5.7 mg, 0.023 mmol) were combined as solids and dissolved in DMF (500 μL). 1-methylimidazole (9 μL, 0.117 mmol) was added followed by chloro-N,N,N′,N′-tetramethylformamidinium hexafluorophosphate (6.5 mg, 0.023 mmol). The reaction mixture was stirred for 5 min. A saturated aqueous solution of ammonium hydroxide (100 μL) was added and the reaction mixture was stirred for an additional 5 min. The reaction mixture was directly purified by reverse phase HPLC (column: Xbridge C_{18, 19} mm×200 mm, 5 m particles; flow rate: 20 mL/min; column temperature: 25° C.; gradient: 71% 5:95 MeCN: H₂O with 10 mM AA/29% 95:5 MeCN: H₂O with 10 mM AA→100% 95:5 MeCN: H₂O with 10 mM AA; λ=220 nm) to provide the desired product (5.4 mg, 6.5 μmol, 56% yield) as a brown solid. LC/MS (ESI) m/z: [M+H]⁺ calcd for C₄₁H₃₅BrF4N₇O₃ 828.2; found 828.1; ¹H NMR (500 MHz, DMSO-d₆) δ 10.18 (br s, 1H), 9.07 (s, 1H), 8.79 (d, J=1.6 Hz, 1H), 8.17 (dd, J=8.4, 2.1 Hz, 1H), 7.97 (dd, J=9.2, 5.8 Hz, 1H), 7.77 (d, J=8.4 Hz, 1H), 7.46 (t, J=9.0 Hz, 1H), 7.39 (d, J=2.4 Hz, 1H), 7.18 (br s, 1H), 6.82 (d, J=37.2 Hz, 1H), 5.41-5.21 (m, 1H), 4.86-4.77 (m, 2H), 4.69 (br d, J=12.7 Hz, 1H), 4.53 (br d, J=12.7 Hz, 1H), 4.25-4.03 (m, 2H), 3.93 (s, 1H), 3.86-3.71 (m, 2H), 3.22-3.06 (m, 3H), 2.94-2.84 (m, 1H), 2.21-1.92 (m, 5H), 1.90-1.78 (m, 5H).

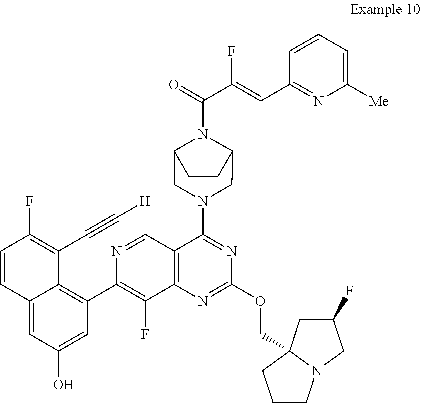 [SMILES: C#Cc1c(F)ccc2cc(O)cc(-c3ncc4c(N5CC6CCC(C5)N6C(=O)/C(F)=C/c5cccc(C)n5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(5-methylpyridin-2-yl)prop-2-en-1-one

4-(4-((1R, 5S)-3,8-diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (5.0 mg, 0.008 mmol) and (Z)-2-fluoro-3-(6-methylpyridin-2-yl)acrylic acid (3.1 mg, 0.017 mmol) were combined as solids and dissolved in DMF (500 μL). 1-methylimidazole (7 μL, 0.083 mmol) was added followed by chloro-N,N,N′,N′-tetramethylformamidinium hexafluorophosphate (4.7 mg, 0.017 mmol). The reaction mixture was stirred for 5 min. A saturated aqueous solution of ammonium hydroxide (100 μL) was added and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 75% 5:95 MeCN: H₂O with 10 mM AA/25% 95:5 MeCN: H₂O with 10 mM AA→100% 95:5 MeCN: H₂O with 10 mM AA; λ=220 nm) to provide the desired product (5.3 mg, 6.9 μmol, 83% yield) as a brown solid. LC/MS (ESI) m/z: [M+H]⁺ calcd for C₄₂H₃₈F₄N₇O₃ 764.3; found 764.1; ¹H NMR (500 MHz, DMSO-d₆) δ 10.19 (br s, 1H), 9.07 (s, 1H), 8.00-7.93 (m, 1H), 7.78 (dd, J=7.9, 7.6 Hz, 1H), 7.62 (d, J=7.9 Hz, 1H), 7.46 (t, J=9.0 Hz, 1H), 7.42-7.37 (m, 1H), 7.25 (d, J=7.6 Hz, 1H), 7.18 (s, 1H), 6.76 (d, J=38.0 Hz, 1H), 5.37-5.18 (m, 1H), 4.88-4.77 (m, 2H), 4.68 (br d, J=12.2 Hz, 1H), 4.52 (d, J=12.2 Hz, 1H), 4.14 (dd, J=10.5, 2.2 Hz, 1H), 4.05 (br d, J=10.5 Hz, 1H), 3.93 (s, 1H), 3.86-3.72 (m, 2H), 3.18-3.03 (m, 3H), 2.87-2.78 (m, 1H), 2.49 (br s, 3H), 2.15-1.91 (m, 5H), 1.89-1.74 (m, 5H).

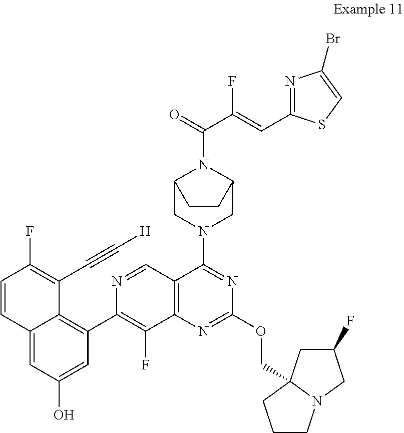 [SMILES: C#Cc1c(F)ccc2cc(O)cc(-c3ncc4c(N5CC6CCC(C5)N6C(=O)/C(F)=C/c5nc(Br)cs5)nc(OC[C@@]56CCCN5C[C@H](F)C6)nc4c3F)c12]

### (Z)-3-(4-bromothiazol-2-yl)-1-((JR, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoroprop-2-en-1-one

4-(4-((1R, 5S)-3,8-diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (5.0 mg, 0.008 mmol) and (Z)-3-(4-bromothiazol-2-yl)-2-fluoroacrylic acid (4.2 mg, 0.017 mmol) were combined as solids and dissolved in DMF (500 μL). 1-methylimidazole (7 μL, 0.083 mmol) was added followed by chloro-N,N,N′,N′-tetramethylformamidinium hexafluorophosphate (4.7 mg, 0.017 mmol). The reaction mixture was stirred for 5 min. A saturated aqueous solution of ammonium hydroxide (100 μL) was added and the reaction mixture was stirred for an additional 5 min. The reaction mixture was directly purified by reverse phase HPLC (column: Xbridge C_{18, 19} mm×200 mm, 5 m particles; flow rate: 20 mL/min; column temperature: 25° C.; gradient: 70% 5:95 MeCN: H₂O with 10 mM AA/30% 95:5 MeCN: H₂O with 10 mM AA→100% 95:5 MeCN: H₂O with 10 mM AA; λ=220 nm) to provide the desired product (4.7 mg, 6.9 μmol, 83% yield) as a brown solid. LC/MS (ESI) m/z: [M+H]⁺ calcd for C₃₉H₃₃BrF₄N₇O₃S 834.1; found 833.9; ¹H NMR (500 MHz, DMSO-d₆) δ 9.08 (s, 1H), 8.11 (s, 1H), 7.98 (dd, J=9.2, 5.9 Hz, 1H), 7.46 (t, J=9.0 Hz, 1H), 7.40 (d, J=2.4 Hz, 1H), 7.18 (br s, 1H), 7.20 (d, J=36.4 Hz, 1H), 5.41-5.21 (m, 1H), 4.86-4.78 (m, 2H), 4.73-4.64 (m, 1H), 4.58-4.48 (m, 1H), 4.23-4.14 (m, 1H), 4.13-4.01 (m, 1H), 3.93 (s, 1H), 3.88-3.67 (m, 2H), 3.21-3.02 (m, 3H), 2.94-2.81 (m, 1H), 2.23-1.91 (m, 5H), 1.90-1.74 (m, 5H).

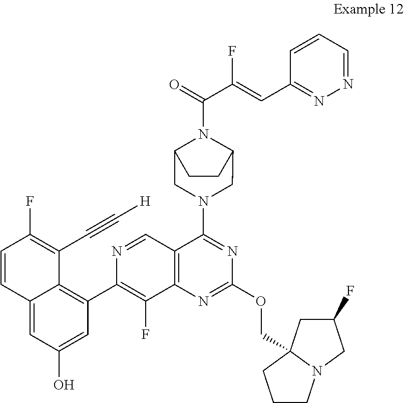 [SMILES: C#Cc1c(F)ccc2cc(O)cc(-c3ncc4c(N5CC6CCC(C5)N6C(=O)/C(F)=C/c5cccnn5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(pyridazin-3-yl)prop-2-en-1-one

4-(4-((1R, 5S)-3,8-diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (5.0 mg, 0.008 mmol) and (Z)-2-fluoro-3-(6-methylpyridin-2-yl)acrylic acid (2.8 mg, 0.017 mmol) were combined as solids and dissolved in DMF (500 μL). 1-methylimidazole (7 μL, 0.083 mmol) was added followed by chloro-N,N,N′,N′-tetramethylformamidinium hexafluorophosphate (4.7 mg, 0.017 mmol). The reaction mixture was stirred for 5 min. A saturated aqueous solution of ammonium hydroxide (100 μL) was added and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 84% 5:95 MeCN:H H₂O with 10 mM AA/16% 95:5 MeCN: H₂O with 10 mM AA→100% 95:5 MeCN: H₂O with 10 mM AA; λ=220 nm) to provide the desired product (4.7 mg, 6.3 μmol, 75% yield) as a brown solid. LC/MS (ESI) m/z: [M+H]⁺ calcd for C₄₀H₃₅F₄N₈O₃ 751.3; found 751.2; ¹H NMR (500 MHz, DMSO-d₆) δ 10.18 (s, 1H), 9.23-9.18 (m, 1H), 9.09-9.04 (m, 1H), 8.07 (dd, J=8.7, 1.5 Hz, 1H), 8.00-7.93 (m, 1H), 7.84-7.76 (m, 1H), 7.46 (t, J=9.1 Hz, 1H), 7.39 (s, 1H), 7.18 (s, 1H), 7.06 (d, J=37.3 Hz, 1H), 5.38-5.20 (m, 1H), 4.88-4.80 (m, 2H), 4.74-4.66 (m, 1H), 4.58-4.49 (m, 1H), 4.16 (br d, J=10.3 Hz, 1H), 4.06 (br d, J=10.3 Hz, 1H), 3.93 (br s, 1H), 3.86 -3.72 (m, 2H), 3.16-3.02 (m, 3H), 2.89-2.80 (m, 1H), 2.18-1.92 (m, 5H), 1.89-1.74 (m, 5H).

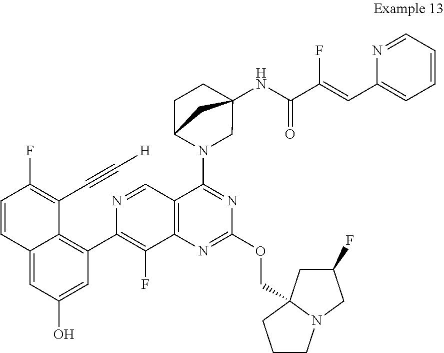 [SMILES: C#Cc1c(F)ccc2cc(O)cc(-c3ncc4c(N5C[C@@]6(NC(=O)/C(F)=C/c7ccccn7)CC[C@@H]5C6)nc(OC[C@@]56CCCN5C[C@H](F)C6)nc4c3F)c12]

### (Z)—N-((1R, 4R)-2-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-2-azabicyclo[2.2.1]heptan-4-yl)-2-fluoro-3-(pyridin-2-yl)acrylamide

4-(4-((1R, 4R)-4-amino-2-azabicyclo[2.2.1]heptan-2-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (3 mg, 0.005 mmol) and (Z)-2-fluoro-3-(pyridin-2-yl)acrylic acid (2.5 mg, 0.015 mmol) were combined as solids and dissolved in DMF (300 L). 1-methylimidazole (2 μL, 0.030 mmol) was added followed by chloro-N,N,N′,N′-tetramethylformamidinium hexafluorophosphate (4.2 mg, 0.015 mmol). The reaction mixture was stirred for 5 min. A saturated aqueous solution of ammonium hydroxide (50 L) was added and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 81% 5:95 MeCN: H₂O with 10 mM AA/19% 95:5 MeCN: H₂O with 10 mM AA→100% 95:5 MeCN: H₂O with 10 mM AA; λ=220 nm) to provide the desired product (1.2 mg, 1.6 μmol, 32% yield) as a brown solid. LC/MS (ESI) m/z: [M+H]⁺ calcd for C₄₁H₃₆F₄N₇O₃ 750.3; found 750.0; ¹H NMR (500 MHz, DMSO-d₆) δ 10.21-10.12 (m, 1H), 9.28-9.22 (m, 1H), 9.18-8.99 (m, 1H), 8.71-8.60 (m, 1H), 8.01-7.95 (m, 1H), 7.93-7.87 (m, 1H), 7.82-7.77 (m, 1H), 7.50-7.43 (m, 1H), 7.41-7.36 (m, 2H), 7.22-7.12 (m, 1H), 6.95 (d, J=36.8 Hz, 1H), 5.47-5.23 (m, 1H), 5.09-4.89 (m, 1H), 4.46-4.31 (m, 1H), 4.28-4.00 (m, 3H), 2.32-1.92 (m, 10H), 1.88-1.78 (m, 2H).

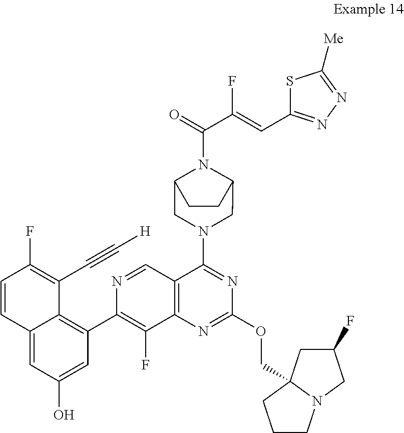 [SMILES: C#Cc1c(F)ccc2cc(O)cc(-c3ncc4c(N5CC6CCC(C5)N6C(=O)/C(F)=C/c5nnc(C)s5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(5-methyl-1,3,4-thiadiazol-2-yl)prop-2-en-1-one

4-(4-((1R, 5S)-3,8-Diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (5.0 mg, 0.008 mmol) and (Z)-2-fluoro-3-(5-methyl-1,3,4-thiadiazol-2-yl)acrylic acid (3.1 mg, 0.017 mmol) were combined as solids and dissolved in DMF (500 μL). 1-Methylimidazole (7 μL, 0.083 mmol) was added followed by chloro-N,N,N′,N′-tetramethylformamidinium hexafluorophosphate (4.7 mg, 0.017 mmol). The reaction mixture was stirred for 5 min. A saturated aqueous solution of ammonium hydroxide (100 μL) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 81% 5:95 MeCN:H₂O with 10 mM AA/19% 95:5 MeCN:H₂O with 10 mM AA→100% 95:5 MeCN:H₂O with 10 mM AA; λ=220 nm) to provide the desired product (3.0 mg, 3.9 μmol, 47% yield) as a brown solid. LC/MS (ESI) m/z: [M+H]⁺ calcd for C₃₉H₃₅F₄N8O₃S 771.2; found 771.0; ¹H NMR (500 MHz, DMSO-d₆) δ 10.17 (br s, 1H), 9.07 (s, 1H), 7.97 (dd, J=8.5, 6.1 Hz, 1H), 7.46 (t, J=8.9 Hz, 1H), 7.40 (d, J=2.4 Hz, 1H), 7.30 (d, J=35.5 Hz, 1H), 7.18 (s, 1H), 0.00 (d, J=54.3 Hz, 1H), 4.87-4.78 (m, 2H), 4.75-4.63 (m, 1H), 4.58-4.47 (m, 1H), 4.21-4.12 (m, 1H), 4.12-4.04 (m, 1H), 3.93 (s, 1H), 3.88-3.70 (m, 2H), 3.20-3.02 (m, 4H), 2.90-2.83 (m, 1H), 2.81 (s, 3H), 2.20-1.97 (m, 4H), 1.89-1.75 (m, 5H).

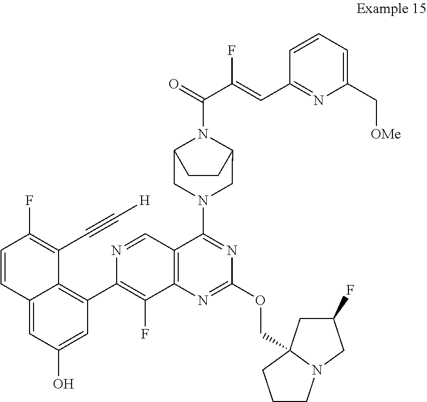 [SMILES: C#Cc1c(F)ccc2cc(O)cc(-c3ncc4c(N5CC6CCC(C5)N6C(=O)/C(F)=C/c5cccc(COC)n5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(6-(methoxymethyl)pyridin-2-yl)prop-2-en-1-one

4-(4-((1R, 5S)-3,8-Diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (5.0 mg, 0.008 mmol) and (Z)-2-fluoro-3-(6-(methoxymethyl)pyridin-2-yl)acrylic acid, sodium chloride adduct (5 mg, 0.017 mmol) were combined as solids and dissolved in DMF (500 μL). 1-Methylimidazole (7 μL, 0.083 mmol) was added followed by chloro-N,N,N′,N′-tetramethylformamidinium hexafluorophosphate (4.7 mg, 0.017 mmol). The reaction mixture was stirred for 5 min. A saturated aqueous solution of ammonium hydroxide (100 μL) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 78% 5:95 MeCN:H₂O with 10 mM AA/22% 95:5 MeCN:H₂O with 10 mM AA→100% 95:5 MeCN:H₂O with 10 mM AA; λ=220 nm) to provide the desired product (4.3 mg, 5.4 μmol, 65% yield) as a brown solid. LC/MS (ESI) m/z: [M+H]⁺ calcd for C₄₃H₃₉F₄N704 794.4; found 794.4; ¹H NMR (500 MHz, DMSO-d₆) δ 10.21 (br s, 1H), 9.07 (s, 1H), 7.97 (br dd, J=9.0, 6.3 Hz, 1H), 7.91 (t, J=7.9 Hz, 1H), 7.73 (d, J=7.7 Hz, 1H), 7.46 (t, J=9.0 Hz, 1H), 7.42-7.37 (m, 2H), 7.18 (s, 1H), 0.00 (d, J=37.8 Hz, 1H), 5.31 (d, J=53.7 Hz, 1H), 4.86-4.78 (m, 2H), 4.72-4.66 (m, 1H), 4.57-4.46 (m, 3H), 4.24-4.03 (m, 2H), 3.92 (br s, 1H), 3.87-3.71 (m, 2H), 3.38 (br s, 3H), 3.22-3.04 (m, 4H), 2.91-2.81 (m, 1H), 2.19-1.97 (m, 4H), 1.90-1.78 (m, 5H).

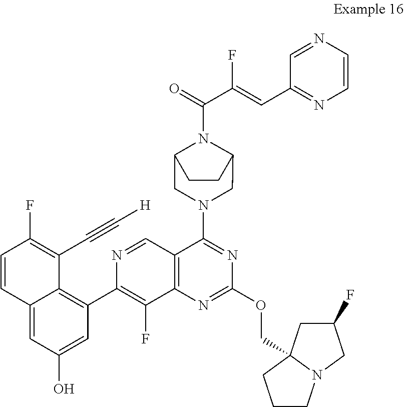 [SMILES: C#Cc1c(F)ccc2cc(O)cc(-c3ncc4c(N5CC6CCC(C5)N6C(=O)/C(F)=C/c5cnccn5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(pyrazin-2-yl)prop-2-en-1-one

4-(4-((1R, 5S)-3,8-Diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (25.0 mg, 0.042 mmol) and (Z)-2-fluoro-3-(pyrazin-2-yl)acrylic acid (21 mg, 0.125 mmol) were combined as solids and dissolved in DMF (400 L). 1-Methylimidazole (20 μL, 0.250 mmol) was added followed by chloro-N,N,N′,N′-tetramethylformamidinium hexafluorophosphate (35 mg, 0.125 mmol). The reaction mixture was stirred for 5 min. A saturated aqueous solution of ammonium hydroxide (100 L) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 81% 5:95 MeCN:H₂O with 10 mM AA/19% 95:5 MeCN:H₂O with 10 mM AA→100% 95:5 MeCN:H₂O with 10 mM AA; λ=220 nm) to provide the desired product (31.2 mg, 0.027 mmol, 65% yield) as a brown solid. LC/MS (ESI) m/z: [M+H]⁺ calcd for C₄₀H₃₅F₄N₈O_{3 751.3}; found 751.2; ¹H NMR (500 MHz, DMSO-d₆) δ 10.18 (br s, 1H), 9.08 (s, 1H), 8.99 (d, J=1.4 Hz, 1H), 8.75 (dd, J=2.4, 1.6 Hz, 1H), 8.62 (d, J=2.4 Hz, 1H), 7.97 (dd, J=9.2, 6.0 Hz, 1H), 7.46 (t, J=9.0 Hz, 1H), 7.39 (d, J=2.5 Hz, 1H), 7.18 (s, 1H), 6.90 (d, J=37.7 Hz, 1H),−0.01 (d, J=54.6 Hz, 1H), 4.89-4.80 (m, 2H), 4.73-4.66 (m, 1H), 4.58-4.47 (m, 1H), 4.21-4.13 (m, 1H), 4.12-4.03 (m, 1H), 3.93 (s, 1H), 3.88-3.68 (m, 2H), 3.20-3.02 (m, 4H), 2.90-2.81 (m, 1H), 2.19-1.98 (m, 4H), 1.90-1.77 (m, 5H).

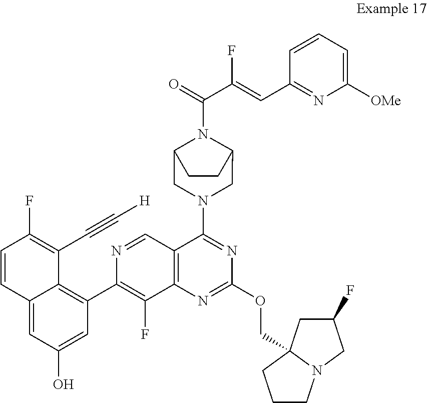 [SMILES: C#Cc1c(F)ccc2cc(O)cc(-c3ncc4c(N5CC6CCC(C5)N6C(=O)/C(F)=C/c5cccc(OC)n5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(6-methoxypyridin-2-yl)prop-2-en-1-one

4-(4-((1R, 5S)-3,8-Diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (5.0 mg, 0.008 mmol) and (Z)-2-fluoro-3-(6-methoxypyridin-2-yl)acrylic acid (3.1 mg, 0.017 mmol) were combined as solids and dissolved in DMF (500 μL). 1-Methylimidazole (7 μL, 0.083 mmol) was added followed by chloro-N,N,N′,N′-tetramethylformamidinium hexafluorophosphate (4.7 mg, 0.017 mmol). The reaction mixture was stirred for 5 min. A saturated aqueous solution of ammonium hydroxide (100 μL) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 70% 5:95 MeCN:H₂O with 10 mM AA/30% 95:5 MeCN:H₂O with 10 mM AA→100% 95:5 MeCN:H₂O with 10 mM AA; λ=220 nm) to provide the desired product (4.3 mg, 5.5 μmol, 66% yield) as a brown solid. LC/MS (ESI) m/z: [M+H]⁺ calcd for C₄₂H₃₈F₄N₇O₄ 780.3; found 780.3; ¹H NMR (500 MHz, DMSO-d₆) δ 10.22 (br s, 1H), 9.06 (s, 1H), 8.00-7.92 (m, 1H), 7.79 (t, J=7.6 Hz, 1H), 7.45 (t, J=9.0 Hz, 1H), 7.42-7.37 (m, 2H), 7.18 (s, 1H), 6.82 (d, J=8.4 Hz, 1H), 6.74 (d, J=37.3 Hz, 1H), 5.28 (d, J=54.2 Hz, 1H), 4.81 (br s, 2H), 4.72-4.65 (m, 1H), 4.56-4.48 (m, 1H), 4.15 (d, J=10.6 Hz, 1H), 4.05 (br d, J=10.6 Hz, 1H), 3.89 (s, 1H), 3.87 (s, 3H), 3.84-3.70 (m, 2H), 3.20-3.01 (m, 4H), 2.88-2.78 (m, 1H), 2.18-1.97 (m, 4H), 1.89-1.74 (m, 5H).

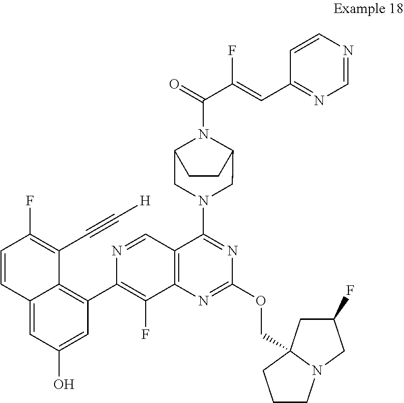 [SMILES: C#Cc1c(F)ccc2cc(O)cc(-c3ncc4c(N5CC6CCC(C5)N6C(=O)/C(F)=C/c5ccncn5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(pyrimidin-4-yl)prop-2-en-1-one

4-(4-((1R, 5S)-3,8-Diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (5.0 mg, 0.008 mmol) and (Z)-2-fluoro-3-(pyrimidin-4-yl)acrylic acid (7.0 mg, 0.042 mmol) were combined as solids and dissolved in DMF (500 L). 1-Methylimidazole (2 μL, 0.025 mmol) was added followed by chloro-N,N,N′,N′-tetramethylformamidinium hexafluorophosphate (4.7 mg, 0.017 mmol). The reaction mixture was stirred for 5 min. A saturated aqueous solution of ammonium hydroxide (100 L) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 83% 5:95 MeCN:H₂O with 10 mM AA/17% 95:5 MeCN:H₂O with 10 mM AA→100% 95:5 MeCN:H₂O with 10 mM AA; λ=220 nm) to provide the desired product (0.7 mg, 0.9 μmol, 11% yield) as a brown solid. LC/MS (ESI) m/z: [M+H]⁺ calcd for C₄₀H₃₅F₄N₈O₃ 751.3; found 751.2.

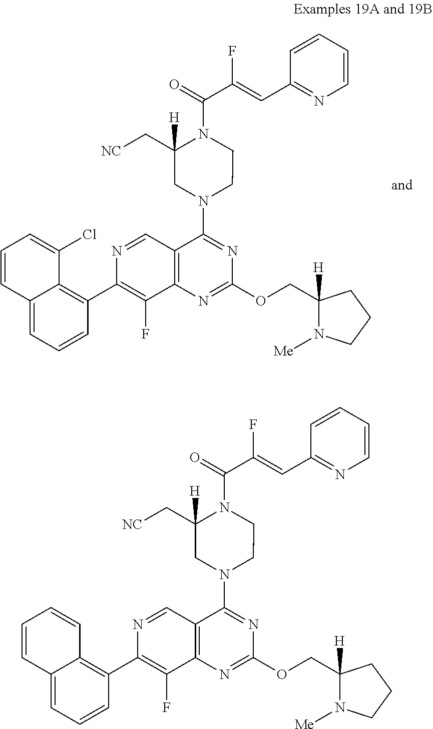 [SMILES: CN1CCC[C@H]1COc1nc(N2CCN(C(=O)/C(F)=C/c3ccccn3)[C@@H](CC#N)C2)c2cnc(-c3cccc4cccc(Cl)c34)c(F)c2n1 ; CN1CCC[C@H]1COc1nc(N2CCN(C(=O)/C(F)=C/c3ccccn3)[C@@H](CC#N)C2)c2cnc(-c3cccc4ccccc34)c(F)c2n1]

### 2-((S)-4-(7-(8-chloronaphthalen-1-yl)-8-fluoro-2-(((S)-1-methylpyrrolidin-2-yl)methoxy)pyrido[4,3-d]pyrimidin-4-yl)-1-((Z)-2-fluoro-3-(pyridin-2-yl)acryloyl)piperazin-2-yl)acetonitrile and 2-((S)-4-(8-fluoro-2-(((S)-1-methylpyrrolidin-2-yl)methoxy)-7-(naphthalen-1-yl)pyrido[4,3-d]pyrimidin-4-yl)-1-((Z)-2-fluoro-3-(pyridin-2-yl)acryloyl)piperazin-2-yl)acetonitrile

2-((S)-4-(7-(8-Chloronaphthalen-1-yl)-8-fluoro-2-(((S)-1-methylpyrrolidin-2-yl)methoxy)pyrido[4,3-d]pyrimidin-4-yl)piperazin-2-yl)acetonitrile, containing an unquantified amount of 2-((S)-4-(8-fluoro-2-(((S)-1-methylpyrrolidin-2-yl)methoxy)-7-(naphthalen-1-yl)pyrido[4,3-d]pyrimidin-4-yl)piperazin-2-yl)acetonitrile, (65 mg, 0.12 mmol) and (Z)-2-fluoro-3-(pyridin-2-yl)acrylic acid (60 mg, 0.36 mmol) were combined as solids and dissolved in DMF (1.2 mL). 1-Methylimidazole (57 μL, 0.71 mmol) was added followed by chloro-N,N,N′,N′-tetramethylformamidinium hexafluorophosphate (100 mg, 0.36 mmol). The reaction mixture was stirred for 5 min, and was directly purified by reverse phase HPLC (column: Xbridge C18, 19 mm×200 mm, 5 μm particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) followed by preparative SFC (column: Chiralpak IH, 21 mm×250 mm, 5 μm particles; flow rate: 85 mL/min; column temperature: 40° C.; isocratic: 70% CO₂:30% iPrOH with 0.1% NH₄₀H) to provide both title compounds. Compound 19A: 24 mg, 0.03 mmol, 29% yield; LC/MS (ESI) m/z: [M+H]⁺ calcd for C₃₇H₃₄C₁F₂N₈O₂ 695.2; found 695.2; ¹H NMR (600 MHz, CDCl₃) δ 9.26-9.07 (m, 1H), 8.68 (br d, J=4.4 Hz, 1H), 8.05-7.38 (m, 9H), 7.03-6.54 (m, 2H), 5.06-4.85 (m, 1H), 4.80-4.68 (m, 1H), 4.60-3.55 (m, 5H), 3.37-3.24 (m, 1H), 3.15-2.87 (m, 3H), 2.62 (br s, 3H), 2.51-2.40 (m, 1H), 2.20 -2.08 (m, 1H), 1.98-1.76 (m, 4H). Compound 19B: 13.6 mg, 0.02 mmol, 17% yield. LC/MS (ESI) m/z: [M+H]⁺ calcd for C₃₇H₃₅F₂N₈O₂ 661.3; found 661.3.

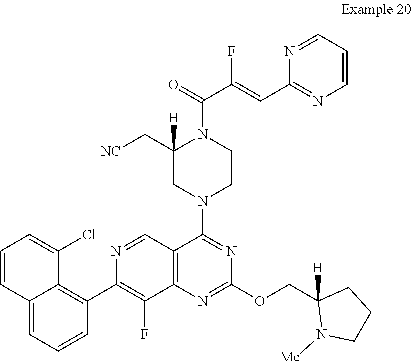 [SMILES: CN1CCC[C@H]1COc1nc(N2CCN(C(=O)/C(F)=C/c3ncccn3)[C@@H](CC#N)C2)c2cnc(-c3cccc4cccc(Cl)c34)c(F)c2n1]

### 2-((S)-4-(7-(8-chloronaphthalen-1-yl)-8-fluoro-2-(((S)-1-methylpyrrolidin-2-yl)methoxy)pyrido[4,3-d]pyrimidin-4-yl)-1-((Z)-2-fluoro-3-(pyrimidin-2-yl)acryloyl)piperazin-2-yl)acetonitrile

2-((S)-4-(7-(8-Chloronaphthalen-1-yl)-8-fluoro-2-(((S)-1-methylpyrrolidin-2-yl)methoxy)pyrido[4,3-d]pyrimidin-4-yl)piperazin-2-yl)acetonitrile (7 mg, 0.01 mmol) and (Z)-2-fluoro-3-(pyrimidin-2-yl)acrylic acid (3.4 mg, 0.02 mmol) were combined as solids and dissolved in DMF (0.5 mL). 1-Methylimidazole (9 μL, 0.11 mmol) was added followed by chloro-N,N,N′,N′-tetramethylformamidinium hexafluorophosphate (6.1 mg, 0.02 mmol). The reaction mixture was stirred for 5 min, and was directly purified by reverse phase HPLC (column: Xbridge C18, 19 mm×200 mm, 5 μm particles; flow rate: 20 mL/min; column temperature: 25° C.; gradient: 84% 5:95 MeCN:H₂O with 10 mM AA/16% 95:5 MeCN:H₂O with 10 mM AA→100% 95:5 MeCN:H₂O with 10 mM AA; λ=220 nm) to provide the desired product (4.9 mg, 0.007 mmol, 64% yield). LC/MS (ESI) m/z: [M+H]⁺ calcd for C₃₆H₃₃C₁F₂N₉O₂ 696.2; found 696.2; ¹H NMR (500 MHz, DMSO-d₆) δ 9.31-9.15 (m, 1H), 8.93-8.84 (m, 2H), 8.16 (br d, J=15.2 Hz, 2H), 7.78-7.41 (m, 6H), 6.63 (d, J=35.1 Hz, 1H), 4.96-4.73 (m, 1H), 4.60-4.36 (m, 3H), 4.30-4.10 (m, 3H), 4.01-3.93 (m, 1H), 3.87-3.76 (m, 1H), 3.21-3.09 (m, 1H), 2.98-2.89 (m, 1H), 2.64-2.55 (m, 1H), 2.39-2.33 (m, 3H), 2.22-2.13 (m, 1H), 1.99-1.90 (m, 1H), 1.73-1.58 (m, 3H).

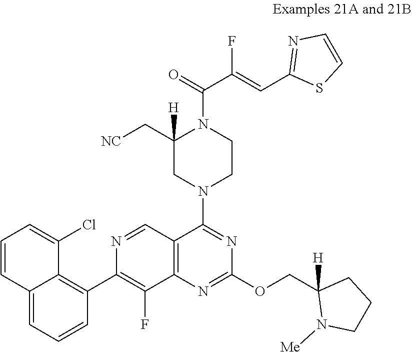 [SMILES: CN1CCC[C@H]1COc1nc(N2CCN(C(=O)/C(F)=C/c3nccs3)[C@@H](CC#N)C2)c2cnc(-c3cccc4cccc(Cl)c34)c(F)c2n1]

### 2-((S)-4-(7-(8-chloronaphthalen-1-yl)-8-fluoro-2-(((S)-1-methylpyrrolidin-2-yl)methoxy)pyrido[4,3-d]pyrimidin-4-yl)-1-((Z)-2-fluoro-3-(thiazol-2-yl)acryloyl)piperazin-2-yl)acetonitrile and

### 2-((S)-4-(8-fluoro-2-(((S)-1-methylpyrrolidin-2-yl)methoxy)-7-(naphthalen-1-yl)pyrido[4,3-d]pyrimidin-4-yl)-1-((Z)-2-fluoro-3-(pyridin-2-yl)acryloyl)piperazin-2-yl)acetonitrile

2-((S)-4-(7-(8-Chloronaphthalen-1-yl)-8-fluoro-2-(((S)-1-methylpyrrolidin-2-yl)methoxy)pyrido[4,3-d]pyrimidin-4-yl)piperazin-2-yl)acetonitrile, containing an unquantified amount of 2-((S)-4-(8-fluoro-2-(((S)-1-methylpyrrolidin-2-yl)methoxy)-7-(naphthalen-1-yl)pyrido[4,3-d]pyrimidin-4-yl)piperazin-2-yl)acetonitrile, (35 mg, 0.06 mmol) and (Z)-2-fluoro-3-(thiazol-2-yl)acrylic acid (33 mg, 0.19 mmol) were combined as solids and dissolved in DMF (0.6 mL). 1-Methylimidazole (31 μL, 0.39 mmol) was added followed by chloro-N,N,N′,N′-tetramethylformamidinium hexafluorophosphate (54 mg, 0.19 mmol). The reaction mixture was stirred for 5 min, and was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) followed by preparative SFC (column: Chiralpak IH, 21 mm×250 mm, 5 m particles; flow rate: 85 mL/min; column temperature: 40° C.; isocratic: 70% CO₂:30% iPrOH with 0.1% NH₄₀H) to provide both title compounds. Compound 21A: 7.2 mg, 0.01 mmol, 16% yield; LC/MS (ESI) m/z: [M+H]⁺ calcd for C₃₅H₃₂C₁F₂N₈O₂ S 701.2; found 701.2; ¹H NMR (500 MHz, DMSO-d₆) δ 9.31-9.12 (m, 1H), 8.16 (s, 4H), 7.77-7.48 (m, 5H), 7.10 (d, J=38.9 Hz, 1H), 4.97-4.84 (m, 1H), 4.57-4.39 (m, 3H), 4.31-4.10 (m, 3H), 3.87-3.77 (m, 2H), 3.14-3.07 (m, 1H), 2.98-2.89 (m, 1H), 2.63-2.56 (m, 1H), 2.41-2.32 (m, 3H), 2.22-2.13 (m, 1H), 2.04-1.90 (m, 1H), 1.72-1.62 (m, 3H). Compound 21B: 3.1 mg, 0.005 mmol, 7% yield; LC/MS (ESI) m/z: [M+H]⁺ calcd for C₃₅H₃₃F₂N₈O₂ S 667.2; found 667.2; ¹H NMR (500 MHz, DMSO-d₆) δ 9.30 (s, 1H), 8.13-7.99 (m, 4H), 7.72-7.66 (m, 3H), 7.62-7.56 (m, 1H), 7.55-7.48 (m, 1H), 0.00 (d, J=38.3 Hz, 1H), 4.97-4.86 (m, 1H), 4.60-4.34 (m, 4H), 4.26-4.06 (m, 1H), 3.87-3.78 (m, 1H), 3.18-3.07 (m, 2H), 2.10-1.96 (m, 1H), 1.85-1.63 (m, 3H).

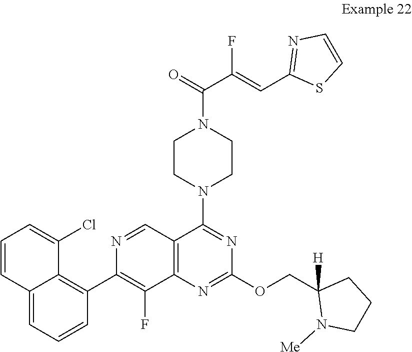 [SMILES: CN1CCC[C@H]1COc1nc(N2CCN(C(=O)/C(F)=C/c3nccs3)CC2)c2cnc(-c3cccc4cccc(Cl)c34)c(F)c2n1]

### (S,Z)-1-(4-(7-(8-chloronaphthalen-1-yl)-8-fluoro-2-((1-methylpyrrolidin-2-yl)methoxy)pyrido[4,3-d]pyrimidin-4-yl)piperazin-1-yl)-2-fluoro-3-(thiazol-2-yl)prop-2-en-1-one

(S)-7-(8-Chloronaphthalen-1-yl)-8-fluoro-2-((1-methylpyrrolidin-2-yl)methoxy)-4-(piperazin-1-yl)pyrido[4,3-d]pyrimidine (5 mg, 0.01 mmol) and (Z)-2-fluoro-3-(thiazol-2-yl)acrylic acid (3.4 mg, 0.02 mmol) were combined as solids and dissolved in DMF (0.5 mL). 1-Methylimidazole (8 μL, 0.10 mmol) was added followed by chloro-N,N,N′,N′-tetramethylformamidinium hexafluorophosphate (5.5 mg, 0.02 mmol). The reaction mixture was stirred for 5 min, and was directly purified by reverse phase HPLC (column: Xbridge C18, 19 mm×200 mm, 5 μm particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (5.3 mg, 0.008 mmol, 80% yield). LC/MS (ESI) m/z: [M+H]⁺ calcd for C₃₃H₃₁C₁F₂N₇O₂ S 662.2; found 662.2; ¹H NMR (500 MHz, DMSO-d₆) δ 9.30-9.12 (m, 1H), 8.26-7.96 (m, 4H), 7.80-7.47 (m, 4H), 7.10 (d, J=37.8 Hz, 1H), 4.49-4.36 (m, 1H), 4.29-4.21 (m, 1H), 4.19-4.12 (m, 4H), 4.03-3.86 (m, 4H), 2.99-2.90 (m, 1H), 2.64-2.57 (m, 1H), 2.39-2.33 (m, 3H), 2.22-2.14 (m, 1H), 1.99-1.92 (m, 1H), 1.73-1.58 (m, 3H).

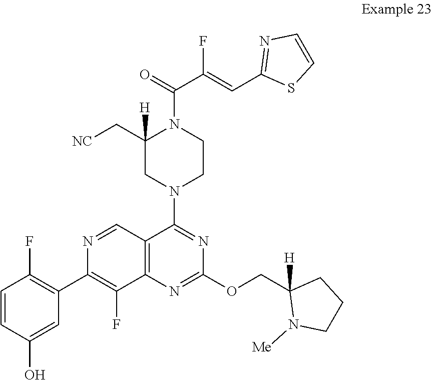 [SMILES: CN1CCC[C@H]1COc1nc(N2CCN(C(=O)/C(F)=C/c3nccs3)[C@@H](CC#N)C2)c2cnc(-c3cc(O)ccc3F)c(F)c2n1]

### 2-((S)-1-((Z)-2-fluoro-3-(thiazol-2-yl)acryloyl)-4-(8-fluoro-7-(2-fluoro-5-hydroxyphenyl)-2-(((S)-1-methylpyrrolidin-2-yl)methoxy)pyrido[4,3-d]pyrimidin-4-yl)piperazin-2-yl)acetonitrile

(2-((S)-4-(8-Fluoro-7-(2-fluoro-5-hydroxyphenyl)-2-(((S)-1-methylpyrrolidin-2-yl)methoxy)pyrido[4,3-d]pyrimidin-4-yl)piperazin-2-yl)acetonitrile (24 mg, 0.048 mmol) and (Z)-2-fluoro-3-(thiazol-2-yl)acrylic acid (25 mg, 0.15 mmol) were combined as solids and dissolved in DMF (1.0 mL). 1-Methylimidazole (23 μL, 0.29 mmol) was added followed by chloro-N,N,N′,N′-tetramethylformamidinium hexafluorophosphate (41 mg, 0.15 mmol). The reaction mixture was stirred for 5 min. Sodium hydroxide solution (1.0 M, 0.2 mL) was added, and the mixture was stirred for an additional 5 min. The solution was directly purified by reverse phase HPLC (column: Xbridge C18, 19 mm×200 mm, 5 m particles; flow rate: 20 mL/min; column temperature: 25° C.; gradient: 91% 5:95 MeCN:H₂O with 10 mM AA/9% 95:5 MeCN:H₂O with 10 mM AA→100% 95:5 MeCN:H₂O with 10 mM AA; λ=220 nm) to provide the desired product (12.3 mg, 0.018 mmol, 38% yield). LC/MS (ESI) m/z: [M+H]⁺ calcd for C₃₁H₃₀F₃NsO₃S 651.2; found 651.3; ¹H NMR (500 MHz, DMSO-d₆) δ 9.68 (br s, 1H), 9.23 (s, 1H), 8.05-7.98 (m, 2H), 7.18 (t, J=9.3 Hz, 1H), 0.00 (d, J=37.8 Hz, 1H), 7.04-7.00 (m, 1H), 6.94-6.90 (m, 1H), 4.98-4.81 (m, 1H), 4.58-4.40 (m, 3H), 4.32-4.22 (m, 1H), 4.16-4.04 (m, 2H), 3.87-3.72 (m, 2H), 3.12-3.04 (m, 2H), 3.01-2.94 (m, 1H), 2.67-2.57 (m, 1H), 2.39 (s, 3H), 2.27-2.16 (m, 1H), 2.01-1.93 (m, 1H), 1.74-1.61 (m, 3H).

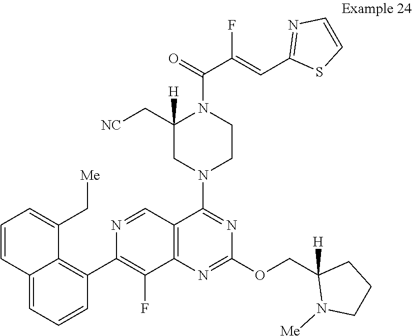 [SMILES: CCc1cccc2cccc(-c3ncc4c(N5CCN(C(=O)/C(F)=C/c6nccs6)[C@@H](CC#N)C5)nc(OC[C@@H]5CCCN5C)nc4c3F)c12]

### 2-((S)-4-(7-(8-ethylnaphthalen-1-yl)-8-fluoro-2-(((S)-1-methylpyrrolidin-2-yl)methoxy)pyrido[4,3-d]pyrimidin-4-yl)-1-((Z)-2-fluoro-3-(thiazol-2-yl)acryloyl)piperazin-2-yl)acetonitrile

2-((S)-4-(7-(8-Ethylnaphthalen-1-yl)-8-fluoro-2-(((S)-1-methylpyrrolidin-2-yl)methoxy)pyrido[4,3-d]pyrimidin-4-yl)piperazin-2-yl)acetonitrile (24 mg, 0.044 mmol) and (Z)-2-fluoro-3-(thiazol-2-yl)acrylic acid (21 mg, 0.13 mmol) were combined as solids and dissolved in DMF (0.9 mL). 1-Methylimidazole (23 μL, 0.27 mmol) was added followed by chloro-N,N,N′,N′-tetramethylformamidinium hexafluorophosphate (37 mg, 0.13 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (3.0 mg, 0.004 mmol, 7% yield over 3 steps). LC/MS (ESI) m/z: [M+H]⁺ calcd for C₃₇H₃₇F₂N₈O₂ S 695.3; found 695.5; ¹H NMR (500 MHz, DMSO-d₆) δ 9.20 (s, 1H), 8.13-7.90 (m, 3H), 7.63-7.37 (m, 5H), 7.10 (d, J=37.6 Hz, 1H), 4.95-4.85 (m, 1H), 4.60-4.28 (m, 4H), 4.11 (br s, 1H), 3.15-3.07 (m, 2H), 3.06-2.96 (m, 1H), 2.79-2.68 (m, 1H), 2.42 (br s, 3H), 2.38-2.20 (m, 3H), 2.03-1.93 (m, 1H), 1.75-1.62 (m, 3H), 1.61-1.50 (m, 1H), 0.93 (t, J=7.4 Hz, 2H), 0.85 (q, J=7.4 Hz, 3H).

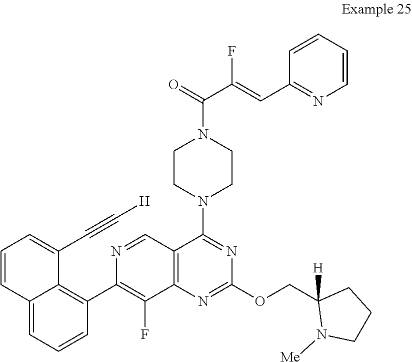 [SMILES: C#Cc1cccc2cccc(-c3ncc4c(N5CCN(C(=O)/C(F)=C/c6ccccn6)CC5)nc(OC[C@@H]5CCCN5C)nc4c3F)c12]

### (S,Z)-1-(4-(7-(8-ethynylnaphthalen-1-yl)-8-fluoro-2-((1-methylpyrrolidin-2-yl)methoxy)pyrido[4,3-d]pyrimidin-4-yl)piperazin-1-yl)-2-fluoro-3-(pyridin-2-yl)prop-2-en-1-one

(S)-7-(8-Ethynylnaphthalen-1-yl)-8-fluoro-2-((1-methylpyrrolidin-2-yl)methoxy)-4-(piperazin-1-yl)pyrido[4,3-d]pyrimidine (6 mg, 0.012 mmol) and (Z)-2-fluoro-3-(pyridin-2-yl)acrylic acid (6 mg, 0.04 mmol) were combined as solids and dissolved in DMF (0.3 mL). 1-Methylimidazole (6 μL, 0.072 mmol) was added followed by chloro-N,N,N′,N′-tetramethylformamidinium hexafluorophosphate (10 mg, 0.036 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (3.1 mg, 0.004, 40% yield). LC/MS (ESI) m/z: [M+H]⁺ calcd for C₃₇H₃₄F₂N₇O₂; found 695.5; ¹H NMR (500 MHz, DMSO-d₆) δ 9.20 (s, 1H), 8.13-7.90 (m, 3H), 7.63-7.37 (m, 5H), 7.10 (d, J=37.6 Hz, 1H), 4.95-4.85 (m, 1H), 4.60-4.28 (m, 4H), 4.11 (br s, 1H), 3.15-3.07 (m, 2H), 3.06-2.96 (m, 1H), 2.79-2.68 (m, 1H), 2.42 (br s, 3H), 2.38-2.20 (m, 3H), 2.03-1.93 (m, 1H), 1.75-1.62 (m, 3H), 1.61-1.50 (m, 1H), 0.93 (t, J=7.4 Hz, 2H), 0.85 (q, J=7.4 Hz, 3H).

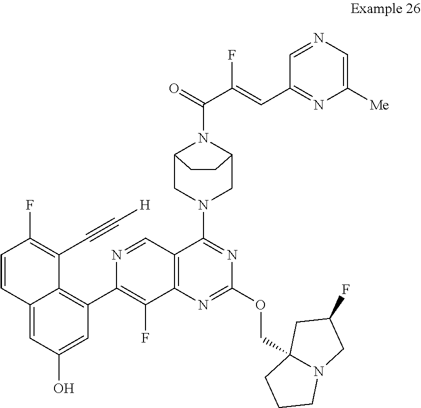 [SMILES: C#Cc1c(F)ccc2cc(O)cc(-c3ncc4c(N5CC6CCC(C5)N6C(=O)/C(F)=C/c5cncc(C)n5)nc(OC[C@@]56CCCN5C[C@H](F)C6)nc4c3F)c12]

### (Z)-1-((1R, 5S)-3-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octan-8-yl)-2-fluoro-3-(6-methylpyrazin-2-yl)prop-2-en-1-one

4-(4-((1R, 5S)-3,8-Diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)-5-ethynyl-6-fluoronaphthalen-2-ol (10 mg, 0.017 mmol) and (Z)-2-fluoro-3-(6-methylpyrazin-2-yl)acrylic acid (6 mg, 0.033 mmol) were combined as solids and dissolved in DMF (500 μL). 1-Methylimidazole (13 μL, 0.17 mmol) was added followed by chloro-N,N,N′,N′-tetramethylformamidinium hexafluorophosphate (9.3 mg, 0.03 mmol). The reaction mixture was stirred for 5 min. A saturated aqueous solution of ammonium hydroxide (100 μL) was added, and the reaction mixture was stirred for an additional 5 min. The reaction mixture was directly purified by reverse phase HPLC (column: Xbridge C_{18, 19} mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (11.3 mg, 0.014 mmol, 89% yield) as a brown solid. LC/MS (ESI) m/z: [M+H]⁺ calcd for C₄₁H₃₆F₄N₈O₃ 765.3; found 765.2.

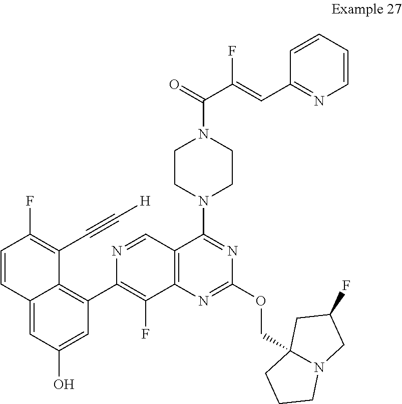 [SMILES: C#Cc1c(F)ccc2cc(O)cc(-c3ncc4c(N5CCN(C(=O)/C(F)=C/c6ccccn6)CC5)nc(OC[C@@]56CCCN5C[C@H](F)C6)nc4c3F)c12]

### (Z)-1-(4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridin-2-yl)prop-2-en-1-one

5-Ethynyl-6-fluoro-4-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol (20 mg, 0.035 mmol) and (Z)-2-fluoro-3-(pyridin-2-yl)acrylic acid (17 mg, 0.10 mmol) were combined as solids and dissolved in DMF (350 μL). 1-Methylimidazole (17 μL, 0.17 mmol) was added followed by chloro-N,N,N′,N′-tetramethylformamidinium hexafluorophosphate (29 mg, 0.10 mmol). The reaction mixture was stirred for 5 min. A saturated aqueous solution of ammonium hydroxide (100 μL) was added, and the reaction mixture was stirred for an additional 5 min. The reaction mixture was directly purified by reverse phase HPLC (column: Xbridge C18, 19 mm×200 mm, 5 μm particles; flow rate: 20 mL/min; column temperature: 25° C.; gradient: 81% 5:95 MeCN:H₂O with 10 mM AA/19% 95:5 MeCN:H₂O with 10 mM AA→100% 95:5 MeCN:H₂O with 10 mM AA; λ=220 nm) to provide the desired product (15.6 mg, 0.021 mmol, 61% yield) as a brown solid. LC/MS (ESI) m/z: [M+H]⁺ calcd for C₃₉H₃₄F₄N₇O₃ 724.3; found 724.2; ¹H NMR (500 MHz, DMSO-d₆) δ 10.17 (s, 1H), 9.11 (s, 1H), 8.65 (dd, J=4.4, 1.3 Hz, 1H), 7.97 (dd, J=9.3, 6.0 Hz, 1H), 7.91-7.86 (m, 1H), 7.79 (d, J=7.9 Hz, 1H), 7.46 (t, J=9.0 Hz, 1H), 7.41-7.35 (m, 2H), 7.19 (s, 1H), 6.66 (d, J=38.7 Hz, 1H), 5.28 (d, J=54.2 Hz, 1H), 4.18-4.03 (m, 7H), 3.97 (s, 1H), 3.95-3.80 (m, 2H), 3.16-3.00 (m, 4H), 2.88-2.78 (m, 1H), 2.15-2.10 (m, 1H), 2.08-2.06 (m, 1H), 2.05-1.97 (m, 1H), 1.88-1.73 (m, 3H).

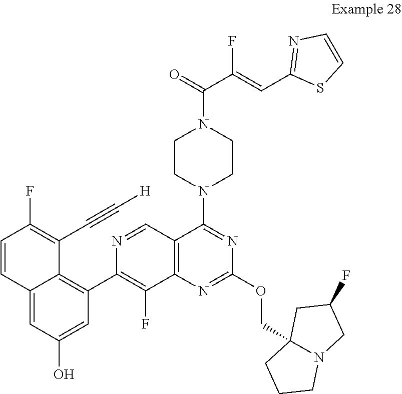 [SMILES: C#Cc1c(F)ccc2cc(O)cc(-c3ncc4c(N5CCN(C(=O)/C(F)=C/c6nccs6)CC5)nc(OC[C@@]56CCCN5C[C@H](F)C6)nc4c3F)c12]

### (Z)-1-(4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(thiazol-2-yl)prop-2-en-1-one

5-Ethynyl-6-fluoro-4-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol (20 mg, 0.035 mmol) and (Z)-2-fluoro-3-(thiazol-2-yl)acrylic acid (18 mg, 0.10 mmol) were combined as solids and dissolved in DMF (350 μL). 1-methylimidazole (17 μL, 0.17 mmol) was added followed by chloro-N,N,N′,N′-tetramethylformamidinium hexafluorophosphate (29 mg, 0.10 mmol). The reaction mixture was stirred for 5 min. A saturated aqueous solution of ammonium hydroxide (100 μL) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (11 mg, 0.015 mmol, 43% yield) as a brown solid. LC/MS (ESI) m/z: [M+H]⁺ calcd for C₃₇H₃₂F₄N₇O₃S 730.2; found 730.2; ¹H NMR (500 MHz, DMSO-d₆) δ 10.17 (s, 1H), 9.11 (s, 1H), 8.06-7.94 (m, 3H), 7.46 (t, J=9.0 Hz, 1H), 7.40 (d, J=2.5 Hz, 1H), 7.20 (s, 1H), 7.11 (d, J=37.5 Hz, 1H), 0.00 (d, J=54.4 Hz, 1H), 4.22-4.04 (m, 7H), 3.97 (s, 1H), 3.96-3.86 (m, 4H), 3.16-3.02 (m, 2H), 2.95-2.79 (m, 1H), 2.22-2.08 (m, 2H), 2.06-1.99 (m, 1H), 1.90-1.74 (m, 3H).

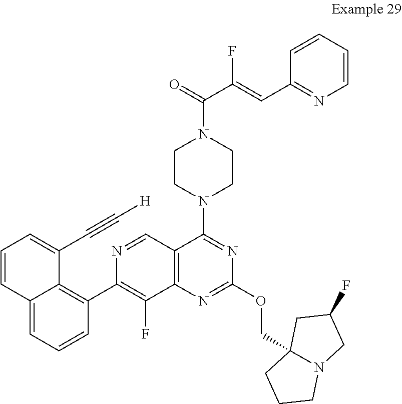 [SMILES: C#Cc1cccc2cccc(-c3ncc4c(N5CCN(C(=O)/C(F)=C/c6ccccn6)CC5)nc(OC[C@@]56CCCN5C[C@H](F)C6)nc4c3F)c12]

### (Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (33 mg, 0.06 mmol), and (Z)-2-fluoro-3-(pyridin-2-yl)acrylic acid (20 mg, 0.12 mmol) were combined as solids and dissolved in DMF (610 μL). 1-Methylimidazole (29 μL, 0.37 mmol) was added followed by chloro-N,N,N′,N′-tetramethylformamidinium hexafluorophosphate (34 mg, 0.12 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 72% 5:95 MeCN:H₂O with 10 mM AA/28% 95:5 MeCN:H₂O with 10 mM AA→100% 95:5 MeCN:H₂O with 10 mM AA; λ=220 nm) to provide the desired product (23 mg, 0.033 mmol, 53% yield) as a brown solid. LC/MS (ESI) m/z: [M+H]⁺ calcd for C₃₉H₃₅F₃N₇O₂ 690.3; found 690.2; ¹H NMR (500 MHz, DMSO-d₆) δ 9.12 (s, 1H), 8.68-8.61 (m, 1H), 8.19-8.11 (m, 2H), 7.93-7.84 (m, 1H), 7.79 (d, J=7.9 Hz, 1H), 7.73-7.68 (m, 2H), 7.63-7.55 (m, 2H), 7.41-7.31 (m, 1H), 6.66 (d, J=38.5 Hz, 1H), 5.28 (d, J=54.4 Hz, 1H), 4.19-4.08 (m, 5H), 4.00-3.82 (m, 4H), 3.71 (s, 1H), 3.13-3.01 (m, 4H), 2.86-2.78 (m, 1H), 2.15-2.11 (m, 1H), 2.07-2.04 (m, 1H), 2.03-1.99 (m, 1H), 1.89-1.75 (m, 3H).

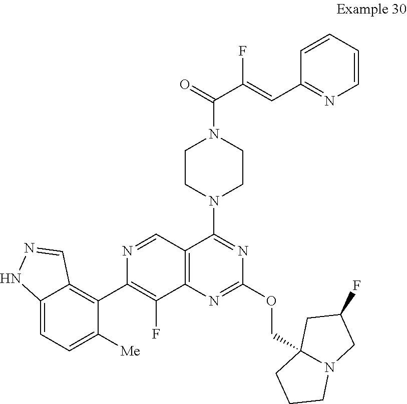 [SMILES: Cc1ccc2[nH]ncc2c1-c1ncc2c(N3CCN(C(=O)/C(F)=C/c4ccccn4)CC3)nc(OC[C@@]34CCCN3C[C@H](F)C4)nc2c1F]

### (Z)-2-fluoro-1-(4-(8-fluoro-2-(((2R, 7aS)-2-fluorotetrahydro-1H-pyrrolizin-7a(5H)-yl)methoxy)-7-(5-methyl-1H-indazol-4-yl)pyrido[4,3-d]pyrimidin-4-yl)piperazin-1-yl)-3-(pyridin-2-yl)prop-2-en-1-one

8-Fluoro-2-(((2R, 7aS)-2-fluorotetrahydro-1H-pyrrolizin-7a(5H)-yl)methoxy)-7-(5-methyl-1H-indazol-4-yl)-4-(piperazin-1-yl)pyrido[4,3-d]pyrimidine, HCl salt (7 mg, 0.013 mmol) and (Z)-2-fluoro-3-(pyridin-2-yl)acrylic acid (4.5 mg, 0.027 mmol) were combined as solids and dissolved in DMF (500 μL). 1-methylimidazole (11 μL, 0.13 mmol) was added followed by chloro-N,N,N′,N′-tetramethylformamidinium hexafluorophosphate (7.6 mg, 0.27 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 86% 5:95 MeCN:H₂O with 10 mM AA/14% 95:5 MeCN:H₂O with 10 mM AA→100% 95:5 MeCN:H₂O with 10 mM AA; λ=220 nm) to provide the desired product (5.9 mg, 0.009 mmol, 65% yield) as a brown solid.LC/MS (ESI) m/z: [M+H]⁺ calcd for C₃₅H₃₅F₃N₉O₂ 670.3; found 670.2; ¹H NMR (500 MHz, DMSO-d₆) δ 13.12 (br s, 1H), 9.27 (s, 1H), 8.67-8.59 (m, 1H), 7.94-7.85 (m, 1H), 7.79 (d, J=8.1 Hz, 1H), 7.66 (s, 1H), 7.59 (d, J=8.5 Hz, 1H), 7.41-7.34 (m, 2H), 6.66 (d, J=38.8 Hz, 1H), 5.33 (d, J=54.9 Hz, 1H), 4.34-4.10 (m, 7H), 4.03-3.91 (m, 2H), 3.89-3.81 (m, 2H), 3.28-3.17 (m, 3H), 3.01-2.83 (m, 1H), 2.28 (s, 3H), 2.25-2.08 (m, 2H), 1.96-1.92 (m, 1H), 1.88-1.78 (m, 2H).

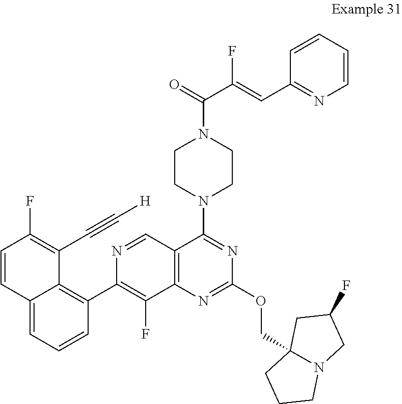 [SMILES: C#Cc1c(F)ccc2cccc(-c3ncc4c(N5CCN(C(=O)/C(F)=C/c6ccccn6)CC5)nc(OC[C@@]56CCCN5C[C@H](F)C6)nc4c3F)c12]

### (Z)-1-(4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridin-2-yl)prop-2-en-1-one

7-(8-Ethynyl-7-fluoronaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (18 mg, 0.032 mmol) and (Z)-2-fluoro-3-(pyridin-2-yl)acrylic acid (22 mg, 0.13 mmol) were combined as solids and dissolved in DMF (500 μL). 1-Methylimidazole (26 μL, 0.32 mmol) was added followed by chloro-N,N,N′,N′-tetramethylformamidinium hexafluorophosphate (36 mg, 0.13 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide the desired product (16.1 mg, 0.022 mmol, 70% yield) as a brown solid. LC/MS (ESI) m/z: [M+H]⁺ calcd for C₃₉H₃₄F₄N₇O₂ 708.3; found 708.3; ¹H NMR (500 MHz, DMSO-d₆) δ 9.14 (s, 1H), 8.65 (d, J=4.7 Hz, 1H), 8.26-8.18 (m, 2H), 7.93-7.85 (m, 1H), 7.82-7.76 (m, 1H), 7.73-7.65 (m, 2H), 7.63-7.58 (m, 1H), 7.44-7.35 (m, 1H), 6.66 (d, J=38.5 Hz, 1H), 5.33 (d, J=54.5 Hz, 1H), 4.27-4.07 (m, 8H), 4.05 (s, 1H), 4.02-3.89 (m, 3H), 3.24-3.17 (m, 2H), 3.00-2.86 (m, 1H), 2.29-2.03 (m, 3H), 1.91-1.75 (m, 3H).

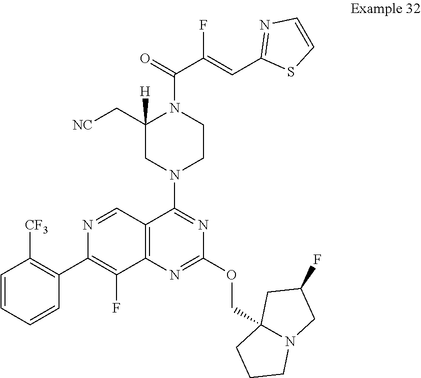 [SMILES: N#CC[C@H]1CN(c2nc(OC[C@@]34CCCN3C[C@H](F)C4)nc3c(F)c(-c4ccccc4C(F)(F)F)ncc23)CCN1C(=O)/C(F)=C/c1nccs1]

### 2-((S)-4-(8-fluoro-2-(((2R, 7aS)-2-fluorotetrahydro-1H-pyrrolizin-7a(5H)-yl)methoxy)-7-(2-(trifluoromethyl)phenyl)pyrido[4,3-d]pyrimidin-4-yl)-1-((Z)-2-fluoro-3-(thiazol-2-yl)acryloyl)piperazin-2-yl)acetonitrile

2-((S)-4-(8-Fluoro-2-(((2R, 7aS)-2-fluorotetrahydro-1H-pyrrolizin-7a(5H)-yl)methoxy)-7-(2-(trifluoromethyl)phenyl)pyrido[4,3-d]pyrimidin-4-yl)piperazin-2-yl)acetonitrile (60 mg, 0.105 mmol) and (Z)-2-fluoro-3-(thiazol-2-yl)acrylic acid (73 mg, 0.42 mmol) were combined as solids and dissolved in DMF (2.0 mL). 1-methylimidazole (67 μL, 0.84 mmol) was added followed by chloro-N,N,N′,N′-tetramethylformamidinium hexafluorophosphate (120 mg, 0.42 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (69 mg, 0.092 mmol, 88% yield). LC/MS (ESI) m/z: [M+H]⁺ calcd for C₃₄H₃₁F₆N₈O₂ S 729.2; found 729.3; ¹H NMR (500 MHz, DMSO-d₆) δ 9.18 (s, 1H), 8.05-7.99 (m, 2H), 7.94 (d, J=7.9 Hz, 1H), 7.87-7.81 (m, 1H), 7.80-7.73 (m, 1H), 7.59 (d, J=7.5 Hz, 1H), 7.09 (d, J=37.3 Hz, 1H), 5.28 (d, J=54.5 Hz, 1H), 4.96-4.84 (m, 1H), 4.57-4.49 (m, 1H), 4.48-4.37 (m, 1H), 4.16 (d, J=10.3 Hz, 1H), 4.19-4.07 (m, 1H), 4.08 (d, J=10.3 Hz, 1H), 3.87-3.74 (m, 2H), 3.14-3.06 (m, 6H), 2.87-2.79 (m, 1H), 2.17-2.10 (m, 1H), 2.08-2.03 (m, 1H), 2.02-1.97 (m, 1H), 1.87-1.74 (m, 3H).

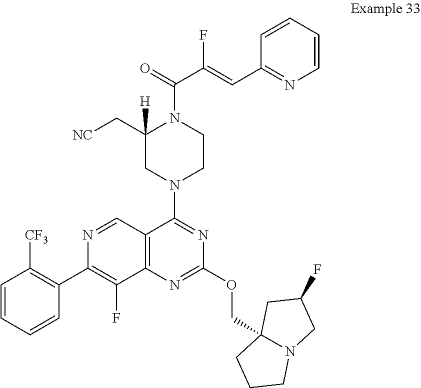 [SMILES: N#CC[C@H]1CN(c2nc(OC[C@@]34CCCN3C[C@H](F)C4)nc3c(F)c(-c4ccccc4C(F)(F)F)ncc23)CCN1C(=O)/C(F)=C/c1ccccn1]

### 2-((S)-4-(8-fluoro-2-(((2R, 7aS)-2-fluorotetrahydro-1H-pyrrolizin-7a(5H)-yl)methoxy)-7-(2-(trifluoromethyl)phenyl)pyrido[4,3-d]pyrimidin-4-yl)-1-((Z)-2-fluoro-3-(pyridin-2-yl)acryloyl)piperazin-2-yl)acetonitrile

2-((S)-4-(8-Fluoro-2-(((2R, 7aS)-2-fluorotetrahydro-1H-pyrrolizin-7a(5H)-yl)methoxy)-7-(2-(trifluoromethyl)phenyl)pyrido[4,3-d]pyrimidin-4-yl)piperazin-2-yl)acetonitrile (22 mg, 0.038 mmol) and (Z)-2-fluoro-3-(pyridin-2-yl)acrylic acid (13 mg, 0.08 mmol) were combined as solids and dissolved in DMF (0.4 mL). 1-Methylimidazole (18 μL, 0.23 mmol) was added followed by chloro-N,N,N′,N′-tetramethylformamidinium hexafluorophosphate (22 mg, 0.08 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (19 mg, 0.026 mmol, 68% yield). LC/MS (ESI) m/z: [M+H]⁺ calcd for C₃₆H₃₃F₆N₈O₂ 723.3; found 723.1; H NMR (500 MHz, DMSO-d₆) δ 9.19 (s, 1H), 8.65 (br d, J=4.3 Hz, 1H), 7.94 (d, J=8.1 Hz, 1H), 7.90 (td, J=7.8, 1.6 Hz, 1H), 7.86-7.82 (m, 1H), 7.81-7.74 (m, 2H), 7.59 (d, J=7.6 Hz, 1H), 7.39 (dd, J=7.1, 5.2 Hz, 1H), 6.66 (d, J=39.0 Hz, 1H), 5.29 (d, J=55.4 Hz, 1H), 4.97-4.85 (m, 1H), 4.58-4.49 (m, 1H), 4.49-4.39 (m, 1H), 4.26-4.03 (m, 3H), 3.87-3.73 (m, 2H), 3.27-3.15 (m, 3H), 3.15-3.03 (m, 3H), 2.92-2.81 (m, 1H), 2.22-1.97 (m, 3H), 1.89-1.73 (m, 3H).

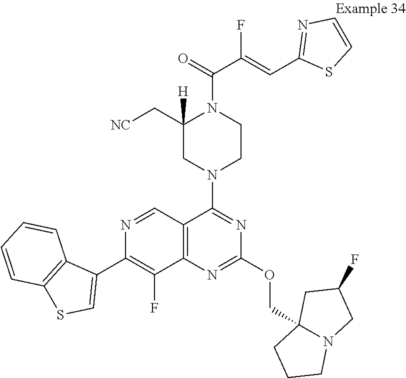 [SMILES: N#CC[C@H]1CN(c2nc(OC[C@@]34CCCN3C[C@H](F)C4)nc3c(F)c(-c4csc5ccccc45)ncc23)CCN1C(=O)/C(F)=C/c1nccs1]

### 2-((S)-4-(7-(benzo[b]thiophen-3-yl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(thiazol-2-yl)acryloyl)piperazin-2-yl)acetonitrile

2-((S)-4-(7-(Benzo[b]thiophen-3-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (22 mg, 0.039 mmol) and (Z)-2-fluoro-3-(thiazol-2-yl)acrylic acid (14 mg, 0.08 mmol) were combined as solids and dissolved in DMF (0.4 mL). 1-Methylimidazole (19 μL, 0.24 mmol) was added followed by chloro-N,N,N′,N′-tetramethylformamidinium hexafluorophosphate (22 mg, 0.08 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 70% 5:95 MeCN:H₂O with 10 mM AA/30% 95:5 MeCN:H₂O with 10 mM AA→100% 95:5 MeCN:H₂O with 10 mM AA; λ=220 nm) to provide the desired product (10.4 mg, 0.014 mmol, 36% yield). LC/MS (ESI) m/z: [M+H]⁺ calcd for C₃₅H₃₂F₃N₈O₂ S 717.2; found 717.2; ¹H NMR (500 MHz, DMSO-d₆) δ 9.28 (s, 1H), 8.39 (dd, J=6.4, 3.0 Hz, 1H), 8.37 (s, 1H), 8.14-8.09 (m, 1H), 8.05-8.02 (m, 1H), 8.02-8.00 (m, 1H), 7.50-7.44 (m, 2H), 7.11 (d, J=36.7 Hz, 1H), 5.30 (d, J=54.0 Hz, 1H), 4.96-4.84 (m, 1H), 4.58-4.44 (m, 2H), 4.26-4.07 (m, 3H), 3.87-3.68 (m, 2H), 3.30-3.18 (m, 2H), 3.16-3.03 (m, 4H), 2.91-2.80 (m, 1H), 2.18-1.98 (m, 3H), 1.89-1.74 (m, 3H).

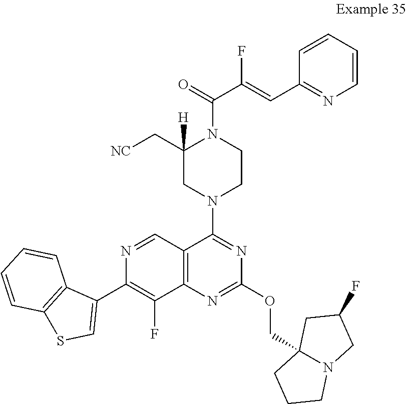 [SMILES: N#CC[C@H]1CN(c2nc(OC[C@@]34CCCN3C[C@H](F)C4)nc3c(F)c(-c4csc5ccccc45)ncc23)CCN1C(=O)/C(F)=C/c1ccccn1]

### 2-((S)-4-(7-(benzo[b]thiophen-3-yl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(pyridin-2-yl)acryloyl)piperazin-2-yl)acetonitrile

2-((S)-4-(7-(Benzo[b]thiophen-3-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (22 mg, 0.039 mmol) and (Z)-2-fluoro-3-(pyridin-2-yl)acrylic acid (13 mg, 0.08 mmol) were combined as solids and dissolved in DMF (0.4 mL). 1-methylimidazole (19 μL, 0.24 mmol) was added followed by chloro-N,N,N′,N′-tetramethylformamidinium hexafluorophosphate (22 mg, 0.08 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 71% 5:95 MeCN:H₂O with 10 mM AA/29% 95:5 MeCN:H₂O with 10 mM AA→100% 95:5 MeCN:H₂O with 10 mM AA; λ=220 nm) to provide the desired product (13.4 mg, 0.019 mmol, 48% yield). LC/MS (ESI) m/z: [M+H]⁺ calcd for C₃₇H₃₄F₃N₈O₂ S 711.3; found 717.3; ¹H NMR (500 MHz, DMSO-d₆) δ 9.28 (s, 1H), 8.66 (br d, J=4.8 Hz, 1H), 8.39 (dd, J=6.0, 3.0 Hz, 1H), 8.36 (s, 1H), 8.13-8.09 (m, 1H), 7.90 (td, J=7.8, 1.5 Hz, 1H), 7.80 (d, J=7.9 Hz, 1H), 7.49-7.44 (m, 2H), 7.39 (dd, J=6.9, 4.9 Hz, 1H), 6.66 (d, J=38.1 Hz, 1H), 5.28 (d, J=54.4 Hz, 1H), 5.00-4.83 (m, 1H), 4.59-4.46 (m, 2H), 4.19 (d, J=10.4 Hz, 1H), 4.21-4.08 (m, 1H), 4.10 (d, J=10.4 Hz, 1H), 3.87-3.68 (m, 2H), 3.19-3.00 (m, 6H), 2.88-2.80 (m, 1H), 2.17-1.99 (m, 3H), 1.89-1.75 (m, 3H).

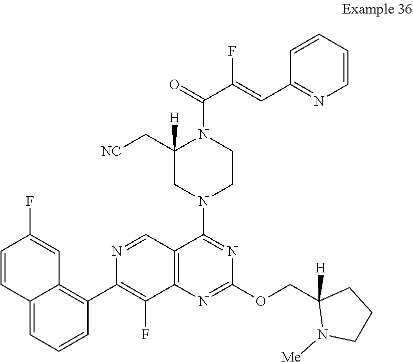 [SMILES: CN1CCC[C@H]1COc1nc(N2CCN(C(=O)/C(F)=C/c3ccccn3)[C@@H](CC#N)C2)c2cnc(-c3cccc4ccc(F)cc34)c(F)c2n1]

### 2-((S)-1-((Z)-2-fluoro-3-(pyridin-2-yl)acryloyl)-4-(8-fluoro-7-(7-fluoronaphthalen-1-yl)-2-(((S)-1-methylpyrrolidin-2-yl)methoxy)pyrido[4,3-d]pyrimidin-4-yl)piperazin-2-yl)acetonitrile

2-((S)-4-(8-Fluoro-7-(7-fluoronaphthalen-1-yl)-2-(((S)-1-methylpyrrolidin-2-yl)methoxy)pyrido[4,3-d]pyrimidin-4-yl)piperazin-2-yl)acetonitrile (19 mg, 0.036 mmol) and (Z)-2-fluoro-3-(pyridin-2-yl)acrylic acid (12 mg, 0.07 mmol) were combined as solids and dissolved in DMF (0.75 mL). 1-Methylimidazole (29 μL, 0.36 mmol) was added followed by chloro-N,N,N′,N′-tetramethylformamidinium hexafluorophosphate (20 mg, 0.07 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product (14 mg, 0.02 mmol, 58% yield). LC/MS (ESI) m/z: [M+H]⁺ calcd for C₃₇H₃₄F₃N₈O₂ 679.3; found 697.3; ¹H NMR (500 MHz, DMSO-d₆) δ 9.29 (s, 1H), 8.65 (br d, J=3.0 Hz, 1H), 8.19-8.13 (m, 2H), 7.93-7.87 (m, 1H), 7.80 (d, J=7.9 Hz, 1H), 7.76 (br d, J=6.9 Hz, 1H), 7.69-7.64 (m, 1H), 7.54-7.49 (m, 1H), 7.48-7.43 (m, 1H), 7.41-7.36 (m, 1H), 6.68 (d, J=37.7 Hz, 1H), 5.00-4.84 (m, 1H), 4.59-4.48 (m, 2H), 4.44 (br dd, J=10.7, 4.7 Hz, 1H), 4.27 (dd, J=10.7, 6.1 Hz, 1H), 4.22-4.09 (m, 1H), 3.88-3.76 (m, 3H), 3.13-3.09 (m, 1H), 2.99-2.92 (m, 1H), 2.66-2.55 (m, 2H), 2.37 (s, 3H), 2.23-2.14 (m, 1H), 1.99-1.92 (m, 1H), 1.71-1.60 (m, 3H).

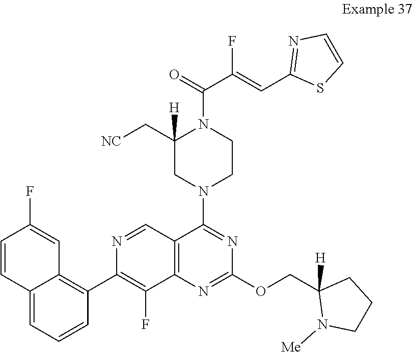 [SMILES: CN1CCC[C@H]1COc1nc(N2CCN(C(=O)/C(F)=C/c3nccs3)[C@@H](CC#N)C2)c2cnc(-c3cccc4ccc(F)cc34)c(F)c2n1]

### 2-((S)-1-((Z)-2-fluoro-3-(thiazol-2-yl)acryloyl)-4-(8-fluoro-7-(7-fluoronaphthalen-1-yl)-2-(((S)-1-methylpyrrolidin-2-yl)methoxy)pyrido[4,3-d]pyrimidin-4-yl)piperazin-2-yl)acetonitrile

2-((S)-4-(8-luoro-7-(7-Fluoronaphthalen-1-yl)-2-(((S)-1-methylpyrrolidin-2-yl)methoxy)pyrido[4,3-d]pyrimidin-4-yl)piperazin-2-yl)acetonitrile (19 mg, 0.036 mmol) and (Z)-2-fluoro-3-(thiazol-2-yl)acrylic acid (12 mg, 0.07 mmol) were combined as solids and dissolved in DMF (0.75 mL). 1-Methylimidazole (29 μL, 0.36 mmol) was added followed by chloro-N,N,N′,N′-tetramethylformamidinium hexafluorophosphate (20 mg, 0.07 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product (15.6 mg, 0.02 mmol, 64% yield). LC/MS (ESI) m/z: [M+H]⁺ calcd for C₃₅H₃₂F₃N₈O₂ S 685.3; found 685.3; ¹H NMR (500 MHz, DMSO-d₆) δ 9.29 (s, 1H), 8.21-8.12 (m, 2H), 8.05-7.97 (m, 2H), 7.76 (br d, J=7.4 Hz, 1H), 7.69-7.64 (m, 1H), 7.54-7.49 (m, 1H), 7.49-7.43 (m, 1H), 7.11 (d, J=37.5 Hz, 1H), 5.01-4.83 (m, 1H), 4.57-4.48 (m, 2H), 4.44 (dd, J=10.7, 4.6 Hz, 1H), 4.27 (dd, J=10.7, 6.1 Hz, 1H), 4.22-4.09 (m, 1H), 3.88-3.78 (m, 2H), 3.15-3.09 (m, 2H), 3.01-2.93 (m, 2H), 2.66-2.57 (m, 1H), 2.37 (s, 3H), 2.23-2.15 (m, 1H), 2.00-1.93 (m, 1H), 1.73-1.60 (m, 3H).

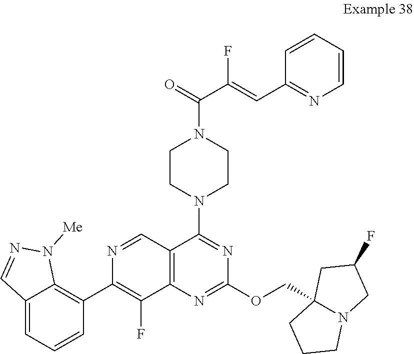 [SMILES: Cn1ncc2cccc(-c3ncc4c(N5CCN(C(=O)/C(F)=C/c6ccccn6)CC5)nc(OC[C@@]56CCCN5C[C@H](F)C6)nc4c3F)c21]

### (Z)-2-fluoro-1-(4-(8-fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)piperazin-1-yl)-3-(pyridin-2-yl)prop-2-en-1-one

8-Fluoro-2-(((2R, 7aS)-2-fluorotetrahydro-1H-pyrrolizin-7a(5H)-yl)methoxy)-7-(1-methyl-1H-indazol-7-yl)-4-(piperazin-1-yl)pyrido[4,3-d]pyrimidine (12 mg, 0.023 mmol) and (Z)-2-fluoro-3-(pyridin-2-yl)acrylic acid (7.7 mg, 0.046 mmol) were combined as solids and dissolved in DMF (0.5 mL). 1-Methylimidazole (18 μL, 0.23 mmol) was added followed by chloro-N,N,N′,N′-tetramethylformamidinium hexafluorophosphate (13 mg, 0.046 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 81% 5:95 MeCN:H₂O with 10 mM AA/19% 95:5 MeCN:H₂O with 10 mM AA→100% 95:5 MeCN:H₂O with 10 mM AA; λ=220 nm) to provide the desired product (8.2 mg, 0.012 mmol, 53% yield). LC/MS (ESI) m/z: [M+H]⁺ calcd for C₃₅H₃₅F₃N₉O₂ 670.3; found 670.2; ¹H NMR (500 MHz, DMSO-d₆) δ 9.25 (s, 1H), 8.65 (br d, J=4.0 Hz, 1H), 8.19 (s, 1H), 7.94 (d, J=8.4 Hz, 1H), 7.91-7.87 (m, 1H), 7.79 (d, J=7.9 Hz, 1H), 7.48 (d, J=6.9 Hz, 1H), 7.38 (dd, J=7.2, 5.0 Hz, 1H), 7.28 (t, J=7.6 Hz, 1H), 6.66 (d, J=38.9 Hz, 1H), 5.33 (d, J=53.7 Hz, 1H), 4.28-4.07 (m, 7H), 4.00-3.83 (m, 4H), 3.64 (s, 3H), 3.24-3.18 (m, 1H), 3.15-3.06 (m, 1H), 2.94-2.83 (m, 1H), 2.25-2.03 (m, 3H), 1.95-1.75 (m, 3H).

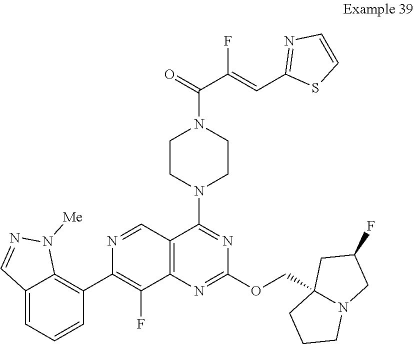 [SMILES: Cn1ncc2cccc(-c3ncc4c(N5CCN(C(=O)/C(F)=C/c6nccs6)CC5)nc(OC[C@@]56CCCN5C[C@H](F)C6)nc4c3F)c21]

### (Z)-2-fluoro-1-(4-(8-fluoro-2-(((2R, 7aS)-2-fluorotetrahydro-1H-pyrrolizin-7a(5H)-yl)methoxy)-7-(I-methyl-1H-indazol-7-yl)pyrido[4,3-d]pyrimidin-4-yl)piperazin-1-yl)-3-(thiazol-2-yl)prop-2-en-1-one

8-Fluoro-2-(((2R, 7aS)-2-fluorotetrahydro-1H-pyrrolizin-7a(5H)-yl)methoxy)-7-(1-methyl-1H-indazol-7-yl)-4-(piperazin-1-yl)pyrido[4,3-d]pyrimidine (12 mg, 0.023 mmol) and (Z)-2-fluoro-3-(thiazol-2-yl)acrylic acid (8 mg, 0.046 mmol) were combined as solids and dissolved in DMF (0.5 mL). 1-Methylimidazole (18 μL, 0.23 mmol) was added followed by chloro-N,N,N′,N′-tetramethylformamidinium hexafluorophosphate (13 mg, 0.046 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (11 mg, 0.016 mmol, 70% yield). LC/MS (ESI) m/z: [M+H]⁺ calcd for C₃₃H₃₃F₃N₉O₂ S 676.2; found 676.3; ¹H NMR (500 MHz, DMSO-d₆) δ 9.25 (s, 1H), 8.19 (s, 1H), 8.04-7.98 (m, 2H), 7.96-7.91 (m, 1H), 7.50-7.46 (m, 1H), 7.31-7.25 (m, 1H), 0.00 (d, J=37.5 Hz, 1H), 5.30 (d, J=54.2 Hz, 1H), 4.27-4.06 (m, 7H), 4.00-3.84 (m, 4H), 3.64 (s, 3H), 3.15-3.01 (m, 2H), 2.91-2.82 (m, 1H), 2.22-2.01 (m, 3H), 1.90-1.74 (m, 3H).

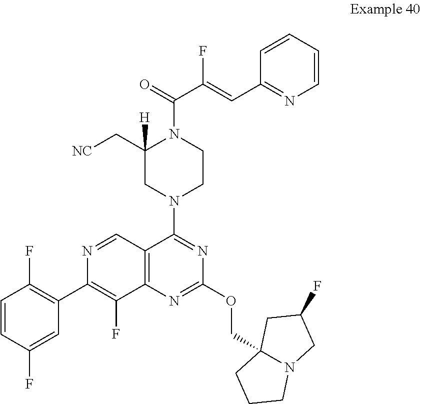 [SMILES: N#CC[C@H]1CN(c2nc(OC[C@@]34CCCN3C[C@H](F)C4)nc3c(F)c(-c4cc(F)ccc4F)ncc23)CCN1C(=O)/C(F)=C/c1ccccn1]

### 2-((S)-4-(7-(2,5-difluorophenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(pyridin-2-yl)acryloyl)piperazin-2-yl)acetonitrile

2-((S)-4-(7-(2,5-Difluorophenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (15 mg, 0.028 mmol) and (Z)-2-fluoro-3-(pyridin-2-yl)acrylic acid (9.3 mg, 0.055 mmol) were combined as solids and dissolved in DMF (0.55 mL). 1-Methylimidazole (13 μL, 0.17 mmol) was added followed by chloro-N,N,N′,N′-tetramethylformamidinium hexafluorophosphate (16 mg, 0.055 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product (11.3 mg, 0.016 mmol, 58% yield). LC/MS (ESI) m/z: [M+H]⁺ calcd for C₃₅H₃₂F₅N₈O₂ 691.3; found 691.2; ¹H NMR (500 MHz, DMSO-d₆) δ 9.22 (s, 1H), 8.65 (br d, J=3.7 Hz, 1H), 7.93-7.86 (m, 1H), 7.79 (d, J=8.1 Hz, 1H), 7.56-7.50 (m, 1H), 7.50-7.44 (m, 2H), 7.41-7.36 (m, 1H), 0.00 (d, J=38.8 Hz, 1H), 5.28 (d, J=55.0 Hz, 1H), 4.98-4.80 (m, 1H), 4.59-4.50 (m, 1H), 4.48-4.37 (m, 1H), 4.17 (d, J=10.4 Hz, 1H), 4.14-4.05 (m, 2H), 4.09 (d, J=10.4 Hz, 1H), 3.87-3.67 (m, 2H), 3.16-3.01 (m, 5H), 2.88-2.79 (m, 1H), 2.20-1.98 (m, 3H), 1.88-1.73 (m, 3H).

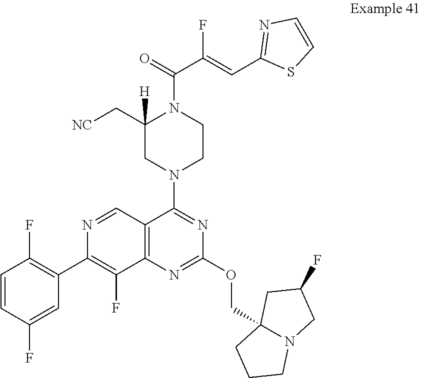 [SMILES: N#CC[C@H]1CN(c2nc(OC[C@@]34CCCN3C[C@H](F)C4)nc3c(F)c(-c4cc(F)ccc4F)ncc23)CCN1C(=O)/C(F)=C/c1nccs1]

### 2-((S)-4-(7-(2,5-difluorophenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(thiazol-2-yl)acryloyl)piperazin-2-yl)acetonitrile

2-((S)-4-(7-(2,5-Difluorophenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (15 mg, 0.028 mmol) and (Z)-2-fluoro-3-(thiazol-2-yl)acrylic acid (9.6 mg, 0.055 mmol) were combined as solids and dissolved in DMF (0.55 mL). 1-Methylimidazole (13 μL, 0.17 mmol) was added followed by chloro-N,N,N′,N′-tetramethylformamidinium hexafluorophosphate (16 mg, 0.055 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 78% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (0.8 mg, 0.015 mmol, 56% yield).LC/MS (ESI) m/z: [M+H]⁺ calcd for C₃₃H₃₀F₅NsO₂ S 697.2; found 697.2; ¹H NMR (500 MHz, DMSO-d₆) δ 9.22 (s, 1H), 8.07-7.97 (m, 2H), 7.57-7.50 (m, 1H), 7.50-7.44 (m, 2H), 7.10 (d, J=38.0 Hz, 1H), 5.28 (d, J=54.6 Hz, 1H), 4.95-4.81 (m, 1H), 4.58-4.48 (m, 1H), 4.48-4.37 (m, 1H), 4.22-4.04 (m, 4H), 3.87-3.70 (m, 2H), 3.15-3.00 (m, 5H), 2.88-2.78 (m, 1H), 2.15-2.00 (m, 3H), 1.87-1.75 (m, 3H).

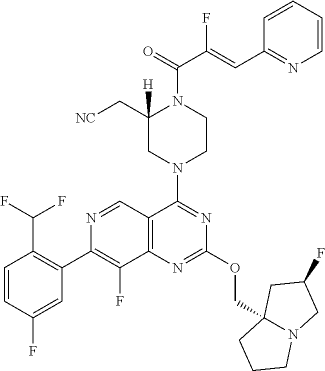 [SMILES: N#CC[C@H]1CN(c2nc(OC[C@@]34CCCN3C[C@H](F)C4)nc3c(F)c(-c4cc(F)ccc4C(F)F)ncc23)CCN1C(=O)/C(F)=C/c1ccccn1]

### Example 42

### 2-((S)-4-(7-(2-(difluoromethyl)-5-fluorophenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(pyridin-2-yl)acryloyl)piperazin-2-yl)acetonitrile

2-((S)-4-(7-(2-(Difluoromethyl)-5-fluorophenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (15 mg, 0.026 mmol) and (Z)-2-fluoro-3-(pyridin-2-yl)acrylic acid (8.7 mg, 0.052 mmol) were combined as solids and dissolved in DMF (1.3 mL). 1-Methylimidazole (13 μL, 0.16 mmol) was added followed by chloro-N,N,N′,N′-tetramethylformamidinium hexafluorophosphate (15 mg, 0.052 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 73% 5:95 MeCN:H₂O with 10 mM AA/27% 95:5 MeCN:H₂O with 10 mM AA→100% 95:5 MeCN:H₂O with 10 mM AA; λ=220 nm) to provide the desired product (11.9 mg, 0.016 mmol, 62% yield).LC/MS (ESI) m/z: [M+H]⁺ calcd for C₃₆H₃₃F₆N₈O₂ 723.3; found 723.3;¹H NMR (500 MHz, DMSO-d₆) δ 9.20 (s, 1H), 8.66 (br d, J=4.3 Hz, 1H), 7.93-7.87 (m, 2H), 7.82-7.74 (m, 1H), 7.60-7.51 (m, 2H), 7.39 (dd, J=6.9, 5.0 Hz, 1H), 7.03 (t, J=55.0 Hz, 1H), 6.66 (d, J=39.8 Hz, 1H), 5.28 (d, J=54.8 Hz, 1H), 4.97-4.81 (m, 1H), 4.56-4.41 (m, 2H), 4.21-4.05 (m, 3H), 3.87-3.61 (m, 2H), 3.27-3.20 (m, 1H), 3.16-2.99 (m, 4H), 2.91-2.76 (m, 1H), 2.21-1.96 (m, 3H), 1.88-1.70 (m, 3H).

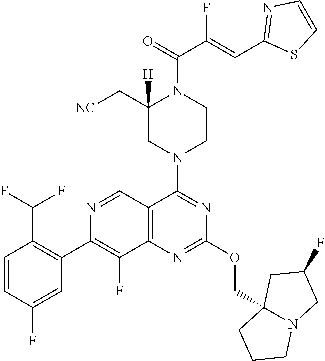 [SMILES: N#CC[C@H]1CN(c2nc(OC[C@@]34CCCN3C[C@H](F)C4)nc3c(F)c(-c4cc(F)ccc4C(F)F)ncc23)CCN1C(=O)/C(F)=C/c1nccs1]

### Example 43

### 2-((S)-4-(7-(2-(difluoromethyl)-5-fluorophenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(thiazol-2-yl)acryloyl)piperazin-2-yl)acetonitrile

2-((S)-4-(7-(2-(Difluoromethyl)-5-fluorophenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (15 mg, 0.026 mmol) and (Z)-2-fluoro-3-(thiazol-2-yl)acrylic acid (9.1 mg, 0.052 mmol) were combined as solids and dissolved in DMF (1.3 mL). 1-Methylimidazole (13 μL, 0.16 mmol) was added followed by chloro-N,N,N′,N′-tetramethylformamidinium hexafluorophosphate (15 mg, 0.052 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 72% 5:95 MeCN:H₂O with 10 mM AA/28% 95:5 MeCN:H₂O with 10 mM AA→100% 95:5 MeCN:H₂O with 10 mM AA; λ=220 nm) to provide the desired product (8.2 mg, 0.011 mmol, 43% yield). LC/MS (ESI) m/z: [M+H]⁺ calcd for C₃₄H₃₁F₆N₈O₂ S 729.2; found 729.1; ¹H NMR (500 MHz, DMSO-d₆) δ 9.20 (s, 1H), 8.05-7.97 (m, 2H), 7.94-7.84 (m, 1H), 7.61-7.49 (m, 2H), 7.10 (d, J=37.3 Hz, 1H), 7.03 (t, J=54.9 Hz, 1H), 5.28 (d, J=53.9 Hz, 1H), 5.00-4.84 (m, 1H), 4.51 (br s, 2H), 4.25-4.05 (m, 4H), 3.87-3.67 (m, 2H), 3.27-3.21 (m, 1H), 3.12-2.99 (m, 4H), 2.88-2.73 (m, 1H), 2.15-1.98 (m, 3H), 1.87-1.69 (m, 3H).

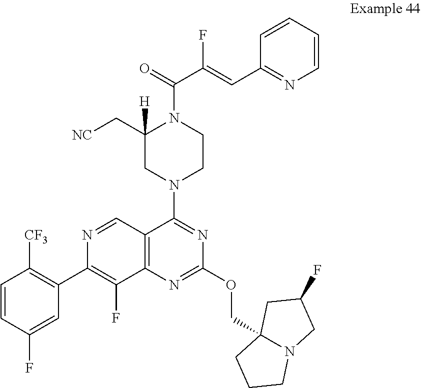 [SMILES: N#CC[C@H]1CN(c2nc(OC[C@@]34CCCN3C[C@H](F)C4)nc3c(F)c(-c4cc(F)ccc4C(F)(F)F)ncc23)CCN1C(=O)/C(F)=C/c1ccccn1]

### 2-((S)-1-((Z)-2-fluoro-3-(pyridin-2-yl)acryloyl)-4-(8-fluoro-7-(5-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

2-((S)-4-(8-Fluoro-7-(5-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (15 mg, 0.025 mmol) and (Z)-2-fluoro-3-(pyridin-2-yl)acrylic acid (8.5 mg, 0.052 mmol) were combined as solids and dissolved in DMF (1.3 mL). 1-Methylimidazole (12 μL, 0.15 mmol) was added followed by chloro-N,N,N′,N′-tetramethylformamidinium hexafluorophosphate (14 mg, 0.051 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 73% 5:95 MeCN:H₂O with 10 mM AA/27% 95:5 MeCN:H₂O with 10 mM AA→100% 95:5 MeCN:H₂O with 10 mM AA; λ=220 nm) to provide the desired product (14.1 mg, 0.019 mmol, 72% yield). LC/MS (ESI) m/z: [M+H]⁺ calcd for C₃₆H₃₂F₇N₈O₂ 741.3; found 741.0; ¹H NMR (500 MHz, DMSO-d₆) δ 9.18 (s, 1H), 8.65 (br d, J=4.0 Hz, 1H), 8.03 (dd, J=8.9, 5.2 Hz, 1H), 7.93-7.85 (m, 1H), 7.79 (d, J=7.9 Hz, 1H), 7.66-7.59 (m, 1H), 7.56 (br d, J=7.5 Hz, 1H), 7.39 (dd, J=6.9, 5.3 Hz, 1H), 6.66 (d, J=38.5 Hz, 1H), 5.29 (d, J=54.0 Hz, 1H), 4.98-4.83 (m, 1H), 4.60-4.49 (m, 1H), 4.47-4.38 (m, 1H), 4.16 (d, J=10.2 Hz, 1H), 4.09 (d, J=10.2 Hz, 1H), 4.19-4.05 (m, 1H), 3.87-3.65 (m, 2H), 3.24-3.18 (m, 1H), 3.20-3.01 (m, 4H), 2.87-2.77 (m, 1H), 2.17-1.96 (m, 3H), 1.88-1.72 (m, 3H).

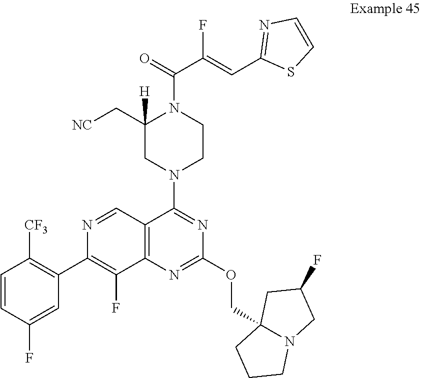 [SMILES: N#CC[C@H]1CN(c2nc(OC[C@@]34CCCN3C[C@H](F)C4)nc3c(F)c(-c4cc(F)ccc4C(F)(F)F)ncc23)CCN1C(=O)/C(F)=C/c1nccs1]

### 2-((S)-1-((Z)-2-fluoro-3-(thiazol-2-yl)acryloyl)-4-(8-fluoro-7-(5-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

2-((S)-4-(8-Fluoro-7-(5-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (15 mg, 0.025 mmol) and (Z)-2-fluoro-3-(thiazol-2-yl)acrylic acid (8.7 mg, 0.051 mmol) were combined as solids and dissolved in DMF (1.3 mL). 1-Methylimidazole (12 μL, 0.15 mmol) was added followed by chloro-N,N,N′,N′-tetramethylformamidinium hexafluorophosphate (14 mg, 0.051 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 72% 5:95 MeCN:H₂O with 10 mM AA/28% 95:5 MeCN:H₂O with 10 mM AA→100% 95:5 MeCN:H₂O with 10 mM AA; λ=220 nm) to provide the desired product (13.7 mg, 0.017 mmol, 68% yield).LC/MS (ESI) m/z: [M+H]⁺ calcd for C₃₄H₃₀F₇NsO₂ S 747.2; found 747.1; ¹H NMR (500 MHz, DMSO-d₆) δ 9.18 (s, 1H), 8.08-7.98 (m, 3H), 7.69-7.60 (m, 1H), 7.58-7.53 (m, 1H), 7.10 (d, J=38.2 Hz, 1H), 5.27 (d, J=55.2 Hz, 1H), 4.95-4.84 (m, 1H), 4.52 (br d, J=13.0 Hz, 1H), 4.46-4.36 (m, 1H), 4.16 (d, J=10.6 Hz, 1H), 4.08 (d, J=10.6 Hz, 1H), 4.20-4.05 (m, 1H), 3.89-3.70 (m, 2H), 3.15-3.00 (m, 6H), 2.87-2.78 (m, 1H), 2.18-1.95 (m, 3H), 1.88-1.72 (m, 3H).

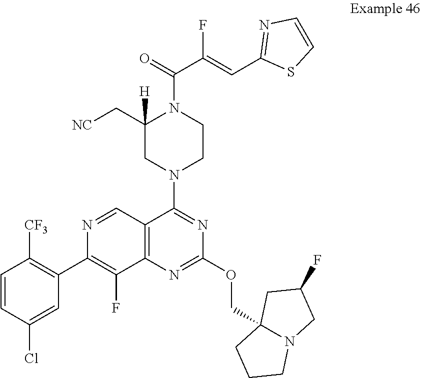 [SMILES: N#CC[C@H]1CN(c2nc(OC[C@@]34CCCN3C[C@H](F)C4)nc3c(F)c(-c4cc(Cl)ccc4C(F)(F)F)ncc23)CCN1C(=O)/C(F)=C/c1nccs1]

### 2-((S)-1-((Z)-2-fluoro-3-(thiazol-2-yl)acryloyl)-4-(8-fluoro-7-(5-chloro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

2-((S)-4-(8-Fluoro-7-(5-chloro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (15 mg, 0.025 mmol) and (Z)-2-fluoro-3-(thiazol-2-yl)acrylic acid (8.5 mg, 0.049 mmol) were combined as solids and dissolved in DMF (1.2 mL). 1-Methylimidazole (12 μL, 0.15 mmol) was added followed by chloro-N,N,N′,N′-tetramethylformamidinium hexafluorophosphate (14 mg, 0.049 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 68% 5:95 MeCN:H₂O with 10 mM AA/32% 95:5 MeCN:H₂O with 10 mM AA→100% 95:5 MeCN:H₂O with 10 mM AA; λ=220 nm) to provide the desired product (6 mg, 0.017 mmol, 30% yield).LC/MS (ESI) m/z: [M+H]⁺ calcd for C₃₄H₃₀ClF₆NsO₂ S 763.2; found 763.0; ¹H NMR (500 MHz, DMSO-d₆) δ 9.18 (s, 1H), 8.05-8.00 (m, J=6.3 Hz, 2H), 8.00-7.96 (m, 1H), 7.86 (br d, J=8.0 Hz, 1H), 7.76 (s, 1H), 7.09 (d, J=36.6 Hz, 1H), 5.28 (d, J=54.3 Hz, 1H), 4.99-4.84 (m, 1H), 4.57-4.50 (m, 1H), 4.48-4.39 (m, 1H), 4.17 (d, J=10.6 Hz, 1H), 4.09 (d, J=10.6 Hz, 1H), 4.20-4.05 (m, 1H), 3.88-3.70 (m, 2H), 3.25-2.96 (m, 6H), 2.86-2.77 (m, 1H), 2.18-1.98 (m, 3H), 1.89-1.74 (m, 3H).

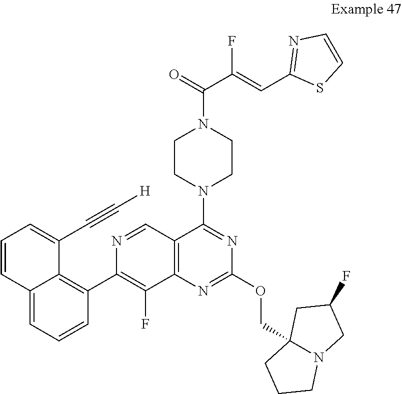 [SMILES: C#Cc1cccc2cccc(-c3ncc4c(N5CCN(C(=O)/C(F)=C/c6nccs6)CC5)nc(OC[C@@]56CCCN5C[C@H](F)C6)nc4c3F)c12]

### (Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(thiazol-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (10 mg, 0.018 mmol), and (Z)-2-fluoro-3-(thiazol-2-yl)acrylic acid (6.4 mg, 0.037 mmol) were combined as solids and dissolved in DMF (930 μL). 1-Methylimidazole (9 μL, 0.11 mmol) was added followed by chloro-N,N,N′,N′-tetramethylformamidinium hexafluorophosphate (10 mg, 0.037 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide the desired product (12 mg, 0.017 mmol, 92% yield) as a brown solid. LC/MS (ESI) m/z: [M+H]⁺ calcd for C₃₇H₃₂F₃N₇O₂ S 696.2; found 696.2; ¹H NMR (500 MHz, DMSO-d₆) δ 9.12 (s, 1H), 8.14 (dd, J=12.2, 8.3 Hz, 2H), 8.01 (br d, J=9.3 Hz, 2H), 7.74-7.66 (m, 2H), 7.64-7.53 (m, 2H), 7.11 (d, J=37.3 Hz, 1H), 5.28 (d, J=54.9 Hz, 1H), 4.19-3.91 (m, 9H), 3.71 (s, 1H), 3.08 (br d, J=11.9 Hz, 3H), 2.87-2.79 (m, 1H), 2.14-1.98 (m, 3H), 1.88-1.74 (m, 3H).

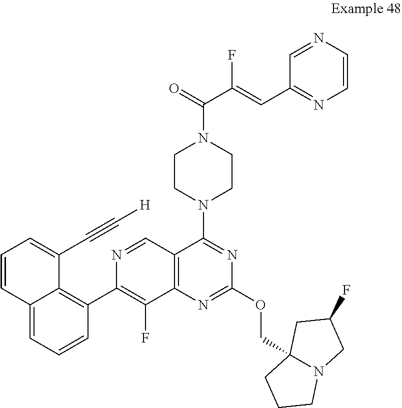 [SMILES: C#Cc1cccc2cccc(-c3ncc4c(N5CCN(C(=O)/C(F)=C/c6cnccn6)CC5)nc(OC[C@@]56CCCN5C[C@H](F)C6)nc4c3F)c12]

### (Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridazin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (6 mg, 0.011 mmol), and (Z)-2-fluoro-3-(pyridazin-2-yl)acrylic acid (3.7 mg, 0.022 mmol) were combined as solids and dissolved in DMF (550 μL). 1-Methylimidazole (5 μL, 0.067 mmol) was added followed by chloro-N,N,N′,N′-tetramethylformamidinium hexafluorophosphate (6.2 mg, 0.022 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 78% 5:95 MeCN:H₂O with 10 mM AA/22% 95:5 MeCN:H₂O with 10 mM AA→100% 95:5 MeCN:H₂O with 10 mM AA; λ=220 nm) to provide the desired product (5.9 mg, 0.008 mmol, 77% yield) as a brown solid. LC/MS (ESI) m/z: [M+H]⁺ calcd for C₃₈H₃₄F₃N₈O₂ 691.3; found 691.2; ¹H NMR (500 MHz, DMSO-d₆) δ 9.12 (s, 1H), 8.97 (s, 1H), 8.73 (br s, 1H), 8.61 (br s, 1H), 8.14 (br dd, J=11.8, 8.4 Hz, 2H), 7.75-7.66 (m, 2H), 7.65-7.51 (m, 2H), 6.75 (d, J=38.8 Hz, 1H), 5.28 (d, J=54.8 Hz, 1H), 4.20-3.97 (m, 9H), 3.71 (s, 1H), 3.11-3.00 (m, 4H), 2.87-2.79 (m, 1H), 2.12 (br s, 1H), 2.07 (br d, J=2.9 Hz, 1H), 2.04-1.97 (m, 1H), 1.87-1.71 (m, 3H).

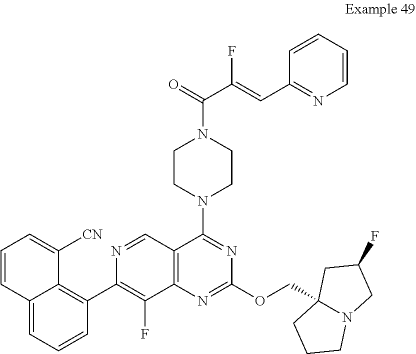 [SMILES: N#Cc1cccc2cccc(-c3ncc4c(N5CCN(C(=O)/C(F)=C/c6ccccn6)CC5)nc(OC[C@@]56CCCN5C[C@H](F)C6)nc4c3F)c12]

### 8-(8-fluoro-4-(4-((Z)-2-fluoro-3-(pyridin-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7yl)-1-naphthonitrile

8-(8-Fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)-1-naphthonitrile (5 mg, 9.2 μmol) and (Z)-2-fluoro-3-(pyridin-2-yl)acrylic acid (3.1 mg, 0.018 mmol) were combined as solids and dissolved in DMF (500 μL). 1-Methylimidazole (7 μL, 0.092 mmol) was added followed by chloro-N,N,N′,N′-tetramethylformamidinium hexafluorophosphate (5.2 mg, 0.018 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (4.7 mg, 0.006 mmol, 74% yield).LC/MS (ESI) m/z: [M+H]⁺ calcd for C₃₈H₃₄F₃N₈O₂ 691.3; found 691.2; ¹H NMR (500 MHz, DMSO-d₆) δ 9.21 (s, 1H), 8.64 (br s, 1H), 8.46 (br d, J=8.4 Hz, 1H), 8.32-8.27 (m, 1H), 8.14 (br d, J=7.2 Hz, 1H), 7.91-7.86 (m, 1H), 7.86-7.82 (m, 1H), 7.81-7.77 (m, 2H), 7.77-7.71 (m, 1H), 7.41-7.35 (m, 1H), 6.65 (d, J=38.6 Hz, 1H), 5.28 (d, J=54.0 Hz, 1H), 4.20-4.06 (m, 7H), 4.03-3.82 (m, 2H), 3.21-3.00 (m, 4H), 2.87-2.78 (m, 1H), 2.16-1.98 (m, 3H), 1.88-1.73 (m, 3H).

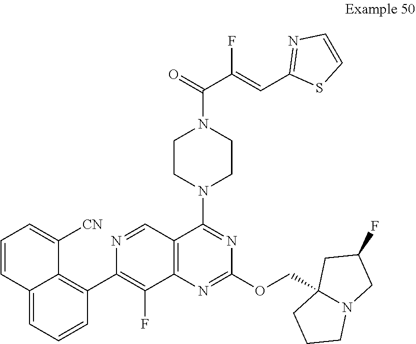 [SMILES: N#Cc1cccc2cccc(-c3ncc4c(N5CCN(C(=O)/C(F)=C/c6nccs6)CC5)nc(OC[C@@]56CCCN5C[C@H](F)C6)nc4c3F)c12]

### 8-(8-fluoro-4-(4-((Z)-2-fluoro-3-(thiazol-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7yl)-1-naphthonitrile

8-(8-Fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)-1-naphthonitrile (5 mg, 9.2 μmol), and (Z)-2-fluoro-3-(thiazol-2-yl)acrylic acid (3.2 mg, 0.018 mmol) were combined as solids and dissolved in DMF (500 μL). 1-Methylimidazole (7 μL, 0.092 mmol) was added followed by chloro-N,N,N′,N′-tetramethylformamidinium hexafluorophosphate (5.2 mg, 0.018 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (5.3 mg, 0.007 mmol, 81% yield). LC/MS (ESI) m/z: [M+H]⁺ calcd for C₃₆H₃₂F₃N₈O₂ S 697.2; found 697.0; ¹H NMR (500 MHz, DMSO-d₆) δ 9.21 (s, 1H), 8.46 (br d, J=8.2 Hz, 1H), 8.29 (br d, J=7.9 Hz, 1H), 8.14 (br d, J=7.1 Hz, 1H), 8.04-7.96 (m, 2H), 7.87-7.81 (m, 1H), 7.81-7.78 (m, 1H), 7.77-7.70 (m, 1H), 7.10 (d, J=37.5 Hz, 1H), 5.28 (d, J=53.9 Hz, 1H), 4.22-4.07 (m, 7H), 4.02-3.86 (m, 2H), 3.20-3.02 (m, 4H), 2.87-2.79 (m, 1H), 2.18-2.00 (m, 3H), 1.89-1.73 (m, 3H).

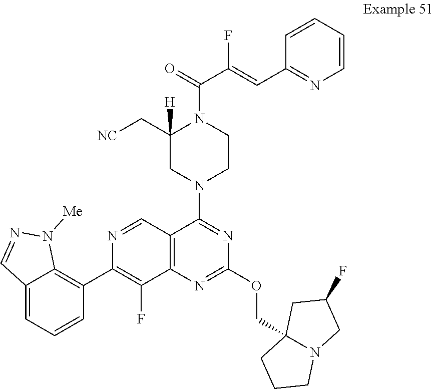 [SMILES: Cn1ncc2cccc(-c3ncc4c(N5CCN(C(=O)/C(F)=C/c6ccccn6)[C@@H](CC#N)C5)nc(OC[C@@]56CCCN5C[C@H](F)C6)nc4c3F)c21]

### 2-((S)-4-(8-fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)-1-((Z)-2-fluoro-3-(pyridin-2-yl)acryloyl)piperazin-2-yl)acetonitrile

2-((S)-4-(8-Fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)piperazin-2-yl)acetonitrile (24 mg, 0.043 mmol) and (Z)-2-fluoro-3-(pyridin-2-yl)acrylic acid (14 mg, 0.086 mmol) were combined as solids and dissolved in DMF (1.0 mL). 1-Methylimidazole (34 μL, 0.43 mmol) was added followed by chloro-N,N,N′,N′-tetramethylformamidinium hexafluorophosphate (24 mg, 0.086 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (16.9 mg, 0.023 mmol, 56% yield). LC/MS (ESI) m/z: [M+H]⁺ calcd for C₃₇H₃₆F₃N₁₀O₂ 709.3; found 709.2; ¹H NMR (500 MHz, DMSO-d₆) δ 9.26 (s, 1H), 8.65 (br d, J=4.1 Hz, 1H), 8.19 (s, 1H), 7.94 (d, J=8.1 Hz, 1H), 7.92-7.88 (m, 1H), 7.80 (d, J=8.0 Hz, 1H), 7.48 (d, J=7.1 Hz, 1H), 7.42-7.36 (m, 1H), 7.29 (t, J=7.6 Hz, 1H), 6.66 (d, J=37.9 Hz, 1H), 5.28 (d, J=54.2 Hz, 1H), 4.98-4.86 (m, 1H), 4.58-4.52 (m, 1H), 4.52-4.44 (m, 1H), 4.18 (d, J=10.4 Hz, 1H), 4.10 (br d, J=10.4 Hz, 1H), 4.22-4.06 (m, 1H), 3.89-3.71 (m, 2H), 3.65 (s, 3H), 3.19-3.01 (m, 6H), 2.87-2.79 (m, 1H), 2.07 (br d, J=1.8 Hz, 3H), 1.88-1.72 (m, 3H).

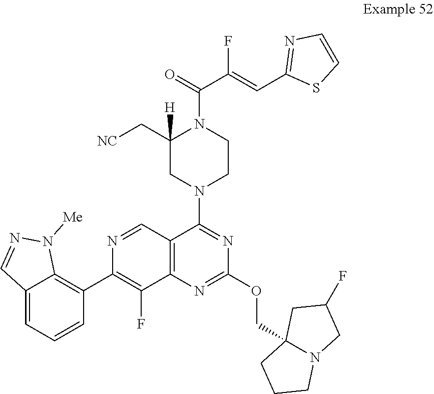 [SMILES: Cn1ncc2cccc(-c3ncc4c(N5CCN(C(=O)/C(F)=C/c6nccs6)[C@@H](CC#N)C5)nc(OC[C@@]56CCCN5CC(F)C6)nc4c3F)c21]

### 2-((S)-4-(8-fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)-1-((Z)-2-fluoro-3-(thiazol-2-yl)acryloyl)piperazin-2-yl)acetonitrile

2-((S)-4-(8-Fluoro-2-(((2R, 7aS)-2-fluorotetrahydro-1H-pyrrolizin-7a(5H)-yl)methoxy)-7-(1-methyl-1H-indazol-7-yl)pyrido[4,3-d]pyrimidin-4-yl)piperazin-2-yl)acetonitrile (24 mg, 0.043 mmol) and (Z)-2-fluoro-3-(thiazol-2-yl)acrylic acid (15 mg, 0.086 mmol) were combined as solids and dissolved in DMF (1.0 mL). 1-Methylimidazole (34 μL, 0.43 mmol) was added followed by chloro-N,N,N′,N′-tetramethylformamidinium hexafluorophosphate (24 mg, 0.086 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (20.5 mg, 0.029 mmol, 67% yield). LC/MS (ESI) m/z: [M+H]⁺ calcd for C₃₅H₃₄F₃N₁₀O₂ S 715.0; found 715.3; ¹H NMR (500 MHz, DMSO-d₆) δ 9.26 (s, 1H), 8.19 (s, 1H), 8.05-7.99 (m, 2H), 7.94 (d, J=8.0 Hz, 1H), 7.48 (d, J=6.8 Hz, 1H), 7.29 (t, J=7.6 Hz, 1H), 7.11 (d, J=37.8 Hz, 1H), 5.28 (d, J=53.9 Hz, 1H), 4.97 (s, 1H), 4.60-4.53 (m, 1H), 4.51-4.44 (m, 1H), 4.18 (d, J=10.5 Hz, 1H), 4.10 (br d, J=10.5 Hz, 1H), 4.21-4.06 (m, 1H), 3.88-3.75 (m, 2H), 3.65 (s, 3H), 3.18-3.03 (m, 6H), 2.88-2.79 (m, 1H), 2.16-1.99 (m, 3H), 1.88-1.71 (m, 3H).

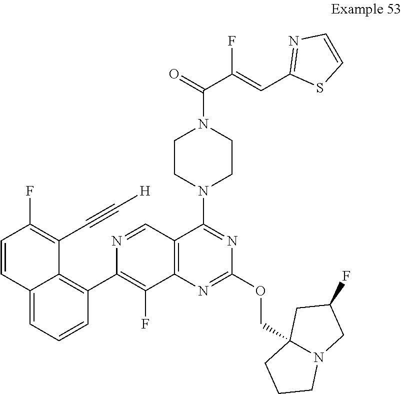

### (Z)-1-(4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(thiazol-2-yl)prop-2-en-1-one

7-(8-Ethynyl-7-fluoronaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (5 mg, 0.009 mmol) and (Z)-2-fluoro-3-(thiazol-2-yl)acrylic acid (6.2 mg, 0.036 mmol) were combined as solids and dissolved in DMF (500 μL). 1-methylimidazole (7 μL, 0.09 mmol) was added followed by chloro-N,N,N′,N′-tetramethylformamidinium hexafluorophosphate (10 mg, 0.036 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 73% 5:95 MeCN:H₂O with 10 mM AA/27% 95:5 MeCN:H₂O with 10 mM AA→100% 95:5 MeCN:H₂O with 10 mM AA; λ=220 nm) to provide the desired product (2.3 mg, 0.003 mmol, 36% yield) as a brown solid. LC/MS (ESI) m/z: [M+H]⁺ calcd for C₃₇H₃₁F₄N₇O₂ S 714.2; found 714.3. ¹H NMR (500 MHz, DMSO-d₆) δ 9.13 (s, 1H), 8.26-8.16 (m, 2H), 7.73-7.64 (m, 2H), 7.64-7.58 (m, 1H), 7.11 (d, J=37.3 Hz, 1H), 5.31 (d, J=54.3 Hz, 1H), 4.25-4.07 (m, 8H), 4.05 (s, 1H), 4.02-3.86 (m, 4H), 3.14-3.01 (m, 2H), 2.93-2.81 (m, 1H), 2.23-2.00 (m, 3H), 1.90-1.68 (m, 3H).

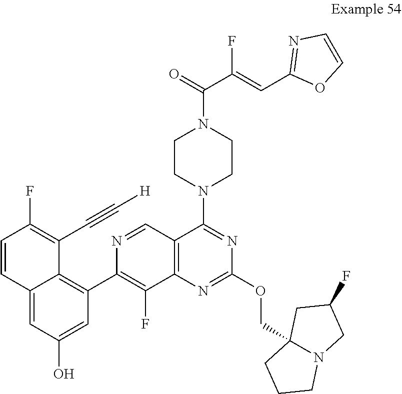

### (Z)-1-(4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(oxazol-2-yl)prop-2-en-1-one

5-Ethynyl-6-fluoro-4-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol (5 mg, 0.008 mmol) and (Z)-2-fluoro-3-(oxazol-2-yl)acrylic acid (5.5 mg, 0.035 mmol) were combined as solids and dissolved in DMF (300 μL). 1-Methylimidazole (5.6 μL, 0.07 mmol) was added followed by chloro-N,N,N′,N′-tetramethylformamidinium hexafluorophosphate (9.8 mg, 0.035 mmol). The reaction mixture was stirred for 5 min. A saturated aqueous solution of ammonium hydroxide (100 μL) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 84% 5:95 MeCN:H₂O with 10 mM AA/16% 95:5 MeCN:H₂O with 10 mM AA→100% 95:5 MeCN:H₂O with 10 mM AA; λ=220 nm) to provide the desired product (5.6 mg, 0.008 mmol, 90% yield) as a brown solid. LC/MS (ESI) m/z: [M+H]⁺ calcd for C₃₇H₃₂F₄N₇O₄ 714.2; found 714.2;¹H NMR (500 MHz, DMSO-d₆) δ 10.18 (br s, 1H), 9.11 (s, 1H), 8.27 (s, 1H), 7.97 (dd, J=8.9, 6.1 Hz, 1H), 7.49-7.44 (m, 2H), 7.40 (d, J=2.5 Hz, 1H), 7.19 (s, 1H), 6.72 (d, J=35.6 Hz, 1H), 5.30 (d, J=53.3 Hz, 1H), 4.22-4.06 (m, 8H), 3.97 (s, 1H), 3.95-3.90 (m, 2H), 3.15-3.04 (m, 3H), 2.91-2.82 (m, 1H), 2.17-2.08 (m, 2H), 2.05-2.00 (m, 1H), 1.89-1.72 (m, 3H).

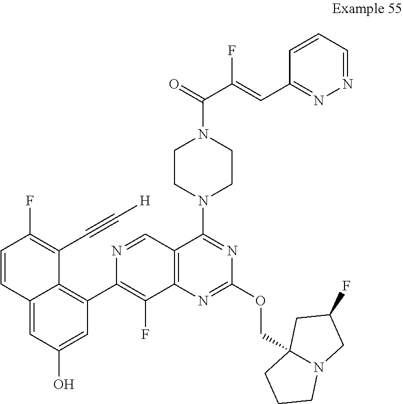

### (Z)-1-(4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridazin-3-yl)prop-2-en-1-one

5-Ethynyl-6-fluoro-4-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol (5 mg, 0.008 mmol) and (Z)-2-fluoro-3-(pyridazin-3-yl)acrylic acid (5.9 mg, 0.035 mmol) were combined as solids and dissolved in DMF (300 μL). 1-Methylimidazole (5.6 μL, 0.07 mmol) was added followed by chloro-N,N,N′,N′-tetramethylformamidinium hexafluorophosphate (9.8 mg, 0.035 mmol). The reaction mixture was stirred for 5 min. A saturated aqueous solution of ammonium hydroxide (100 μL) was added, and the reaction mixture was stirred for an additional 5 min. The reaction mixture was directly purified by reverse phase HPLC (column: Xbridge C18, 19 mm×200 mm, 5 μm particles; flow rate: 20 mL/min; column temperature: 25° C.; gradient: 87% 5:95 MeCN:H₂O with 10 mM AA/13% 95:5 MeCN:H₂O with 10 mM AA→100% 95:5 MeCN:H₂O with 10 mM AA; λ=220 nm) to provide the desired product (6 mg, 0.008 mmol, 92% yield) as a brown solid. LC/MS (ESI) m/z: [M+H]⁺ calcd for C₃₈H₃₃F₄N₈O₃ 725.3; found 725.3; ¹H NMR (500 MHz, DMSO-d₆) δ 10.18 (br s, 1H), 9.20 (dd, J=4.9, 1.6 Hz, 1H), 9.12 (s, 1H), 8.05 (dd, J=8.8, 1.4 Hz, 1H), 7.97 (dd, J=9.2, 6.0 Hz, 1H), 7.79 (dd, J=8.5, 4.8 Hz, 1H), 7.46 (t, J=9.0 Hz, 1H), 7.40 (d, J=2.3 Hz, 1H), 7.20 (s, 1H), 6.92 (d, J=38.2 Hz, 1H), 5.31 (d, J=55.2 Hz, 1H), 4.26-4.00 (m, 10H), 3.97 (s, 1H), 3.95-3.90 (m, 1H), 3.15-3.04 (m, 2H), 2.94-2.82 (m, 1H), 2.20-2.09 (m, 2H), 2.06-1.99 (m, 1H), 1.89-1.71 (m, 3H).

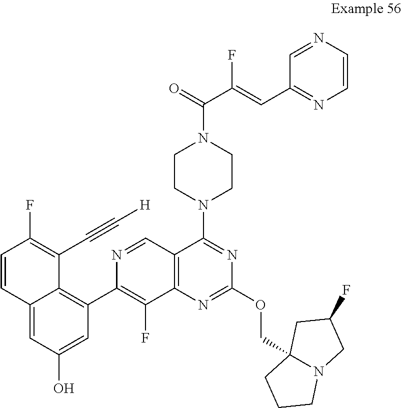

### (Z)-1-(4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyrazin-2-yl)prop-2-en-1-one

5-Ethynyl-6-fluoro-4-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol (5 mg, 0.008 mmol) and (Z)-2-fluoro-3-(pyrazin-2-yl)acrylic acid (5.9 mg, 0.035 mmol) were combined as solids and dissolved in DMF (300 μL). 1-Methylimidazole (5.6 μL, 0.07 mmol) was added followed by chloro-N,N,N′,N′-tetramethylformamidinium hexafluorophosphate (9.8 mg, 0.035 mmol). The reaction mixture was stirred for 5 min. A saturated aqueous solution of ammonium hydroxide (100 μL) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 85% 5:95 MeCN:H₂O with 10 mM AA/15% 95:5 MeCN:H₂O with 10 mM AA→100% 95:5 MeCN:H₂O with 10 mM AA; λ=220 nm) to provide the desired product (6.1 mg, 0.008 mmol, 95% yield) as a brown solid. LC/MS (ESI) m/z: [M+H]⁺ calcd for C₃₈H₃₃F₄N₈O₃ 725.3; found 725.3; ¹H NMR (500 MHz, DMSO-d₆) δ 10.18 (br s, 1H), 9.11 (s, 1H), 8.97 (s, 1H), 8.73 (d, J=1.4 Hz, 1H), 8.61 (d, J=2.4 Hz, 1H), 7.97 (dd, J=9.2, 6.0 Hz, 1H), 7.50-7.43 (m, 1H), 7.40 (d, J=2.4 Hz, 1H), 7.22-7.18 (m, 1H), 6.75 (d, J=38.6 Hz, 1H), 5.30 (d, J=54.6 Hz, 1H), 4.22-4.03 (m, 9H), 3.97 (s, 1H), 3.95-3.89 (m, 2H), 3.15-3.03 (m, 2H), 2.92-2.81 (m, 1H), 2.22-2.09 (m, 2H), 2.06-1.98 (m, 1H), 1.90-1.73 (m, 3H).

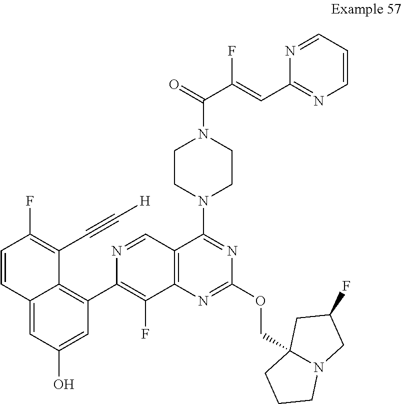

### (Z)-1-(4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyrimidin-2-yl)prop-2-en-1-one

5-Ethynyl-6-fluoro-4-(8-fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol (5 mg, 0.008 mmol)and (Z)-2-fluoro-3-(pyrimidin-2-yl)acrylic acid (5.9 mg, 0.035 mmol) were combined as solids and dissolved in DMF (300 μL). 1-Methylimidazole (5.6 μL, 0.07 mmol) was added followed by chloro-N,N,N′,N′-tetramethylformamidinium hexafluorophosphate (9.8 mg, 0.035 mmol). The reaction mixture was stirred for 5 min. A saturated aqueous solution of ammonium hydroxide (100 μL) was added, and the reaction mixture was stirred for an additional 5 min. The reaction mixture was directly purified by reverse phase HPLC (column: Xbridge C18, 19 mm×200 mm, 5 μm particles; flow rate: 20 mL/min; column temperature: 25° C.; gradient: 86% 5:95 MeCN:H₂O with 10 mM AA/14% 95:5 MeCN:H₂O with 10 mM AA→100% 95:5 MeCN:H₂O with 10 mM AA; λ=220 nm) to provide the desired product (5.2 mg, 0.007 mmol, 82% yield) as a brown solid. LC/MS (ESI) m/z: [M+H]⁺ calcd for C₃₈H₃₃F₄N₈O₃ 725.3; found 725.2; ¹H NMR (500 MHz, DMSO-d₆) δ 10.19 (br s, 1H), 9.10 (s, 1H), 8.89 (d, J=4.8 Hz, 2H), 7.97 (dd, J=9.2, 5.7 Hz, 1H), 7.50-7.43 (m, 2H), 7.39 (d, J=2.1 Hz, 1H), 7.19 (s, 1H), 6.63-6.63 (m, 1H), 6.63 (d, J=35.0 Hz, 1H), 5.28 (d, J=54.2 Hz, 1H), 4.20-3.99 (m, 9H), 3.97 (s, 1H), 3.13-3.05 (m, 2H), 3.04-2.99 (m, 1H), 2.87-2.76 (m, 1H), 2.15-2.09 (m, 1H), 2.08-2.05 (m, 1H), 2.04-1.98 (m, 1H), 1.87-1.73 (m, 3H).

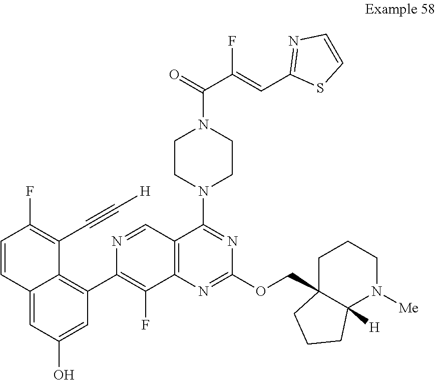 [SMILES: C#Cc1c(F)ccc2cc(O)cc(-c3ncc4c(N5CCN(C(=O)/C(F)=C/c6nccs6)CC5)nc(OC[C@]56CCC[C@H]5N(C)CCC6)nc4c3F)c12]

### (Z)-1-(4-(7-(8-ethynyl-7-fluoro-3-hydroxynaphthalen-1-yl)-8-fluoro-2-(((4aS, 7aR)-1-methyloctahydro-4aH-cyclopenta[b]pyridin-4a-yl)methoxy)pyrido[4,3-d]pyrimidin-4-yl)piperazin-1-yl)-2-fluoro-3-(thiazol-2-yl)prop-2-en-1-one

5-Ethynyl-6-fluoro-4-(8-fluoro-2-(((4aS,7aR)-1-methyloctahydro-4aH-cyclopenta[b]pyridin-4a-yl)methoxy)-4-(piperazin-1-yl)pyrido[4,3-d]pyrimidin-7-yl)naphthalen-2-ol (40 mg, 0.068 mmol) and (Z)-2-fluoro-3-(thiazol-2-yl)acrylic acid (47 mg, 0.27 mmol) were combined as solids and dissolved in DMF (1.4 mL). 1-Methylimidazole (44 μL, 0.55 mmol) was added followed by chloro-N,N,N′,N′-tetramethylformamidinium hexafluorophosphate (77 mg, 0.027 mmol). The reaction mixture was stirred for 5 min. A saturated aqueous solution of ammonium hydroxide (100 L) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 82% 5:95 MeCN:H₂O with 10 mM AA/18% 95:5 MeCN:H₂O with 10 mM AA→100% 95:5 MeCN:H₂O with 10 mM AA; λ=220 nm) to provide the desired product (29.4 mg, 0.04 mmol, 58% yield) as a brown solid. LC/MS (ESI) m/z: [M+H]⁺ calcd for C₃₉H₃₇F₃N₇O₃S 740.3; found 740.2; ¹H NMR (500 MHz, DMSO-d₆) δ 10.17 (br s, 1H), 9.11 (s, 1H), 8.03-7.94 (m, 3H), 7.46 (t, J=9.1 Hz, 1H), 7.40 (d, J=2.5 Hz, 1H), 7.20 (d, J=2.2 Hz, 1H), 7.11 (d, J=37.6 Hz, 1H), 4.54-4.44 (m, 1H), 4.22-4.07 (m, 5H), 4.02-3.82 (m, 5H), 3.27-3.10 (m, 2H), 2.41-2.10 (m, 3H), 1.89-1.82 (m, 1H), 1.77-1.19 (m, 10H).

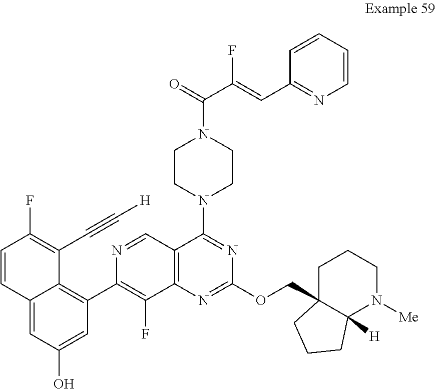 [SMILES: C#Cc1c(F)ccc2cc(O)cc(-c3ncc4c(N5CCN(C(=O)/C(F)=C/c6ccccn6)CC5)nc(OC[C@]56CCC[C@H]5N(C)CCC6)nc4c3F)c12]

### (Z)-1-(4-(7-(8-ethynyl-7-fluoro-3-hydroxynaphthalen-1-yl)-8-fluoro-2-(((4aS, 7aR)-1-methyloctahydro-4aH-cyclopenta[b]pyridin-4a-yl)methoxy)pyrido[4,3-d]pyrimidin-4-yl)piperazin-1-yl)-2-fluoro-3-(pyridin-2-yl)prop-2-en-1-one

5-Ethynyl-6-fluoro-4-(8-fluoro-2-(((4aS,7aR)-1-methyloctahydro-4aH-cyclopenta[b]pyridin-4a-yl)methoxy)-4-(piperazin-1-yl)pyrido[4,3-d]pyrimidin-7-yl)naphthalen-2-ol (40 mg, 0.068 mmol) and (Z)-2-fluoro-3-(pyridin-2-yl)acrylic acid (46 mg, 0.27 mmol) were combined as solids and dissolved in DMF (1.4 mL). 1-Methylimidazole (44 μL, 0.55 mmol) was added followed by chloro-N,N,N′,N′-tetramethylformamidinium hexafluorophosphate (77 mg, 0.027 mmol). The reaction mixture was stirred for 5 min. A saturated aqueous solution of ammonium hydroxide (100 L) was added, and the reaction mixture was stirred for an additional 5 min. The reaction mixture was directly purified by reverse phase HPLC (column: Xbridge C18, 19 mm×200 mm, 5 m particles; flow rate: 20 mL/min; column temperature: 25° C.; gradient: 82% 5:95 MeCN:H₂O with 10 mM AA/18% 95:5 MeCN:H₂O with 10 mM AA→100% 95:5 MeCN:H₂O with 10 mM AA; λ=220 nm) to provide the desired product (29.6 mg, 0.04 mmol, 58% yield) as a brown solid. LC/MS (ESI) m/z: [M+H]⁺ calcd for C₄₁H₃₉F₃N₇O₃ 734.3; found 734.3; ¹H NMR (500 MHz, DMSO-d₆) δ 10.17 (br s, 1H), 9.12 (s, 1H), 8.67-8.63 (m, 1H), 7.98 (dd, J=9.1, 6.0 Hz, 1H), 7.92-7.87 (m, 1H), 7.79 (d, J=7.9 Hz, 1H), 7.46 (t, J=9.0 Hz, 1H), 7.41-7.36 (m, 2H), 7.20 (d, J=2.4 Hz, 1H), 6.66 (d, J=38.8 Hz, 1H), 4.67-4.33 (m, 1H), 4.25-4.05 (m, 5H), 4.02-3.79 (m, 5H), 3.58-3.39 (m, 4H), 2.40-2.12 (m, 1H), 1.88-1.33 (m, 10H).

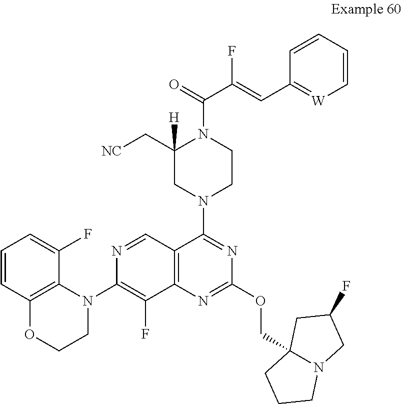

### 2-((S)-1-((Z)-2-fluoro-3-(pyridin-2-yl)acryloyl)-4-(8-fluoro-7-(5-fluoro-2,3-dihydro-4H-benzo[b][1,4]oxazin-4-yl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

2-((S)-4-(8-Fluoro-7-(5-fluoro-2,3-dihydro-4H-benzo[b][1,4]oxazin-4-yl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (5 mg, 8.6 μmol) and (Z)-2-fluoro-3-(pyridin-2-yl)acrylic acid (2.9 mg, 0.017 mmol) were combined as solids and dissolved in DMF (0.5 mL). 1-Methylimidazole (7 μL, 0.086 mmol) was added followed by chloro-N,N,N′,N′-tetramethylformamidinium hexafluorophosphate (4.8 mg, 0.017 mmol). The reaction mixture was stirred for 5 min. The reaction mixture was directly purified by reverse phase HPLC (column: Xbridge C18, 19 mm×200 mm, 5 μm particles; flow rate: 20 mL/min; column temperature: 25° C.; gradient: 71% 5:95 MeCN:H₂O with 10 mM AA/29%₀ 95:5 MeCN:H₂O with 10 mM AA→100% 95:5 MeCN:H₂O with 10 mM AA; λ=220 nm) to provide the desired product (1.8 mg, 2 μmol, 29% yield). LC/MS (ESI) m/z: [M+H]⁺ calcd for C₃₇H₃₆F₄N₉O₃ 730.3; found 730.2; ¹H NMR (500 MHUz, DMSO-d₆) δ 8.87 (s, 1H), 8.65 (br d, J=3.7 Hz, 1H), 7.92-7.86 (m, 1H), 7.78 (br d, J=8.4 Hz, 1H), 7.40-7.35 (m, 1H), 7.03-6.97 (m, 1H), 6.78 (br d, J=8.2 Hz, 1H), 6.76-6.70 (m, 1H), 6.64 (d, J=37.8 Hz, 1H), 5.27 (d, J=54.6 Hz, 1H), 4.93-4.77 (m, 1H), 4.47-4.40 (m, 1H), 4.38-4.31 (m, 1H), 4.30-4.23 (m, 1H), 4.22-4.16 (m, 1H), 4.15-3.87 (m, 6H), 3.79-3.54 (m, 4H), 3.13-3.01 (m, 3H), 2.88-2.79 (m, 1H), 2.13-1.98 (m, 3H), 1.87-1.76 (in, 3H).

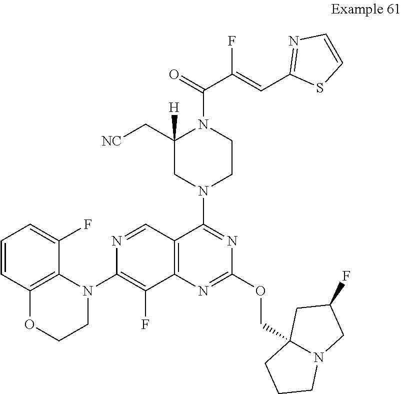 [SMILES: N#CC[C@H]1CN(c2nc(OC[C@@]34CCCN3C[C@H](F)C4)nc3c(F)c(N4CCOc5cccc(F)c54)ncc23)CCN1C(=O)/C(F)=C/c1nccs1]

### 2-((S)-1-((Z)-2-fluoro-3-(thiazol-2-yl)acryloyl)-4-(8-fluoro-7-(5-fluoro-2,3-dihydro-4H-benzo[b][1,4]oxazin-4-yl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

2-((S)-4-(8-Fluoro-7-(5-fluoro-2,3-dihydro-4H-benzo[b][1,4]oxazin-4-yl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (5 mg, 8.6 μmol) and (Z)-2-fluoro-3-(thiazol-2-yl)acrylic acid (3.0 mg, 0.017 mmol) were combined as solids and dissolved in DMF (0.5 mL). 1-Methylimidazole (7 μL, 0.086 mmol) was added followed by chloro-N,N,N′,N′-tetramethylformamidinium hexafluorophosphate (4.8 mg, 0.017 mmol). The reaction mixture was stirred for 5 min. The reaction mixture was directly purified by reverse phase HPLC (column: Xbridge C18, 19 mm×200 mm, 5 m particles; flow rate: 20 mL/min; column temperature: 25° C.; gradient: 70% 5:95 MeCN:H₂O with 10 mM AA/30% 95:5 MeCN:H₂O with 10 mM AA→100% 95:5 MeCN:H₂O with 10 mM AA; λ=220 nm) to provide the desired product (2.3 mg, 3 mol, 36% yield). LC/MS (ESI) m/z: [M+H]⁺ calcd for C₃₅H₃₄F₄N₉O₃S 736.2; found 736.2; ¹H NMR (500 MHz, DMSO-d₆) δ 8.87 (s, 1H), 8.04-7.99 (m, J=6.2 Hz, 2H), 7.08 (d, J=37.2 Hz, 1H), 7.03-6.97 (m, 1H), 6.80-6.71 (m, 2H), 5.27 (d, J=54.0 Hz, 1H), 4.92-4.82 (m, 1H), 4.43 (br d, J=14.0 Hz, 1H), 4.39-4.30 (m, 1H), 4.29-4.23 (m, 1H), 4.22-4.17 (m, 1H), 4.15-4.10 (m, 2H), 4.07-4.02 (m, 2H), 4.02-3.97 (m, 1H), 3.97-3.91 (m, 2H), 3.78-3.60 (m, 3H), 3.13-3.03 (m, 3H), 2.87-2.77 (m, 1H), 2.12-1.99 (m, 3H), 1.85-1.78 (m, 3H).

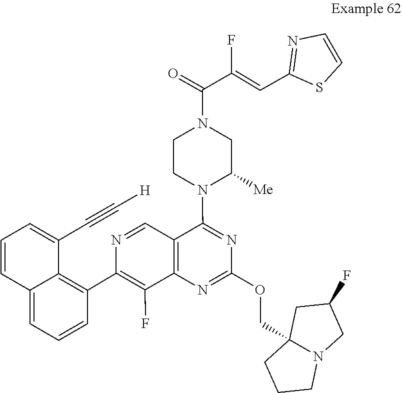 [SMILES: C#Cc1cccc2cccc(-c3ncc4c(N5CCN(C(=O)/C(F)=C/c6nccs6)C[C@@H]5C)nc(OC[C@@]56CCCN5C[C@H](F)C6)nc4c3F)c12]

### (Z)-1-((S)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(thiazol-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidine (25 mg, 0.045 mmol and (Z)-2-fluoro-3-(thiazol-2-yl)acrylic acid (23 mg, 0.14 mmol) were combined as solids and dissolved in DMF (500 μL). 1-Methylimidazole (22 μL, 0.27 mmol) was added followed by chloro-N,N,N′,N′-tetramethylformamidinium hexafluorophosphate (38 mg, 0.14 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 72% 5:95 MeCN:H₂O with 10 mM AA/28% 95:5 MeCN:H₂O with 10 mM AA→100% 95:5 MeCN:H₂O with 10 mM AA; λ=220 nm) to provide the desired product (17 mg, 0.024 mmol, 52% yield) as a brown solid. LC/MS (ESI) m/z: [M+H]⁺ calcd for C₃₈H₃₅F₃N₇O₂ S 710.3; found 710.1; ¹H NMR (500 MHz, DMSO-d₆) δ 9.10-9.01 (m, 1H), 8.19-8.10 (m, 2H), 8.07-7.95 (m, 2H), 7.77-7.66 (m, 2H), 7.65-7.55 (m, 2H), 7.09 (d, J=37.5 Hz, 1H), 5.27 (d, J=53.9 Hz, 1H), 5.09-4.84 (m, 1H), 4.52-4.31 (m, 1H), 4.25-3.91 (m, 5H), 3.85-3.67 (m, 3H), 3.12-2.99 (m, 2H), 2.88-2.77 (m, 2H), 2.15-1.97 (m, 3H), 1.87-1.75 (m, 3H), 1.52-1.39 (m, 3H).

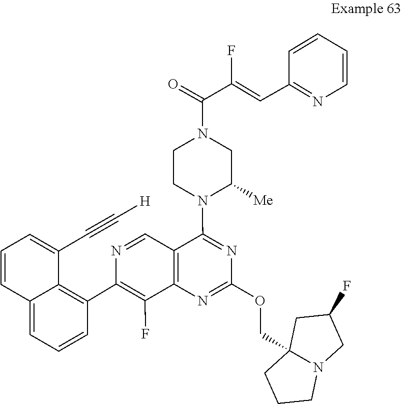 [SMILES: C#Cc1cccc2cccc(-c3ncc4c(N5CCN(C(=O)/C(F)=C/c6ccccn6)C[C@@H]5C)nc(OC[C@@]56CCCN5C[C@H](F)C6)nc4c3F)c12]

### (Z)-1-((S)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyridin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidine (25 mg, 0.045 mmol and (Z)-2-fluoro-3-(pyridin-2-yl)acrylic acid (22 mg, 0.14 mmol) were combined as solids and dissolved in DMF (500 μL). 1-methylimidazole (22 μL, 0.27 mmol) was added followed by chloro-N,N,N′,N′-tetramethylformamidinium hexafluorophosphate (38 mg, 0.14 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 73% 5:95 MeCN:H₂O with 10 mM AA/27% 95:5 MeCN:H₂O with 10 mM AA→100% 95:5 MeCN:H₂O with 10 mM AA; λ=220 nm) to provide the desired product (20.4 mg, 0.029 mmol, 64% yield) as a brown solid. LC/MS (ESI) m/z: [M+H]⁺ calcd for C₄₀H₃₇F₃N₇O₂ 704.3; found 704.3; ¹H NMR (500 MHz, DMSO-d₆) δ 9.13-8.95 (m, 1H), 8.70-8.58 (m, 1H), 8.24-8.09 (m, 2H), 7.96-7.85 (m, 1H), 7.83-7.76 (m, 1H), 7.74-7.68 (m, 2H), 7.64-7.54 (m, 2H), 7.38 (dd, J=7.7, 5.0 Hz, 1H), 6.63 (d, J=38.3 Hz, 1H), 5.27 (d, J=53.6 Hz, 1H), 5.09-4.83 (m, 1H), 4.53-4.31 (m, 1H), 4.28-3.93 (m, 5H), 3.83-3.64 (m, 3H), 3.12-3.01 (m, 3H), 2.87-2.77 (m, 1H), 2.19-1.94 (m, 3H), 1.87-1.73 (m, 3H), 1.53-1.38 (m, 3H).

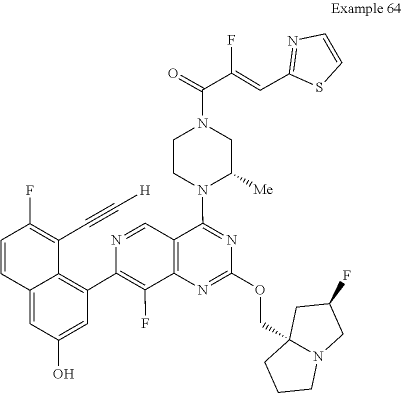 [SMILES: C#Cc1c(F)ccc2cc(O)cc(-c3ncc4c(N5CCN(C(=O)/C(F)=C/c6nccs6)C[C@@H]5C)nc(OC[C@@]56CCCN5C[C@H](F)C6)nc4c3F)c12]

### (Z)-1-((S)-4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(thiazol-2-yl)prop-2-en-1-one

5-Ethynyl-6-fluoro-4-(8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidin-7-yl)naphthalen-2-ol (25 mg, 0.042 mmol) and (Z)-2-fluoro-3-(thiazol-2-yl)acrylic acid (22 mg, 0.127 mmol) were combined as solids and dissolved in DMF (500 μL). 1-methylimidazole (20 μL, 0.26 mmol) was added followed by chloro-N,N,N′,N′-tetramethylformamidinium hexafluorophosphate (36 mg, 0.127 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product (17.4 mg, 0.023 mmol, 55% yield) as a brown solid. LC/MS (ESI) m/z: [M+H]⁺ calcd for C₃₈H₃₄F₄N₇O₃S 744.2; found 744.3; ¹H NMR (500 MHz, DMSO-d₆) δ 9.11-9.00 (m, 1H), 8.06-7.94 (m, 3H), 7.49-7.38 (m, 2H), 7.22-7.17 (m, 1H), 7.15-7.02 (m, 1H), 5.27 (d, J=54.1 Hz, 1H), 5.10-4.82 (m, 1H), 4.51-4.31 (m, 1H), 4.26-3.78 (m, 8H), 3.13-3.01 (m, 4H), 2.88-2.78 (m, 1H), 2.17-1.95 (m, 3H), 1.87-1.71 (m, 3H), 1.51-1.37 (m, 3H).

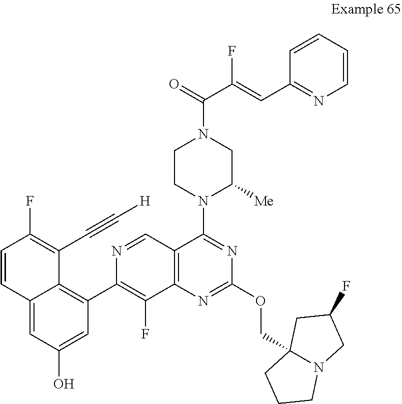 [SMILES: C#Cc1c(F)ccc2cc(O)cc(-c3ncc4c(N5CCN(C(=O)/C(F)=C/c6ccccn6)C[C@@H]5C)nc(OC[C@@]56CCCN5C[C@H](F)C6)nc4c3F)c12]

### (Z)-1-((S)-4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyridin-2-yl)prop-2-en-1-one

5-Ethynyl-6-fluoro-4-(8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidin-7-yl)naphthalen-2-ol (25 mg, 0.042 mmol) and (Z)-2-fluoro-3-(pyridin-2-yl)acrylic acid (21 mg, 0.127 mmol) were combined as solids and dissolved in DMF (500 μL). 1-methylimidazole (20 μL, 0.26 mmol) was added followed by chloro-N,N,N′,N′-tetramethylformamidinium hexafluorophosphate (36 mg, 0.127 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product (17.1 mg, 0.023 mmol, 54% yield) as a brown solid.LC/MS (ESI) m/z: [M+H]⁺ calcd for C₄₀H₃₆F₄N₇O₃ 738.3; found 738.3; ¹H NMR (500 MHz, DMSO-d₆) δ 9.10-8.99 (m, 1H), 8.68-8.61 (m, 1H), 8.03-7.95 (m, 1H), 7.92-7.85 (m, 1H), 7.85-7.76 (m, 1H), 7.50-7.44 (m, 1H), 7.42-7.33 (m, 2H), 7.24-7.14 (m, 1H), 6.63 (d, J=38.6 Hz, 1H), 5.27 (d, J=55.0 Hz, 1H), 5.08-4.81 (m, 1H), 4.52-4.32 (m, 1H), 4.27-3.76 (m, 6H), 3.14-2.98 (m, 4H), 2.86-2.78 (m, 1H), 2.15-1.95 (m, 3H), 1.87-1.73 (m, 3H), 1.50-1.38 (m, 3H).

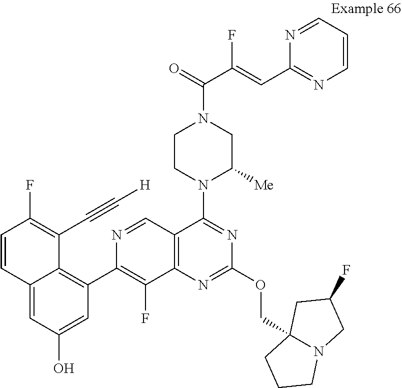 [SMILES: C#Cc1c(F)ccc2cc(O)cc(-c3ncc4c(N5CCN(C(=O)/C(F)=C/c6ncccn6)C[C@@H]5C)nc(OC[C@@]56CCCN5C[C@H](F)C6)nc4c3F)c12]

### (Z)-1-((S)-4-(7-(8-ethynyl-7-fluoro-3-hydroxynaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyrimidin-2-yl)prop-2-en-1-one

5-Ethynyl-6-fluoro-4-(8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidin-7-yl)naphthalen-2-ol (25 mg, 0.042 mmol) and (Z)-2-fluoro-3-(pyrimidin-2-yl)acrylic acid (21 mg, 0.127 mmol) were combined as solids and dissolved in DMF (500 μL). 1-methylimidazole (20 μL, 0.26 mmol) was added followed by chloro-N,N,N′,N′-tetramethylformamidinium hexafluorophosphate (36 mg, 0.127 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 78% 5:95 MeCN:H₂O with 10 mM AA/22% 95:5 MeCN:H₂O with 10 mM AA→100% 95:5 MeCN:H₂O with 10 mM AA; λ=220 nm) to provide the desired product (3.8 mg, 0.005 mmol, 12% yield) as a brown solid. LC/MS (ESI) m/z: [M+H]⁺ calcd for C₃₉H₃₅F₄N₈O₃ 739.3; found 739.3; ¹H NMR (500 MHz, DMSO-d₆) δ 9.12-9.00 (m, 1H), 8.89 (d, J=4.9 Hz, 2H), 8.03-7.93 (m, 1H), 7.51-7.38 (m, 3H), 7.25-7.16 (m, 1H), 6.60 (d, J=35.2 Hz, 1H), 5.27 (d, J=53.6 Hz, 1H), 5.11-4.86 (m, 1H), 4.51-4.31 (m, 1H), 4.19-4.01 (m, 3H), 4.00-3.87 (m, 2H), 3.85-3.73 (m, 1H), 3.14-3.00 (m, 4H), 2.86-2.79 (m, 1H), 2.15-1.95 (m, 3H), 1.87-1.72 (m, 3H), 1.52-1.35 (m, 3H).

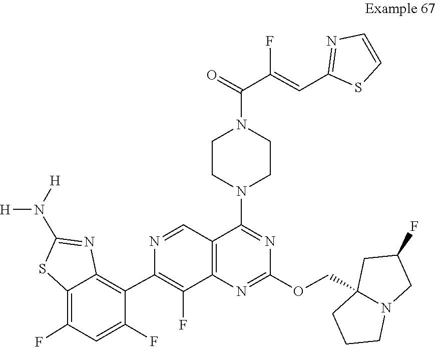 [SMILES: Nc1nc2c(-c3ncc4c(N5CCN(C(=O)/C(F)=C/c6nccs6)CC5)nc(OC[C@@]56CCCN5C[C@H](F)C6)nc4c3F)c(F)cc(F)c2s1]

### (Z)-1-(4-(7-(2-amino-5,7-difluorobenzo[d]thiazol-4-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(thiazol-2-yl)prop-2-en-1-one

tert-Butyl (5,7-difluoro-4-(8-fluoro-4-(4-((Z)-2-fluoro-3-(thiazol-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7-yl)benzo[d]thiazol-2-yl)carbamate (16 mg, 0.019 mmol) was dissolved in DCM (200 μL) and TFA (200 μL). The reaction mixture was stirred at room temperature for 4 h and was directly purified by prep HPLC (column: Xbridge C18, 19 mm×200 mm, 5 m particles; flow rate: 20 mL/min; column temperature: 25° C.; gradient: 78% 5:95 MeCN:H₂O with 10 mM AA/22% 95:5 MeCN:H₂O with 10 mM AA→100% 95:5 MeCN:H₂O with 10 mM AA; λ=220 nm) to provide the desired product (4.2 mg, 0.005 mmol, 29% yield). LC/MS (ESI) m/z: [M+H]⁺ calcd for C₃₂H₂₉F₅N₉O₂ S₂ 730.2; found 730.2; ¹H NMR (500 MHz, DMSO-d₆) δ 9.18 (s, 1H), 8.07 (br s, 2H), 8.03-7.97 (m, 2H), 7.15 (t, J=9.9 Hz, 1H), 7.10 (d, J=37.5 Hz, 1H), 5.28 (d, J=54.8 Hz, 1H), 4.22-4.03 (m, 6H), 4.01-3.81 (m, 4H), 3.13-3.01 (m, 3H), 2.86-2.79 (m, 1H), 2.15-1.95 (m, 3H), 1.87-1.73 (m, 3H).

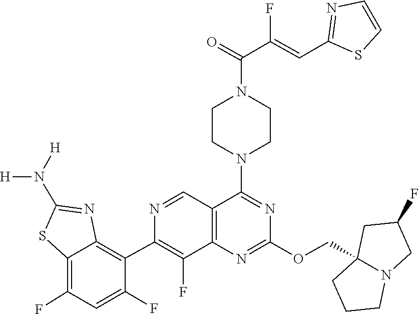 [SMILES: Nc1nc2c(-c3ncc4c(N5CCN(C(=O)/C(F)=C/c6nccs6)CC5)nc(OC[C@@]56CCCN5C[C@H](F)C6)nc4c3F)c(F)cc(F)c2s1]

### (Z)-1-(4-(7-(2-amino-7-fluorobenzo[d]thiazol-4-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(thiazol-2-yl)prop-2-en-1-one

tert-Butyl (7-fluoro-4-(8-fluoro-4-(4-((Z)-2-fluoro-3-(thiazol-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7-yl)benzo[d]thiazol-2-yl)carbamate (26 mg, 0.032 mmol) was dissolved in DCM (300 μL) and TFA (300 μL). The reaction mixture was stirred at room temperature for 4 h and was directly purified by prep HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product as the bis TFA salt (27 mg, 0.029 mmol, 90% yield). LC/MS (ESI) m/z: [M+H]⁺ calcd for C₃₂H₃₀F₄N₉O₂ S2 712.2; found 712.2.

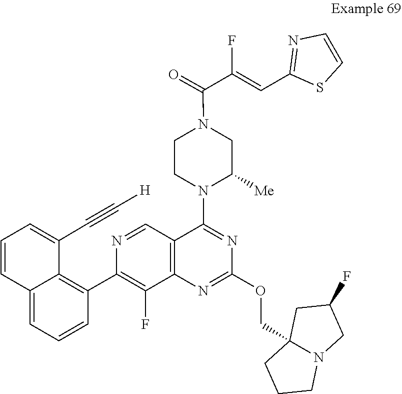 [SMILES: C#Cc1cccc2cccc(-c3ncc4c(N5CCN(C(=O)/C(F)=C/c6nccs6)C[C@@H]5C)nc(OC[C@@]56CCCN5C[C@H](F)C6)nc4c3F)c12]

### (Z)-1-((S)-4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(thiazol-2-yl)prop-2-en-1-one

7-(8-Ethynyl-7-fluoronaphthalen-1-yl)-8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidine (20 mg, 0.035 mmol) and (Z)-2-fluoro-3-(thiazol-2-yl)acrylic acid (18 mg, 0.11 mmol)were combined as solids and dissolved in DMF (700 μL). 1-methylimidazole (17 μL, 0.35 mmol) was added followed by chloro-N,N,N′,N′-tetramethylformamidinium hexafluorophosphate (29 mg, 0.11 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 70% 5:95 MeCN:H₂O with 10 mM AA/30% 95:5 MeCN:H₂O with 10 mM AA→100% 95:5 MeCN:H₂O with 10 mM AA; λ=220 nm) to provide the desired product (9 mg, 0.012 mmol, 35% yield) as a brown solid. LC/MS (ESI) m/z: [M+H]⁺ calcd for C₃₈H₃₄F₄N₇O₂ S 728.2; found 728.2; ¹H NMR (500 MHz, DMSO-d₆) δ 9.12-9.00 (m, 1H), 8.31-8.16 (m, 2H), 8.07-7.94 (m, 2H), 7.75-7.57 (m, 3H), 7.17-7.01 (m, 1H), 5.37-5.18 (m, 1H), 5.09-4.82 (m, 1H), 4.49-3.73 (m, 9H), 3.13-2.98 (m, 3H), 2.88-2.79 (m, 1H), 2.17-1.95 (m, 3H), 1.86-1.78 (m, 3H), 1.54-1.36 (m, 3H).

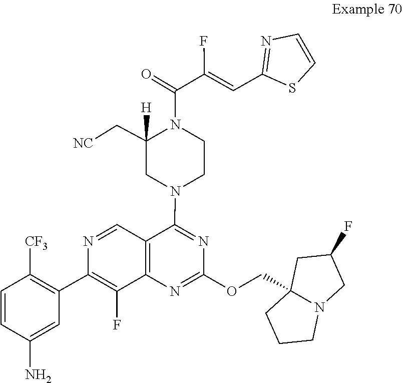 [SMILES: N#CC[C@H]1CN(c2nc(OC[C@@]34CCCN3C[C@H](F)C4)nc3c(F)c(-c4cc(N)ccc4C(F)(F)F)ncc23)CCN1C(=O)/C(F)=C/c1nccs1]

### (Z)-1-((S)-4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(thiazol-2-yl)prop-2-en-1-one

2-((S)-4-(7-(5-Amino-2-(trifluoromethyl)phenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (10 mg, 0.017 mmol) and (Z)-2-fluoro-3-(thiazol-2-yl)acrylic acid (3 mg, 0.017 mmol) were combined as solids and dissolved in DMF (400 μL). 1-methylimidazole (7 μL, 0.085 mmol) was added followed by chloro-N,N,N′,N′-tetramethylformamidinium hexafluorophosphate (5 mg, 0.017 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 78% 5:95 MeCN:H₂O with 10 mM AA/22% 95:5 MeCN:H₂O with 10 mM AA→100% 95:5 MeCN:H₂O with 10 mM AA; λ=220 nm) to provide the desired product (12 mg, 0.016 mmol, 94% yield) as an off-white solid. LCMS (ESI) m/z: [M+H]⁺ calcd for C₃₄H₃₂F₆N₉O₂ S 744.2; found 744.4; ¹H NMR (500 MHz, DMSO-d₆) δ 9.14 (s, 1H), 8.07-7.97 (m, 2H), 7.48 (d, J=8.6 Hz, 1H), 7.10 (d, J=38.1 Hz, 1H), 6.76 (br d, J=7.9 Hz, 1H), 6.58-6.54 (m, 1H), 6.00 (br s, 2H), 5.28 (d, J=54.5 Hz, 1H), 4.94-4.84 (m, 1H), 4.55-4.49 (m, 1H), 4.46-4.40 (m, 1H), 4.16 (br d, J=10.2 Hz, 1H), 4.08 (br d, J=10.2 Hz, 1H), 3.84-3.79 (m, 4H), 3.13-3.01 (m, 5H), 2.85-2.80 (m, 1H), 2.15-1.97 (m, 3H), 1.87-1.75 (m, 3H).

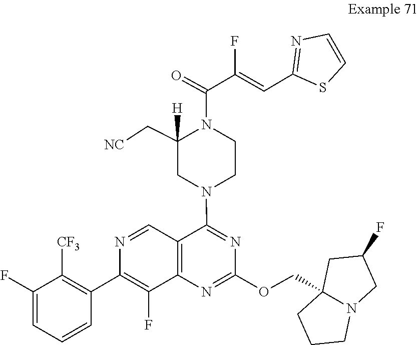 [SMILES: N#CC[C@H]1CN(c2nc(OC[C@@]34CCCN3C[C@H](F)C4)nc3c(F)c(-c4cccc(F)c4C(F)(F)F)ncc23)CCN1C(=O)/C(F)=C/c1nccs1]

### 2-((S)-1-((Z)-2-fluoro-3-(thiazol-2-yl)acryloyl)-4-(8-fluoro-7-(3-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile

2-((S)-4-(8-Fluoro-7-(3-fluoro-2-(trifluoromethyl)phenyl)-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (10 mg, 0.017 mmol) and (Z)-2-fluoro-3-(thiazol-2-yl)acrylic acid (9 mg, 0.051 mmol) were combined as solids and dissolved in DMF (400 μL). 1-methylimidazole (7 μL, 0.085 mmol) was added followed by chloro-N,N,N′,N′-tetramethylformamidinium hexafluorophosphate (14 mg, 0.051 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 70% 5:95 MeCN:H₂O with 10 mM AA/30% 95:5 MeCN:H₂O with 10 mM AA→100% 95:5 MeCN:H₂O with 10 mM AA; λ=220 nm) to provide the desired product (8.9 mg, 0.012 mmol, 70% yield) as an off-white solid. LCMS (ESI) m/z: [M+H]⁺ calcd for C₃₄H₃₀F₇N₈O₂ S 744.2; found 744.4; ¹H NMR (500 MHz, DMSO-d₆) δ 9.19 (s, 1H), 8.07-7.99 (m, 2H), 7.93-7.87 (m, 1H), 7.73-7.67 (m, 1H), 7.43 (d, J=7.6 Hz, 1H), 7.10 (d, J=37.4 Hz, 1H), 5.28 (d, J=53.4 Hz, 1H), 4.93-4.83 (m, 1H), 4.56-4.50 (m, 1H), 4.48-4.40 (m, 1H), 4.18-4.14 (m, 1H), 4.08 (d, J=10.2 Hz, 1H), 3.87-3.61 (m, 4H), 3.12-3.01 (m, 5H), 2.87-2.79 (m, 1H), 2.15-1.96 (m, 3H), 1.87-1.75 (m, 3H).

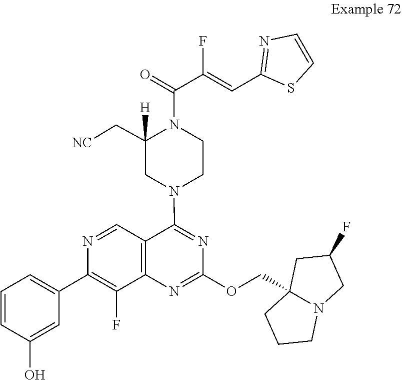 [SMILES: N#CC[C@H]1CN(c2nc(OC[C@@]34CCCN3C[C@H](F)C4)nc3c(F)c(-c4cccc(O)c4)ncc23)CCN1C(=O)/C(F)=C/c1nccs1]

### 2-((S)-4-(8-fluoro-2-(((2R, 7aS)-2-fluorotetrahydro-1H-pyrrolizin-7a(5H)-yl)methoxy)-7-(3-hydroxyphenyl)pyrido[4,3-d]pyrimidin-4-yl)-1-((Z)-2-fluoro-3-(thiazol-2-yl)acryloyl)piperazin-2-yl)acetonitrile

2-((S)-4-(8-Fluoro-2-(((2R, 7aS)-2-fluorotetrahydro-1H-pyrrolizin-7a(5H)-yl)methoxy)-7-(3-hydroxyphenyl)pyrido[4,3-d]pyrimidin-4-yl)piperazin-2-yl)acetonitrile (10 mg, 0.019 mmol and (Z)-2-fluoro-3-(thiazol-2-yl)acrylic acid (10 mg, 0.058 mmol) were combined as solids and dissolved in DMF (400 μL). 1-methylimidazole (7.6 μL, 0.096 mmol) was added followed by chloro-N,N,N′,N′-tetramethylformamidinium hexafluorophosphate (16 mg, 0.058 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 83% 5:95 MeCN:H₂O with 10 mM AA/17% 95:5 MeCN:H₂O with 10 mM AA→100% 95:5 MeCN:H₂O with 10 mM AA; λ=220 nm) to provide the desired product (4.2 mg, 6.12 μmol, 32% yield) as an off-white solid. LC/MS (ESI) m/z: [M+H]⁺ calcd for C₃₃H₃₂F₃N₈O₃S 677.2; found 677.2; ¹H NMR (500 MHz, DMSO-d₆) δ 9.20 (s, 1H), 8.10-7.97 (m, 2H), 7.51-7.45 (m, 2H), 7.36 (t, J=8.1 Hz, 1H), 7.11 (d, J=37.9 Hz, 1H), 6.95-6.88 (m, 1H), 5.29 (d, J=54.4 Hz, 1H), 4.95-4.85 (m, 1H), 4.55-4.48 (m, 1H), 4.47-4.40 (m, 1H), 4.18 (d, J=10.4 Hz, 1H), 4.10 (d, J=10.3 Hz, 1H), 3.87-3.67 (m, 4H), 3.13-3.02 (m, 5H), 2.90-2.81 (m, 1H), 2.18-1.98 (m, 3H), 1.88-1.77 (m, 3H).

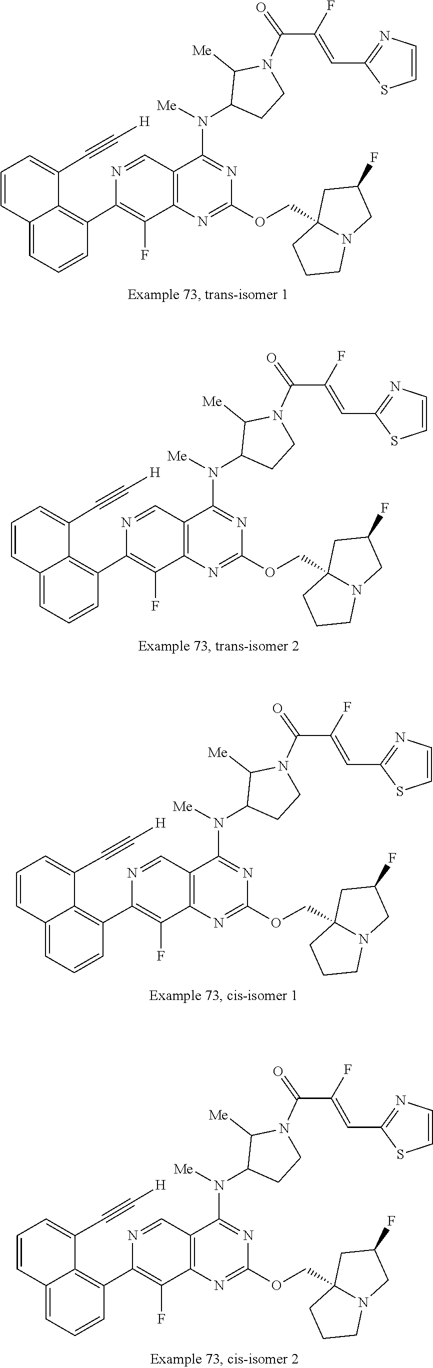 [SMILES: C#Cc1cccc2cccc(-c3ncc4c(N(C)C5CCN(C(=O)/C(F)=C/c6nccs6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6nccs6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6nccs6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6nccs6)C5C)nc(OC[C@@]56CCCN5C[C@H](F)C6)nc4c3F)c12]

### (Z)-1-(3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidin-1-yl)-2-fluoro-3-(thiazol-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-N-methyl-N-(2-methylpyrrolidin-3-yl)pyrido[4,3-d]pyrimidin-4-amine (95 mg, 0.167 mmol) and (Z)-2-fluoro-3-(thiazol-2-yl)acrylic acid (58 mg, 0.334 mmol) were combined as solids and dissolved in MeCN (800 μL). 1-methylimidazole (40 μL, 0.5 mmol) was added followed by chloro-N,N,N′,N′-tetramethylformamidinium hexafluorophosphate (94 mg, 0.334 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 83% 5:95 MeCN:H₂O with 10 mM AA/17% 95:5 MeCN:H₂O with 10 mM AA→100% 95:5 MeCN:H₂O with 10 mM AA; λ=220 nm) to provide two isolates: the first (70.3 mg, 0.097 mmol, 58% yield) as a mixture of cis isomers, and the second (7.3 mg, 0.010 mmol, 6.0% yield) as a mixture of trans isomers.

The mixture of trans isomers was further purified by preparative SFC (column: Chiralcel OD-H, 30 mm×250 mm, 5 μm particles; flow rate: 100 mL/min; column temperature: 40° C.; isocratic: 60% CO₂:40% MeOH with 20 mM ammonia) to provide both title trans isomers.

Trans isomer 1 (first eluting): 1.8 mg, 0.002 mmol, 45% recovery; LC/MS (ESI) m/z: [M+H]⁺ calcd for C₃₉H₃₇F₃N₇O₂ S 724.3; found 724.3; ¹H NMR (500 MHz, DMSO-d₆) δ 9.28-9.14 (m, 1H), 8.19-8.10 (m, 2H), 8.04-7.96 (m, 2H), 7.75-7.67 (m, 2H), 7.63-7.53 (m, 2H), 7.19 (d, J=36.9 Hz, 1H), 5.34 (d, J=54.0 Hz, 1H), 5.18-5.03 (m, 1H), 4.43-3.92 (m, 3H), 3.90 (s, 1H), 3.80-3.72 (m, 1H), 3.45 (s, 3H), 3.19-3.01 (m, 4H), 2.87-2.78 (m, 1H), 2.46-2.25 (m, 2H), 2.16-1.98 (m, 3H), 1.91-1.74 (m, 3H), 1.47-1.35 (m, 3H).

Trans isomer 2 (second eluting): 1.2 mg, 0.0015 mmol, 32% recovery; LC/MS (ESI) m/z: [M+H]⁺ calcd for C₃₉H₃₇F₃N₇O₂ S 724.3; found 724.3; ¹H NMR (500 MHz, DMSO-d₆) δ 9.25-9.17 (m, 1H), 8.18-8.10 (m, 2H), 8.04-7.98 (m, 2H), 7.74-7.67 (m, 2H), 7.62-7.54 (m, 2H), 7.19 (d, J=36.8 Hz, 1H), 5.26 (d, J=54.5 Hz, 1H), 5.19-5.04 (m, 1H), 4.41-4.03 (m, 3H), 3.90 (s, 1H), 3.79-3.72 (m, 1H), 3.45 (s, 3H), 3.19-3.01 (m, 4H), 2.86-2.78 (m, 1H), 2.46-2.24 (m, 2H), 2.18-1.97 (m, 3H), 1.90-1.75 (m, 3H), 1.48-1.36 (m, 3H).

The mixture of cis isomers was further purified by preparative SFC (column: Chiralcel OD-H, 30 mm×250 mm, 5 m particles; flow rate: 100 mL/min; column temperature: 40° C.; isocratic: 80% CO₂:20% MeOH with 20 mM ammonia) to provide bot title cis isomers.

Cis isomer 1 (first eluting): 19.8 mg, 0.027 mmol, 56% recovery; LC/MS (ESI) m/z: [M+H]⁺ calcd for C₃₉H₃₇F₃N₇O₂ S 724.3; found 724.3; ¹H NMR (500 MHz, DMSO-d₆) δ 9.26 (s, 1H), 8.18-8.12 (m, 2H), 8.04-8.00 (m, 2H), 7.75-7.65 (m, 2H), 7.65-7.55 (m, 2H), 7.22 (d, J=36.8 Hz, 1H), 5.39-4.79 (m, 3H), 4.69-4.55 (m, 2H), 4.23-4.03 (m, 2H), 3.98-3.83 (m, 2H), 3.62 (br s, 3H), 3.12-2.96 (m, 3H), 2.67-2.55 (m, 1H), 2.42-2.33 (m, 1H), 2.18-1.91 (m, 3H), 1.85-1.68 (m, 3H), 1.50-1.35 (m, 3H).

Cis isomer 2 (second eluting): 10.9 mg, 0.015 mmol, 31% recovery.; LC/MS (ESI) m/z: [M+H]⁺ calcd for C₃₉H₃₇F₃N₇O₂ S 724.3; found 724.3; ¹H NMR (500 MHz, DMSO-d₆) δ 9.26 (s, 1H), 8.17-8.12 (m, 2H), 8.04-7.98 (m, 2H), 7.74-7.67 (m, 2H), 7.63-7.55 (m, 2H), 7.22 (d, J=36.6 Hz, 1H), 5.35-4.79 (m, 3H), 4.70-4.45 (m, 2H), 4.20-4.01 (m, 2H), 4.01-3.87 (m, 2H), 3.62 (br s, 3H), 3.15-2.72 (m, 3H), 2.66-2.54 (m, 1H), 2.42-2.33 (m, 1H), 2.18-1.92 (m, 3H), 1.8501.68 (m, 3H).

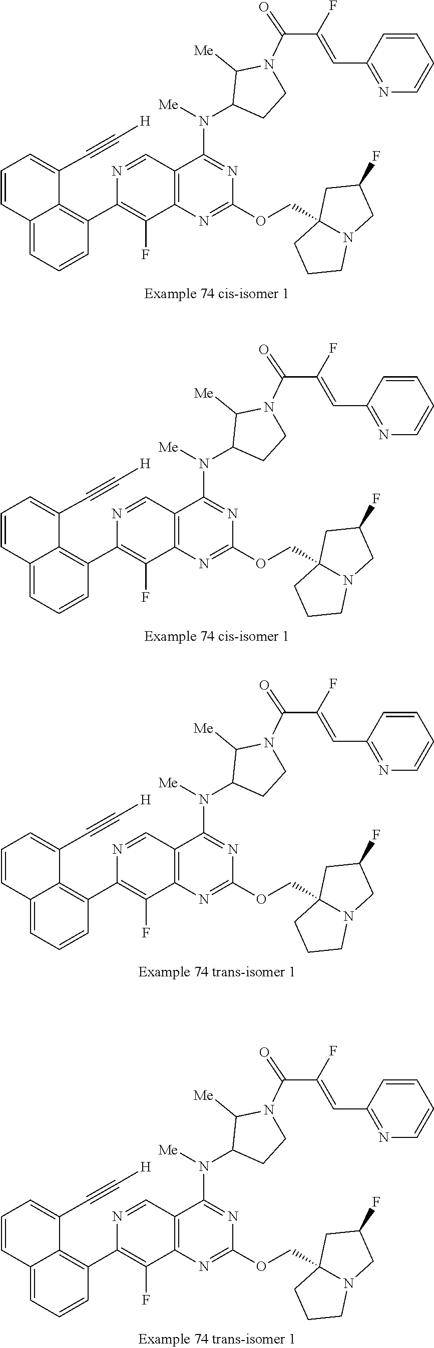 [SMILES: C#Cc1cccc2cccc(-c3ncc4c(N(C)C5CCN(C(=O)/C(F)=C/c6ccccn6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6ccccn6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6ccccn6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6ccccn6)C5C)nc(OC[C@@]56CCCN5C[C@H](F)C6)nc4c3F)c12]

### (Z)-1-(3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-2-methylpyrrolidin-1-yl)-2-fluoro-3-(pyridin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-N-methyl-N-(2-methylpyrrolidin-3-yl)pyrido[4,3-d]pyrimidin-4-amine (95 mg, 0.167 mmol) and (Z)-2-fluoro-3-(pyridin-2-yl)acrylic acid (56 mg, 0.334 mmol) were combined as solids and dissolved in MeCN (800 μL). 1-methylimidazole (40 μL, 0.5 mmol) was added followed by chloro-N,N,N′,N′-tetramethylformamidinium hexafluorophosphate (94 mg, 0.334 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide two isolates: the first (98.6 mg, 0.137 mmol, 82% yield) as a mixture of cis isomers, and the second (12.1 mg, 0.017 mmol, 10% yield) as a mixture of trans isomers.

The mixture of cis isomers was further purified by preparative SFC (column: Chiralpak IH, 21 mm×250 mm, 5 μm particles; flow rate: 75 mL/min; column temperature: 40° C.; isocratic: 80% CO₂:20% MeOH with 20 mM ammonia) to provide both title cis isomers.

Cis isomer 1 (first eluting): 35.3 mg, 0.048 mmol, 71% recovery; LC/MS (ESI) m/z: [M+H]⁺ calcd for C₄₁H₃₉F₃N₇O₂ 718.3; found 718.3; ¹H NMR (500 MHz, DMSO-d₆) δ 9.27-9.22 (m, 1H), 8.64 (br d, J=4.7 Hz, 1H), 8.14 (dd, J=10.9, 8.8 Hz, 2H), 7.93-7.84 (m, 1H), 7.77 (br d, J=7.2 Hz, 1H), 7.74-7.67 (m, 2H), 7.62-7.54 (m, 2H), 7.41-7.35 (m, 1H), 6.81 (d, J=37.8 Hz, 1H), 5.47-4.77 (m, 3H), 4.26-3.94 (m, 3H), 3.91-3.73 (m, 2H), 3.61 (br d, J=4.6 Hz, 3H), 3.20-2.79 (m, 4H), 2.43-2.31 (m, 1H), 2.17-1.59 (m, 7H), 1.22-1.07 (m, 3H).

Cis isomer 2 (second eluting): 38.3 mg, 0.051 mmol, 75% recovery; LC/MS (ESI) m/z: [M+H]⁺ calcd for C₄₁H₃₉F₃N₇O₂ 718.3; found 718.3; ¹H NMR (500 MHz, DMSO-d₆) δ 9.25-9.17 (m, 1H), 8.18-8.10 (m, 2H), 8.04-7.98 (m, 2H), 7.74-7.67 (m, 2H), 7.62-7.54 (m, 2H), 7.19 (d, J=36.8 Hz, 1H), 5.26 (d, J=54.5 Hz, 1H), 5.19-5.04 (m, 1H), 4.41-4.03 (m, 3H), 3.90 (s, 1H), 3.79-3.72 (m, 1H), 3.45 (s, 3H), 3.19-3.01 (m, 4H), 2.86-2.78 (m, 1H), 2.46-2.24 (m, 2H), 2.18-1.97 (m, 3H), 1.90-1.75 (m, 3H), 1.48-1.36 (m, 3H).

The mixture of trans isomers was further purified by preparative SFC (column: Chiralcel OD-H, 30 mm×250 mm, 5 m particles; flow rate: 100 mL/min; column temperature: 40° C.; isocratic: 80% CO₂:20% MeOH with 20 mM ammonia) to provide both title trans isomers.

Trans isomer 1 (first eluting): 3.9 mg, 0.0056 mmol, 63% recovery; LC/MS (ESI) m/z: [M+H]⁺ calcd for C₄₁H₃₉F₃N₇O₂ 718.3; found 718.3; ¹H NMR (500 MHz, DMSO-d₆) δ 9.32-9.15 (m, 1H), 8.71-8.58 (m, 1H), 8.15 (dd, J=11.3, 8.4 Hz, 2H), 7.94-7.85 (m, 1H), 7.83-7.77 (m, 1H), 7.75-7.67 (m, 2H), 7.64-7.53 (m, 2H), 7.44-7.34 (m, 1H), 6.80 (d, J=37.9 Hz, 1H), 5.47-5.24 (m, 1H), 5.21-5.07 (m, 1H), 4.43-4.00 (m, 4H), 3.97-3.83 (m, 2H), 3.83-3.73 (m, 1H), 3.46 (s, 3H), 3.20-2.83 (m, 3H), 2.43-2.03 (m, 5H), 1.94-1.81 (m, 3H), 1.50-1.42 (m, 3H).

Trans isomer 2 (second eluting): 3.3 mg, 0.0049 mmol, 54% recovery; LC/MS (ESI) m/z: [M+H]⁺ calcd for C₄₁H₃₉F₃N₇O₂ 718.3; found 718.3; ¹H NMR (500 MHz, DMSO-d₆) δ 9.30-9.18 (m, 1H), 8.65 (br d, J=4.1 Hz, 1H), 8.19-8.10 (m, 2H), 7.94-7.86 (m, 1H), 7.83-7.78 (m, 1H), 7.76-7.68 (m, 2H), 7.63-7.54 (m, 2H), 7.40-7.31 (m, 1H), 6.80 (d, J=37.5 Hz, 1H), 5.35 (d, J=50.7 Hz, 1H), 5.21-5.05 (m, 1H), 4.42-4.01 (m, 3H), 3.97-3.85 (m, 3H), 3.46 (s, 3H), 3.24-3.02 (m, 2H), 3.01-2.84 (m, 1H), 2.16 (br s, 6H), 2.00-1.84 (m, 3H), 1.49-1.42 (m, 3H).

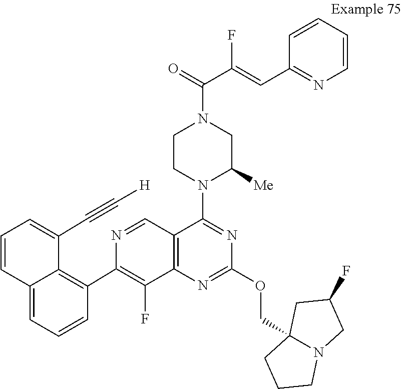 [SMILES: C#Cc1cccc2cccc(-c3ncc4c(N5CCN(C(=O)/C(F)=C/c6ccccn6)C[C@H]5C)nc(OC[C@@]56CCCN5C[C@H](F)C6)nc4c3F)c12]

### (Z)-1-((3R)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyridin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-((R)-2-methylpiperazin-1-yl)pyrido[4,3-d]pyrimidine (7.5 mg, 0.014 mmol and (Z)-2-fluoro-3-(pyridin-2-yl)acrylic acid (6.8 mg, 0.041 mmol) were combined as solids and dissolved in DMF (500 μL). 1-methylimidazole (6.5 μL, 0.081 mmol) was added followed by chloro-N,N,N′,N′-tetramethylformamidinium hexafluorophosphate (11 mg, 0.041 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (2.6 mg, 0.004 mmol, 27% yield) as a brown solid. LCMS (ESI) m/z: [M+H]⁺ calcd for C₄₀H₃₇F₃N₇O₂ 704.3; found 704.4; ¹H NMR (500 MHz, DMSO-d₆) δ 9.09-9.01 (m, 1H), 8.68-8.62 (m, 1H), 8.15 (dd, J=11.8, 8.5 Hz, 2H), 7.92-7.86 (m, 1H), 7.79 (d, J=7.9 Hz, 1H), 7.75-7.68 (m, 2H), 7.63-7.54 (m, 2H), 7.38 (dd, J=6.8, 4.9 Hz, 1H), 6.63 (d, J=38.6 Hz, 1H), 5.27 (d, J=53.9 Hz, 1H), 5.09-4.83 (m, 1H), 4.50-4.31 (m, 1H), 4.26-4.03 (m, 4H), 3.95-3.80 (m, 2H), 3.74-3.69 (m, 1H), 3.10-3.02 (m, 4H), 2.87-2.79 (m, 1H), 2.16-1.96 (m, 3H), 1.84-1.73 (m, 3H), 1.51-1.38 (m, 3H).

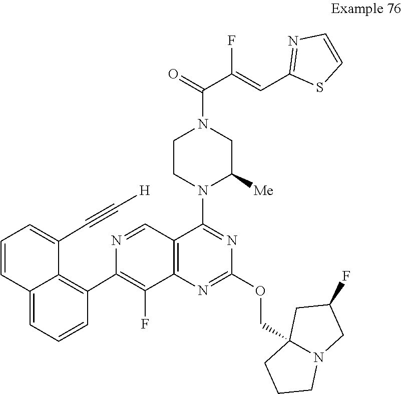 [SMILES: C#Cc1cccc2cccc(-c3ncc4c(N5CCN(C(=O)/C(F)=C/c6nccs6)C[C@H]5C)nc(OC[C@@]56CCCN5C[C@H](F)C6)nc4c3F)c12]

### (Z)-1-((3R)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(thiazol-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-((R)-2-methylpiperazin-1-yl)pyrido[4,3-d]pyrimidine (7.5 mg, 0.014 mmol and (Z)-2-fluoro-3-(thiazol-2-yl)acrylic acid (7.0 mg, 0.041 mmol) were combined as solids and dissolved in DMF (500 μL). 1-methylimidazole (6.5 μL, 0.081 mmol) was added followed by chloro-N,N,N′,N′-tetramethylformamidinium hexafluorophosphate (11 mg, 0.041 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (3.2 mg, 0.005 mmol, 33% yield) as a brown solid. LCMS (ESI) m/z: [M+H]⁺ calcd for C₃₈H₃₅F₃N₇O₂ S 710.3; found 710.3; ¹H NMR (500 MHz, DMSO-d₆) δ 9.10-8.99 (m, 1H), 8.23-8.11 (m, 2H), 8.05-7.95 (m, 2H), 7.77-7.67 (m, 2H), 7.64-7.54 (m, 2H), 7.16-7.03 (m, 1H), 5.39-5.19 (m, 1H), 5.08-4.81 (m, 1H), 4.50-4.32 (m, 1H), 4.26-4.01 (m, 4H), 3.99-3.91 (m, 1H), 3.85-3.78 (m, 1H), 3.73-3.67 (m, 1H), 3.12-3.00 (m, 4H), 2.87-2.79 (m, 1H), 2.13-1.97 (m, 3H), 1.87-1.75 (m, 3H), 1.51-1.39 (m, 3H).

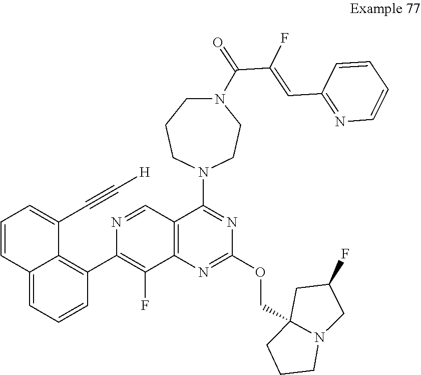

### (Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-1,4-diazepan-1-yl)-2-fluoro-3-(pyridin-2-yl)prop-2-en-1-one

4-(1,4-Diazepan-1-yl)-7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidine (6 mg, 0.011 mmol) and (Z)-2-fluoro-3-(pyridin-2-yl)acrylic acid (5.4 mg, 0.032 mmol) were combined as solids and dissolved in DMF (400 μL). 1-methylimidazole (5.2 μL, 0.065 mmol) was added followed by chloro-N,N,N′,N′-tetramethylformamidinium hexafluorophosphate (9.1 mg, 0.032 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (4.1 mg, 0.006 mmol, 54% yield) as a brown solid. LCMS (ESI) m/z: [M+H]⁺ calcd for C₄₀H₃₇F₃N₇O₂ 704.3; found 704.3; ¹H NMR (500 MHz, DMSO-d₆) δ 9.21-9.06 (m, 1H), 8.66-8.54 (m, 1H), 8.14 (t, J=9.2 Hz, 2H), 7.90-7.80 (m, 1H), 7.76-7.67 (m, 3H), 7.66-7.52 (m, 2H), 7.35 (dd, J=7.3, 4.8 Hz, 1H), 6.51-6.27 (m, 1H), 5.35-5.07 (m, 1H), 4.43-3.99 (m, 7H), 3.93-3.85 (m, 1H), 3.77-3.61 (m, 2H), 3.11-2.96 (m, 4H), 2.87-2.75 (m, 1H), 2.31-1.93 (m, 4H), 1.88-1.61 (m, 4H).

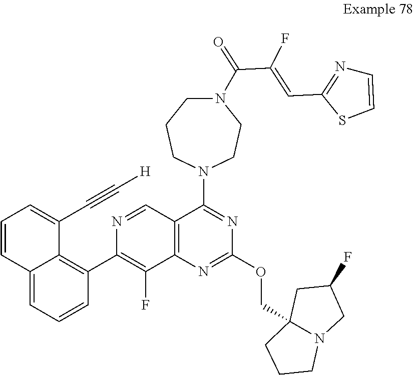

### (Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-1,4-diazepan-1-yl)-2-fluoro-3-(thiazol-2-yl)prop-2-en-1-one

4-(1,4-Diazepan-1-yl)-7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidine (6 mg, 0.011 mmol) and (Z)-2-fluoro-3-(thiazol-2-yl)acrylic acid (5.6 mg, 0.032 mmol) were combined as solids and dissolved in DMF (400 μL). 1-methylimidazole (5.2 μL, 0.065 mmol) was added followed by chloro-N,N,N′,N′-tetramethylformamidinium hexafluorophosphate (9.1 mg, 0.032 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (2.2 mg, 0.003 mmol, 29% yield) as a brown solid. LCMS (ESI) m/z: [M+H]⁺ calcd for C₃₈H₃₅F₃N₇O₂ S 710.3; found 710.3; ¹H NMR (500 MHz, DMSO-d₆) δ 9.26-9.01 (m, 1H), 8.19 (br d, J=1.8 Hz, 2H), 8.02-7.94 (m, 2H), 7.75-7.66 (m, 2H), 7.65-7.53 (m, 2H), 7.00-6.77 (m, 1H), 5.36-5.12 (m, 1H), 4.41-3.95 (m, 8H), 3.89-3.83 (m, 1H), 3.75-3.61 (m, 1H), 3.11-2.94 (m, 4H), 2.84-2.77 (m, 1H), 2.29-1.91 (m, 4H), 1.87-1.62 (m, 4H).

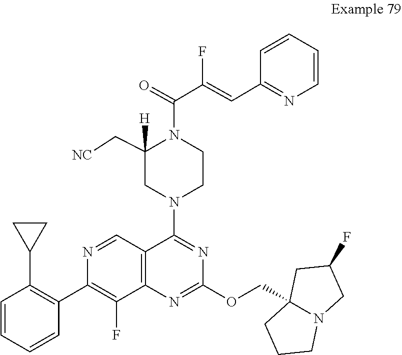 [SMILES: N#CC[C@H]1CN(c2nc(OC[C@@]34CCCN3C[C@H](F)C4)nc3c(F)c(-c4ccccc4C4CC4)ncc23)CCN1C(=O)/C(F)=C/c1ccccn1]

### 2-((2S)-4-(7-(2-cyclopropylphenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(pyridin-2-yl)acryloyl)piperazin-2-yl)acetonitrile

2-((2S)-4-(7-(2-Cyclopropylphenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (10 mg, 0.018 mmol) and (Z)-2-fluoro-3-(pyridin-2-yl)acrylic acid (9.2 mg, 0.055 mmol) were combined as solids and dissolved in DMF (600 μL). 1-methylimidazole (8.8 μL, 0.11 mmol) was added followed by chloro-N,N,N′,N′-tetramethylformamidinium hexafluorophosphate (15 mg, 0.055 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide the desired product (9.5 mg, 0.014 mmol, 75% yield) as an off-white solid. LCMS (ESI) m/z: [M+H]⁺ calcd for C₃₈H₃₈F₃N₈O₂ 695.3; found 695.3; ¹H NMR (500 MHz, DMSO-d₆) δ 9.22 (s, 1H), 8.69-8.62 (m, 1H), 7.95-7.86 (m, 1H), 7.80 (d, J=8.2 Hz, 1H), 7.44-7.36 (m, 2H), 7.36-7.26 (m, 2H), 7.04 (d, J=7.6 Hz, 1H), 6.66 (d, J=39.0 Hz, 1H), 5.28 (d, J=54.8 Hz, 1H), 4.99-4.85 (m, 1H), 4.56-4.41 (m, 2H), 4.16 (d, J=10.4 Hz, 1H), 4.08 (d, J=10.4 Hz, 1H), 4.24-4.01 (m, 1H), 3.88-3.70 (m, 2H), 3.19-3.16 (m, 1H), 3.14-3.01 (m, 5H), 2.87-2.80 (m, 1H), 2.15-1.97 (m, 3H), 1.88-1.72 (m, 4H), 0.77 (br d, J=8.3 Hz, 2H), 0.63 (br d, J=4.2 Hz, 2H).

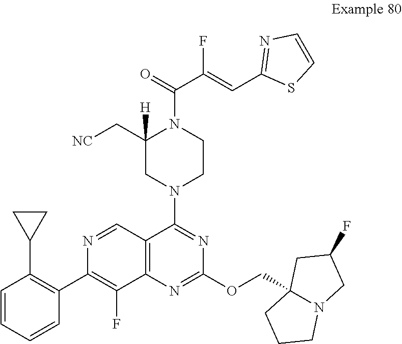 [SMILES: N#CC[C@H]1CN(c2nc(OC[C@@]34CCCN3C[C@H](F)C4)nc3c(F)c(-c4ccccc4C4CC4)ncc23)CCN1C(=O)/C(F)=C/c1nccs1]

### 2-((2S)-4-(7-(2-cyclopropylphenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(thiazol-2-yl)acryloyl)piperazin-2-yl)acetonitrile

2-((2S)-4-(7-(2-cyclopropylphenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (10 mg, 0.018 mmol) and (Z)-2-fluoro-3-(thiazol-2-yl)acrylic acid (9.5 mg, 0.055 mmol)were combined as solids and dissolved in DMF (600 μL). 1-methylimidazole (8.8 μL, 0.11 mmol) was added followed by chloro-N,N,N′,N′-tetramethylformamidinium hexafluorophosphate (15 mg, 0.055 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 73% 5:95 MeCN:H₂O with 10 mM AA/27% 95:5 MeCN:H₂O with 10 mM AA→100% 95:5 MeCN:H₂O with 10 mM AA; λ=220 nm) to provide the desired product (9.1 mg, 0.013 mmol, 71% yield) as an off-white solid. LCMS (ESI) m/z: [M+H]⁺ calcd for C₃₆H₃₆F₃N₈O₂ S 701.3; found 701.3; ¹H NMR (500 MHz, DMSO-d₆) δ 9.22 (s, 1H), 8.06-7.99 (m, 2H), 7.42-7.38 (m, 1H), 7.35-7.26 (m, 2H), 7.10 (d, J=39.4 Hz, 1H), 7.05-7.03 (m, 1H), 5.28 (d, J=54.2 Hz, 1H), 4.96-4.84 (m, 1H), 4.58-4.49 (m, 1H), 4.48-4.39 (m, 1H), 4.16 (d, J=10.4 Hz, 1H), 4.08 (br d, J=10.4 Hz, 1H), 4.24-4.04 (m, 1H), 3.87-3.72 (m, 2H), 3.18-3.17 (m, 1H), 3.14-3.00 (m, 5H), 2.86-2.80 (m, 1H), 2.17-1.96 (m, 3H), 1.88-1.74 (m, 4H), 0.77 (br d, J=8.3 Hz, 2H), 0.63 (br d, J=5.0 Hz, 2H).

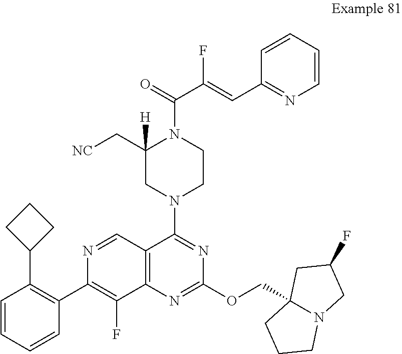 [SMILES: N#CC[C@H]1CN(c2nc(OC[C@@]34CCCN3C[C@H](F)C4)nc3c(F)c(-c4ccccc4C4CCC4)ncc23)CCN1C(=O)/C(F)=C/c1ccccn1]

### 2-((2S)-4-(7-(2-cyclobutylphenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(pyridin-2-yl)acryloyl)piperazin-2-yl)acetonitrile

2-((2S)-4-(7-(2-Cyclobutylphenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (10 mg, 0.018 mmol) and (Z)-2-fluoro-3-(pyridin-2-yl)acrylic acid (9.0 mg, 0.054 mmol) were combined as solids and dissolved in DMF (600 μL). 1-methylimidazole (8.8 μL, 0.11 mmol) was added followed by chloro-N,N,N′,N′-tetramethylformamidinium hexafluorophosphate (15 mg, 0.054 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 69% 5:95 MeCN:H₂O with 10 mM AA/31% 95:5 MeCN:H₂O with 10 mM AA→100% 95:5 MeCN:H₂O with 10 mM AA; λ=220 nm) to provide the desired product (11.9 mg, 0.017 mmol, 94% yield) as an off-white solid. LCMS (ESI) m/z: [M+H]⁺ calcd for C₃₉H₄₀F₃N₈O₂ 709.3; found 709.4; ¹H NMR (500 MHz, DMSO-d₆) δ 9.18 (s, 1H), 8.65 (br d, J=3.7 Hz, 1H), 7.92-7.88 (m, 1H), 7.80 (d, J=7.9 Hz, 1H), 7.52-7.46 (m, 2H), 7.40-7.29 (m, 3H), 6.66 (d, J=38.1 Hz, 1H), 5.28 (d, J=55.0 Hz, 1H), 4.98-4.83 (m, 1H), 4.56-4.49 (m, 1H), 4.48-4.40 (m, 1H), 4.16 (br d, J=10.4 Hz, 1H), 4.08 (br d, J=10.5 Hz, 1H), 4.21-4.05 (m, 1H), 3.87-3.71 (m, 2H), 3.63-3.59 (m, 1H), 3.14-3.00 (m, 5H), 2.88-2.79 (m, 1H), 2.18-1.92 (m, 7H), 1.87-1.61 (m, 6H).

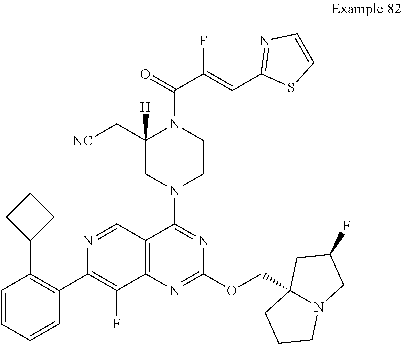 [SMILES: N#CC[C@H]1CN(c2nc(OC[C@@]34CCCN3C[C@H](F)C4)nc3c(F)c(-c4ccccc4C4CCC4)ncc23)CCN1C(=O)/C(F)=C/c1nccs1]

### 2-((2S)-4-(7-(2-cyclobutylphenyl)-8-fluoro-2-(((2R, 7aS)-2-fluorotetrahydro-1H-pyrrolizin-7a(5H)-yl)methoxy)pyrido[4,3-d]pyrimidin-4-yl)-1-((Z)-2-fluoro-3-(thiazol-2-yl)acryloyl)piperazin-2-yl)acetonitrile

2-((2S)-4-(7-(2-Cyclobutylphenyl)-8-fluoro-2-(((2R, 7aS)-2-fluorotetrahydro-1H-pyrrolizin-7a(5H)-yl)methoxy)pyrido[4,3-d]pyrimidin-4-yl)piperazin-2-yl)acetonitrile (10 mg, 0.018 mmol) were combined as solids and dissolved in DMF (600 μL). 1-methylimidazole (8.8 μL, 0.11 mmol) was added followed by chloro-N,N,N′,N′-tetramethylformamidinium hexafluorophosphate (15 mg, 0.054 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 68% 5:95 MeCN:H₂O with 10 mM AA/32% 95:5 MeCN:H₂O with 10 mM AA→100% 95:5 MeCN:H₂O with 10 mM AA; λ=220 nm) to provide the desired product (12 mg, 0.017 mmol, 94% yield) as an off-white solid. LCMS (ESI) m/z: [M+H]⁺ calcd for C₃₇H₃₈F₃N₈O₂ S 715.3; found 715.4; ¹H NMR (500 MHz, DMSO-d₆) δ 9.18 (s, 1H), 8.02 (dd, J=6.4, 2.5 Hz, 2H), 7.53-7.46 (m, 2H), 7.38-7.29 (m, 2H), 7.10 (d, J=39.4 Hz, 1H), 5.28 (d, J=54.1 Hz, 1H), 4.96-4.84 (m, 1H), 4.52 (br d, J=12.3 Hz, 1H), 4.48-4.39 (m, 1H), 4.16 (d, J=10.4 Hz, 1H), 4.08 (br d, J=10.5 Hz, 1H), 4.24-4.03 (m, 1H), 3.86-3.74 (m, 2H), 3.65-3.58 (m, 1H), 3.14-3.02 (m, 5H), 2.87-2.79 (m, 1H), 2.17-1.92 (m, 7H), 1.88-1.63 (m, 6H).

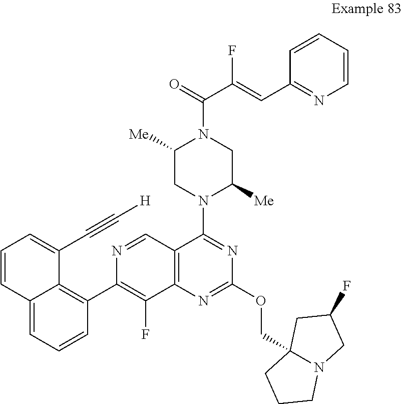 [SMILES: C#Cc1cccc2cccc(-c3ncc4c(N5C[C@H](C)N(C(=O)/C(F)=C/c6ccccn6)C[C@H]5C)nc(OC[C@@]56CCCN5C[C@H](F)C6)nc4c3F)c12]

### (Z)-1-((2S,5R)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-2,5-dimethylpiperazin-1-yl)-2-fluoro-3-(pyridin-2-yl)prop-2-en-1-one

4-((2R, 5S)-2,5-Dimethylpiperazin-1-yl)-7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidine (7.5 mg, 0.013 mmol) and (Z)-2-fluoro-3-(pyridin-2-yl)acrylic acid (6.6 mg, 0.040 mmol) were combined as solids and dissolved in DMF (600 μL). 1-methylimidazole (6.3 μL, 0.079 mmol) was added followed by chloro-N,N,N′,N′-tetramethylformamidinium hexafluorophosphate (11 mg, 0.040 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 69% 5:95 MeCN:H₂O with 10 mM AA/31% 95:5 MeCN:H₂O with 10 mM AA→100% 95:5 MeCN:H₂O with 10 mM AA; λ=220 nm) to provide the desired product (2.3 mg, 0.003 mmol, 24% yield) as a brown solid. LCMS (ESI) m/z: [M+H]⁺ calcd for C₄₁H₃₉F₃N₇O₂ 718.3; found 718.2; ¹H NMR (500 MHz, DMSO-d₆) δ 9.11-8.99 (m, 1H), 8.69-8.61 (m, 1H), 8.23-8.10 (m, 2H), 7.96-7.86 (m, 1H), 7.81-7.76 (m, 1H), 7.76-7.67 (m, 2H), 7.64-7.54 (m, 2H), 7.41-7.35 (m, 1H), 6.74-6.53 (m, 1H), 5.28 (d, J=54.5 Hz, 1H), 5.08-4.45 (m, 2H), 4.44-4.33 (m, 1H), 4.24-4.02 (m, 2H), 3.97-3.80 (m, 3H), 3.20-3.01 (m, 4H), 2.89-2.78 (m, 1H), 2.19-1.97 (m, 3H), 1.88-1.74 (m, 3H), 1.51-1.25 (m, 6H).

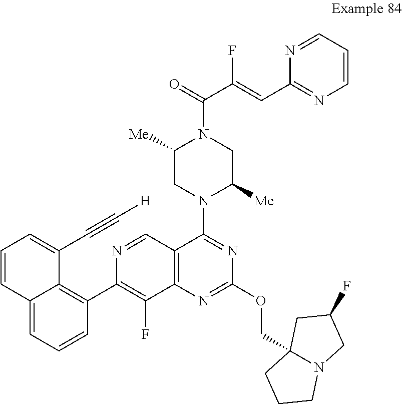 [SMILES: C#Cc1cccc2cccc(-c3ncc4c(N5C[C@H](C)N(C(=O)/C(F)=C/c6ncccn6)C[C@H]5C)nc(OC[C@@]56CCCN5C[C@H](F)C6)nc4c3F)c12]

### (Z)-1-((3R)-4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyrimidin-2-yl)prop-2-en-1-one

4-((2R, 5S)-2,5-Dimethylpiperazin-1-yl)-7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidine (7.5 mg, 0.013 mmol) and (Z)-2-fluoro-3-(pyrimidin-2-yl)acrylic acid (6.7 mg, 0.040 mmol) were combined as solids and dissolved in DMF (600 μL). 1-methylimidazole (6.3 L, 0.079 mmol) was added followed by chloro-N,N,N′,N′-tetramethylformamidinium hexafluorophosphate (11 mg, 0.040 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (2.5 mg, 0.003 mmol, 26% yield) as a brown solid. LCMS (ESI) m/z: [M+H]⁺ calcd for C₄₀H₃₈F₃NsO₂ 719.3; found 719.2; ¹H NMR (500 MHz, DMSO-d₆) δ 9.12-8.99 (m, 1H), 8.89 (d, J=4.7 Hz, 2H), 8.21-8.10 (m, 2H), 7.76-7.68 (m, 2H), 7.65-7.55 (m, 2H), 7.45 (t, J=4.8 Hz, 1H), 6.71-6.49 (m, 1H), 5.28 (d, J=53.6 Hz, 1H), 5.09-4.81 (m, 1H), 4.76-4.36 (m, 2H), 4.16 (br d, J=10.1 Hz, 1H), 4.07 (d, J=10.2 Hz, 1H), 3.99-3.91 (m, 1H), 3.89-3.72 (m, 2H), 3.03 (br s, 4H), 2.83 (br d, J=7.4 Hz, 1H), 2.18-1.97 (m, 3H), 1.87-1.74 (m, 3H), 1.49-1.25 (m, 6H).

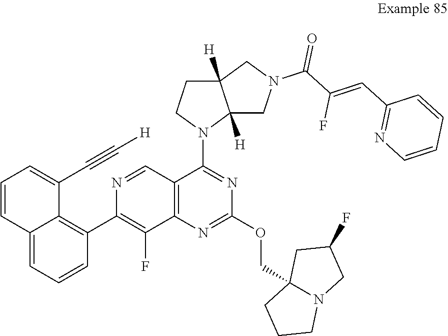 [SMILES: C#Cc1cccc2cccc(-c3ncc4c(N5CC[C@@H]6CN(C(=O)/C(F)=C/c7ccccn7)C[C@@H]65)nc(OC[C@@]56CCCN5C[C@H](F)C6)nc4c3F)c12]

### (Z)-1-((3aR, 6aR)-1-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)hexahydropyrrolo[3,4-b]pyrrol-5(1H)-yl)-2-fluoro-3-(pyridin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-((3aR, 6aR)-hexahydropyrrolo[3,4-b]pyrrol-1(2H)-yl)pyrido[4,3-d]pyrimidine (11 mg, 0.019 mmol) were combined as solids and dissolved in DMF (600 μL). 1-methylimidazole (9.3 μL, 0.12 mmol) was added followed by chloro-N,N,N′,N′-tetramethylformamidinium hexafluorophosphate (16 mg, 0.058 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 78% 5:95 MeCN:H₂O with 10 mM AA/22% 95:5 MeCN:H₂O with 10 mM AA→100% 95:5 MeCN:H₂O with 10 mM AA; λ=220 nm) to provide the desired product (10.9 mg, 0.014 mmol, 72% yield) as a brown solid. LCMS (ESI) m/z: [M+H]⁺ calcd for C₄₁H₃₇F₃N₇O₂ 716.3; found 716.2; ¹H NMR (500 MHz, DMSO-d₆) δ 9.29-9.17 (m, 1H), 8.70-8.54 (m, 1H), 8.14 (br t, J=9.5 Hz, 2H), 7.92-7.68 (m, 4H), 7.60-7.32 (m, 3H), 6.86-6.66 (m, 1H), 5.39-5.14 (m, 1H), 5.05-4.90 (m, 1H), 4.53-4.26 (m, 1H), 4.26-3.99 (m, 5H), 3.93-3.63 (m, 3H), 3.14-2.95 (m, 4H), 2.90-2.75 (m, 1H), 2.30-1.97 (m, 5H), 1.85-1.62 (m, 3H).

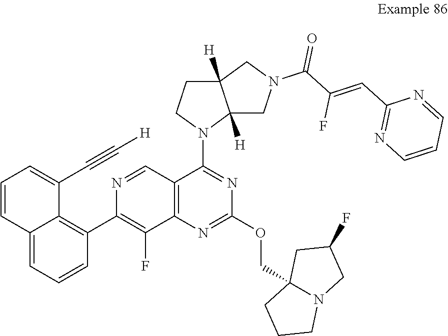 [SMILES: C#Cc1cccc2cccc(-c3ncc4c(N5CC[C@@H]6CN(C(=O)/C(F)=C/c7ncccn7)C[C@@H]65)nc(OC[C@@]56CCCN5C[C@H](F)C6)nc4c3F)c12]

### (Z)-1-((3aR, 6aR)-1-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)hexahydropyrrolo[3,4-b]pyrrol-5(1H)-yl)-2-fluoro-3-(pyrimidin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-((3aR, 6aR)-hexahydropyrrolo[3,4-b]pyrrol-1(2H)-yl)pyrido[4,3-d]pyrimidine (11 mg, 0.019 mmol) and (Z)-2-fluoro-3-(pyrimidin-2-yl)acrylic acid (9.7 mg, 0.058 mmol) were combined as solids and dissolved in DMF (600 L). 1-methylimidazole (9.3 μL, 0.12 mmol) was added followed by chloro-N,N,N′,N′-tetramethylformamidinium hexafluorophosphate (16 mg, 0.058 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (10.9 mg, 0.014 mmol, 72% yield) as a brown solid. LCMS (ESI) m/z: [M+H]⁺ calcd for C₄₀H₃₆F₃N₈O₂ 717.3; found 717.2; ¹H NMR (500 MHz, DMSO-d₆) δ 9.27-9.17 (m, 1H), 8.94-8.80 (m, 2H), 8.14 (br s, 2H), 7.75-7.65 (m, 2H), 7.56 (br s, 2H), 7.47-7.38 (m, 1H), 6.84-6.61 (m, 1H), 5.37-5.18 (m, 1H), 5.04-4.93 (m, 1H), 4.46-4.35 (m, 2H), 4.20-4.00 (m, 5H), 3.88-3.78 (m, 2H), 3.14-2.95 (m, 4H), 2.88-2.73 (m, 1H), 2.30-2.00 (m, 5H), 1.83-1.72 (m, 3H).

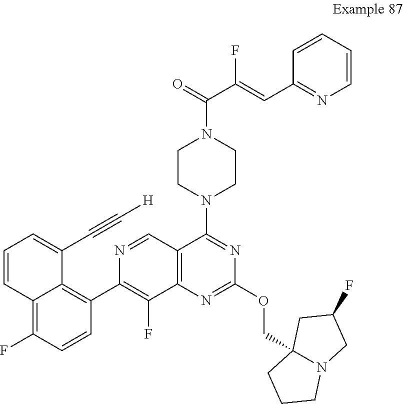 [SMILES: C#Cc1cccc2c(F)ccc(-c3ncc4c(N5CCN(C(=O)/C(F)=C/c6ccccn6)CC5)nc(OC[C@@]56CCCN5C[C@H](F)C6)nc4c3F)c12]

### (Z)-1-(4-(7-(8-ethynyl-4-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridin-2-yl)prop-2-en-1-one

7-(8-Ethynyl-4-fluoronaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (5 mg, 8.95 μmol) and (Z)-2-fluoro-3-(pyridin-2-yl)acrylic acid (4.5 mg, 0.027 mmol) were combined as solids and dissolved in DMF (400 μL). 1-methylimidazole (4.3 μL, 0.054 mmol) was added followed by chloro-N,N,N′,N′-tetramethylformamidinium hexafluorophosphate (7.5 mg, 0.027 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 71% 5:95 MeCN:H₂O with 10 mM AA/29% 95:5 MeCN:H₂O with 10 mM AA→100% 95:5 MeCN:H₂O with 10 mM AA; λ=220 nm) to provide the desired product (4.6 mg, 6.50 μmol, 73% yield) as a brown solid. LCMS (ESI) m/z: [M+H]⁺ calcd for C₃₉H₃₄F₄N₇O₂ 708.3; found 708.2; ¹H NMR (500 MHz, DMSO-d₆) δ 9.12 (s, 1H), 8.65 (br d, J=4.2 Hz, 1H), 8.26 (br d, J=8.2 Hz, 1H), 7.94-7.86 (m, 1H), 7.85-7.77 (m, 2H), 7.69 (t, J=7.7 Hz, 1H), 7.66-7.61 (m, 1H), 7.59-7.53 (m, 1H), 7.38 (dd, J=7.1, 5.2 Hz, 1H), 6.66 (d, J=39.0 Hz, 1H), 5.28 (d, J=54.8 Hz, 1H), 4.20-3.86 (m, 9H), 3.82 (s, 1H), 3.13-3.01 (m, 4H), 2.87-2.79 (m, 1H), 2.15-1.96 (m, 3H), 1.83-1.72 (m, 3H).

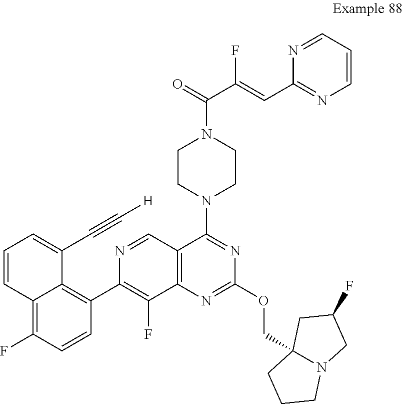 [SMILES: C#Cc1cccc2c(F)ccc(-c3ncc4c(N5CCN(C(=O)/C(F)=C/c6ncccn6)CC5)nc(OC[C@@]56CCCN5C[C@H](F)C6)nc4c3F)c12]

### (Z)-1-(4-(7-(8-ethynyl-4-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyrimidin-2-yl)prop-2-en-1-one

7-(8-Ethynyl-4-fluoronaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (5 mg, 8.95 μmol) and (Z)-2-fluoro-3-(pyrimidin-2-yl)acrylic acid (4.5 mg, 0.027 mmol) were combined as solids and dissolved in DMF (40 μL). 1-methylimidazole (4.3 μL, 0.054 mmol) was added followed by chloro-N,N,N′,N′-tetramethylformamidinium hexafluorophosphate (7.5 mg, 0.027 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (4.3 mg, 6.0 μmol, 67% yield) as a brown solid. LCMS (ESI) m/z: [M+H]⁺ calcd for C₃₈H₃₃F₄N₈O₂ 709.3; found 709.3; ¹H NMR (500 MHz, DMSO-d₆) δ 9.11 (s, 1H), 8.89 (d, J=4.9 Hz, 2H), 8.26 (d, J=8.5 Hz, 1H), 7.83 (d, J=7.2 Hz, 1H), 7.69 (t, J=7.7 Hz, 1H), 7.65-7.60 (m, 1H), 7.56 (br dd, J=9.8, 8.7 Hz, 1H), 7.44 (t, J=4.9 Hz, 1H), 6.63 (d, J=34.9 Hz, 1H), 5.28 (d, J=54.3 Hz, 1H), 4.19-3.83 (m, 10H), 3.81 (s, 1H), 3.14-3.01 (m, 3H), 2.87-2.79 (m, 1H), 2.17-1.94 (m, 3H), 1.88-1.75 (m, 3H).

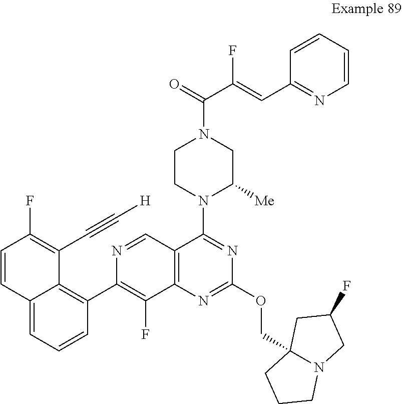 [SMILES: C#Cc1c(F)ccc2cccc(-c3ncc4c(N5CCN(C(=O)/C(F)=C/c6ccccn6)C[C@@H]5C)nc(OC[C@@]56CCCN5C[C@H](F)C6)nc4c3F)c12]

### (Z)-1-((S)-4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyridin-2-yl)prop-2-en-1-one

7-(8-Ethynyl-7-fluoronaphthalen-1-yl)-8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidine (9 mg, 0.016 mmol) and (Z)-2-fluoro-3-(pyridin-2-yl)acrylic acid (7.9 mg, 0.047 mmol) were combined as solids and dissolved in DMF (700 μL). 1-methylimidazole (7.5 μL, 0.094 mmol) was added followed by chloro-N,N,N′,N′-tetramethylformamidinium hexafluorophosphate (13 mg, 0.047 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 71% 5:95 MeCN:H₂O with 10 mM AA/29% 95:5 MeCN:H₂O with 10 mM AA→100% 95:5 MeCN:H₂O with 10 mM AA; λ=220 nm) to provide the desired product (6.1 mg, 8.4 μmol, 53% yield) as a brown solid. LCMS (ESI) m/z: [M+H]⁺ calcd for C₄₀H₃₆F₄N₇O₂ 722.3; found 722.4; ¹H NMR (500 MHz, DMSO-d₆) δ 9.12-9.00 (m, 1H), 8.71-8.57 (m, 1H), 8.31-8.16 (m, 2H), 7.99-7.85 (m, 1H), 7.84-7.76 (m, 1H), 7.73-7.56 (m, 3H), 7.42-7.33 (m, 1H), 6.72-6.54 (m, 1H), 5.27 (d, J=53.7 Hz, 1H), 5.10-4.84 (m, 1H), 4.53-4.31 (m, 1H), 4.26-3.99 (m, 7H), 3.90 (s, 1H), 3.13-3.00 (m, 3H), 2.87-2.77 (m, 1H), 2.19-1.94 (m, 3H), 1.87-1.69 (m, 3H), 1.53-1.35 (m, 3H).

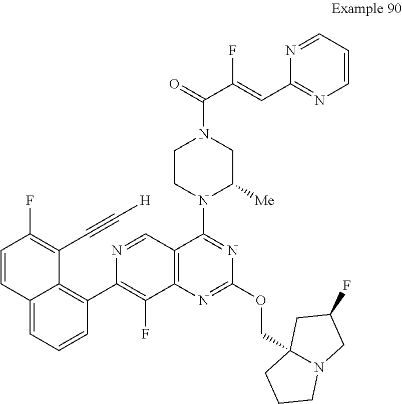 [SMILES: C#Cc1c(F)ccc2cccc(-c3ncc4c(N5CCN(C(=O)/C(F)=C/c6ncccn6)C[C@@H]5C)nc(OC[C@@]56CCCN5C[C@H](F)C6)nc4c3F)c12]

### (Z)-1-((S)-4-(7-(8-ethynyl-7-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-3-methylpiperazin-1-yl)-2-fluoro-3-(pyrimidin-2-yl)prop-2-en-1-one

7-(8-Ethynyl-7-fluoronaphthalen-1-yl)-8-fluoro-2-(((2R, 7aS)-2-fluorotetrahydro-1H-pyrrolizin-7a(5H)-yl)methoxy)-4-((S)-2-methylpiperazin-1-yl)pyrido[4,3-d]pyrimidine (9 mg, 0.016 mmol) and (Z)-2-fluoro-3-(pyrimidin-2-yl)acrylic acid (7.9 mg, 0.047 mmol) were combined as solids and dissolved in DMF (700 μL). 1-methylimidazole (7.5 μL, 0.094 mmol) was added followed by chloro-N,N,N′,N′-tetramethylformamidinium hexafluorophosphate (13 mg, 0.047 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (5.6 mg, 7.7 μmol, 49% yield) as a brown solid. LCMS (ESI) m/z: [M+H]⁺ calcd for C₃₉H₃₅F₄N₈O₂ 723.3; found 723.4; ¹H NMR (500 MHz, DMSO-d₆) δ 9.12-9.01 (m, 1H), 8.89 (d, J=4.9 Hz, 2H), 8.31-8.16 (m, 2H), 7.73-7.57 (m, 3H), 7.45 (t, J=4.9 Hz, 1H), 6.74-6.52 (m, 1H), 5.28 (d, J=54.2 Hz, 1H), 5.10-4.86 (m, 1H), 4.51-4.32 (m, 1H), 4.21-4.01 (m, 5H), 3.97-3.75 (m, 2H), 3.13-2.99 (m, 4H), 2.88-2.76 (m, 1H), 2.20-1.95 (m, 3H), 1.87-1.69 (m, 3H), 1.52-1.38 (m, 3H).

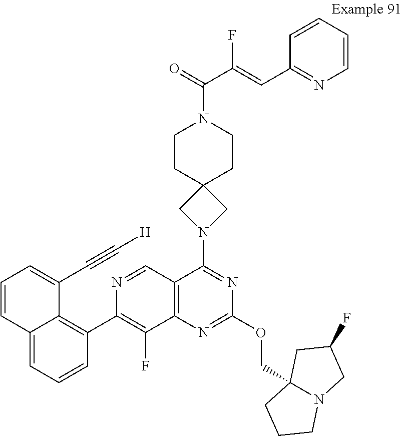 [SMILES: C#Cc1cccc2cccc(-c3ncc4c(N5CC6(CCN(C(=O)/C(F)=C/c7ccccn7)CC6)C5)nc(OC[C@@]56CCCN5C[C@H](F)C6)nc4c3F)c12]

### (Z)-1-(2-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-2,7-diazaspiro[3.5]nonan-7-yl)-2-fluoro-3-(pyridin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-(2,7-diazaspiro[3.5]nonan-2-yl)pyrido[4,3-d]pyrimidine (5 mg, 8.6 μmol) and (Z)-2-fluoro-3-(pyridin-2-yl)acrylic acid (4.3 mg, 0.026 mmol) were combined as solids and dissolved in DMF (400 μL). 1-methylimidazole (4.1 μL, 0.052 mmol) was added followed by chloro-N,N,N′,N′-tetramethylformamidinium hexafluorophosphate (11 mg, 0.040 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide the desired product (3.4 mg, 4.6 μmol, 54% yield) as a brown solid. LCMS (ESI) m/z: [M+H]⁺ calcd for C₄₂H₃₉F₃N₇O₂ 730.3; found 730.3; ¹H NMR (500 MHz, DMSO-d₆) δ 8.96 (s, 1H), 8.63 (br d, J=4.8 Hz, 1H), 8.18-8.11 (m, 2H), 7.87 (td, J=7.7, 1.7 Hz, 1H), 7.76 (d, J=8.1 Hz, 1H), 7.71 (s, 2H), 7.59-7.52 (m, 2H), 7.36 (dd, J=6.7, 4.9 Hz, 2H), 6.51 (d, J=39.2 Hz, 1H), 5.35 (d, J=53.9 Hz, 1H), 4.74-4.58 (m, 2H), 4.13 (br s, 4H), 3.74-3.58 (m, 6H), 3.01-2.91 (m, 1H), 2.35-2.02 (m, 3H), 1.97-1.81 (m, 8H).

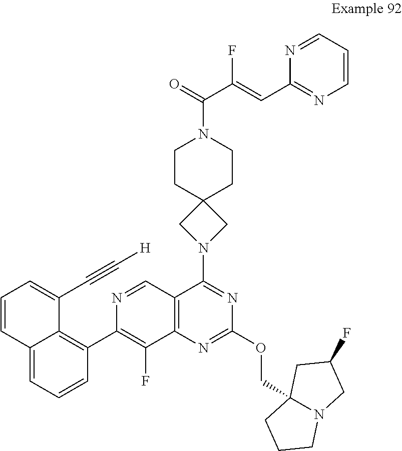 [SMILES: C#Cc1cccc2cccc(-c3ncc4c(N5CC6(CCN(C(=O)/C(F)=C/c7ncccn7)CC6)C5)nc(OC[C@@]56CCCN5C[C@H](F)C6)nc4c3F)c12]

### (Z)-1-(2-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-2,7-diazaspiro[3.5]nonan-7-yl)-2-fluoro-3-(pyrimidin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-(2,7-diazaspiro[3.5]nonan-2-yl)pyrido[4,3-d]pyrimidine (5 mg, 8.6 μmol)and (Z)-2-fluoro-3-(pyrimidin-2-yl)acrylic acid (4.3 mg, 0.026 mmol) were combined as solids and dissolved in DMF (400 μL). 1-methylimidazole (4.1 μL, 0.052 mmol) was added followed by chloro-N,N,N′,N′-tetramethylformamidinium hexafluorophosphate (11 mg, 0.040 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide the desired product (3.2 mg, 4.3 μmol, 50% yield) as a brown solid. LCMS (ESI) m/z: [M+H]⁺ calcd for C₄₁H₃₈F₃N₈O₂ 731.3; found 731.3; ¹H NMR (500 MHz, DMSO-d₆) δ 8.94 (br s, 1H), 8.85-8.81 (m, 2H), 8.20-8.05 (m, 2H), 7.77-7.65 (m, 2H), 7.62-7.52 (m, 2H), 7.40 (td, J=4.9, 1.6 Hz, 1H), 6.47 (d, J=35.8 Hz, 1H), 5.38 (d, J=53.3 Hz, 1H), 4.76-4.54 (m, 2H), 4.37-4.17 (m, 4H), 3.56-3.33 (m, 7H), 3.09-2.92 (m, 1H), 2.31-1.81 (m, 11H).

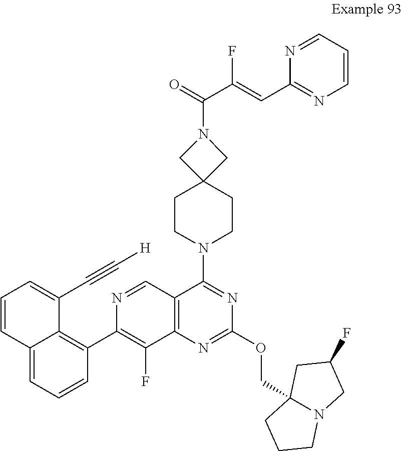 [SMILES: C#Cc1cccc2cccc(-c3ncc4c(N5CCC6(CC5)CN(C(=O)/C(F)=C/c5ncccn5)C6)nc(OC[C@@]56CCCN5C[C@H](F)C6)nc4c3F)c12]

### (Z)-1-(7-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-2,7-diazaspiro[3.5]nonan-2-yl)-2-fluoro-3-(pyridin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-(2,7-diazaspiro[3.5]nonan-2-yl)pyrido[4,3-d]pyrimidine (5 mg, 8.6 μmol) were combined as solids and dissolved in DMF (400 μL). 1-methylimidazole (4.1 μL, 0.052 mmol) was added followed by chloro-N,N,N′,N′-tetramethylformamidinium hexafluorophosphate (11 mg, 0.040 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 71% 5:95 MeCN:H₂O with 10 mM AA/29% 95:5 MeCN:H₂O with 10 mM AA→100% 95:5 MeCN:H₂O with 10 mM AA; λ=220 nm) to provide the desired product (2.3 mg, 3.10 μmol, 36% yield) as a brown solid. LCMS (ESI) m/z: [M+H]⁺ calcd for C₄₂H₃₉F₃N₇O₂ 730.3; found 730.3; ¹H NMR (500 MHz, DMSO-d₆) δ 9.02 (s, 1H), 8.65 (br d, J=4.7 Hz, 1H), 8.14 (dd, J=11.6, 8.0 Hz, 2H), 7.88 (td, J=7.7, 1.2 Hz, 1H), 7.76 (d, J=8.2 Hz, 1H), 7.73-7.68 (m, 2H), 7.64-7.54 (m, 2H), 7.38 (ddd, J=7.4, 4.8, 0.8 Hz, 1H), 6.85 (d, J=37.5 Hz, 1H), 5.35 (d, J=53.2 Hz, 1H), 4.39-3.82 (m, 10H), 3.21-3.10 (m, 3H), 3.02-2.86 (m, 1H), 2.31-1.81 (m, 11H).

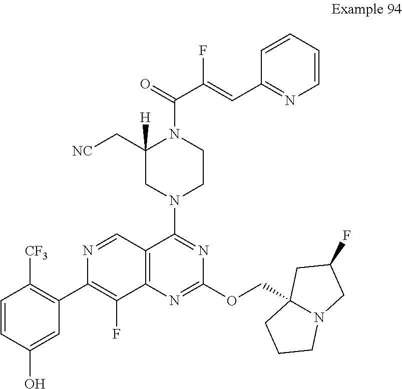 [SMILES: N#CC[C@H]1CN(c2nc(OC[C@@]34CCCN3C[C@H](F)C4)nc3c(F)c(-c4cc(O)ccc4C(F)(F)F)ncc23)CCN1C(=O)/C(F)=C/c1ccccn1]

### 2-((S)-4-(8-fluoro-2-(((2R, 7aS)-2-fluorotetrahydro-1H-pyrrolizin-7a(5H)-yl)methoxy)-7-(5-hydroxy-2-(trifluoromethyl)phenyl)pyrido[4,3-d]pyrimidin-4-yl)-1-((Z)-2-fluoro-3-(pyridin-2-yl)acryloyl)piperazin-2-yl)acetonitrile

2-((S)-4-(8-fluoro-2-(((2R, 7aS)-2-fluorotetrahydro-1H-pyrrolizin-7a(5H)-yl)methoxy)-7-(5-hydroxy-2-(trifluoromethyl)phenyl)pyrido[4,3-d]pyrimidin-4-yl)piperazin-2-yl)acetonitrile (5 mg, 8.48 μmol) and (Z)-2-fluoro-3-(pyridin-2-yl)acrylic acid (4.2 mg, 0.025 mmol) were combined as solids and dissolved in DMF (400 μL). 1-methylimidazole (3.5 μL, 0.042 mmol) was added followed by chloro-N,N,N′,N′-tetramethylformamidinium hexafluorophosphate (7.1 mg, 0.025 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (1.9 mg, 2.5 μmol, 29% yield) as an off-white solid. LCMS (ESI) m/z: [M+H]⁺ calcd for C₃₄H₃₁F₆N₈O₃S 739.2; found 739.2; ¹H NMR (500 MHz, DMSO-d₆) δ 10.62 (br s, 1H), 9.17 (s, 1H), 8.65 (br d, J=4.5 Hz, 1H), 7.90 (td, J=7.8, 1.3 Hz, 1H), 7.79 (d, J=8.2 Hz, 1H), 7.71 (d, J=8.8 Hz, 1H), 7.39 (dd, J=7.2, 4.5 Hz, 1H), 7.06 (dd, J=9.1, 1.9 Hz, 1H), 6.86 (d, J=2.1 Hz, 1H), 6.66 (d, J=39.5 Hz, 1H), 5.30 (d, J=56.2 Hz, 1H), 4.99-4.81 (m, 1H), 4.61-4.38 (m, 2H), 4.27-4.04 (m, 3H), 4.01-3.67 (m, 3H), 3.21-3.05 (m, 5H), 2.94-2.80 (m, 1H), 2.23-1.99 (m, 3H), 1.89-1.76 (m, 3H).

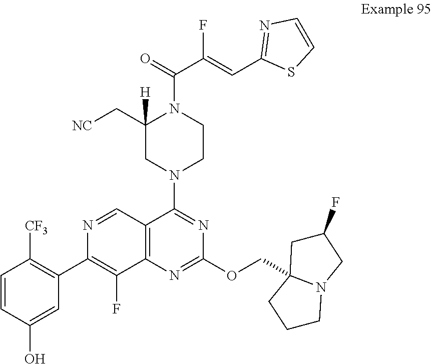

### 2-((S)-4-(8-fluoro-2-(((2R, 7aS)-2-fluorotetrahydro-1H-pyrrolizin-7a(5H)-yl)methoxy)-7-(5-hydroxy-2-(trifluoromethyl)phenyl)pyrido[4,3-d]pyrimidin-4-yl)-1-((Z)-2-fluoro-3-(thiazol-2-yl)acryloyl)piperazin-2-yl)acetonitrile

2-((S)-4-(8-Fluoro-2-(((2R, 7aS)-2-fluorotetrahydro-1H-pyrrolizin-7a(5H)-yl)methoxy)-7-(5-hydroxy-2-(trifluoromethyl)phenyl)pyrido[4,3-d]pyrimidin-4-yl)piperazin-2-yl)acetonitrile (5 mg, 8.48 μmol) and (Z)-2-fluoro-3-(thiazol-2-yl)acrylic acid (4.4 mg, 0.025 mmol) were combined as solids and dissolved in DMF (400 μL). 1-methylimidazole (3.5 μL, 0.042 mmol) was added followed by chloro-N,N,N′,N′-tetramethylformamidinium hexafluorophosphate (7.1 mg, 0.025 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (1.8 mg, 2.303 μmol, 27% yield) as an off-white solid. LCMS (ESI) m/z: [M+H]⁺ calcd for C₃₄H₃₁F₆N₈O₃S 745.2; found 745.2; ¹H NMR (500 MHz, DMSO-d₆) δ 10.63 (br s, 1H), 9.18 (s, 1H), 8.02 (dd, J=6.9, 2.7 Hz, 2H), 7.71 (d, J=8.8 Hz, 1H), 7.10 (d, J=39.0 Hz, 1H), 7.09-7.04 (m, 1H), 6.86 (d, J=2.0 Hz, 1H), 5.36 (d, J=52.7 Hz, 1H), 4.98-4.79 (m, 1H), 4.61-4.38 (m, 2H), 4.247-4.04 (m, 3H), 3.89-3.69 (m, 3H), 3.25-3.08 (m, 5H), 3.02-2.90 (m, 1H), 2.31-2.03 (m 3H) 2.00-1.92 (m, 1H), 1.90-1.79 (m, 2H).

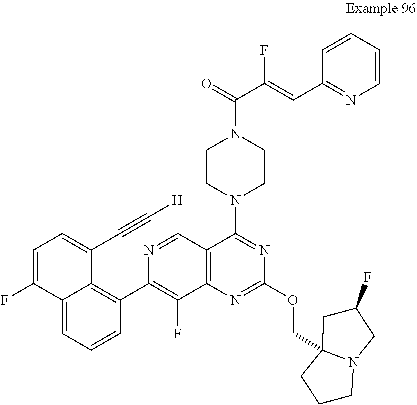

### (Z)-1-(4-(7-(8-ethynyl-5-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridin-2-yl)prop-2-en-1-one

7-(8-Ethynyl-5-fluoronaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (7 mg, 0.013 mmol) were combined as solids and dissolved in DMF (600 μL). 1-methylimidazole (6.0 μL, 0.075 mmol) was added followed by chloro-N,N,N′,N′-tetramethylformamidinium hexafluorophosphate (11 mg, 0.038 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 71% 5:95 MeCN:H₂O with 10 mM AA/29% 95:5 MeCN:H₂O with 10 mM AA→100% 95:5 MeCN:H₂O with 10 mM AA; λ=220 nm) to provide the desired product (4.1 mg, 5.8 μmol, 46% yield) as a brown solid. LCMS (ESI) m/z: [M+H]⁺ calcd for C₃₉H₃₄F₄N₇O₂ 708.3; found 708.6; ¹H NMR (500 MHz, DMSO-d₆) δ 9.14 (s, 1H), 8.65 (br d, J=5.1 Hz, 1H), 8.32-8.26 (m, 1H), 7.89 (td, J=7.7, 1.6 Hz, 1H), 7.85-7.65 (m, 4H), 7.48-7.42 (m, 1H), 7.38 (dd, J=7.1, 5.1 Hz, 1H), 6.66 (d, J=38.8 Hz, 1H), 5.32 (d, J=52.4 Hz, 1H), 4.22-3.91 (m, 10H), 3.71 (s, 1H), 3.23-3.09 (m, 3H), 3.00-2.83 (m, 1H), 2.26-2.01 (m, 3H), 1.89-1.75 (m, 3H).

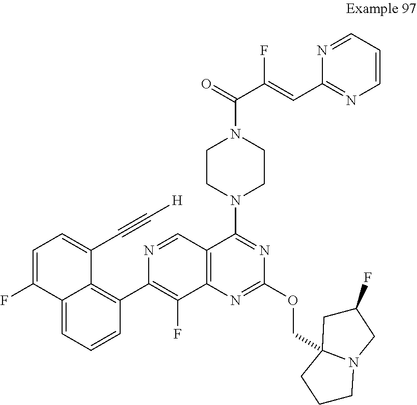

### (Z)-1-(4-(7-(8-ethynyl-5-fluoronaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyrimidin-2-yl)prop-2-en-1-one

7-(8-Ethynyl-5-fluoronaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (7 mg, 0.013 mmol) and (Z)-2-fluoro-3-(pyrimidin-2-yl)acrylic acid (6.3 mg, 0.038 mmol) were combined as solids and dissolved in DMF (600 μL). 1-methylimidazole (6.0 μL, 0.075 mmol) was added followed by chloro-N,N,N′,N′-tetramethylformamidinium hexafluorophosphate (11 mg, 0.038 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (2.1 mg, 2.8 μmol, 22% yield) as a brown solid. LCMS (ESI) m/z: [M+H]⁺ calcd for C₃₈H₃₃F₄N₈O₂ 709.3; found 709.1; ¹H NMR (500 MHz, DMSO-d₆) δ 9.14 (s, 1H), 8.89 (d, J=4.9 Hz, 2H), 8.30 (d, J=8.7 Hz, 1H), 7.83 (dd, J=8.3, 7.6 Hz, 1H), 7.77-7.71 (m, 2H), 7.48-7.41 (m, 2H), 6.64 (d, J=35.1 Hz, 1H), 5.35 (d, J=54.1 Hz, 1H), 4.31-3.93 (m, 9H), 3.88-3.81 (m, 1H), 3.70 (s, 1H), 3.21-3.09 (m, 3H), 2.99-2.88 (m, 1H), 2.29-2.01 (m, 3H), 1.97-1.81 (m, 3H).

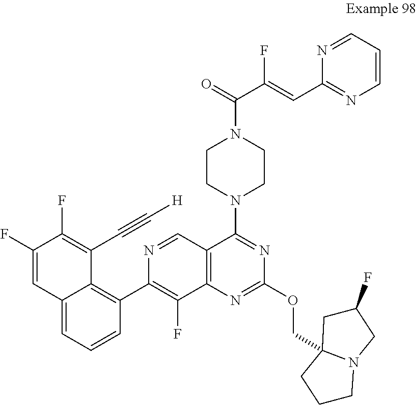

### (Z)-1-(4-(7-(8-ethynyl-6,7-difluoronaphthalen-1-yl)-8-fluoro-2-(((2R, 7aS)-2-fluorotetrahydro-IH-pyrrolizin-7a(5H)-yl)methoxy)pyrido[4,3-d]pyrimidin-4-yl)piperazin-1-yl)-2-fluoro-3-(pyrimidin-2-yl)prop-2-en-1-one

7-(8-Ethynyl-6,7-difluoronaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (5 mg, 8.67 μmol) and (Z)-2-fluoro-3-(pyrimidin-2-yl)acrylic acid (4.4 mg, 0.026 mmol) were combined as solids and dissolved in DMF (400 μL). 1-methylimidazole (4.1 μL, 0.052 mmol) was added followed by chloro-N,N,N′,N′-tetramethylformamidinium hexafluorophosphate (7.3 mg, 0.026 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (4.0 mg, 5.4 μmol, 62% yield) as a brown solid. LCMS (ESI) m/z: [M+H]⁺ calcd for C₃₈H₃₂F₅N₈O₂ 727.3; found 727.3; ¹H NMR (500 MHz, DMSO-d₆) δ 9.12 (s, 1H), 8.89 (d, J=4.9 Hz, 2H), 8.30-8.23 (m, 1H), 8.16 (d, J=8.0 Hz, 1H), 7.75 (t, J=7.5 Hz, 1H), 7.66 (d, J=7.0 Hz, 1H), 7.44 (t, J=4.9 Hz, 1H), 6.63 (d, J=35.0 Hz, 1H), 5.28 (d, J=53.6 Hz, 1H), 4.25 (s, 1H), 4.20-3.85 (m, 10H), 3.18-3.02 (m, 3H), 2.88-2.78 (m, 1H), 2.17-1.98 (m, 3H), 1.94-1.69 (m, 3H).

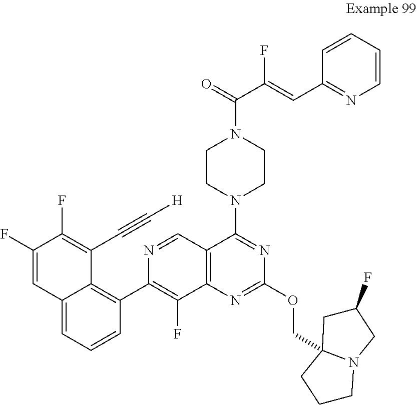

### (Z)-1-(4-(7-(8-ethynyl-6,7-difluoronaphthalen-1-yl)-8-fluoro-2-(((2R, 7aS)-2-fluorotetrahydro-IH-pyrrolizin-7a(5H)-yl)methoxy)pyrido[4,3-d]pyrimidin-4-yl)piperazin-1-yl)-2-fluoro-3-(pyridin-2-yl)prop-2-en-1-one

7-(8-Ethynyl-6,7-difluoronaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (5 mg, 8.67 μmol) and (Z)-2-fluoro-3-(pyridin-2-yl)acrylic acid (4.4 mg, 0.026 mmol) were combined as solids and dissolved in DMF (400 μL). 1-methylimidazole (4.1 μL, 0.052 mmol) was added followed by chloro-N,N,N′,N′-tetramethylformamidinium hexafluorophosphate (7.3 mg, 0.026 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 72% 5:95 MeCN:H₂O with 10 mM AA/28% 95:5 MeCN:H₂O with 10 mM AA→100% 95:5 MeCN:H₂O with 10 mM AA; λ=220 nm) to provide the desired product (4.5 mg, 6.1 μmol, 71% yield) as a brown solid. LCMS (ESI) m/z: [M+H]⁺ calcd for C₃₉H₃₃F₅N₇O₂ 726.3; found 726.3; ¹H NMR (500 MHz, DMSO-d₆) δ 9.13 (s, 1H), 8.65 (br s, 1H), 8.27 (br dd, J=10.6, 9.0 Hz, 1H), 8.17 (d, J=8.8 Hz, 1H), 7.89 (td, J=7.8, 1.7 Hz, 1H), 7.79 (d, J=7.9 Hz, 1H), 7.75 (t, J=7.7 Hz, 1H), 7.66 (br d, J=7.4 Hz, 1H), 7.38 (ddd, J=7.5, 4.8, 0.8 Hz, 1H), 6.66 (d, J=38.8 Hz, 1H), 5.29 (d, J=54.0 Hz, 1H), 4.26 (s, 1H), 4.20-3.88 (m, 10H), 3.15-3.04 (m, 3H), 2.89-2.80 (m, 1H), 2.22-1.98 (m, 3H), 1.90-1.74 (m, 3H).

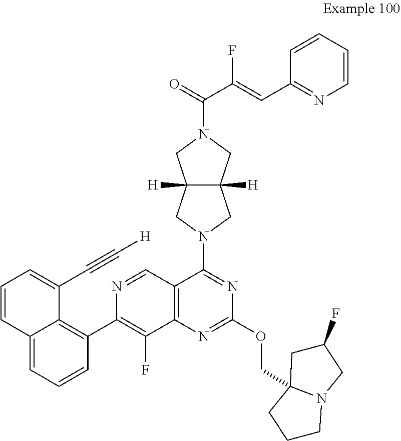

### (Z)-1-((3aR, 6aS)-5-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)hexahydropyrrolo[3,4-c]pyrrol-2(1H)-yl)-2-fluoro-3-(pyridin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-((3aR, 6aS)-hexahydropyrrolo[3,4-c]pyrrol-2(1H)-yl)pyrido[4,3-d]pyrimidine (5 mg, 8.8 μmol) and (Z)-2-fluoro-3-(pyridin-2-yl)acrylic acid (4.4 mg, 0.026 mmol) were combined as solids and dissolved in DMF (400 μL). 1-methylimidazole (4.1 μL, 0.052 mmol) was added followed by chloro-N,N,N′,N′-tetramethylformamidinium hexafluorophosphate (11 mg, 0.040 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (1.9 mg, 2.6 μmol, 30% yield) as a brown solid. LCMS (ESI) m/z: [M+H]⁺ calcd for C₄₁H₃₇F₃N₇O₂ 716.3; found 716.3; ¹H NMR (500 MHz, DMSO-d₆) δ 9.27 (br s, 1H), 8.63 (br d, J=4.9 Hz, 1H), 8.18-8.08 (m, 2H), 7.91-7.86 (m, 1H), 7.77 (d, J=8.2 Hz, 1H), 7.74-7.67 (m, 2H), 7.62-7.54 (m, 2H), 7.36 (dd, J=7.4, 5.5 Hz, 1H), 6.80 (d, J=37.7 Hz, 1H), 5.50-5.22 (m, 1H), 4.40-3.54 (m, 11H), 3.23-3.08 (m, 6H), 2.33-1.73 (m, 6H).

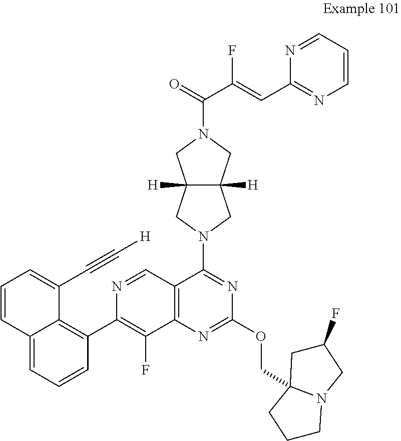

### (Z)-1-((3aR, 6aS)-5-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)hexahydropyrrolo[3,4-c]pyrrol-2(1H)-yl)-2-fluoro-3-(pyrimidin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-((3aR, 6aS)-hexahydropyrrolo[3,4-c]pyrrol-2(1H)-yl)pyrido[4,3-d]pyrimidine (5 mg, 8.8 μmol) were combined as solids and dissolved in DMF (400 μL). 1-methylimidazole (4.1 μL, 0.052 mmol) was added followed by chloro-N,N,N′,N′-tetramethylformamidinium hexafluorophosphate (11 mg, 0.040 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 85% 5:95 MeCN:H₂O with 10 mM AA/15% 95:5 MeCN:H₂O with 10 mM AA→100% 95:5 MeCN:H₂O with 10 mM AA; λ=220 nm) to provide the desired product (1.3 mg, 1.7 μmol, 20% yield) as a brown solid. LCMS (ESI) m/z: [M+H]⁺ calcd for C₄₀H₃₆F₃N₈O₂ 717.3; found 717.2; ¹H NMR (500 MHz, DMSO-d₆) δ 9.29 (br s, 1H), 8.87 (d, J=4.8 Hz, 2H), 8.18-8.10 (m, 2H), 7.75-7.67 (m, 2H), 7.57 (d, J=7.6 Hz, 2H), 7.43 (t, J=4.9 Hz, 1H), 6.77 (d, J=34.4 Hz, 1H), 5.40 (d, J=57.7 Hz, 1H), 4.53-3.63 (m, 10H), 3.25-3.03 (m, 7H), 2.41-1.84 (m, 6H).

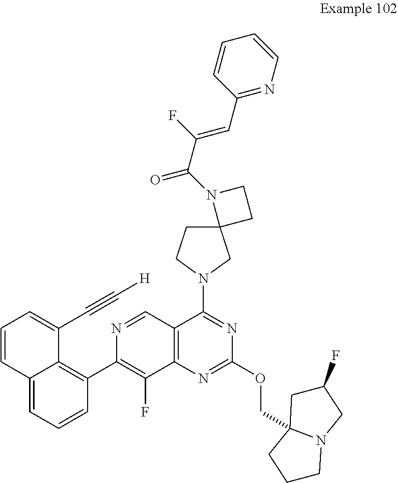

### (Z)-1-(6-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-1,6-diazaspiro[3.4]octan-1-yl)-2-fluoro-3-(pyridin-2-yl)prop-2-en-1-one

7-(8-ethynylnaphthalen-1-yl)-8-fluoro-2-(((2R, 7aS)-2-fluorotetrahydro-1H-pyrrolizin-7a(5H)-yl)methoxy)-4-(1,6-diazaspiro[3.4]octan-6-yl)pyrido[4,3-d]pyrimidine (Intermediate 58 Isomer 1, 5 mg, 8.8 μmol) and (Z)-2-fluoro-3-(pyridin-2-yl)acrylic acid (4.4 mg, 0.026 mmol) were combined as solids and dissolved in DMF (400 μL). 1-methylimidazole (4.1 μL, 0.052 mmol) was added followed by chloro-N,N,N′,N′-tetramethylformamidinium hexafluorophosphate (11 mg, 0.040 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (2.9 mg, 4.1 μmol, 46% yield) as a brown solid. LCMS (ESI) m/z: [M+H]⁺ calcd for C₄₁H₃₇F₃N₇O₂ 716.3; found 716.2; ¹H NMR (500 MHz, DMSO-d₆) δ 9.27-9.21 (m, 1H), 8.64 (br d, J=4.2 Hz, 1H), 8.14 (t, J=9.5 Hz, 2H), 7.90-7.83 (m, 1H), 7.77-7.67 (m, 3H), 7.62-7.54 (m, 2H), 7.36 (dd, J=7.3, 4.9 Hz, 1H), 6.81 (d, J=37.5 Hz, 1H), 5.28 (d, J=54.9 Hz, 1H), 4.82-4.55 (m, 1H), 4.46 (br d, J=5.5 Hz, 2H), 4.31-4.17 (m, 1H), 4.16-4.00 (m, 3H), 3.78-3.57 (m, 1H), 3.11-3.02 (m, 3H), 2.89-2.78 (m, 2H), 2.68-2.57 (m, 1H), 2.47-2.27 (m, 3H), 2.19-1.98 (m, 3H), 1.86-1.74 (m, 3H).

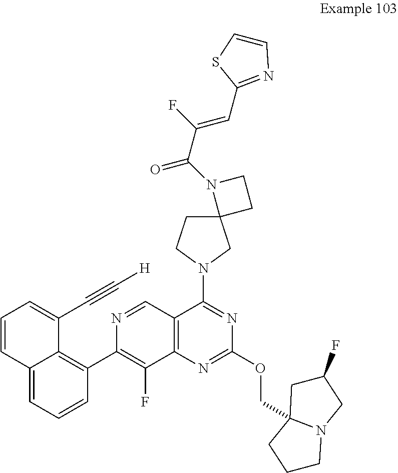

### (Z)-1-(6-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-1,6-diazaspiro[3.4]octan-1-yl)-2-fluoro-3-(thiazol-2-yl)prop-2-en-1-one

7-(8-ethynylnaphthalen-1-yl)-8-fluoro-2-(((2R, 7aS)-2-fluorotetrahydro-1H-pyrrolizin-7a(5H)-yl)methoxy)-4-(1,6-diazaspiro[3.4]octan-6-yl)pyrido[4,3-d]pyrimidine (Intermediate 58 Isomer 1, 5 mg, 8.8 μmol) and (Z)-2-fluoro-3-(thiazol-2-yl)acrylic acid (4.6 mg, 0.026 mmol) were combined as solids and dissolved in DMF (400 μL). 1-methylimidazole (4.1 μL, 0.052 mmol) was added followed by chloro-N,N,N′,N′-tetramethylformamidinium hexafluorophosphate (11 mg, 0.040 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide the desired product (4.0 mg, 5.5 μmol, 63% yield) as a brown solid. LCMS (ESI) m/z: [M+H]⁺ calcd for C₃₉H₃₅F₃N₇O₂ S 722.3; found 722.3; ¹H NMR (500 MHz, DMSO-d₆) δ 9.26-9.20 (m, 1H), 8.14 (t, J=9.4 Hz, 2H), 8.04-7.96 (m, 2H), 7.74-7.67 (m, 2H), 7.61-7.54 (m, 2H), 7.13 (d, J=36.3 Hz, 1H), 5.27 (d, J=53.9 Hz, 1H), 4.81-4.55 (m, 1H), 4.47 (br s, 2H), 4.34-3.98 (m, 4H), 3.79-3.57 (m, 1H), 3.13-2.99 (m, 3H), 2.90-2.80 (m, 2H), 2.69-2.59 (m, 1H), 2.49-2.42 (m, 2H), 2.38-2.29 (m, 1H), 2.20-1.96 (m, 3H), 1.88-1.71 (m, 3H).

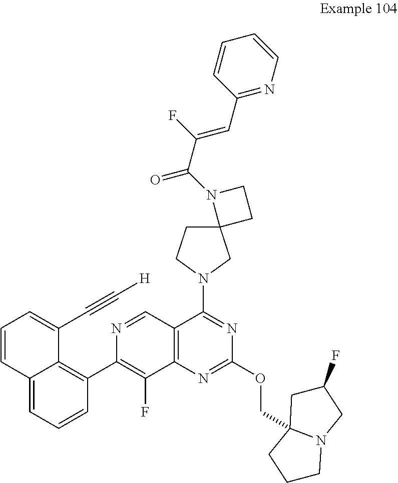

### (Z)-1-(6-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-1,6-diazaspiro[3.4]octan-1-yl)-2-fluoro-3-(pyridin-2-yl)prop-2-en-1-one

7-(8-ethynylnaphthalen-1-yl)-8-fluoro-2-(((2R, 7aS)-2-fluorotetrahydro-1H-pyrrolizin-7a(5H)-yl)methoxy)-4-(1,6-diazaspiro[3.4]octan-6-yl)pyrido[4,3-d]pyrimidine e (Intermediate 58 Isomer 2, 5 mg, 8.8 μmol) and (Z)-2-fluoro-3-(pyridin-2-yl)acrylic acid (4.4 mg, 0.026 mmol) were combined as solids and dissolved in DMF (400 μL). 1-methylimidazole (4.1 μL, 0.052 mmol) was added followed by chloro-N,N,N′,N′-tetramethylformamidinium hexafluorophosphate (11 mg, 0.040 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (5.7 mg, 7.8 μmol, 88% yield) as a brown solid. LCMS (ESI) m/z: [M+H]⁺ calcd for C₄₁H₃₇F₃N₇O₂ 716.3; found 716.2; ¹H NMR (500 MHz, DMSO-d₆) δ 9.31-9.14 (m, 1H), 8.63 (br d, J=4.8 Hz, 1H), 8.14 (t, J=9.3 Hz, 2H), 7.87 (t, J=7.9 Hz, 1H), 7.76-7.65 (m, 4H), 7.62-7.53 (m, 3H), 7.37 (dd, J=6.9, 4.9 Hz, 1H), 6.81 (d, J=37.4 Hz, 1H), 5.28 (d, J=52.9 Hz, 1H), 4.81-4.54 (m, 1H), 4.53-4.38 (m, 2H), 4.34-3.91 (m, 4H), 3.81-3.63 (m, 1H), 3.15-3.14 (m, 1H), 3.17-3.01 (m, 3H), 2.93-2.78 (m, 2H), 2.67-2.59 (m, 1H), 2.47-2.39 (m, 1H), 2.36-2.26 (m, 1H), 2.17-2.00 (m, 3H), 1.87-1.74 (m, 3H).

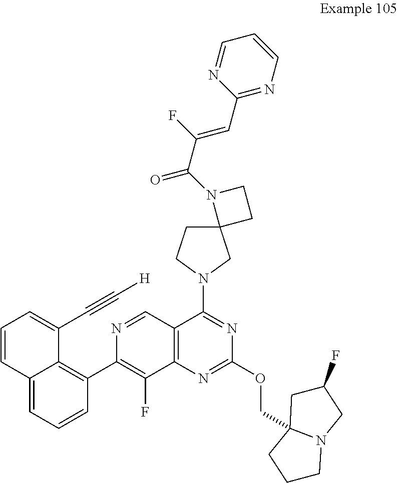

### (Z)-1-(6-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)-1,6-diazaspiro[3.4]octan-1-yl)-2-fluoro-3-(pyrimidin-2-yl)prop-2-en-1-one

7-(8-ethynylnaphthalen-1-yl)-8-fluoro-2-(((2R, 7aS)-2-fluorotetrahydro-1H-pyrrolizin-7a(5H)-yl)methoxy)-4-(1,6-diazaspiro[3.4]octan-6-yl)pyrido[4,3-d]pyrimidine (Intermediate 58 Isomer 2, 5 mg, 8.8 μmol) and (Z)-2-fluoro-3-(pyrimidin-2-yl)acrylic acid (4.4 mg, 0.026 mmol) were combined as solids and dissolved in DMF (400 μL). 1-methylimidazole (4.1 μL, 0.052 mmol) was added followed by chloro-N,N,N′,N′-tetramethylformamidinium hexafluorophosphate (11 mg, 0.040 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (2.6 mg, 3.6 μmol, 41% yield) as a brown solid. LCMS (ESI) m/z: [M+H]⁺ calcd for C₄₀H₃₆F₃N₈O₂ 717.3; found 717.2; ¹H NMR (500 MHz, DMSO-d₆) δ 9.27-9.21 (m, 1H), 8.87 (d, J=4.8 Hz, 2H), 8.14 (t, J=9.5 Hz, 2H), 7.75-7.67 (m, 2H), 7.63-7.53 (m, 2H), 7.43 (t, J=4.8 Hz, 1H), 6.78 (d, J=34.0 Hz, 1H), 5.27 (d, J=54.6 Hz, 1H), 4.81-4.52 (m, 1H), 4.45 (br d, J=2.9 Hz, 2H), 4.36-3.97 (m, 4H), 3.80-3.55 (m, 1H), 3.15-3.00 (m, 3H), 2.93-2.77 (m, 2H), 2.67-2.58 (m, 1H), 2.48-2.40 (m, 2H), 2.38-2.30 (m, 1H), 2.16-1.99 (m, 3H), 1.87-1.75 (m, 3H).

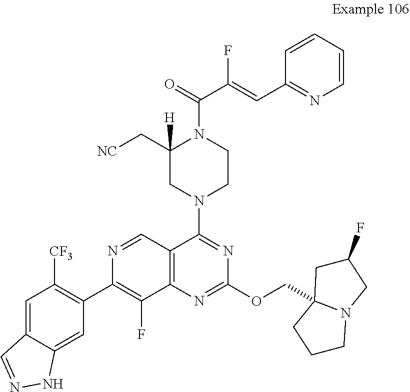

### 2-((S)-4-(8-fluoro-2-(((2R, 7aS)-2-fluorotetrahydro-1H-pyrrolizin-7a(5H)-yl)methoxy)-7-(5-methyl-1H-indazol-6-yl)pyrido[4,3-d]pyrimidin-4-yl)-1-((Z)-2-fluoro-3-(pyridin-2-yl)acryloyl)piperazin-2-yl)acetonitrile

2-((S)-4-(8-Fluoro-2-(((2R, 7aS)-2-fluorotetrahydro-1H-pyrrolizin-7a(5H)-yl)methoxy)-7-(5-(trifluoromethyl)-1H-indazol-6-yl)pyrido[4,3-d]pyrimidin-4-yl)piperazin-2-yl)acetonitrile (6.5 mg, 0.011 mmol) and (Z)-2-fluoro-3-(pyridin-2-yl)acrylic acid (5.3 mg, 0.032 mmol) were combined as solids and dissolved in DMF (400 L). 1-methylimidazole (5.1 μL, 0.064 mmol) was added followed by chloro-N,N,N′,N′-tetramethylformamidinium hexafluorophosphate (8.9 mg, 0.032 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (2.6 mg, 3.4 μmol, 32% yield) as an off-white solid. LCMS (ESI) m/z: [M+H]⁺ calcd for C₃₇H₃₃F₆N₁₀O₂ 763.3; found 763.3; ¹H NMR (500 MHz, DMSO-d₆) δ 13.67 (br s, 1H), 9.19 (s, 1H), 8.65 (br d, J=4.2 Hz, 1H), 8.45 (s, 1H), 8.38 (s, 1H), 7.90 (t, J=7.6 Hz, 1H), 7.80 (d, J=7.9 Hz, 1H), 7.72 (s, 1H), 7.39 (dd, J=7.1, 5.3 Hz, 1H), 6.67 (d, J=38.2 Hz, 1H), 5.27 (d, J=54.1 Hz, 1H), 4.97-4.81 (m, 1H), 4.53 (br d, J=12.9 Hz, 1H), 4.49-4.38 (m, 1H), 4.16 (d, J=10.6 Hz, 1H), 4.09 (br d, J=10.6 Hz, 1H), 4.27-4.03 (m, 1H), 3.87-3.71 (m, 2H), 3.15-3.01 (m, 6H), 2.86-2.79 (m, 1H), 2.16-1.95 (m, 3H), 1.88-1.74 (m, 3H).

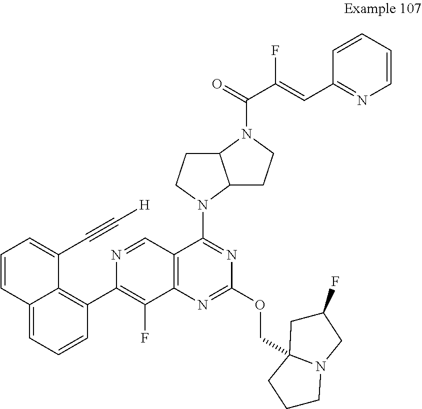

### (Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)hexahydropyrrolo[3,2-b]pyrrol-1(2H)-yl)-2-fluoro-3-(pyridin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-(hexahydropyrrolo[3,2-b]pyrrol-1(2H)-yl)pyrido[4,3-d]pyrimidine (Intermediate 59, isomer 1, 5 mg, 8.82 μmol) and (Z)-2-fluoro-3-(pyridin-2-yl)acrylic acid (4.4 mg, 0.026 mmol) were combined as solids and dissolved in DMF (400 L). 1-methylimidazole (4.1 μL, 0.052 mmol) was added followed by chloro-N,N,N′,N′-tetramethylformamidinium hexafluorophosphate (11 mg, 0.040 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (5.7 mg, 7.8 μmol, 88% yield) as a brown solid. LCMS (ESI) m/z: [M+H]⁺ calcd for C₄₁H₃₇F₃N₇O₂ 716.3; found 716.2; ¹H NMR (500 MHz, DMSO-d₆) δ 9.32-9.17 (m, 1H), 8.71-8.58 (m, 1H), 8.21-8.09 (m, 2H), 7.94-7.86 (m, 1H), 7.84-7.79 (m, 1H), 7.75-7.68 (m, 2H), 7.62-7.54 (m, 2H), 7.43-7.35 (m, 1H), 6.84 (d, J=36.6 Hz, 1H), 5.28 (d, J=54.0 Hz, 1H), 5.19-4.73 (m, 1H), 4.25-3.95 (m, 5H), 3.83-3.61 (m, 2H), 3.12-3.01 (m, 4H), 2.86-2.80 (m, 1H), 2.41-2.15 (m, 4H), 2.15-1.98 (m, 3H), 1.88-1.73 (m, 3H).

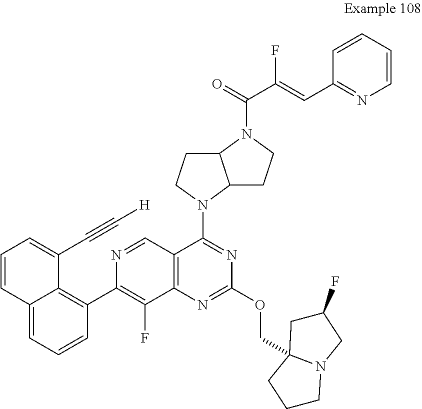

### (Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)hexahydropyrrolo[3,2-b]pyrrol-1(2H)-yl)-2-fluoro-3-(pyridin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-(hexahydropyrrolo[3,2-b]pyrrol-1(2H)-yl)pyrido[4,3-d]pyrimidine (Intermediate 59, isomer 2, 5 mg, 8.82 μmol) and (Z)-2-fluoro-3-(pyridin-2-yl)acrylic acid (4.4 mg, 0.026 mmol) were combined as solids and dissolved in DMF (400 L). 1-methylimidazole (4.1 μL, 0.052 mmol) was added followed by chloro-N,N,N′,N′-tetramethylformamidinium hexafluorophosphate (11 mg, 0.040 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (5.7 mg, 7.8 μmol, 88% yield) as a brown solid. LCMS (ESI) m/z: [M+H]⁺ calcd for C₄₁H₃₇F₃N₇O₂ 716.3; found 716.2; ¹H NMR (500 MHz, DMSO-d₆) δ 9.28-9.17 (m, 1H), 8.60 (s, 1H), 8.19-8.07 (m, 2H), 7.93-7.85 (m, 1H), 7.83-7.78 (m, 1H), 7.77-7.67 (m, 2H), 7.64-7.53 (m, 2H), 7.42-7.33 (m, 1H), 6.83 (d, J=37.8 Hz, 1H), 5.28 (d, J=56.5 Hz, 1H), 5.17-4.71 (m, 1H), 4.28-3.95 (m, 5H), 3.81-3.64 (m, 2H), 3.12-2.99 (m, 4H), 2.91-2.78 (m, 1H), 2.39-2.19 (m, 4H), 2.15-1.98 (m, 3H), 1.88-1.72 (m, 3H).

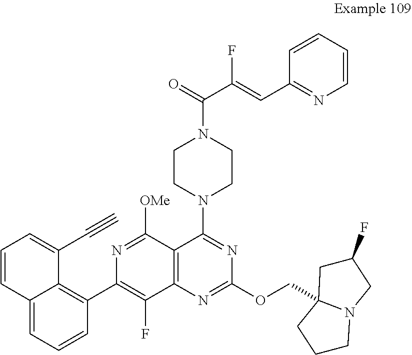 [SMILES: C#Cc1cccc2cccc(-c3nc(OC)c4c(N5CCN(C(=O)/C(F)=C/c6ccccn6)CC5)nc(OC[C@@]56CCCN5C[C@H](F)C6)nc4c3F)c12]

### (Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)-5-methoxypyrido[4,3-d]pyrimidin-4-yl)piperazin-1-yl)-2-fluoro-3-(pyridin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-5-methoxy-4-(piperazin-1-yl)pyrido[4,3-d]pyrimidine (22 mg, 0.039 mmol) and (Z)-2-fluoro-3-(pyridin-2-yl)acrylic acid (19 mg, 0.12 mmol) were combined as solids and dissolved in DMF (700 μL). 1-methylimidazole (18 μL, 0.23 mmol) was added followed by chloro-N,N,N′,N′-tetramethylformamidinium hexafluorophosphate (33 mg, 0.12 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 65% 5:95 MeCN:H₂O with 10 mM AA/35% 95:5 MeCN:H₂O with 10 mM AA→100% 95:5 MeCN:H₂O with 10 mM AA; λ=220 nm) to provide the desired product (18.8 mg, 0.026 mmol, 68% yield) as a brown solid. LCMS (ESI) m/z: [M+H]⁺ calcd for C₄₀H₃₇F₃N₇O₃ 720.3; found 720.2; ¹H NMR (500 MHz, DMSO-d₆) δ 8.68-8.61 (m, 1H), 8.17-8.11 (m, 2H), 7.93-7.86 (m, 1H), 7.81-7.76 (m, 1H), 7.76-7.72 (m, 1H), 7.71-7.68 (m, 1H), 7.67-7.64 (m, 1H), 7.59-7.54 (m, 1H), 7.41-7.35 (m, 1H), 6.61 (d, J=38.8 Hz, 1H), 5.32 (d, J=53.8 Hz, 1H), 4.29-4.02 (m, 2H), 3.92 (s, 3H), 3.88-3.73 (m, 9H), 3.28-3.04 (m, 3H), 2.96-2.82 (m, 1H), 2.27-1.97 (m, 3H), 1.91-1.71 (m, 3H).

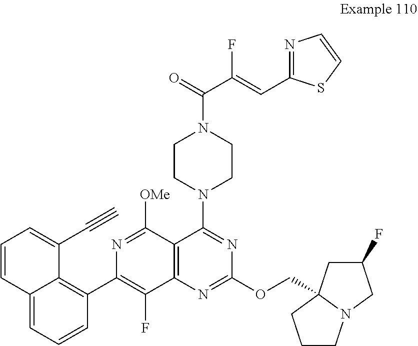 [SMILES: C#Cc1cccc2cccc(-c3nc(OC)c4c(N5CCN(C(=O)/C(F)=C/c6nccs6)CC5)nc(OC[C@@]56CCCN5C[C@H](F)C6)nc4c3F)c12]

### (Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)-5-methoxypyrido[4,3-d]pyrimidin-4-yl)piperazin-1-yl)-2-fluoro-3-(thiazol-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-5-methoxy-4-(piperazin-1-yl)pyrido[4,3-d]pyrimidine (22 mg, 0.039 mmol) and (Z)-2-fluoro-3-(thiazol-2-yl)acrylic acid (20 mg, 0.12 mmol) were combined as solids and dissolved in DMF (700 μL). 1-methylimidazole (18 μL, 0.23 mmol) was added followed by chloro-N,N,N′,N′-tetramethylformamidinium hexafluorophosphate (33 mg, 0.12 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 65% 5:95 MeCN:H₂O with 10 mM AA/35% 95:5 MeCN:H₂O with 10 mM AA→100% 95:5 MeCN:H₂O with 10 mM AA; λ=220 nm) to provide the desired product (16.7 mg, 0.023 mmol, 60% yield) as a brown solid. LCMS (ESI) m/z: [M+H]⁺ calcd for C₃₈H₃₅F₃N₇O₃S 726.2; found 726.1; ¹H NMR (500 MHz, DMSO-d₆) δ 8.16-8.11 (m, 2H), 8.03-7.97 (m, 2H), 7.75-7.72 (m, 1H), 7.72-7.67 (m, 1H), 7.67-7.64 (m, 1H), 7.57 (t, J=7.7 Hz, 1H), 7.07 (d, J=37.8 Hz, 1H), 5.30 (d, J=55.1 Hz, 1H), 4.28-4.01 (m, 2H), 3.91 (s, 3H), 3.90-3.71 (m, 9H), 3.23-3.01 (m, 3H), 2.90-2.78 (m, 1H), 2.21-1.99 (m, 3H), 1.91-1.71 (m, 3H).

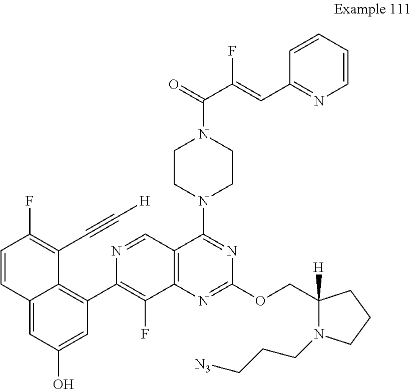 [SMILES: C#Cc1c(F)ccc2cc(O)cc(-c3ncc4c(N5CCN(C(=O)/C(F)=C/c6ccccn6)CC5)nc(OC[C@@H]5CCCN5CCCN=[N+]=[N-])nc4c3F)c12]

### (S,Z)-1-(4-(2-((1-(3-azidopropyl)pyrrolidin-2-yl)methoxy)-7-(8-ethynyl-7-fluoro-3-hydroxynaphthalen-1-yl)-8-fluoropyrido[4,3-d]pyrimidin-4-yl)piperazin-1-yl)-2-fluoro-3-(pyridin-2-yl)prop-2-en-1-one

(S)-4-(2-((1-(3-Azidopropyl)pyrrolidin-2-yl)methoxy)-8-fluoro-4-(piperazin-1-yl)pyrido[4,3-d]pyrimidin-7-yl)-5-ethynyl-6-fluoronaphthalen-2-ol (320 mg, 0.534 mmol) and (Z)-2-fluoro-3-(pyridin-2-yl)acrylic acid (178 mg, 1.07 mmol) were combined as solids and dissolved in DMF (5.3 mL). 1-methylimidazole (210 μL, 2.67 mmol) was added followed by chloro-N,N,N′,N′-tetramethylformamidinium hexafluorophosphate (300 mg, 1.07 mmol). The reaction mixture was stirred for 5 min. Ammonium hydroxide solution (100 μL) and sodium hydroxide solution (100 μL) were added, and the mixture was stirred for 5 min. The solution was directly purified by reverse phase HPLC (column: Xbridge C18, 19 mm×200 mm, 5 m particles; flow rate: 20 mL/min; column temperature: 25° C.; gradient: 100% 5:95 MeCN:H₂O with 10 mM AA/0% 95:5 MeCN:H₂O with 10 mM AA→100% 95:5 MeCN:H₂O with 10 mM AA; λ=220 nm) to provide the desired product (160 mg, 0.21 mmol, 40% yield) as a brown solid. LCMS (ESI) m/z: [M+H]⁺ calcd for C₃₉H₃₆F₃N₁₀O₃ 749.3; found 749.3.

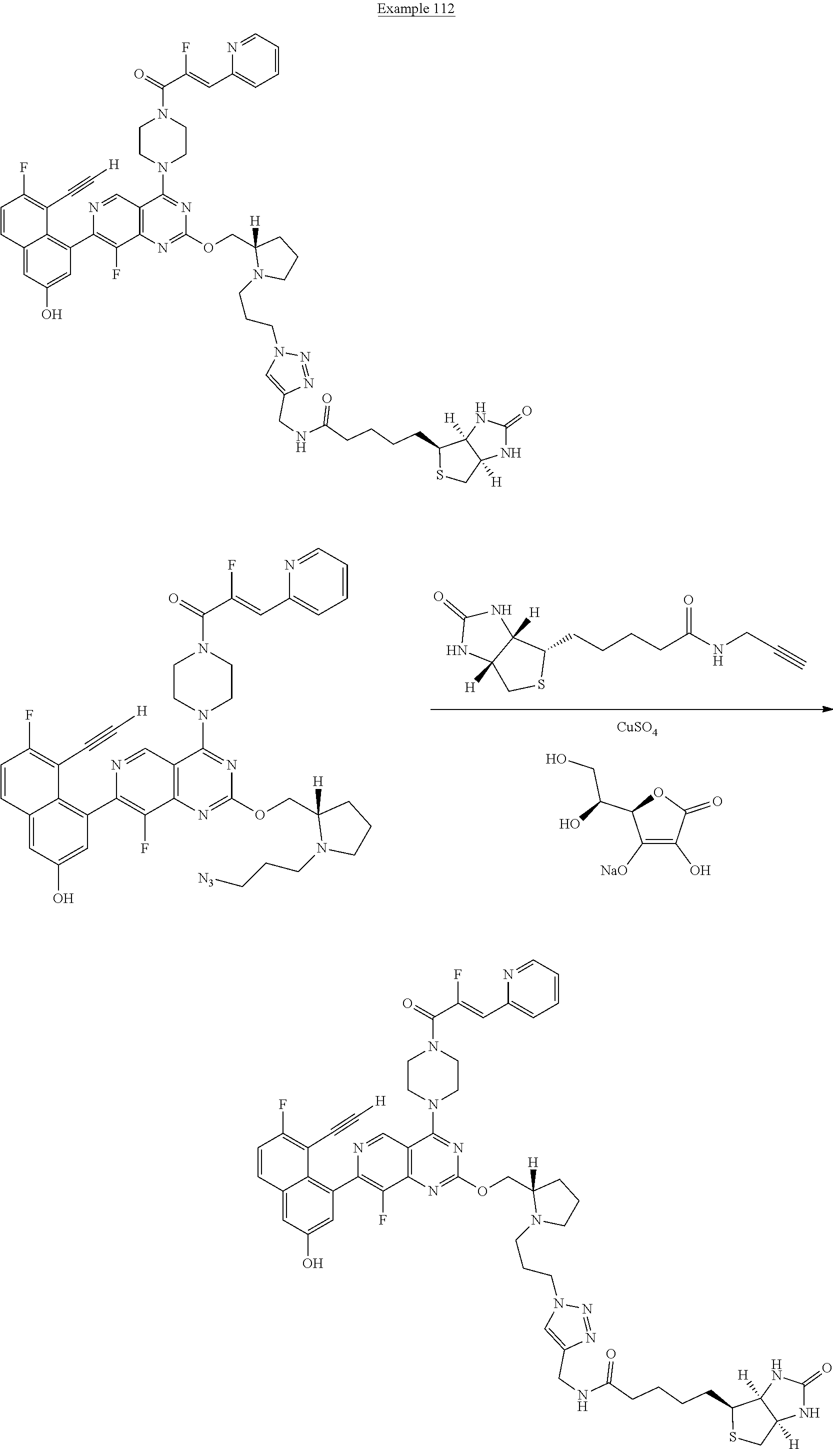

### N-((1-(3-((S)-2-(((7-(8-ethynyl-7-fluoro-3-hydroxynaphthalen-1-yl)-8-fluoro-4-(4-((Z)-2-fluoro-3-(pyridin-2-yl)acryloyl)piperazin-1-yl)pyrido[4,3-d]pyrimidin-2-yl)oxy)methyl)pyrrolidin-1-yl)propyl)-1H-1,2,3-triazol-4-yl)methyl)-5-((3aS,4S,6aR)-2-oxohexahydro-1H-thieno[3,4-d]imidazol-4-yl)pentanamide

(S,Z)-1-(4-(2-((1-(3-Azidopropyl)pyrrolidin-2-yl)methoxy)-7-(8-ethynyl-7-fluoro-3-hydroxynaphthalen-1-yl)-8-fluoropyrido[4,3-d]pyrimidin-4-yl)piperazin-1-yl)-2-fluoro-3-(pyridin-2-yl)prop-2-en-1-one (30 mg, 0.040 mmol) and 5-((3aS,4S,6aR)-2-oxohexahydro-1H-thieno[3,4-d]imidazol-4-yl)-N-(prop-2-yn-1-yl)pentanamide (33.8 mg, 0.120 mmol) were combined and dissolved in MeOH (400 μL). Copper(II) sulfate solution (50 mg/mL in water, 40 μL, 8.0 μmol) was added dropwise followed by sodium ascorbate (12 mg, 0.06 mmol). The reaction mixture was stirred at room temperature for 30 min. Saturated aqueous ammonium hydroxide solution (100 μL) was added, and the mixture was stirred for 10 min. The solution was directly purified by reverse phase HPLC (column: Xbridge C18, 19 mm×200 mm, 5 m particles; flow rate: 20 mL/min; column temperature: 25° C.; gradient: 86% 5:95 MeCN:H₂O with 10 mM AA/14% 95:5 MeCN:H₂O with 10 mM AA→100% 95:5 MeCN:H₂O with 10 mM AA; λ=220 nm) to provide the desired product (14 mg, 0.013 mmol, 33% yield) as a brown solid. LCMS (ESI) m/z: [M+H]⁺ calcd for C₅₂H₅₅F₃N₁₃O₅S 1030.4; found 1030.4.

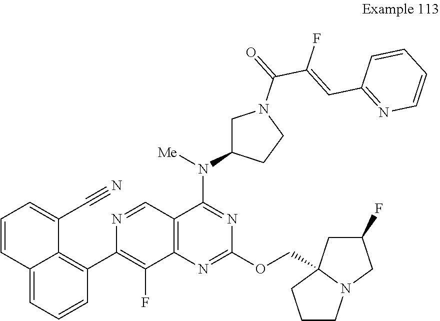 [SMILES: CN(c1nc(OC[C@@]23CCCN2C[C@H](F)C3)nc2c(F)c(-c3cccc4cccc(C#N)c34)ncc12)[C@@H]1CCN(C(=O)/C(F)=C/c2ccccn2)C1]

### 8-(8-fluoro-4-(((R)-1-((Z)-2-fluoro-3-(pyridin-2-yl)acryloyl)pyrrolidin-3-yl)(methyl)amino)-2-(((2R, 7aS)-2-fluorotetrahydro-1H-pyrrolizin-7a(5H)-yl)methoxy)pyrido[4,3-d]pyrimidin-7-yl)-1-naphthonitrile

To a stirred solution of 8-(8-fluoro-2-(((2R, 7aS)-2-fluorotetrahydro-1H-pyrrolizin-7a(5H)-yl)methoxy)-4-(methyl((R)-pyrrolidin-3-yl)amino)pyrido[4,3-d]pyrimidin-7-yl)-1-naphthonitrile (8 mg, 0.014 mmol), (Z)-2-fluoro-3-(pyridin-2-yl)acrylic acid (4.81 mg, 0.029 mmol), and 1-methylimidazole (0.017 mL, 0.216 mmol) in DMF (1 mL) was added chloro-N,N,N′,N′-tetramethylformamidinium hexafluorophosphate (8.08 mg, 0.029 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 82% 5:95 MeCN:H₂O with 10 mM AA/18% 95:5 MeCN:H₂O with 10 mM AA→100% 95:5 MeCN:H₂O with 10 mM AA; λ=220 nm) to provide the desired product (4.5 mg, 0.006 mmol, 43% yield) as a brown solid. LC/MS (ESI) m/z: [M+H]⁺ calcd for C₃₉H₃₅F₃N₈O₂ 705.3; found 705.3; ¹H NMR (500 MHz, DMSO-d₆) δ 9.29 (s, 1H), 8.65 (s, 1H), 8.47 (d, J=8.3 Hz, 1H), 8.30 (br d, J=7.8 Hz, 1H), 8.15 (br d, J=7.2 Hz, 1H), 7.98-7.65 (m, 5H), 7.38 (br d, J=4.7 Hz, 1H), 6.80 (br d, J=37.6 Hz, 1H), 5.39-5.14 (m, 3H), 4.33-3.74 (m, 5H), 3.70-3.52 (m, 1H), 3.47 (s, 3H), 3.13-2.72 (m, 3H), 2.45-2.23 (m, 1H), 2.20-1.66 (m, 7H).

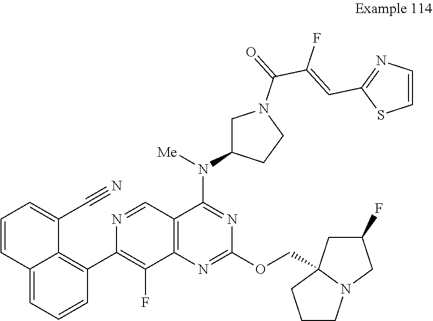 [SMILES: CN(c1nc(OC[C@@]23CCCN2C[C@H](F)C3)nc2c(F)c(-c3cccc4cccc(C#N)c34)ncc12)[C@@H]1CCN(C(=O)/C(F)=C/c2nccs2)C1]

### 8-(8-fluoro-4-(((R)-1-((Z)-2-fluoro-3-(thiazol-2-yl)acryloyl)pyrrolidin-3-yl)(methyl)amino)-2-(((2R, 7aS)-2-fluorotetrahydro-1H-pyrrolizin-7a(5H)-yl)methoxy)pyrido[4,3-d]pyrimidin-7-yl)-1-naphthonitrile

To a stirred solution of 8-(8-fluoro-2-(((2R, 7aS)-2-fluorotetrahydro-1H-pyrrolizin-7a(5H)-yl)methoxy)-4-(methyl((R)-pyrrolidin-3-yl)amino)pyrido[4,3-d]pyrimidin-7-yl)-1-naphthonitrile (8 mg, 0.014 mmol), (Z)-2-fluoro-3-(thiazol-2-yl)acrylic acid (4.99 mg, 0.029 mmol), and 1-methylimidazole (0.017 mL, 0.216 mmol) in DMF (1 mL) was added chloro-N,N,N′,N′-tetramethylformamidinium hexafluorophosphate (8.08 mg, 0.029 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 79% 5:95 MeCN:H₂O with 10 mM AA/21% 95:5 MeCN:H₂O with 10 mM AA→100% 95:5 MeCN:H₂O with 10 mM AA; λ=220 nm) to provide the desired product (6.6 mg, 0.009 mmol, 62% yield) as a brown solid. LC/MS (ESI) m/z: [M+H]⁺ calcd for C₃₇H₃₃F₃N₈O₂ S 711.2; found 711.3; ¹H NMR (500 MHz, DMSO-d₆) δ 9.29 (s, 1H), 8.47 (d, J=8.3 Hz, 1H), 8.30 (d, J=7.9 Hz, 1H), 8.15 (br d, J=7.1 Hz, 1H), 8.01 (br d, J=9.1 Hz, 2H), 7.88-7.78 (m, 2H), 7.75 (t, J=7.8 Hz, 1H), 7.21 (br d, J=36.6 Hz, 1H), 5.50-5.08 (m, 3H), 4.35-4.24 (m, 1H), 4.22-4.05 (m, 2H), 4.03-3.76 (m, 2H), 3.70-3.52 (m, 1H), 3.47 (s, 3H), 3.12-2.96 (m, 2H), 2.87-2.73 (m, 1H), 2.45-2.21 (m, 2H), 2.17-1.94 (m, 3H), 1.88-1.66 (m, 3H).

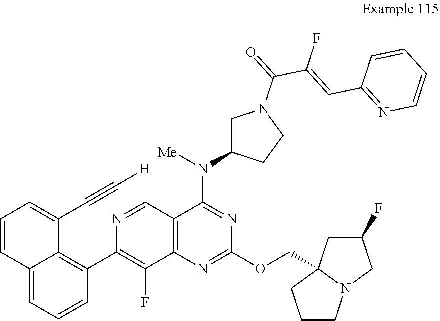 [SMILES: C#Cc1cccc2cccc(-c3ncc4c(N(C)[C@@H]5CCN(C(=O)/C(F)=C/c6ccccn6)C5)nc(OC[C@@]56CCCN5C[C@H](F)C6)nc4c3F)c12]

### (Z)-1-((R)-3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoro-3-(pyridin-2-yl)prop-2-en-1-one

To a stirred solution of 7-(8-ethynylnaphthalen-1-yl)-8-fluoro-2-(((2R, 7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (19 mg, 0.034 mmol), (Z)-2-fluoro-3-(pyridin-2-yl)acrylic acid (11.45 mg, 0.069 mmol), and 1-methylimidazole (0.041 mL, 0.514 mmol) in DMF (1 mL) was added chloro-N,N,N′,N′-tetramethylformamidinium hexafluorophosphate (19.22 mg, 0.069 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (8.8 mg, 0.013 mmol, 37% yield) as a brown solid. LC/MS (ESI) m/z: [M+H]⁺ calcd for C₄₀H₃₆F₃N₇O₂ 704.3; found 704.4; ¹H NMR (500 MHz, DMSO-d₆) δ 9.11 (br d, J=7.1 Hz, 1H), 8.56 (s, 1H), 8.06 (dd, J=11.7, 8.2 Hz, 2H), 7.85-7.76 (m, 1H), 7.73-7.66 (m, 1H), 7.65-7.58 (m, 2H), 7.56-7.44 (m, 2H), 7.33-7.24 (m, 1H), 6.73 (m, d, J=37.7 Hz, 1H), 5.32-4.98 (m, 2H), 4.37-3.39 (m, 7H), 3.37 (s, 3H), 3.35-3.23 (m, 1H), 3.04-2.51 (m, 3H), 2.36-2.17 (m, 2H), 2.12-1.85 (m, 3H), 1.81-1.49 (m, 3H).

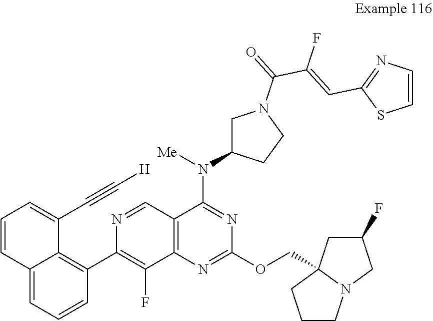 [SMILES: C#Cc1cccc2cccc(-c3ncc4c(N(C)[C@@H]5CCN(C(=O)/C(F)=C/c6nccs6)C5)nc(OC[C@@]56CCCN5C[C@H](F)C6)nc4c3F)c12]

### (Z)-1-((R)-3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoro-3-(thiazol-2-yl)prop-2-en-1-one

To a stirred solution of 7-(8-ethynylnaphthalen-1-yl)-8-fluoro-2-(((2R, 7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (19 mg, 0.034 mmol), (Z)-2-fluoro-3-(thiazol-2-yl)acrylic acid (11.9 mg, 0.069 mmol), and 1-methylimidazole (0.041 mL, 0.514 mmol) in DMF (1 mL) was added chloro-N,N,N′,N′-tetramethylformamidinium hexafluorophosphate (19.2 mg, 0.069 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (14 mg, 0.020 mmol, 58% yield) as a brown solid. LC/MS (ESI) m/z: [M+H]⁺ calcd for C₃₈H₃₄F₃N₇O₂ S 710.3; found 710.3; ¹H NMR (500 MHz, DMSO-d₆) δ 9.19 (d, J=7.0 Hz, 1H), 8.15 (dd, J=11.6, 8.2 Hz, 2H), 8.01 (br d, J=11.0 Hz, 2H), 7.76-7.66 (m, 2H), 7.65-7.50 (m, 2H), 7.21 (d, J=36.7 Hz 1H), 5.42-5.10 (m, 2H), 4.29 (br d, J=6.2 Hz, 1H), 4.19-3.50 (m, 6H), 3.46 (s, 3H), 3.43-3.30 (m, 1H), 3.20-2.91 (m, 2H), 2.79 (br d, J=5.5 Hz, 1H), 2.46-2.22 (m, 2H), 2.20-1.93 (m, 3H), 1.89-1.60 (m, 3H).

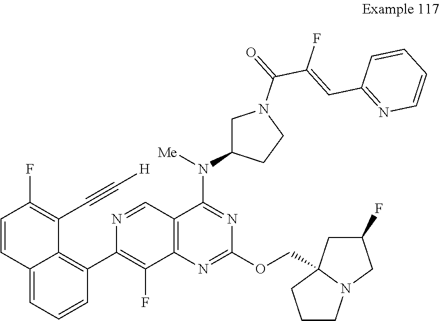 [SMILES: C#Cc1c(F)ccc2cccc(-c3ncc4c(N(C)[C@@H]5CCN(C(=O)/C(F)=C/c6ccccn6)C5)nc(OC[C@@]56CCCN5C[C@H](F)C6)nc4c3F)c12]

### (Z)-1-((R)-3-((7-(8-ethynyl-7-fluoronaphthalen-1-yl)-8-fluoro-2-(((2R, 7aS)-2-fluorotetrahydro-IH-pyrrolizin-7a(5H)-yl)methoxy)pyrido[4,3-d]pyrimidin-4-yl)(methyl)amino)pyrrolidin-1-yl)-2-fluoro-3-(pyridin-2-yl)prop-2-en-1-one

To a stirred solution of 7-(8-ethynyl-7-fluoronaphthalen-1-yl)-8-fluoro-2-(((2R, 7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (9 mg, 0.016 mmol), (Z)-2-fluoro-3-(pyridin-2-yl)acrylic acid (5.3 mg, 0.031 mmol), and 1-methylimidazole (0.019 mL, 0.236 mmol) in DMF (0.5 mL) was added chloro-N,N,N′,N′-tetramethylformamidinium hexafluorophosphate (8.8 mg, 0.031 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 76% 5:95 MeCN:H₂O with 10 mM AA/24% 95:5 MeCN:H₂O with 10 mM AA→100% 95:5 MeCN:H₂O with 10 mM AA; λ=220 nm) to provide the desired product (7.4 mg, 0.010 mmol, 65% yield) as a brown solid. LC/MS (ESI) m/z: [M+H]⁺ calcd for C₄₀H₃₅F₄N₇O₂ 722.3; found 722.3; ¹H NMR (500 MHz, DMSO-d₆) δ 9.20 (br d, J=7.0 Hz, 1H), 8.65 (br t, J=6.2 Hz, 1H), 8.27-8.12 (m, 2H), 7.96-7.84 (m, 1H), 7.84-7.75 (m, 1H), 7.73-7.65 (m, 2H), 7.61 (t, J=9.0 Hz, 1H), 7.38 (s, 1H), 6.82 (d, J=38.2 Hz, 1H), 5.38-5.11 (m, 2H), 4.28 (br d, J=7.7 Hz, 1H), 4.19-3.51 (m, 6H), 3.47 (s, 3H), 3.41-4.30 (m, 1H), 3.14-2.71 (m, 3H), 2.44-2.23 (m, 2H), 2.20-1.93 (m, 3H), 1.88-1.61 (m, 3H).

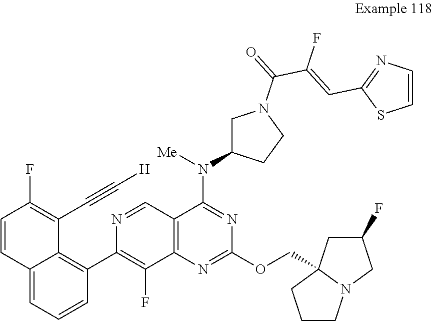 [SMILES: C#Cc1c(F)ccc2cccc(-c3ncc4c(N(C)[C@@H]5CCN(C(=O)/C(F)=C/c6nccs6)C5)nc(OC[C@@]56CCCN5C[C@H](F)C6)nc4c3F)c12]

### (Z)-1-((R)-3-((7-(8-ethynyl-7-fluoronaphthalen-1-yl)-8-fluoro-2-(((2R, 7aS)-2-fluorotetrahydro-IH-pyrrolizin-7a(5H)-yl)methoxy)pyrido[4,3-d]pyrimidin-4-yl)(methyl)amino)pyrrolidin-1-yl)-2-fluoro-3-(thiazol-2-yl)prop-2-en-1-one

To a stirred solution of 7-(8-ethynyl-7-fluoronaphthalen-1-yl)-8-fluoro-2-(((2R, 7aS)-2-fluorotetrahydro-1H-pyrrolizin-7a(5H)-yl)methoxy)-N-methyl-N—((R)-pyrrolidin-3-yl)pyrido[4,3-d]pyrimidin-4-amine (9 mg, 0.016 mmol), (Z)-2-fluoro-3-(thiazol-2-yl)acrylic acid (5.4 mg, 0.031 mmol), and 1-methylimidazole (0.019 mL, 0.236 mmol) in DMF (0.5 mL) was added chloro-N,N,N′,N′-tetramethylformamidinium hexafluorophosphate (8.8 mg, 0.031 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (6.2 mg, 0.009 mmol, 54% yield) as a brown solid. LC/MS (ESI) m/z: [M+H]⁺ calcd for C₃₈H₃₃F₄N₇O₂ S 728.2; found 728.2; ¹H NMR (500 MHz, DMSO-d₆) δ 9.23 (br d, J=6.7 Hz, 1H), 8.29-8.15 (m, 2H), 8.01 (br d, J=10.8 Hz, 2H), 7.76-7.65 (m, 2H), 7.61 (t, J=9.0 Hz, 1H), 7.21 (d, J=36.6 Hz, 1H), 5.57-5.22 (m, 2H), 4.52-4.21 (m, 2H), 4.17-3.52 (m, 5H), 3.49 (s, 3H), 3.38 (br d, J=16.3 Hz, 1H), 3.20-2.67 (m, 3H), 2.48-1.68 (m, 8H).

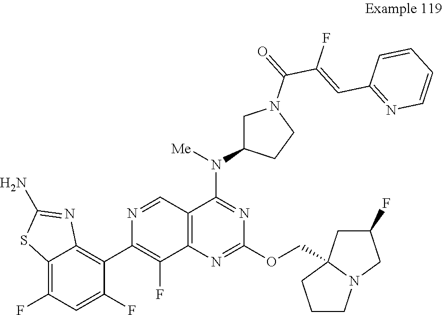 [SMILES: CN(c1nc(OC[C@@]23CCCN2C[C@H](F)C3)nc2c(F)c(-c3c(F)cc(F)c4sc(N)nc34)ncc12)[C@@H]1CCN(C(=O)/C(F)=C/c2ccccn2)C1]

### (Z)-1-((3R)-3-((7-(2-amino-5,7-difluorobenzo[d]thiazol-4-yl)-8-fluoro-2-(((7aS)-2-fluorotetrahydro-1H-pyrrolizin-7a(5H)-yl)methoxy)pyrido[4,3-d]pyrimidin-4-yl)(methyl)amino)pyrrolidin-1-yl)-2-fluoro-3-(pyridin-2-yl)prop-2-en-1-one

To a stirred solution of tert-butyl (5,7-difluoro-4-(8-fluoro-2-(((7aS)-2-fluorotetrahydro-1H-pyrrolizin-7a(5H)-yl)methoxy)-4-(methyl((R)-pyrrolidin-3-yl)amino)pyrido[4,3-d]pyrimidin-7-yl)benzo[d]thiazol-2-yl)carbamate (14 mg, 0.020 mmol), (Z)-2-fluoro-3-(pyridin-2-yl)acrylic acid (6.8 mg, 0.041 mmol), and 1-methylimidazole (0.024 mL, 0.305 mmol) in DMF (0.5 mL) was added chloro-N,N,N′,N′-tetramethylformamidinium hexafluorophosphate (11.4 mg, 0.041 mmol). The mixture was stirred for 1 h. The solvent was concentrated. The resulting dark residue and trifluoroacetic acid (0.1 mL) were stirred in CH₂C₂ (0.5 mL) for 30 min. The solvent was concentrated, dissolved in acetonitrile, made basic with 1-methylimidazole (0.014 mL, 0.180 mmol) and purified by reverse phase HPLC (column: Xbridge C18, 19 mm×200 mm, 5 m particles; flow rate: 20 mL/min; column temperature: 25° C.; gradient: 81% 5:95 MeCN:H₂O with 10 mM AA/19% 95:5 MeCN:H₂O with 10 mM AA→100% 95:5 MeCN:H₂O with 10 mM AA; λ=220 nm) to provide the desired product (6.4 mg, 0.009 mmol, 49% yield) as a brown solid. LC/MS (ESI) m/z: [M+H]⁺ calcd for C₃₅H₃₂F₈N₉O₂ S 738.2; found 738.3; ¹H NMR (500 MHz, DMSO-d₆) δ 9.21 (s, 1H), 8.60 (s, 1H), 8.02 (s, 2H), 7.88-7.79 (m, 1H), 7.74 (br t, J=7.9 Hz, 1H), 7.37-7.27 (m, 1H), 7.10 (br t, J=9.9 Hz, 1H), 6.77 (d, J=35.2 Hz, 1H), 5.35-4.97 (m, 2H), 4.30-3.90 (m, 4H), 3.82-3.44 (m, 2H), 3.38 (s, 1H), 3.33 (s, 3H), 3.08-2.89 (m, 2H), 2.83-2.62 (m, 1H), 2.38-2.13 (m, 2H), 2.13-1.58 (m, 6H).

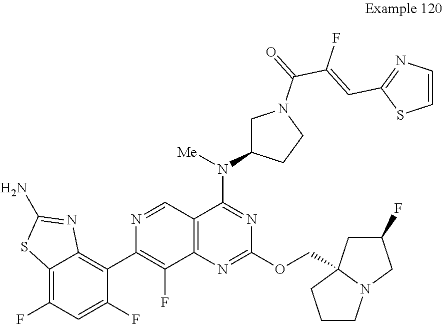 [SMILES: CN(c1nc(OC[C@@]23CCCN2C[C@H](F)C3)nc2c(F)c(-c3c(F)cc(F)c4sc(N)nc34)ncc12)[C@@H]1CCN(C(=O)/C(F)=C/c2nccs2)C1]

### tert-butyl (5,7-difluoro-4-(8-fluoro-4-(((R)-1-((Z)-2-fluoro-3-(thiazol-2-yl)acryloyl)pyrrolidin-3-yl)(methyl)amino)-2-(((7aS)-2-fluorotetrahydro-1H-pyrrolizin-7a(5H)-yl)methoxy)pyrido[4,3-d]pyrimidin-7-yl)benzo[d]thiazol-2-yl)carbamate

To a stirred solution of tert-butyl (5,7-difluoro-4-(8-fluoro-2-(((7aS)-2-fluorotetrahydro-1H-pyrrolizin-7a(5H)-yl)methoxy)-4-(methyl((R)-pyrrolidin-3-yl)amino)pyrido[4,3-d]pyrimidin-7-yl)benzo[d]thiazol-2-yl)carbamate (14 mg, 0.020 mmol), (Z)-2-fluoro-3-(thiazol-2-yl)acrylic acid (7.0 mg, 0.041 mmol), and 1-methylimidazole (0.024 mL, 0.305 mmol) in DMF (0.5 mL) was added chloro-N,N,N′,N′-tetramethylformamidinium hexafluorophosphate (11.4 mg, 0.041 mmol). The mixture was stirred for 1 h. The solvent was concentrated. The resulting dark residue and trifluoroacetic acid (0.1 mL) were stirred in CH₂C₂ (0.5 mL) for 30 min. The solvent was concentrated, dissolved in acetonitrile, made basic with 1-methylimidazole (0.014 mL, 0.180 mmol) and purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (7.4 mg, 0.010 mmol, 55% yield) as a brown solid. LC/MS (ESI) m/z: [M+H]⁺ calcd for C₃₃H₃₀F₅N₉O₂ S₂ 744.2; found 744.2; ¹H NMR (500 MHz, DMSO-d₆) δ 9.26 (s, 1H), 8.06 (s, 2H), 8.01 (br d, J=10.2 Hz, 2H), 7.20 (d, J=36.7 Hz, 1H), 7.18-7.09 (m, 1H), 5.47-5.11 (m, 2H), 4.47-3.92 (m, 5H), 3.87-3.51 (m, 2H), 3.38 (s, 3H), 3.12-2.73 (m, 3H), 2.42-2.21 (m, 2H), 2.14-1.61 (m, 6H).

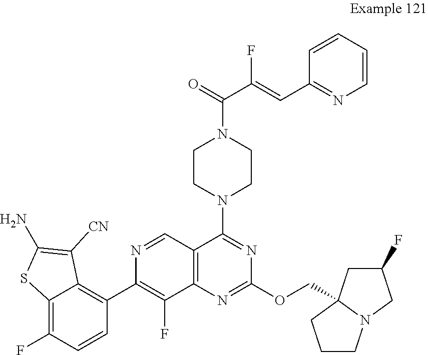 [SMILES: N#Cc1c(N)sc2c(F)ccc(-c3ncc4c(N5CCN(C(=O)/C(F)=C/c6ccccn6)CC5)nc(OC[C@@]56CCCN5C[C@H](F)C6)nc4c3F)c12]

### 2-amino-7-fluoro-4-(8-fluoro-4-(4-((Z)-2-fluoro-3-(pyridin-2-yl)acryloyl)piperazin-1-yl)-2-(((7aS)-2-fluorotetrahydro-1H-pyrrolizin-7a(5H)-yl)methoxy)pyrido[4,3-d]pyrimidin-7-yl)benzo[b]thiophene-3-carbonitrile

To a stirred solution of 2-amino-7-fluoro-4-(8-fluoro-2-(((7aS)-2-fluorotetrahydro-1H-pyrrolizin-7a(5H)-yl)methoxy)-4-(piperazin-1-yl)pyrido[4,3-d]pyrimidin-7-yl)benzo[b]thiophene-3-carbonitrile (9 mg, 0.016 mmol), (Z)-2-fluoro-3-(pyridin-2-yl)acrylic acid (5.2 mg, 0.031 mmol), and 1-methylimidazole (0.019 mL, 0.233 mmol) in acetonitrile (1 mL) was added chloro-N,N,N′,N′-tetramethylformamidinium hexafluorophosphate (8.7 mg, 0.031 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 80% 5:95 MeCN:H₂O with 10 mM AA/20% 95:5 MeCN:H₂O with 10 mM AA→100% 95:5 MeCN:H₂O with 10 mM AA; λ=220 nm) to provide the desired product (4.7 mg, 0.006 mmol, 40% yield) as a brown solid. LC/MS (ESI) m/z: [M+H]⁺ calcd for C₃₆H₃₁F₄N₉O₂ S 730.2; found 730.2; H NMR (500 MHz, DMSO-d₆) δ 9.18 (s, 1H), 8.65 (br d, J=4.8 Hz, 1H), 8.09 (bs, 2H), 7.94-7.85 (m, 1H), 7.79 (d, J=7.9 Hz, 1H), 7.44-7.27 (m, 2H), 7.16 (t, J=8.9 Hz, 1H), 6.67 (d, J=38.6 Hz, 1H), 5.42-5.08 (m, 2H), 4.14 (s, 6H), 3.91 (s, 4H), 3.43-3.01 (m, 2H), 2.92-2.76 (m, 1H), 2.23-1.97 (m, 3H), 1.90-1.68 (m, 3H).

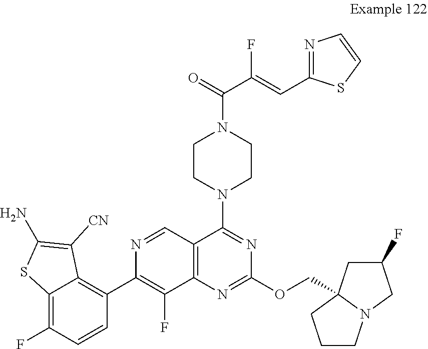 [SMILES: N#Cc1c(N)sc2c(F)ccc(-c3ncc4c(N5CCN(C(=O)/C(F)=C/c6nccs6)CC5)nc(OC[C@@]56CCCN5C[C@H](F)C6)nc4c3F)c12]

### 2-amino-7-fluoro-4-(8-fluoro-4-(4-((Z)-2-fluoro-3-(thiazol-2-yl)acryloyl)piperazin-1-yl)-2-(((7aS)-2-fluorotetrahydro-1H-pyrrolizin-7a(5H)-yl)methoxy)pyrido[4,3-d]pyrimidin-7-yl)benzo[b]thiophene-3-carbonitrile

To a stirred solution of 2-amino-7-fluoro-4-(8-fluoro-2-(((7aS)-2-fluorotetrahydro-1H-pyrrolizin-7a(5H)-yl)methoxy)-4-(piperazin-1-yl)pyrido[4,3-d]pyrimidin-7-yl)benzo[b]thiophene-3-carbonitrile (10 mg, 0.017 mmol), (Z)-2-fluoro-3-(thiazol-2-yl)acrylic acid (6.0 mg, 0.034 mmol), and 1-methylimidazole (0.021 mL, 0.258 mmol) in DMF (1 mL) was added chloro-N,N,N′,N′-tetramethylformamidinium hexafluorophosphate (9.7 mg, 0.034 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 82% 5:95 MeCN:H₂O with 10 mM AA/18% 95:5 MeCN:H₂O with 10 mM AA→100% 95:5 MeCN:H₂O with 10 mM AA; λ=220 nm) to provide the desired product (7.3 mg, 0.010 mmol, 58% yield) as a brown solid. LC/MS (ESI) m/z: [M+H]⁺ calcd for C₃₄H₂₉F₄N₉O₂ S₂ 736.2; found 736.2; ¹H NMR (500 MHz, DMSO-d₆) δ 9.18 (s, 1H), 8.09 (s, 2H), 8.04-7.91 (m, 2H), 7.41 (dd, J=8.4, 5.3 Hz, 1H), 7.20-7.12 (m, 1H), 7.11 (d, J=37.6 Hz, 1H), 5.44-5.12 (m, 2H), 4.26-3.69 (m, 10H), 3.32-2.99 (m, 2H), 2.93-2.69 (m, 1H), 2.27-1.93 (m, 3H), 1.90-1.68 (m, 3H).

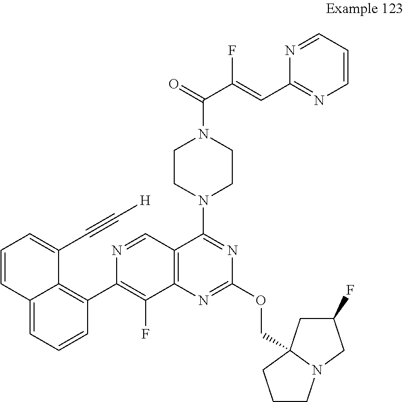 [SMILES: C#Cc1cccc2cccc(-c3ncc4c(N5CCN(C(=O)/C(F)=C/c6ncccn6)CC5)nc(OC[C@@]56CCCN5C[C@H](F)C6)nc4c3F)c12]

### (Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyrimidin-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (50 mg, 0.09 mmol), and (Z)-2-fluoro-3-(pyrimidin-2-yl)acrylic acid (31 mg, 0.19 mmol) were combined as solids and dissolved in DMF (1 mL). 1-methylimidazole (111 μL, 1.4 mmol) was added followed by chloro-N,N,N′,N′-tetramethylformamidinium hexafluorophosphate (52 mg, 0.19 mmol). The reaction mixture was stirred for 5 min and was directly purified by reverse phase HPLC (column: Xbridge C18, 19 mm×200 mm, 5 m particles; flow rate: 20 mL/min; column temperature: 25° C.; gradient: 81% 5:95 MeCN:H₂O with 10 mM AA/19% 95:5 MeCN:H₂O with 10 mM AA→100% 95:5 MeCN:H₂O with 10 mM AA; λ=220 nm) to provide the desired product (22 mg, 0.031 mmol, 34% yield) as a brown solid. LCMS (ESI) m/z: [M+H]⁺ calcd for C₃₈H₃₃F₃N₈O₂ 691.3; found 691.4; ¹H NMR (500 MHz, DMSO-d₆) δ 9.13 (s, 1H), 8.90 (d, J=4.7 Hz, 2H), 8.15 (dd, J=12.0, 8.2 Hz, 2H), 7.77-7.68 (m, 2H), 7.64-7.54 (m, 2H), 7.45 (t, J=4.9 Hz, 1H), 6.64 (d, J=35.2 Hz, 1H), 5.28 (d, J=51.3 Hz, 1H), 4.29-4.09 (m, 5H), 4.09-3.76 (m, 6H), 3.72 (s, 1H), 3.12-3.05 (m, 2H), 3.03 (br s, 1H), 2.86-2.80 (m, 1H), 2.15-1.98 (m, 3H), 1.88-1.73 (m, 3H).

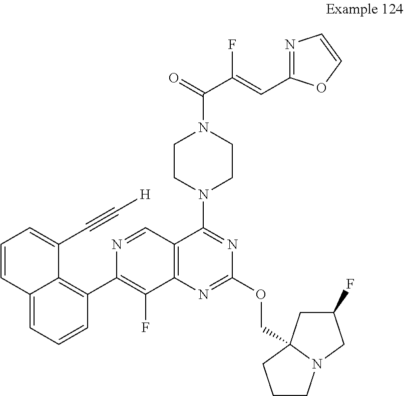 [SMILES: C#Cc1cccc2cccc(-c3ncc4c(N5CCN(C(=O)/C(F)=C/c6ncco6)CC5)nc(OC[C@@]56CCCN5C[C@H](F)C6)nc4c3F)c12]

### (Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(oxazol-2-yl)prop-2-en-1-one

7-(8-Ethynylnaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (10 mg, 0.018 mmol), and (Z)-2-fluoro-3-(oxazol-2-yl)acrylic acid (8.3 mg, 0.037 mmol) were combined as solids and dissolved in DMF (1 mL). 1-methylimidazole (22 μL, 0.277 mmol) was added followed by chloro-N,N,N′,N′-tetramethylformamidinium hexafluorophosphate (10.4 mg, 0.037 mmol). The reaction mixture was stirred for 5 min and was directly purified by reverse phase HPLC (column: Xbridge C18, 19 mm×200 mm, 5 μm particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (8.4 mg, 0.012 mmol, 67% yield) as a brown solid. LCMS (ESI) m/z: [M+H]⁺ calcd for C₃₇H₃₂F₃N₇O₃ 680.3; found 680.3; ¹H NMR (500 MHz, DMSO-d₆) δ 9.16-9.08 (m, 1H), 8.30-8.24 (m, 1H), 8.15 (dd, J=12.3, 8.1 Hz, 2H), 7.79-7.66 (m, 2H), 7.64-7.52 (m, 2H), 7.48-7.42 (m, 1H), 6.72 (d, J=35.7, 1H), 5.48-5.11 (m, 2H), 4.28-3.61 (m, 11H), 3.13-2.98 (m, 2H), 2.88-2.70 (m, 1H), 2.19-2.03 (m, 3H), 1.88-1.76 (m, 3H).

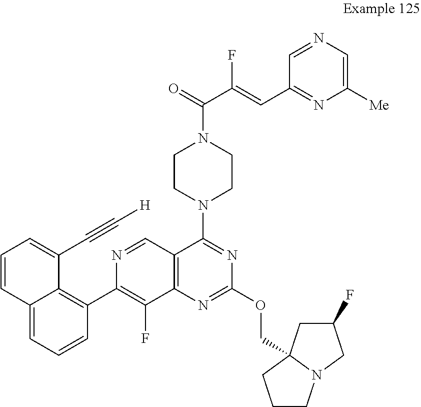 [SMILES: C#Cc1cccc2cccc(-c3ncc4c(N5CCN(C(=O)/C(F)=C/c6cncc(C)n6)CC5)nc(OC[C@@]56CCCN5C[C@H](F)C6)nc4c3F)c12]

### (Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(6-methylpyrazin-2-yl)prop-2-en-1-one

To a stirred solution of 7-(8-ethynylnaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (10 mg, 0.018 mmol), (Z)-2-fluoro-3-(6-methylpyrazin-2-yl)acrylic acid (6.7 mg, 0.037 mmol), and 1-methylimidazole (0.022 mL, 0.277 mmol) in DMF (1 mL) was added chloro-N,N,N′,N′-tetramethylformamidinium hexafluorophosphate (10.4 mg, 0.037 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 74% 5:95 MeCN:H₂O with 10 mM AA/26% 95:5 MeCN:H₂O with 10 mM AA→100% 95:5 MeCN:H₂O with 10 mM AA; λ=220 nm) to provide the desired product (9.3 mg, 0.013 mmol, 71% yield) as a brown solid. LC/MS (ESI) m/z: [M+H]⁺ calcd for C₃₉H₃₅F₃N₈O₂ 705.3 found 705.3; ¹H NMR (500 MHz, DMSO-d₆) δ 8.97 (s, 1H), 8.63 (s, 1H), 8.35 (s, 1H), 8.00 (dd, J=12.3, 8.2 Hz, 2H), 7.64-7.51 (m, 2H), 7.50-7.31 (m, 2H), 6.55 (d, J=38.5, 1H), 5.45-5.22 (m, 1H), 5.27-4.97 (m, 2H), 4.10-3.76 (m, 10H), 3.55 (s, 1H), 3.02 (s 3H), 2.96-2.78 (m, 2H), 2.73-2.60 (m, 1H), 2.05-1.80 (m, 3H), 1.73-1.51 (m, 3H).

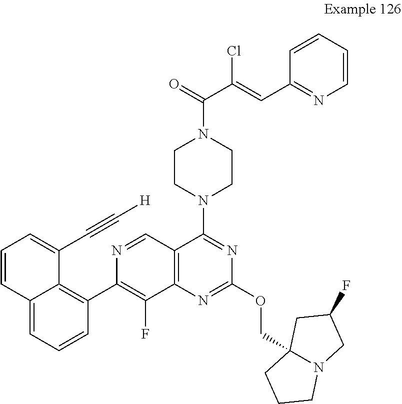 [SMILES: C#Cc1cccc2cccc(-c3ncc4c(N5CCN(C(=O)/C(Cl)=C/c6ccccn6)CC5)nc(OC[C@@]56CCCN5C[C@H](F)C6)nc4c3F)c12]

### (Z)-2-chloro-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-3-(pyridin-2-yl)prop-2-en-1-one

To a stirred solution of 7-(8-ethynylnaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (10 mg, 0.018 mmol), (Z)-2-chloro-3-(pyridin-2-yl)acrylic acid (9.7 mg, 0.037 mmol), and 1-methylimidazole (0.022 mL, 0.277 mmol) in DMF (1 mL) was added chloro-N,N,N′,N′-tetramethylformamidinium hexafluorophosphate (10.4 mg, 0.037 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 72% 5:95 MeCN:H₂O with 10 mM AA/28% 95:5 MeCN:H₂O with 10 mM AA→100% 95:5 MeCN:H₂O with 10 mM AA; λ=220 nm) to afford the desired product (13.1 mg, 0.019 mmol, 100% yield) as a brown solid. LC/MS (ESI) m/z: [M+H]⁺ calcd for C₃₉H₃₄C₁F₂N₇O₂ 706.2 found 706.1; ¹H NMR (500 MHz, DMSO-d₆) δ 9.14 (s, 1H), 8.68 (d, J=4.8 Hz, 1H), 8.15 (dd, J=12.7, 8.2 Hz, 2H), 7.96-7.89 (m, 2H), 7.77-7.67 (m, 2H), 7.63-7.53 (m, 2H), 7.46-7.37 (m, 1H), 7.23 (s, 1H), 5.54-5.08 (m, 2H), 4.36-3.72 (m, 9H), 3.72-3.64 (m, 1H), 3.46-3.06 (m, 3H), 2.99-2.80 (m, 1H), 2.39-1.96 (m, 4H), 1.87-1.68 (m, 2H).

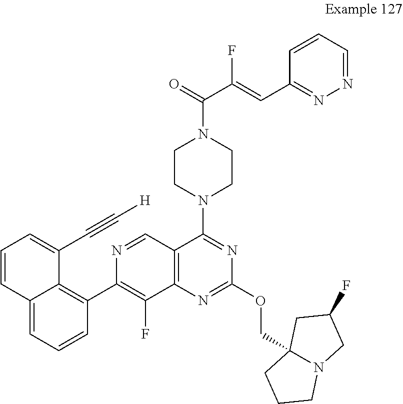 [SMILES: C#Cc1cccc2cccc(-c3ncc4c(N5CCN(C(=O)/C(F)=C/c6cccnn6)CC5)nc(OC[C@@]56CCCN5C[C@H](F)C6)nc4c3F)c12]

### (Z)-1-(4-(7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)piperazin-1-yl)-2-fluoro-3-(pyridazin-3-yl)prop-2-en-1-one

To a stirred solution of 7-(8-ethynylnaphthalen-1-yl)-8-fluoro-2-(((2R, 7aS)-2-fluorotetrahydro-1H-pyrrolizin-7a(5H)-yl)methoxy)-4-(piperazin-1-yl)pyrido[4,3-d]pyrimidine (10 mg, 0.018 mmol), (Z)-2-fluoro-3-(pyridazin-3-yl)acrylic acid (6.2 mg, 0.037 mmol), and 1-methylimidazole (0.022 mL, 0.277 mmol) in DMF (1 mL) was added chloro-N,N,N′,N′-tetramethylformamidinium hexafluorophosphate (10.4 mg, 0.037 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 95% 5:95 MeCN:H₂O with 10 mM AA/5% 95:5 MeCN:H₂O with 10 mM AA→100% 95:5 MeCN:H₂O with 10 mM AA; λ=220 nm) to provide the desired product (6.1 mg, 0.009 mmol, 48% yield) as a brown solid. LC/MS (ESI) m/z: [M+H]⁺ calcd for C₃₈H₃₃F₃N₈O₂ 691.3 found 691.1; ¹H NMR (500 MHz, CDCl₃) δ 9.15 (dd, J=5.0, 1.7 Hz, 1H), 9.09 (s, 1H), 8.05-7.93 (m, 3H), 7.77 (d, J=7.2 Hz, 1H), 7.66-7.59 (m, 2H), 7.56 (dd, J=8.6, 5.0 Hz, 1H), 7.48 (t, J=7.7 Hz, 1H), 7.13 (d, J=37.7 Hz, 1H), 5.64-5.40 (m, 1H), 5.18-4.97 (m, 1H), 4.79-4.54 (m, 1H), 4.27-3.99 (m, 10H), 3.64-3.43 (m, 1H), 3.37-3.19 (m, 1H), 2.93-2.17 (m, 7H).

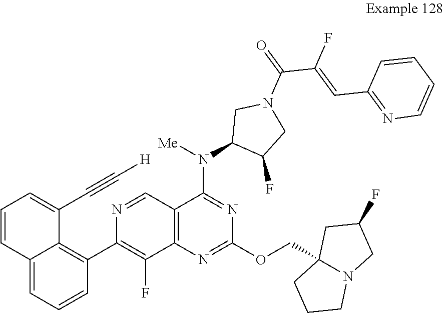 [SMILES: C#Cc1cccc2cccc(-c3ncc4c(N(C)[C@H]5CN(C(=O)/C(F)=C/c6ccccn6)C[C@H]5F)nc(OC[C@@]56CCCN5C[C@H](F)C6)nc4c3F)c12]

### (Z)-1-((3S,4R)-3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-4-fluoropyrrolidin-1-yl)-2-fluoro-3-(pyridin-2-yl)prop-2-en-1-one

To a stirred solution of 7-(8-ethynylnaphthalen-1-yl)-8-fluoro-N-((3S,4R)-4-fluoropyrrolidin-3-yl)-2-(((2R, 7aS)-2-fluorotetrahydro-1H-pyrrolizin-7a(5H)-yl)methoxy)-N-methylpyrido[4,3-d]pyrimidin-4-amine (27 mg, 0.047 mmol), (Z)-2-fluoro-3-(pyridin-2-yl)acrylic acid (15.76 mg, 0.094 mmol), and 1-methylimidazole (0.056 mL, 0.707 mmol) in acetonitrile (1 mL) was added chloro-N,N,N′,N′-tetramethylformamidinium hexafluorophosphate (26.5 mg, 0.094 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 71% 5:95 MeCN:H₂O with 10 mM AA/29% 95:5 MeCN:H₂O with 10 mM AA→100% 95:5 MeCN:H₂O with 10 mM AA; λ=220 nm) to provide the desired product (20.5 mg, 0.028 mmol, 60.2% yield) as a brown solid. LC/MS (ESI) m/z: [M+H]⁺ calcd for C₄₀H₃₅F₄N₇O₂ 722.3; found 722.3; ¹H NMR (500 MHz, DMSO-d₆) δ 9.32-9.18 (m, 1H), 8.67 (br d, J=4.1 Hz, 1H), 8.15 (dd, J=12.2, 8.2 Hz, 2H), 7.97-7.87 (m, 1H), 7.81 (br d, J=8.2 Hz, 1H), 7.76-7.66 (m, 2H), 7.64-7.54 (m, 2H), 7.40 (br t, J=6.4 Hz, 1H), 6.88 (d, J=37.5 Hz, 1H), 5.90-5.61 (m, 1H), 5.51-5.05 (m, 2H), 4.71-4.36 (m, 1H), 4.33-4.01 (m, 4H), 3.91 (s, 1H), 3.83-3.52 (m, 3H), 3.38 (br d, J=2.8 Hz, 2H), 3.10-2.75 (m, 3H), 2.30-1.42 (m, 6H).

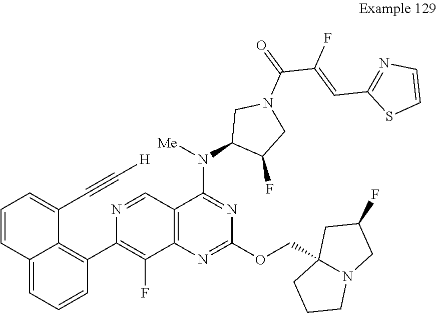 [SMILES: C#Cc1cccc2cccc(-c3ncc4c(N(C)[C@H]5CN(C(=O)/C(F)=C/c6nccs6)C[C@H]5F)nc(OC[C@@]56CCCN5C[C@H](F)C6)nc4c3F)c12]

### (Z)-1-((3S,4R)-3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-4-fluoropyrrolidin-1-yl)-2-fluoro-3-(thiazol-2-yl)prop-2-en-1-one

To a stirred solution of 7-(8-ethynylnaphthalen-1-yl)-8-fluoro-N-((3S,4R)-4-fluoropyrrolidin-3-yl)-2-(((2R, 7aS)-2-fluorotetrahydro-1H-pyrrolizin-7a(5H)-yl)methoxy)-N-methylpyrido[4,3-d]pyrimidin-4-amine (27 mg, 0.047 mmol), (Z)-2-fluoro-3-(thiazol-2-yl)acrylic acid (16.33 mg, 0.094 mmol), and 1-methylimidazole (0.056 mL, 0.707 mmol) in acetonitrile (1 mL) was added chloro-N,N,N′,N′-tetramethylformamidinium hexafluorophosphate (26.5 mg, 0.094 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 70% 5:95 MeCN:H₂O with 10 mM AA/30% 95:5 MeCN:H₂O with 10 mM AA→100% 95:5 MeCN:H₂O with 10 mM AA; λ=220 nm) to provide the desired product (21 mg, 0.029 mmol, 61% yield) as a brown solid. LC/MS (ESI) m/z: [M+H]⁺ calcd for C₃₈H₃₃F₄N₇O₂ S 728.2; found 728.3; ¹H NMR (500 MHz, DMSO-d₆) δ 9.25 (d, J=14.7 Hz, 1H), 8.15 (dd, J=12.2, 8.2 Hz, 2H), 8.03 (br d, J=7.8 Hz, 2H), 7.80-7.67 (m, 2H), 7.65-7.48 (m, 2H), 7.26 (d, J=35.5 Hz, 1H), 6.02-5.53 (m, 1H), 5.46-4.96 (m, 2H), 4.67-4.32 (m, 1H), 4.36-4.00 (m, 3H), 3.91 (s, 1H), 3.84-3.78 (m, 1H), 3.72-3.27 (m, 2H), 3.18 (d, J=5.1 Hz, 3H), 3.10-2.66 (m, 3H), 2.38-1.64 (m, 6H).

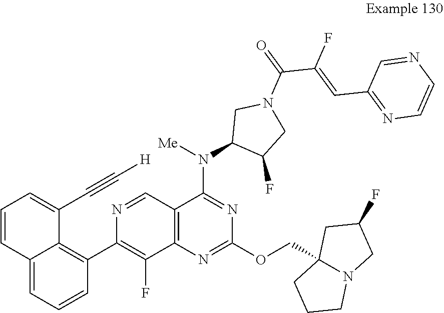 [SMILES: C#Cc1cccc2cccc(-c3ncc4c(N(C)[C@H]5CN(C(=O)/C(F)=C/c6cnccn6)C[C@H]5F)nc(OC[C@@]56CCCN5C[C@H](F)C6)nc4c3F)c12]

### (Z)-1-((3S,4R)-3-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)-4-fluoropyrrolidin-1-yl)-2-fluoro-3-(pyrazin-2-yl)prop-2-en-1-one

To a stirred solution of 7-(8-ethynylnaphthalen-1-yl)-8-fluoro-N-((3S,4R)-4-fluoropyrrolidin-3-yl)-2-(((2R, 7aS)-2-fluorotetrahydro-1H-pyrrolizin-7a(5H)-yl)methoxy)-N-methylpyrido[4,3-d]pyrimidin-4-amine (27 mg, 0.047 mmol), (Z)-2-fluoro-3-(pyrazin-2-yl)acrylic acid (15.85 mg, 0.094 mmol), and 1-methylimidazole (0.056 mL, 0.707 mmol) in acetonitrile (1 mL) was added chloro-N,N,N′,N′-tetramethylformamidinium hexafluorophosphate (26.5 mg, 0.094 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 77% 5:95 MeCN:H₂O with 10 mM AA/23% 95:5 MeCN:H₂O with 10 mM AA→100% 95:5 MeCN:H₂O with 10 mM AA; λ=220 nm) to provide the desired product (22 mg, 0.030 mmol, 634% yield) as a brown solid. LC/MS (ESI) m/z: [M+H]⁺ calcd for C₃₉H₃₄F₄N₈O₂ 723.3; found 723.3; ¹HNMR (500 MHz, DMSO-d₆) δ 9.31-9.18 (m, 1H), 8.99 (s, 1H), 8.75 (s, 1H), 8.62 (s, 1H), 8.15 (dd, J=12.2, 8.2 Hz, 2H), 7.80-7.67 (m, 2H), 7.65-7.44 (m, 2H), 6.97 (d, J=37.3, 1H), 5.97-5.58 (m, 1H), 5.51-4.92 (m, 2H), 4.67-4.37 (m, 1H), 4.32-3.94 (m, 2H), 3.82 (s, 1H), 3.76-3.42 (m, 4H), 3.35 (s, 3H), 2.55 (s, 3H), 2.33-1.62 (m, 6H).

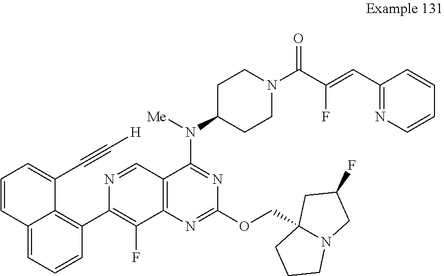

### (Z)-1-(4-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)piperidin-1-yl)-2-fluoro-3-(pyridin-2-yl)prop-2-en-1-one

To a stirred solution of 7-(8-ethynylnaphthalen-1-yl)-8-fluoro-2-(((2R, 7aS)-2-fluorotetrahydro-1H-pyrrolizin-7a(5H)-yl)methoxy)-N-methyl-N-(piperidin-4-yl)pyrido[4,3-d]pyrimidin-4-amine (26 mg, 0.046 mmol), (Z)-2-fluoro-3-(pyridin-2-yl)acrylic acid (15.3 mg, 0.091 mmol), and 1-methylimidazole (0.055 mL, 0.686 mmol) in acetonitrile (1 mL) was added chloro-N,N,N′,N′-tetramethylformamidinium hexafluorophosphate (25.7 mg, 0.091 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 75% 5:95 MeCN:H₂O with 10 mM AA/25% 95:5 MeCN:H₂O with 10 mM AA→100% 95:5 MeCN:H₂O with 10 mM AA; λ=220 nm) to provide the desired product (19.8 mg, 0.028 mmol, 60.3% yield) as a brown solid. LC/MS (ESI) m/z: [M+H]⁺ calcd for C₄₁H₃₈F₃N₇O₂ 718.3; found 718.3; 1HNMR (500 MHz, DMSO-d₆) δ 9.17 (s, 1H), 8.65 (d, J=4.8 Hz, 1H), 8.23-8.07 (m, 2H), 7.89 (td, J=7.8, 1.8 Hz, 1H), 7.79 (d, J=8.0 Hz, 1H), 7.71 (dd, J=16.2, 7.7 Hz, 2H), 7.62-7.51 (m, 2H), 7.37 (ddd, J=7.5, 4.8, 1.1 Hz, 1H), 6.59 (d, J=39.2, 1H), 5.53-5.15 (m, 1H), 5.08-4.78 (m, 1H), 4.63-4.35 (m, 1H), 4.33-3.99 (m, 3H), 3.76 (s, 1H), 3.48-3.32 (m, 3H), 3.17 (d, J=5.2 Hz, 3H), 3.08-2.57 (m, 3H), 2.33-1.55 (m, 10H).

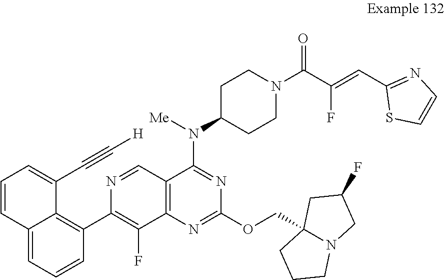

### (Z)-1-(4-((7-(8-ethynylnaphthalen-1-yl)-8-fluoro-2-(((2R, 7aS)-2-fluorotetrahydro-1H-pyrrolizin-7a(5H)-yl)methoxy)pyrido[4,3-d]pyrimidin-4-yl)(methyl)amino)piperidin-1-yl)-2-fluoro-3-(thiazol-2-yl)prop-2-en-1-one

To a stirred solution of 7-(8-ethynylnaphthalen-1-yl)-8-fluoro-2-(((2R, 7aS)-2-fluorotetrahydro-1H-pyrrolizin-7a(5H)-yl)methoxy)-N-methyl-N-(piperidin-4-yl)pyrido[4,3-d]pyrimidin-4-amine (26 mg, 0.046 mmol), (Z)-2-fluoro-3-(thiazol-2-yl)acrylic acid (15.8 mg, 0.091 mmol), and 1-methylimidazole (0.055 mL, 0.686 mmol) in acetonitrile (1 mL) was added chloro-N,N,N′,N′-tetramethylformamidinium hexafluorophosphate (25.7 mg, 0.091 mmol). The mixture was stirred for 15 min and was purified by reverse phase HPLC (column: Xbridge C18, 19 mm×200 mm, 5 m particles; flow rate: 20 mL/min; column temperature: 25° C.; gradient: 72% 5:95 MeCN:H₂O with 10 mM AA/28% 95:5 MeCN:H₂O with 10 mM AA→100% 95:5 MeCN:H₂O with 10 mM AA; λ=220 nm) to provide the desired product (17.9 mg, 0.025 mmol, 54.1% yield). LC/MS (ESI) m/z: [M+H]⁺ calcd for C₃₉H₃₆F₃N₇O₂ S 724.3; found 724.3; 1HNMR (500 MHz, DMSO-d₆) δ 9.18 (s, 1H), 8.22-8.06 (m, 2H), 8.03-7.94 (m, 2H), 7.79-7.67 (m, 2H), 7.64-7.52 (m, 2H), 7.06 (d, J=38.3 Hz 1H), 5.49-5.14 (m, 1H), 4.87 (s, 1H), 4.60-4.30 (m, 1H), 4.29-3.96 (m, 3H), 3.76 (br d, J=1.7 Hz, 1H), 3.64-3.28 (m, 3H), 3.18 (d, J=5.1 Hz, 3H), 2.99-2.54 (m, 3H), 2.40-1.44 (m, 10H).

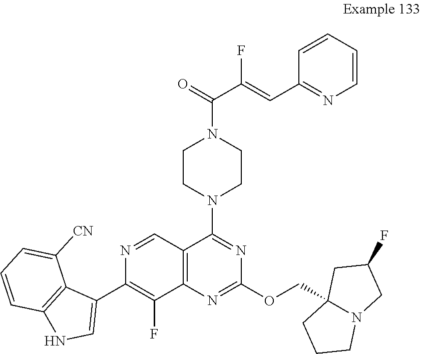 [SMILES: N#Cc1cccc2[nH]cc(-c3ncc4c(N5CCN(C(=O)/C(F)=C/c6ccccn6)CC5)nc(OC[C@@]56CCCN5C[C@H](F)C6)nc4c3F)c12]

### 3-(8-fluoro-4-(4-((Z)-2-fluoro-3-(pyridin-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7-yl)-1H-indole-4-carbonitrile

3-(8-Fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)-1H-indole-4-carbonitrile (20 mg, 0.033 mmol) and (Z)-2-fluoro-3-(pyridin-2-yl)acrylic acid (17 mg, 0.1 mmol) were combined as solids and dissolved in DMF (0.5 mL). 1-methylimidazole (14 μL, 0.2 mmol) was added followed by chloro-N,N,N′,N′-tetramethylformamidinium hexafluorophosphate (28 mg, 0.1 mmol). The reaction mixture was stirred for 5 min. The solution was directly purified by reverse phase HPLC (column: Xbridge C18, 19 mm×200 mm, 5 m particles; flow rate: 20 mL/min; column temperature: 25° C.; gradient: 100% 5:95 MeCN:H₂O with 0.1% TFA/0% 95:5 MeCN:H₂O with 0.1% TFA→100% 95:5 MeCN:H₂O with 0.1% TFA; λ=220 nm) to provide the desired product, a brown solid, as the bis TFA salt (13.2 mg, 0.014 mmol, 44% yield). LCMS (ESI) m/z: [M+H]⁺ calcd for C₃₆H₃₃F₃N₉O₂ 680.3; found 680.2; 1HNMR (500 MHz, DMSO-d₆) δ 12.36 (br s, 1H), 9.23 (s, 1H), 8.65 (br d, J=5.0 Hz, 1H), 8.09 (d, J=2.5 Hz, 1H), 7.89 (br d, J=8.1 Hz, 2H), 7.79 (d, J=7.9 Hz, 1H), 7.63 (d, J=7.1 Hz, 1H), 7.42-7.34 (m, 2H), 6.67 (d, J=38.6 Hz, 1H), 5.58 (d, J=52.3 Hz, 1H), 4.68-4.58 (m, 2H), 4.24-4.10 (m, 4H), 4.03-3.93 (m, 2H), 3.88-3.71 (m, 5H), 3.24-3.17 (m, 2H), 2.39-1.97 (m, 5H).

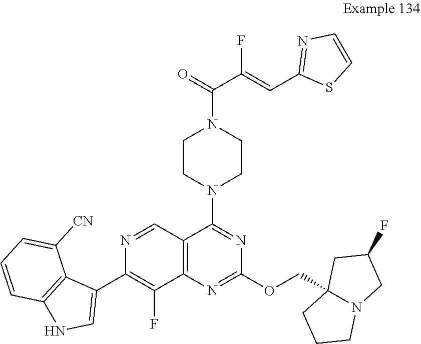 [SMILES: N#Cc1cccc2[nH]cc(-c3ncc4c(N5CCN(C(=O)/C(F)=C/c6nccs6)CC5)nc(OC[C@@]56CCCN5C[C@H](F)C6)nc4c3F)c12]

### 3-(8-fluoro-4-(4-((Z)-2-fluoro-3-(thiazol-2-yl)acryloyl)piperazin-1-yl)-2-(((2R, 7aS)-2-fluorotetrahydro-1H-pyrrolizin-7a(5H)-yl)methoxy)pyrido[4,3-d]pyrimidin-7-yl)-1H-indole-4-carbonitrile

3-(8-Fluoro-2-(((2R, 7aS)-2-fluorotetrahydro-1H-pyrrolizin-7a(5H)-yl)methoxy)-4-(piperazin-1-yl)pyrido[4,3-d]pyrimidin-7-yl)-1H-indole-4-carbonitrile (20 mg, 0.033 mmol) and (Z)-2-fluoro-3-(pyridin-2-yl)acrylic acid (17 mg, 0.1 mmol)were combined as solids and dissolved in DMF (0.5 mL). 1-methylimidazole (14 μL, 0.2 mmol) was added followed by chloro-N,N,N′,N′-tetramethylformamidinium hexafluorophosphate (28 mg, 0.1 mmol). The reaction mixture was stirred for 5 min. The solution was directly purified by reverse phase HPLC (column: Xbridge C18, 19 mm×200 mm, 5 m particles; flow rate: 20 mL/min; column temperature: 25° C.; gradient: 87% 5:95 MeCN:H₂O with 10 mM AA/13% 95:5 MeCN:H₂O with 10 mM AA→100% 95:5 MeCN:H₂O with 10 mM AA; λ=220 nm) to provide the desired product (4.1 mg, 0.006 mmol, 21% yield) as a brown solid. LCMS (ESI) m/z: [M+H]⁺ calcd for C₃₄H₃₁F₃N₉O₂ S 686.3; found 686.2; ¹H NMR (500 MHz, DMSO-d₆) δ 12.29 (br s, 1H), 9.16 (s, 1H), 8.08 (d, J=2.3 Hz, 1H), 8.03-7.97 (m, 2H), 7.88 (d, J=8.2 Hz, 1H), 7.62 (d, J=7.2 Hz, 1H), 7.35 (t, J=7.8 Hz, 1H), 7.10 (d, J=37.5 Hz, 1H), 5.31 (d, J=54.9 Hz, 1H), 4.22-4.05 (m, 6H), 4.03-3.80 (m, 4H), 3.18-3.02 (m, 3H), 2.90-2.80 (m, 1H), 1.96 (s, 6H).

### Biological Activity

### KRAS^{G12C} RAF Disruption Assay

This is a functional assay that measures activity of compounds against KRAS-G12C^{(ON)}, i.e., the active form of KRAS G12C. Recombinant GMPPNP-loaded KRAS G12C (5 nM) was treated with compound at room temperature for 20 minutes in assay buffer (50 mM Tris pH 7.5, 100 mM NaCl, 1 mM MgCl₂, 1 mM DTT, 100ug/ml BSA). Recombinant GST-RAF1 RBD (9 nM) was added, and the reaction mixture was incubated for 20 minutes. SA-Tb (0.25 nM) was added, and the reaction mixture was incubated for 3 hours. HTRF signal was measured (PerkinElmer Envision), the signal ratio (λₑₘ 520/λₑₘ 495) was calculated, and IC₅₀ values were calculated from the dose-response curve.

The IC₅₀ values for compounds described herein are shown in Table 1.

TABLE 1

| | KRASG12C RAF | |
|---|---|---|
| | Disruption Assay | |
| Example No. | (IC₅₀) | |
| 1 | 27 | nM |
| 2 | 32 | nM |
| 3 | 19 | nM |
| 4 | 260 | nM |
| 5 | 79 | nM |
| 6 | 45 | nM |
| 7 | 16 | nM |
| 8 | 1300 | nM |
| 9 | 350 | nM |
| 10 | 24 | nM |
| 11 | 450 | nM |
| 12 | 11 | nM |
| 13 | 38 | nM |
| 14 | 22 | nM |
| 15 | 60 | nM |
| 16 | 180 | nM |
| 17 | 2600 | nM |
| 18 | 25 | nM |
| 19A | 59 | nM |
| 19B | 270 | nM |
| 20 | 89 | nM |
| 21A | 67 | nM |
| 21B | 330 | nM |
| 22 | 1900 | nM |
| 23 | 180 | nM |
| 24 | 110 | nM |
| 25 | 290 | nM |
| 26 | 170 | nM |
| 27 | 3 | nM |
| 28 | 4 | nM |
| 29 | 100 | nM |
| 30 | 470 | nM |
| 31 | 64 | nM |
| 32 | 76 | nM |
| 33 | 75 | nM |
| 34 | 400 | nM |
| 35 | 430 | nM |
| 36 | 150 | nM |
| 37 | 130 | nM |
| 38 | 4700 | nM |
| 39 | 5100 | nM |
| 40 | 880 | nM |
| 41 | 760 | nM |
| 42 | 760 | nM |
| 43 | 1100 | nM |
| 44 | 360 | nM |
| 45 | 460 | nM |
| 46 | 390 | nM |
| 47 | 100 | nM |
| 48 | 780 | nM |
| 49 | 800 | nM |
| 50 | 840 | nM |
| 51 | 360 | nM |
| 52 | 430 | nM |
| 53 | 44 | nM |
| 54 | 5 | nM |
| 55 | 2 | nM |
| 56 | 6 | nM |
| 57 | 4 | nM |
| 58 | 2 | nM |
| 59 | 2 | nM |
| 60 | 180 | nM |
| 61 | 300 | nM |
| 62 | 300 | nM |
| 63 | 180 | nM |
| 64 | 7 | nM |
| 65 | 4.5 | nM |
| 66 | <2 | nM |
| 67 | 41 | nM |
| 68 | 220 | nM |
| 69 | 300 | nM |
| 70 | 26 | nM |
| 71 | 40 | nM |
| 72 | 440 | nM |
| 73 trans isomer 1 | 1600 | nM |
| 73 trans isomer 2 | 5700 | nM |
| 73 cis isomer 1 | 110 | nM |
| 73 cis isomer 2 | 1800 | nM |
| 74 trans isomer 1 | 2.5 | nM |
| 74 trans isomer 2 | 110 | nM |
| 74 cis isomer 1 | 1000 | nM |
| 74 cis isomer 2 | 910 | nM |
| 75 | 57 | nM |
| 76 | 590 | nM |
| 77 | 120 | nM |
| 78 | 120 | nM |
| 79 | 65 | nM |
| 80 | 30 | nM |
| 81 | 57 | nM |
| 82 | 62 | nM |
| 83 | 2600 | nM |
| 84 | 3500 | nM |
| 85 | 110 | nM |
| 86 | 110 | nM |
| 87 | 1600 | nM |
| 88 | 1000 | nM |
| 89 | 37 | nM |
| 90 | 110 | nM |
| 91 | 5000 | nM |
| 92 | 24000 | nM |
| 93 | 3300 | nM |
| 94 | 34 | nM |
| 95 | 14 | nM |
| 96 | 110 | nM |
| 97 | 550 | nM |
| 98 | 91 | nM |
| 99 | 70 | nM |
| 100 | 5300 | nM |
| 101 | 12000 | nM |
| 102 | 87 | nM |
| 103 | 620 | nM |
| 104 | 16000 | nM |
| 105 | 9700 | nM |
| 106 | 1600 | nM |
| 107 | 5600 | nM |
| 108 | 4300 | nM |
| 109 | 240 | nM |
| 110 | 180 | nM |
| 111 | 11 | nM |
| 112 | <2 | nM |
| 113 | 156 | nM |
| 114 | 719 | nM |
| 115 | 14 | nM |
| 116 | 27 | nM |
| 117 | 9 | nM |
| 118 | 30 | nM |
| 119 | 5 | nM |
| 120 | 30 | nM |
| 121 | 8 | nM |
| 122 | 6 | nM |
| 123 | 187 | nM |
| 124 | 540 | nM |
| 125 | 480 | nM |
| 126 | 47000 | nM |
| 127 | 10 | nM |
| 128 | 30 | nM |
| 129 | 200 | nM |
| 130 | 60 | nM |
| 131 | 4200 | nM |
| 132 | 16800 | nM |
| 133 | 890 | nM |
| 134 | 970 | nM |

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

All of the references cited herein are incorporated herein by reference in their entireties.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1. A compound of formula (I): 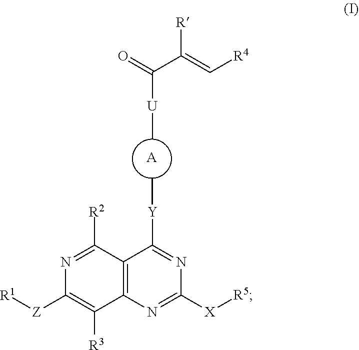
or a pharmaceutically acceptable salt thereof, wherein:
U is a bond or NH;
Z is a bond, O, NR^{e} or CR^{e}R^{f}, wherein R^{e} and R^{f} are independently hydrogen or C₁-C₃alkyl;
R¹ is aryl or heteroaryl, wherein the aryl and the heteroaryl are optionally substituted with one, two, three, four, or five substituents independently selected from C₁-C₃alkoxy, C₁-C₃alkyl, C₂-C₄alkenyl, C₂-C₄alkynyl, amino, amino C₁-C₃alkyl, cyano, C₃-C₄cycloalkyl, halo, halo C₁-C₃alkyl, hydroxy, and hydroxy C₁-C₃alkyl;
R² and R³ are independently selected from hydrogen, C₁-C₃alkoxy, C₁-C₃alkyl, cyano, halo, halo C₁-C₃alkyl, —C(O)NH₂, —C(O)NH(C₁-C₃alkyl), —C(O)N(C₁-C₃alkyl)₂, and hydroxy;
Y is a bond, O, NR^{g}(CR^{e}R^{f})m, NR^{f}, or CR^{e}R^{f}, wherein m is 1, 2, or 3, and wherein R^{e}, R^{f}, and R^{g} are independently hydrogen or C₁-C₃alkyl;
A is a four- to ten-membered nitrogen-containing monocyclic or bicyclic bridged, fused, or spirocyclic saturated, unsaturated, or partially unsaturated ring system optionally containing one or two heteroatoms independently selected from nitrogen, oxygen, and sulfur, wherein the ring system is optionally substituted with one, two, or three groups independently selected from C₁-C₃alkoxy, C₁-C₃alkoxyalkyl, C₁-C₃alkyl, cyano, cyano C₁-C₃alkyl, halo, halo C₁-C₃alkyl, amino, amino C₁-C₃alkyl, hydroxy, hydroxy C₁-C₃alkyl, and oxo;
R′ is halo;
R⁴ is an aryl or heteroaryl ring; wherein the ring is optionally substituted with one, two, or three substituents independently selected from C₂-C₄alkenyl, C₁-C₃alkoxy, C₁-C₃alkoxy C₁-C₆alkyl, C₁-C₃alkyl, cyano, cyano C₁-C₃alkyl, halo, haloC₁-C₃alkoxy, halo C₁-C₃alkyl, nitro, and oxo;
X is O or NR¹⁶, wherein R¹⁶ is hydrogen or C₁-C₃alkyl;
R⁵ is selected from hydrogen, C₁-C₆alkoxy C₁-C₆alkyl, C₁-C₆alkyl, aryl, aryl C₁-C₆alkyl, carboxy C₁-C₆alkyl, C₃-C₆cycloalkyl, C₃-C₆cycloalkyl C₁-C₆alkyl, di(C₁-C₃alkyl)amino C₂-C₆alkyl, halo C₁-C₆alkyl, heteroaryl, heteroaryl C₁-C₆alkyl, heterocyclyl, heterocyclyl C₁-C₆alkyl, hydroxy C₁-C₆alkyl, NR^{a}R^{b}—C(O)—C₁-C₆alkyl), NR^{a}R^{b}C₁-C₆alkyl, wherein the aryl, the aryl part of the aryl C₁-C₆alkyl, the C₃-C₆cycloalkyl, the cycloalkyl part of the C₃-C₆cycloalkyl C₁-C₆alkyl, the heteroaryl, the heteroaryl part of the heteroaryl C₁-C₆alkyl, the heterocyclyl, the heterocyclyl part of the heterocyclyl C₁-C₆alkyl, are optionally substituted with one, two, three, or four groups independently selected from C₁-C₃alkoxy, C₁-C₃alkyl, (C₁-C₆alkyl)amino, (C₁-C₆alkyl)amino C₁-C₃alkyl, amino, amino C₁-C₃alkyl, azido C₁-C₆alkyl, carboxy, cyano, di(C₁-C₆alkyl)amino, di(C₁-C₆alkyl)amino C₁-C₃alkyl, halo, halo C₁-C₃alkoxy, halo C₁-C₃alkyl, heteroaryl C₁-C₃alkyl, heterocyclyl, heterocyclyl C₁-C₃alkyl, hydroxy, hydroxy C₁-C₃alkyl, nitro, and oxo; wherein the heteroaryl part of the heteroaryl C₁-C₃alkyl, the heterocyclyl, and the heterocyclyl part of the heterocyclyl C₁-C₃alkyl is further optionally substituted with one, two, or three groups independently selected from C₁-C₃alkoxy, C₁-C₃alkyl, halo, halo C₁-C₃alkyl, and (NR^{X}R^{y})C₁-C₃alkyl; or
R⁵ and R¹⁶, together with the nitrogen atom to which they are attached, form a heterocyclic group optionally substituted with one, two, three, four, or five groups independently selected from one, two, three, or four groups independently selected from C₁-C₃alkoxy, C₁-C₃alkoxyalkyl, C₁-C₃alkyl, amino, amino C₁-C₃alkyl, hydroxy, and hydroxy C₁-C₃alkyl;
one of R^{a} and R^{b} is selected from hydrogen and C₁-C₃alkyl and the other is selected from hydrogen, C₁-C₃alkyl, C₁-C₃alkoxycarbonyl, C₁-C₃alkylcarbonyl, aryl C₁-C₆alkyl, C₃-C₆cycloalkyl, and C₃-C₆cycloalkyl C₁-C₆alkyl; and
one of R^{X} and R^{y} is hydrogen and the other is selected from —C(O)C₁-C₆alkylheterocyclyl, wherein the heterocycyl is optionally substituted with an oxo group.

2. A compound of claim 1, or a pharmaceutically acceptable salt thereof, wherein:
U is a bond or NH;
Z is a bond, O, NR^{e} or CR^{e}R^{f}, wherein R^{e} and R^{f} are independently hydrogen or C₁-C₃alkyl;
R¹ is aryl or heteroaryl, wherein the aryl and the heteroaryl are optionally substituted with one, two, three, four, or five substituents independently selected from C₁-C₃alkoxy, C₁-C₃alkyl, C₂-C₄alkenyl, C₂-C₄alkynyl, amino, amino C₁-C₃alkyl, cyano, C₃-C₄cycloalkyl, halo, halo C₁-C₃alkyl, hydroxy, and hydroxy C₁-C₃alkyl;
R² and R³ are independently selected from hydrogen, C₁-C₃alkoxy, C₁-C₃alkyl, cyano, halo, halo C₁-C₃alkyl, —C(O)NH₂, —C(O)NH(C₁-C₃alkyl), —C(O)N(C₁-C₃alkyl)₂, and hydroxy;
Y is a bond, O, NR^{g}(CR^{e}R^{f})m, NR^{f}, or CR^{e}R^{f}, wherein m is 1, 2, or 3, and wherein R^{e}, R^{f}, and R^{g} are independently hydrogen or C₁-C₃alkyl;
A is a four- to ten-membered nitrogen-containing monocyclic or bicyclic bridged, fused, or spirocyclic saturated, unsaturated, or partially unsaturated ring system optionally containing one or two heteroatoms independently selected from nitrogen, oxygen, and sulfur, wherein the ring system is optionally substituted with one, two, or three groups independently selected from C₁-C₃alkoxy, C₁-C₃alkoxyalkyl, C₁-C₃alkyl, cyano, cyano C₁-C₃alkyl, halo, halo C₁-C₃alkyl, amino, amino C₁-C₃alkyl, hydroxy, hydroxy C₁-C₃alkyl, and oxo;
R′ is halo;
R⁴ is an aryl or heteroaryl ring; wherein the ring is optionally substituted with one, two, or three substituents independently selected from C₂-C₄alkenyl, C₁-C₃alkoxy, C₁-C₃alkoxy C₁-C₆alkyl, C₁-C₃alkyl, cyano, cyano C₁-C₃alkyl, halo, haloC₁-C₃alkoxy, halo C₁-C₃alkyl, nitro, and oxo;
X is O or NR¹⁶, wherein R¹⁶ is hydrogen or C₁-C₃alkyl;
R⁵ is selected from hydrogen, C₁-C₆alkoxy C₁-C₆alkyl, C₁-C₆alkyl, aryl, aryl C₁-C₆alkyl, carboxy C₁-C₆alkyl, C₃-C₆cycloalkyl, C₃-C₆cycloalkyl C₁-C₆alkyl, di(C₁-C₃alkyl)amino C₂-C₆alkyl, halo C₁-C₆alkyl, heteroaryl, heteroaryl C₁-C₆alkyl, heterocyclyl, heterocyclyl C₁-C₆alkyl, hydroxy C₁-C₆alkyl, NR^{a}R^{b}—C(O)—C₁-C₆alkyl), NR^{a}R^{b}C₁-C₆alkyl, wherein the aryl, the aryl part of the aryl C₁-C₆alkyl, the C₃-C₆cycloalkyl, the cycloalkyl part of the C₃-C₆cycloalkyl C₁-C₆alkyl, the heteroaryl, the heteroaryl part of the heteroaryl C₁-C₆alkyl, the heterocyclyl, the heterocyclyl part of the heterocyclyl C₁-C₆alkyl, are optionally substituted with one, two, three, or four groups independently selected from C₁-C₃alkoxy, C₁-C₃alkyl, (C₁-C₆alkyl)amino, (C₁-C₆alkyl)amino C₁-C₃alkyl, amino, amino C₁-C₃alkyl, carboxy, cyano, di(C₁-C₆alkyl)amino, di(C₁-C₆alkyl)amino C₁-C₃alkyl, halo, halo C₁-C₃alkoxy, halo C₁-C₃alkyl, heterocyclyl, heterocyclyl C₁-C₃alkyl, hydroxy, hydroxy C₁-C₃alkyl, nitro, and oxo; wherein the heterocyclyl, and the heterocyclyl part of the heterocyclyl C₁-C₃alkyl is further optionally substituted with one, two, or three groups independently selected from C₁-C₃alkoxy, C₁-C₃alkyl, halo, and halo C₁-C₃alkyl; or
R⁵ and R¹⁶, together with the nitrogen atom to which they are attached, form a heterocyclic group optionally substituted with one, two, three, four, or five groups independently selected from one, two, three, or four groups independently selected from C₁-C₃alkoxy, C₁-C₃alkoxyalkyl, C₁-C₃alkyl, amino, amino C₁-C₃alkyl, hydroxy, and hydroxy C₁-C₃alkyl; and
one of R^{a} and R^{b} is selected from hydrogen and C₁-C₃alkyl and the other is selected from hydrogen, C₁-C₃alkyl, C₁-C₃alkoxycarbonyl, C₁-C₃alkylcarbonyl, aryl C₁-C₆alkyl, C₃-C₆cycloalkyl, and C₃-C₆cycloalkyl C₁-C₆alkyl.

3. A compound of claim 1, or a pharmaceutically acceptable salt thereof, wherein:
U is a bond or NH;
Z is a bond, O, NR^{e} or CR^{e}R^{f}, wherein R^{e} and R^{f} are independently hydrogen or C₁-C₃alkyl;
R¹ is aryl or heteroaryl, wherein the aryl and the heteroaryl are optionally substituted with one, two, three, four, or five substituents independently selected from C₁-C₃alkoxy, C₁-C₃alkyl, C₂-C₄alkenyl, C₂-C₄alkynyl, amino, amino C₁-C₃alkyl, cyano, C₃-C₄cycloalkyl, halo, halo C₁-C₃alkyl, hydroxy, and hydroxy C₁-C₃alkyl;
R² and R³ are independently selected from hydrogen, C₁-C₃alkoxy, C₁-C₃alkyl, cyano, halo, halo C₁-C₃alkyl, —C(O)NH₂, —C(O)NH(C₁-C₃alkyl), —C(O)N(C₁-C₃alkyl)₂, and hydroxy;
Y is a bond, O, NR^{g}(CR^{e}R^{f})m, NR^{f}, or CR^{e}R^{f}, wherein m is 1, 2, or 3, and wherein R^{e}, R^{f}, and R^{g} are independently hydrogen or C₁-C₃alkyl;
A is a four- to ten-membered nitrogen-containing monocyclic or bicyclic bridged, fused, or spirocyclic saturated, unsaturated, or partially unsaturated ring system optionally containing one or two heteroatoms independently selected from nitrogen, oxygen, and sulfur, wherein the ring system is optionally substituted with one, two, or three groups independently selected from C₁-C₃alkoxy, C₁-C₃alkoxyalkyl, C₁-C₃alkyl, cyano, halo, halo C₁-C₃alkyl, amino, amino C₁-C₃alkyl, hydroxy, hydroxy C₁-C₃alkyl, and oxo;
R′ is halo;
R⁴ is a five- or six-membered aromatic ring optionally containing one, two, or three heteroatoms independently selected from nitrogen, oxygen, and sulfur; wherein the ring is optionally substituted with one, two, or three substituents independently selected from C₂-C₄alkenyl, C₁-C₃alkyl, cyano, cyano C₁-C₃alkyl, halo, halo C₁-C₃alkoxy, halo C₁-C₃alkyl, nitro, and oxo;
X is O or NR¹⁶, wherein R¹⁶ is hydrogen or C₁-C₃alkyl;
R⁵ is selected from hydrogen, C₁-C₆alkoxy C₁-C₆alkyl, C₁-C₆alkyl, aryl, aryl C₁-C₆alkyl, carboxy C₁-C₆alkyl, C₃-C₆cycloalkyl, C₃-C₆cycloalkyl C₁-C₆alkyl, di(C₁-C₃alkyl)amino C₂-C₆alkyl, halo C₁-C₆alkyl, heteroaryl, heteroaryl C₁-C₆alkyl, heterocyclyl, heterocyclyl C₁-C₆alkyl, hydroxy C₁-C₆alkyl, NR^{a}R^{b}—C(O)—C₁-C₆alkyl), NR^{a}R^{b}C₁-C₆alkyl, wherein the aryl, the aryl part of the aryl C₁-C₆alkyl, the C₃-C₆cycloalkyl, the cycloalkyl part of the C₃-C₆cycloalkyl C₁-C₆alkyl, the heteroaryl, the heteroaryl part of the heteroaryl C₁-C₆alkyl, the heterocyclyl, the heterocyclyl part of the heterocyclyl C₁-C₆alkyl, are optionally substituted with one, two, three, or four groups independently selected from C₁-C₃alkoxy, C₁-C₃alkyl, (C₁-C₆alkyl)amino, (C₁-C₆alkyl)amino C₁-C₃alkyl, amino, amino C₁-C₃alkyl, carboxy, cyano, di(C₁-C₆alkyl)amino, di(C₁-C₆alkyl)amino C₁-C₃alkyl, halo, halo C₁-C₃alkoxy, halo C₁-C₃alkyl, heterocyclyl, heterocyclyl C₁-C₃alkyl, hydroxy, hydroxy C₁-C₃alkyl, nitro, and oxo; wherein the heterocyclyl and the heterocyclyl part of the heterocyclyl C₁-C₃alkyl is further optionally substituted with one, two, or three groups independently selected from C₁-C₃alkoxy, C₁-C₃alkyl, halo, and halo C₁-C₃alkyl; or
R⁵ and R¹⁶, together with the nitrogen atom to which they are attached, form a heterocyclic group optionally substituted with one, two, three, four, or five groups independently selected from one, two, three, or four groups independently selected from C₁-C₃alkoxy, C₁-C₃alkoxyalkyl, C₁-C₃alkyl, amino, amino C₁-C₃alkyl, hydroxy, and hydroxy C₁-C₃alkyl; and
one of R^{a} and R^{b} is selected from hydrogen and C₁-C₃alkyl and the other is selected from hydrogen, C₁-C₃alkyl, C₁-C₃alkoxycarbonyl, C₁-C₃alkylcarbonyl, aryl C₁-C₆alkyl, C₃-C₆cycloalkyl, and C₃-C₆cycloalkyl C₁-C₆alkyl.

4. The compound of claim 1, or a pharmaceutically acceptable salt thereof, wherein R⁴ is a five- or six-membered aromatic ring optionally containing one, two, or three heteroatoms independently selected from nitrogen, oxygen, and sulfur.

5. The compound of claim 1, or a pharmaceutically acceptable salt thereof, wherein Y is a bond.

6. The compound of claim 1, or a pharmaceutically acceptable salt thereof, wherein Y is NR^{f}.

7. The compound of claim 1, or a pharmaceutically acceptable salt thereof, wherein Y is NCH₃.

8. The compound of claim 1, or a pharmaceutically acceptable salt thereof, wherein A is a four- to nine-membered monocyclic or bicyclic bridged, spirocyclic, or fused saturated ring system optionally containing one or two nitrogen atoms.

9. The compound of claim 1, or a pharmaceutically acceptable salt thereof, wherein A-U is 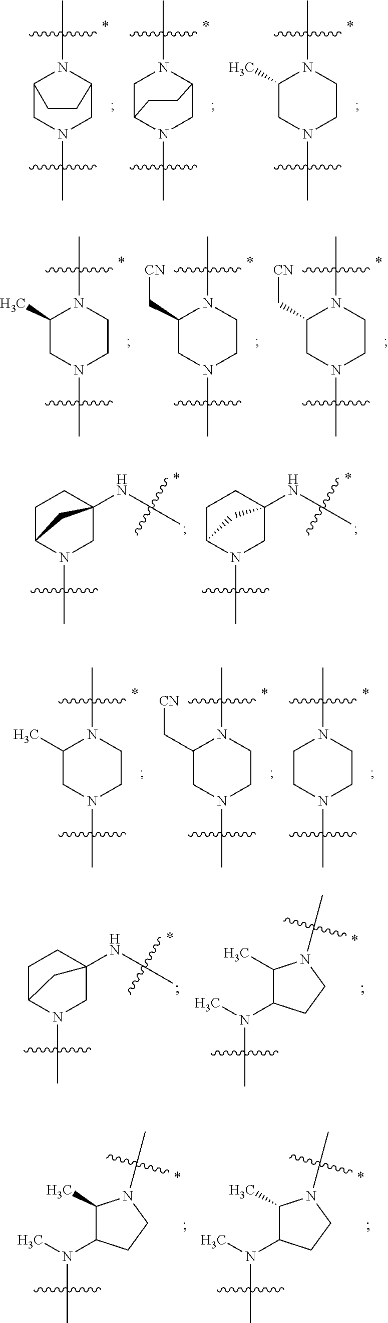 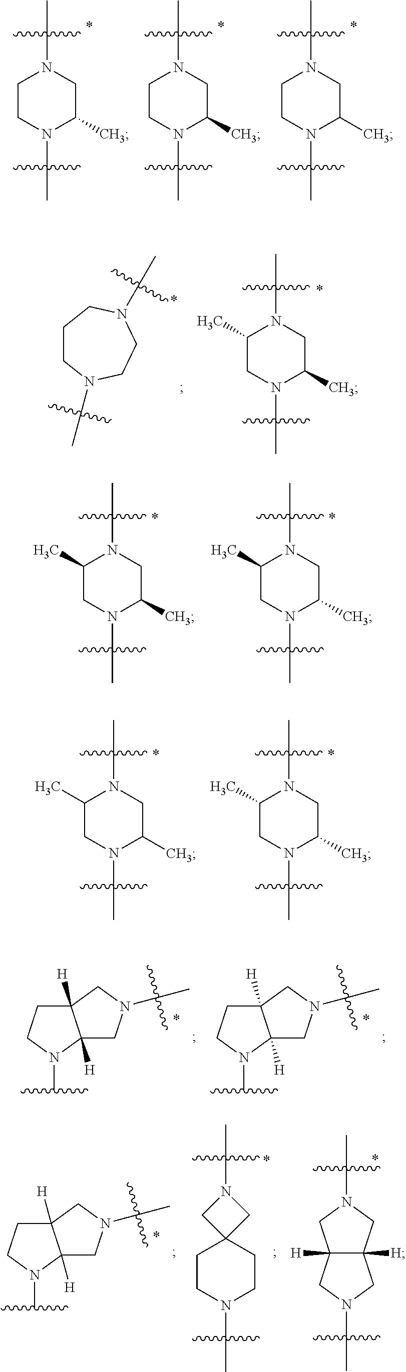 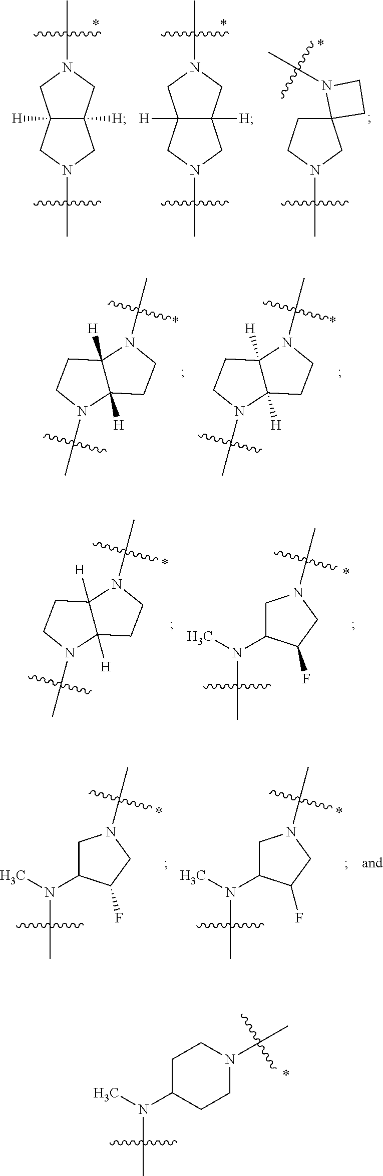
wherein [symbol]
* represents the point of attachment to the carbonyl group; and [symbol]
represents the point of attachment to Y.

10. (canceled)

11. The compound of claim 1, or a pharmaceutically acceptable salt thereof, wherein A-U is 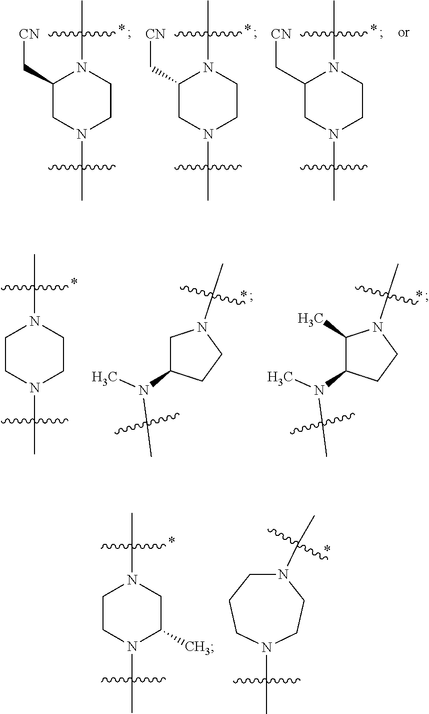
wherein [symbol] * represents the point of attachment to the carbonyl group; and [symbol]
represents the point of attachment to Y.

12. The compound of claim 1, or a pharmaceutically acceptable salt thereof, wherein R² is hydrogen or methoxy.

13. The compound of claim 1, or a pharmaceutically acceptable salt thereof, wherein R³ is halo.

14. The compound of claim 1, or a pharmaceutically acceptable salt thereof, wherein R⁴ is selected from imidazolyl, isothiazolyl, isoxazolyl, oxazolyl, phenyl, pyrazinyl, pyridazinyl, pyridinyl, pyrimidinyl, pyrazolyl, thiazolyl, thiadiazolyl, and triazolyl, wherein each ring is optionally substituted with one, two, or three groups independently selected from C₂-C₄alkenyl, C₁-C₃alkyl, halo, halo C₁-C₃alkoxy, halo C₁-C₃alkyl, nitro, and oxo.

15. The compound of claim 1, or a pharmaceutically acceptable salt thereof, wherein R⁴ is selected from imidazolyl, oxazolyl, pyridazinyl, pyridinyl, pyrimidinyl, and thiazolyl, wherein each ring is optionally substituted with a methyl or halo.

16. The compound of claim 1, or a pharmaceutically acceptable salt thereof, wherein X is O.

17. The compound of claim 1, or a pharmaceutically acceptable salt thereof, wherein R⁵ is selected from: 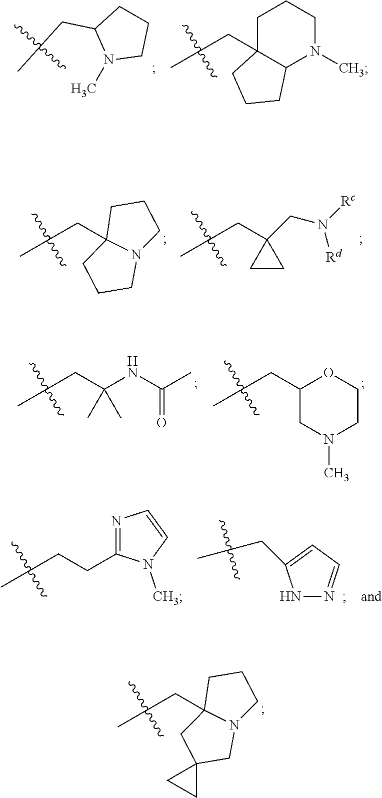
wherein each ring is optionally substituted with 1, 2, or 3 groups independently selected from C₁-C₃alkoxy, C₁-C₃alkoxy C₁-C₃alkyl, C₁-C₃alkyl, benzyl, halo, halo C₁-C₃alkyl, hydroxy, hydroxy C₁-C₃alkyl, and oxo.

18. The compound of claim 1, or a pharmaceutically acceptable salt thereof, wherein R⁵ is —(C₁-C₃alkyl)-R⁶, wherein R⁶ is a three- to five-membered monocyclic ring system, an eight- or nine-membered bicyclic fused saturated ring system, or a ten-membered tricyclic saturated ring system, wherein each ring system optionally contains one nitrogen atom, and wherein each ring system is optionally substituted with one or two groups independently selected from C₁-C₃alkyl, halo, and (4- to 6-membered heterocyclyl)C₁-C₃alkyl; wherein the heterocyclyl part of the (4- to 6-membered heterocyclyl)C₁-C₃alkyl is further optionally substituted with a halo group.

19. The compound of claim 1, or a pharmaceutically acceptable salt thereof, wherein R⁵ is 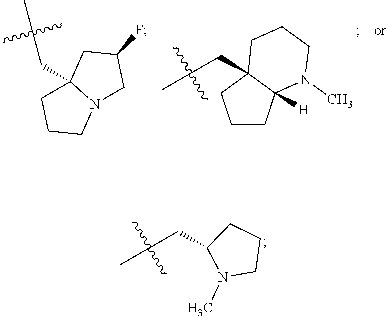
and [symbol] represents the point of attachment to X.

20. The compound of claim 1, or a pharmaceutically acceptable salt thereof, wherein R⁵ is
wherein n is 0, 1, or 2; 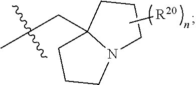
each R²⁰ is halo; and [symbol]
represents the point of attachment to X.

21. The compound of claim 1, or a pharmaceutically acceptable salt thereof, wherein Z is a bond.

22. (canceled)

23. (canceled)

24. (canceled)

25. The compound of claim 1, or a pharmaceutically acceptable salt thereof, wherein R¹ is C₆-C₁₀aryl optionally substituted with one, two, three, four, or five substituents independently selected from C₁-C₃alkoxy, C₁-C₃alkyl, C₂-C₄alkenyl, C₂-C₄alkynyl, amino, amino C₁-C₃alkyl, cyano, C₃-C₄cycloalkyl, halo, halo C₁-C₃alkyl, hydroxy, and hydroxy C₁-C₃alkyl.

26. The compound of claim 1, or a pharmaceutically acceptable salt thereof, wherein R¹ is naphthyl substituted with one, two, three, four, or five substituents independently selected from C₁-C₃alkyl, C₂-C₄alkynyl, halo, and hydroxy.

27. The compound of claim 1, or a pharmaceutically acceptable salt thereof, wherein R¹ is naphthyl, wherein the naphthyl is substituted with one, two, or three groups independently selected from C₂-C₄alkynyl, halo, and hydroxy.

28. (canceled)

29. The compound of claim 1, or a pharmaceutically acceptable salt thereof, wherein R¹ is 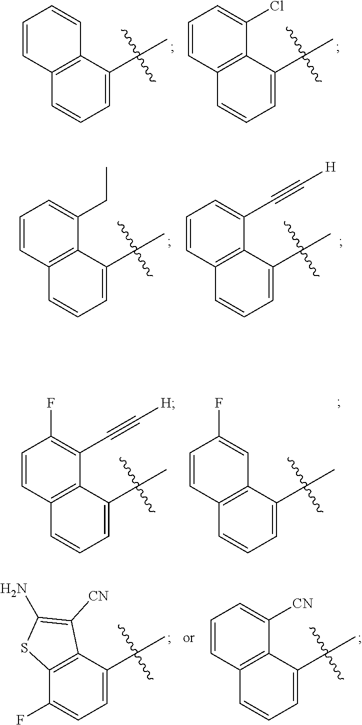
wherein [symbol] denotes the point of attachment to the parent molecular moiety.

30. The compound of claim 1, or a pharmaceutically acceptable salt thereof, wherein R′ is fluoro.

31. (canceled)

32. A compound selected from the group consisting of: 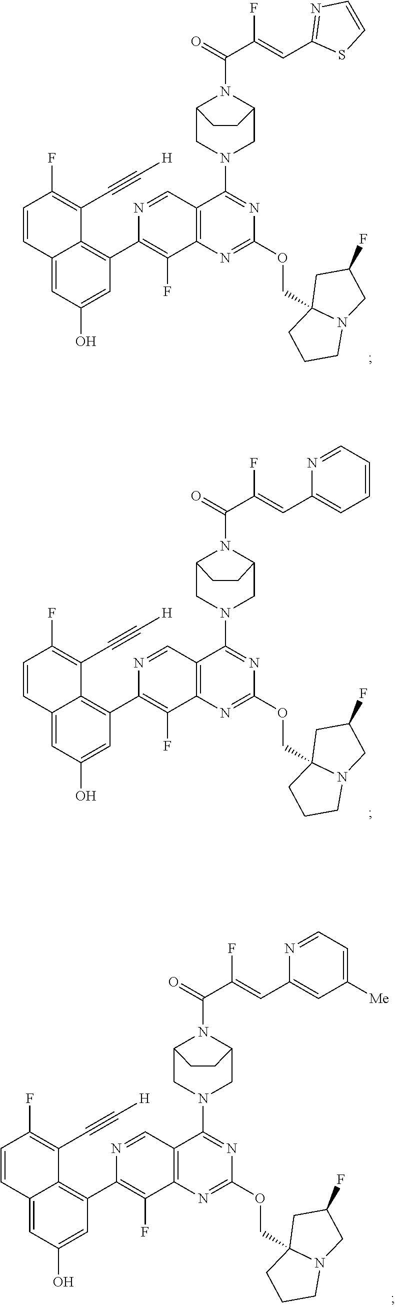 [SMILES: C#Cc1c(F)ccc2cc(O)cc(-c3ncc4c(N5CC6CCC(C5)N6C(=O)/C(F)=C/c5nccs5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5ccccn5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5cc(C)ccn5)nc(OC[C@@]56CCCN5C[C@H](F)C6)nc4c3F)c12] 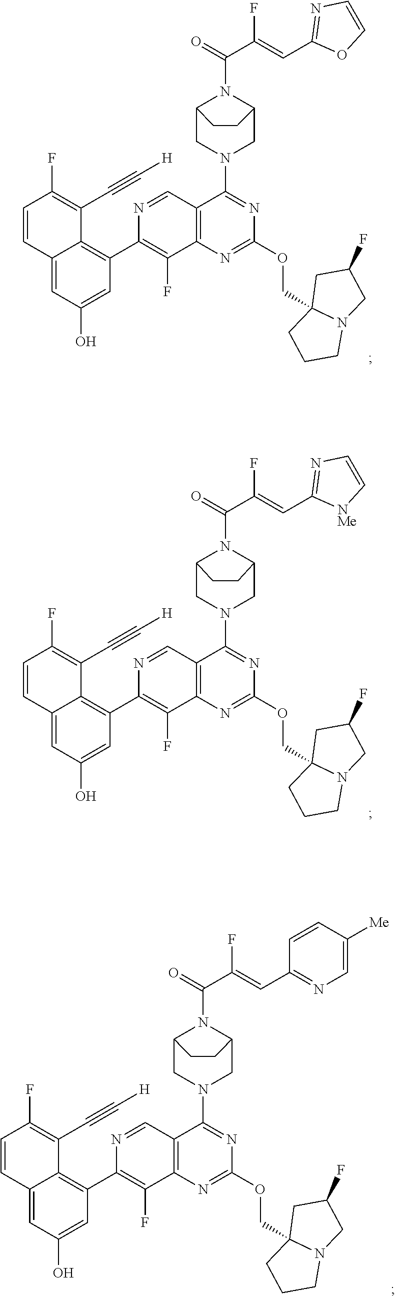 [SMILES: C#Cc1c(F)ccc2cc(O)cc(-c3ncc4c(N5CC6CCC(C5)N6C(=O)/C(F)=C/c5ncco5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5nccn5C)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5ccc(C)cn5)nc(OC[C@@]56CCCN5C[C@H](F)C6)nc4c3F)c12] 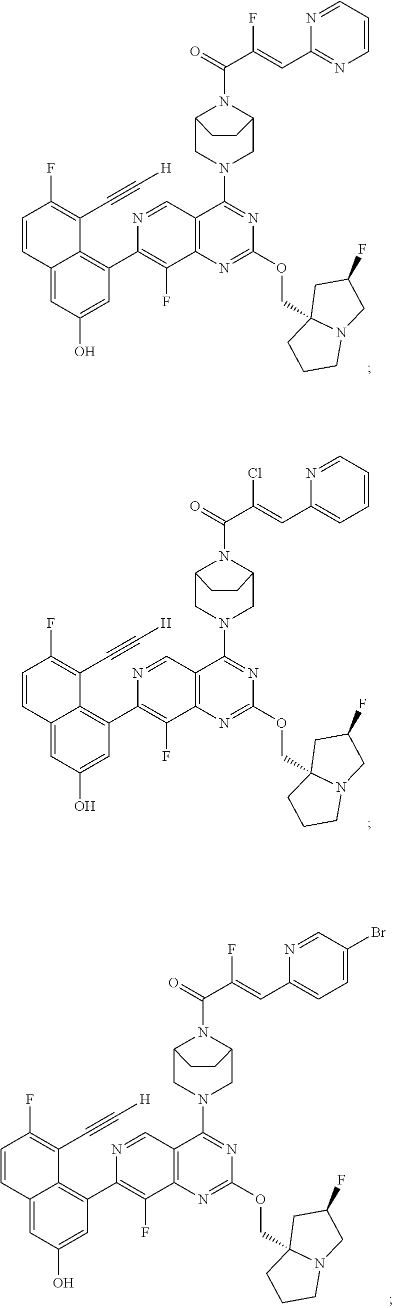 [SMILES: C#Cc1c(F)ccc2cc(O)cc(-c3ncc4c(N5CC6CCC(C5)N6C(=O)/C(F)=C/c5ncccn5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(Cl)=C/c5ccccn5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5ccc(Br)cn5)nc(OC[C@@]56CCCN5C[C@H](F)C6)nc4c3F)c12] 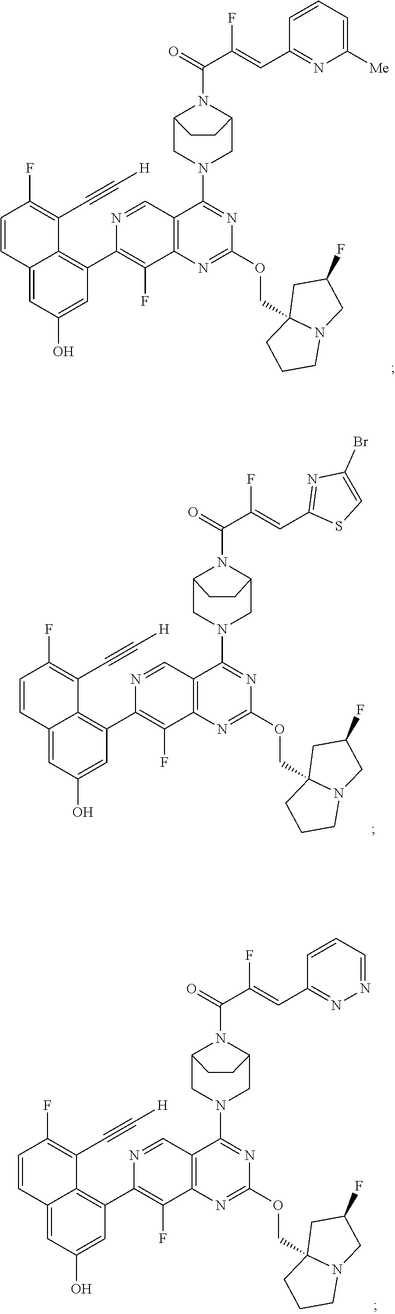 [SMILES: C#Cc1c(F)ccc2cc(O)cc(-c3ncc4c(N5CC6CCC(C5)N6C(=O)/C(F)=C/c5cccc(C)n5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5nc(Br)cs5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5cccnn5)nc(OC[C@@]56CCCN5C[C@H](F)C6)nc4c3F)c12] 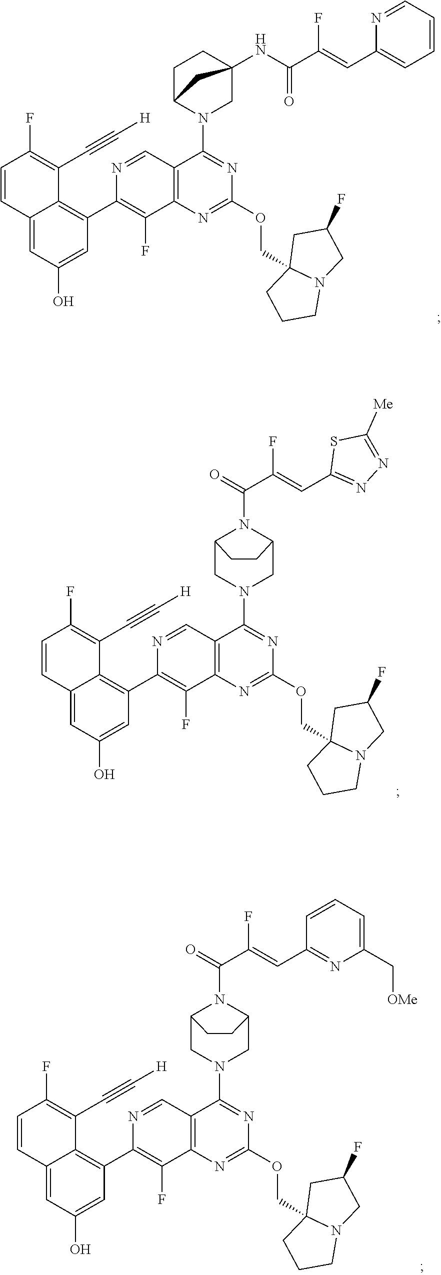 [SMILES: C#Cc1c(F)ccc2cc(O)cc(-c3ncc4c(N5C[C@@]6(NC(=O)/C(F)=C/c7ccccn7)CC[C@@H]5C6)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5nnc(C)s5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5cccc(COC)n5)nc(OC[C@@]56CCCN5C[C@H](F)C6)nc4c3F)c12] 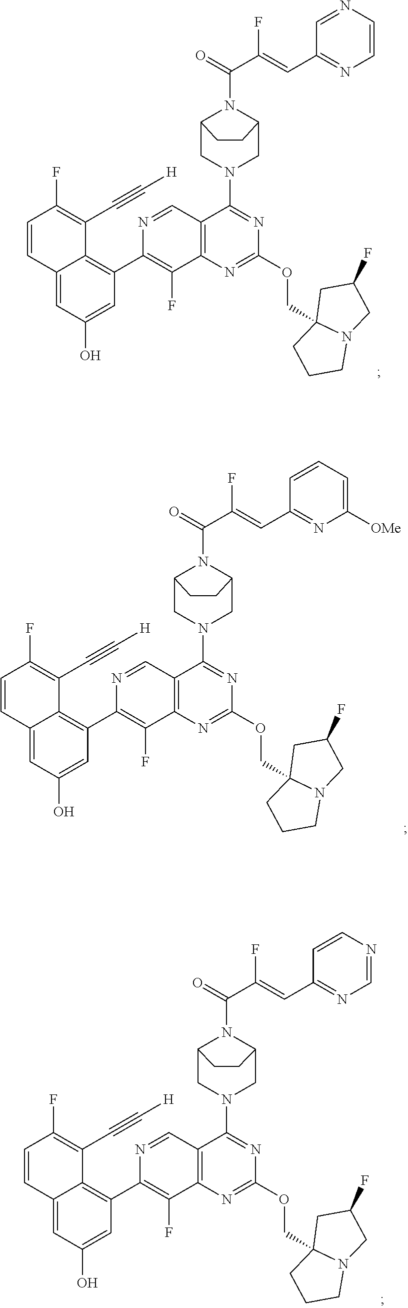 [SMILES: C#Cc1c(F)ccc2cc(O)cc(-c3ncc4c(N5CC6CCC(C5)N6C(=O)/C(F)=C/c5cnccn5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5cccc(OC)n5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)/C(F)=C/c5ccncn5)nc(OC[C@@]56CCCN5C[C@H](F)C6)nc4c3F)c12] 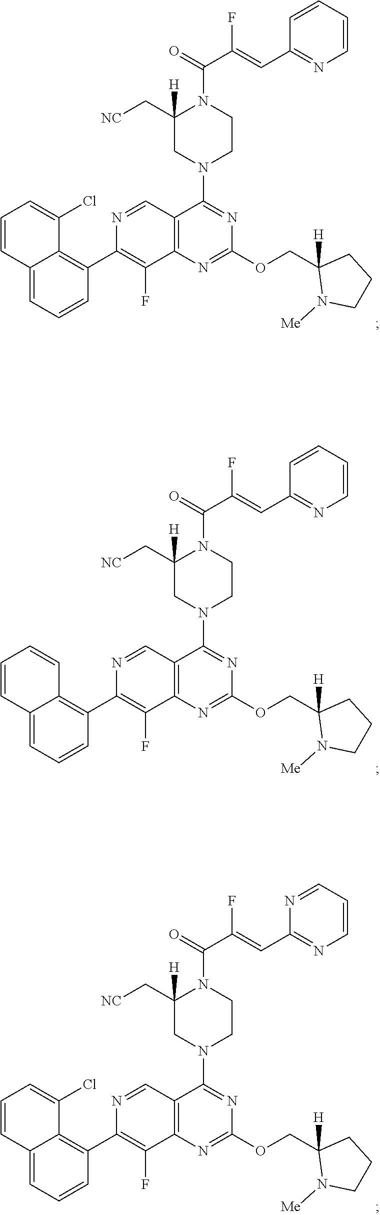 [SMILES: CN1CCC[C@H]1COc1nc(N2CCN(C(=O)/C(F)=C/c3ccccn3)[C@@H](CC#N)C2)c2cnc(-c3cccc4cccc(Cl)c34)c(F)c2n1 ; CN1CCC[C@H]1COc1nc(N2CCN(C(=O)/C(F)=C/c3ccccn3)[C@@H](CC#N)C2)c2cnc(-c3cccc4ccccc34)c(F)c2n1 ; CN1CCC[C@H]1COc1nc(N2CCN(C(=O)/C(F)=C/c3ncccn3)[C@@H](CC#N)C2)c2cnc(-c3cccc4cccc(Cl)c34)c(F)c2n1] 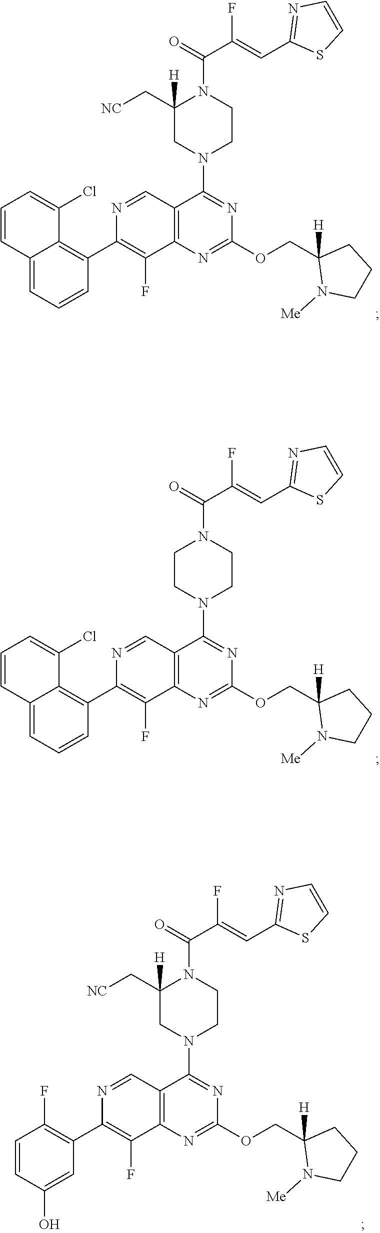 [SMILES: CN1CCC[C@H]1COc1nc(N2CCN(C(=O)/C(F)=C/c3nccs3)[C@@H](CC#N)C2)c2cnc(-c3cccc4cccc(Cl)c34)c(F)c2n1 ; CN1CCC[C@H]1COc1nc(N2CCN(C(=O)/C(F)=C/c3nccs3)CC2)c2cnc(-c3cccc4cccc(Cl)c34)c(F)c2n1 ; CN1CCC[C@H]1COc1nc(N2CCN(C(=O)/C(F)=C/c3nccs3)[C@@H](CC#N)C2)c2cnc(-c3cc(O)ccc3F)c(F)c2n1] 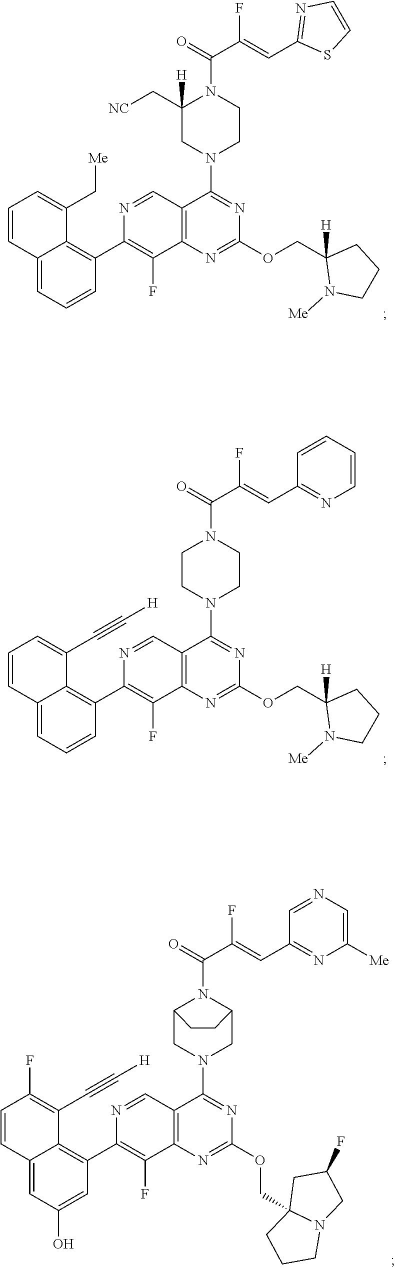 [SMILES: CCc1cccc2cccc(-c3ncc4c(N5CCN(C(=O)/C(F)=C/c6nccs6)[C@@H](CC#N)C5)nc(OC[C@@H]5CCCN5C)nc4c3F)c12 ; C#Cc1cccc2cccc(-c3ncc4c(N5CCN(C(=O)/C(F)=C/c6ccccn6)CC5)nc(OC[C@@H]5CCCN5C)nc4c3F)c12 ; C#Cc1c(F)ccc2cc(O)cc(-c3ncc4c(N5CC6CCC(C5)N6C(=O)/C(F)=C/c5cncc(C)n5)nc(OC[C@@]56CCCN5C[C@H](F)C6)nc4c3F)c12] 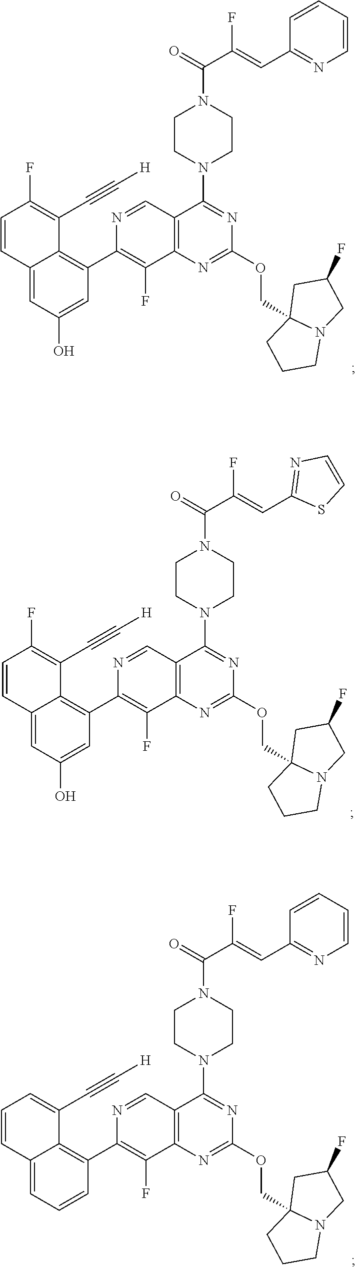 [SMILES: C#Cc1c(F)ccc2cc(O)cc(-c3ncc4c(N5CCN(C(=O)/C(F)=C/c6ccccn6)CC5)nc(OC[C@@]56CCCN5C[C@H](F)C6)nc4c3F)c12 ; C#Cc1c(F)ccc2cc(O)cc(-c3ncc4c(N5CCN(C(=O)/C(F)=C/c6nccs6)CC5)nc(OC[C@@]56CCCN5C[C@H](F)C6)nc4c3F)c12 ; C#Cc1cccc2cccc(-c3ncc4c(N5CCN(C(=O)/C(F)=C/c6ccccn6)CC5)nc(OC[C@@]56CCCN5C[C@H](F)C6)nc4c3F)c12] 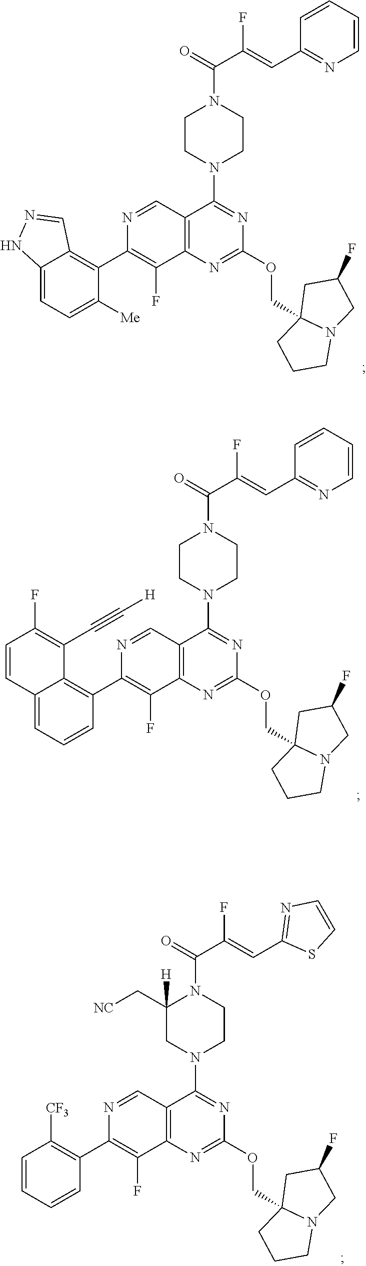 [SMILES: Cc1ccc2[nH]ncc2c1-c1ncc2c(N3CCN(C(=O)/C(F)=C/c4ccccn4)CC3)nc(OC[C@@]34CCCN3C[C@H](F)C4)nc2c1F ; C#Cc1c(F)ccc2cccc(-c3ncc4c(N5CCN(C(=O)/C(F)=C/c6ccccn6)CC5)nc(OC[C@@]56CCCN5C[C@H](F)C6)nc4c3F)c12 ; N#CC[C@H]1CN(c2nc(OC[C@@]34CCCN3C[C@H](F)C4)nc3c(F)c(-c4ccccc4C(F)(F)F)ncc23)CCN1C(=O)/C(F)=C/c1nccs1] 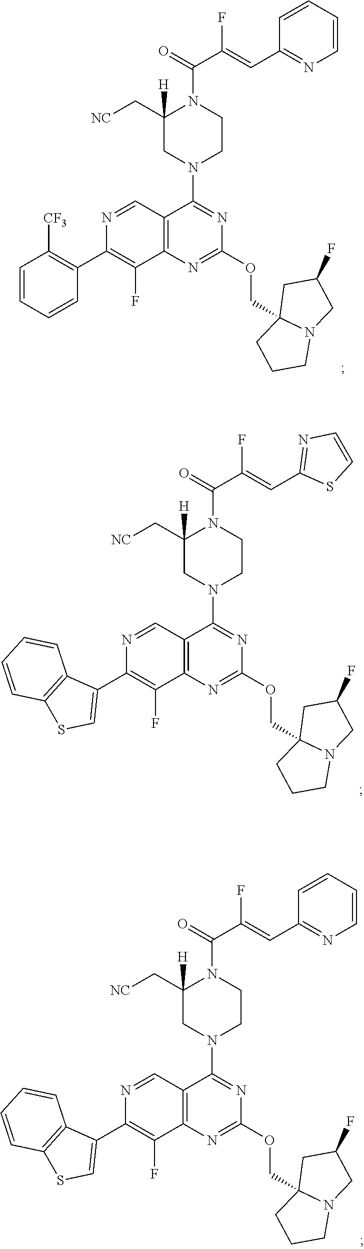 [SMILES: N#CC[C@H]1CN(c2nc(OC[C@@]34CCCN3C[C@H](F)C4)nc3c(F)c(-c4ccccc4C(F)(F)F)ncc23)CCN1C(=O)/C(F)=C/c1ccccn1 ; N#CC[C@H]1CN(c2nc(OC[C@@]34CCCN3C[C@H](F)C4)nc3c(F)c(-c4csc5ccccc45)ncc23)CCN1C(=O)/C(F)=C/c1nccs1 ; N#CC[C@H]1CN(c2nc(OC[C@@]34CCCN3C[C@H](F)C4)nc3c(F)c(-c4csc5ccccc45)ncc23)CCN1C(=O)/C(F)=C/c1ccccn1] 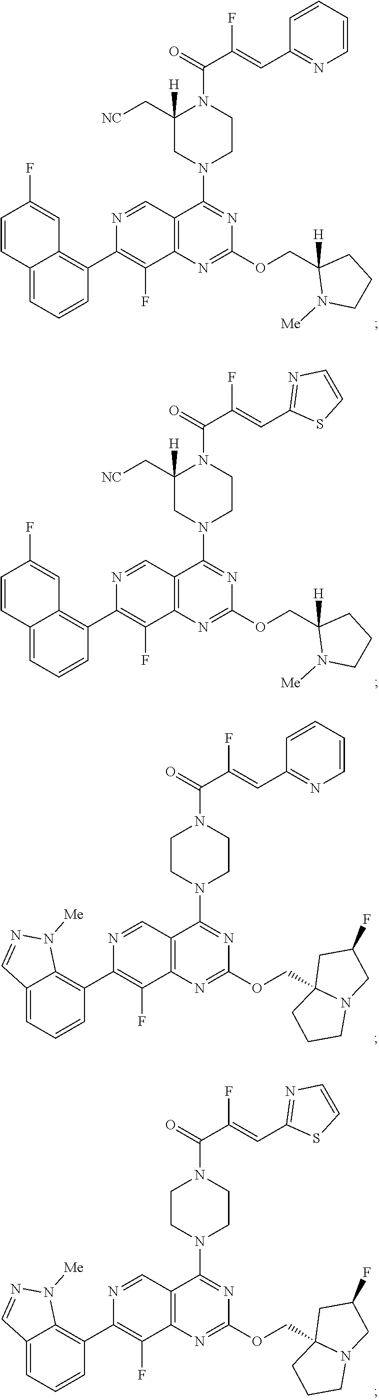 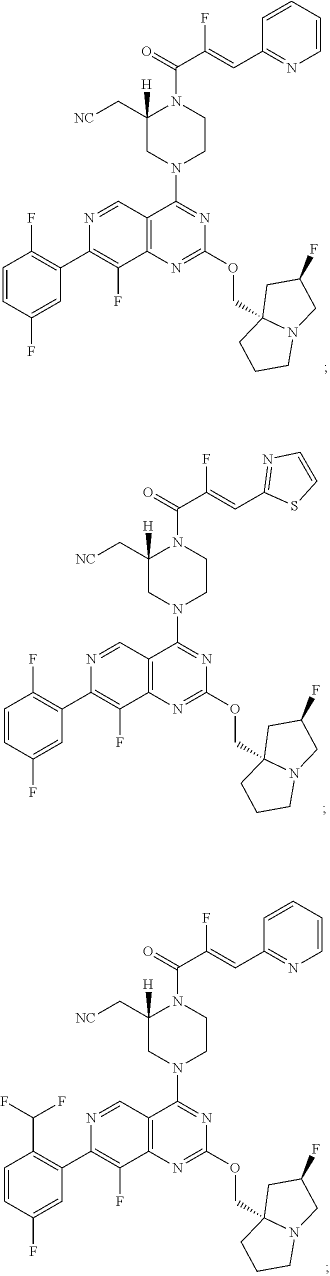 [SMILES: N#CC[C@H]1CN(c2nc(OC[C@@]34CCCN3C[C@H](F)C4)nc3c(F)c(-c4cc(F)ccc4F)ncc23)CCN1C(=O)/C(F)=C/c1ccccn1 ; N#CC[C@H]1CN(c2nc(OC[C@@]34CCCN3C[C@H](F)C4)nc3c(F)c(-c4cc(F)ccc4F)ncc23)CCN1C(=O)/C(F)=C/c1nccs1 ; N#CC[C@H]1CN(c2nc(OC[C@@]34CCCN3C[C@H](F)C4)nc3c(F)c(-c4cc(F)ccc4C(F)F)ncc23)CCN1C(=O)/C(F)=C/c1ccccn1] 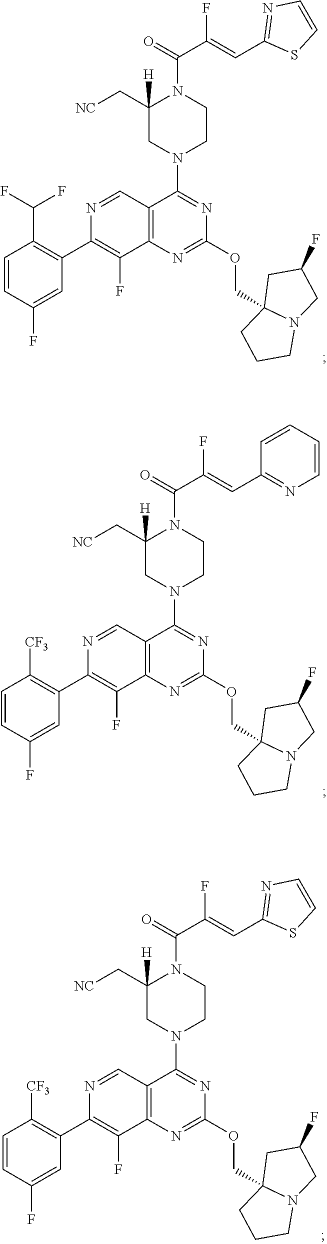 [SMILES: N#CC[C@H]1CN(c2nc(OC[C@@]34CCCN3C[C@H](F)C4)nc3c(F)c(-c4cc(F)ccc4C(F)F)ncc23)CCN1C(=O)/C(F)=C/c1nccs1 ; N#CC[C@H]1CN(c2nc(OC[C@@]34CCCN3C[C@H](F)C4)nc3c(F)c(-c4cc(F)ccc4C(F)(F)F)ncc23)CCN1C(=O)/C(F)=C/c1ccccn1 ; N#CC[C@H]1CN(c2nc(OC[C@@]34CCCN3C[C@H](F)C4)nc3c(F)c(-c4cc(F)ccc4C(F)(F)F)ncc23)CCN1C(=O)/C(F)=C/c1nccs1] 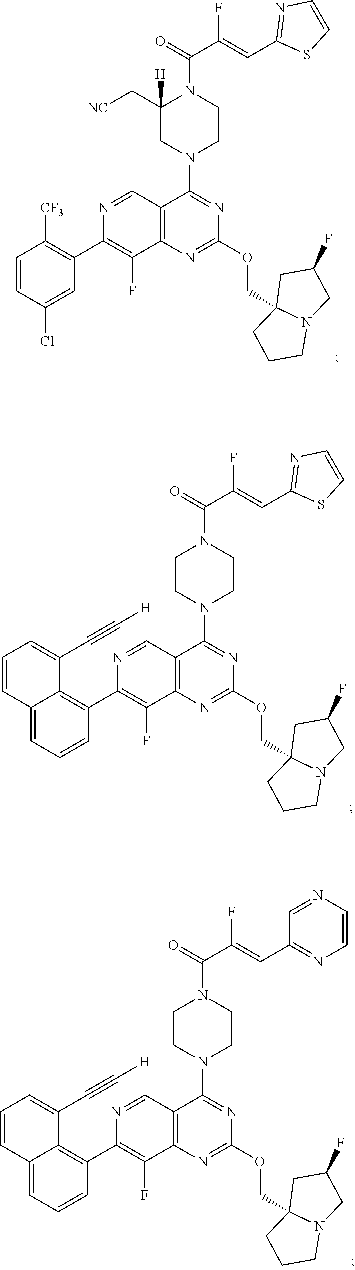 [SMILES: N#CC[C@H]1CN(c2nc(OC[C@@]34CCCN3C[C@H](F)C4)nc3c(F)c(-c4cc(Cl)ccc4C(F)(F)F)ncc23)CCN1C(=O)/C(F)=C/c1nccs1 ; C#Cc1cccc2cccc(-c3ncc4c(N5CCN(C(=O)/C(F)=C/c6nccs6)CC5)nc(OC[C@@]56CCCN5C[C@H](F)C6)nc4c3F)c12 ; C#Cc1cccc2cccc(-c3ncc4c(N5CCN(C(=O)/C(F)=C/c6cnccn6)CC5)nc(OC[C@@]56CCCN5C[C@H](F)C6)nc4c3F)c12] 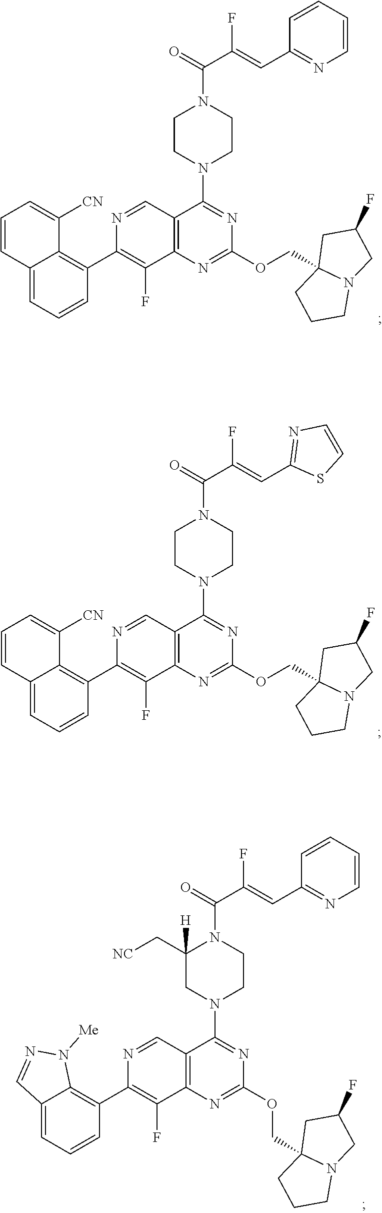 [SMILES: N#Cc1cccc2cccc(-c3ncc4c(N5CCN(C(=O)/C(F)=C/c6ccccn6)CC5)nc(OC[C@@]56CCCN5C[C@H](F)C6)nc4c3F)c12 ; N#Cc1cccc2cccc(-c3ncc4c(N5CCN(C(=O)/C(F)=C/c6nccs6)CC5)nc(OC[C@@]56CCCN5C[C@H](F)C6)nc4c3F)c12 ; Cn1ncc2cccc(-c3ncc4c(N5CCN(C(=O)/C(F)=C/c6ccccn6)[C@@H](CC#N)C5)nc(OC[C@@]56CCCN5C[C@H](F)C6)nc4c3F)c21] 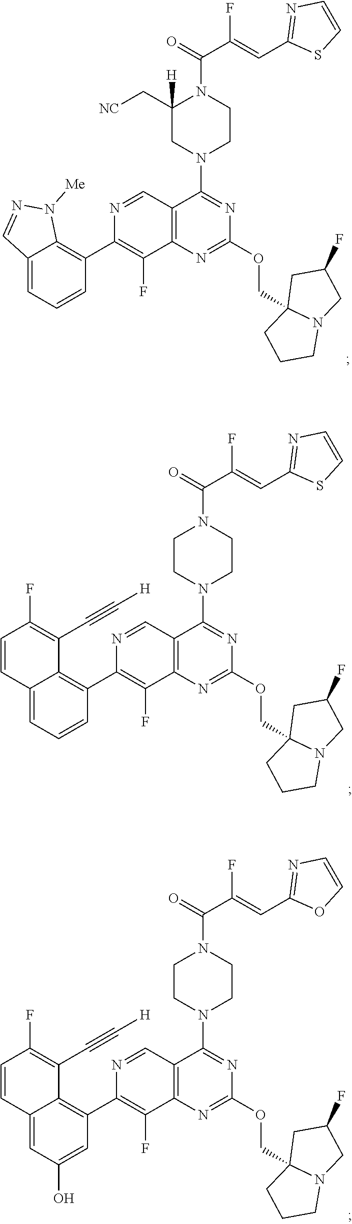 [SMILES: Cn1ncc2cccc(-c3ncc4c(N5CCN(C(=O)/C(F)=C/c6nccs6)[C@@H](CC#N)C5)nc(OC[C@@]56CCCN5C[C@H](F)C6)nc4c3F)c21 ; C#Cc1c(F)ccc2cccc(-c3ncc4c(N5CCN(C(=O)/C(F)=C/c6nccs6)CC5)nc(OC[C@@]56CCCN5C[C@H](F)C6)nc4c3F)c12 ; C#Cc1c(F)ccc2cc(O)cc(-c3ncc4c(N5CCN(C(=O)/C(F)=C/c6ncco6)CC5)nc(OC[C@@]56CCCN5C[C@H](F)C6)nc4c3F)c12] 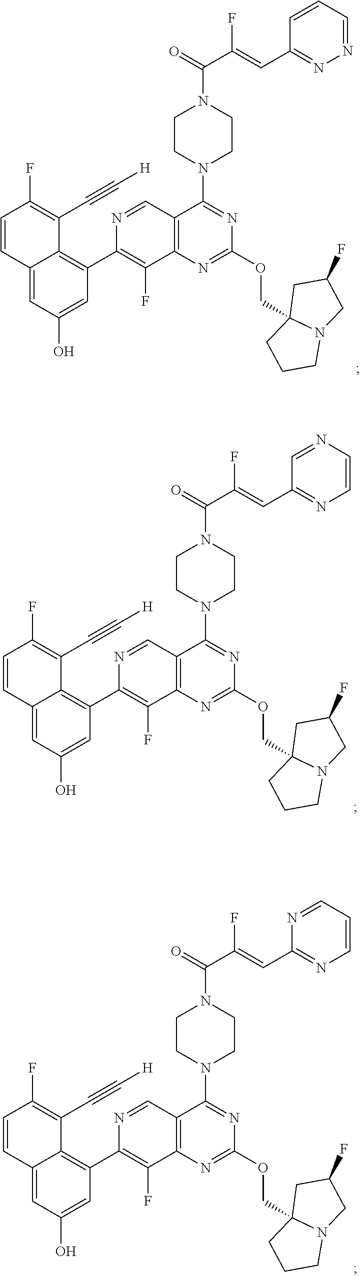 [SMILES: C#Cc1c(F)ccc2cc(O)cc(-c3ncc4c(N5CCN(C(=O)/C(F)=C/c6cccnn6)CC5)nc(OC[C@@]56CCCN5C[C@H](F)C6)nc4c3F)c12 ; C#Cc1c(F)ccc2cc(O)cc(-c3ncc4c(N5CCN(C(=O)/C(F)=C/c6cnccn6)CC5)nc(OC[C@@]56CCCN5C[C@H](F)C6)nc4c3F)c12 ; C#Cc1c(F)ccc2cc(O)cc(-c3ncc4c(N5CCN(C(=O)/C(F)=C/c6ncccn6)CC5)nc(OC[C@@]56CCCN5C[C@H](F)C6)nc4c3F)c12] 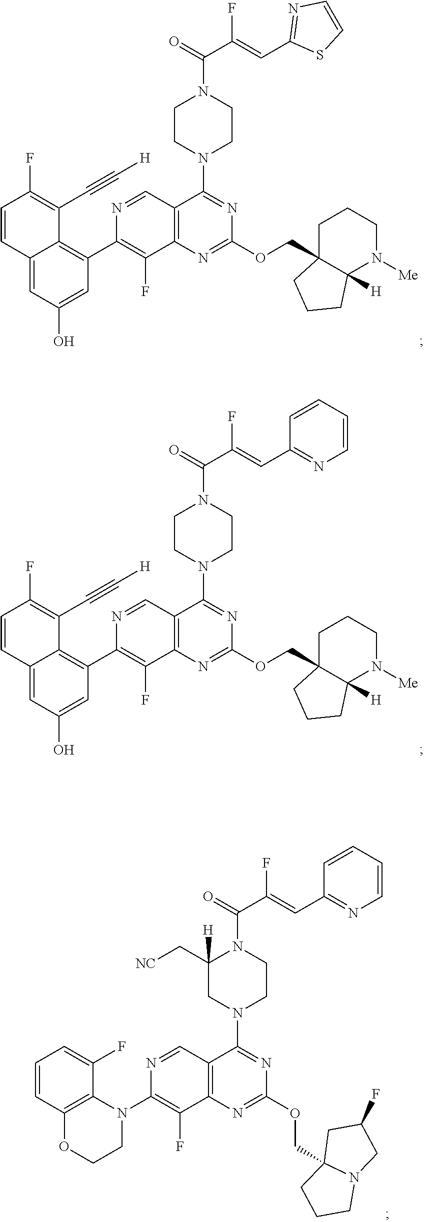 [SMILES: C#Cc1c(F)ccc2cc(O)cc(-c3ncc4c(N5CCN(C(=O)/C(F)=C/c6nccs6)CC5)nc(OC[C@]56CCC[C@H]5N(C)CCC6)nc4c3F)c12 ; C#Cc1c(F)ccc2cc(O)cc(-c3ncc4c(N5CCN(C(=O)/C(F)=C/c6ccccn6)CC5)nc(OC[C@]56CCC[C@H]5N(C)CCC6)nc4c3F)c12 ; N#CC[C@H]1CN(c2nc(OC[C@@]34CCCN3C[C@H](F)C4)nc3c(F)c(N4CCOc5cccc(F)c54)ncc23)CCN1C(=O)/C(F)=C/c1ccccn1] 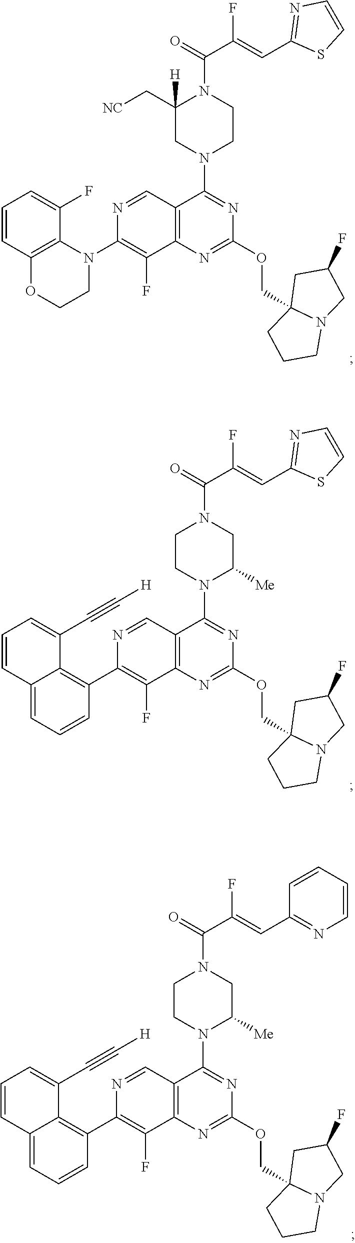 [SMILES: N#CC[C@H]1CN(c2nc(OC[C@@]34CCCN3C[C@H](F)C4)nc3c(F)c(N4CCOc5cccc(F)c54)ncc23)CCN1C(=O)/C(F)=C/c1nccs1 ; C#Cc1cccc2cccc(-c3ncc4c(N5CCN(C(=O)/C(F)=C/c6nccs6)C[C@@H]5C)nc(OC[C@@]56CCCN5C[C@H](F)C6)nc4c3F)c12 ; C#Cc1cccc2cccc(-c3ncc4c(N5CCN(C(=O)/C(F)=C/c6ccccn6)C[C@@H]5C)nc(OC[C@@]56CCCN5C[C@H](F)C6)nc4c3F)c12] 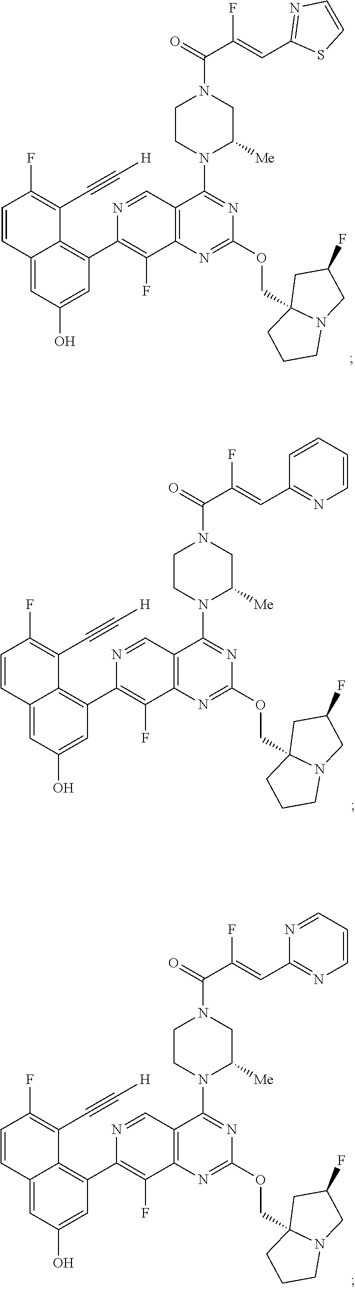 [SMILES: C#Cc1c(F)ccc2cc(O)cc(-c3ncc4c(N5CCN(C(=O)/C(F)=C/c6nccs6)C[C@@H]5C)nc(OC[C@@]56CCCN5C[C@H](F)C6)nc4c3F)c12 ; C#Cc1c(F)ccc2cc(O)cc(-c3ncc4c(N5CCN(C(=O)/C(F)=C/c6ccccn6)C[C@@H]5C)nc(OC[C@@]56CCCN5C[C@H](F)C6)nc4c3F)c12 ; C#Cc1c(F)ccc2cc(O)cc(-c3ncc4c(N5CCN(C(=O)/C(F)=C/c6ncccn6)C[C@@H]5C)nc(OC[C@@]56CCCN5C[C@H](F)C6)nc4c3F)c12] 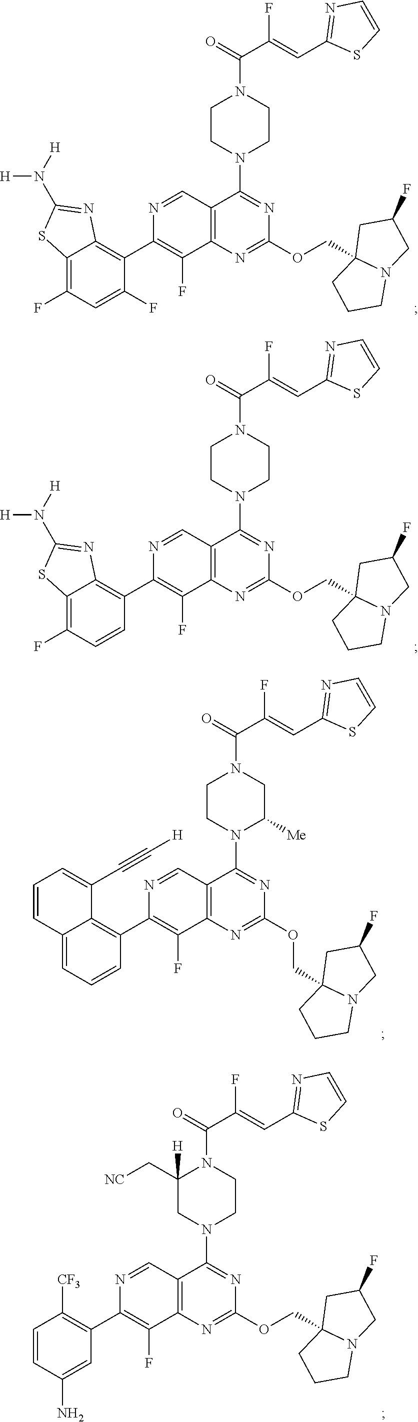 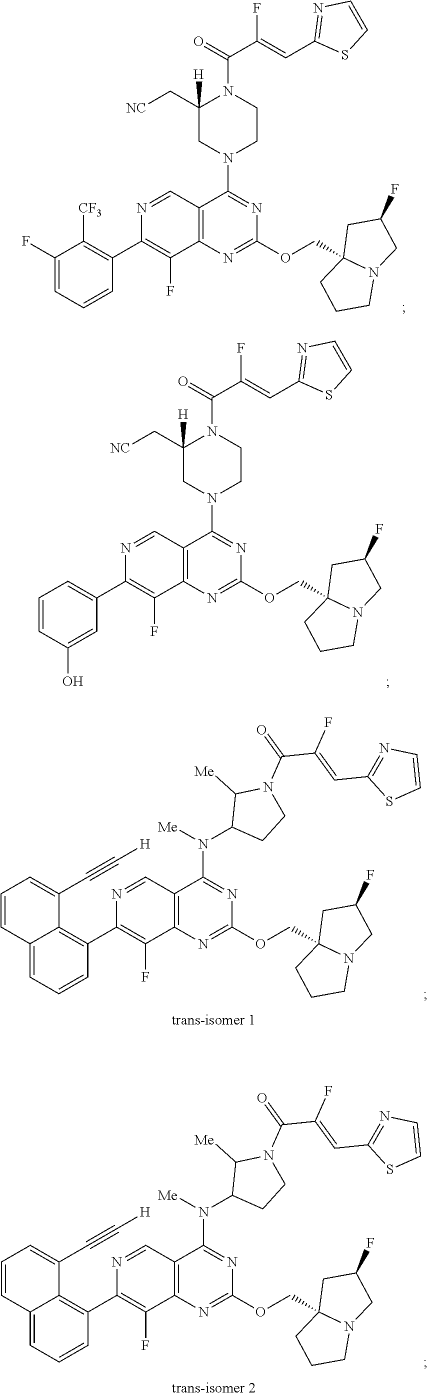 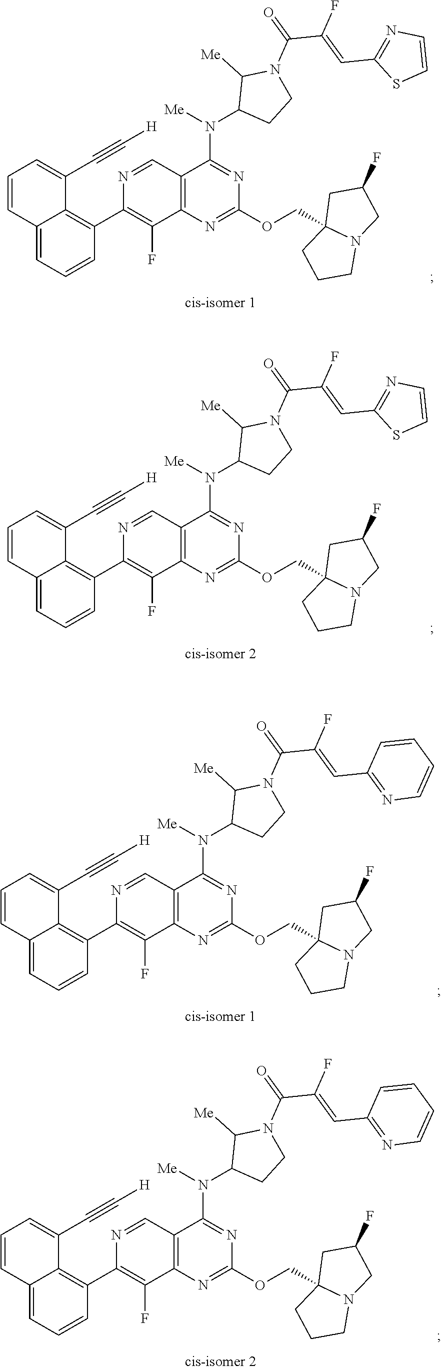 [SMILES: C#Cc1cccc2cccc(-c3ncc4c(N(C)C5CCN(C(=O)/C(F)=C/c6nccs6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6nccs6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6ccccn6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6ccccn6)C5C)nc(OC[C@@]56CCCN5C[C@H](F)C6)nc4c3F)c12] 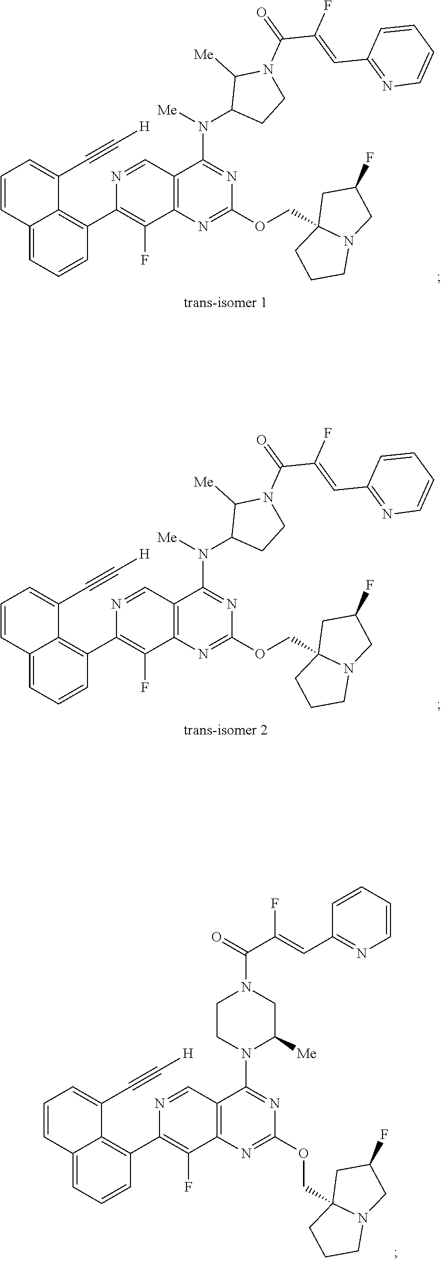 [SMILES: C#Cc1cccc2cccc(-c3ncc4c(N(C)C5CCN(C(=O)/C(F)=C/c6ccccn6)C5C)nc(OC[C@@]56CCCN5C[C@H](F)C6)nc4c3F)c12 ; C#Cc1cccc2cccc(-c3ncc4c(N(C)C5CCN(C(=O)/C(F)=C/c6ccccn6)C5C)nc(OC[C@@]56CCCN5C[C@H](F)C6)nc4c3F)c12 ; C#Cc1cccc2cccc(-c3ncc4c(N5CCN(C(=O)/C(F)=C/c6ccccn6)C[C@H]5C)nc(OC[C@@]56CCCN5C[C@H](F)C6)nc4c3F)c12] 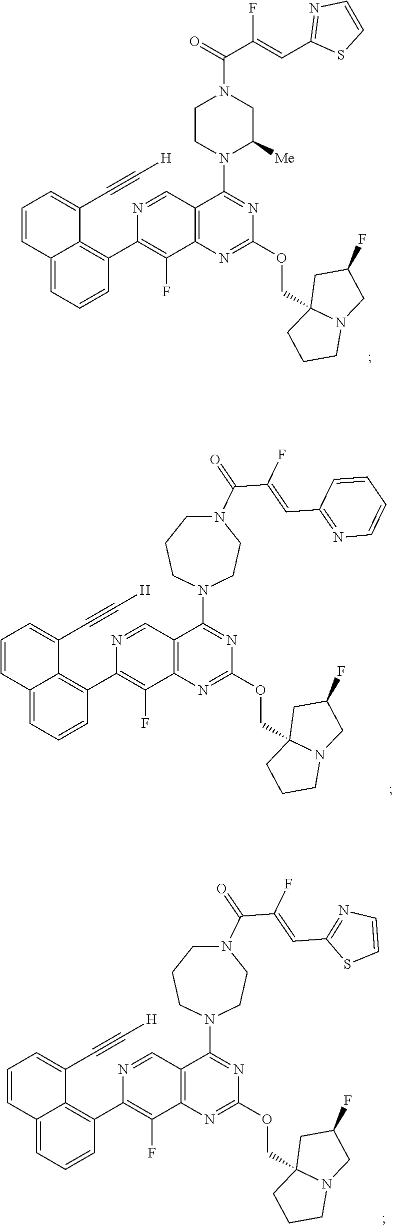 [SMILES: C#Cc1cccc2cccc(-c3ncc4c(N5CCN(C(=O)/C(F)=C/c6nccs6)C[C@H]5C)nc(OC[C@@]56CCCN5C[C@H](F)C6)nc4c3F)c12 ; C#Cc1cccc2cccc(-c3ncc4c(N5CCCN(C(=O)/C(F)=C/c6ccccn6)CC5)nc(OC[C@@]56CCCN5C[C@H](F)C6)nc4c3F)c12 ; C#Cc1cccc2cccc(-c3ncc4c(N5CCCN(C(=O)/C(F)=C/c6nccs6)CC5)nc(OC[C@@]56CCCN5C[C@H](F)C6)nc4c3F)c12] 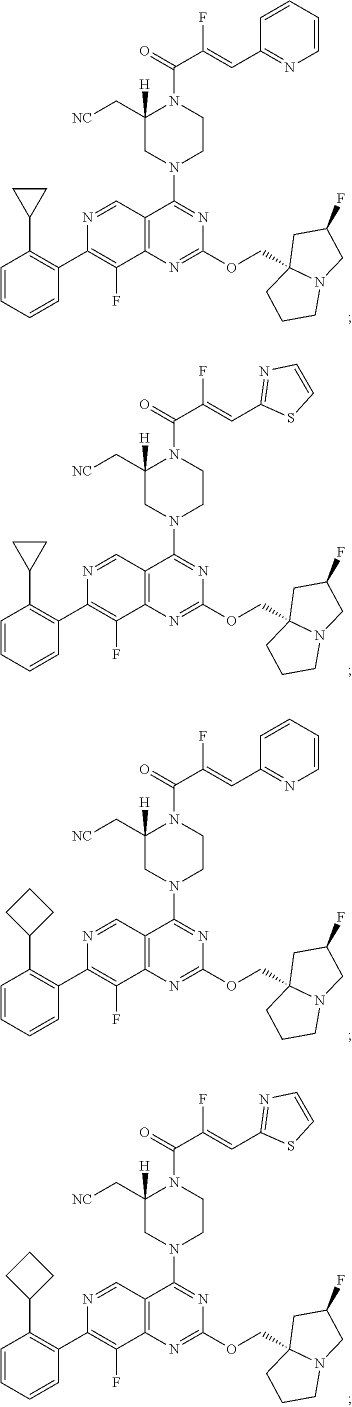 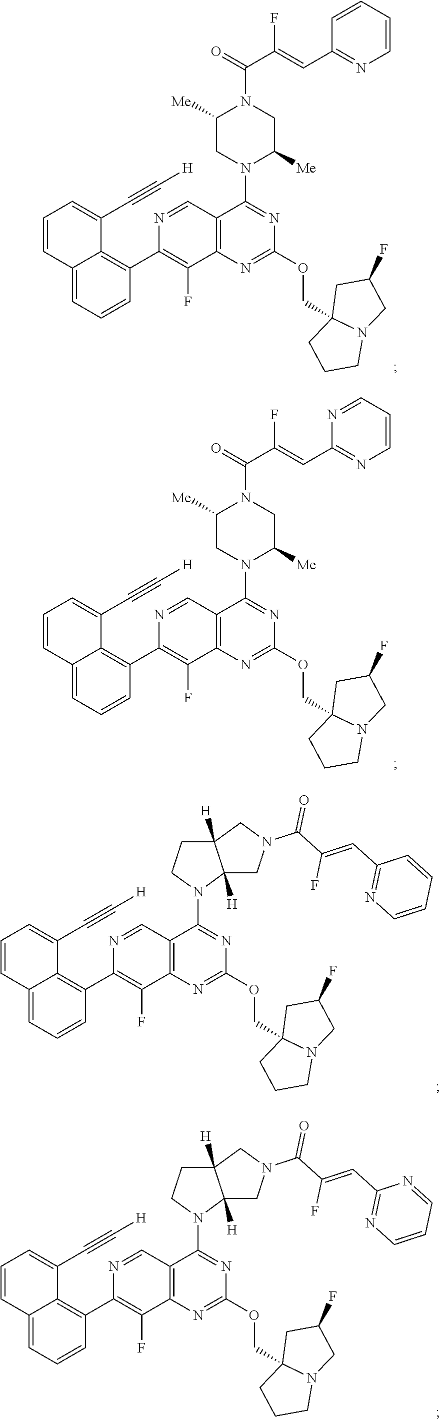 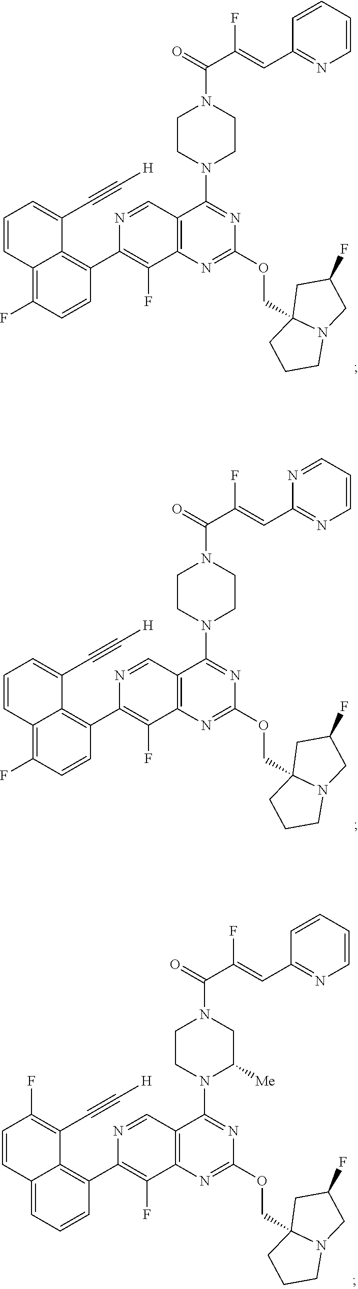 [SMILES: C#Cc1cccc2c(F)ccc(-c3ncc4c(N5CCN(C(=O)/C(F)=C/c6ccccn6)CC5)nc(OC[C@@]56CCCN5C[C@H](F)C6)nc4c3F)c12 ; C#Cc1cccc2c(F)ccc(-c3ncc4c(N5CCN(C(=O)/C(F)=C/c6ncccn6)CC5)nc(OC[C@@]56CCCN5C[C@H](F)C6)nc4c3F)c12 ; C#Cc1c(F)ccc2cccc(-c3ncc4c(N5CCN(C(=O)/C(F)=C/c6ccccn6)C[C@@H]5C)nc(OC[C@@]56CCCN5C[C@H](F)C6)nc4c3F)c12] 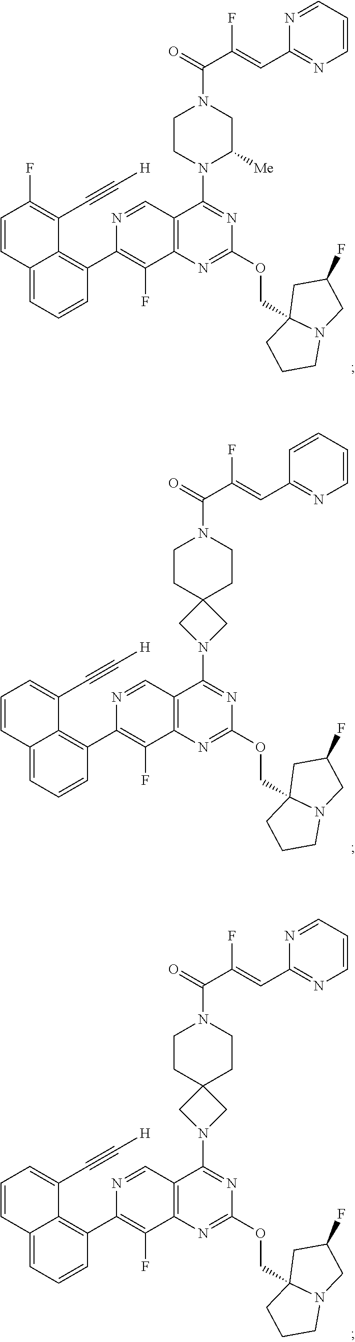 [SMILES: C#Cc1c(F)ccc2cccc(-c3ncc4c(N5CCN(C(=O)/C(F)=C/c6ncccn6)C[C@@H]5C)nc(OC[C@@]56CCCN5C[C@H](F)C6)nc4c3F)c12 ; C#Cc1cccc2cccc(-c3ncc4c(N5CC6(CCN(C(=O)/C(F)=C/c7ccccn7)CC6)C5)nc(OC[C@@]56CCCN5C[C@H](F)C6)nc4c3F)c12 ; C#Cc1cccc2cccc(-c3ncc4c(N5CC6(CCN(C(=O)/C(F)=C/c7ncccn7)CC6)C5)nc(OC[C@@]56CCCN5C[C@H](F)C6)nc4c3F)c12] 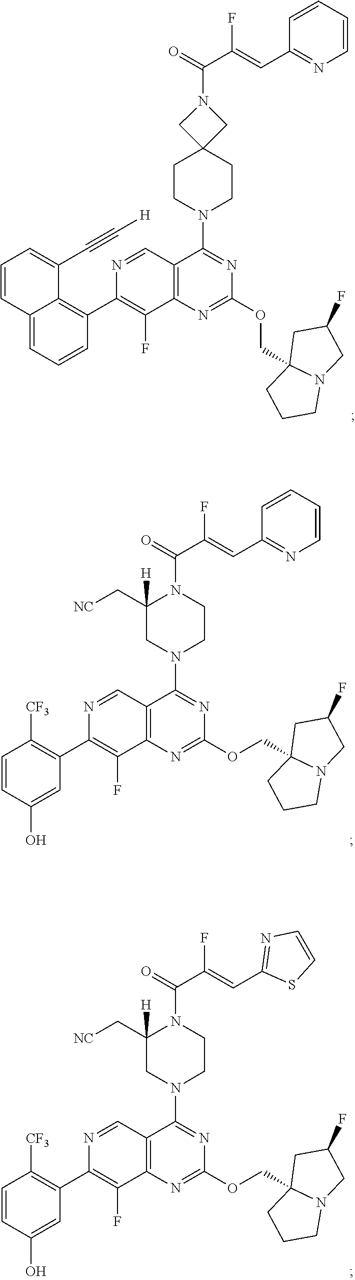 [SMILES: C#Cc1cccc2cccc(-c3ncc4c(N5CCC6(CC5)CN(C(=O)/C(F)=C/c5ccccn5)C6)nc(OC[C@@]56CCCN5C[C@H](F)C6)nc4c3F)c12 ; N#CC[C@H]1CN(c2nc(OC[C@@]34CCCN3C[C@H](F)C4)nc3c(F)c(-c4cc(O)ccc4C(F)(F)F)ncc23)CCN1C(=O)/C(F)=C/c1ccccn1 ; N#CC[C@H]1CN(c2nc(OC[C@@]34CCCN3C[C@H](F)C4)nc3c(F)c(-c4cc(O)ccc4C(F)(F)F)ncc23)CCN1C(=O)/C(F)=C/c1nccs1] 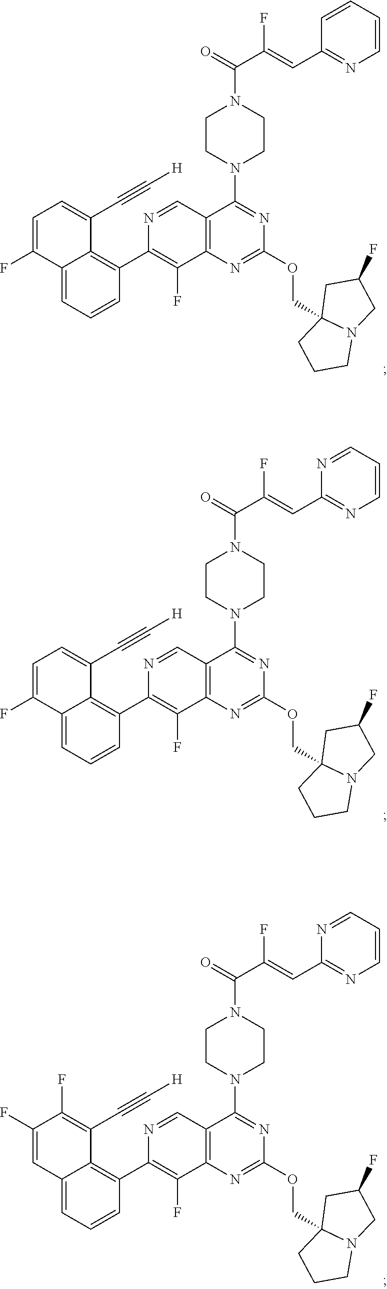 [SMILES: C#Cc1ccc(F)c2cccc(-c3ncc4c(N5CCN(C(=O)/C(F)=C/c6ccccn6)CC5)nc(OC[C@@]56CCCN5C[C@H](F)C6)nc4c3F)c12 ; C#Cc1ccc(F)c2cccc(-c3ncc4c(N5CCN(C(=O)/C(F)=C/c6ncccn6)CC5)nc(OC[C@@]56CCCN5C[C@H](F)C6)nc4c3F)c12 ; C#Cc1c(F)c(F)cc2cccc(-c3ncc4c(N5CCN(C(=O)/C(F)=C/c6ncccn6)CC5)nc(OC[C@@]56CCCN5C[C@H](F)C6)nc4c3F)c12] 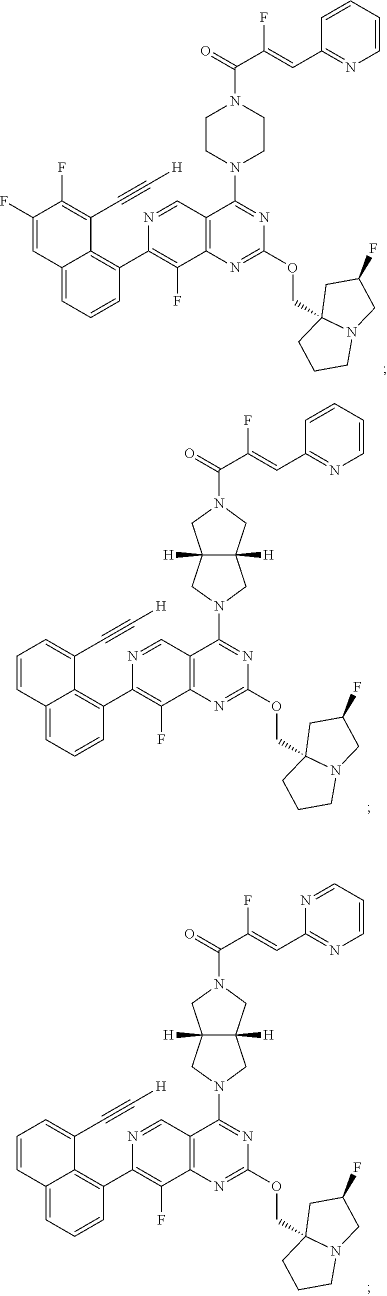 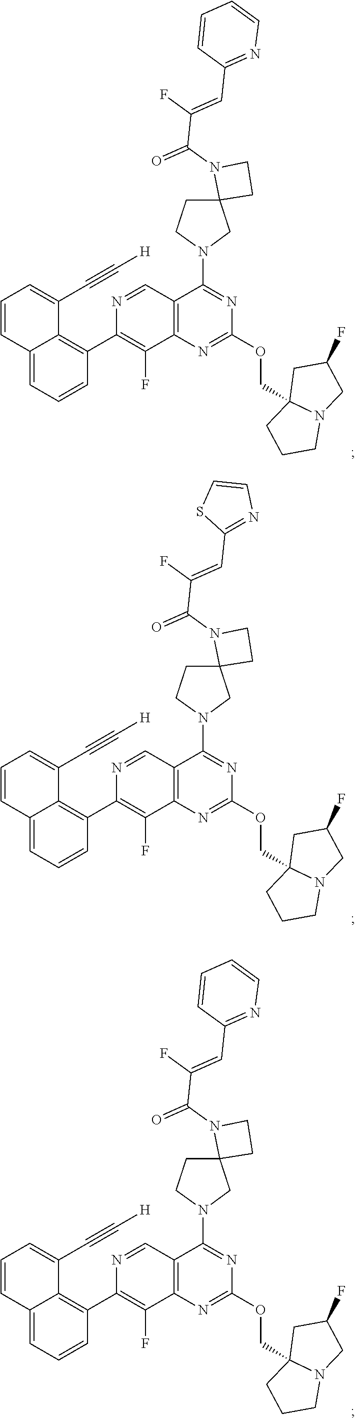 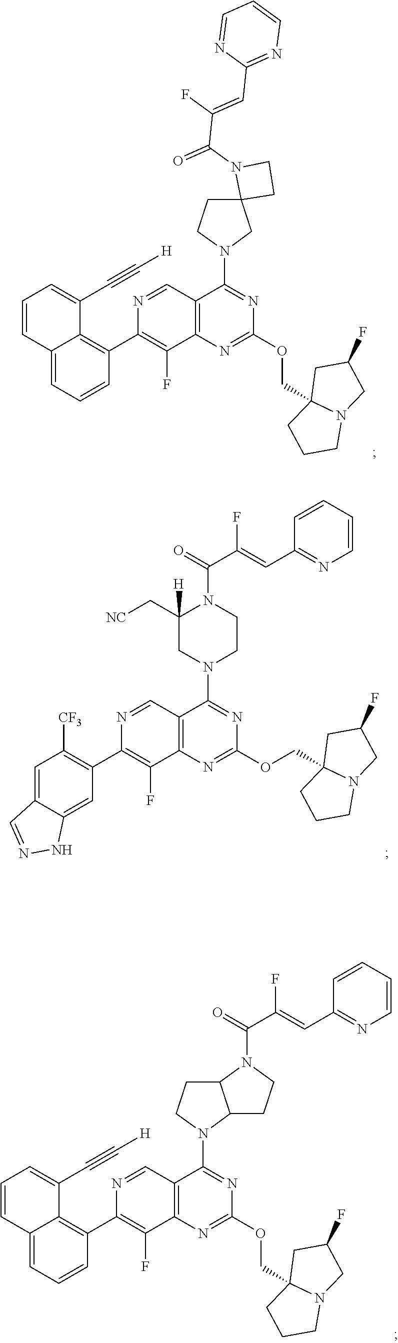 [SMILES: C#Cc1cccc2cccc(-c3ncc4c(N5CCC6(CCN6C(=O)/C(F)=C/c6ncccn6)C5)nc(OC[C@@]56CCCN5C[C@H](F)C6)nc4c3F)c12 ; N#CC[C@H]1CN(c2nc(OC[C@@]34CCCN3C[C@H](F)C4)nc3c(F)c(-c4cc5[nH]ncc5cc4C(F)(F)F)ncc23)CCN1C(=O)/C(F)=C/c1ccccn1 ; C#Cc1cccc2cccc(-c3ncc4c(N5CCC6C5CCN6C(=O)/C(F)=C/c5ccccn5)nc(OC[C@@]56CCCN5C[C@H](F)C6)nc4c3F)c12] 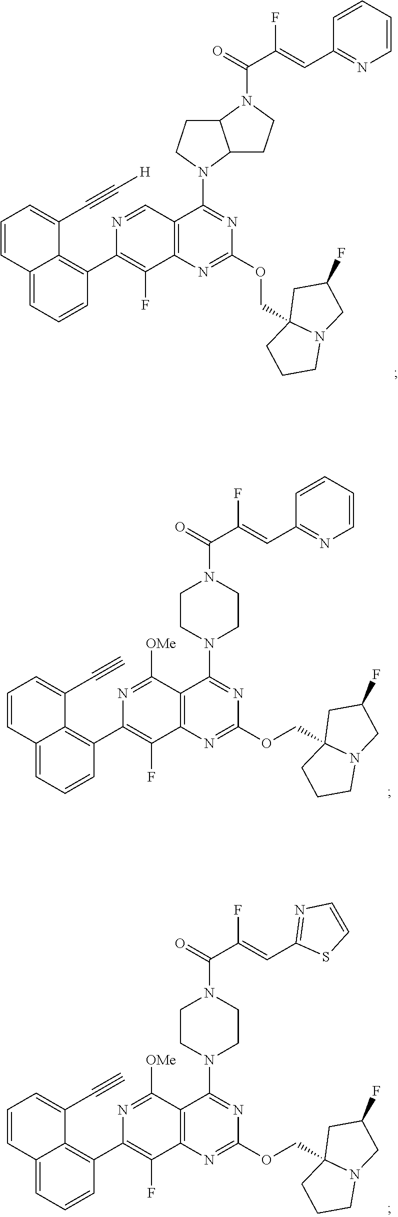 [SMILES: C#Cc1cccc2cccc(-c3ncc4c(N5CCC6C5CCN6C(=O)/C(F)=C/c5ccccn5)nc(OC[C@@]56CCCN5C[C@H](F)C6)nc4c3F)c12 ; C#Cc1cccc2cccc(-c3nc(OC)c4c(N5CCN(C(=O)/C(F)=C/c6ccccn6)CC5)nc(OC[C@@]56CCCN5C[C@H](F)C6)nc4c3F)c12 ; C#Cc1cccc2cccc(-c3nc(OC)c4c(N5CCN(C(=O)/C(F)=C/c6nccs6)CC5)nc(OC[C@@]56CCCN5C[C@H](F)C6)nc4c3F)c12] 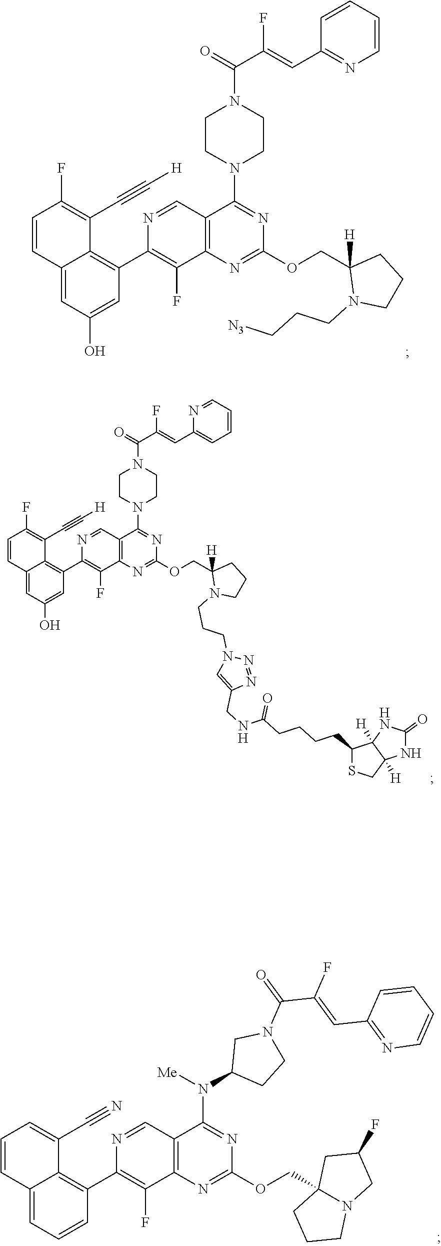 [SMILES: C#Cc1c(F)ccc2cc(O)cc(-c3ncc4c(N5CCN(C(=O)/C(F)=C/c6ccccn6)CC5)nc(OC[C@@H]5CCCN5CCCN=[N+]=[N-])nc4c3F)c12 ; C#Cc1c(F)ccc2cc(O)cc(-c3ncc4c(N5CCN(C(=O)/C(F)=C/c6ccccn6)CC5)nc(OC[C@@H]5CCCN5CCCn5cc(CNC(=O)CCCC[C@@H]6SC[C@@H]7NC(=O)N[C@@H]76)nn5)nc4c3F)c12 ; CN(c1nc(OC[C@@]23CCCN2C[C@H](F)C3)nc2c(F)c(-c3cccc4cccc(C#N)c34)ncc12)[C@@H]1CCN(C(=O)/C(F)=C/c2ccccn2)C1] 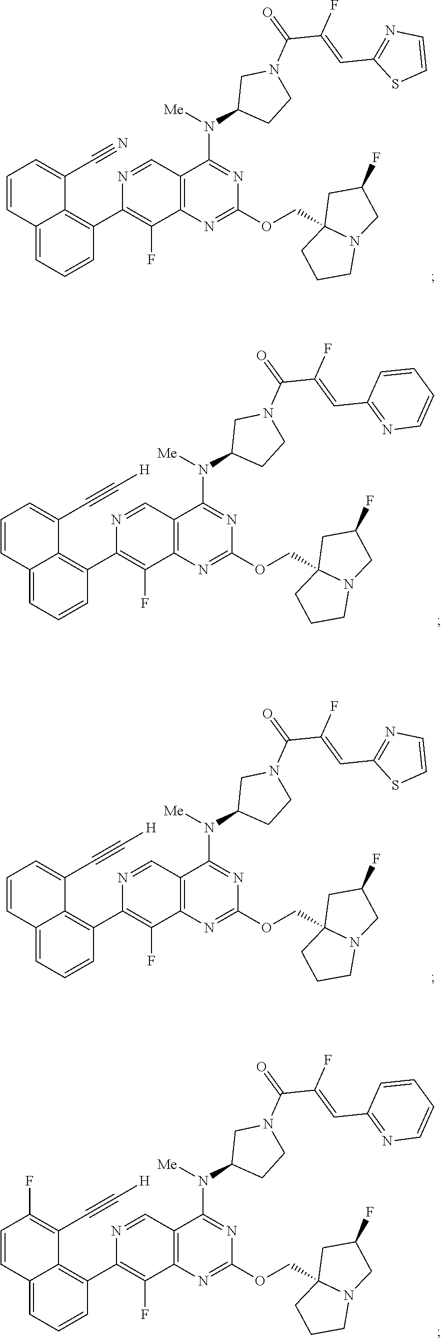 [SMILES: CN(c1nc(OC[C@@]23CCCN2C[C@H](F)C3)nc2c(F)c(-c3cccc4cccc(C#N)c34)ncc12)[C@@H]1CCN(C(=O)/C(F)=C/c2nccs2)C1 ; C#Cc1cccc2cccc(-c3ncc4c(N(C)[C@@H]5CCN(C(=O)/C(F)=C/c6ccccn6)C5)nc(OC[C@@]56CCCN5C[C@H](F)C6)nc4c3F)c12 ; C#Cc1cccc2cccc(-c3ncc4c(N(C)[C@@H]5CCN(C(=O)/C(F)=C/c6nccs6)C5)nc(OC[C@@]56CCCN5C[C@H](F)C6)nc4c3F)c12 ; C#Cc1c(F)ccc2cccc(-c3ncc4c(N(C)[C@@H]5CCN(C(=O)/C(F)=C/c6ccccn6)C5)nc(OC[C@@]56CCCN5C[C@H](F)C6)nc4c3F)c12] 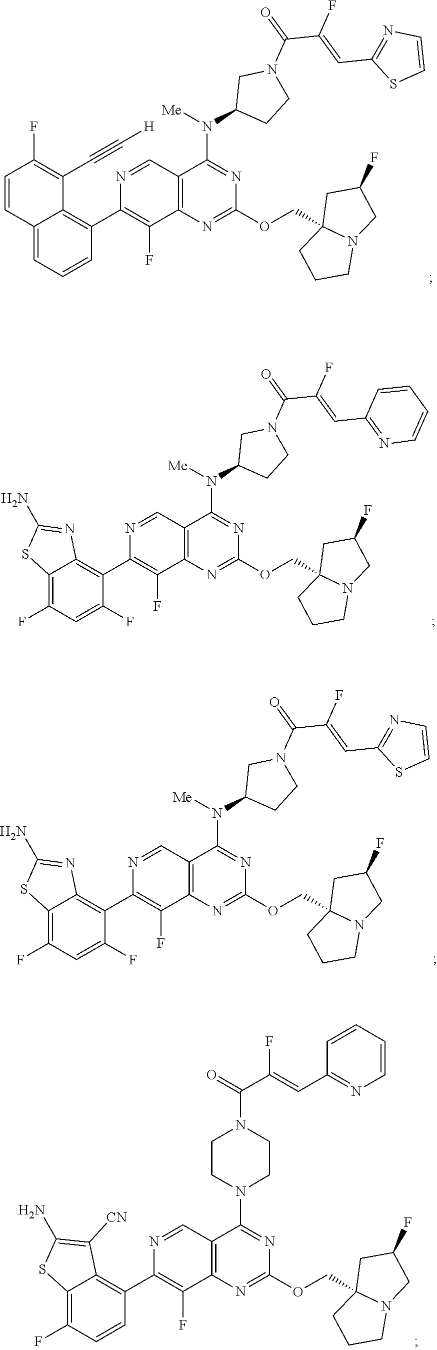 [SMILES: C#Cc1c(F)ccc2cccc(-c3ncc4c(N(C)[C@@H]5CCN(C(=O)/C(F)=C/c6nccs6)C5)nc(OC[C@@]56CCCN5C[C@H](F)C6)nc4c3F)c12 ; CN(c1nc(OC[C@@]23CCCN2C[C@H](F)C3)nc2c(F)c(-c3c(F)cc(F)c4sc(N)nc34)ncc12)[C@@H]1CCN(C(=O)/C(F)=C/c2ccccn2)C1 ; CN(c1nc(OC[C@@]23CCCN2C[C@H](F)C3)nc2c(F)c(-c3c(F)cc(F)c4sc(N)nc34)ncc12)[C@@H]1CCN(C(=O)/C(F)=C/c2nccs2)C1 ; N#Cc1c(N)sc2c(F)ccc(-c3ncc4c(N5CCN(C(=O)/C(F)=C/c6ccccn6)CC5)nc(OC[C@@]56CCCN5C[C@H](F)C6)nc4c3F)c12] 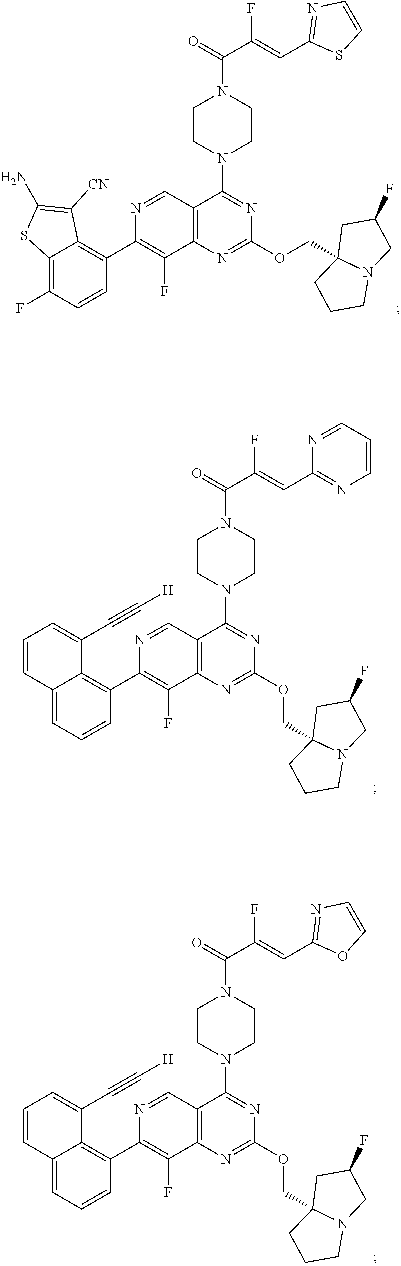 [SMILES: N#Cc1c(N)sc2c(F)ccc(-c3ncc4c(N5CCN(C(=O)/C(F)=C/c6nccs6)CC5)nc(OC[C@@]56CCCN5C[C@H](F)C6)nc4c3F)c12 ; C#Cc1cccc2cccc(-c3ncc4c(N5CCN(C(=O)/C(F)=C/c6ncccn6)CC5)nc(OC[C@@]56CCCN5C[C@H](F)C6)nc4c3F)c12 ; C#Cc1cccc2cccc(-c3ncc4c(N5CCN(C(=O)/C(F)=C/c6ncco6)CC5)nc(OC[C@@]56CCCN5C[C@H](F)C6)nc4c3F)c12] 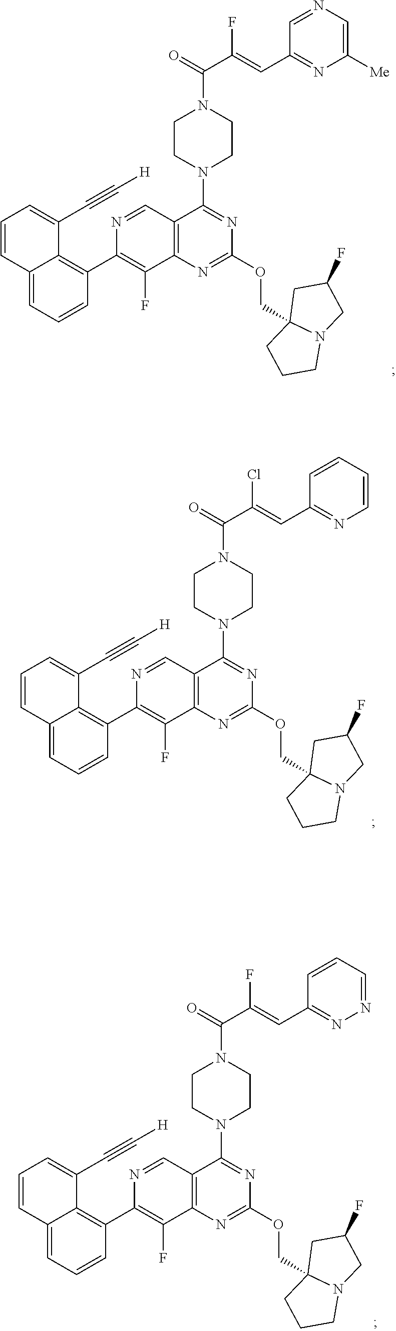 [SMILES: C#Cc1cccc2cccc(-c3ncc4c(N5CCN(C(=O)/C(F)=C/c6cncc(C)n6)CC5)nc(OC[C@@]56CCCN5C[C@H](F)C6)nc4c3F)c12 ; C#Cc1cccc2cccc(-c3ncc4c(N5CCN(C(=O)/C(Cl)=C/c6ccccn6)CC5)nc(OC[C@@]56CCCN5C[C@H](F)C6)nc4c3F)c12 ; C#Cc1cccc2cccc(-c3ncc4c(N5CCN(C(=O)/C(F)=C/c6cccnn6)CC5)nc(OC[C@@]56CCCN5C[C@H](F)C6)nc4c3F)c12] 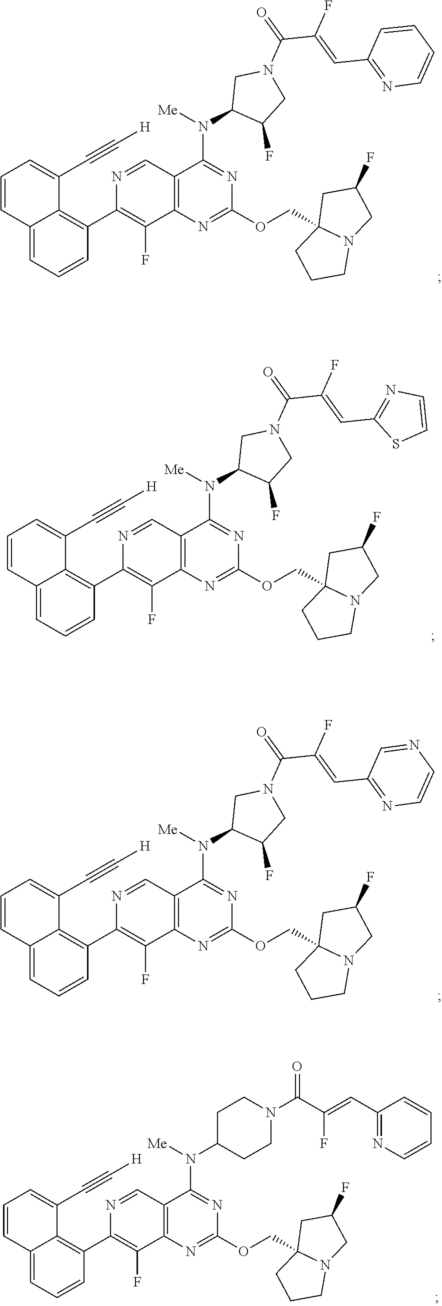 [SMILES: C#Cc1cccc2cccc(-c3ncc4c(N(C)[C@H]5CN(C(=O)/C(F)=C/c6ccccn6)C[C@H]5F)nc(OC[C@@]56CCCN5C[C@H](F)C6)nc4c3F)c12 ; C#Cc1cccc2cccc(-c3ncc4c(N(C)[C@H]5CN(C(=O)/C(F)=C/c6nccs6)C[C@H]5F)nc(OC[C@@]56CCCN5C[C@H](F)C6)nc4c3F)c12 ; C#Cc1cccc2cccc(-c3ncc4c(N(C)[C@H]5CN(C(=O)/C(F)=C/c6cnccn6)C[C@H]5F)nc(OC[C@@]56CCCN5C[C@H](F)C6)nc4c3F)c12 ; C#Cc1cccc2cccc(-c3ncc4c(N(C)C5CCN(C(=O)/C(F)=C/c6ccccn6)CC5)nc(OC[C@@]56CCCN5C[C@H](F)C6)nc4c3F)c12] 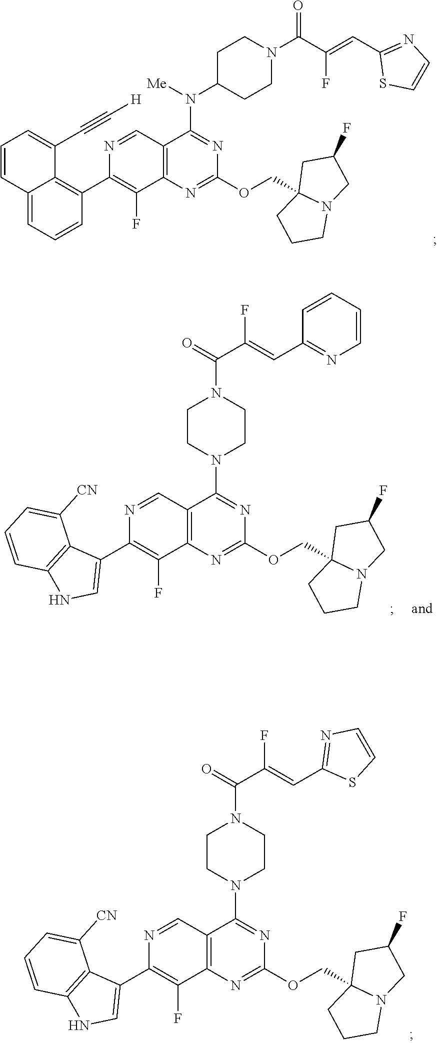 [SMILES: C#Cc1cccc2cccc(-c3ncc4c(N(C)C5CCN(C(=O)/C(F)=C/c6nccs6)CC5)nc(OC[C@@]56CCCN5C[C@H](F)C6)nc4c3F)c12 ; N#Cc1cccc2[nH]cc(-c3ncc4c(N5CCN(C(=O)/C(F)=C/c6ccccn6)CC5)nc(OC[C@@]56CCCN5C[C@H](F)C6)nc4c3F)c12 ; N#Cc1cccc2[nH]cc(-c3ncc4c(N5CCN(C(=O)/C(F)=C/c6nccs6)CC5)nc(OC[C@@]56CCCN5C[C@H](F)C6)nc4c3F)c12]
or a pharmaceutically acceptable salt thereof.

33. (canceled)

34. A pharmaceutical composition comprising a compound of claim 1, or a pharmaceutically acceptable salt thereof, and one or more pharmaceutically acceptable excipients.

35. (canceled)

36. A method of treating cancer expressing KRAS G12C mutation in a subject in need thereof, the method comprising administering to the subject a compound of claim 1, or a pharmaceutically acceptable salt thereof.

37. (canceled)

38. A method for treating a cancer susceptible to KRAS G12C inhibition in a subject in need thereof, the method comprising administering to the subject a compound of claim 1, or a pharmaceutically acceptable salt thereof.

39. A method for treating a cancer in a subject in need thereof, the method comprising administering to the subject a a-compound of claim 1, or a pharmaceutically acceptable salt thereof, wherein the cancer is lung cancer, colorectal cancer, pancreatic cancer, breast cancer, bladder cancer, cervical cancer, ovarian cancer, gastric cancer or cancer of the uterus.

40. (canceled)
